(19) United States
(12) Patent Application Publication
(10) Pub. No.: US 2018/0130968 A1
(43) Pub. Date: May 10, 2018

(54) ORGANIC ELECTROLUMINESCENCE ELEMENT AND ELECTRONIC DEVICE

(71) Applicant: IDEMITSU KOSAN CO., LTD., Tokyo (JP)

(72) Inventor: Takeshi IKEDA, Sodegaura-shi (JP)

(73) Assignee: IDEMITSU KOSAN CO., LTD., Tokyo (JP)

(21) Appl. No.: 15/866,413

(22) Filed: Jan. 9, 2018

Related U.S. Application Data
(63) Continuation of application No. PCT/JP2016/070348, filed on Jul. 8, 2016.

(30) Foreign Application Priority Data
Jul. 10, 2015 (JP) 2015-139245

(51) Int. Cl. H01L 51/50; H01L 51/52; C07C 15/28; C07C 211/54; C07C 255/37

(52) U.S. Cl. CPC H01L 51/5056; H01L 51/5072; H01L 51/5012; C07C 255/37; C07C 15/28; C07C 211/54; H01L 51/5203

(57) ABSTRACT

An organic electroluminescence device includes: an anode; a cathode; an emitting layer disposed between the anode and the cathode; and a hole transporting layer disposed between the anode and the emitting layer. The emitting layer contains a first compound represented by a formula (1) and a fluorescent second compound. The hole transporting layer contains a third compound represented by a formula (3). In the formula (1), Z₁ is represented by a formula (1a). A cyclic structure represented by a formula (1b) or (1c) may be fused to Z₁. Each of X₁ and X₂ is an oxygen atom, a sulfur atom, NR_{A} or CR_{B}R_{C}.

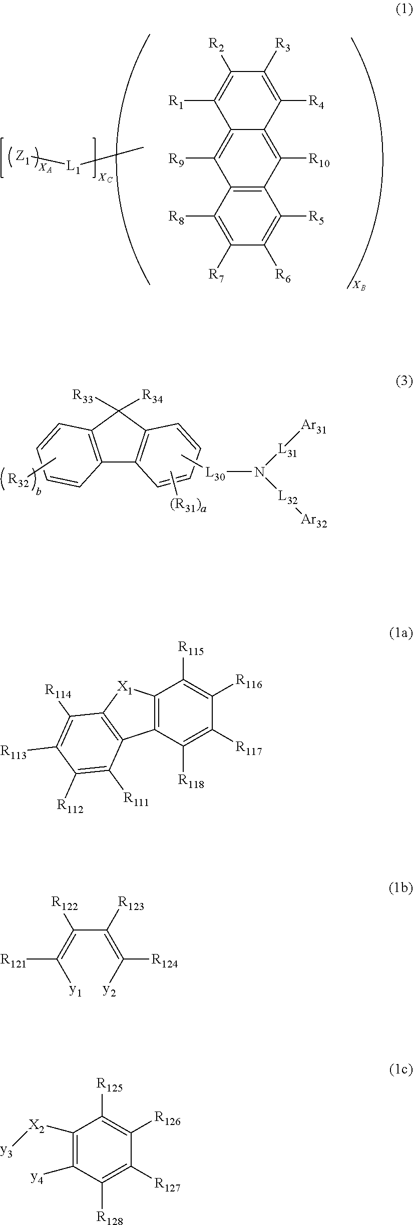

### CROSS-REFERENCE TO RELATED APPLICATIONS

The present application is a continuation application filed under 35 U.S.C. § 111(a) claiming the benefit under 35 U.S.C. §§ 120 and 365(c) of International Patent Application No. PCT/JP2016/070348, filed Jul. 8, 2016, which is based upon and claims the benefit of priority to Japanese Patent Application No. 2015-139245, filed Jul. 10, 2015, the entireties of which are hereby incorporated by reference.

### TECHNICAL FIELD

The present invention relates to an organic electroluminescence device and an electronic device.

### BACKGROUND ART

An organic electroluminescence device (hereinafter, occasionally abbreviated as organic EL device) using an organic substance is highly expected to be used as an inexpensive solid-emitting full-color display device having a large area and has been variously developed. A typical organic EL device includes an emitting layer and a pair of opposing electrodes (i.e., an anode and a cathode) between which the emitting layer is interposed. When an electric field is applied on both electrodes, electrons are injected from the cathode while holes are injected from the anode. Recombination of the electrons and the holes in the emitting layer generates an excited state. Energy generated when the excited state is returned to the ground state is radiated as light.

A typical organic EL device requires a higher drive voltage but exhibits lower luminescence intensity and luminous efficiency than those of an inorganic light-emitting diode. In recent organic EL devices, an improvement in a compound used for forming an organic layer has been made. For instance, in Patent Literatures 1 to 3, an anthracene derivative and a luminescent material contained in an emitting layer are studied to improve the performance of an organic EL device.

### CITATION LIST

### Patent Literature(S)

Patent Literature 1: International Publication No. WO2014/034893

Patent Literature 2: International Publication No. WO2014/141725

Patent Literature 3: International Publication No. WO2010/137285

### SUMMARY OF THE INVENTION

### Problems to be Solved by the Invention

An object of the invention is to provide an organic electroluminescence device that exhibits a high luminous efficiency with a low drive voltage, and an electronic device using the organic electroluminescence device.

### Means for Solving the Problems

An organic electroluminescence device includes: an anode; a cathode; an emitting layer disposed between the anode and the cathode; and a hole transporting layer disposed between the anode and the emitting layer, in which the emitting layer contains a first compound represented by a formula (1) below and a fluorescent second compound, and the hole transporting layer contains a third compound represented by a formula (3) below.

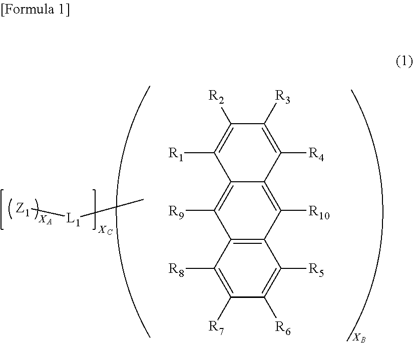

In the formula (1): one of R₁ to R₁₀ is a single bond to L₁, and the rest of R₁ to R₁₀ not bonded to L₁ are each independently a hydrogen atom or a substituent; ones of R₁ to R₁₀ each in a form of the substituent are each independently selected from the group consisting of a halogen atom, a hydroxyl group, a cyano group, a substituted or unsubstituted amino group, a substituted or unsubstituted alkyl group having 1 to 30 carbon atoms, a substituted or unsubstituted alkoxy group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted aromatic hydrocarbon group having 6 to 30 ring carbon atoms, and a substituted or unsubstituted heterocyclic group having 5 to 30 ring atoms; L₁ is a single bond or a linking group; L₁ in a form of the linking group is a substituted or unsubstituted aromatic hydrocarbon group having 6 to 30 ring carbon atoms, or a substituted or unsubstituted heterocyclic group having 5 to 30 ring atoms; Z₁ is represented by a formula (1a) below; x_{A}, x_{B} and x_{C} are each independently an integer of 1 to 4; plural Z₁ are optionally the same or different; plural structures represented by [(Z₁)x_{A}-L₁-] are optionally the same or different; and plural cyclic structures in parentheses with a subscript x_{B} are optionally mutually the same or different.

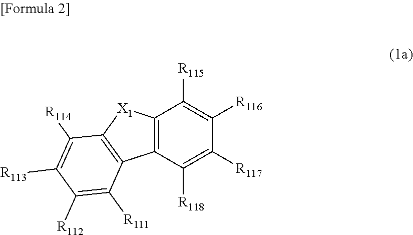

In the formula (1a): X₁ is an oxygen atom, a sulfur atom, NR_{A} or CR_{B}R_{C}; R₁₁₁ to R₁₁₈, R_{A}, R_{B} and R_{C} are each independently a hydrogen atom, a substituent or a single bond to L₁; R₁₁₁ to R₁₁₈, R_{A}, R_{B} and R_{C} each in a form of the substituent are each independently selected from the group consisting of the substituents listed for R₁ to R₁₀ each in a form of the substituent; and when at least one of a pair of R₁₁₁ and R₁₁₂, a pair of R₁₁₂ and R₁₁₃, a pair of R₁₁₃ and R₁₁₄, a pair of R₁₁₅ and R₁₁₆, a pair of R₁₁₆ and R₁₁₇ or a pair of R₁₁₇ and R₁₁₈ are both substituents, the substituents are optionally bonded to each other to form a ring represented by a formula (1b) or (1c) below.

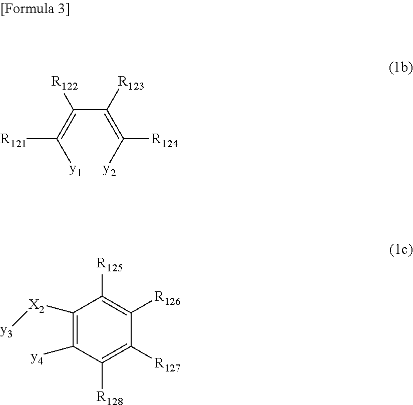

In the formula (1b): y₁ and y₂ in the formula (1b) each represent a position where a cyclic structure of Z₁ represented by the formula (1a) is bonded; y₃ and y₄ in the formula (1c) each represent a position where the cyclic structure of Z₁ represented by the formula (1a) is bonded; X₂ in the formula (1c) is an oxygen atom, a sulfur atom, NR_{D} or CR_{E}R_{F}; R₁₂₁ to R₁₂₄, R₁₂₅ to R₁₂₈, R_{D}, R_{E} and R_{F} in the formulae (1b) and (1c) are each independently a hydrogen atom, a substituent or a single bond to L₁; R₁₂₁ to R₁₂₄, R₁₂₅ to R₁₂₈, R_{D}, R_{E} and R_{F} each in a form of the substituent are each independently selected from the group consisting of the substituents listed for R₁ to R₁₀ each in a form of the substituent; and when a ring represented by the formula (1b) is formed, one of R₁₁₁ to R₁₁₈ and R₁₂₁ and R₁₂₄ not forming the ring is a single bond to L₁, and when a ring represented by the formula (1c) is formed, one of R₁₁₁ to R₁₁₈ and R₁₂₅ and R₁₂₈ not forming the ring is a single bond to L₁.

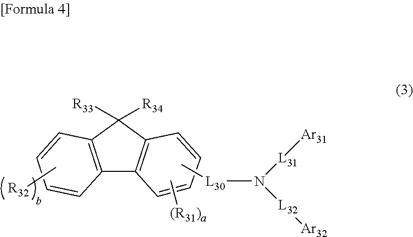

In the formula (3): R₃₁ to R₃₂ are each independently a hydrogen atom or a substituent; R₃₁ to R₃₂ each in a form of the substituent are each independently selected from the group consisting of the substituents listed for R₁ to R₈ each in a form of the substituent; a is 3; plural R₃₁ are optionally mutually the same or different; R₃₁ each in a form of the substituent are optionally bonded together to form a cyclic structure; b is 4; plural R₃₂ are optionally mutually the same or different; R₃₂ each in a form of the substituent are optionally bonded together to form a cyclic structure; R₃₃ to R₃₄ are each independently a hydrogen atom or a substituent; R₃₃ to R₃₄ each in a form of the substituent are each independently selected from the group consisting of a substituted or unsubstituted alkyl group having 1 to 20 carbon atoms, a substituted or unsubstituted fluoroalkyl group having 1 to 20 carbon atoms, a substituted or unsubstituted aromatic hydrocarbon group having 6 to 50 ring carbon atoms, a substituted or unsubstituted heterocyclic group having 3 to 50 ring atoms, and a cyano group; R₃₃ and R₃₄ each in a form of the substituent are optionally bonded together to form a cyclic structure; L₃₀, L₃₁ and L₃₂ are each independently a single bond or a linking group; L₃₀, L₃₁ and L₃₂ each in a form of the linking group are each independently a substituted or unsubstituted aromatic hydrocarbon group having 6 to 30 ring carbon atoms or a substituted or unsubstituted heterocyclic group having 5 to 30 ring atoms; and Ar₃₁ and Ar₃₂ are each independently a substituted or unsubstituted aromatic hydrocarbon group having 6 to 30 ring carbon atoms or a substituted or unsubstituted heterocyclic group having 5 to 30 ring atoms.

According to another aspect of the invention, an electronic device includes the above organic electroluminescence device.

The above aspects of the invention can provide an organic electroluminescence device that exhibits a high luminous efficiency with a low drive voltage, and an electronic device using the organic electroluminescence device.

### BRIEF DESCRIPTION OF DRAWING

FIG. 1 schematically shows an exemplary arrangement of an organic EL device according to a first exemplary embodiment of the invention.

FIG. 2 schematically shows an exemplary arrangement of an organic EL device according to a second exemplary embodiment of the invention.

### DESCRIPTION OF EMBODIMENT(S)

### First Exemplary Embodiment

### Organic EL Device

An organic EL device includes an anode, a cathode, and an organic layer interposed between the anode and the cathode. The organic layer includes at least one layer formed of an organic compound. The organic layer may further include an inorganic compound. The organic layer of the organic EL device of the first exemplary embodiment includes at least one emitting layer and at least one hole transporting layer. The organic layer may, for instance, consist of the emitting layer and the hole transporting layer or may further include one of layers usable in typical organic EL devices, such as a hole injecting layer, an electron injecting layer, an electron transporting layer, a hole blocking layer, and an electron blocking layer. Each of the above layers in the organic EL device may be formed of a single layer or a plurality of layers. For instance, the organic EL device may include two hole transporting layers such as a first hole transporting layer and a second hole transporting layer.

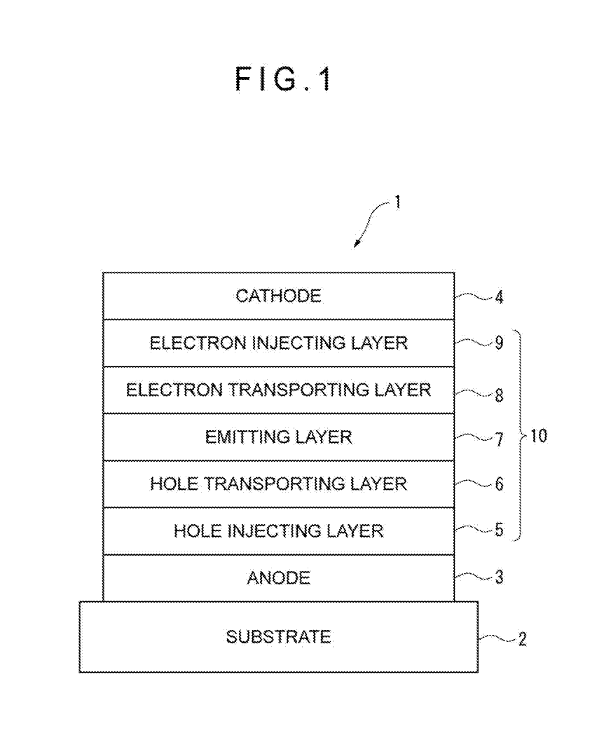

FIG. 1 schematically shows an arrangement of an exemplary organic EL device according to the first exemplary embodiment.

An organic EL device 1 includes a light-transmissive substrate 2, an anode 3, a cathode 4 and an organic layer 10 disposed between the anode 3 and the cathode 4. The organic layer 10 includes a hole injecting layer 5, a hole transporting layer 6, an emitting layer 7, an electron transporting layer 8 and an electron injecting layer 9, which are laminated in this sequence from the anode 3.

### Emitting Layer

The emitting layer 7 of the organic EL device 1 contains a first compound represented by a formula (1) below and a fluorescent second compound.

### First Compound

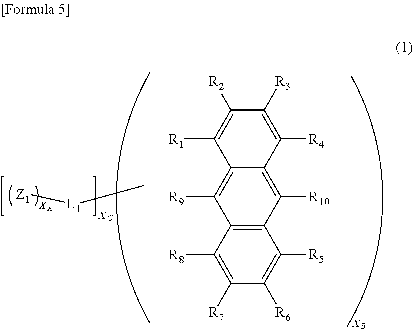

In the formula (1), one of R₁ to R₁₀ is a single bond to L₁, and the rest of R₁ to R₁₀ not bonded to L₁ are each independently a hydrogen atom or a substituent. R₁ to R₁₀ in a form of substituents are each independently selected from the group consisting of a halogen atom, hydroxyl group, cyano group, substituted or unsubstituted amino group, substituted or unsubstituted alkyl group having 1 to 30 carbon atoms, substituted or unsubstituted alkoxy group having 1 to 30 carbon atoms, substituted or unsubstituted aryloxy group having 6 to 30 ring carbon atoms, substituted or unsubstituted arylthio group having 6 to 30 ring carbon atoms, substituted or unsubstituted aromatic hydrocarbon group having 6 to 30 ring carbon atoms, and substituted or unsubstituted heterocyclic group having 5 to 30 ring atoms.

L₁ is a single bond or a linking group. L₁ in a form of the linking group is a substituted or unsubstituted aromatic hydrocarbon group having 6 to 30 ring carbon atoms or a substituted or unsubstituted heterocyclic group having 5 to 30 ring atoms.

Z₁ is represented by a formula (1a) below, in which x_{A}, x_{B} and x_{C} are each independently an integer of 1 to 4, plural Z₁ may be mutually the same or different, plural structures represented by [(Z₁)x_{A}-L₁-] may be mutually the same or different, and plural cyclic structures in parentheses with the subscript x_{B} may be mutually the same or different.

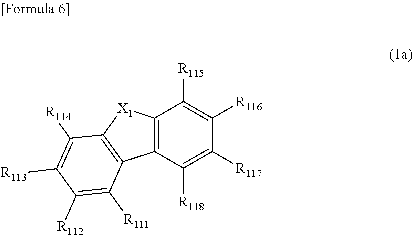

In the formula (1a): X₁ is an oxygen atom, a sulfur atom, NR_{A} or CR_{B}R_{C}; R₁₁₁ to R₁₁₈, R_{A}, R_{B} and R_{C} are each independently a hydrogen atom, a substituent or a single bond to L₁; R₁₁₁ to R₁₁₈, R_{A}, R_{B} and R_{C} in a form of the substituents are each independently selected from the group consisting of the substituents listed for R₁ to R₁₀ in a form of the substituents; and when at least one of a pair of R₁₁₁ and R₁₁₂, a pair of R₁₁₂ and R₁₁₃, a pair of R₁₁₃ and R₁₁₄, a pair of R₁₁₅ and R₁₁₆, a pair of R₁₁₆ and R₁₁₇ or a pair of R₁₁₇ and R₁₁₈ are both substituents, the substituents may be bonded to each other to form a ring represented by a formula (1b) or (1c) below.

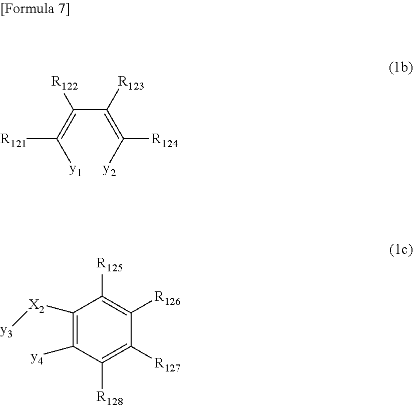

y₁ and y₂ in the formula (1b) each represent a position where the cyclic structure of Z₁ represented by the formula (1a) is bonded; y₃ and y₄ in the formula (1c) each represent a position where the cyclic structure of Z₁ represented by the formula (1a) is bonded; X₂ in the formula (1c) is an oxygen atom, a sulfur atom, NR_{D} or CR_{E}R_{F}; R₁₂₁ to R₁₂₄, R₁₂₅ to R₁₂₈, R_{D}, R_{E} and R_{F} in the formulae (1b) and (1c) are each independently a hydrogen atom, a substituent or a single bond to L₁; R₁₂₁ to R₁₂₄, R₁₂₅ to R₁₂₈, R_{D}, R_{E} and R_{F} in a form of the substituents are each independently selected from the group consisting of the substituents listed for R₁ to R₁₀ in a form of the substituents; and when a ring represented by the formula (1b) is formed, one of R₁₁₁ to R₁₁₈ and R₁₂₁ and R₁₂₄ not forming the ring is a single bond to L₁, and when a ring represented by the formula (1c) is formed, one of R₁₁₁ to R₁₁₈ and R₁₂₅ to R₁₂₈ not forming the ring is a single bond to L₁.

In the formula (1a): at least one of a pair of R₁₁₁ and R₁₁₂, a pair of R₁₁₂ and R₁₁₃, a pair of R₁₁₃ and R₁₁₄, a pair of R₁₁₅ and R₁₁₆, a pair of R₁₁₆ and R₁₁₇ or a pair of R₁₁₇ and R₁₁₈ are both substituents; and at least one of the pair of R₁₁₁ and R₁₁₂ both being the substituents, the pair of R₁₁₂ and R₁₁₃ both being the substituents, the pair of R₁₁₃ and R₁₁₄ both being the substituents, the pair of R₁₁₅ and R₁₁₆ both being the substituents, the pair of R₁₁₆ and R₁₁₇ both being the substituents or the pair of R₁₁₇ and R₁₁₈ both being the substituents are preferably bonded to each other to form a ring represented by the formula (1b) or (1c).

Z₁ in the first compound is preferably one selected from the group consisting of groups represented by formulae (11) to (13) below.

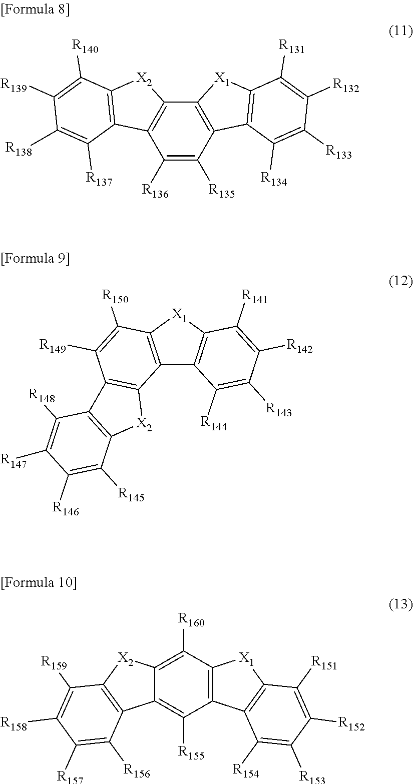

In the formulae (11) to (13): R₁₃₁ to R₁₄₀, R₁₄₁ to R₁₅₀ and R₁₅₁ to R₁₆₀ represent the same as the rest of R₁ to R₁₀ not bonded to L₁ in the formula (1); at least one of R₁₃₁ to R₁₄₀, at least one of R₁₄₁ to R₁₅₀ or at least one of R₁₅₁ to R₁₆₀ is a single bond to L₁; X₁ represents the same as X₁ in the formula (1a); X₂ represents the same as X₂ in the formula (1c); and X₁ and X₂ are mutually the same or different.

When a ring represented by the formula (1c) is formed in the first compound, X₁ and X₂ are each preferably an oxygen atom.

Z₁ in the first compound is preferably one selected from the group consisting of groups represented by formulae (14) to (16) below.

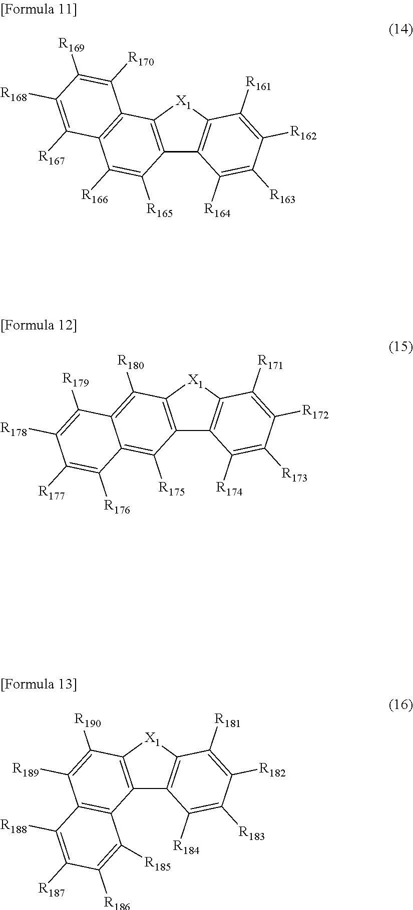

In the formulae (14) to (16), R₁₆₁ to R₁₇₀, R₁₇₁ to R₁₈₀ and R₁₈₁ to R₁₉₀ each independently represent the same as R₁ to R₁₀ not bonded to L₁ in the formula (1). It should be noted that one of R₁₆₁ to R₁₇₀, one of R₁₇₁ to R₁₈₀ and one of R₁₈₁ to R₁₉₀ are each a single bond to L₁, and X₁ in the formulae (14) to (16) represents the same as X₁ in the formula (1a).

x_{B} in the first compound is preferably 1.

x_{A} in the first compound is preferably 1 or 2.

In the first compound, L₁ is preferably bonded to one of R₁ to R₄, more preferably bonded to R₂.

In the first compound, L₁ is preferably bonded to R₉.

The first compound is preferably represented by a formula (10) below.

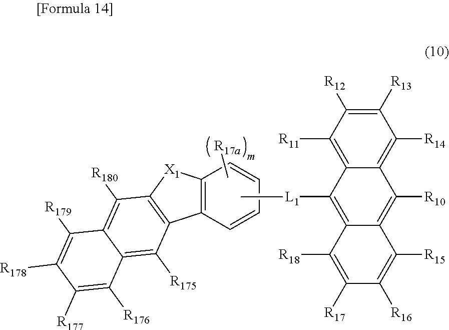

In the formula (10): R₁₁ to R₁₈ are each independently a hydrogen atom or a substituent; R₁₁ to R₁₈ in a form of the substituents are each independently selected from the group consisting of a halogen atom, hydroxyl group, cyano group, substituted or unsubstituted amino group, substituted or unsubstituted alkyl group having 1 to 30 carbon atoms, substituted or unsubstituted alkoxy group having 1 to 30 carbon atoms, substituted or unsubstituted aryloxy group having 6 to 30 ring carbon atoms, substituted or unsubstituted arylthio group having 6 to 30 ring carbon atoms, substituted or unsubstituted aromatic hydrocarbon group having 6 to 30 ring carbon atoms, and substituted or unsubstituted heterocyclic group having 5 to 30 ring atoms; L₁ is a single bond or a linking group; L₁ in a form of the linking group is a substituted or unsubstituted aromatic hydrocarbon group having 6 to 30 ring carbon atoms or a substituted or unsubstituted heterocyclic group having 5 to 30 ring atoms; R₁₀ is a substituted or unsubstituted aromatic hydrocarbon group having 6 to 30 ring carbon atoms or a substituted or unsubstituted heterocyclic group having 5 to 30 ring atoms; R₁₇ₐ is a hydrogen atom, a substituent or a single bond to L₁; R₁₇ₐ in a form of the substituent is selected from the group consisting of the substituents listed for R₁₁ to R₁₈ in a form of the substituents; m is 3; plural R₁₇ₐ may be mutually the same or different; X₁ represents the same as X₁ in the formula (1a); R₁₇₅ to R₁₈₀ are each independently a hydrogen atom or a substituent; and R₁₇₅ to R₁₈₀ in a form of the substituents are each independently selected from the group consisting of the substituents listed for R₁₁ to R₁₈ in a form of the substituents.

The first compound is preferably represented by a formula (101) or a formula (102) below.

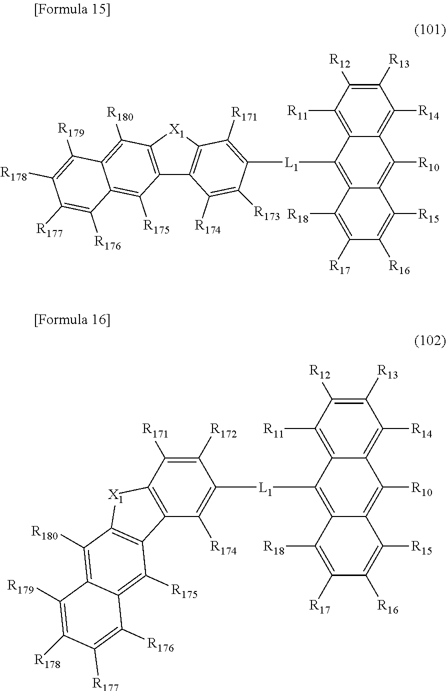

In the formulae (101) and (102), R₁₀, R₁₁ to R₁₈, L₁, X₁ and R₁₇₅ to R₁₈₀ respectively represent the same as R₁₀, R₁₁ to R₁₈, L₁, X₁ and R₁₇₅ to R₁₈₀ in the formula (10), R₁₇₁ to R₁₇₄ are each independently a hydrogen atom or a substituent, and R₁₇₁ to R₁₇₄ in a form of the substituents are selected from the group consisting of the substituents listed for R₁₁ to R₁₈ in a form of the substituents.

L₁ is also preferably a single bond.

The first compound is also preferably represented by a formula (103) or a formula (104) below.

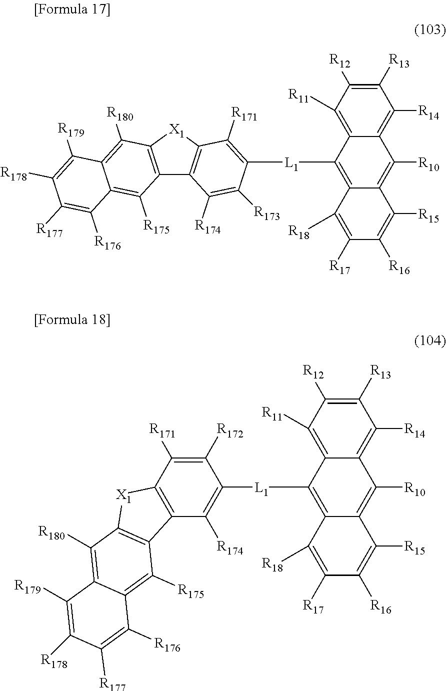

In the formulae (103) and (104), R₁₀, R₁₁ to R₁₈, X₁ and R₁₇₅ to R₁₈₀ respectively represent the same as R₁₀, R₁₁ to R₁₈, X₁ and R₁₇₅ to R₁₈₀ in the formula (10), R₁₇₁ to R₁₇₄ are each independently a hydrogen atom or a substituent, and R₁₇₁ to R₁₇₄ in a form of the substituents are selected from the group consisting of the substituents listed for R₁₁ to R₁₈ in a form of the substituents.

R₁₀ in the first compound is preferably one selected from the group consisting of a substituted or unsubstituted aromatic hydrocarbon group having 6 to 30 ring carbon atoms and a substituted or unsubstituted heterocyclic group having 5 to 30 ring atoms, more preferably a substituted or unsubstituted aromatic hydrocarbon group having 6 to 20 ring carbon atoms, further preferably a substituted or unsubstituted aromatic hydrocarbon group having 6 to 14 ring carbon atoms, still further preferably a substituted or unsubstituted aromatic hydrocarbon group having 6 to 12 ring carbon atoms.

R₁₀ in the first compound is preferably a group represented by a formula (1d) below.

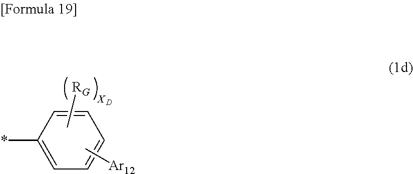

In the formula (1d), Ar₁₂ is one selected from the group consisting of a substituted or unsubstituted aromatic hydrocarbon group having 6 to 30 ring carbon atoms and a substituted or unsubstituted heterocyclic group having 5 to 30 ring atoms, R_{G} is a hydrogen atom or a substituent, R_{G} in a form of the substituent is selected from the group consisting of the substituents listed for R₁ to R₁₀ in a form of the substituents, x_{D} is 4, and plural R_{G} may be mutually the same or different.

R₁₀ in the first compound is also preferably a substituted or unsubstituted fused aromatic hydrocarbon group having 10 to 30 ring carbon atoms, and also preferably a substituent selected from the group consisting of a substituted or unsubstituted naphthyl group, substituted or unsubstituted phenanthryl group, substituted or unsubstituted benzanthryl group, and substituted or unsubstituted 9,9-dimethylfluorenyl group.

Regarding R₁₀, the substituent meant by “substituted or unsubstituted” is preferably one selected from the group consisting of an aromatic hydrocarbon group, alkyl group, halogen atom, alkylsilyl group, arylsilyl group and cyano group, more preferably selected from the group consisting of an aromatic hydrocarbon group and an alkyl group. R₁₀ is also preferably unsubstituted.

R₁₀ is also preferably one selected from the group consisting of groups represented by formulae (11a) to (11k), (11m), (11n) and (11p) below. In the formulae (11a) to (11k), (11m), (11n) and (11p), * represents a position for bonding to position 9 or 10 of the anthracene ring.

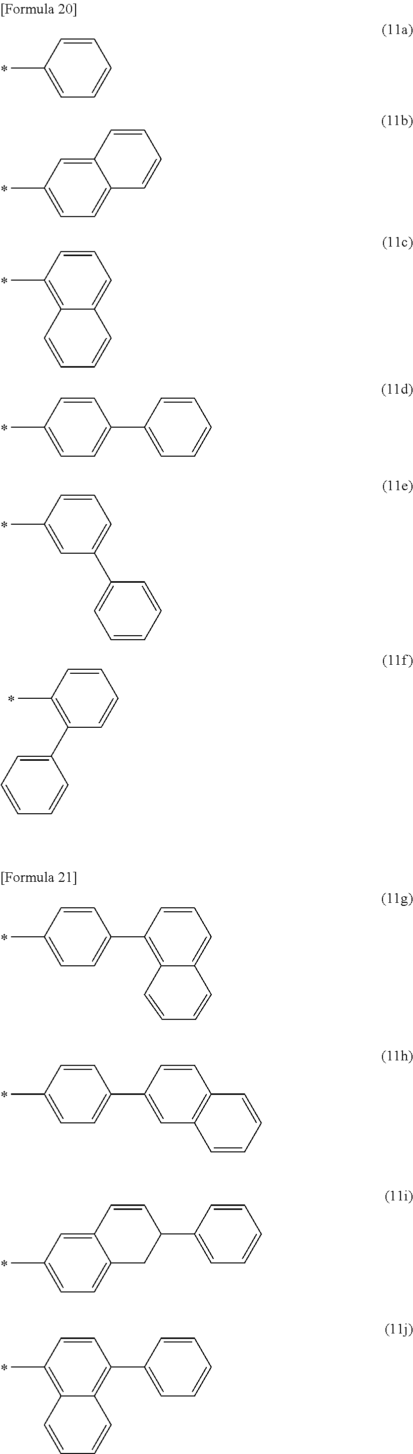

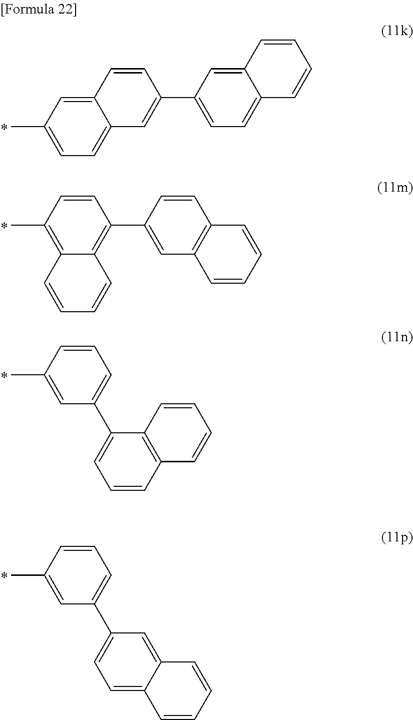

R₁₁ to R₁₈ are each preferably a hydrogen atom or an alkyl group having 1 to 30 carbon atoms, more preferably a hydrogen atom.

R₁₇₁ to R₁₈₀ are each preferably a hydrogen atom or an alkyl group having 1 to 30 carbon atoms unless being a single bond to L₁, and more preferably a hydrogen atom.

X₁ is preferably an oxygen atom or a sulfur atom, more preferably an oxygen atom.

The first compound with X₁ being an oxygen atom or a sulfur atom is believed to achieve molecular planarity across a larger area when the naphthobenzofuran or naphthobenzothiophene skeleton is bonded to the anthracene skeleton in the predetermined position (position 9 or 10), improving intermolecule packing and, consequently, improving electron-injection performance and electron-transport performance. An organic EL device using such a first compound is thus believed to exhibit an improved luminous efficiency with a reduced drive voltage.

Specific examples of the first compound are given below. It should be noted that the first compound of the invention is not limited to the examples.

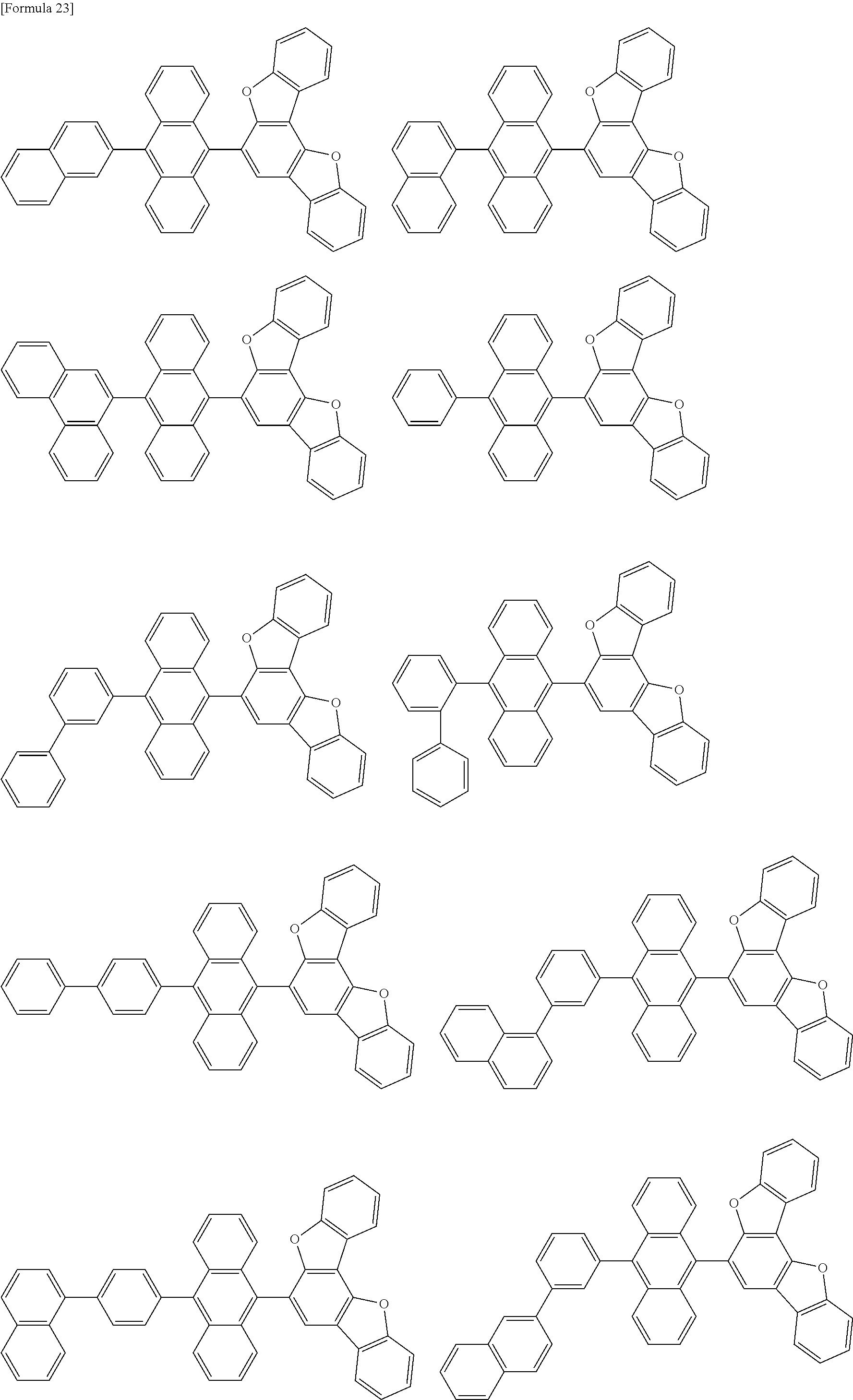

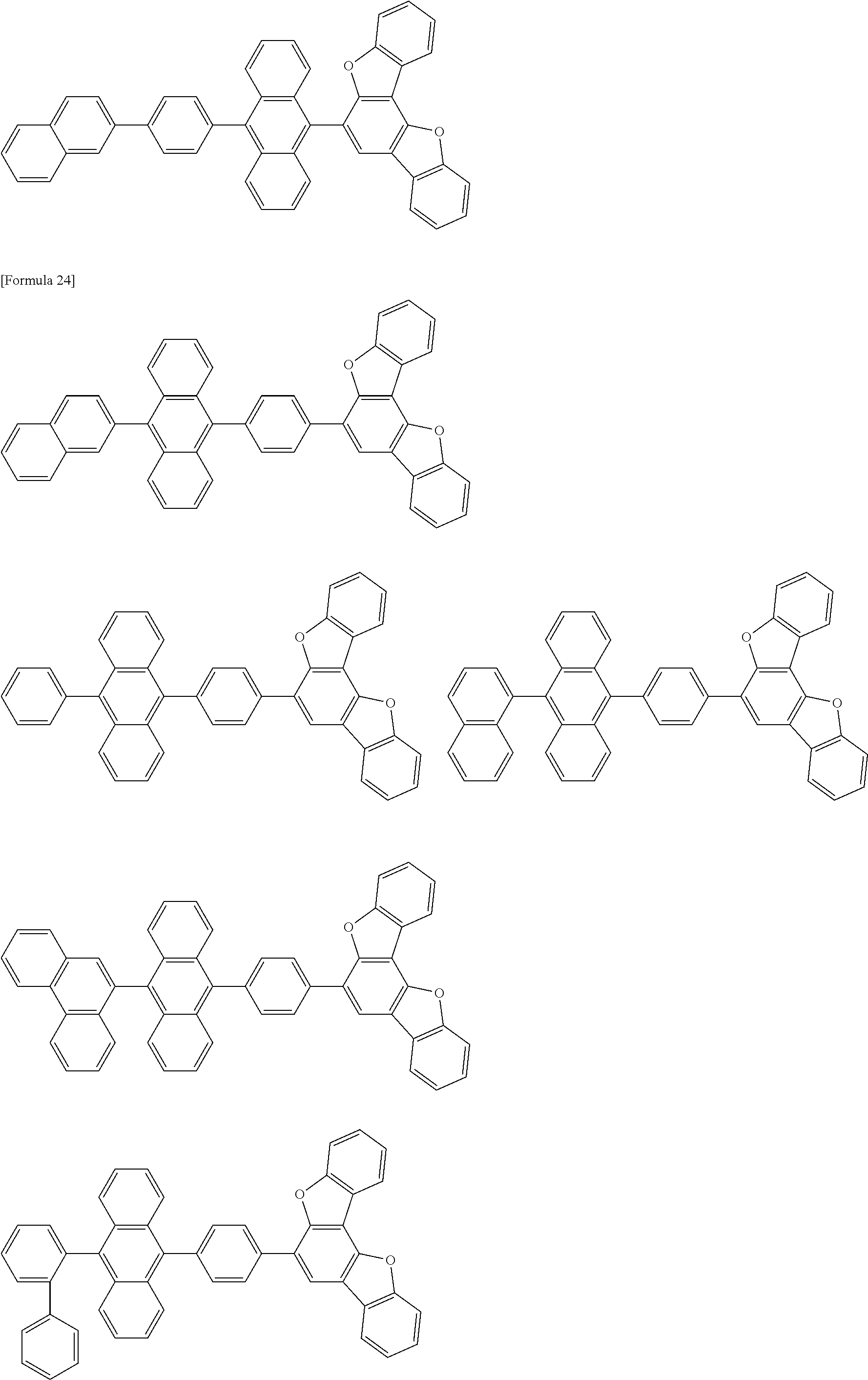

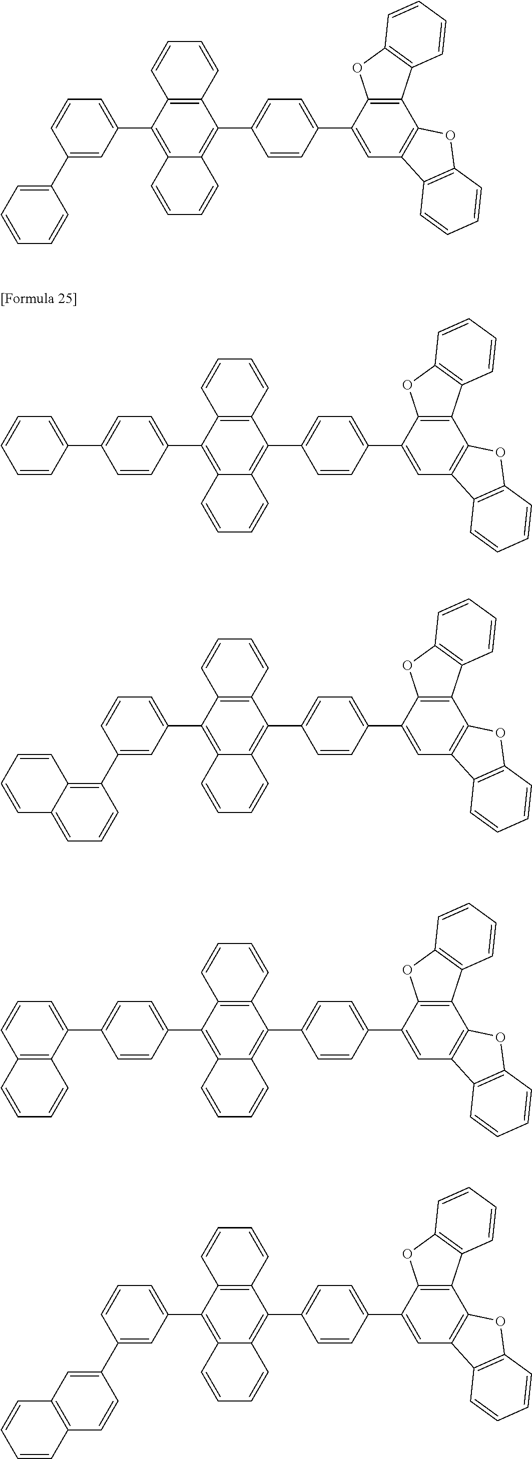 [SMILES: c1ccc(-c2cccc(-c3c4ccccc4c(-c4ccc(-c5cc6c7ccccc7oc6c6c5oc5ccccc56)cc4)c4ccccc34)c2)cc1 ; c1ccc(-c2ccc(-c3c4ccccc4c(-c4ccc(-c5cc6c7ccccc7oc6c6c5oc5ccccc56)cc4)c4ccccc34)cc2)cc1 ; c1cc(-c2cccc3ccccc23)cc(-c2c3ccccc3c(-c3ccc(-c4cc5c6ccccc6oc5c5c4oc4ccccc45)cc3)c3ccccc23)c1 ; c1ccc2c(-c3ccc(-c4c5ccccc5c(-c5ccc(-c6cc7c8ccccc8oc7c7c6oc6ccccc67)cc5)c5ccccc45)cc3)cccc2c1 ; c1cc(-c2ccc3ccccc3c2)cc(-c2c3ccccc3c(-c3ccc(-c4cc5c6ccccc6oc5c5c4oc4ccccc45)cc3)c3ccccc23)c1]

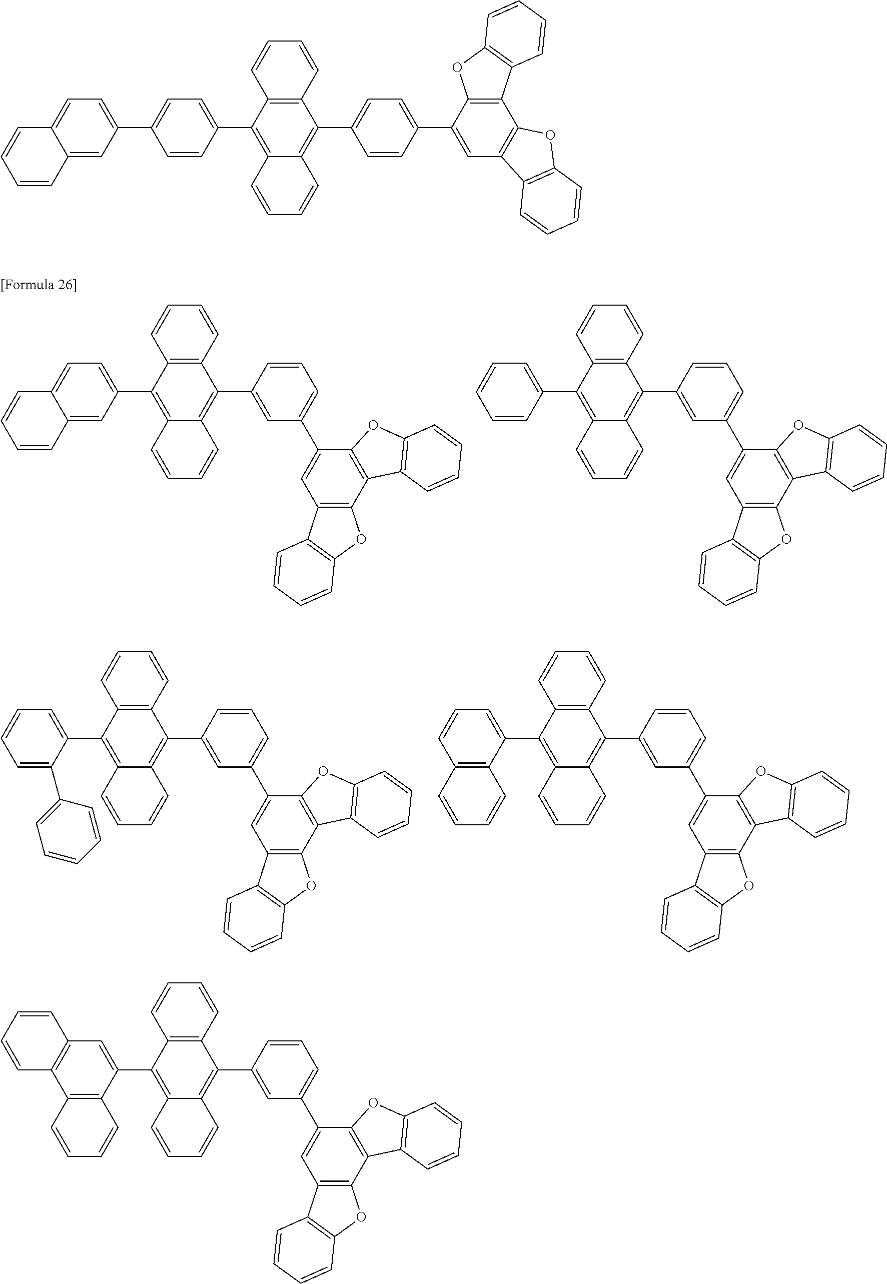

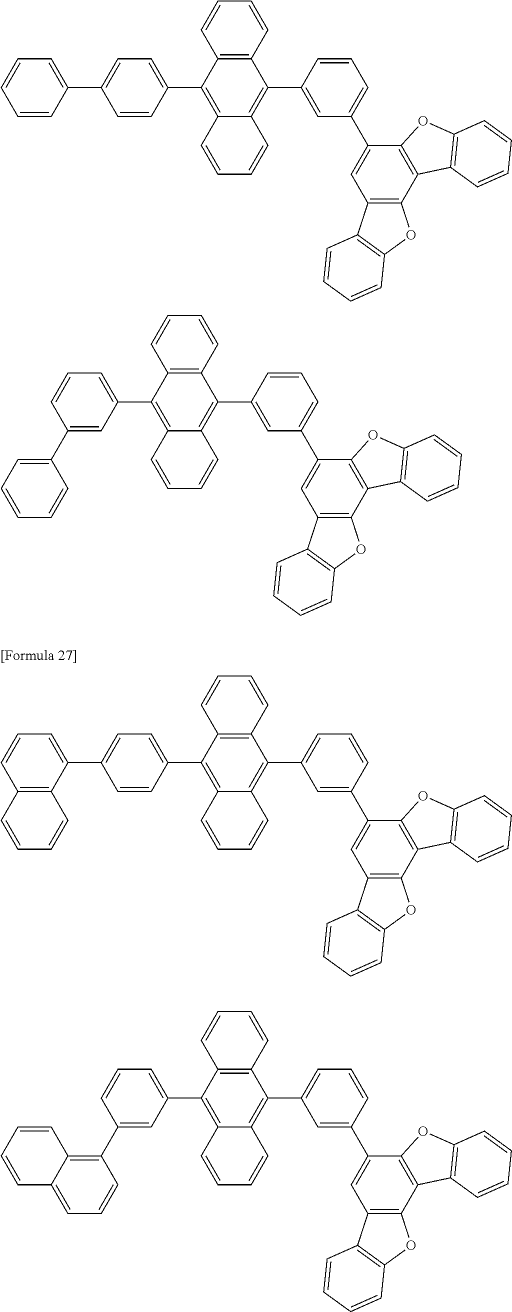 [SMILES: c1ccc(-c2ccc(-c3c4ccccc4c(-c4cccc(-c5cc6c7ccccc7oc6c6c5oc5ccccc56)c4)c4ccccc34)cc2)cc1 ; c1ccc(-c2cccc(-c3c4ccccc4c(-c4cccc(-c5cc6c7ccccc7oc6c6c5oc5ccccc56)c4)c4ccccc34)c2)cc1 ; c1cc(-c2c3ccccc3c(-c3ccc(-c4cccc5ccccc45)cc3)c3ccccc23)cc(-c2cc3c4ccccc4oc3c3c2oc2ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

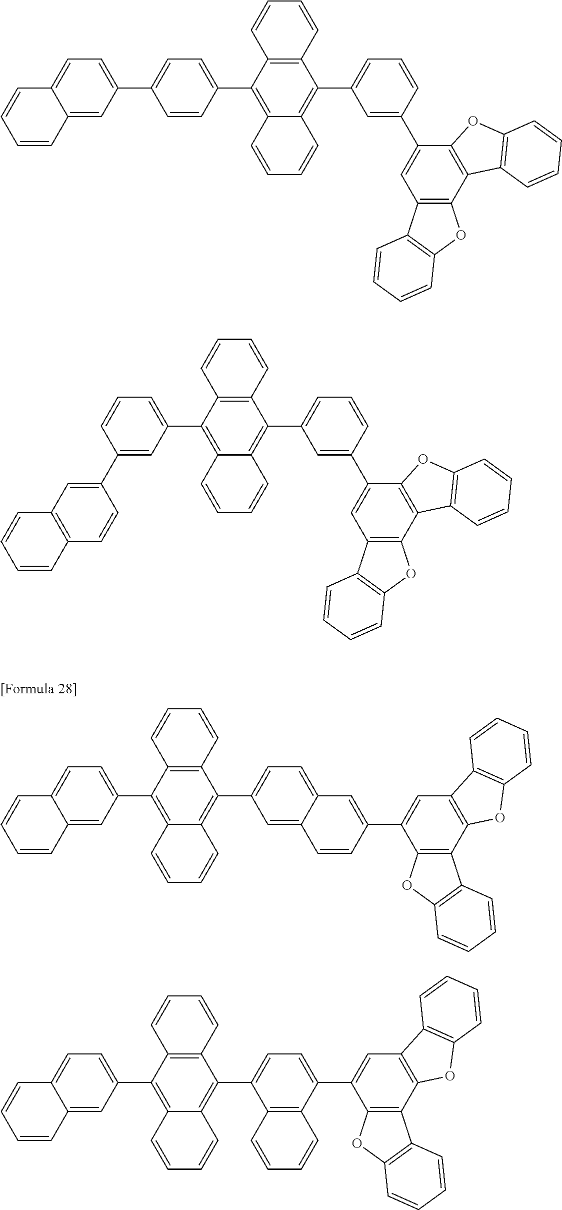

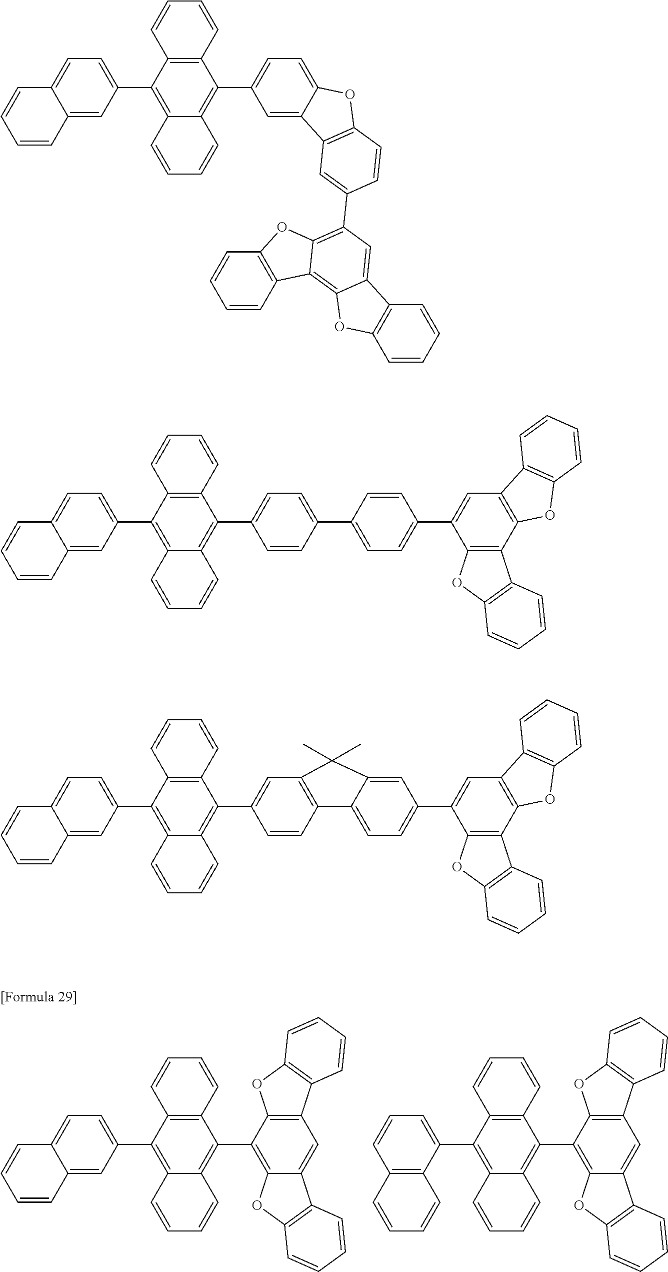

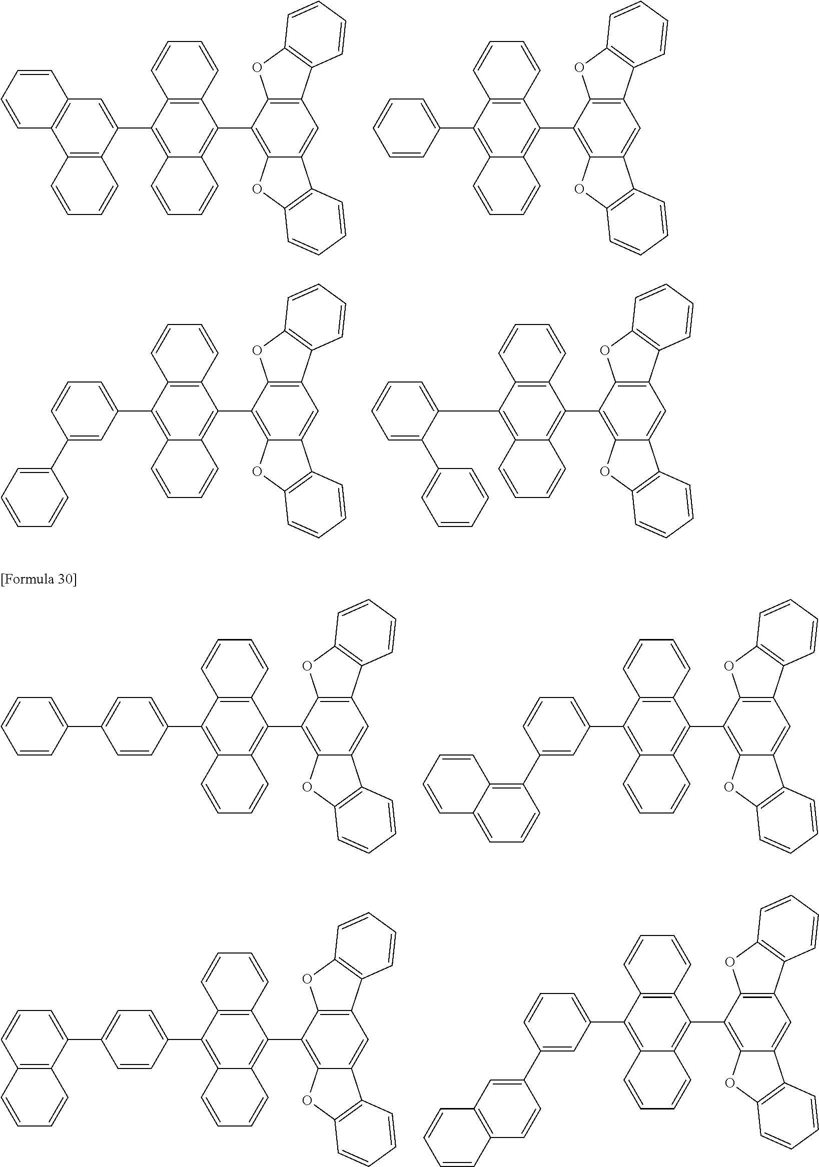

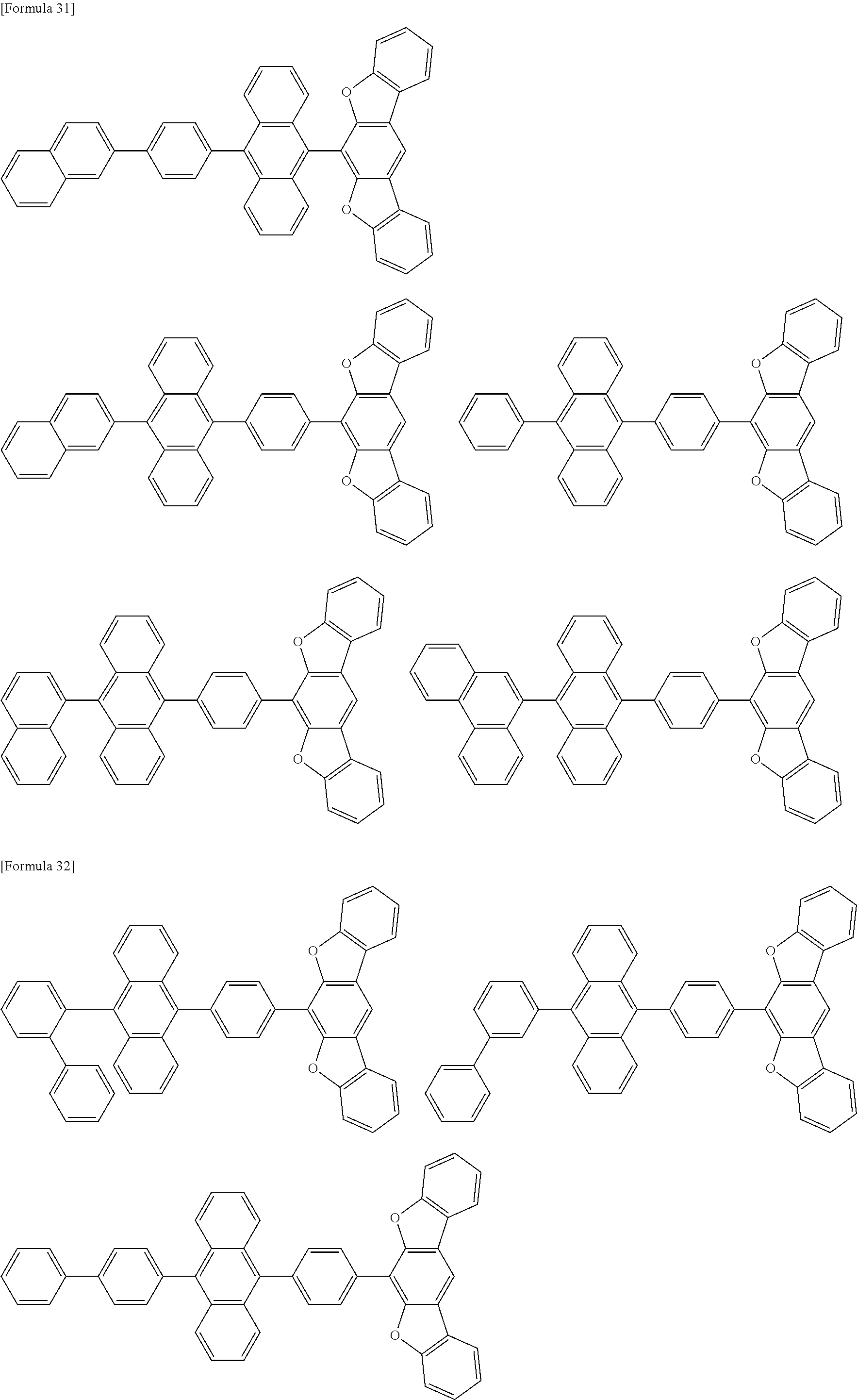

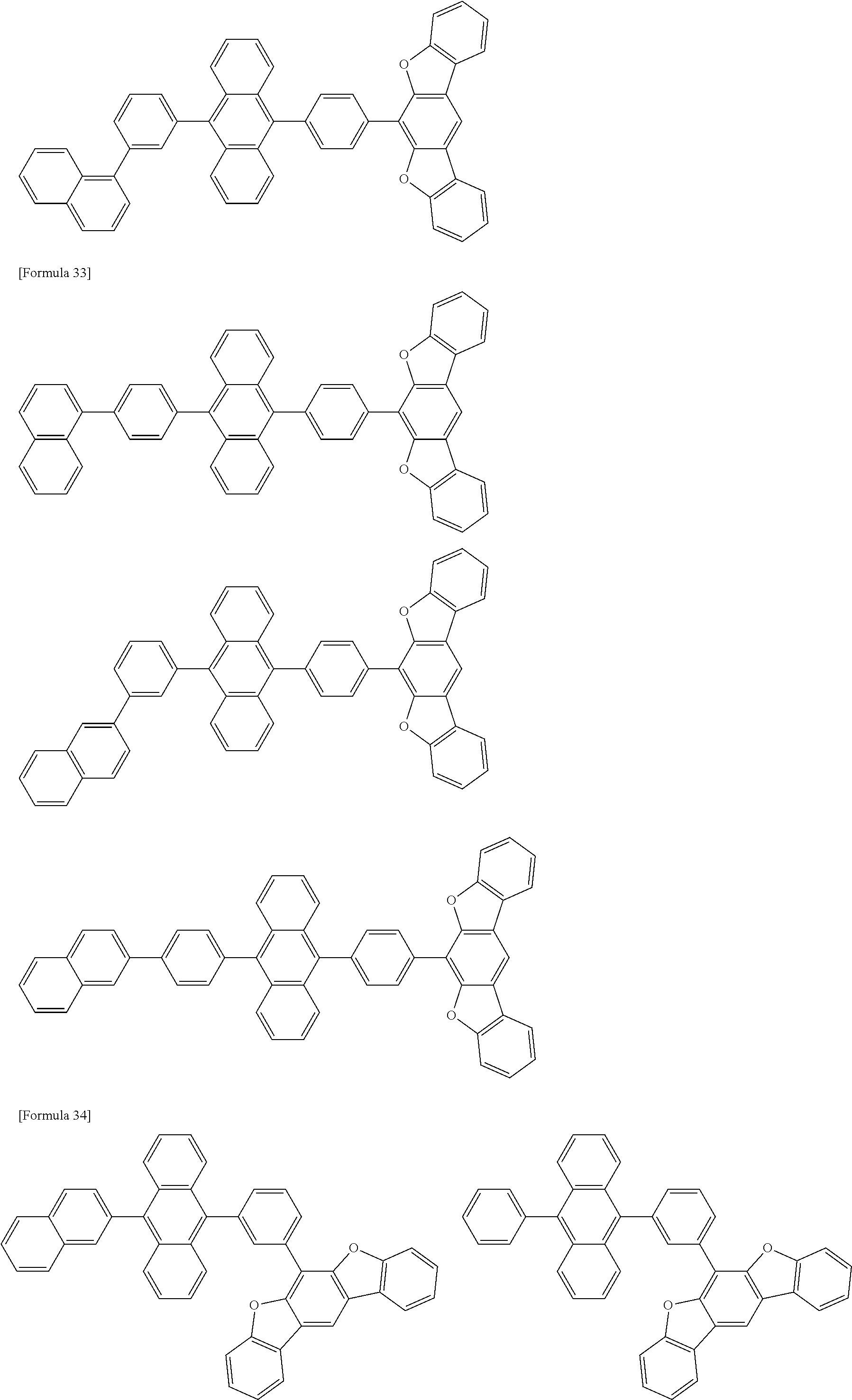

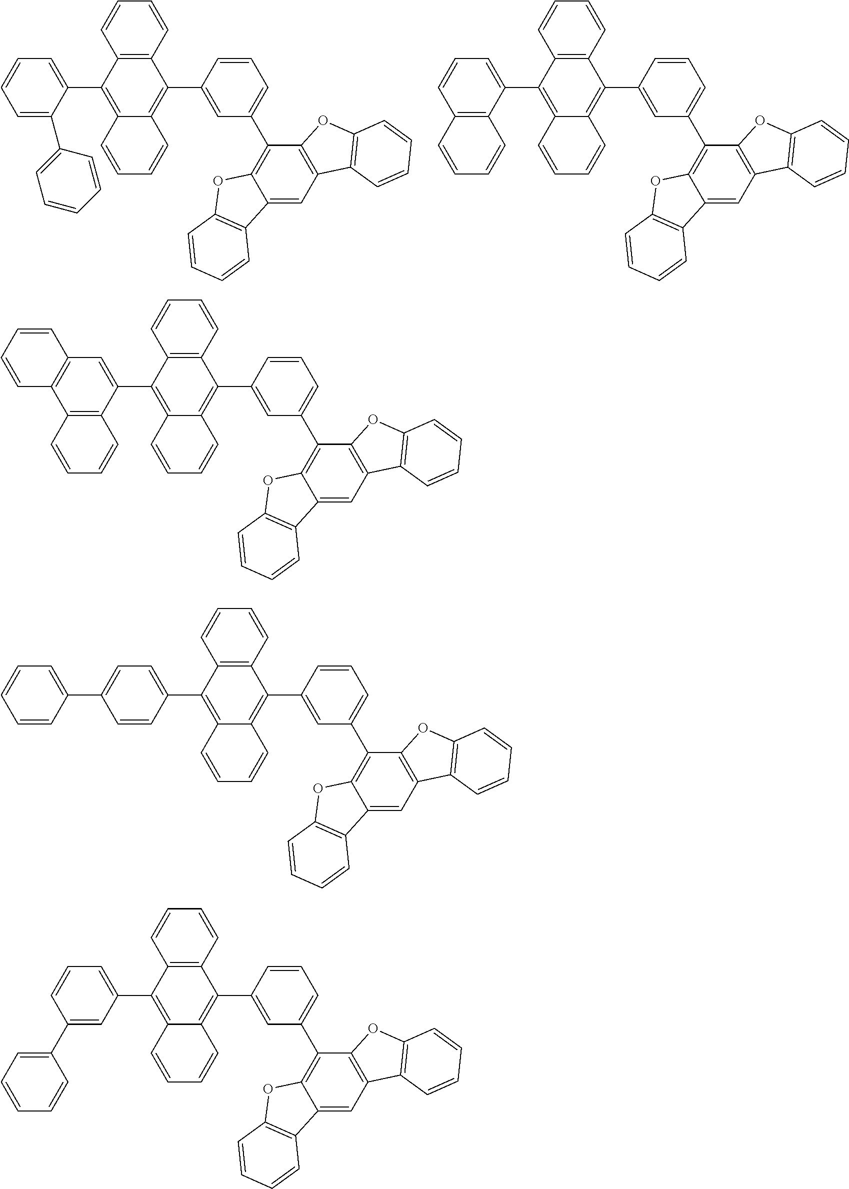

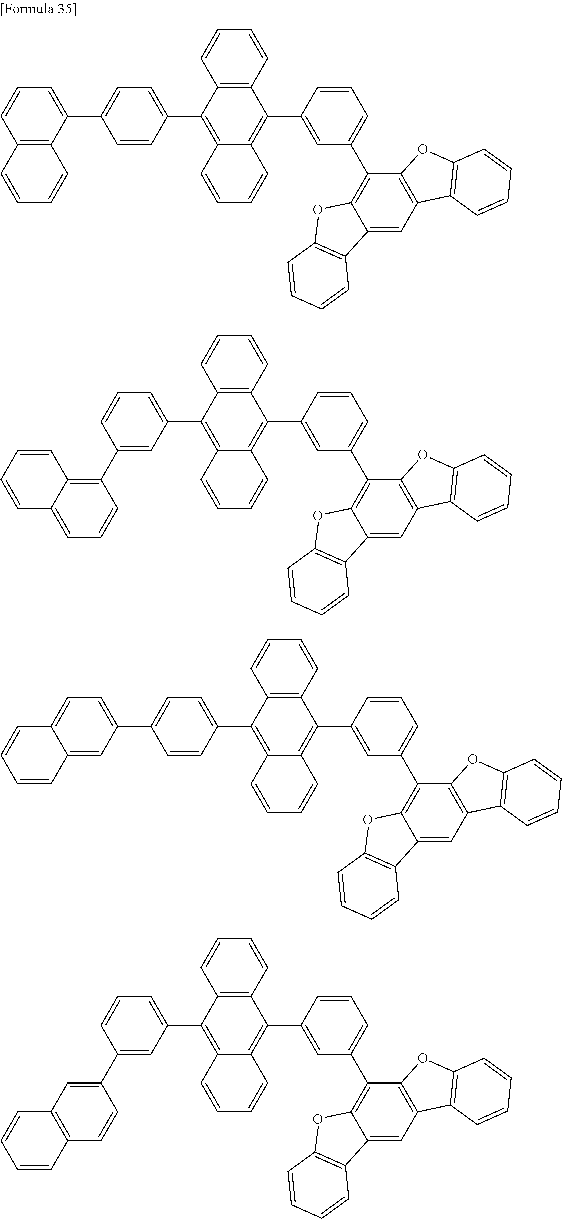

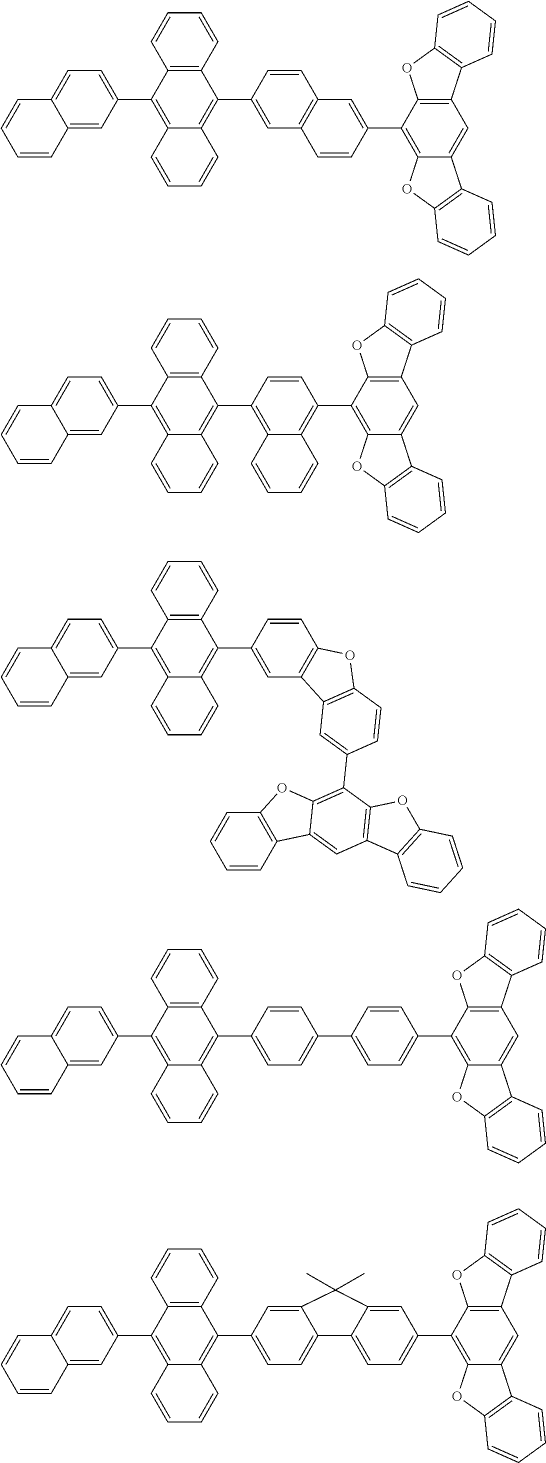

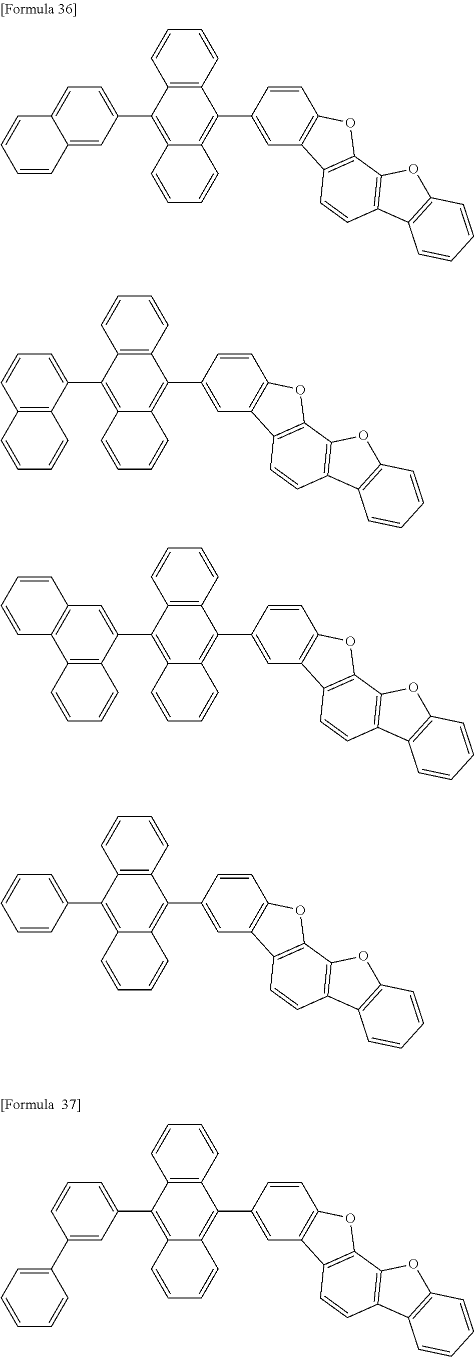 [SMILES: c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1ccc2c(c1)cc(-c1c3ccccc3c(-c3ccc4oc5c(ccc6c7ccccc7oc65)c4c3)c3ccccc13)c1ccccc12 ; c1ccc(-c2c3ccccc3c(-c3ccc4oc5c(ccc6c7ccccc7oc65)c4c3)c3ccccc23)cc1 ; c1ccc(-c2cccc(-c3c4ccccc4c(-c4ccc5oc6c(ccc7c8ccccc8oc76)c5c4)c4ccccc34)c2)cc1]

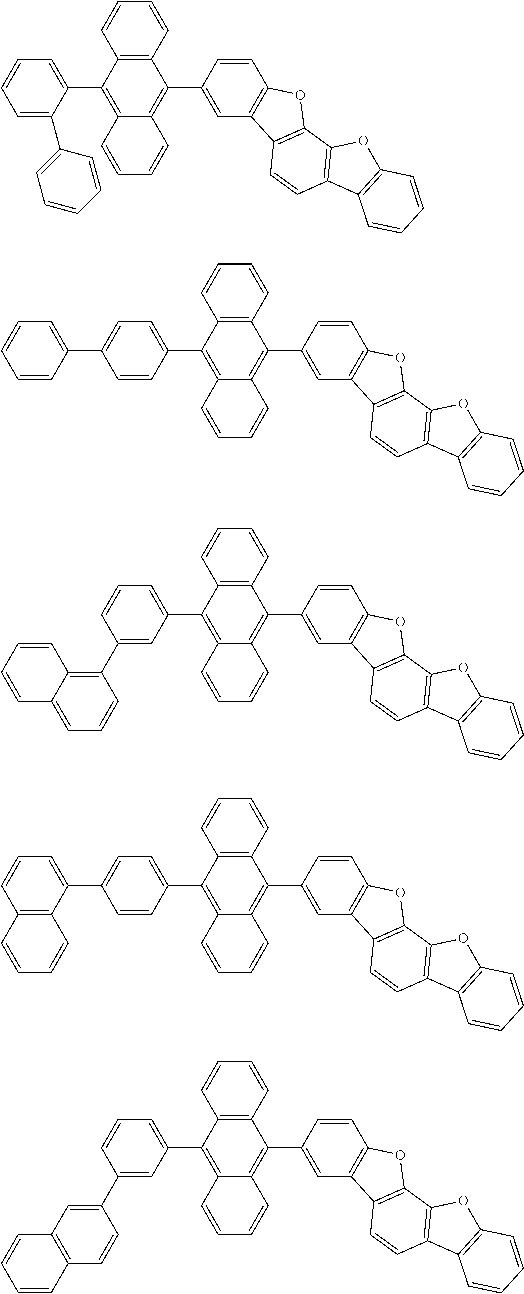 [SMILES: c1ccc(-c2ccccc2-c2c3ccccc3c(-c3ccc4oc5c(ccc6c7ccccc7oc65)c4c3)c3ccccc23)cc1 ; c1ccc(-c2ccc(-c3c4ccccc4c(-c4ccc5oc6c(ccc7c8ccccc8oc76)c5c4)c4ccccc34)cc2)cc1 ; c1cc(-c2cccc3ccccc23)cc(-c2c3ccccc3c(-c3ccc4oc5c(ccc6c7ccccc7oc65)c4c3)c3ccccc23)c1 ; c1ccc2c(-c3ccc(-c4c5ccccc5c(-c5ccc6oc7c(ccc8c9ccccc9oc87)c6c5)c5ccccc45)cc3)cccc2c1 ; c1cc(-c2ccc3ccccc3c2)cc(-c2c3ccccc3c(-c3ccc4oc5c(ccc6c7ccccc7oc65)c4c3)c3ccccc23)c1]

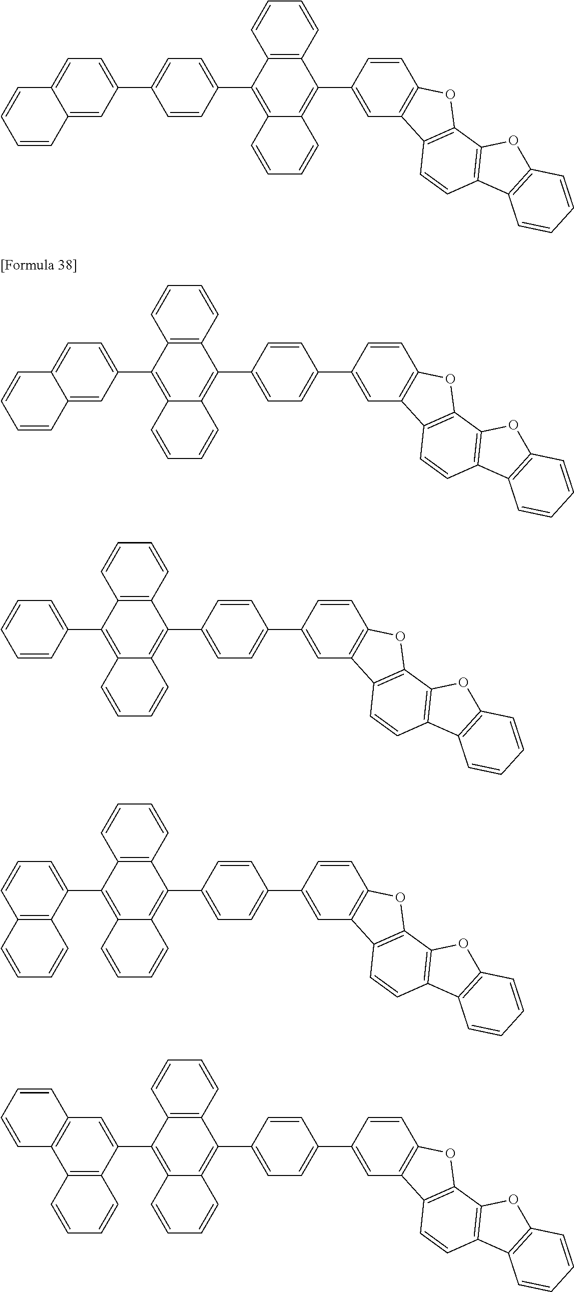 [SMILES: c1ccc2cc(-c3ccc(-c4c5ccccc5c(-c5ccc6oc7c(ccc8c9ccccc9oc87)c6c5)c5ccccc45)cc3)ccc2c1 ; c1ccc2cc(-c3c4ccccc4c(-c4ccc(-c5ccc6oc7c(ccc8c9ccccc9oc87)c6c5)cc4)c4ccccc34)ccc2c1 ; c1ccc(-c2c3ccccc3c(-c3ccc(-c4ccc5oc6c(ccc7c8ccccc8oc76)c5c4)cc3)c3ccccc23)cc1 ; c1ccc2c(-c3c4ccccc4c(-c4ccc(-c5ccc6oc7c(ccc8c9ccccc9oc87)c6c5)cc4)c4ccccc34)cccc2c1 ; c1ccc2c(c1)cc(-c1c3ccccc3c(-c3ccc(-c4ccc5oc6c(ccc7c8ccccc8oc76)c5c4)cc3)c3ccccc13)c1ccccc12]

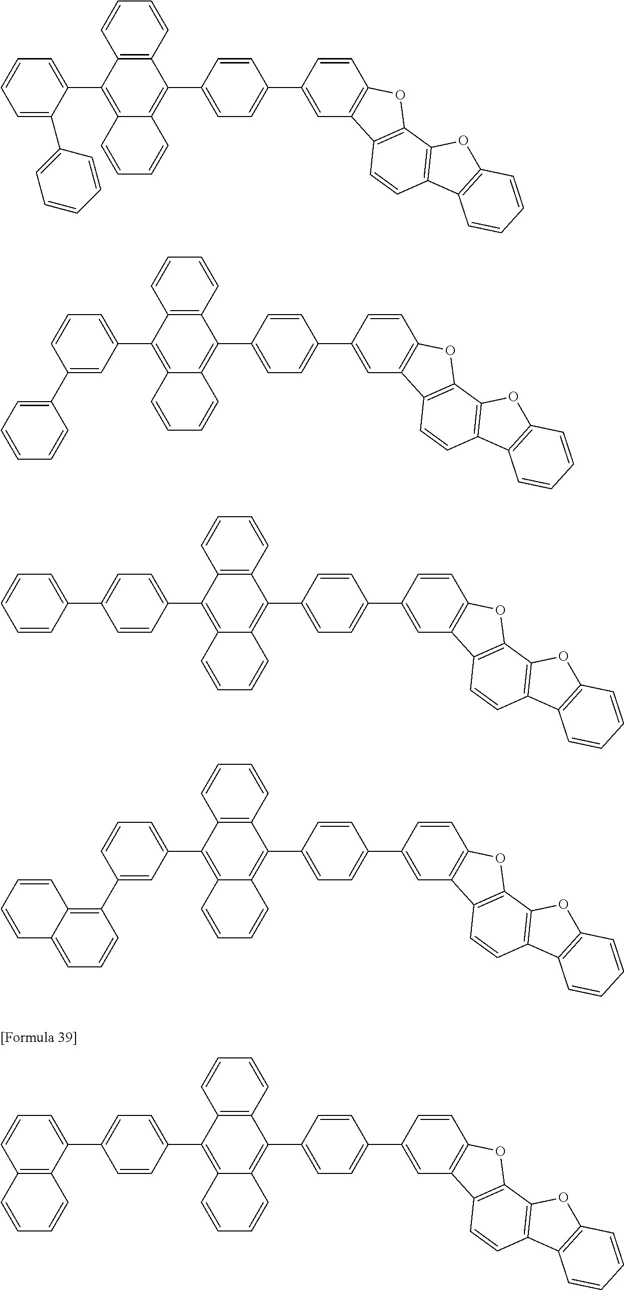 [SMILES: c1ccc(-c2ccccc2-c2c3ccccc3c(-c3ccc(-c4ccc5oc6c(ccc7c8ccccc8oc76)c5c4)cc3)c3ccccc23)cc1 ; c1ccc(-c2cccc(-c3c4ccccc4c(-c4ccc(-c5ccc6oc7c(ccc8c9ccccc9oc87)c6c5)cc4)c4ccccc34)c2)cc1 ; c1ccc(-c2ccc(-c3c4ccccc4c(-c4ccc(-c5ccc6oc7c(ccc8c9ccccc9oc87)c6c5)cc4)c4ccccc34)cc2)cc1 ; c1cc(-c2cccc3ccccc23)cc(-c2c3ccccc3c(-c3ccc(-c4ccc5oc6c(ccc7c8ccccc8oc76)c5c4)cc3)c3ccccc23)c1 ; c1ccc2c(-c3ccc(-c4c5ccccc5c(-c5ccc(-c6ccc7oc8c(ccc9c%10ccccc%10oc98)c7c6)cc5)c5ccccc45)cc3)cccc2c1]

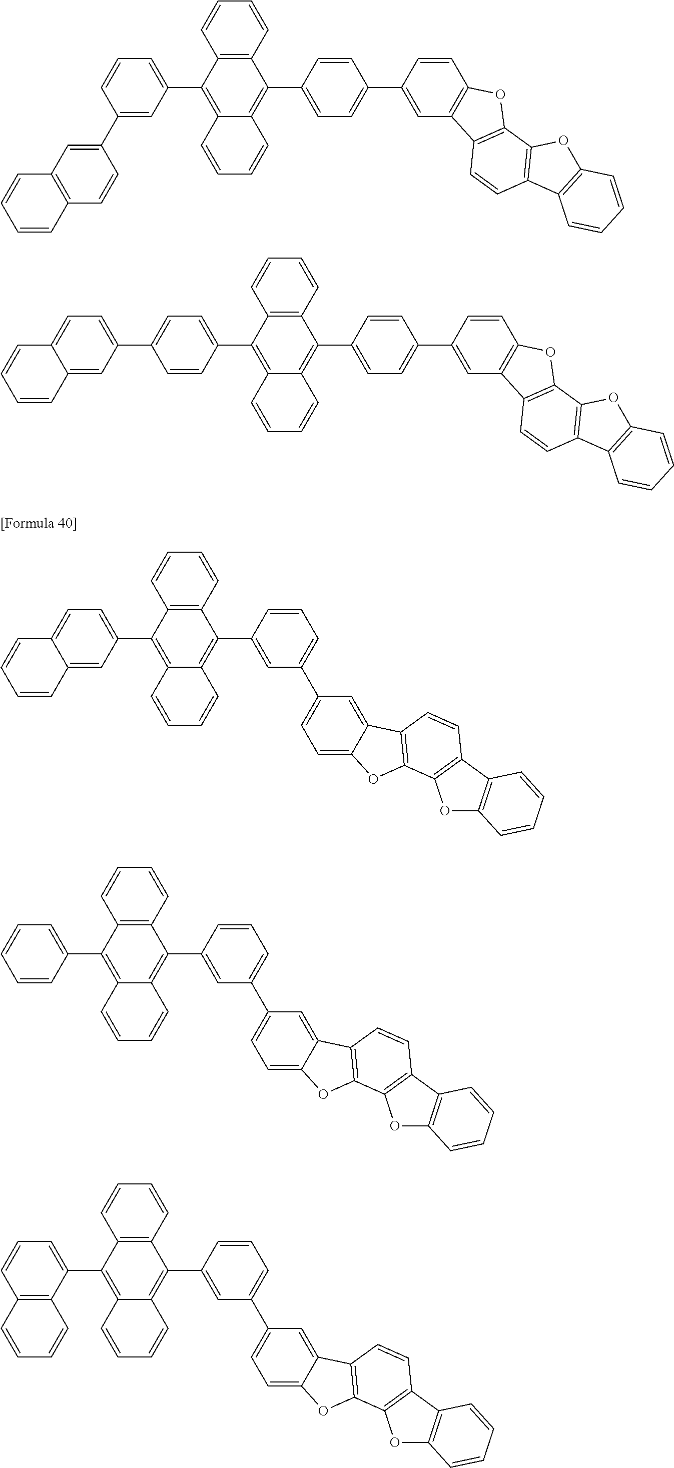 [SMILES: c1cc(-c2ccc3ccccc3c2)cc(-c2c3ccccc3c(-c3ccc(-c4ccc5oc6c(ccc7c8ccccc8oc76)c5c4)cc3)c3ccccc23)c1 ; c1ccc2cc(-c3ccc(-c4c5ccccc5c(-c5ccc(-c6ccc7oc8c(ccc9c%10ccccc%10oc98)c7c6)cc5)c5ccccc45)cc3)ccc2c1 ; c1cc(-c2ccc3oc4c(ccc5c6ccccc6oc54)c3c2)cc(-c2c3ccccc3c(-c3ccc4ccccc4c3)c3ccccc23)c1 ; c1ccc(-c2c3ccccc3c(-c3cccc(-c4ccc5oc6c(ccc7c8ccccc8oc76)c5c4)c3)c3ccccc23)cc1 ; c1cc(-c2ccc3oc4c(ccc5c6ccccc6oc54)c3c2)cc(-c2c3ccccc3c(-c3cccc4ccccc34)c3ccccc23)c1]

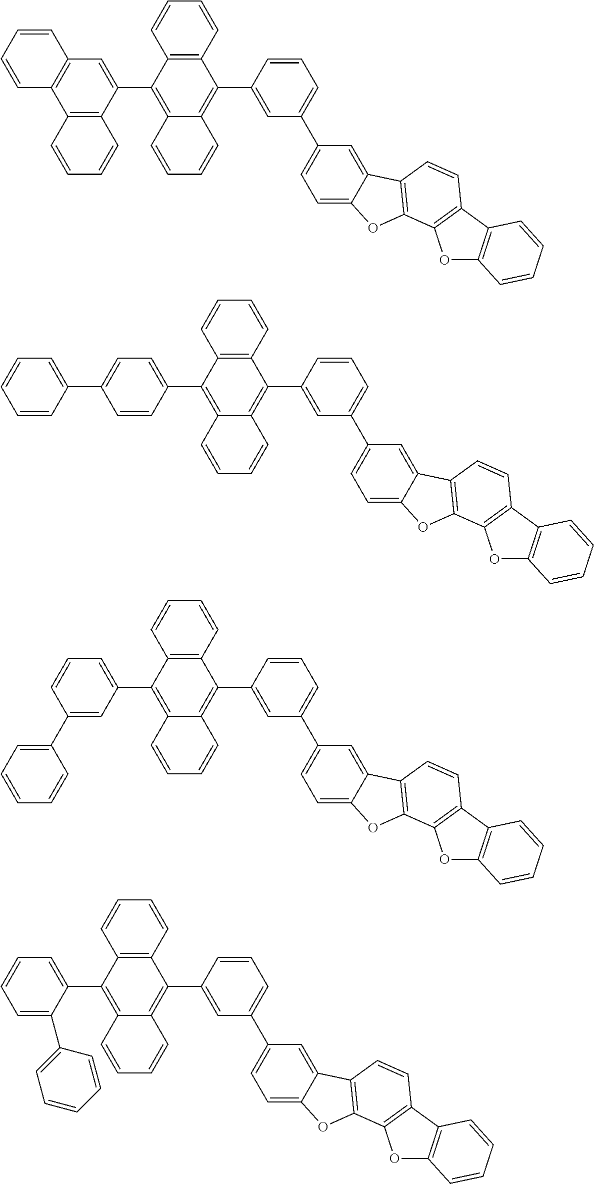 [SMILES: c1cc(-c2ccc3oc4c(ccc5c6ccccc6oc54)c3c2)cc(-c2c3ccccc3c(-c3cc4ccccc4c4ccccc34)c3ccccc23)c1 ; c1ccc(-c2ccc(-c3c4ccccc4c(-c4cccc(-c5ccc6oc7c(ccc8c9ccccc9oc87)c6c5)c4)c4ccccc34)cc2)cc1 ; c1ccc(-c2cccc(-c3c4ccccc4c(-c4cccc(-c5ccc6oc7c(ccc8c9ccccc9oc87)c6c5)c4)c4ccccc34)c2)cc1 ; c1ccc(-c2ccccc2-c2c3ccccc3c(-c3cccc(-c4ccc5oc6c(ccc7c8ccccc8oc76)c5c4)c3)c3ccccc23)cc1]

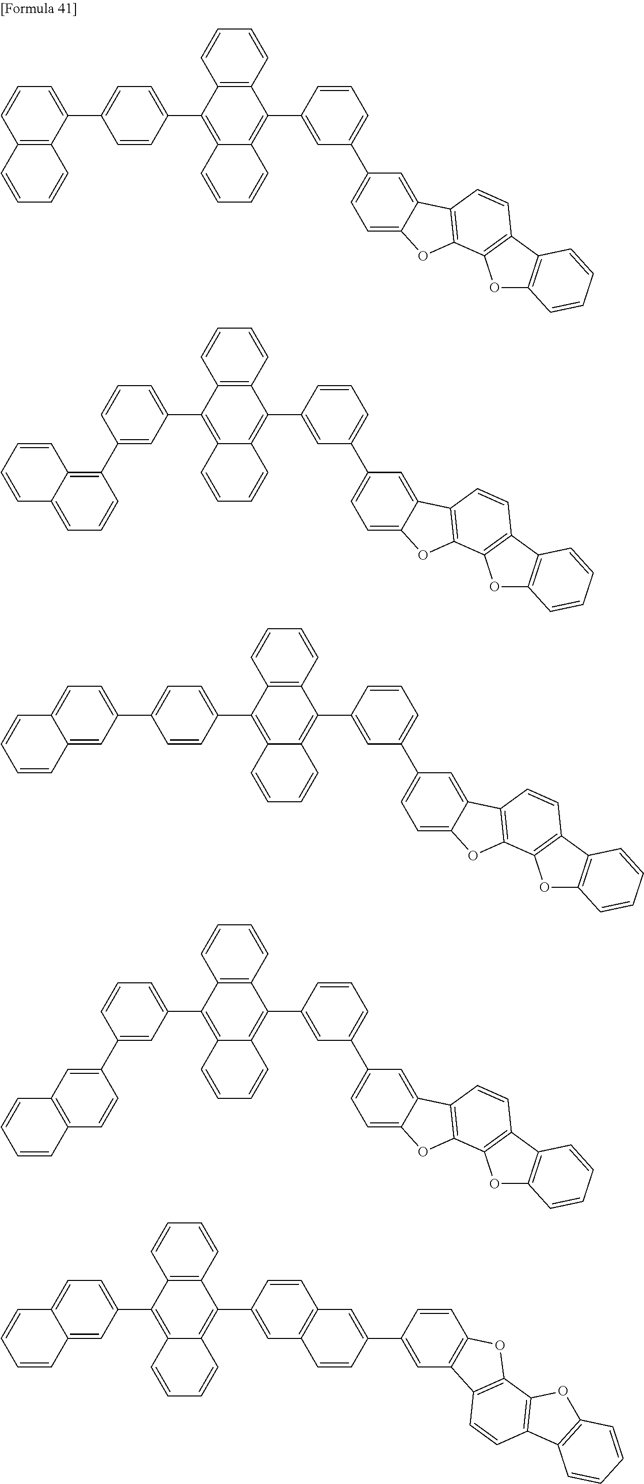 [SMILES: c1cc(-c2ccc3oc4c(ccc5c6ccccc6oc54)c3c2)cc(-c2c3ccccc3c(-c3ccc(-c4cccc5ccccc45)cc3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2ccc3oc4c(ccc5c6ccccc6oc54)c3c2)cc(-c2c3ccccc3c(-c3ccc(-c4ccc5ccccc5c4)cc3)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1ccc2cc(-c3c4ccccc4c(-c4ccc5cc(-c6ccc7oc8c(ccc9c%10ccccc%10oc98)c7c6)ccc5c4)c4ccccc34)ccc2c1]

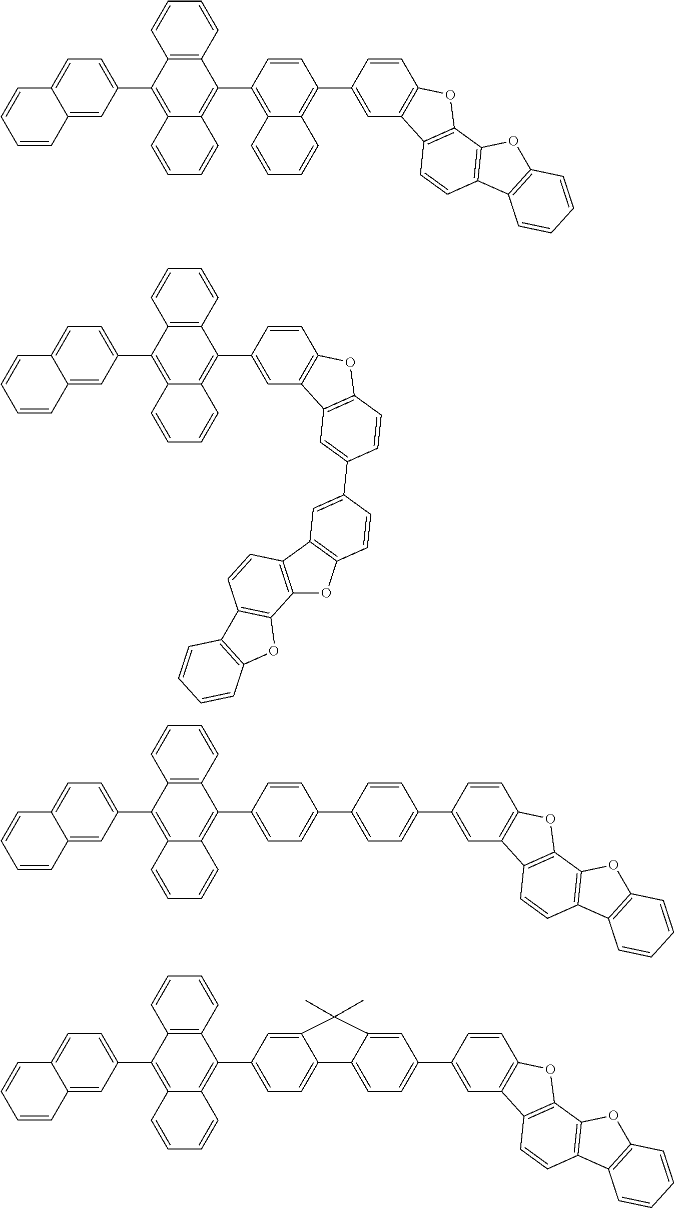

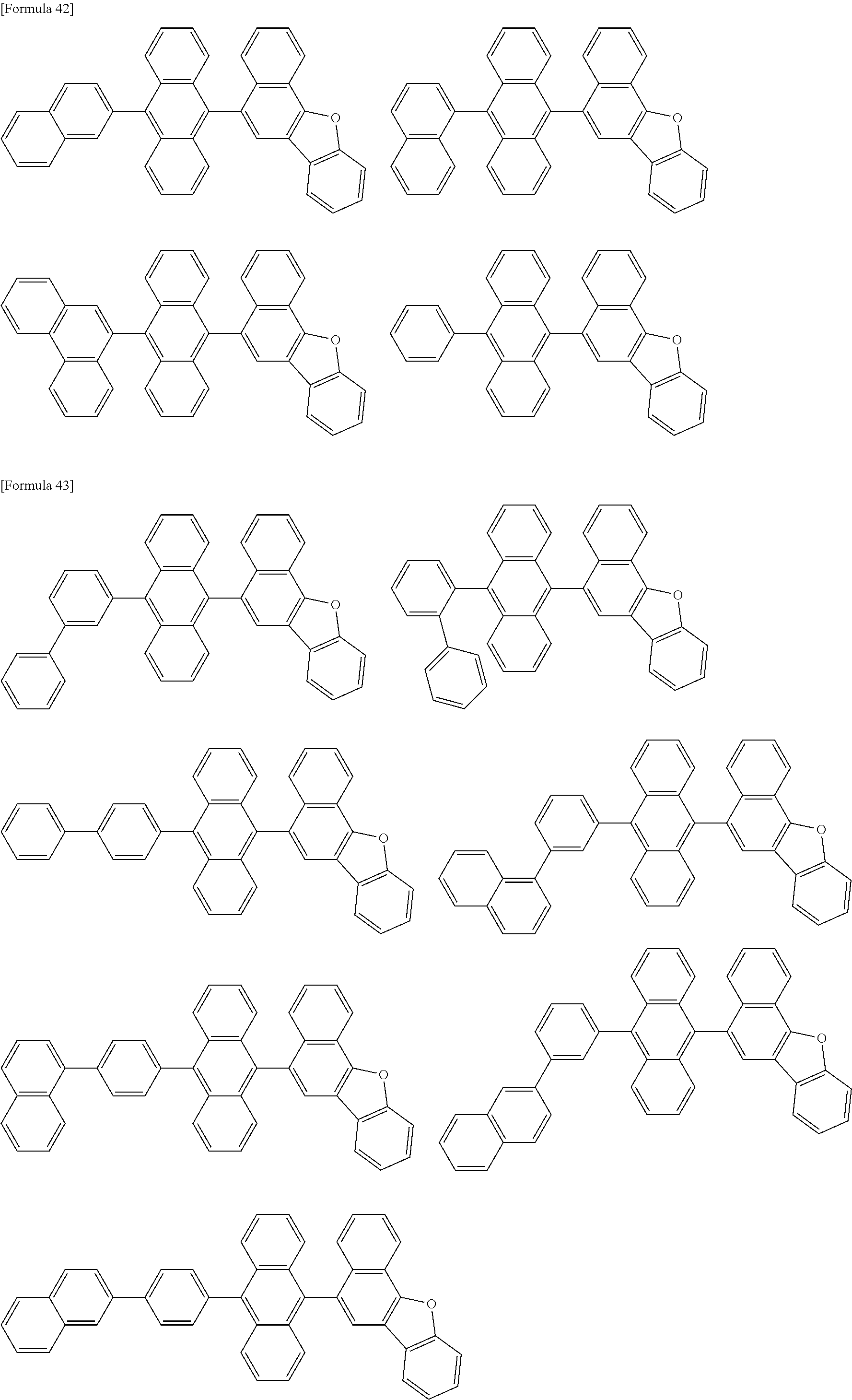

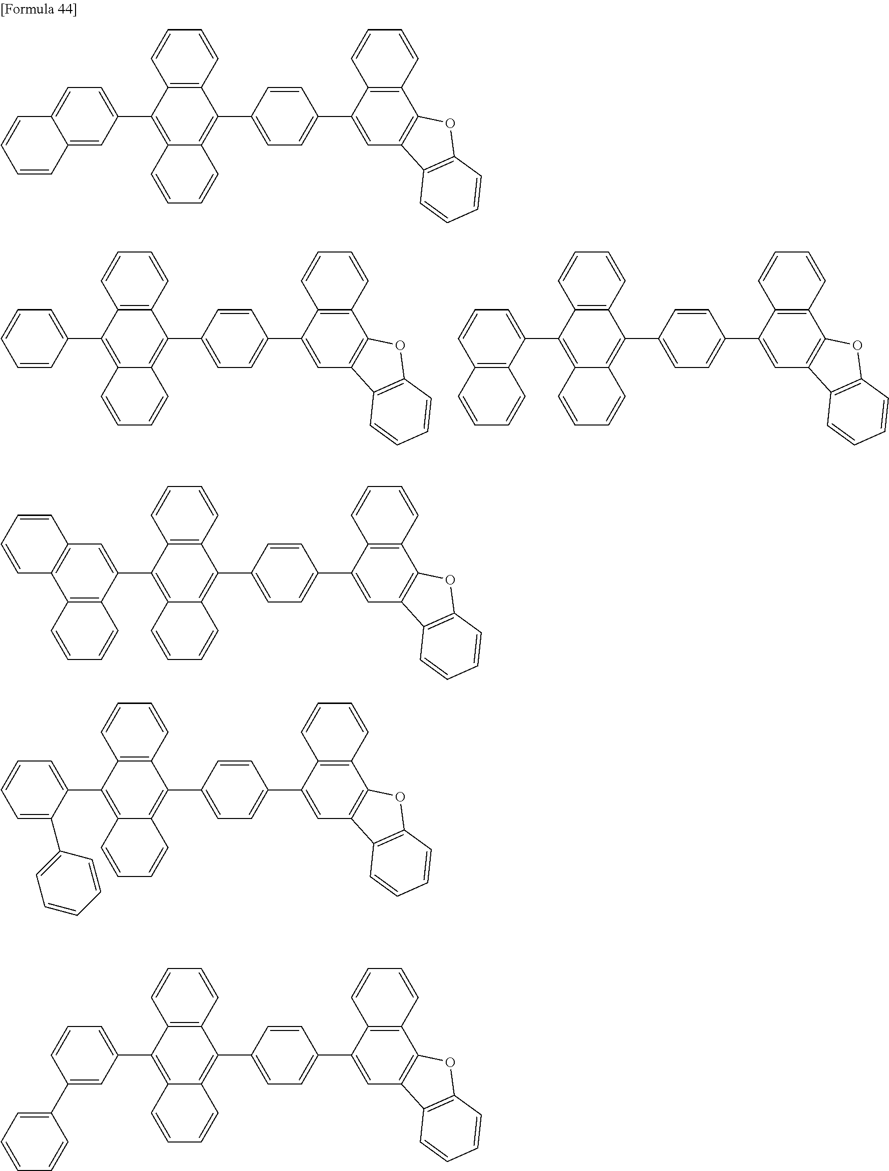

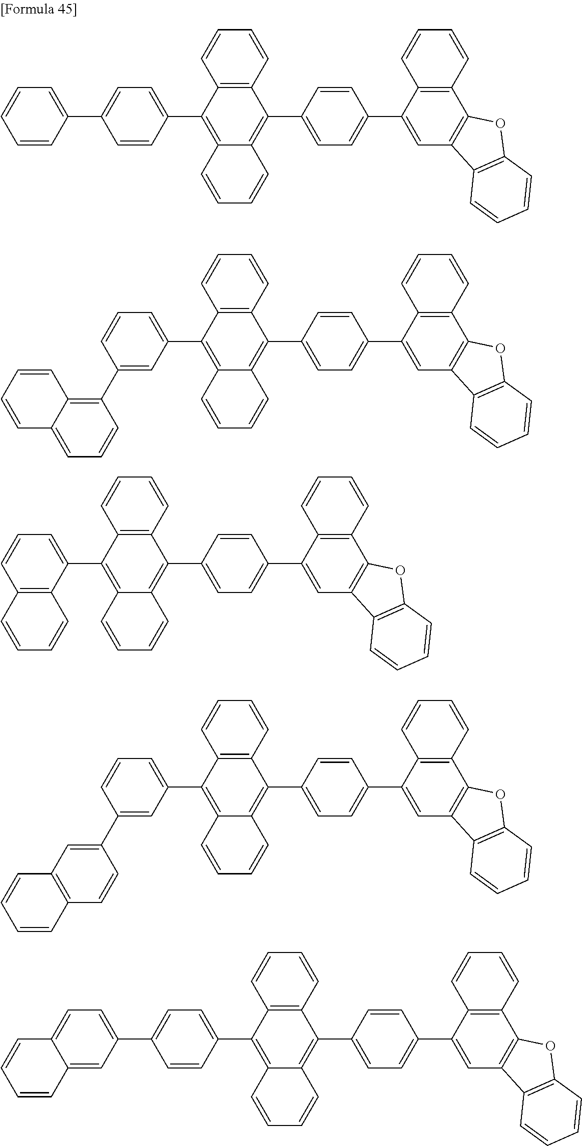

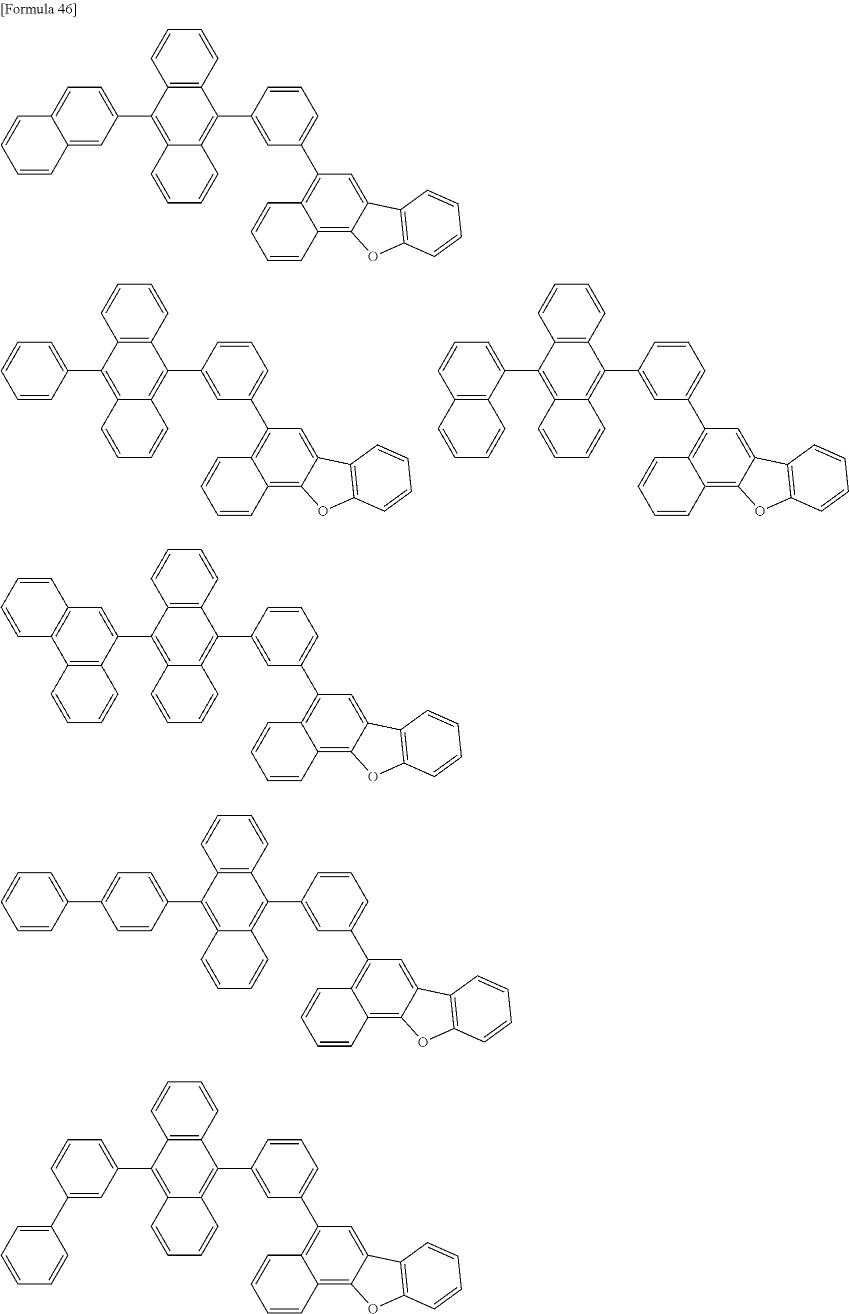

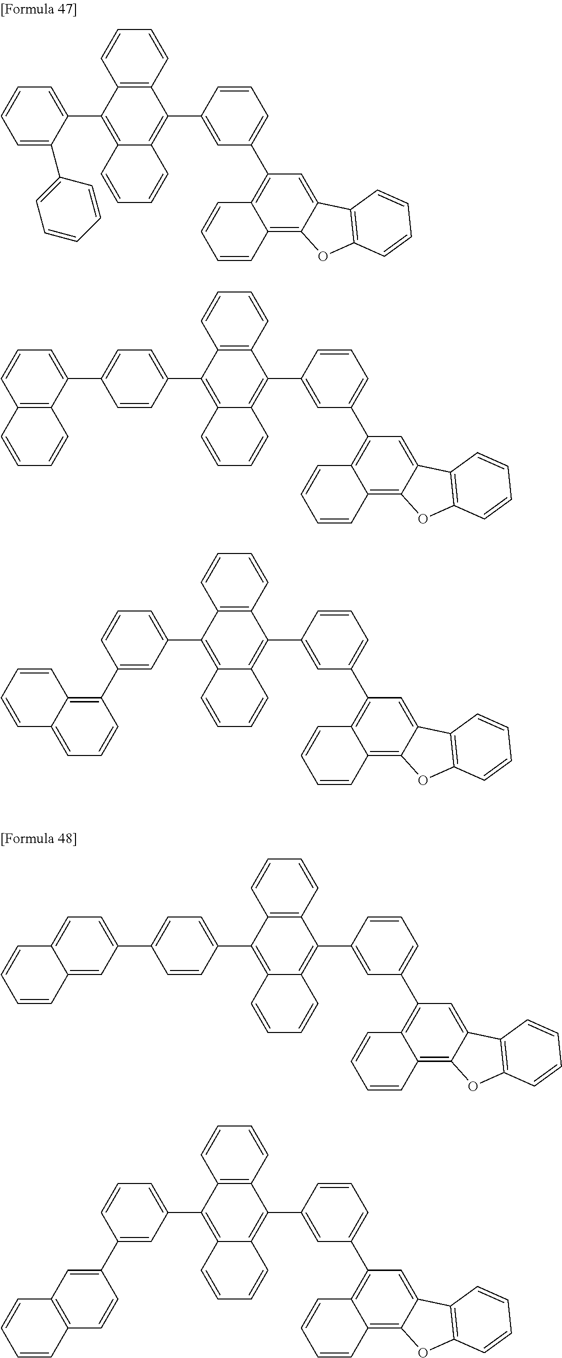

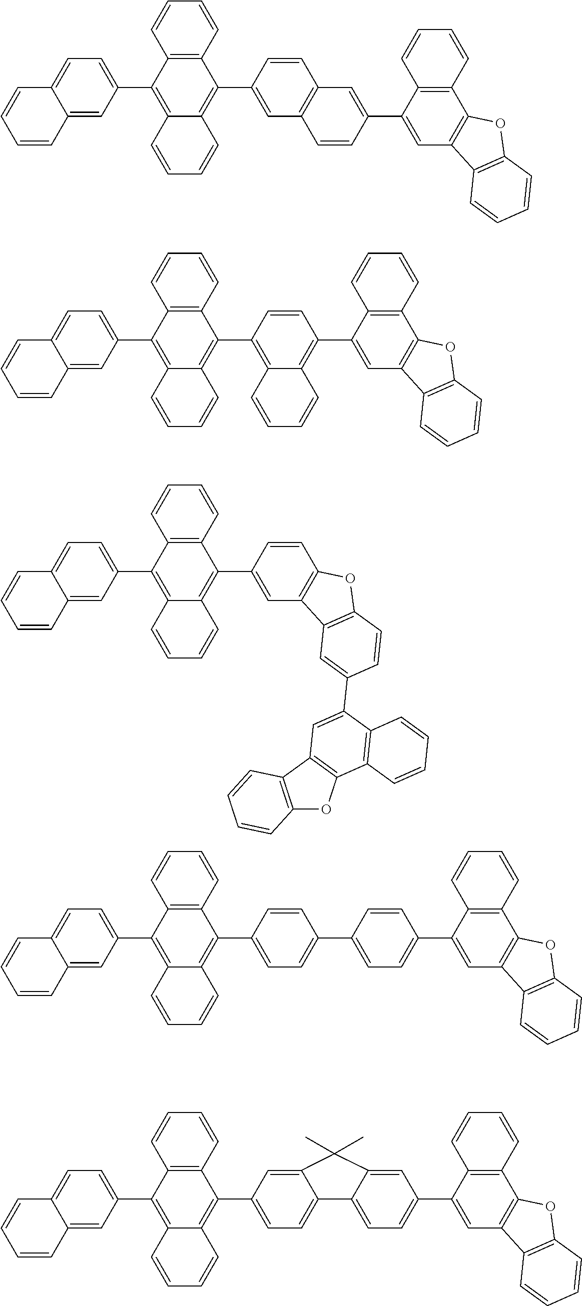

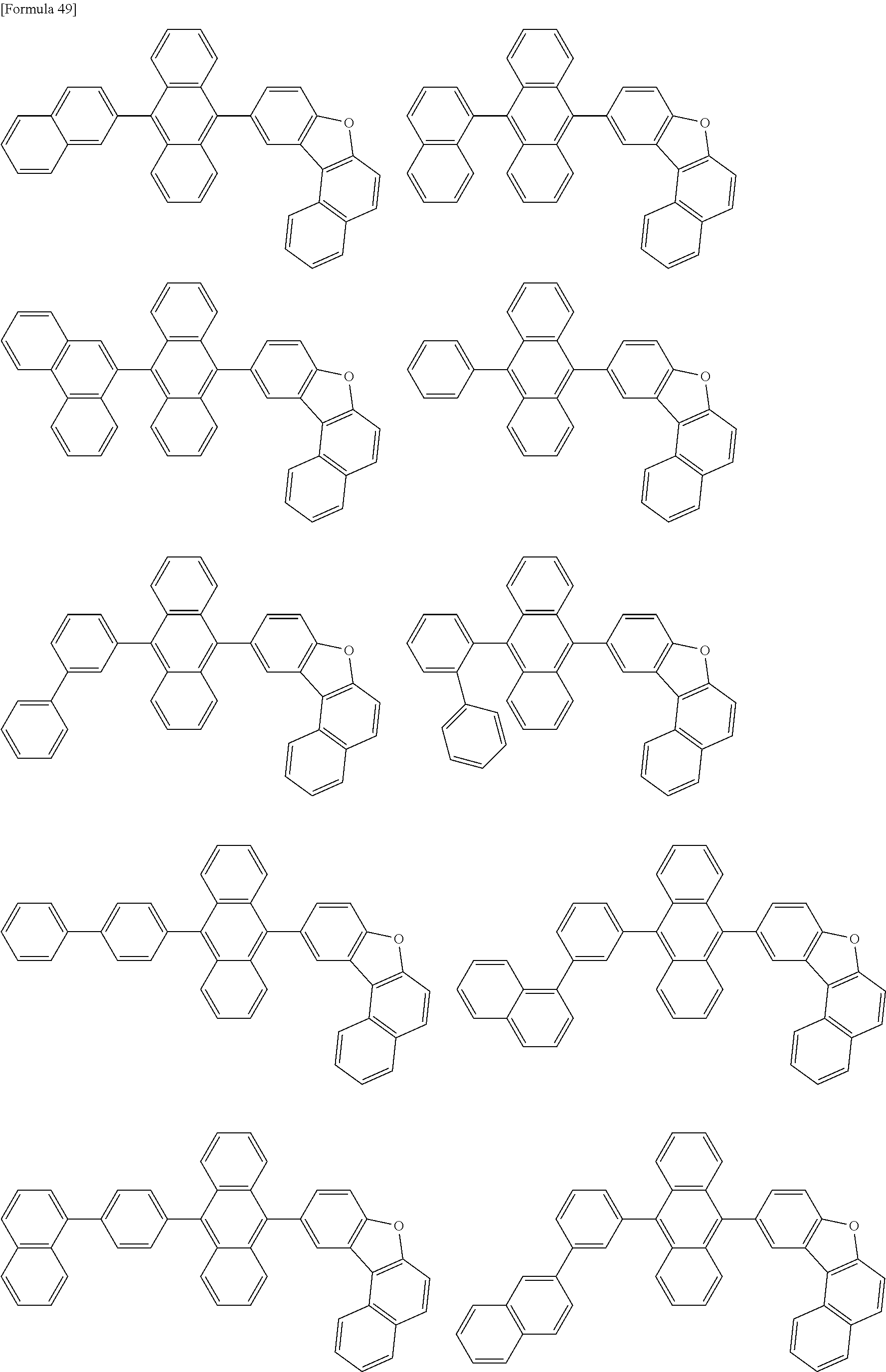

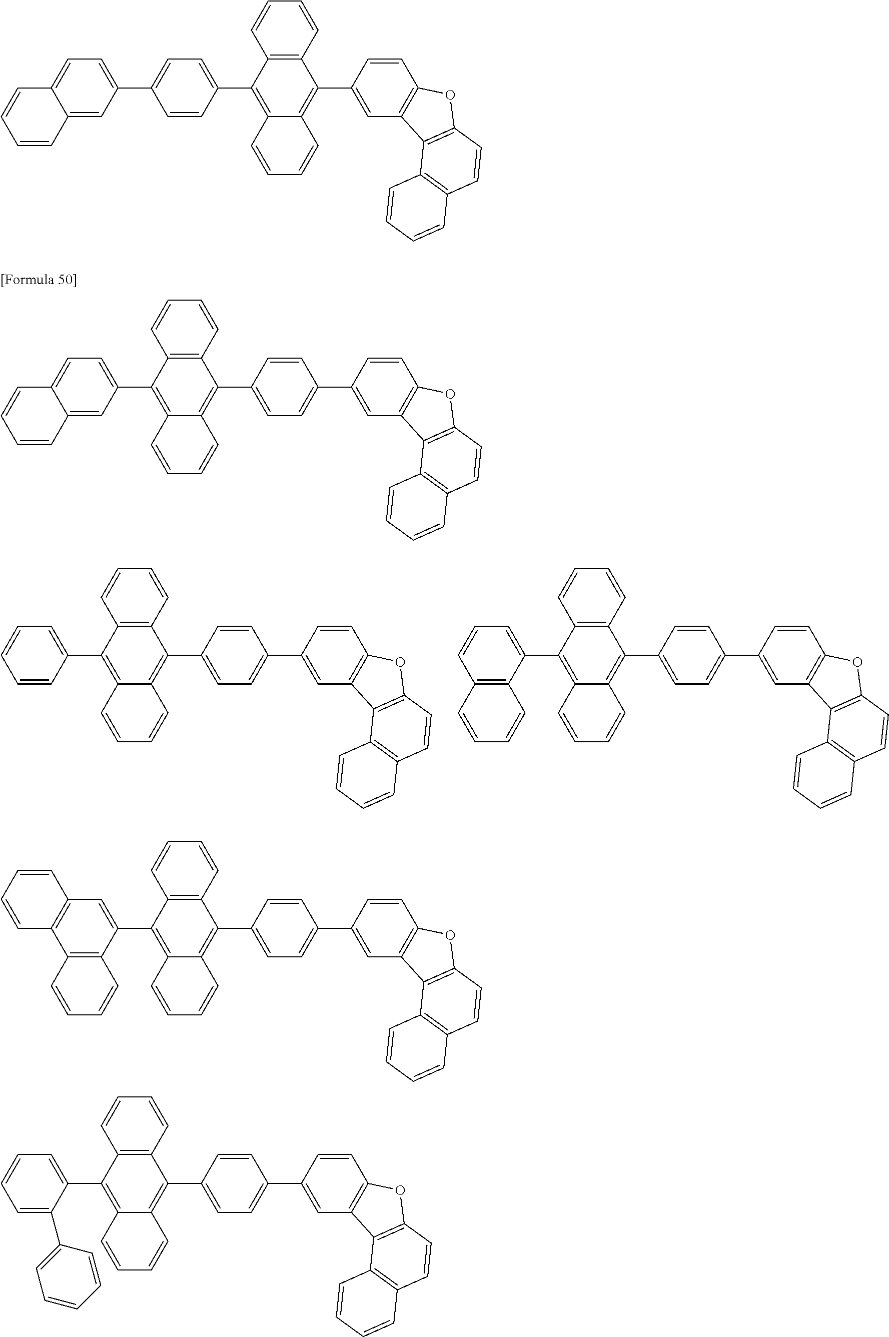

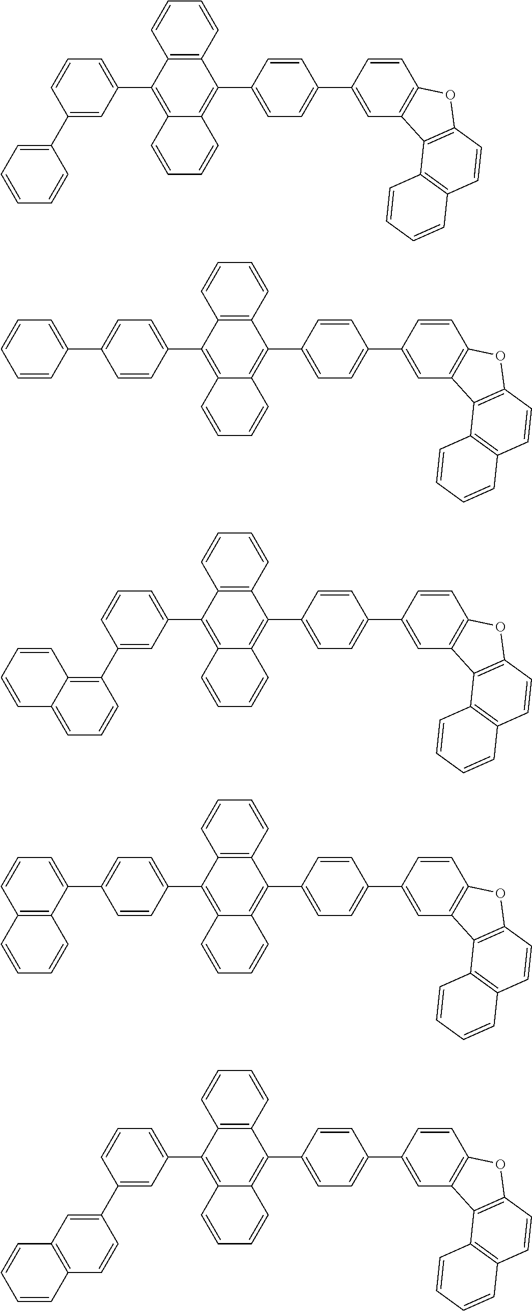 [SMILES: c1ccc(-c2cccc(-c3c4ccccc4c(-c4ccc(-c5ccc6oc7ccc8ccccc8c7c6c5)cc4)c4ccccc34)c2)cc1 ; c1ccc(-c2ccc(-c3c4ccccc4c(-c4ccc(-c5ccc6oc7ccc8ccccc8c7c6c5)cc4)c4ccccc34)cc2)cc1 ; c1cc(-c2cccc3ccccc23)cc(-c2c3ccccc3c(-c3ccc(-c4ccc5oc6ccc7ccccc7c6c5c4)cc3)c3ccccc23)c1 ; c1ccc2c(-c3ccc(-c4c5ccccc5c(-c5ccc(-c6ccc7oc8ccc9ccccc9c8c7c6)cc5)c5ccccc45)cc3)cccc2c1 ; c1cc(-c2ccc3ccccc3c2)cc(-c2c3ccccc3c(-c3ccc(-c4ccc5oc6ccc7ccccc7c6c5c4)cc3)c3ccccc23)c1]

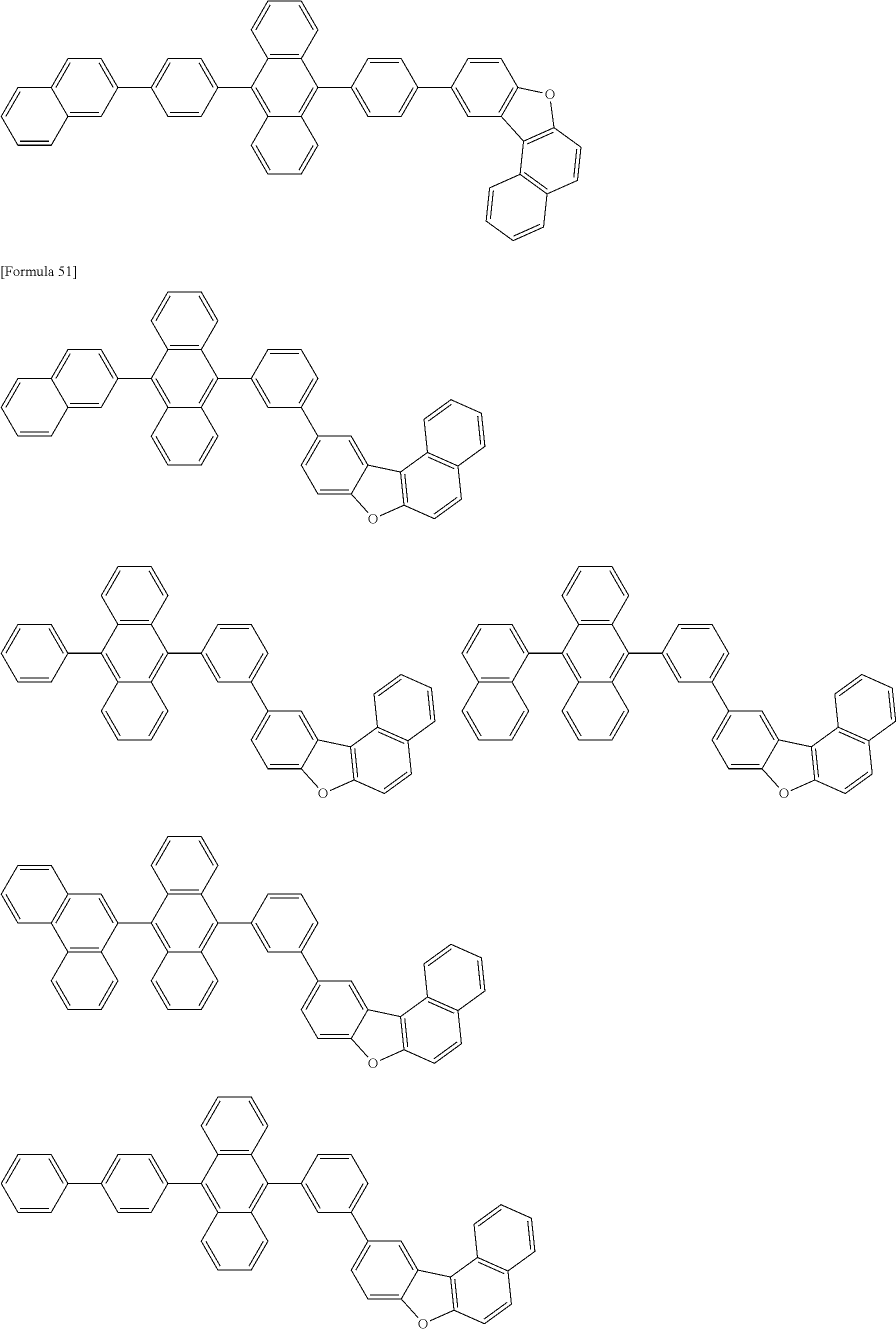

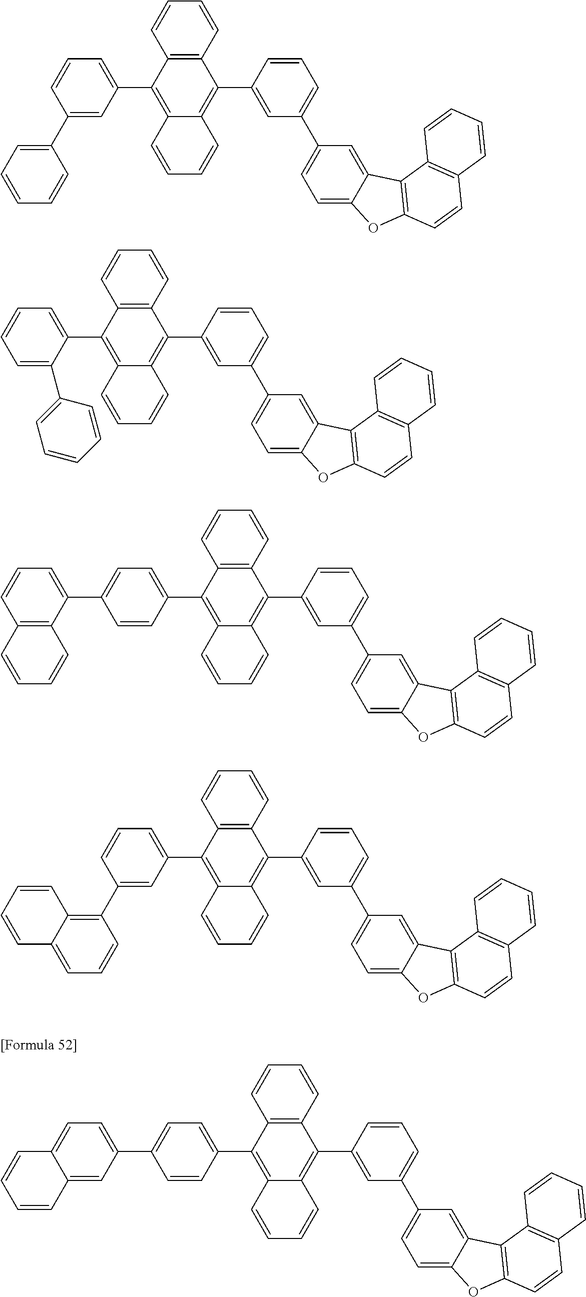 [SMILES: c1ccc(-c2cccc(-c3c4ccccc4c(-c4cccc(-c5ccc6oc7ccc8ccccc8c7c6c5)c4)c4ccccc34)c2)cc1 ; c1ccc(-c2ccccc2-c2c3ccccc3c(-c3cccc(-c4ccc5oc6ccc7ccccc7c6c5c4)c3)c3ccccc23)cc1 ; c1cc(-c2ccc3oc4ccc5ccccc5c4c3c2)cc(-c2c3ccccc3c(-c3ccc(-c4cccc5ccccc45)cc3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2ccc3oc4ccc5ccccc5c4c3c2)cc(-c2c3ccccc3c(-c3ccc(-c4ccc5ccccc5c4)cc3)c3ccccc23)c1]

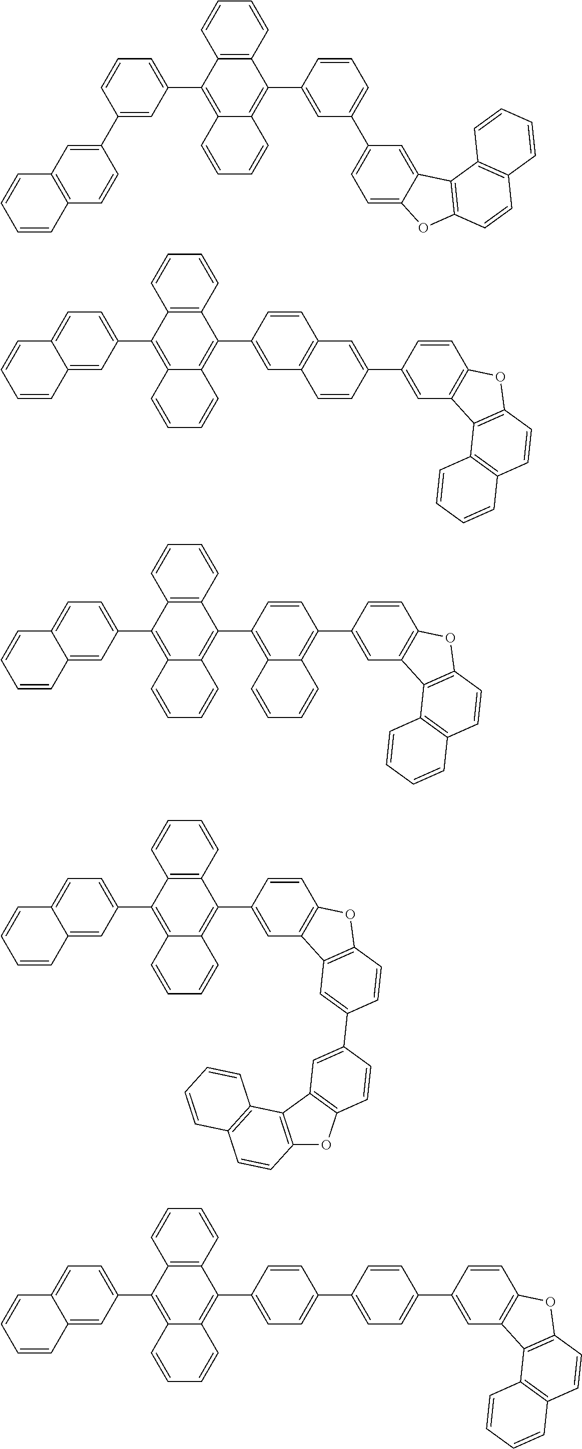 [SMILES: c1cc(-c2ccc3ccccc3c2)cc(-c2c3ccccc3c(-c3cccc(-c4ccc5oc6ccc7ccccc7c6c5c4)c3)c3ccccc23)c1 ; c1ccc2cc(-c3c4ccccc4c(-c4ccc5cc(-c6ccc7oc8ccc9ccccc9c8c7c6)ccc5c4)c4ccccc34)ccc2c1 ; c1ccc2cc(-c3c4ccccc4c(-c4ccc(-c5ccc6oc7ccc8ccccc8c7c6c5)c5ccccc45)c4ccccc34)ccc2c1 ; c1ccc2cc(-c3c4ccccc4c(-c4ccc5oc6ccc(-c7ccc8oc9ccc%10ccccc%10c9c8c7)cc6c5c4)c4ccccc34)ccc2c1 ; c1ccc2cc(-c3c4ccccc4c(-c4ccc(-c5ccc(-c6ccc7oc8ccc9ccccc9c8c7c6)cc5)cc4)c4ccccc34)ccc2c1]

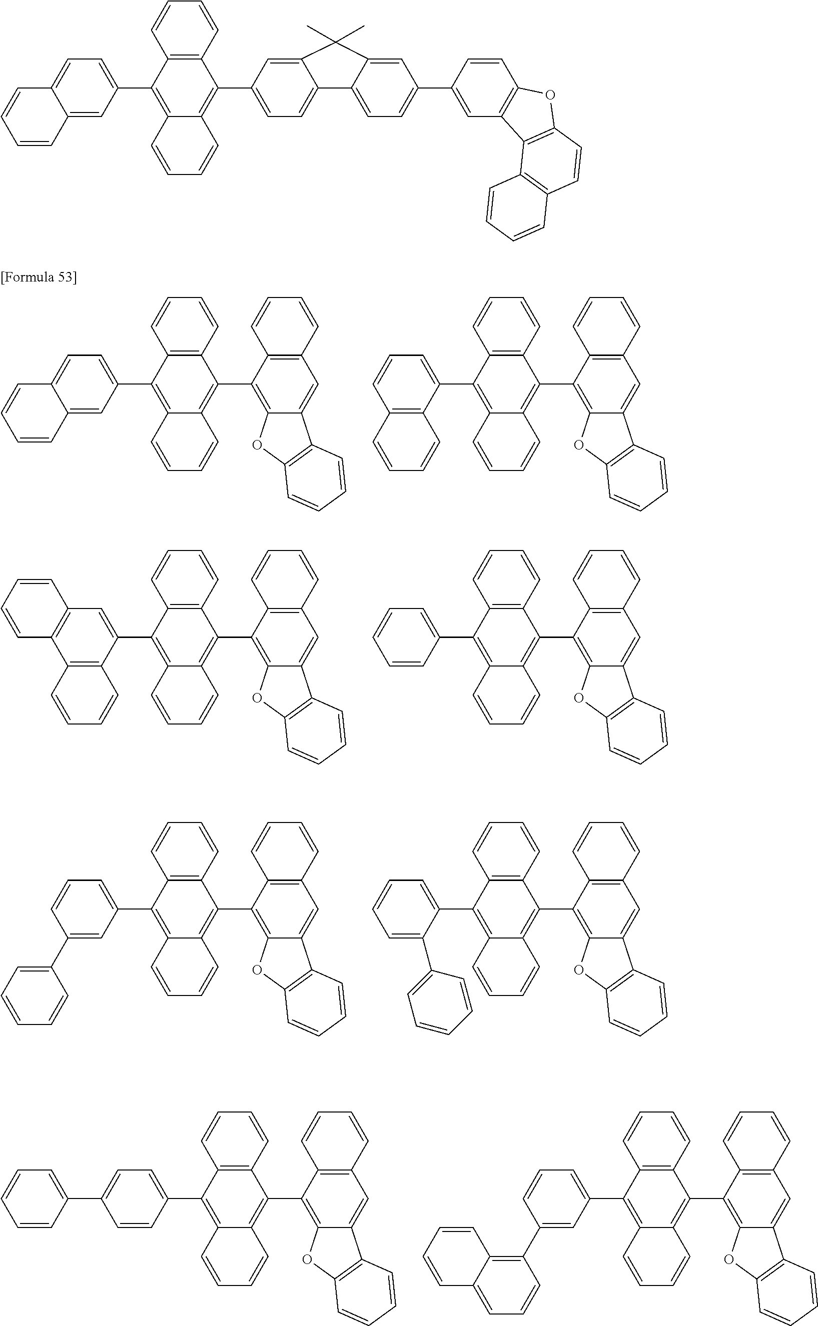

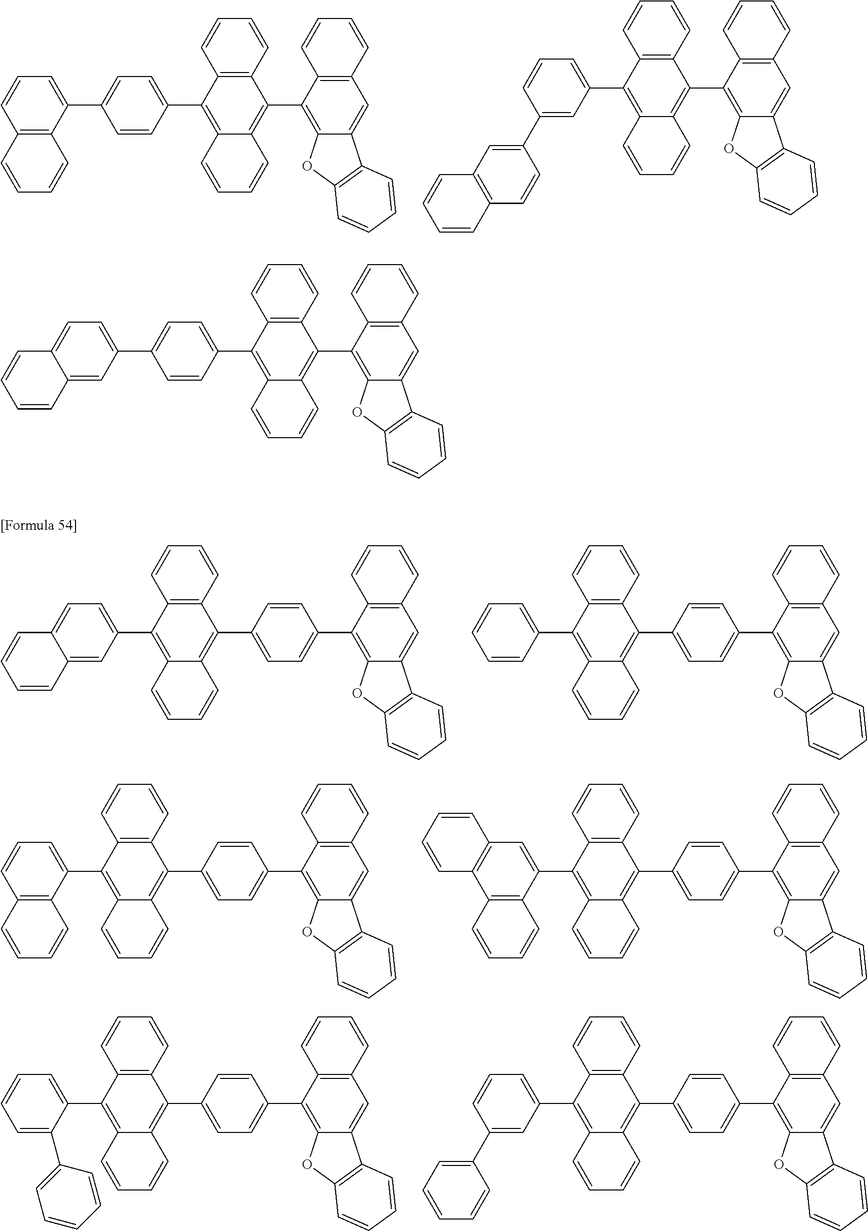

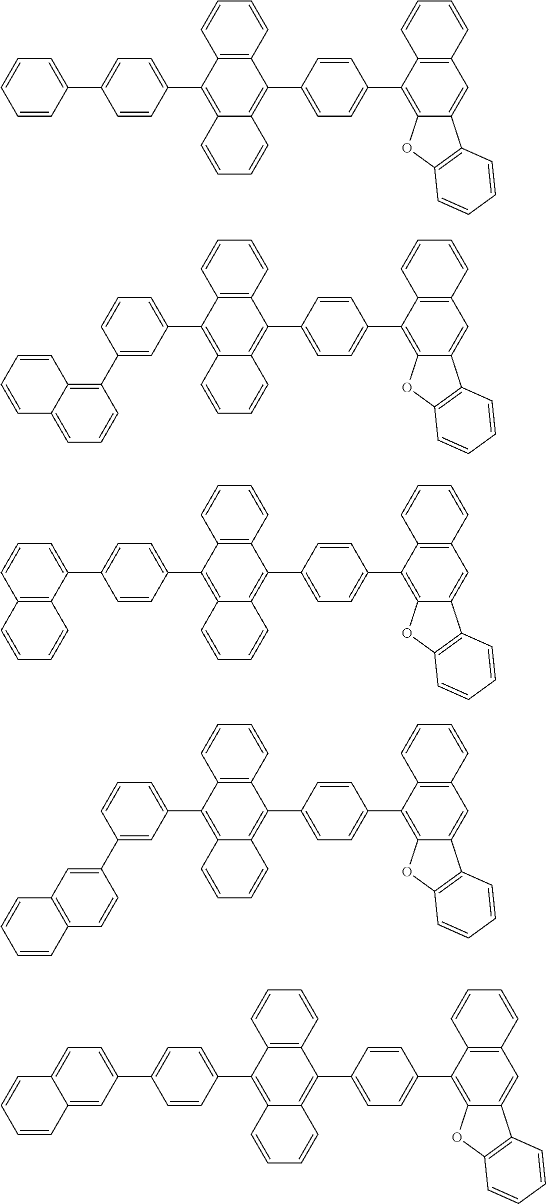

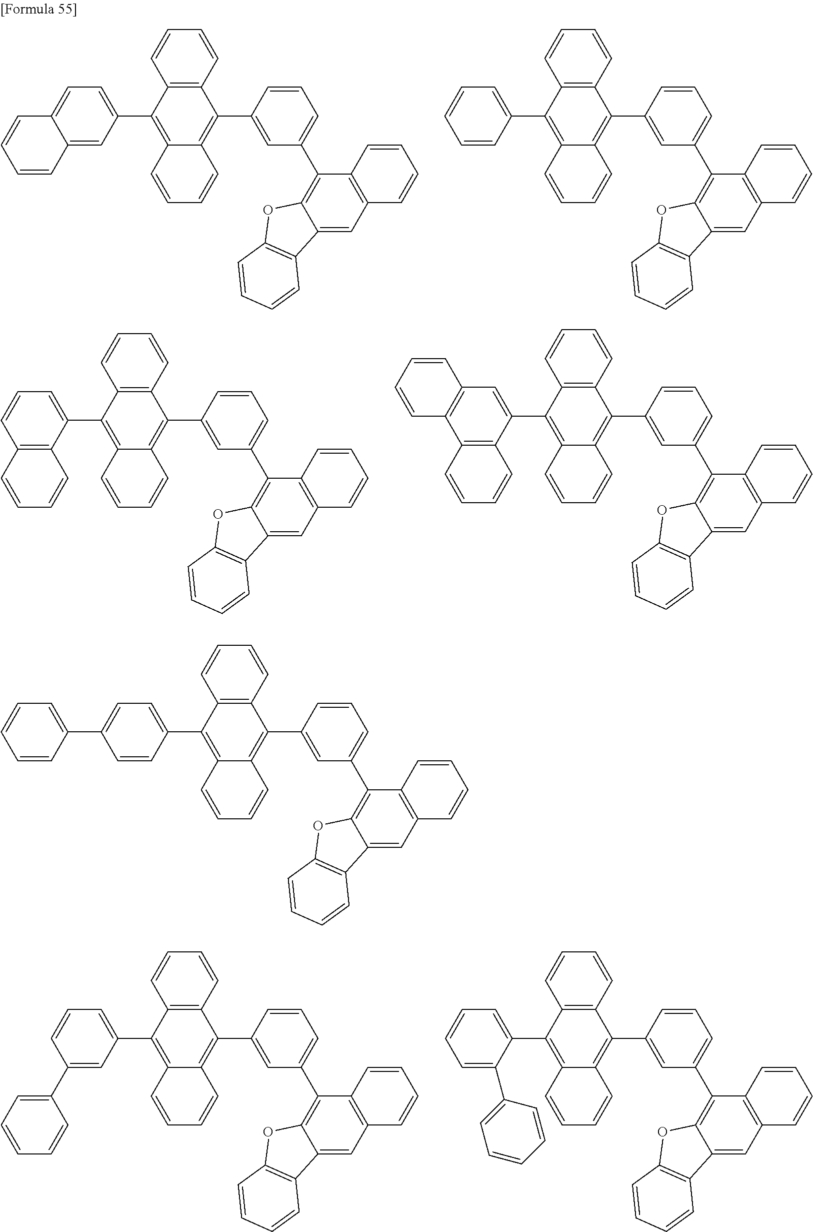

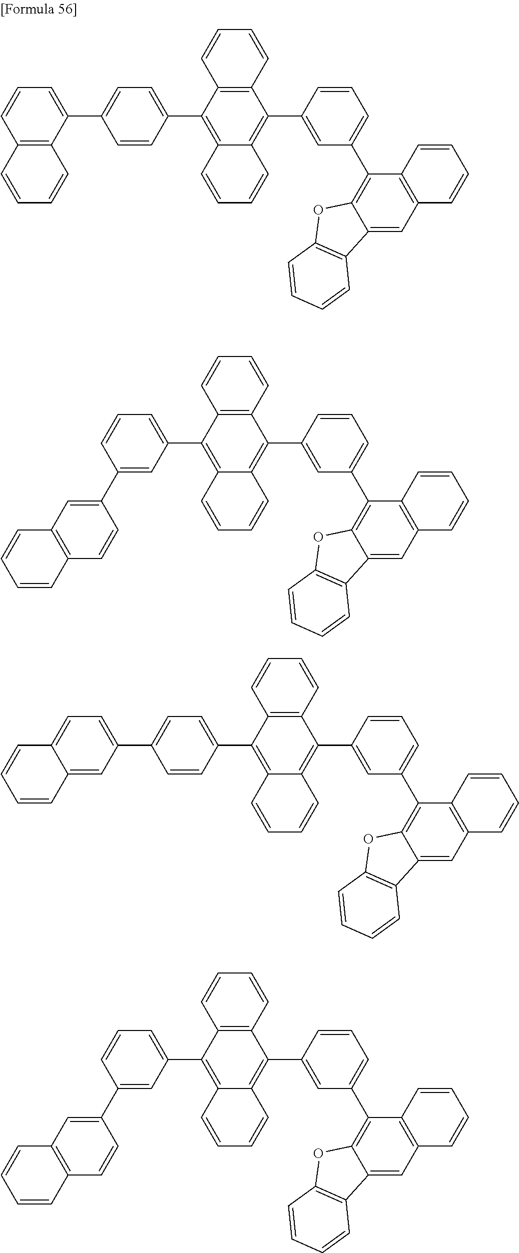

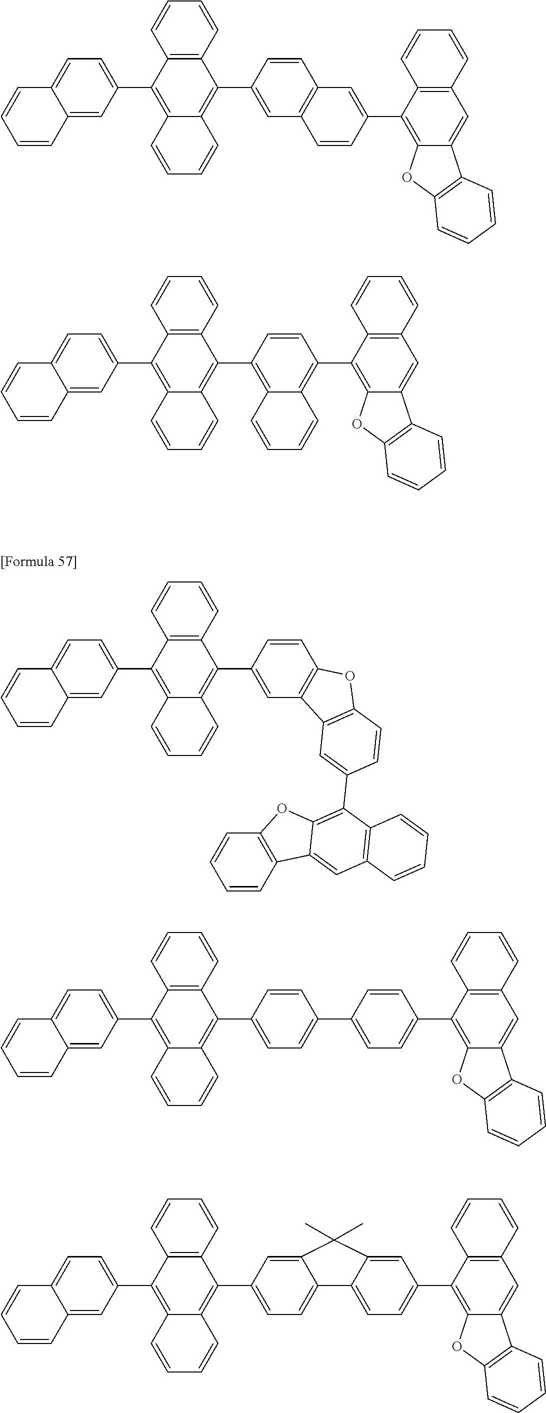 [SMILES: c1ccc2cc(-c3c4ccccc4c(-c4ccc5cc(-c6c7ccccc7cc7c6oc6ccccc67)ccc5c4)c4ccccc34)ccc2c1 ; c1ccc2cc(-c3c4ccccc4c(-c4ccc(-c5c6ccccc6cc6c5oc5ccccc56)c5ccccc45)c4ccccc34)ccc2c1 ; c1ccc2cc(-c3c4ccccc4c(-c4ccc5oc6ccc(-c7c8ccccc8cc8c7oc7ccccc78)cc6c5c4)c4ccccc34)ccc2c1 ; c1ccc2cc(-c3c4ccccc4c(-c4ccc(-c5ccc(-c6c7ccccc7cc7c6oc6ccccc67)cc5)cc4)c4ccccc34)ccc2c1 ; CC1(C)c2cc(-c3c4ccccc4c(-c4ccc5ccccc5c4)c4ccccc34)ccc2-c2ccc(-c3c4ccccc4cc4c3oc3ccccc34)cc21]

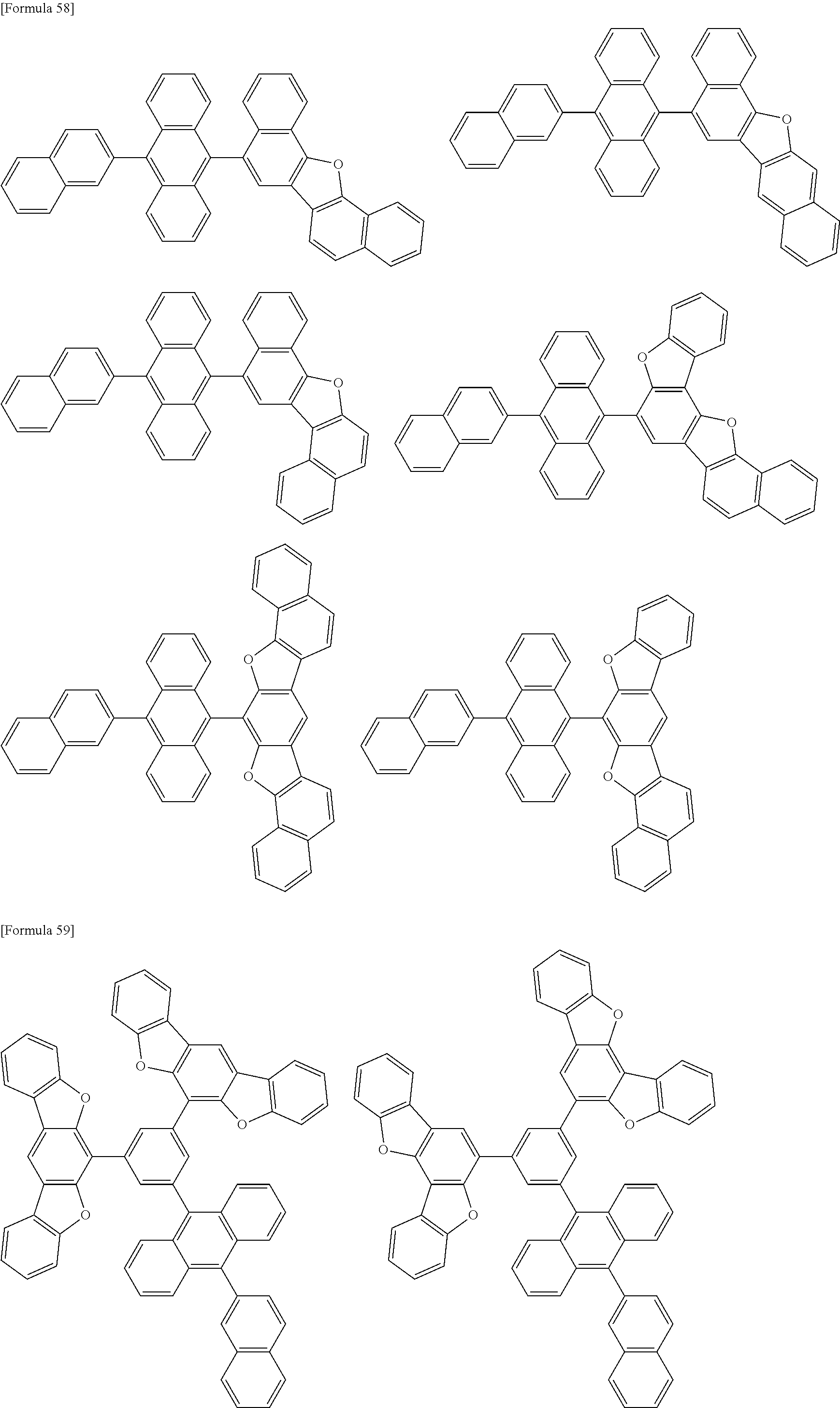

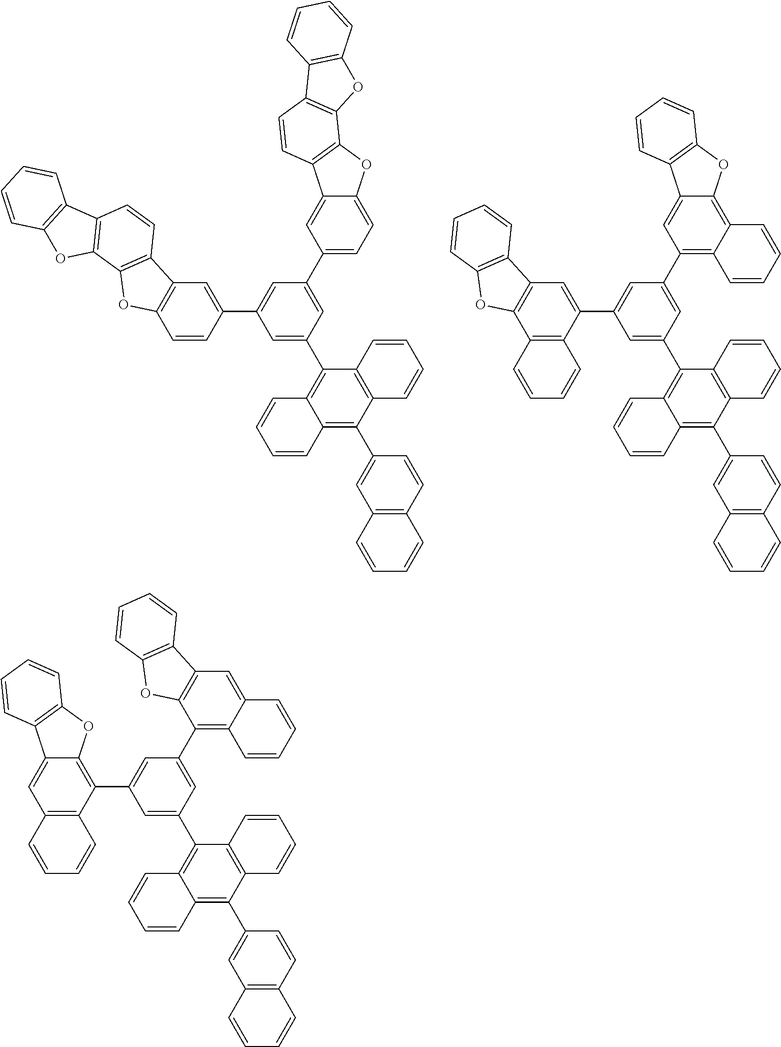 [SMILES: c1ccc2cc(-c3c4ccccc4c(-c4cc(-c5ccc6oc7c(ccc8c9ccccc9oc87)c6c5)cc(-c5ccc6oc7c(ccc8c9ccccc9oc87)c6c5)c4)c4ccccc34)ccc2c1 ; c1ccc2cc(-c3c4ccccc4c(-c4cc(-c5cc6c7ccccc7oc6c6ccccc56)cc(-c5cc6c7ccccc7oc6c6ccccc56)c4)c4ccccc34)ccc2c1 ; c1ccc2cc(-c3c4ccccc4c(-c4cc(-c5c6ccccc6cc6c5oc5ccccc56)cc(-c5c6ccccc6cc6c5oc5ccccc56)c4)c4ccccc34)ccc2c1]

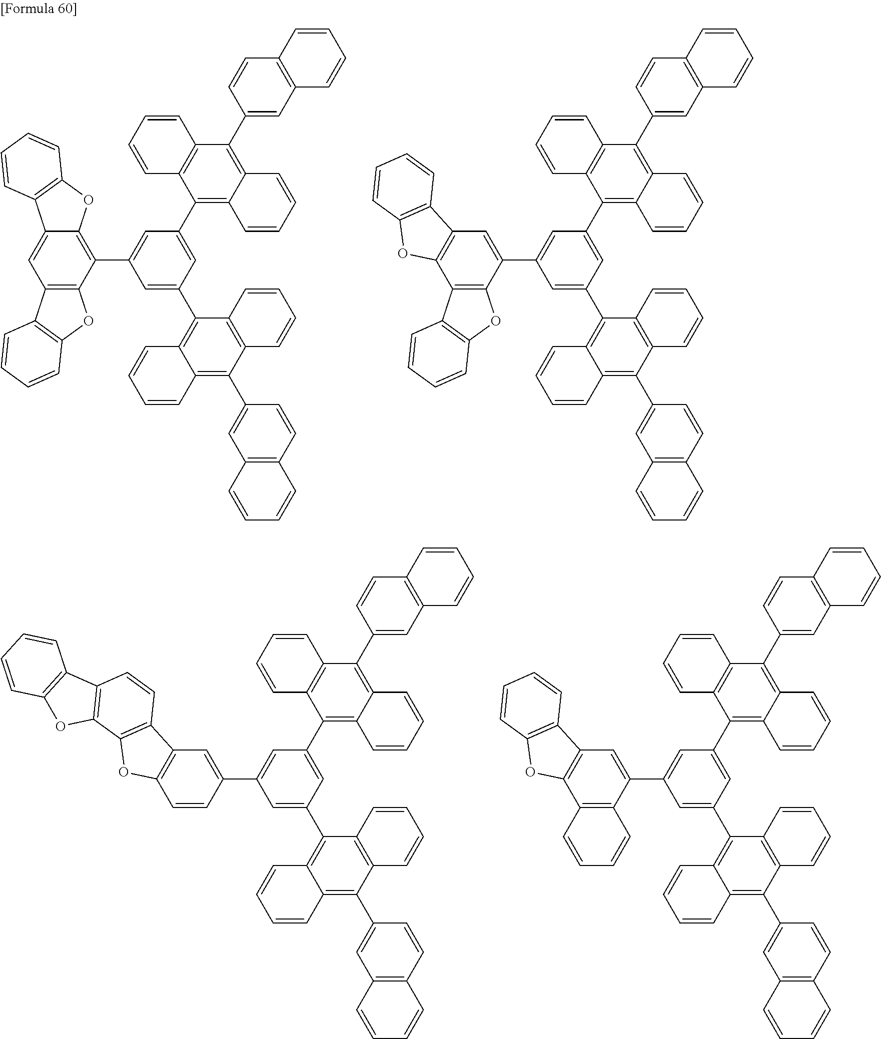 [SMILES: c1ccc2cc(-c3c4ccccc4c(-c4cc(-c5c6ccccc6c(-c6ccc7ccccc7c6)c6ccccc56)cc(-c5c6oc7ccccc7c6cc6c5oc5ccccc56)c4)c4ccccc34)ccc2c1 ; c1ccc2cc(-c3c4ccccc4c(-c4cc(-c5c6ccccc6c(-c6ccc7ccccc7c6)c6ccccc56)cc(-c5cc6c7ccccc7oc6c6c5oc5ccccc56)c4)c4ccccc34)ccc2c1 ; c1ccc2cc(-c3c4ccccc4c(-c4cc(-c5ccc6oc7c(ccc8c9ccccc9oc87)c6c5)cc(-c5c6ccccc6c(-c6ccc7ccccc7c6)c6ccccc56)c4)c4ccccc34)ccc2c1 ; c1ccc2cc(-c3c4ccccc4c(-c4cc(-c5c6ccccc6c(-c6ccc7ccccc7c6)c6ccccc56)cc(-c5cc6c7ccccc7oc6c6ccccc56)c4)c4ccccc34)ccc2c1]

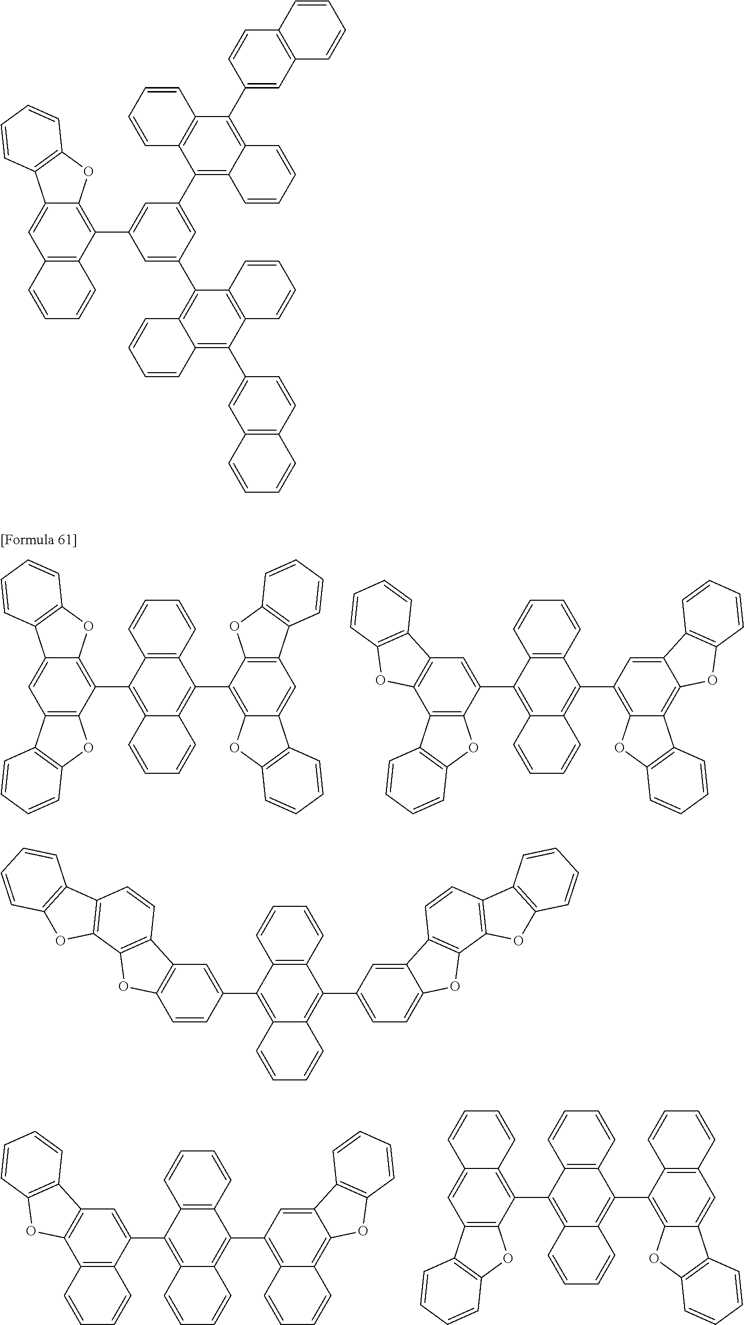

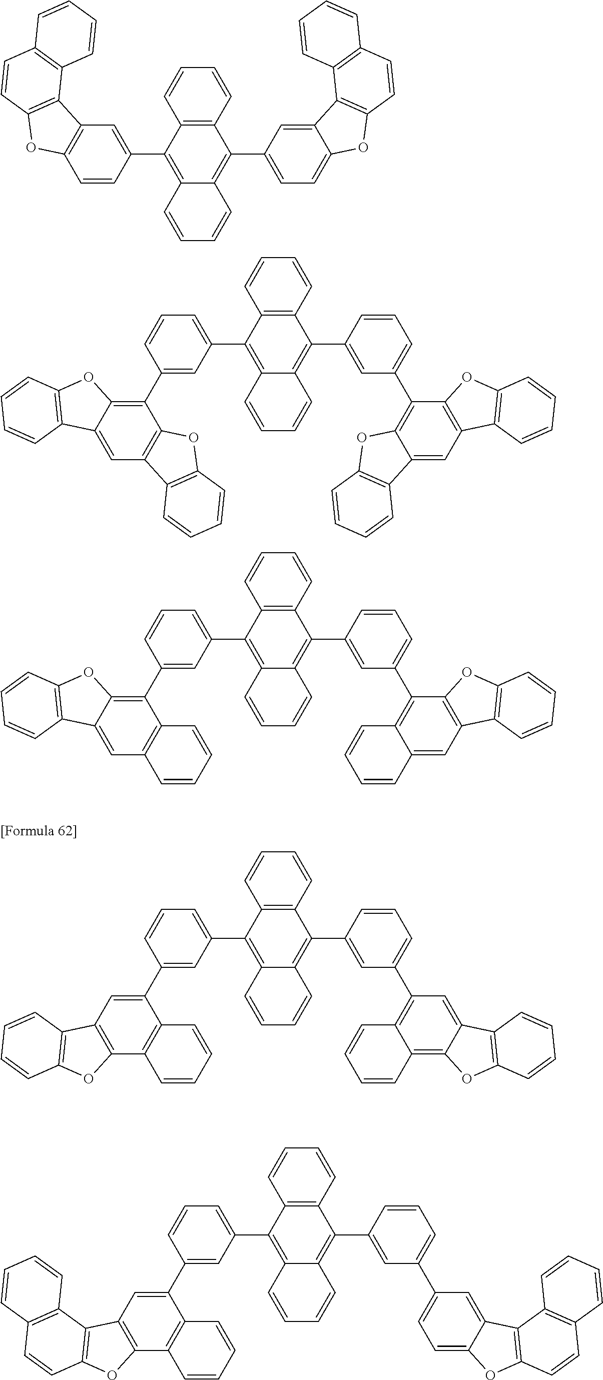 [SMILES: c1ccc2c(c1)ccc1oc3ccc(-c4c5ccccc5c(-c5ccc6oc7ccc8ccccc8c7c6c5)c5ccccc45)cc3c12 ; c1cc(-c2c3ccccc3c(-c3cccc(-c4c5oc6ccccc6c5cc5c4oc4ccccc45)c3)c3ccccc23)cc(-c2c3oc4ccccc4c3cc3c2oc2ccccc23)c1 ; c1cc(-c2c3ccccc3c(-c3cccc(-c4c5ccccc5cc5c4oc4ccccc45)c3)c3ccccc23)cc(-c2c3ccccc3cc3c2oc2ccccc23)c1 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4cc5c(oc6ccc7ccccc7c65)c5ccccc45)c3)c3ccccc23)c1]

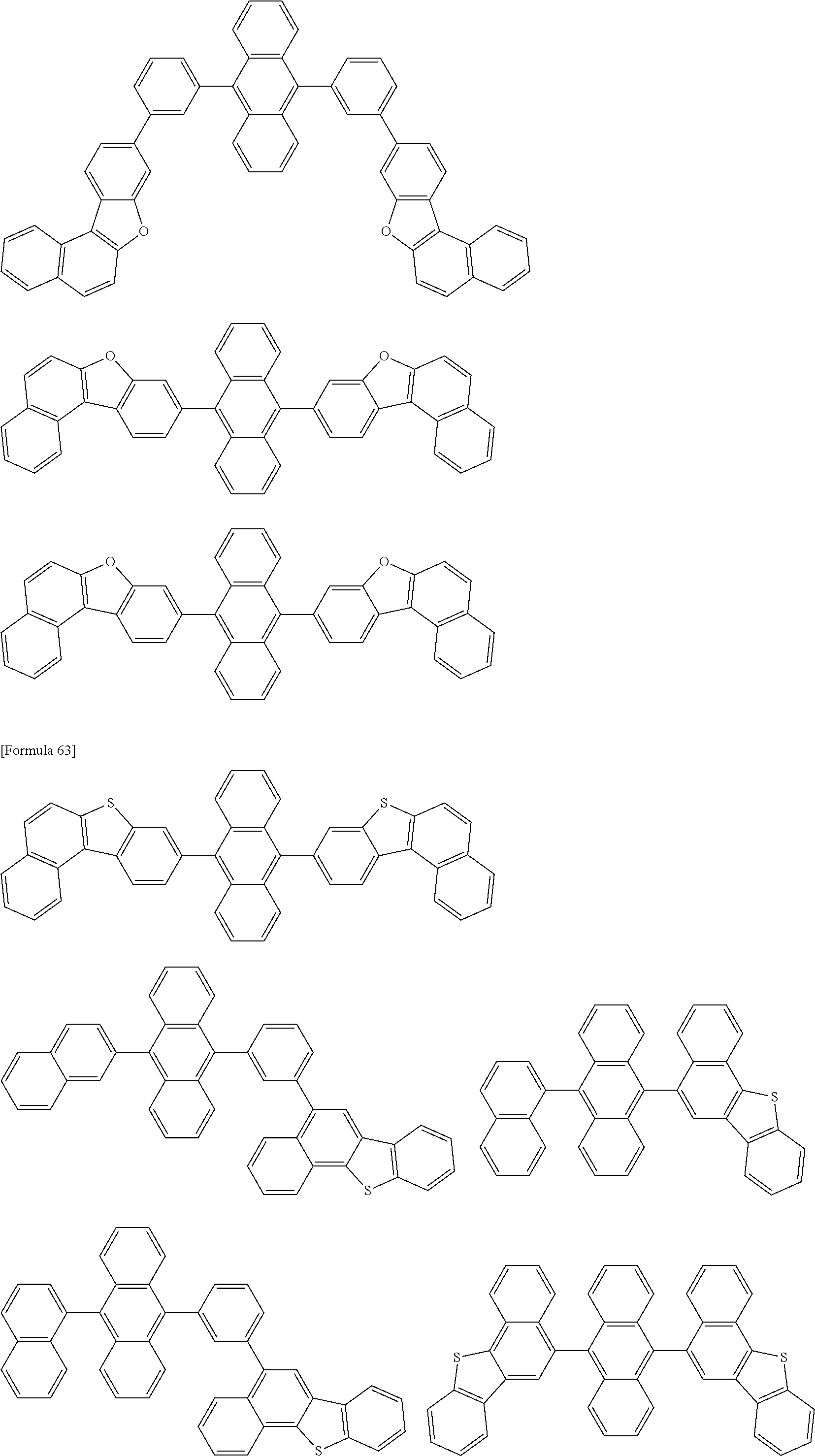

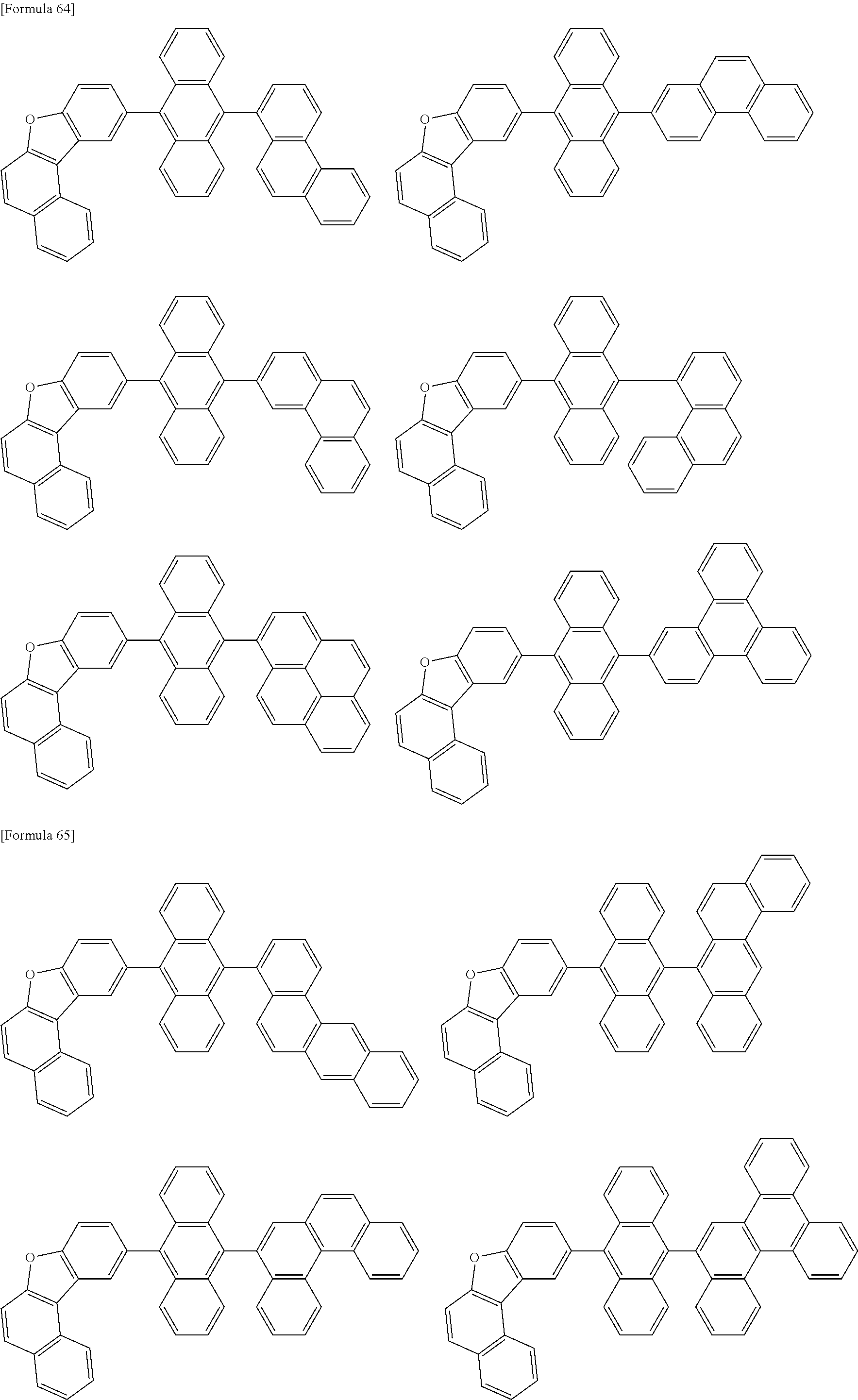

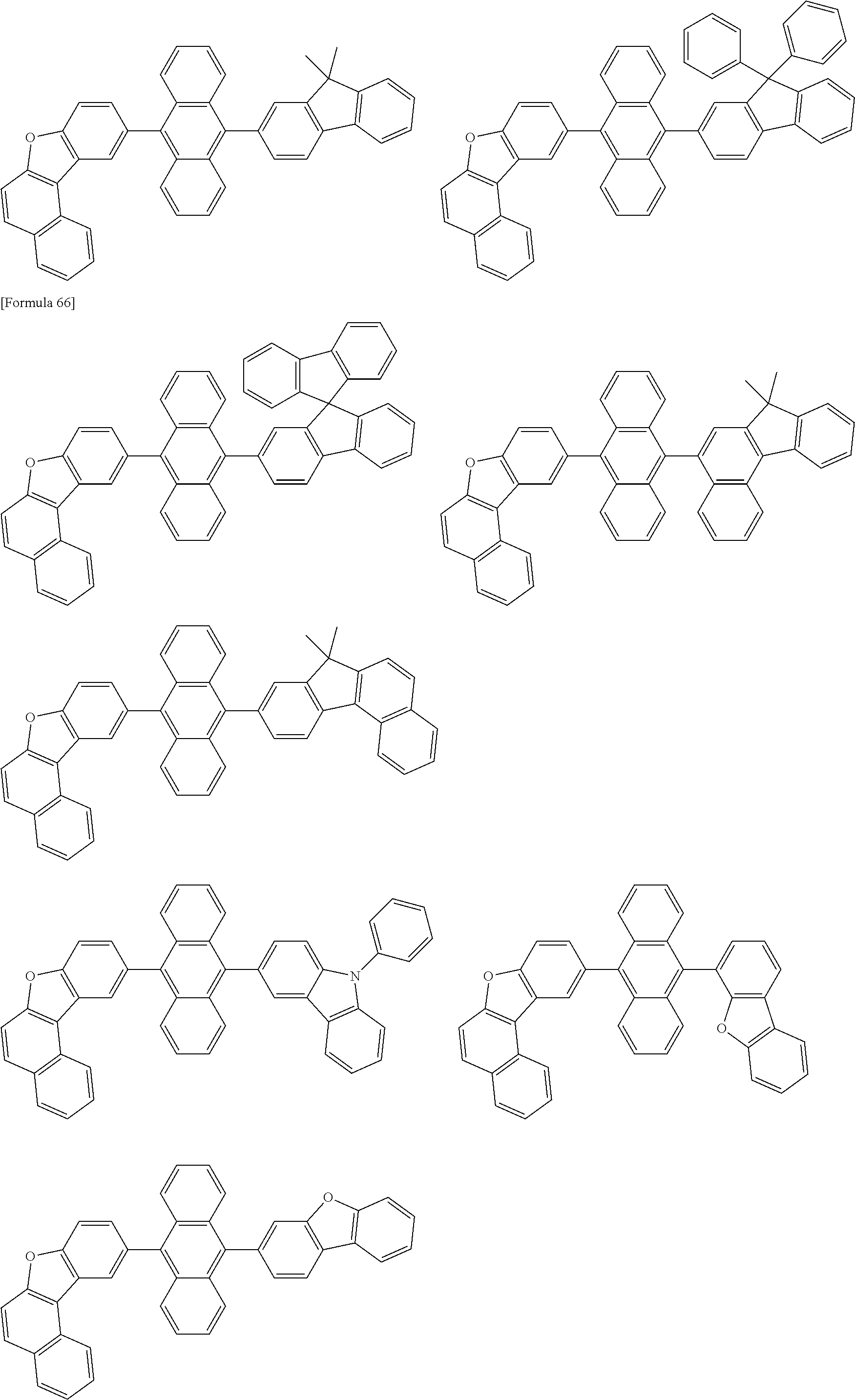

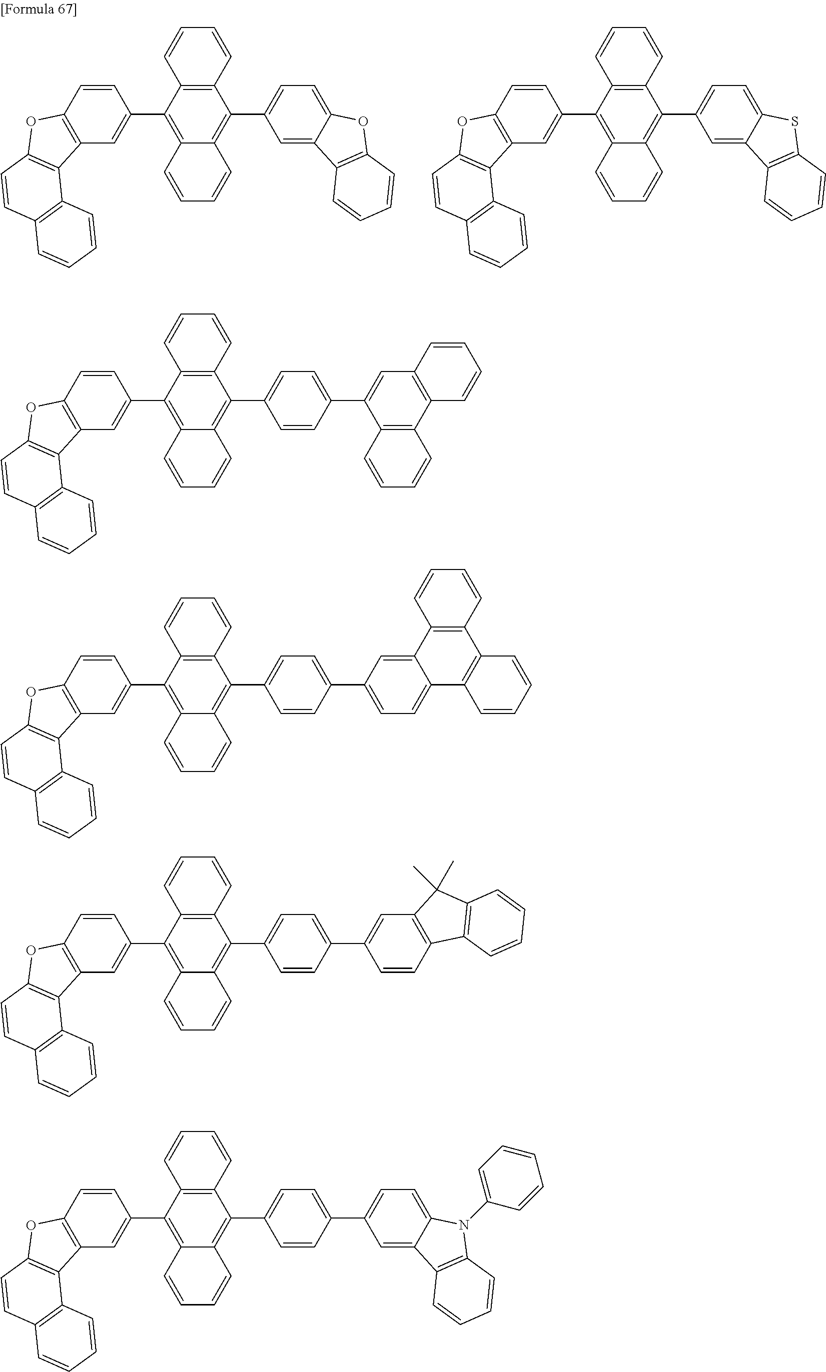

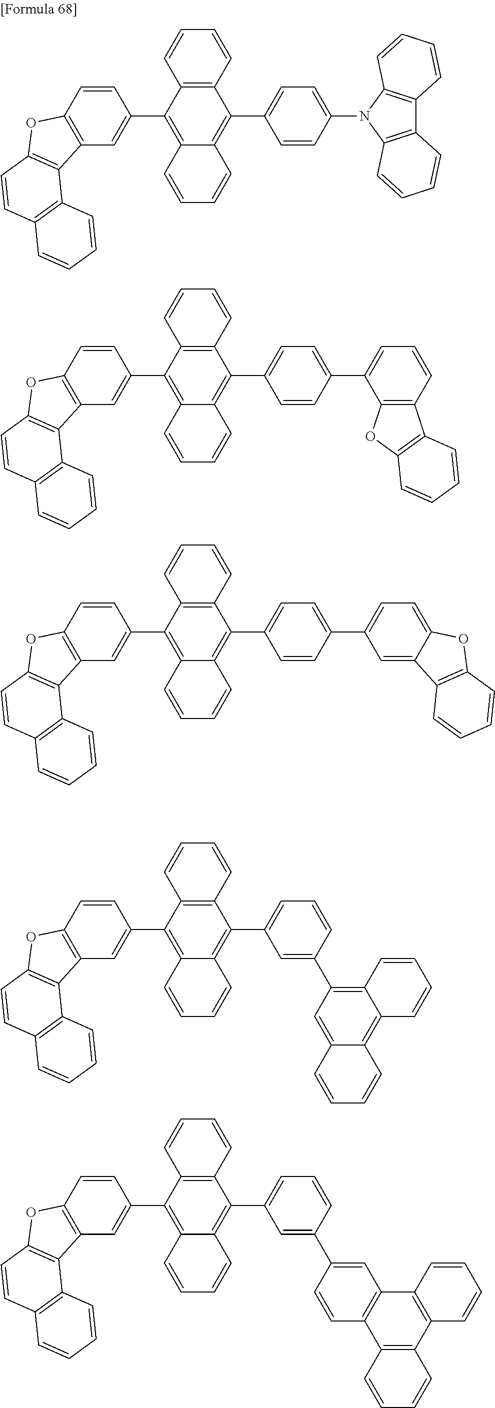 [SMILES: c1ccc2c(c1)ccc1oc3ccc(-c4c5ccccc5c(-c5ccc(-n6c7ccccc7c7ccccc76)cc5)c5ccccc45)cc3c12 ; c1ccc2c(c1)ccc1oc3ccc(-c4c5ccccc5c(-c5ccc(-c6cccc7c6oc6ccccc67)cc5)c5ccccc45)cc3c12 ; c1ccc2c(c1)ccc1oc3ccc(-c4c5ccccc5c(-c5ccc(-c6ccc7oc8ccccc8c7c6)cc5)c5ccccc45)cc3c12 ; c1cc(-c2c3ccccc3c(-c3ccc4oc5ccc6ccccc6c5c4c3)c3ccccc23)cc(-c2cc3ccccc3c3ccccc23)c1 ; c1cc(-c2ccc3c4ccccc4c4ccccc4c3c2)cc(-c2c3ccccc3c(-c3ccc4oc5ccc6ccccc6c5c4c3)c3ccccc23)c1]

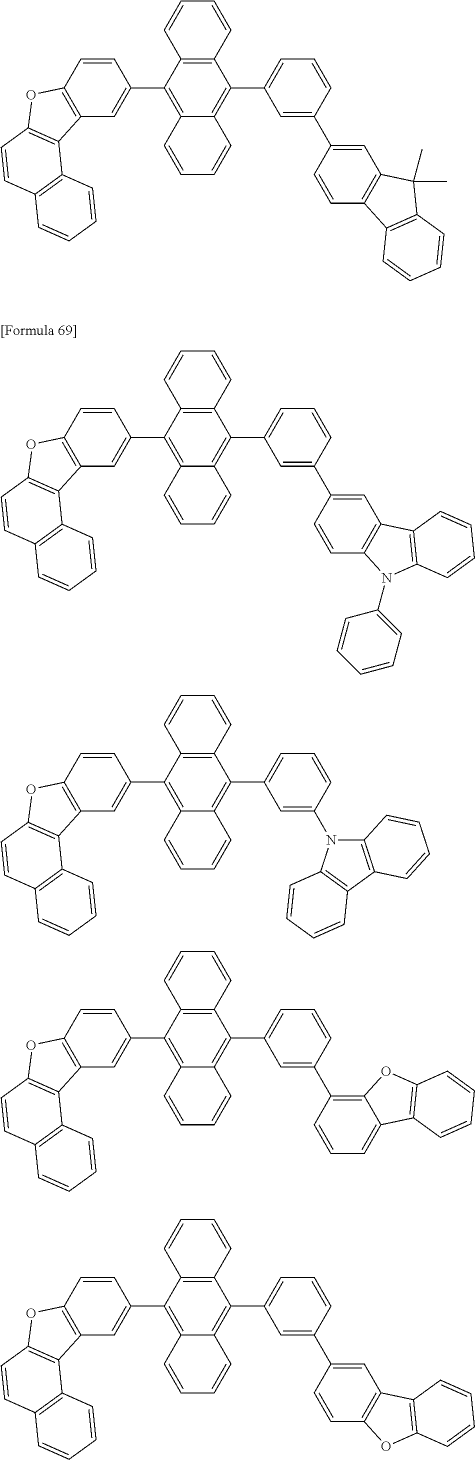 [SMILES: CC1(C)c2ccccc2-c2ccc(-c3cccc(-c4c5ccccc5c(-c5ccc6oc7ccc8ccccc8c7c6c5)c5ccccc45)c3)cc21 ; c1ccc(-n2c3ccccc3c3cc(-c4cccc(-c5c6ccccc6c(-c6ccc7oc8ccc9ccccc9c8c7c6)c6ccccc56)c4)ccc32)cc1 ; c1cc(-c2c3ccccc3c(-c3ccc4oc5ccc6ccccc6c5c4c3)c3ccccc23)cc(-n2c3ccccc3c3ccccc32)c1 ; c1cc(-c2c3ccccc3c(-c3ccc4oc5ccc6ccccc6c5c4c3)c3ccccc23)cc(-c2cccc3c2oc2ccccc23)c1 ; c1cc(-c2ccc3oc4ccccc4c3c2)cc(-c2c3ccccc3c(-c3ccc4oc5ccc6ccccc6c5c4c3)c3ccccc23)c1]

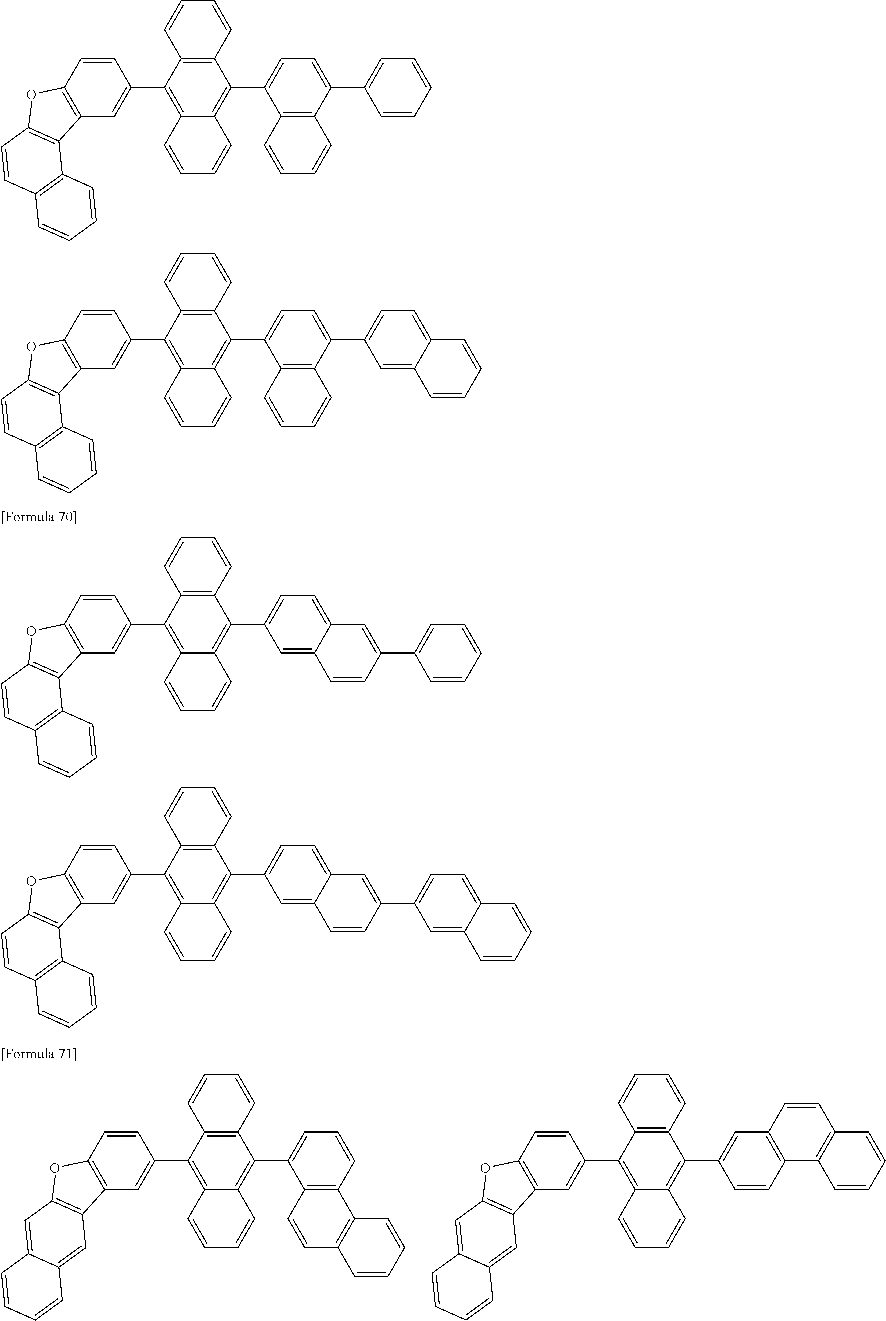

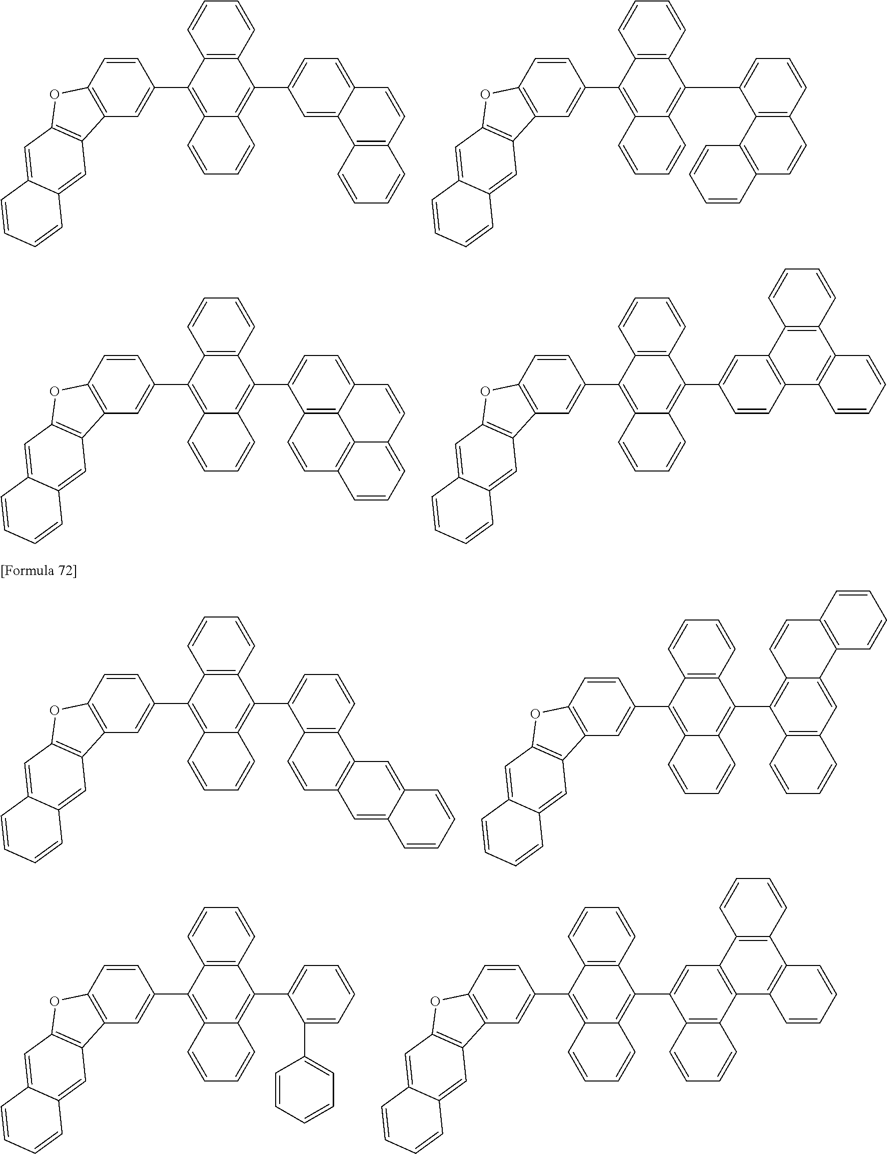

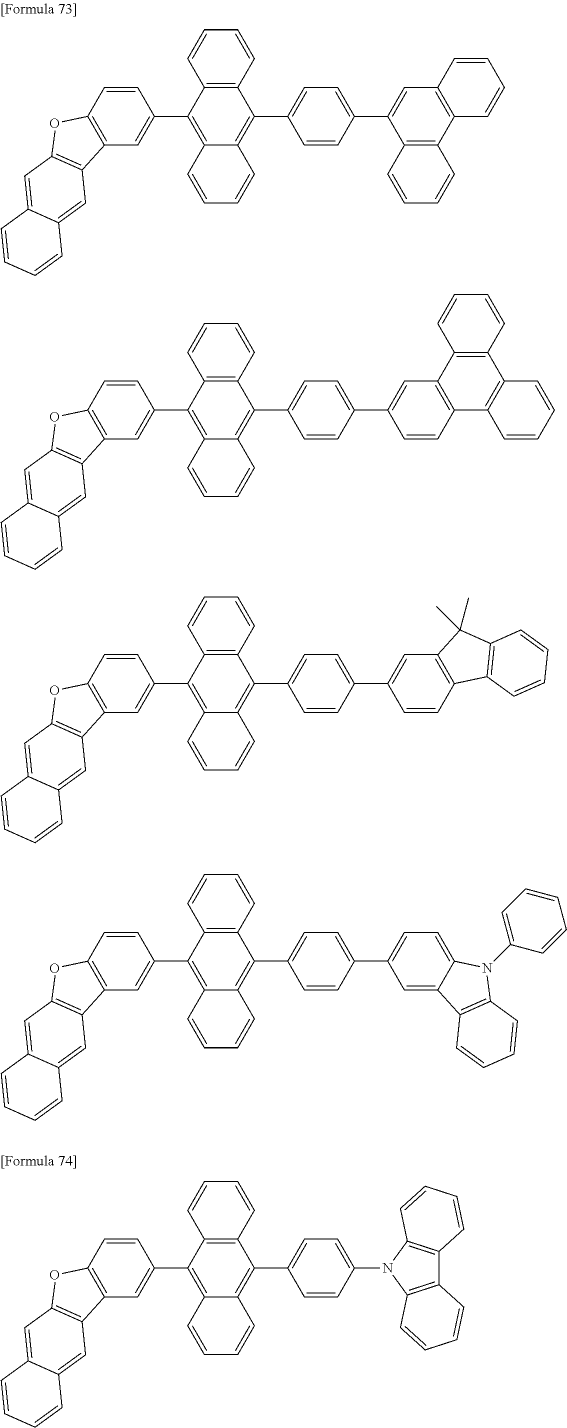 [SMILES: c1ccc2cc3c(cc2c1)oc1ccc(-c2c4ccccc4c(-c4ccc(-c5cc6ccccc6c6ccccc56)cc4)c4ccccc24)cc13 ; c1ccc2cc3c(cc2c1)oc1ccc(-c2c4ccccc4c(-c4ccc(-c5ccc6c7ccccc7c7ccccc7c6c5)cc4)c4ccccc24)cc13 ; CC1(C)c2ccccc2-c2ccc(-c3ccc(-c4c5ccccc5c(-c5ccc6oc7cc8ccccc8cc7c6c5)c5ccccc45)cc3)cc21 ; c1ccc(-n2c3ccccc3c3cc(-c4ccc(-c5c6ccccc6c(-c6ccc7oc8cc9ccccc9cc8c7c6)c6ccccc56)cc4)ccc32)cc1 ; c1ccc2cc3c(cc2c1)oc1ccc(-c2c4ccccc4c(-c4ccc(-n5c6ccccc6c6ccccc65)cc4)c4ccccc24)cc13]

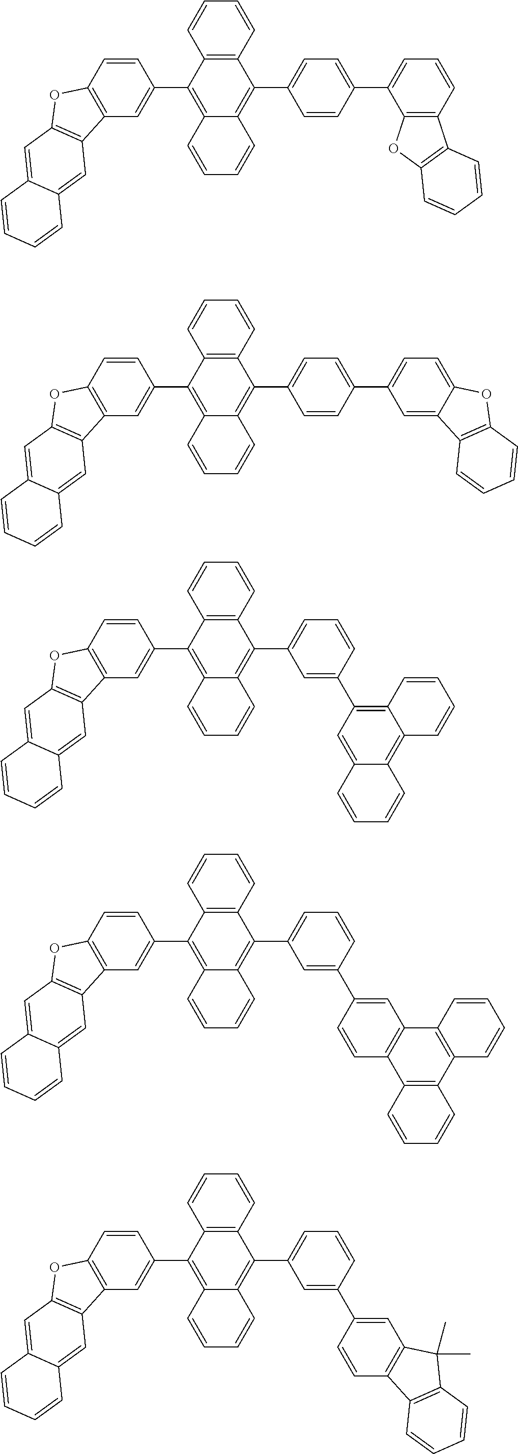 [SMILES: c1ccc2cc3c(cc2c1)oc1ccc(-c2c4ccccc4c(-c4ccc(-c5cccc6c5oc5ccccc56)cc4)c4ccccc24)cc13 ; c1ccc2cc3c(cc2c1)oc1ccc(-c2c4ccccc4c(-c4ccc(-c5ccc6oc7ccccc7c6c5)cc4)c4ccccc24)cc13 ; c1cc(-c2c3ccccc3c(-c3ccc4oc5cc6ccccc6cc5c4c3)c3ccccc23)cc(-c2cc3ccccc3c3ccccc23)c1 ; c1cc(-c2ccc3c4ccccc4c4ccccc4c3c2)cc(-c2c3ccccc3c(-c3ccc4oc5cc6ccccc6cc5c4c3)c3ccccc23)c1 ; CC1(C)c2ccccc2-c2ccc(-c3cccc(-c4c5ccccc5c(-c5ccc6oc7cc8ccccc8cc7c6c5)c5ccccc45)c3)cc21]

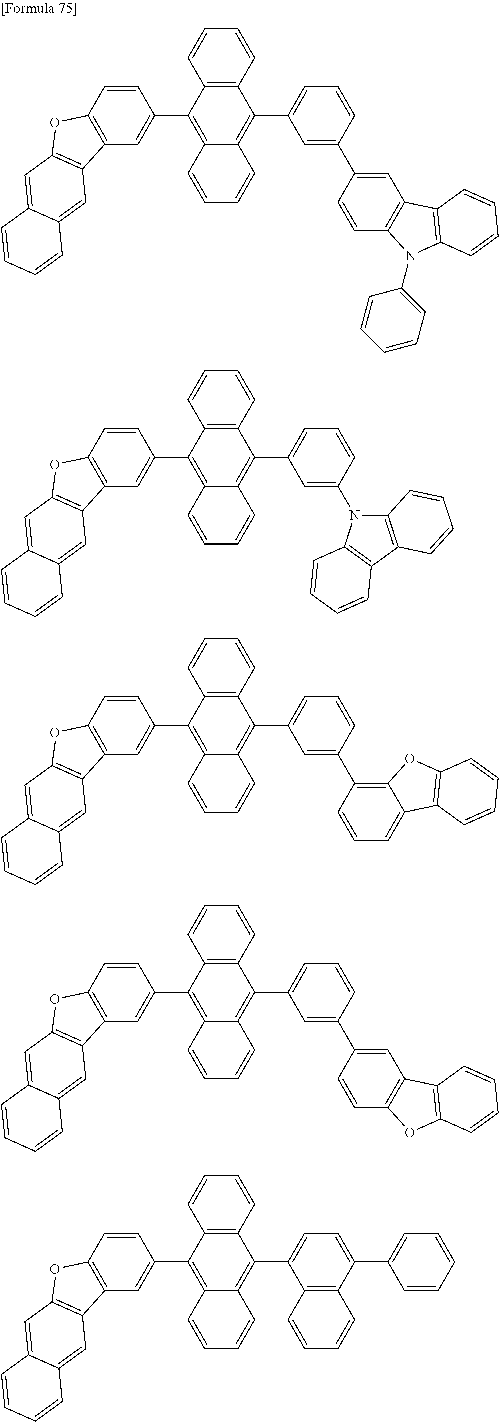 [SMILES: c1ccc(-n2c3ccccc3c3cc(-c4cccc(-c5c6ccccc6c(-c6ccc7oc8cc9ccccc9cc8c7c6)c6ccccc56)c4)ccc32)cc1 ; c1cc(-c2c3ccccc3c(-c3ccc4oc5cc6ccccc6cc5c4c3)c3ccccc23)cc(-n2c3ccccc3c3ccccc32)c1 ; c1cc(-c2c3ccccc3c(-c3ccc4oc5cc6ccccc6cc5c4c3)c3ccccc23)cc(-c2cccc3c2oc2ccccc23)c1 ; c1cc(-c2ccc3oc4ccccc4c3c2)cc(-c2c3ccccc3c(-c3ccc4oc5cc6ccccc6cc5c4c3)c3ccccc23)c1 ; c1ccc(-c2ccc(-c3c4ccccc4c(-c4ccc5oc6cc7ccccc7cc6c5c4)c4ccccc34)c3ccccc23)cc1]

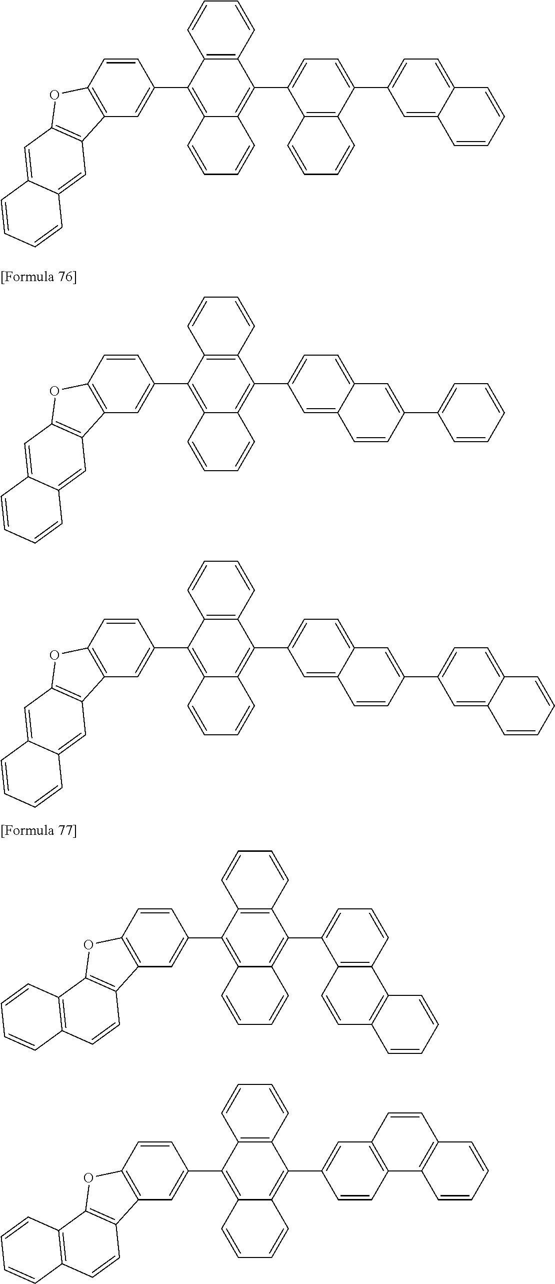 [SMILES: c1ccc2cc(-c3ccc(-c4c5ccccc5c(-c5ccc6oc7cc8ccccc8cc7c6c5)c5ccccc45)c4ccccc34)ccc2c1 ; c1ccc(-c2ccc3cc(-c4c5ccccc5c(-c5ccc6oc7cc8ccccc8cc7c6c5)c5ccccc45)ccc3c2)cc1 ; c1ccc2cc(-c3ccc4cc(-c5c6ccccc6c(-c6ccc7oc8cc9ccccc9cc8c7c6)c6ccccc56)ccc4c3)ccc2c1 ; c1ccc2c(c1)ccc1c(-c3c4ccccc4c(-c4ccc5oc6c7ccccc7ccc6c5c4)c4ccccc34)cccc12 ; c1ccc2c(c1)ccc1cc(-c3c4ccccc4c(-c4ccc5oc6c7ccccc7ccc6c5c4)c4ccccc34)ccc12]

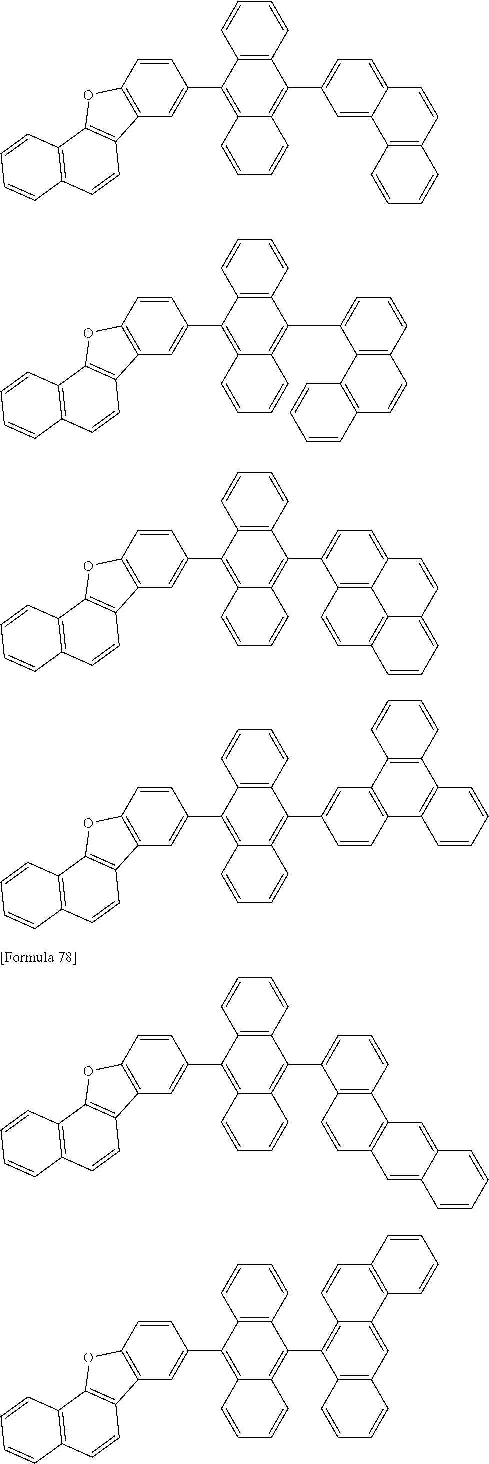

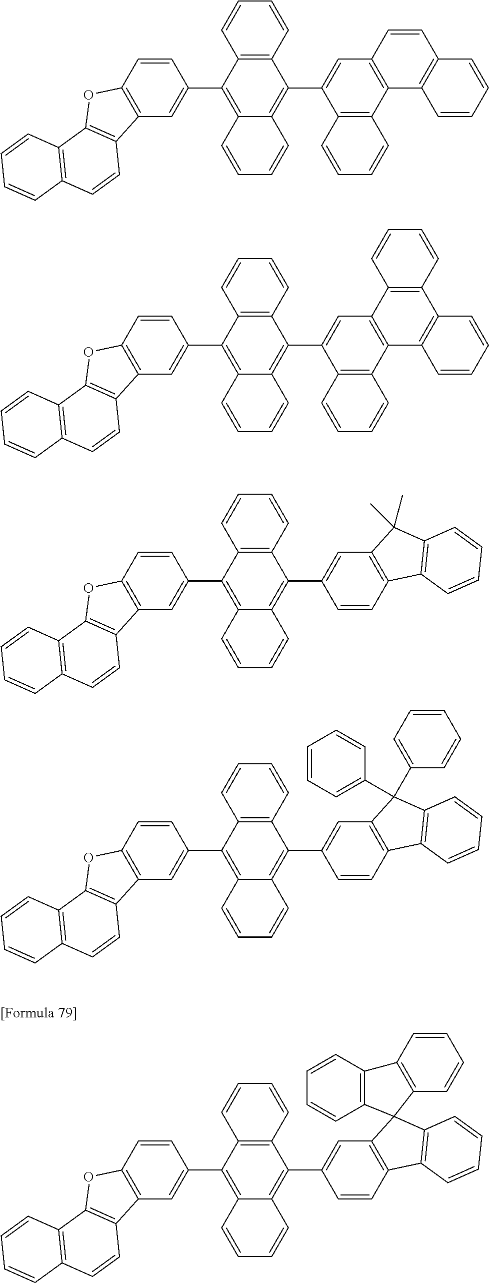 [SMILES: c1ccc2c(c1)ccc1c3cc(-c4c5ccccc5c(-c5cc6ccc7ccccc7c6c6ccccc56)c5ccccc45)ccc3oc21 ; c1ccc2c(c1)ccc1c3cc(-c4c5ccccc5c(-c5cc6c7ccccc7c7ccccc7c6c6ccccc56)c5ccccc45)ccc3oc21 ; CC1(C)c2ccccc2-c2ccc(-c3c4ccccc4c(-c4ccc5oc6c7ccccc7ccc6c5c4)c4ccccc34)cc21 ; c1ccc(C2(c3ccccc3)c3ccccc3-c3ccc(-c4c5ccccc5c(-c5ccc6oc7c8ccccc8ccc7c6c5)c5ccccc45)cc32)cc1 ; c1ccc2c(c1)-c1ccccc1C21c2ccccc2-c2ccc(-c3c4ccccc4c(-c4ccc5oc6c7ccccc7ccc6c5c4)c4ccccc34)cc21]

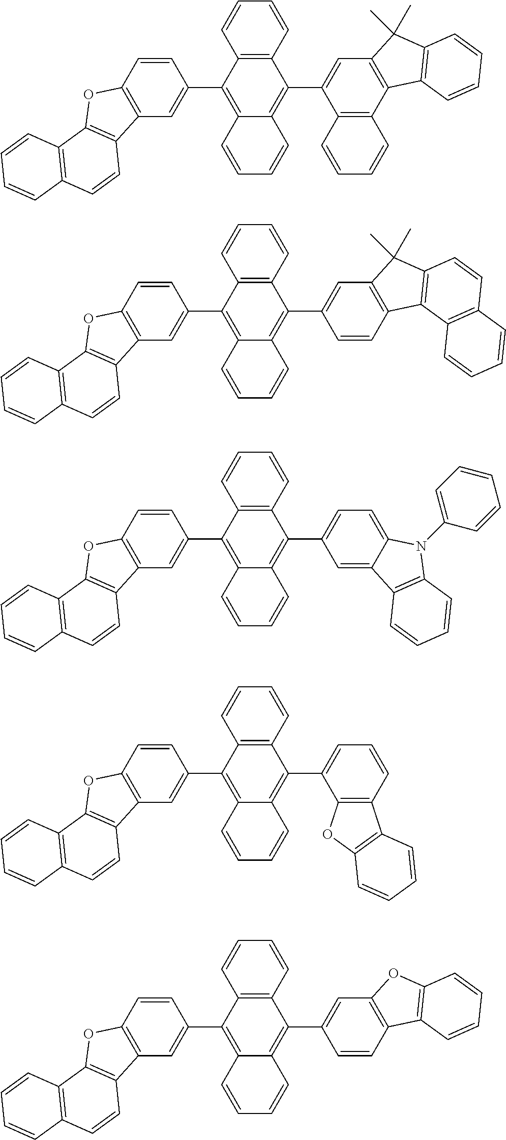 [SMILES: CC1(C)c2ccccc2-c2c1cc(-c1c3ccccc3c(-c3ccc4oc5c6ccccc6ccc5c4c3)c3ccccc13)c1ccccc21 ; CC1(C)c2cc(-c3c4ccccc4c(-c4ccc5oc6c7ccccc7ccc6c5c4)c4ccccc34)ccc2-c2c1ccc1ccccc21 ; c1ccc(-n2c3ccccc3c3cc(-c4c5ccccc5c(-c5ccc6oc7c8ccccc8ccc7c6c5)c5ccccc45)ccc32)cc1 ; c1ccc2c(c1)ccc1c3cc(-c4c5ccccc5c(-c5cccc6c5oc5ccccc56)c5ccccc45)ccc3oc21 ; c1ccc2c(c1)ccc1c3cc(-c4c5ccccc5c(-c5ccc6c(c5)oc5ccccc56)c5ccccc45)ccc3oc21]

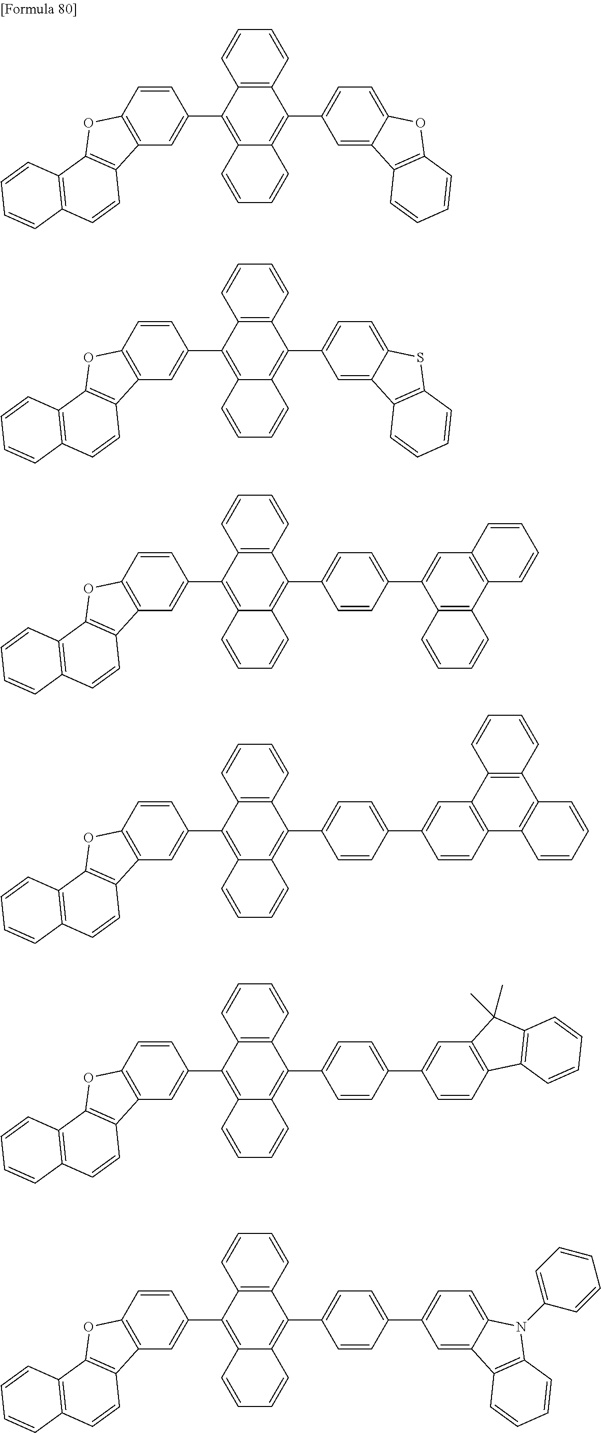

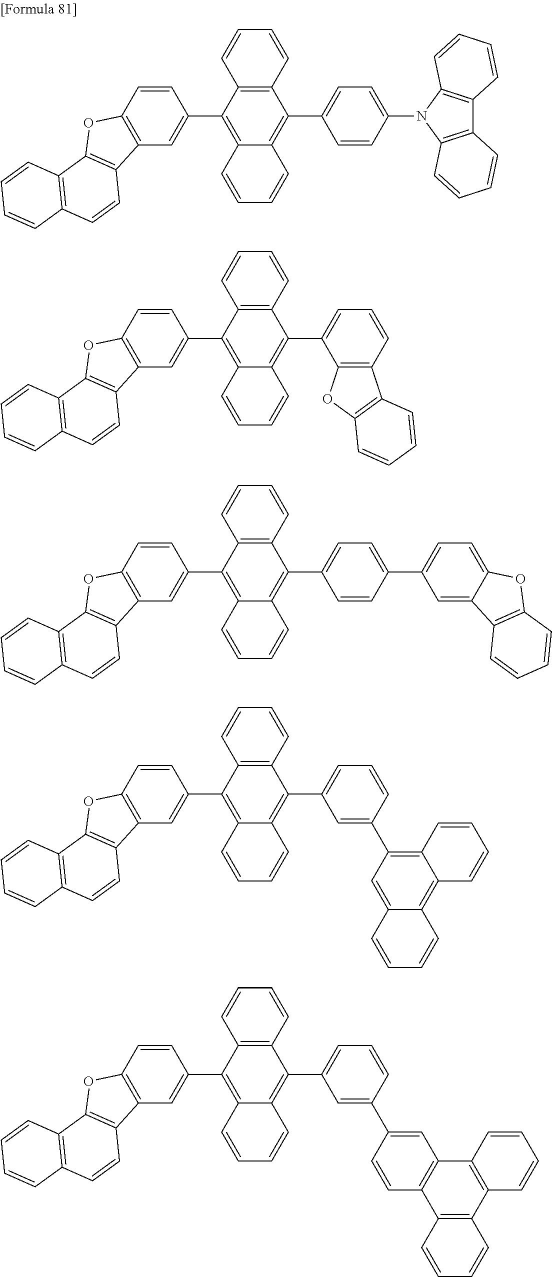 [SMILES: c1ccc2c(c1)ccc1c3cc(-c4c5ccccc5c(-c5ccc(-n6c7ccccc7c7ccccc76)cc5)c5ccccc45)ccc3oc21 ; c1ccc2c(c1)ccc1c3cc(-c4c5ccccc5c(-c5cccc6c5oc5ccccc56)c5ccccc45)ccc3oc21 ; c1ccc2c(c1)ccc1c3cc(-c4c5ccccc5c(-c5ccc(-c6ccc7oc8ccccc8c7c6)cc5)c5ccccc45)ccc3oc21 ; c1cc(-c2c3ccccc3c(-c3ccc4oc5c6ccccc6ccc5c4c3)c3ccccc23)cc(-c2cc3ccccc3c3ccccc23)c1 ; c1cc(-c2ccc3c4ccccc4c4ccccc4c3c2)cc(-c2c3ccccc3c(-c3ccc4oc5c6ccccc6ccc5c4c3)c3ccccc23)c1]

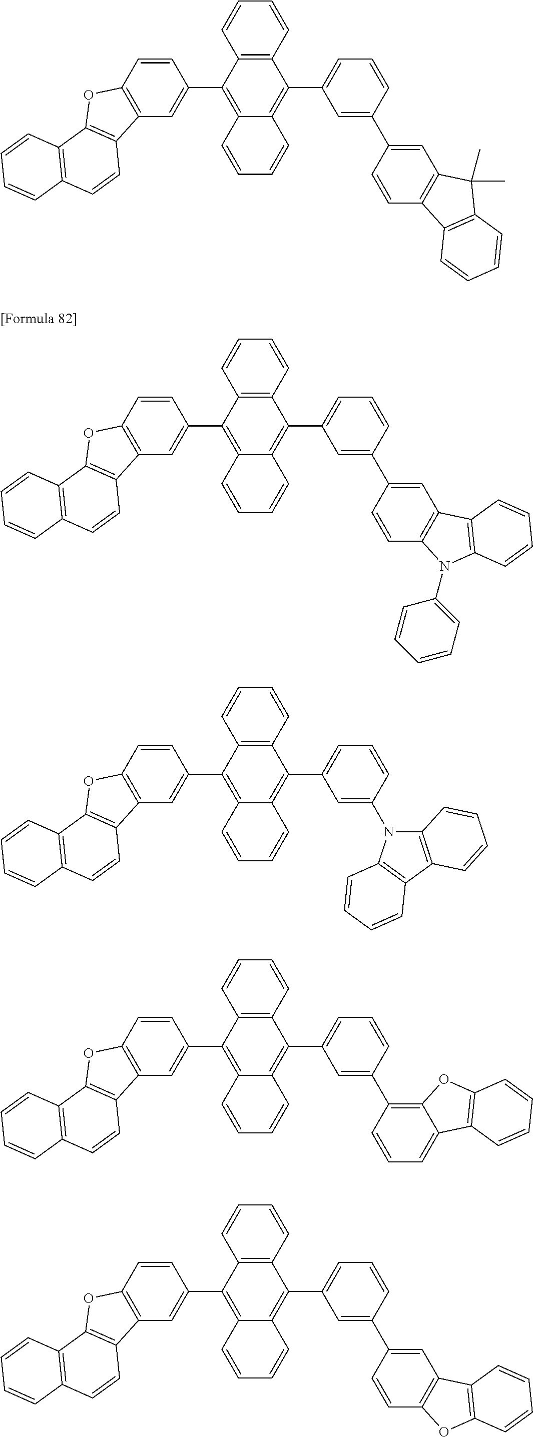 [SMILES: CC1(C)c2ccccc2-c2ccc(-c3cccc(-c4c5ccccc5c(-c5ccc6oc7c8ccccc8ccc7c6c5)c5ccccc45)c3)cc21 ; c1ccc(-n2c3ccccc3c3cc(-c4cccc(-c5c6ccccc6c(-c6ccc7oc8c9ccccc9ccc8c7c6)c6ccccc56)c4)ccc32)cc1 ; c1cc(-c2c3ccccc3c(-c3ccc4oc5c6ccccc6ccc5c4c3)c3ccccc23)cc(-n2c3ccccc3c3ccccc32)c1 ; c1cc(-c2c3ccccc3c(-c3ccc4oc5c6ccccc6ccc5c4c3)c3ccccc23)cc(-c2cccc3c2oc2ccccc23)c1 ; c1cc(-c2ccc3oc4ccccc4c3c2)cc(-c2c3ccccc3c(-c3ccc4oc5c6ccccc6ccc5c4c3)c3ccccc23)c1]

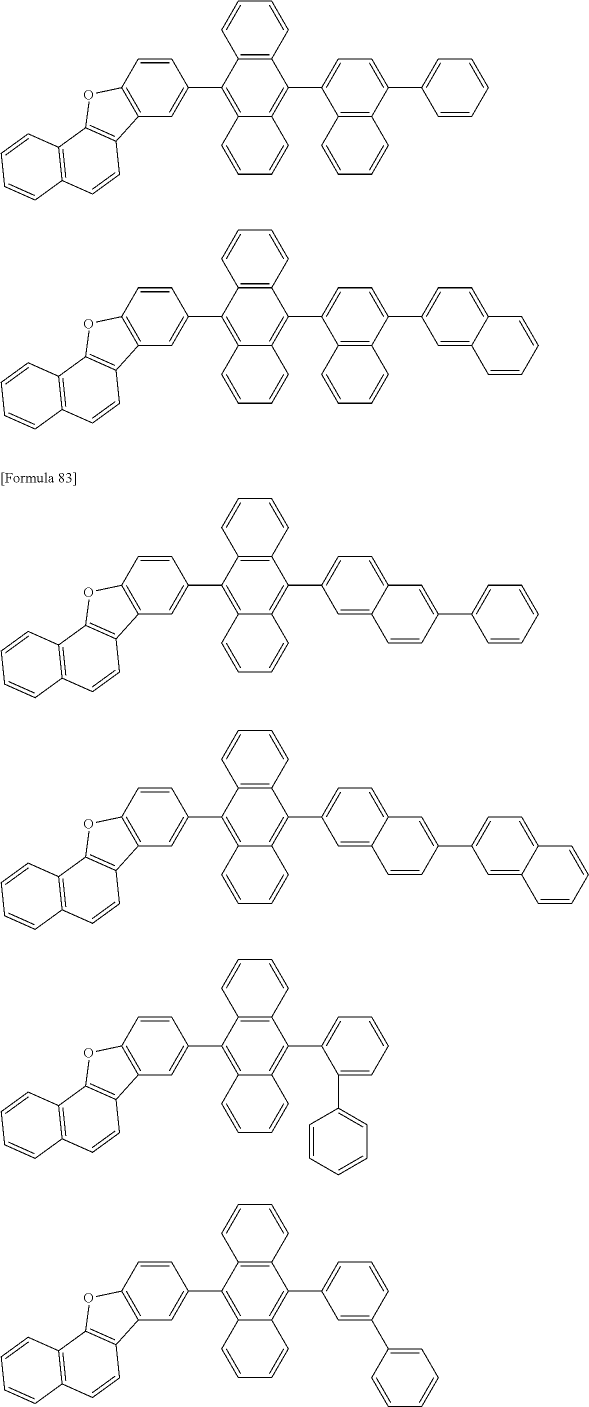 [SMILES: c1ccc(-c2ccc(-c3c4ccccc4c(-c4ccc5oc6c7ccccc7ccc6c5c4)c4ccccc34)c3ccccc23)cc1 ; c1ccc2cc(-c3ccc(-c4c5ccccc5c(-c5ccc6oc7c8ccccc8ccc7c6c5)c5ccccc45)c4ccccc34)ccc2c1 ; c1ccc(-c2ccc3cc(-c4c5ccccc5c(-c5ccc6oc7c8ccccc8ccc7c6c5)c5ccccc45)ccc3c2)cc1 ; c1ccc2cc(-c3ccc4cc(-c5c6ccccc6c(-c6ccc7oc8c9ccccc9ccc8c7c6)c6ccccc56)ccc4c3)ccc2c1 ; c1ccc(-c2ccccc2-c2c3ccccc3c(-c3ccc4oc5c6ccccc6ccc5c4c3)c3ccccc23)cc1 ; c1ccc(-c2cccc(-c3c4ccccc4c(-c4ccc5oc6c7ccccc7ccc6c5c4)c4ccccc34)c2)cc1]

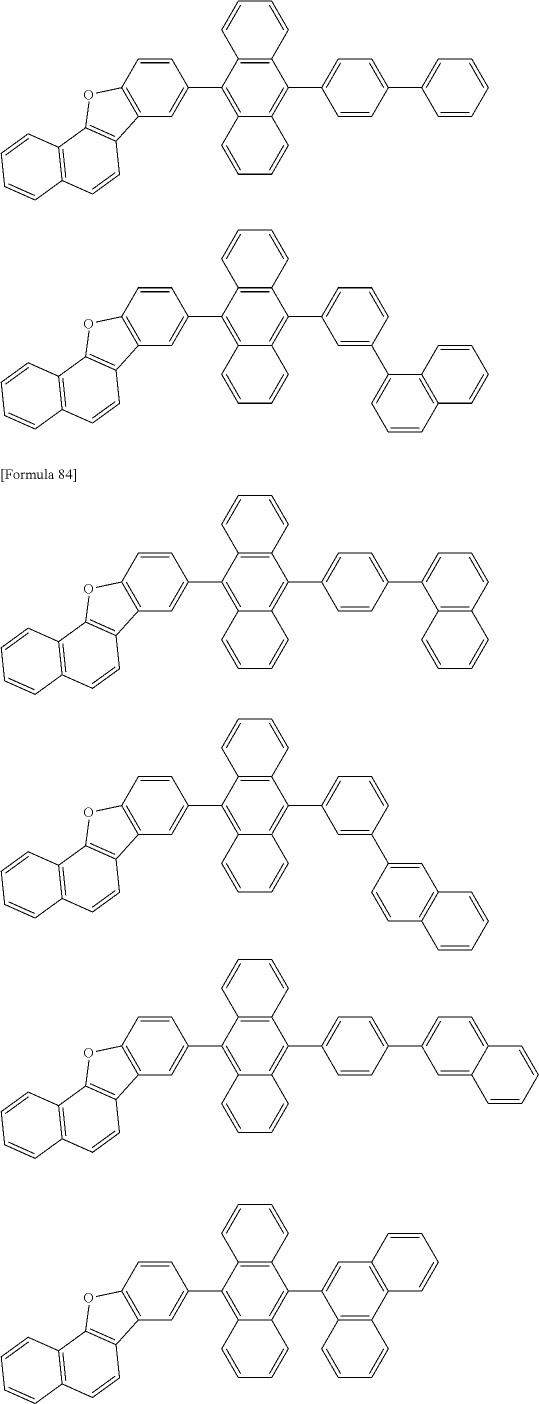 [SMILES: c1ccc(-c2ccc(-c3c4ccccc4c(-c4ccc5oc6c7ccccc7ccc6c5c4)c4ccccc34)cc2)cc1 ; c1cc(-c2cccc3ccccc23)cc(-c2c3ccccc3c(-c3ccc4oc5c6ccccc6ccc5c4c3)c3ccccc23)c1 ; c1ccc2c(-c3ccc(-c4c5ccccc5c(-c5ccc6oc7c8ccccc8ccc7c6c5)c5ccccc45)cc3)cccc2c1 ; c1cc(-c2ccc3ccccc3c2)cc(-c2c3ccccc3c(-c3ccc4oc5c6ccccc6ccc5c4c3)c3ccccc23)c1 ; c1ccc2cc(-c3ccc(-c4c5ccccc5c(-c5ccc6oc7c8ccccc8ccc7c6c5)c5ccccc45)cc3)ccc2c1 ; c1ccc2c(c1)cc(-c1c3ccccc3c(-c3ccc4oc5c6ccccc6ccc5c4c3)c3ccccc13)c1ccccc12]

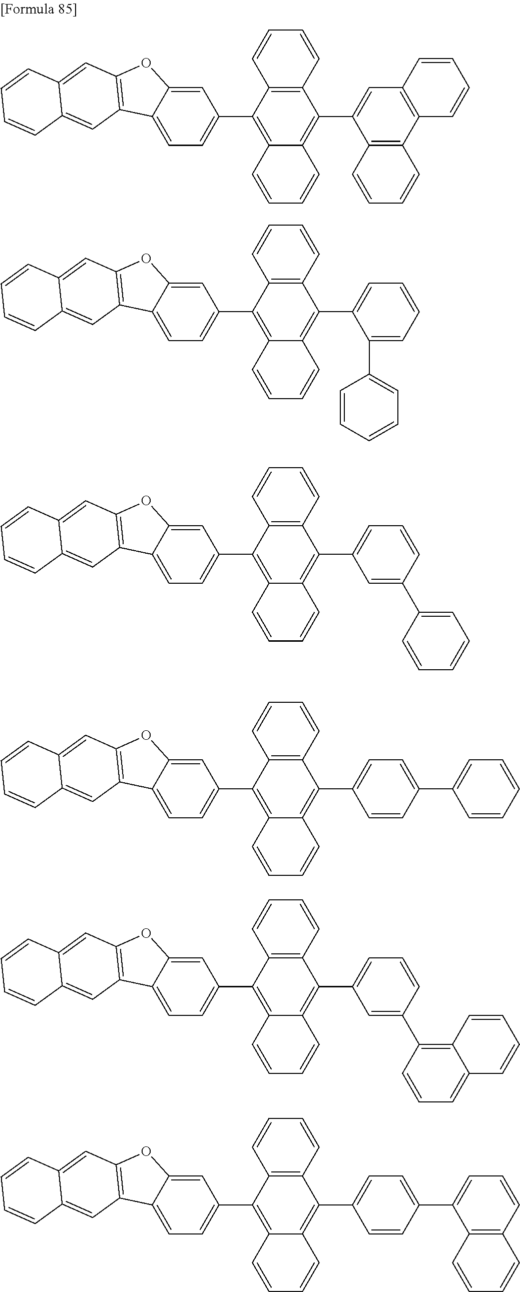

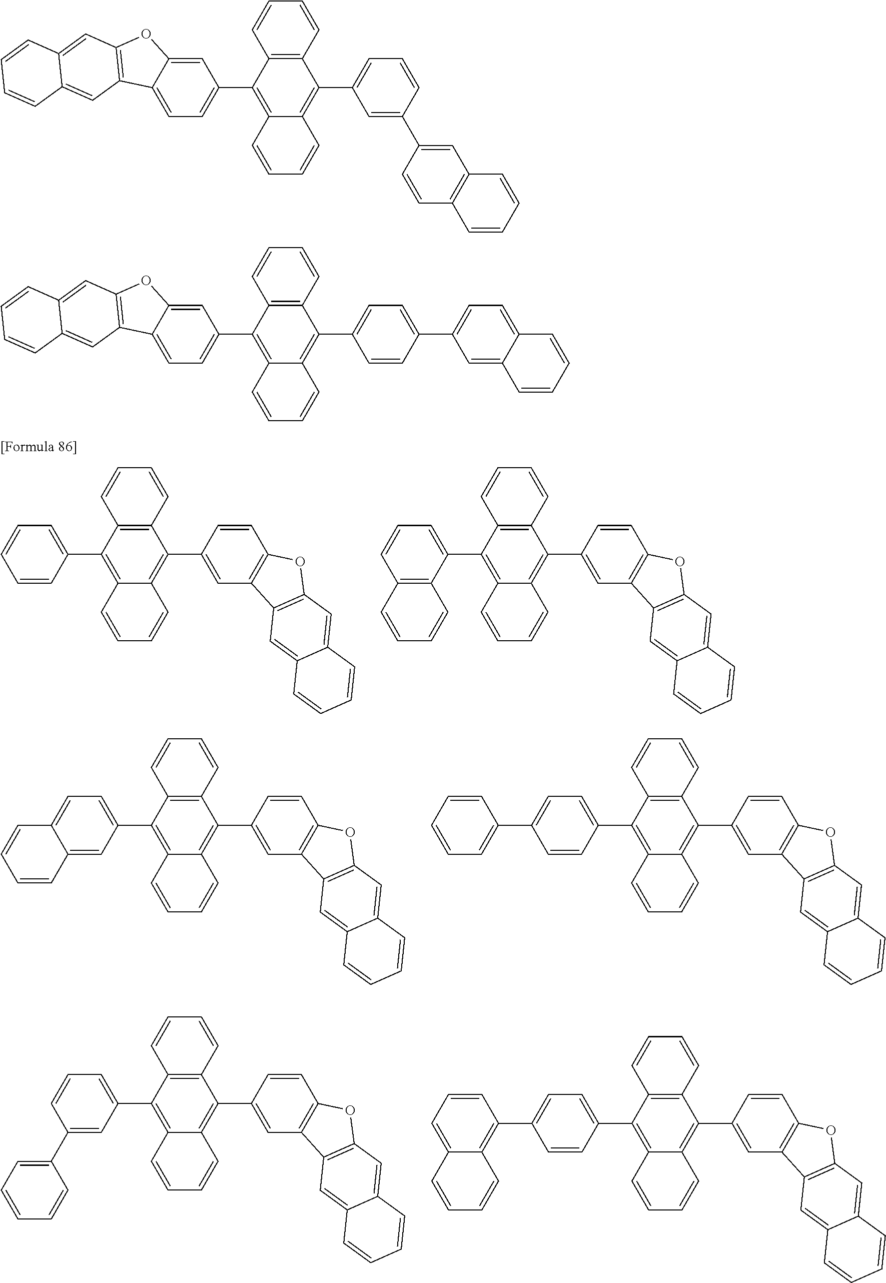

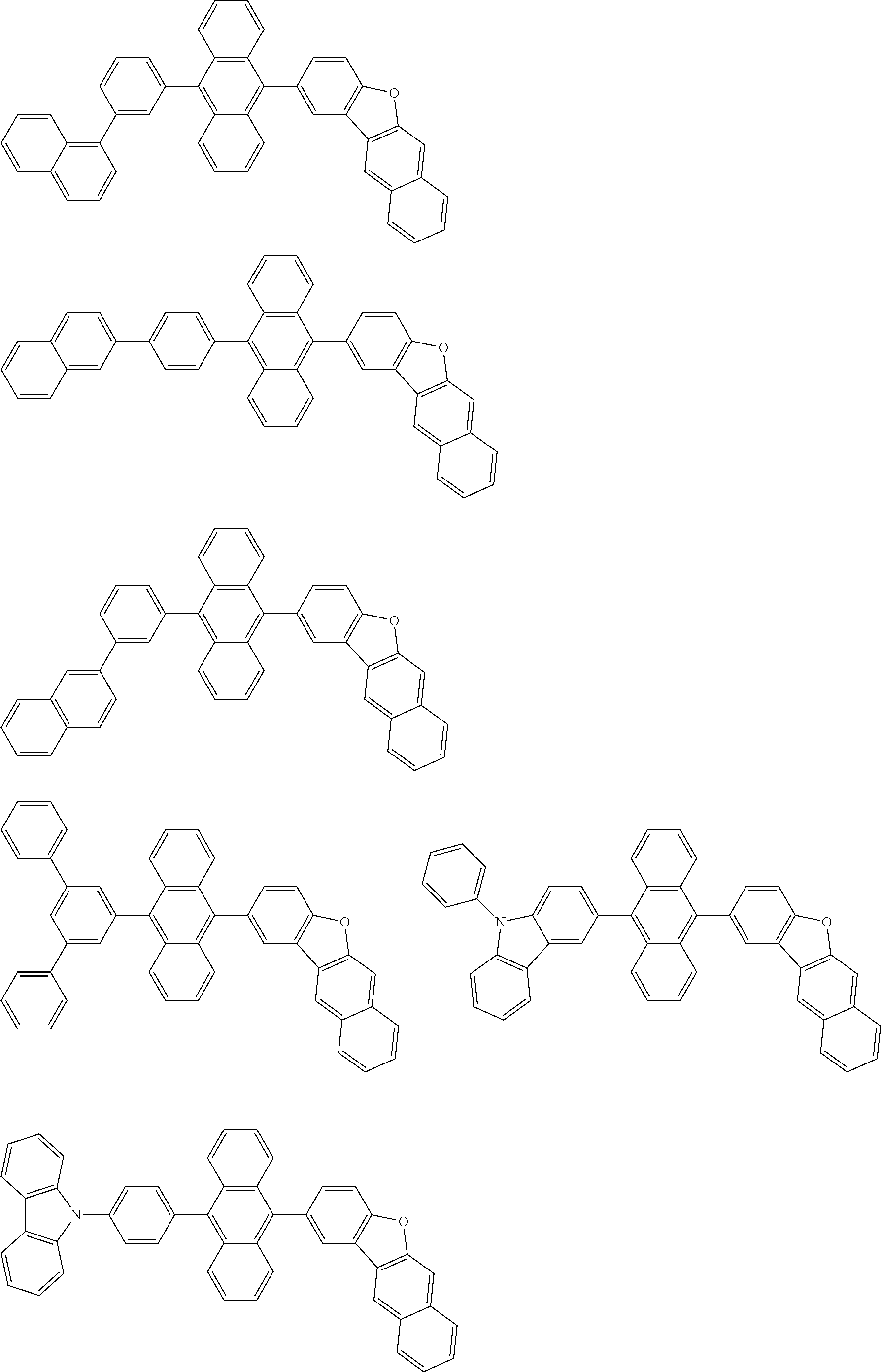

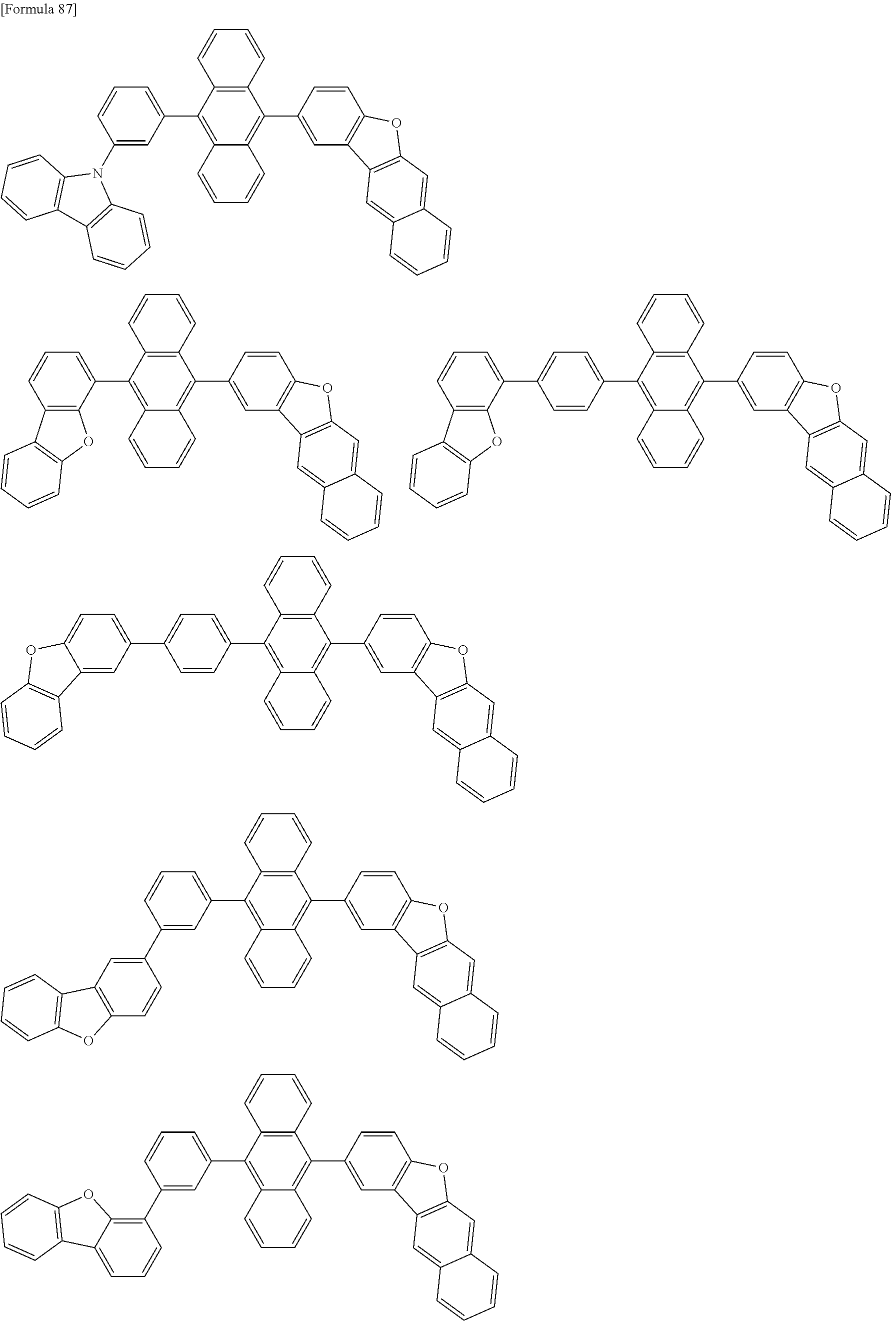

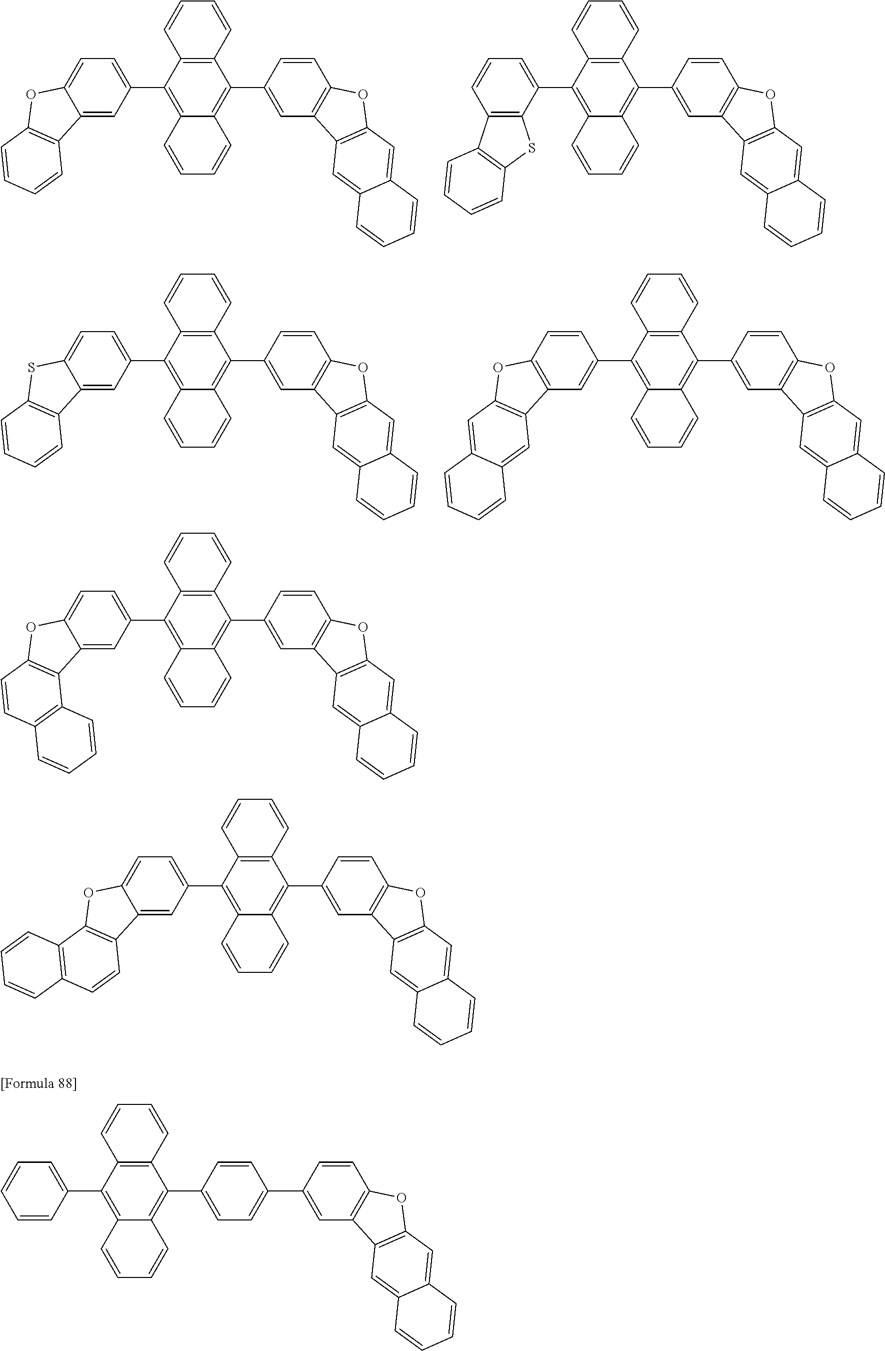

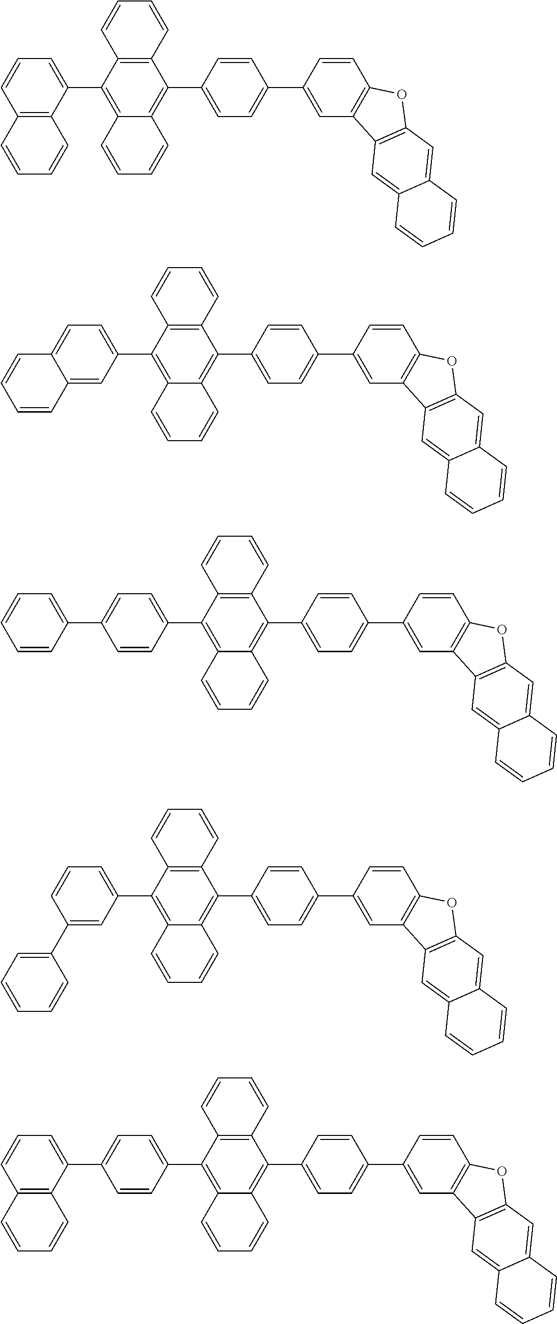 [SMILES: c1ccc2cc3c(cc2c1)oc1ccc(-c2ccc(-c4c5ccccc5c(-c5cccc6ccccc56)c5ccccc45)cc2)cc13 ; c1ccc2cc(-c3c4ccccc4c(-c4ccc(-c5ccc6oc7cc8ccccc8cc7c6c5)cc4)c4ccccc34)ccc2c1 ; c1ccc(-c2ccc(-c3c4ccccc4c(-c4ccc(-c5ccc6oc7cc8ccccc8cc7c6c5)cc4)c4ccccc34)cc2)cc1 ; c1ccc(-c2cccc(-c3c4ccccc4c(-c4ccc(-c5ccc6oc7cc8ccccc8cc7c6c5)cc4)c4ccccc34)c2)cc1 ; c1ccc2cc3c(cc2c1)oc1ccc(-c2ccc(-c4c5ccccc5c(-c5ccc(-c6cccc7ccccc67)cc5)c5ccccc45)cc2)cc13]

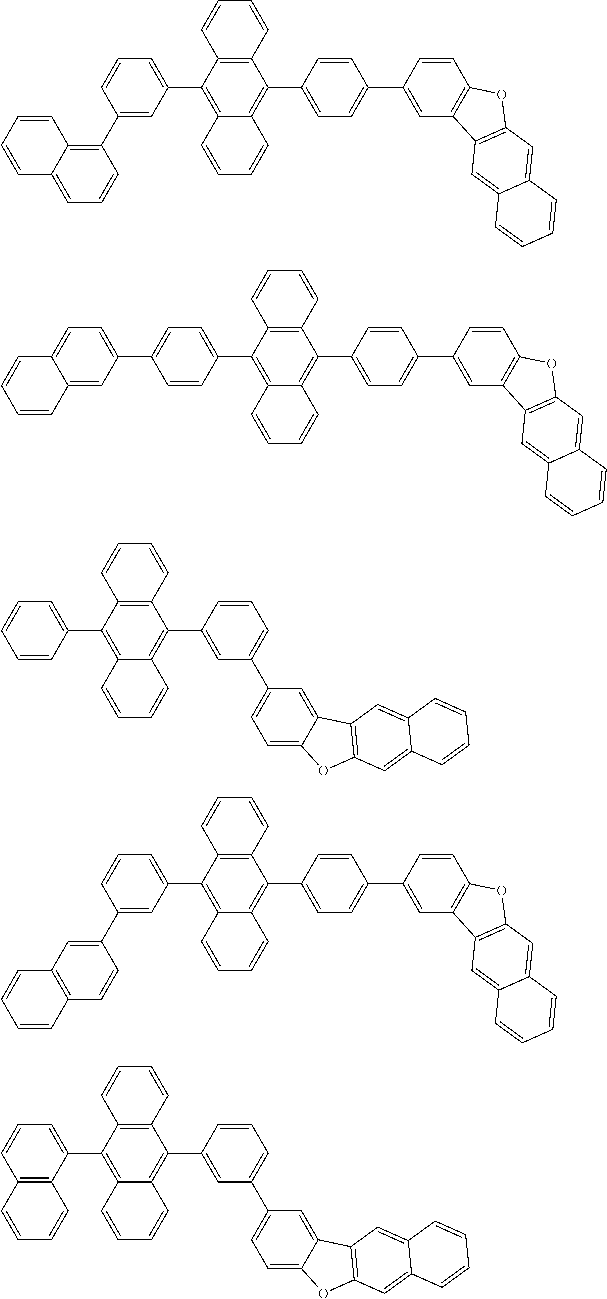 [SMILES: c1cc(-c2cccc3ccccc23)cc(-c2c3ccccc3c(-c3ccc(-c4ccc5oc6cc7ccccc7cc6c5c4)cc3)c3ccccc23)c1 ; c1ccc2cc(-c3ccc(-c4c5ccccc5c(-c5ccc(-c6ccc7oc8cc9ccccc9cc8c7c6)cc5)c5ccccc45)cc3)ccc2c1 ; c1ccc(-c2c3ccccc3c(-c3cccc(-c4ccc5oc6cc7ccccc7cc6c5c4)c3)c3ccccc23)cc1 ; c1cc(-c2ccc3ccccc3c2)cc(-c2c3ccccc3c(-c3ccc(-c4ccc5oc6cc7ccccc7cc6c5c4)cc3)c3ccccc23)c1 ; c1cc(-c2ccc3oc4cc5ccccc5cc4c3c2)cc(-c2c3ccccc3c(-c3cccc4ccccc34)c3ccccc23)c1]

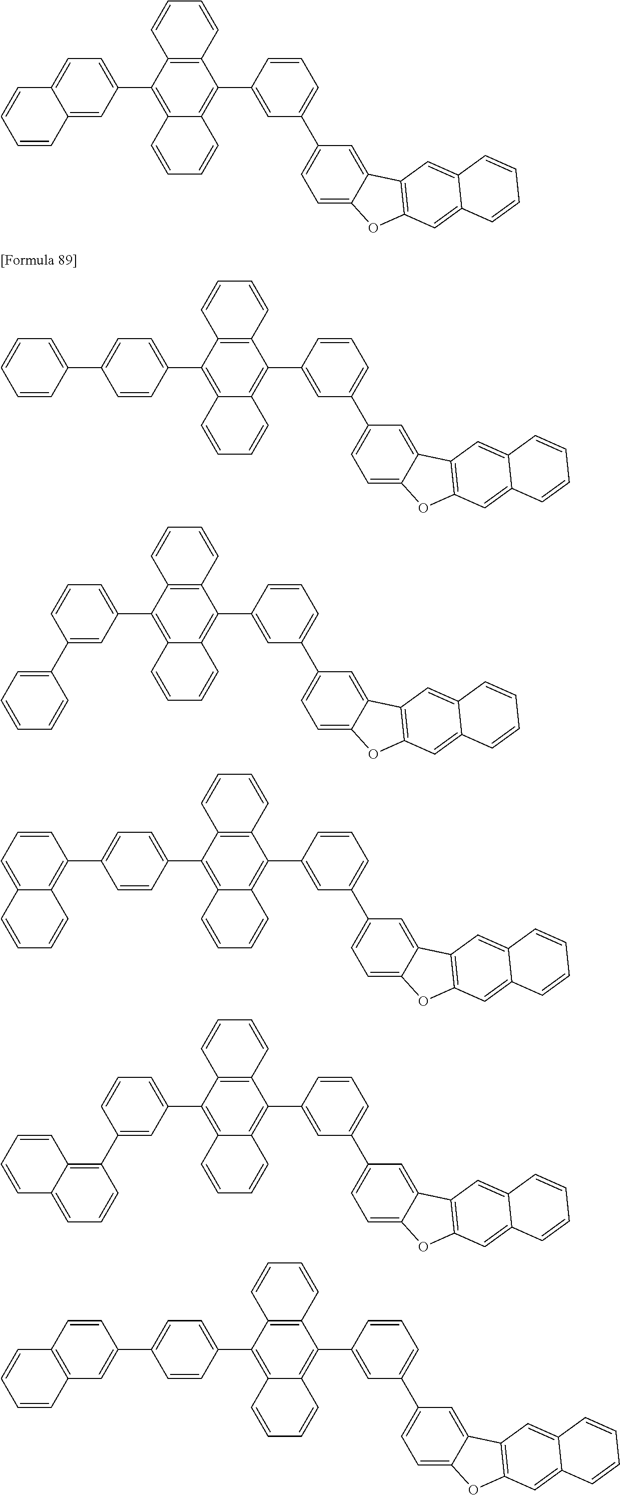

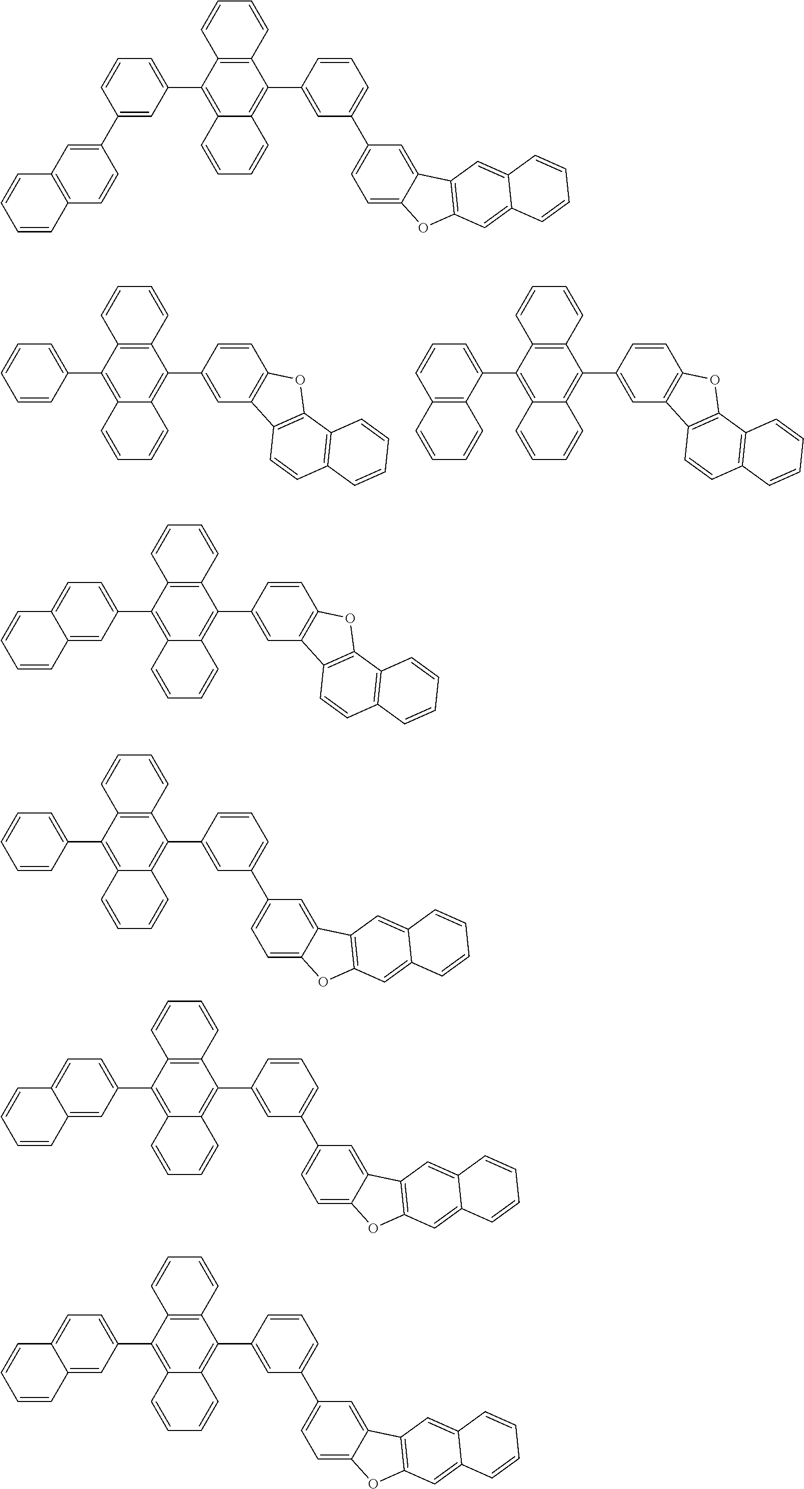

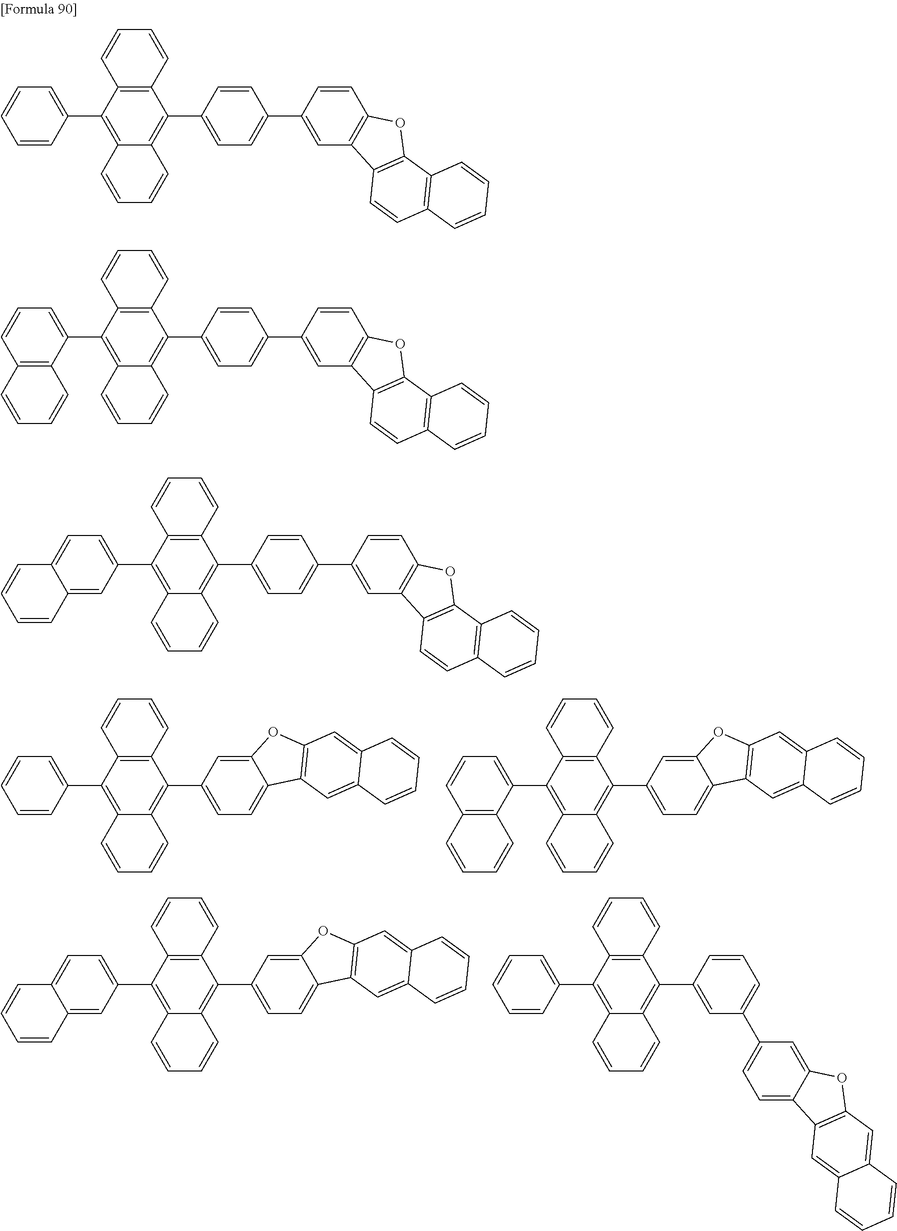

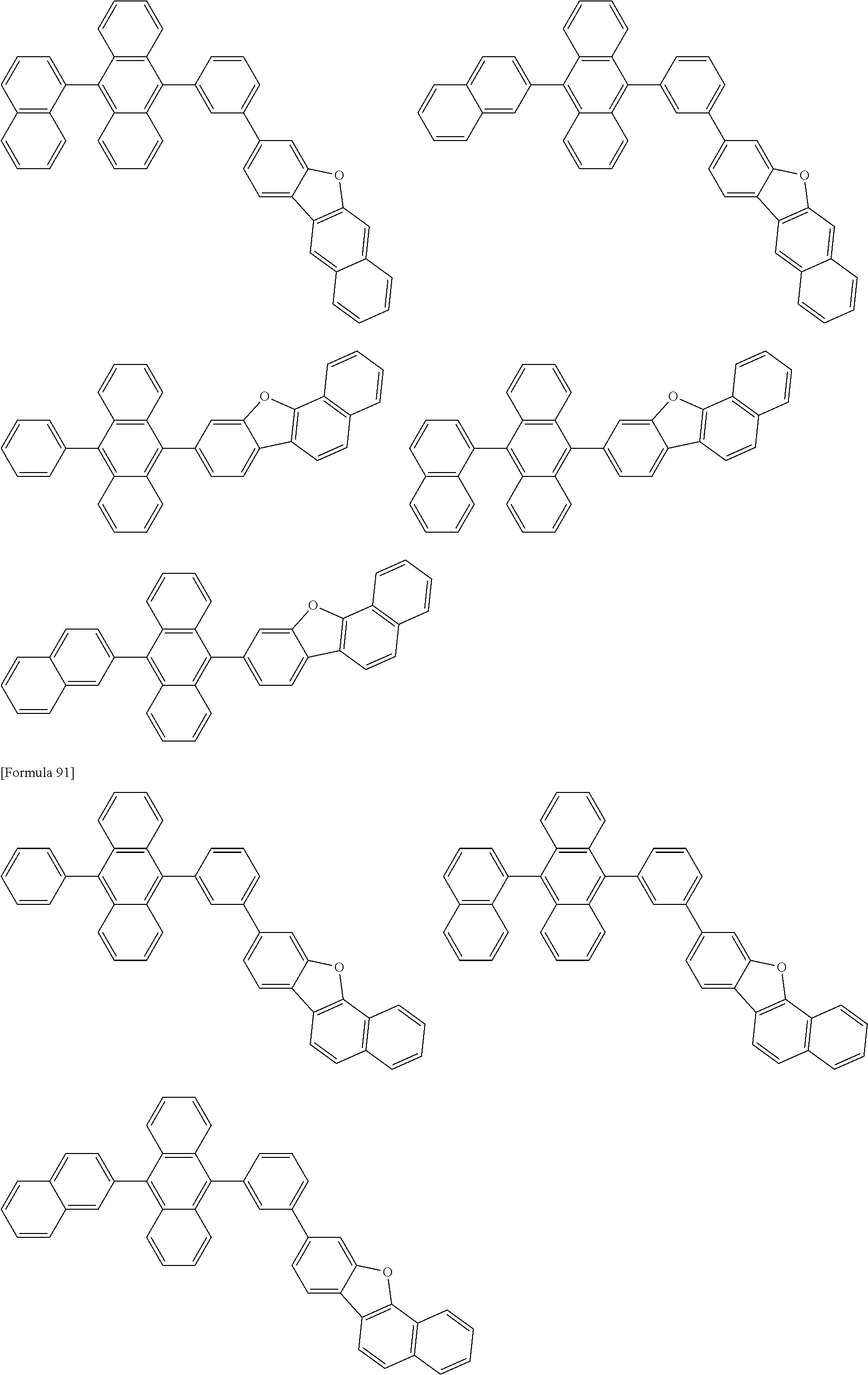

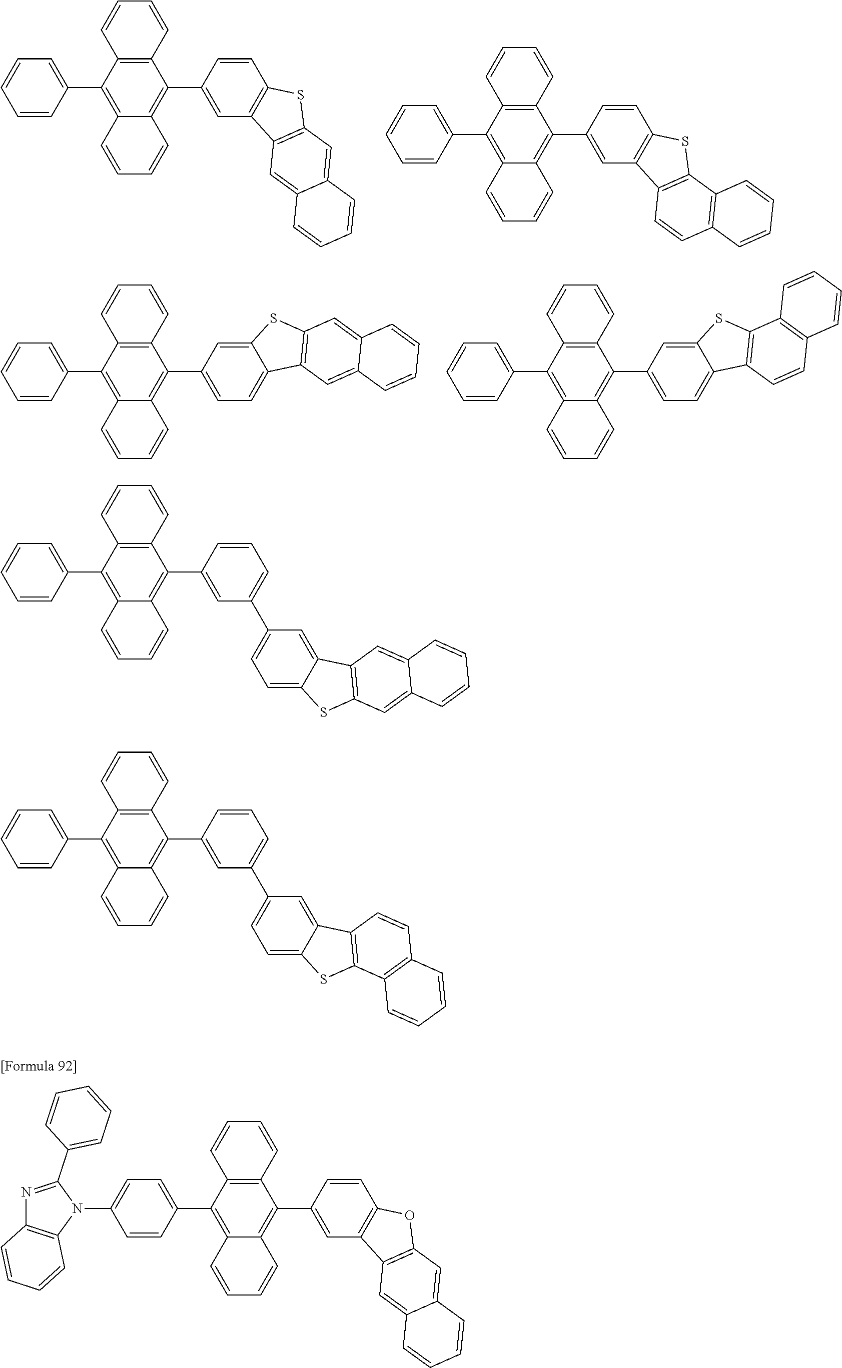

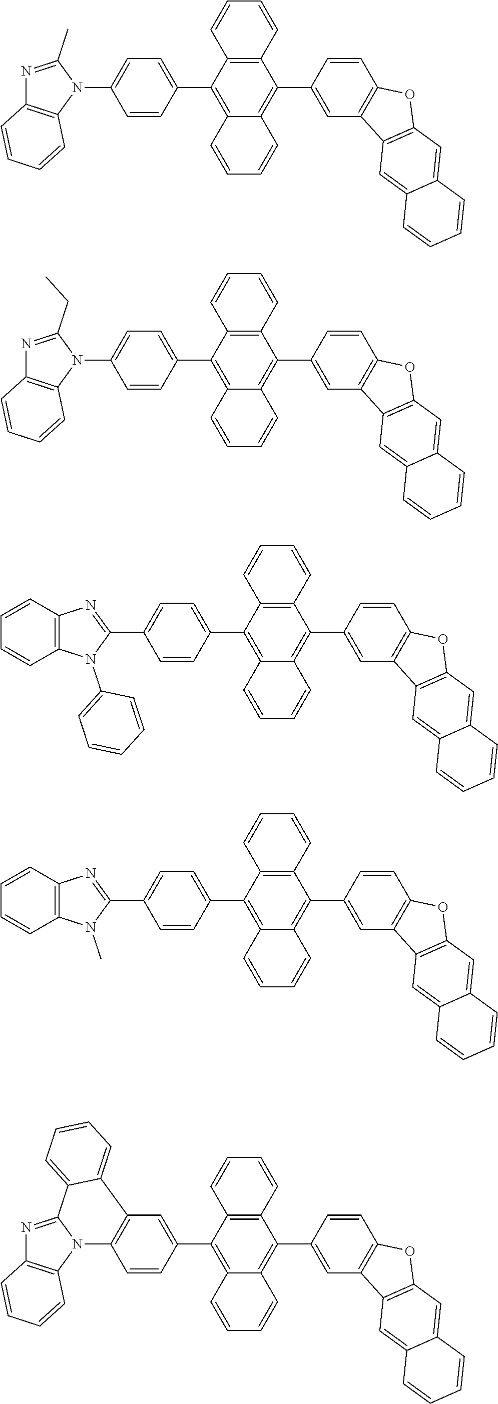 [SMILES: Cc1nc2ccccc2n1-c1ccc(-c2c3ccccc3c(-c3ccc4oc5cc6ccccc6cc5c4c3)c3ccccc23)cc1 ; CCc1nc2ccccc2n1-c1ccc(-c2c3ccccc3c(-c3ccc4oc5cc6ccccc6cc5c4c3)c3ccccc23)cc1 ; c1ccc(-n2c(-c3ccc(-c4c5ccccc5c(-c5ccc6oc7cc8ccccc8cc7c6c5)c5ccccc45)cc3)nc3ccccc32)cc1 ; Cn1c(-c2ccc(-c3c4ccccc4c(-c4ccc5oc6cc7ccccc7cc6c5c4)c4ccccc34)cc2)nc2ccccc21 ; c1ccc2cc3c(cc2c1)oc1ccc(-c2c4ccccc4c(-c4ccc5c(c4)c4ccccc4c4nc6ccccc6n54)c4ccccc24)cc13]

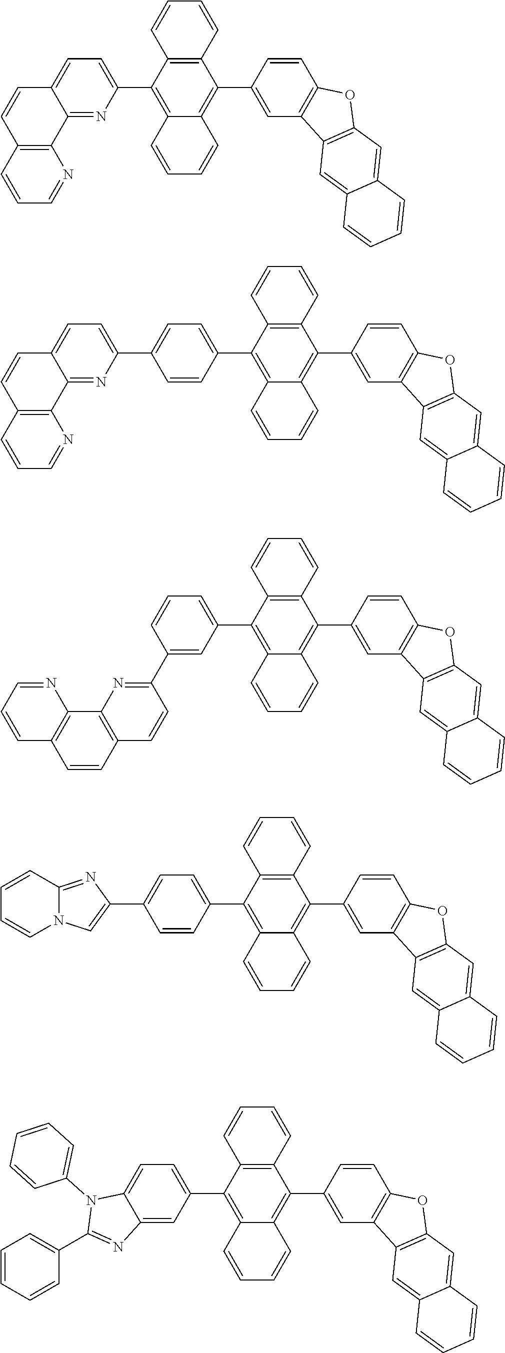 [SMILES: c1ccc2cc3c(cc2c1)oc1ccc(-c2c4ccccc4c(-c4ccc5ccc6cccnc6c5n4)c4ccccc24)cc13 ; c1ccc2cc3c(cc2c1)oc1ccc(-c2c4ccccc4c(-c4ccc(-c5ccc6ccc7cccnc7c6n5)cc4)c4ccccc24)cc13 ; c1cc(-c2ccc3ccc4cccnc4c3n2)cc(-c2c3ccccc3c(-c3ccc4oc5cc6ccccc6cc5c4c3)c3ccccc23)c1 ; c1ccc2cc3c(cc2c1)oc1ccc(-c2c4ccccc4c(-c4ccc(-c5cn6ccccc6n5)cc4)c4ccccc24)cc13 ; c1ccc(-c2nc3cc(-c4c5ccccc5c(-c5ccc6oc7cc8ccccc8cc7c6c5)c5ccccc45)ccc3n2-c2ccccc2)cc1]

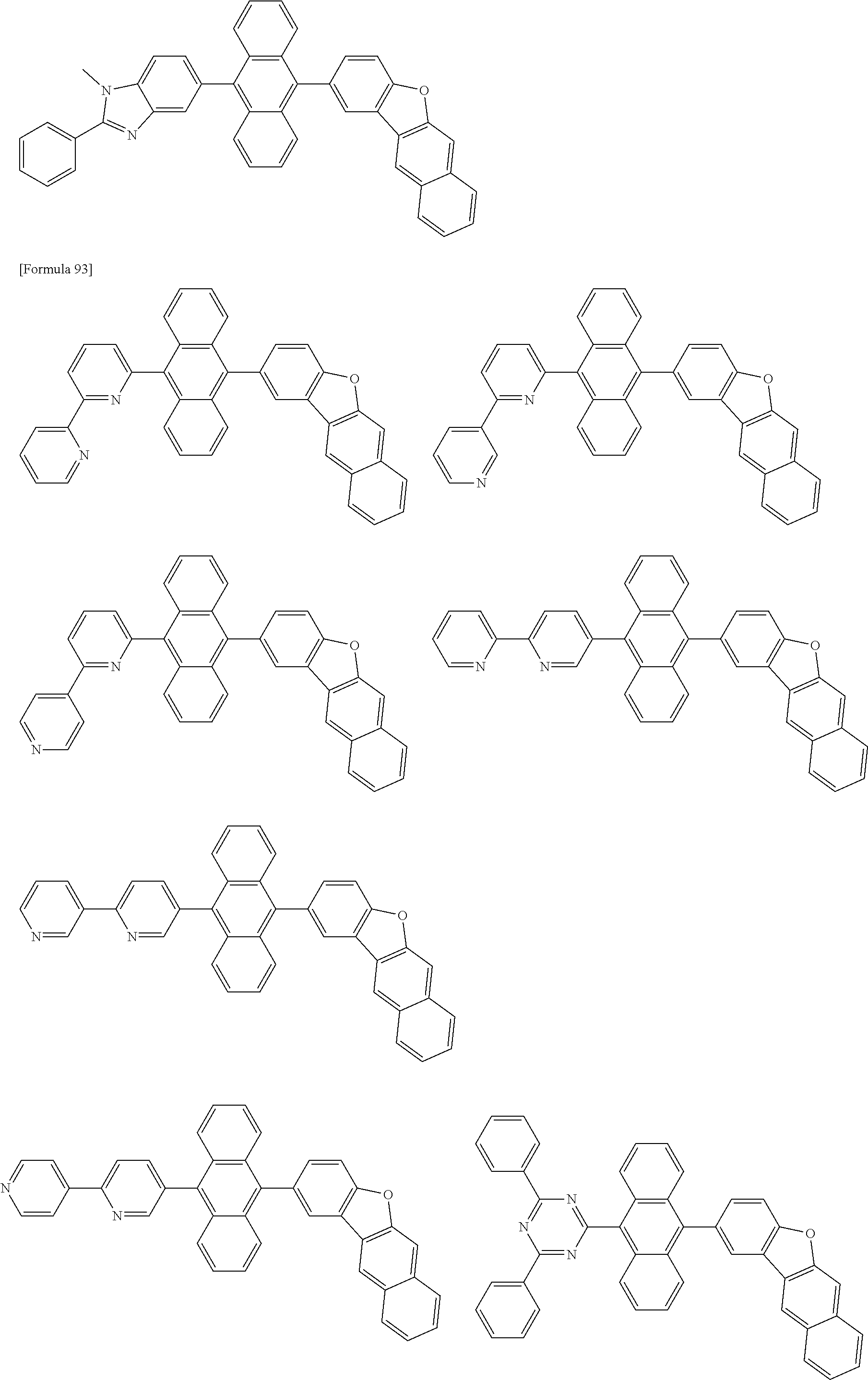

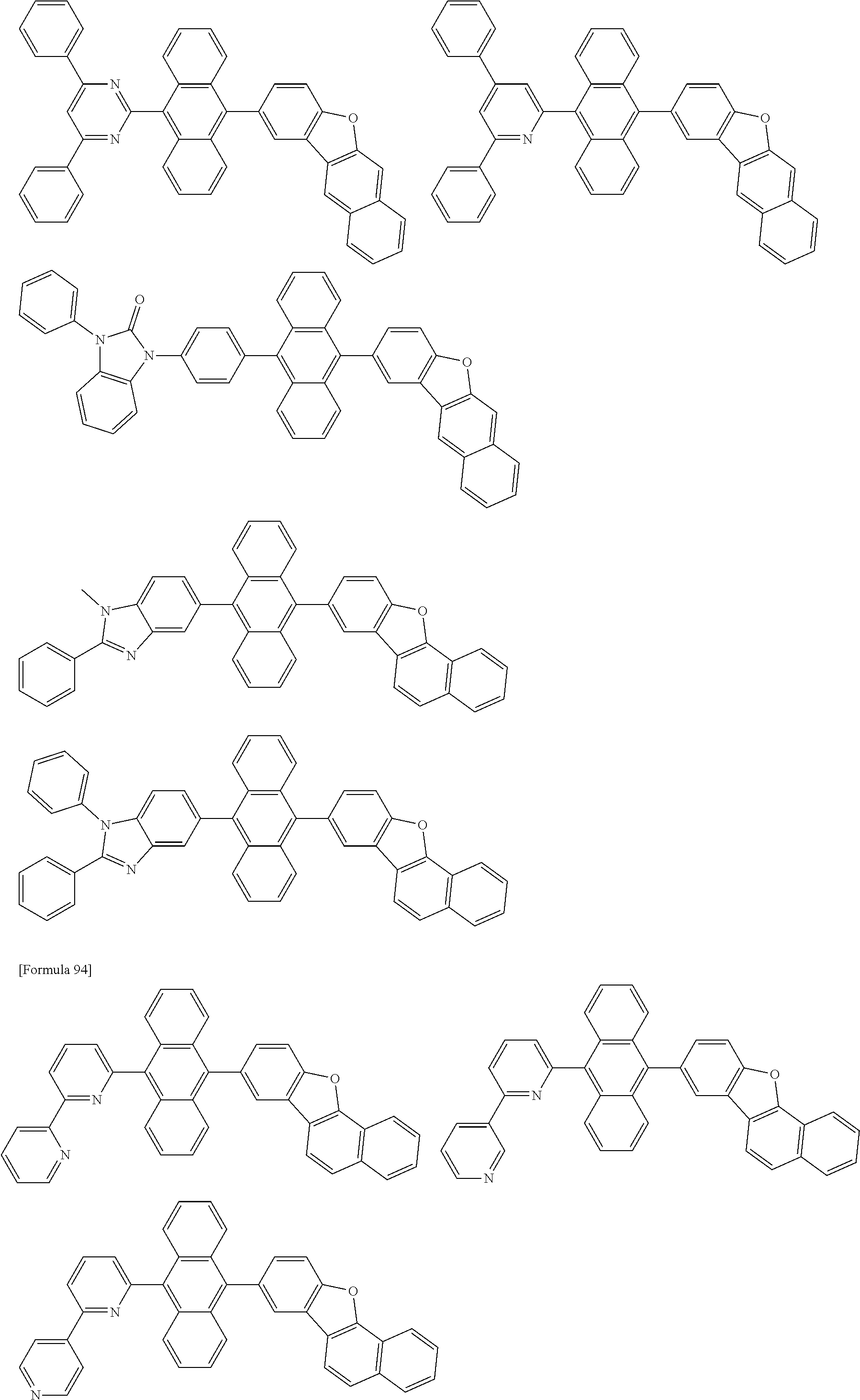

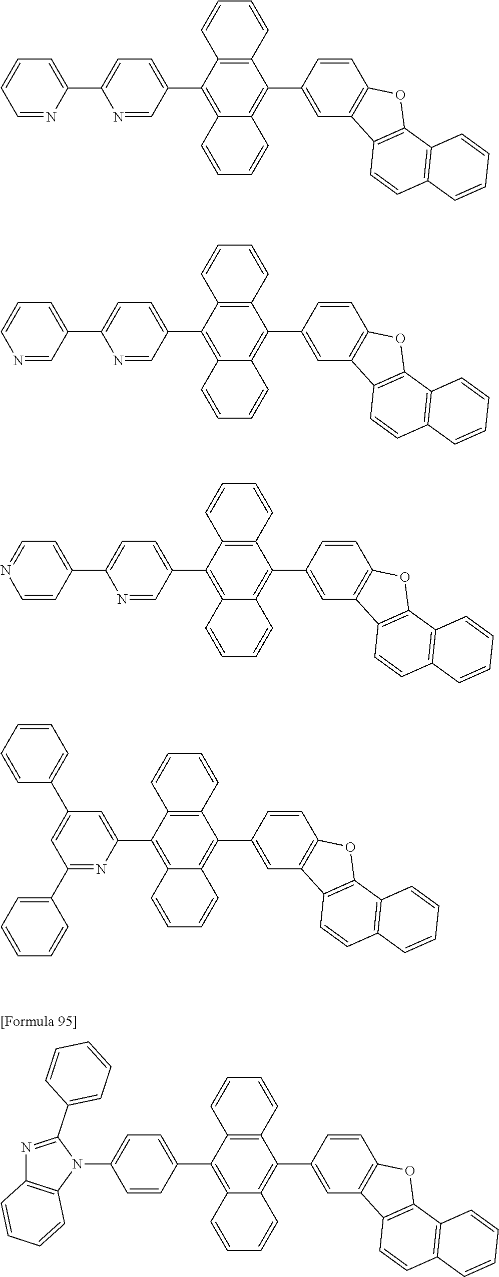 [SMILES: c1ccc(-c2ccc(-c3c4ccccc4c(-c4ccc5oc6c7ccccc7ccc6c5c4)c4ccccc34)cn2)nc1 ; c1cncc(-c2ccc(-c3c4ccccc4c(-c4ccc5oc6c7ccccc7ccc6c5c4)c4ccccc34)cn2)c1 ; c1ccc2c(c1)ccc1c3cc(-c4c5ccccc5c(-c5ccc(-c6ccncc6)nc5)c5ccccc45)ccc3oc21 ; c1ccc(-c2cc(-c3ccccc3)nc(-c3c4ccccc4c(-c4ccc5oc6c7ccccc7ccc6c5c4)c4ccccc34)c2)cc1 ; c1ccc(-c2nc3ccccc3n2-c2ccc(-c3c4ccccc4c(-c4ccc5oc6c7ccccc7ccc6c5c4)c4ccccc34)cc2)cc1]

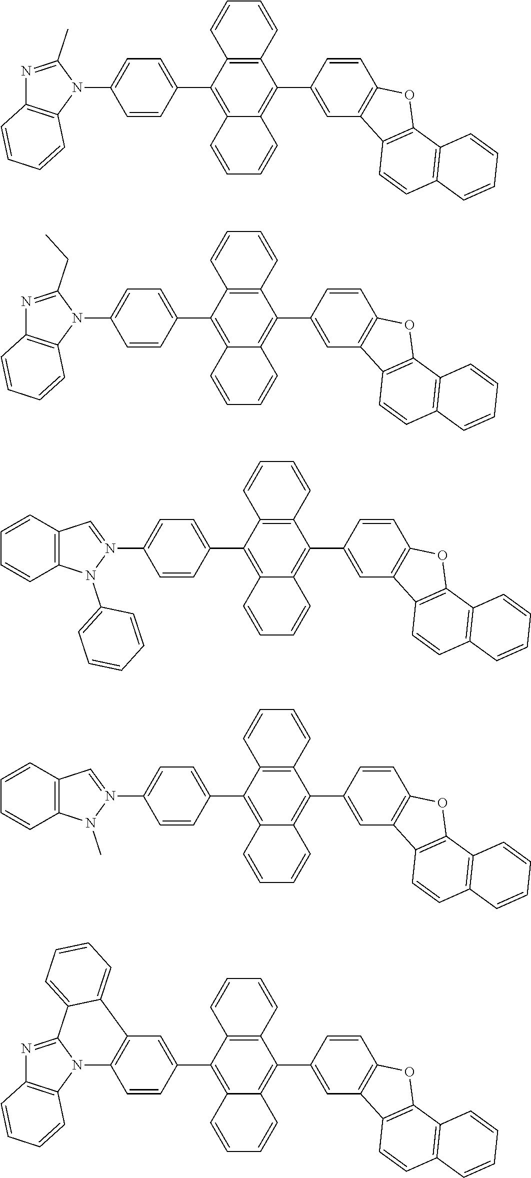

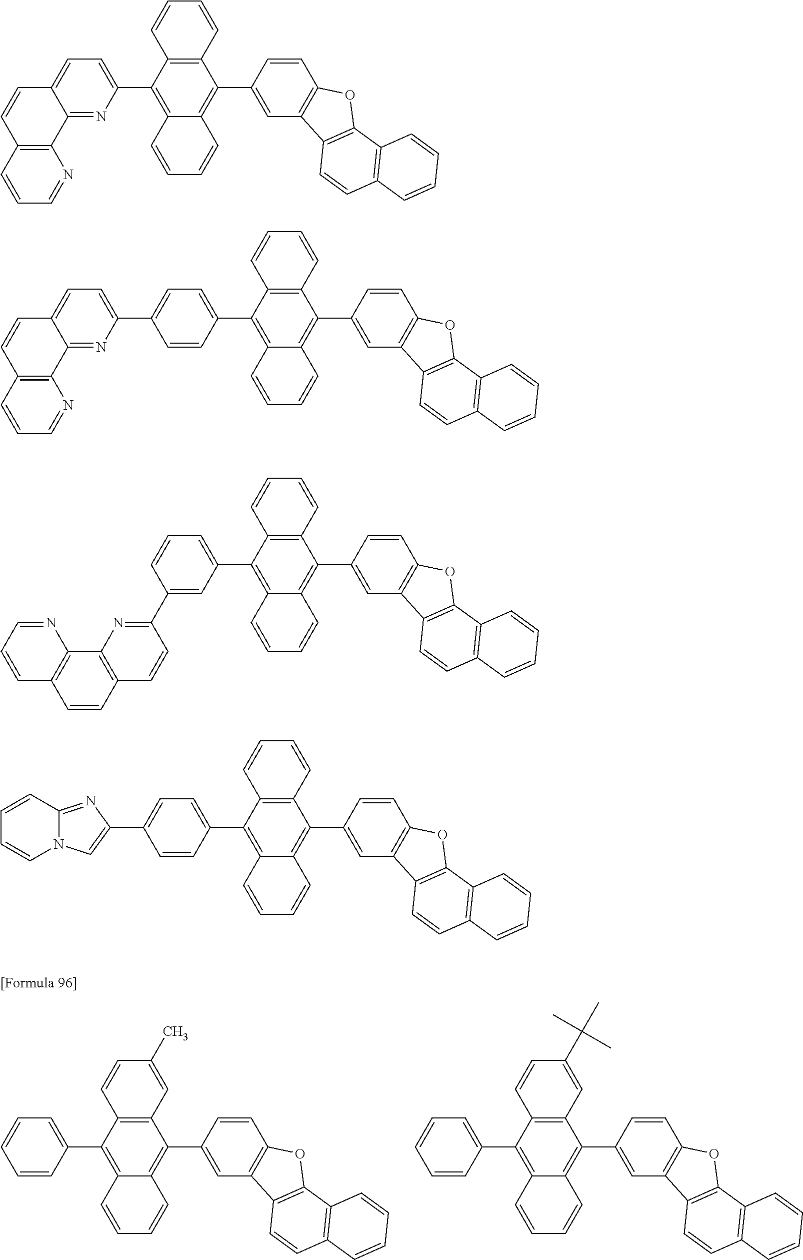 [SMILES: c1ccc2c(c1)ccc1c3cc(-c4c5ccccc5c(-c5ccc6ccc7cccnc7c6n5)c5ccccc45)ccc3oc21 ; c1ccc2c(c1)ccc1c3cc(-c4c5ccccc5c(-c5ccc(-c6ccc7ccc8cccnc8c7n6)cc5)c5ccccc45)ccc3oc21 ; c1cc(-c2ccc3ccc4cccnc4c3n2)cc(-c2c3ccccc3c(-c3ccc4oc5c6ccccc6ccc5c4c3)c3ccccc23)c1 ; c1ccc2c(c1)ccc1c3cc(-c4c5ccccc5c(-c5ccc(-c6cn7ccccc7n6)cc5)c5ccccc45)ccc3oc21 ; Cc1ccc2c(-c3ccccc3)c3ccccc3c(-c3ccc4oc5c6ccccc6ccc5c4c3)c2c1 ; CC(C)(C)c1ccc2c(-c3ccccc3)c3ccccc3c(-c3ccc4oc5c6ccccc6ccc5c4c3)c2c1]

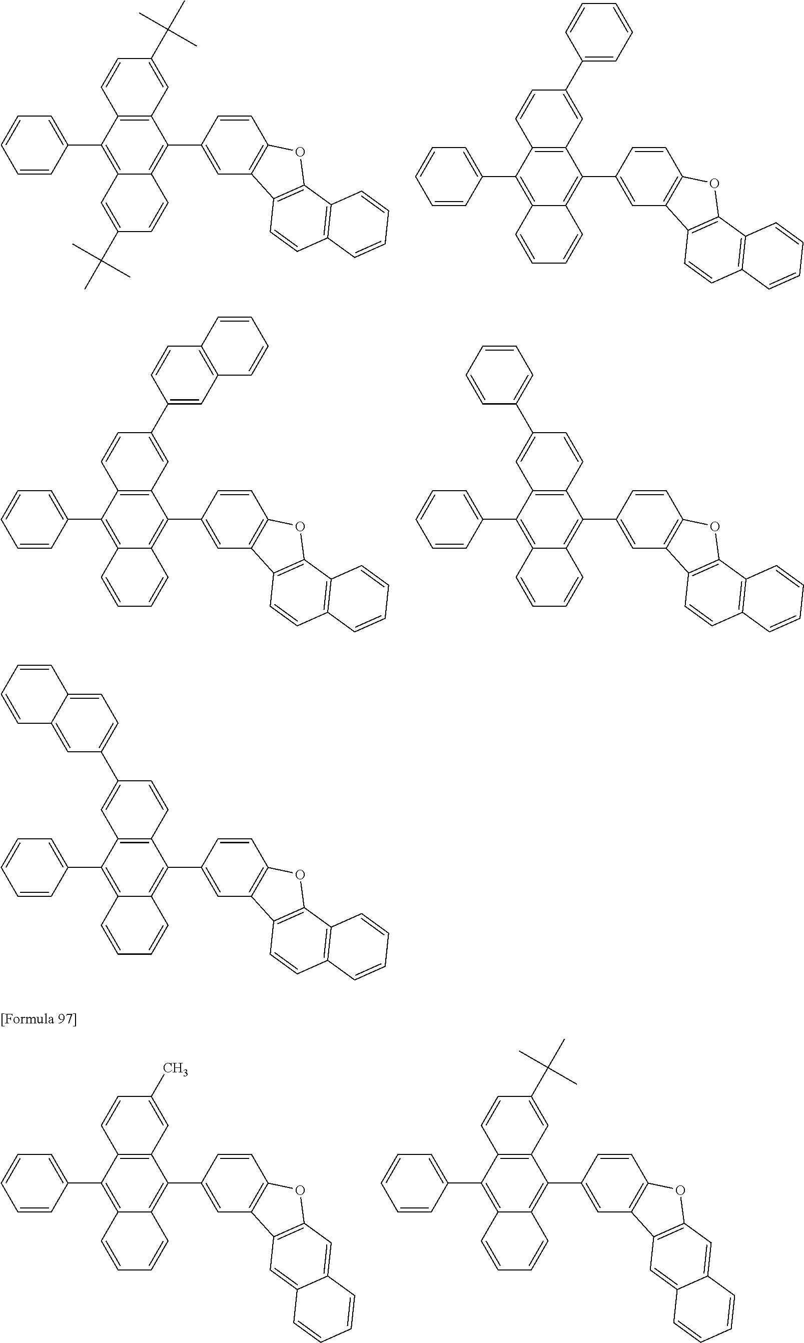

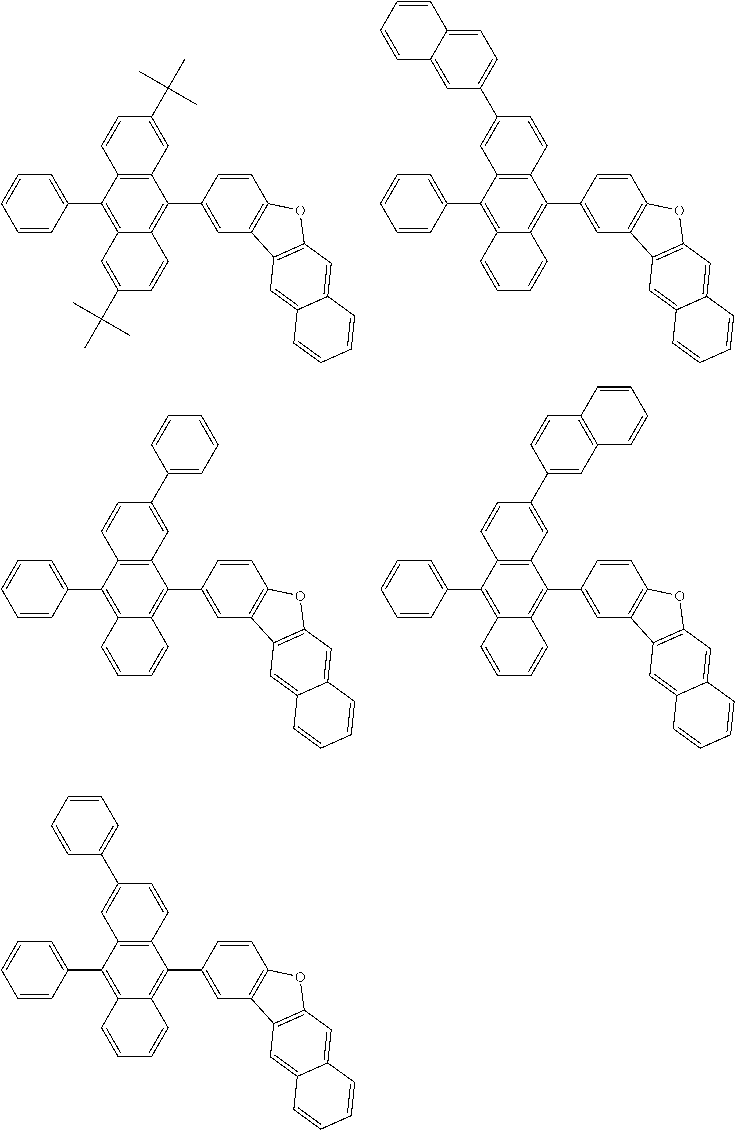 [SMILES: CC(C)(C)c1ccc2c(-c3ccc4oc5cc6ccccc6cc5c4c3)c3cc(C(C)(C)C)ccc3c(-c3ccccc3)c2c1 ; c1ccc(-c2c3ccccc3c(-c3ccc4oc5cc6ccccc6cc5c4c3)c3ccc(-c4ccc5ccccc5c4)cc23)cc1 ; c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1 ; c1ccc(-c2ccc3c(-c4ccc5oc6cc7ccccc7cc6c5c4)c4ccccc4c(-c4ccccc4)c3c2)cc1]

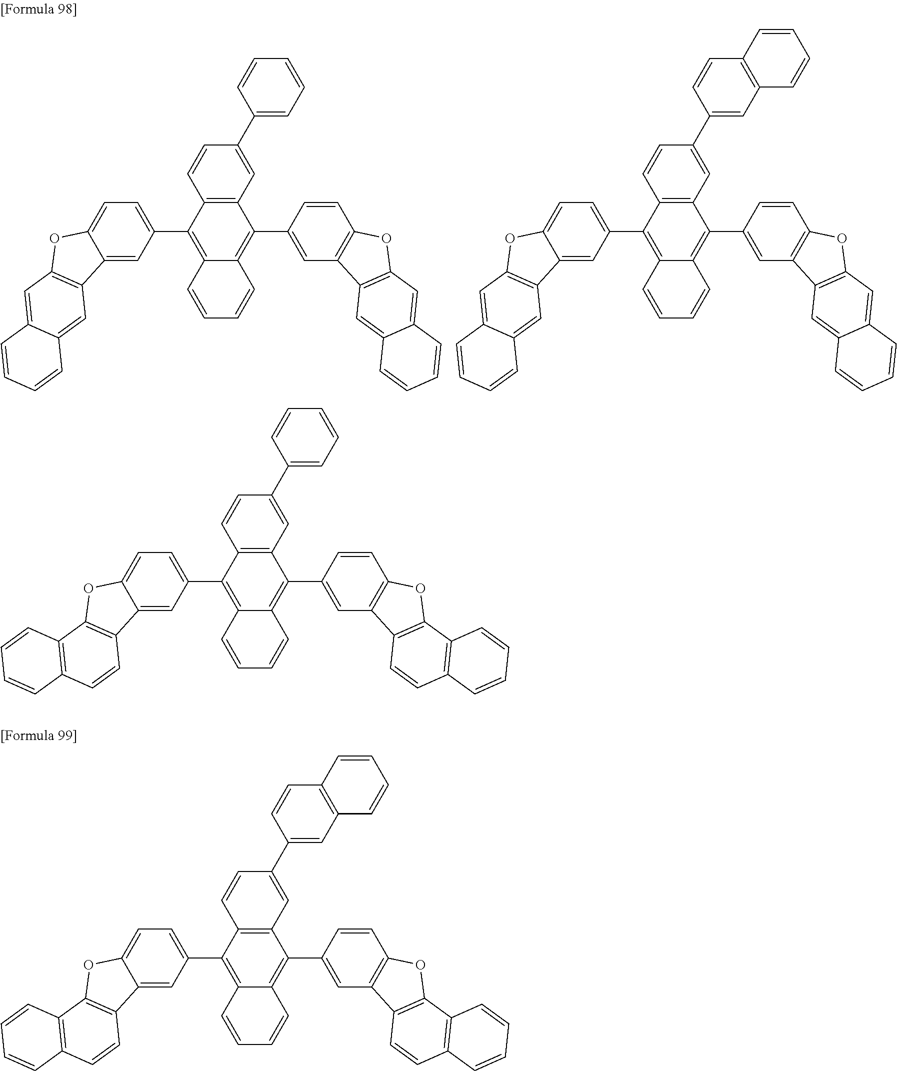

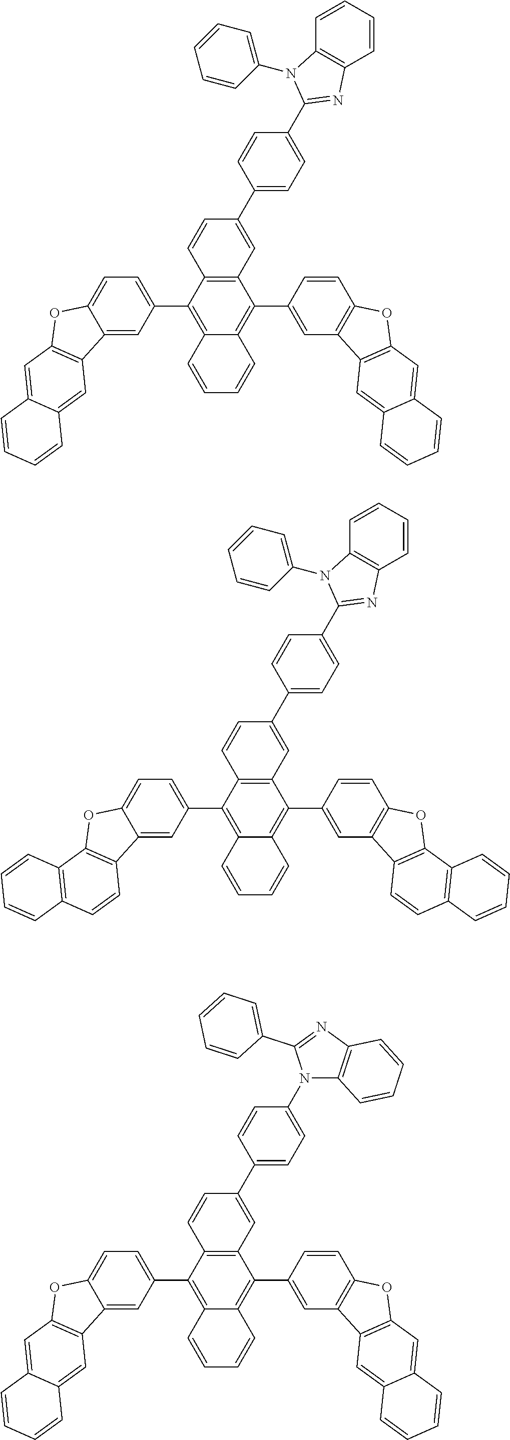 [SMILES: c1ccc(-n2c(-c3ccc(-c4ccc5c(-c6ccc7oc8cc9ccccc9cc8c7c6)c6ccccc6c(-c6ccc7oc8cc9ccccc9cc8c7c6)c5c4)cc3)nc3ccccc32)cc1 ; c1ccc(-n2c(-c3ccc(-c4ccc5c(-c6ccc7oc8c9ccccc9ccc8c7c6)c6ccccc6c(-c6ccc7oc8c9ccccc9ccc8c7c6)c5c4)cc3)nc3ccccc32)cc1 ; c1ccc(-c2nc3ccccc3n2-c2ccc(-c3ccc4c(-c5ccc6oc7cc8ccccc8cc7c6c5)c5ccccc5c(-c5ccc6oc7cc8ccccc8cc7c6c5)c4c3)cc2)cc1]

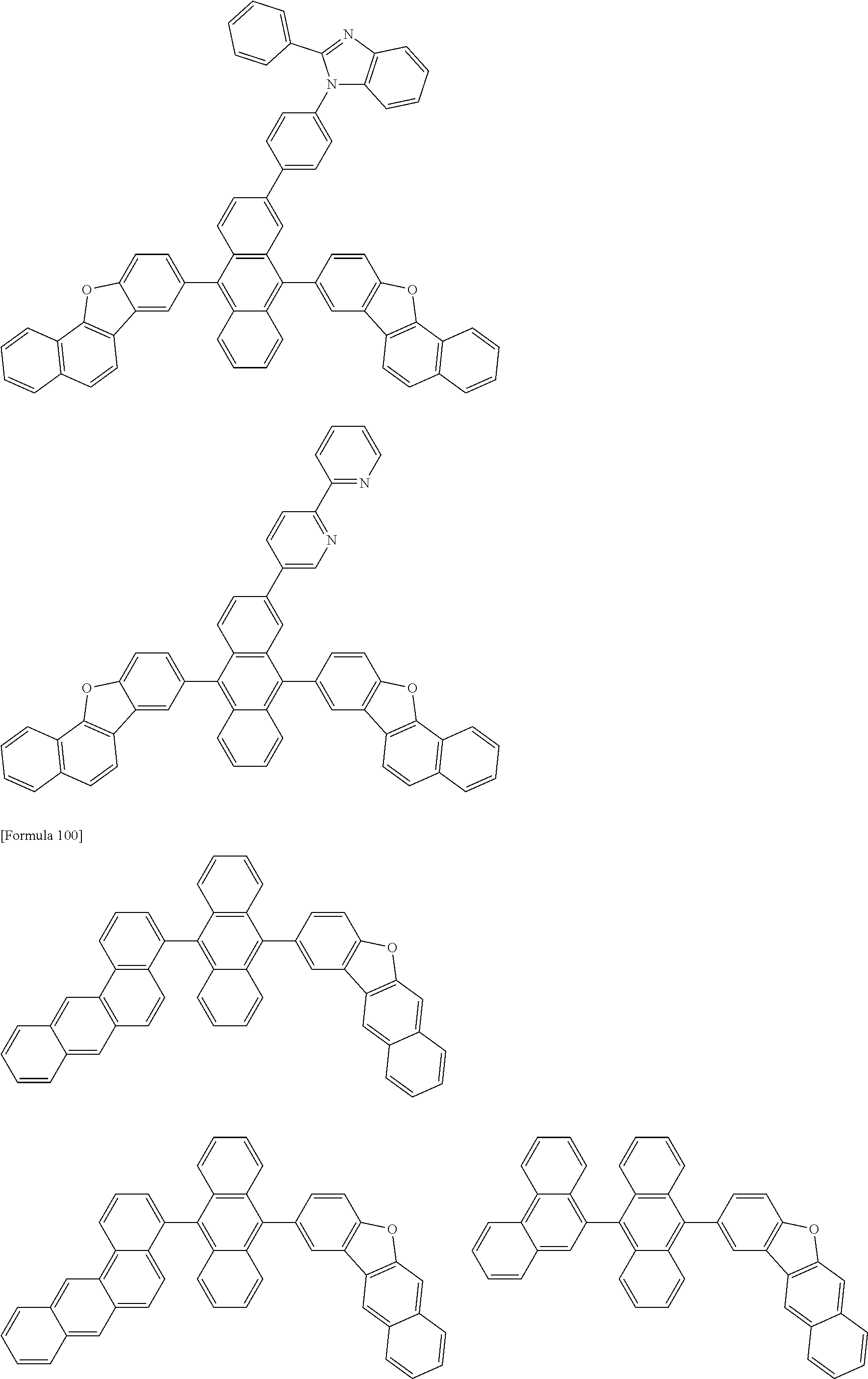 [SMILES: c1ccc(-c2nc3ccccc3n2-c2ccc(-c3ccc4c(-c5ccc6oc7c8ccccc8ccc7c6c5)c5ccccc5c(-c5ccc6oc7c8ccccc8ccc7c6c5)c4c3)cc2)cc1 ; c1ccc(-c2ccc(-c3ccc4c(-c5ccc6oc7c8ccccc8ccc7c6c5)c5ccccc5c(-c5ccc6oc7c8ccccc8ccc7c6c5)c4c3)cn2)nc1 ; c1ccc2cc3c(ccc4c(-c5c6ccccc6c(-c6ccc7oc8cc9ccccc9cc8c7c6)c6ccccc56)cccc43)cc2c1 ; c1ccc2cc3c(ccc4c(-c5c6ccccc6c(-c6ccc7oc8cc9ccccc9cc8c7c6)c6ccccc56)cccc43)cc2c1 ; c1ccc2cc3c(cc2c1)oc1ccc(-c2c4ccccc4c(-c4cc5ccccc5c5ccccc45)c4ccccc24)cc13]

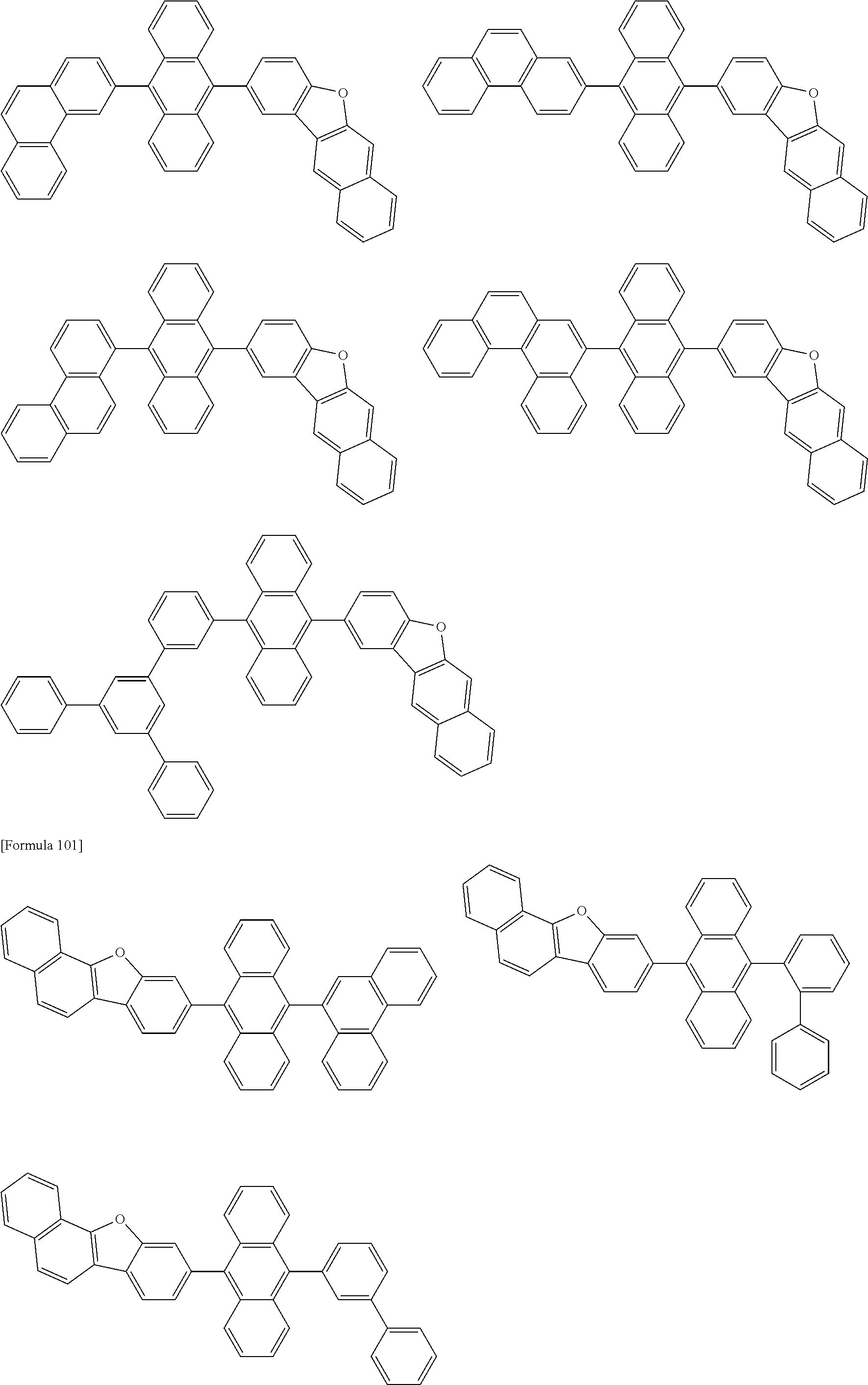

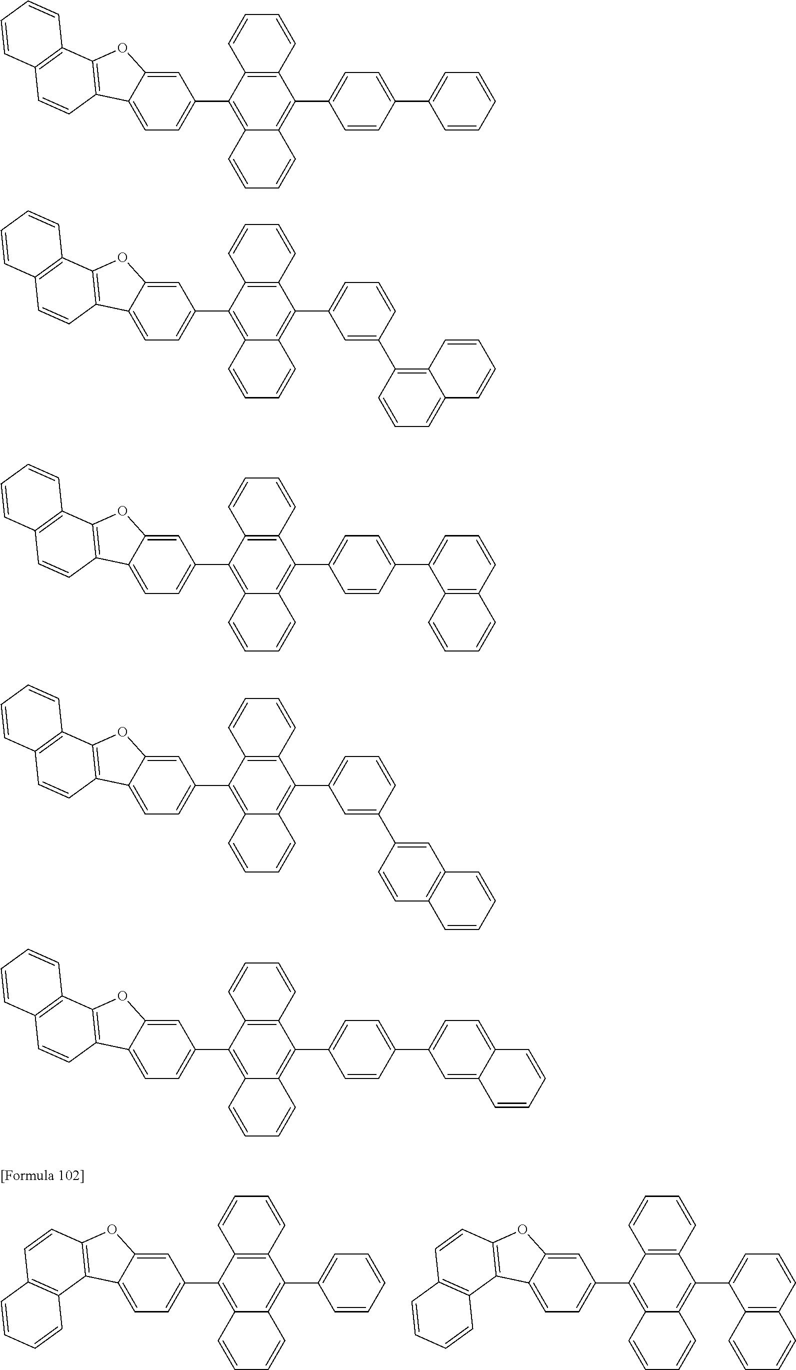

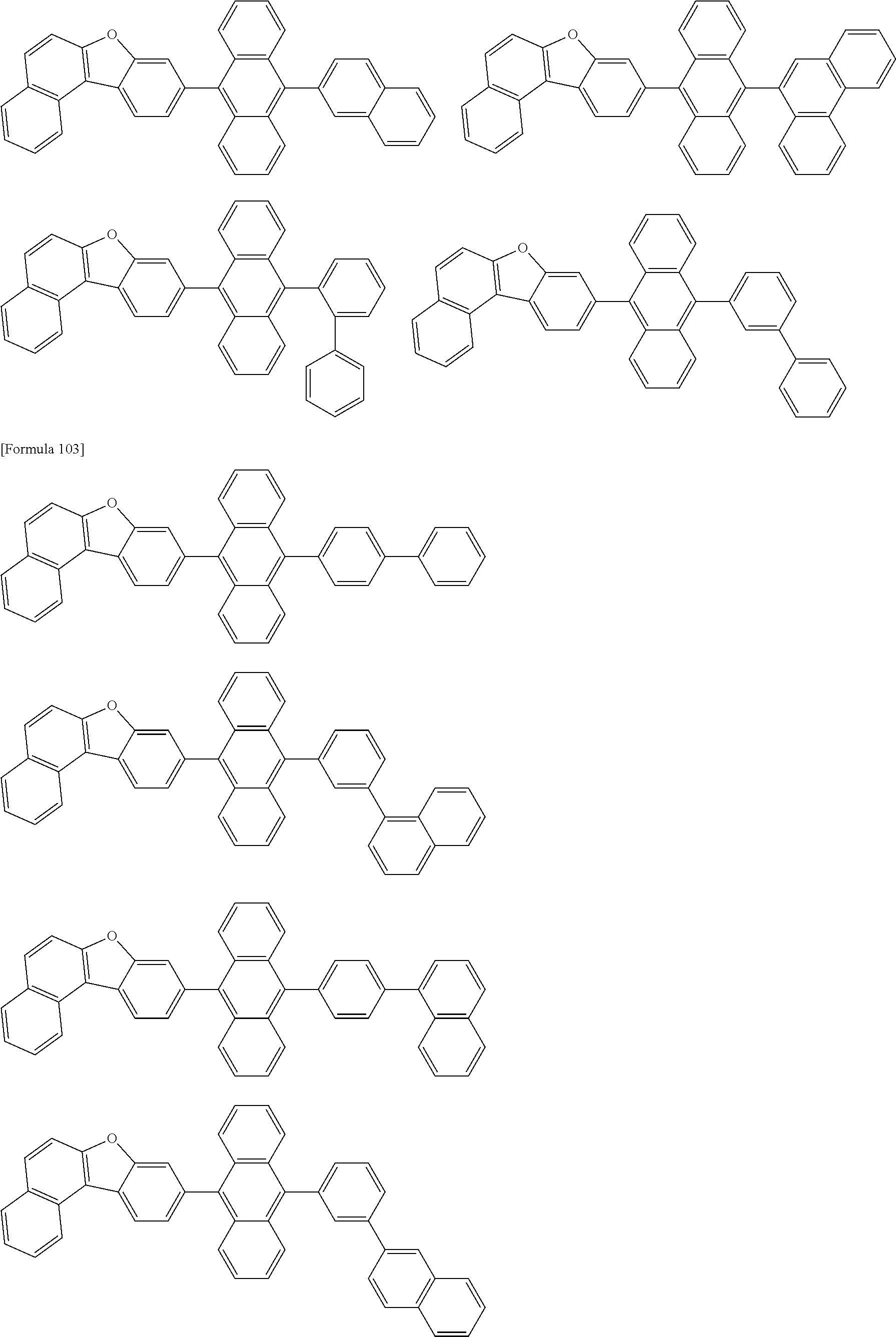

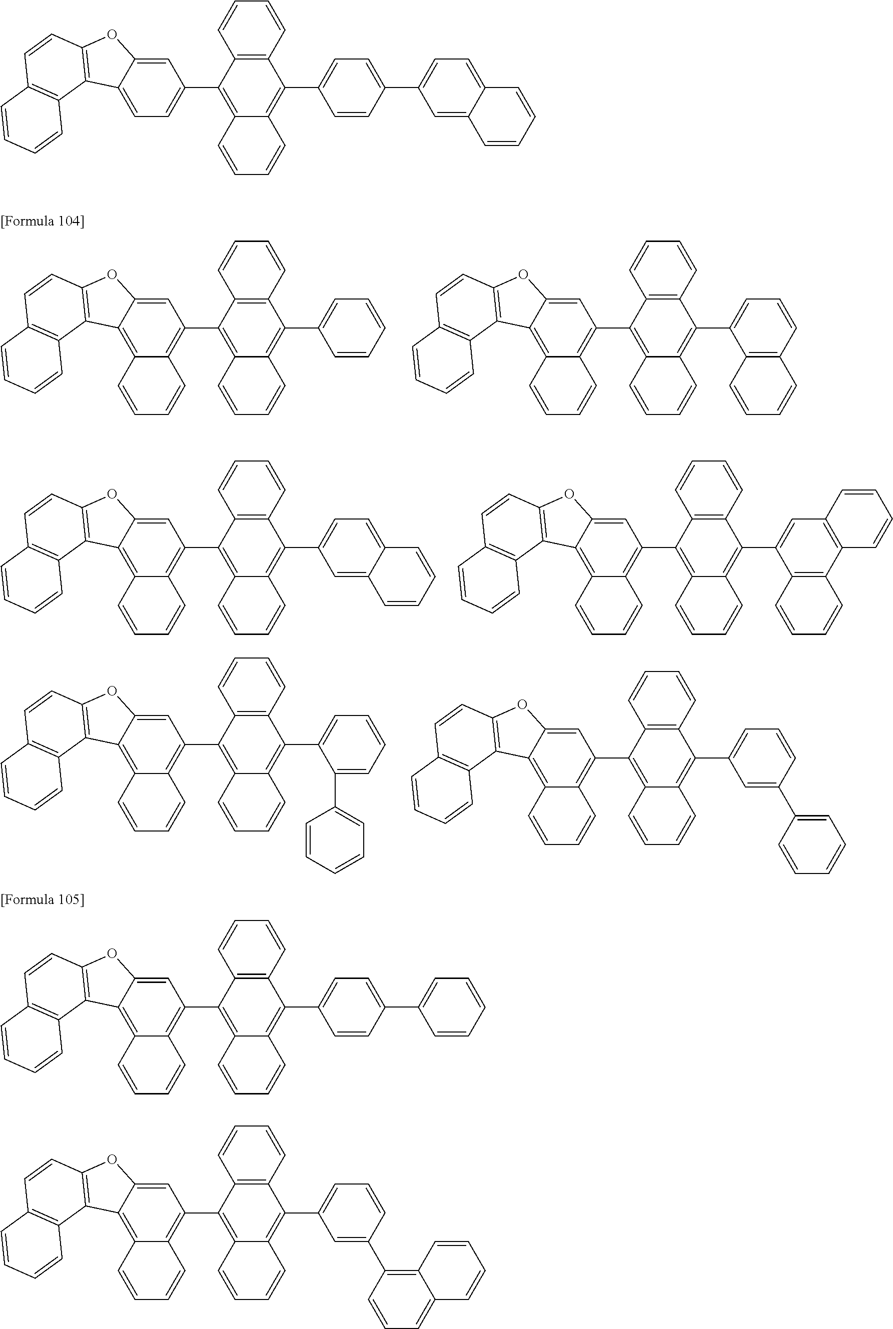

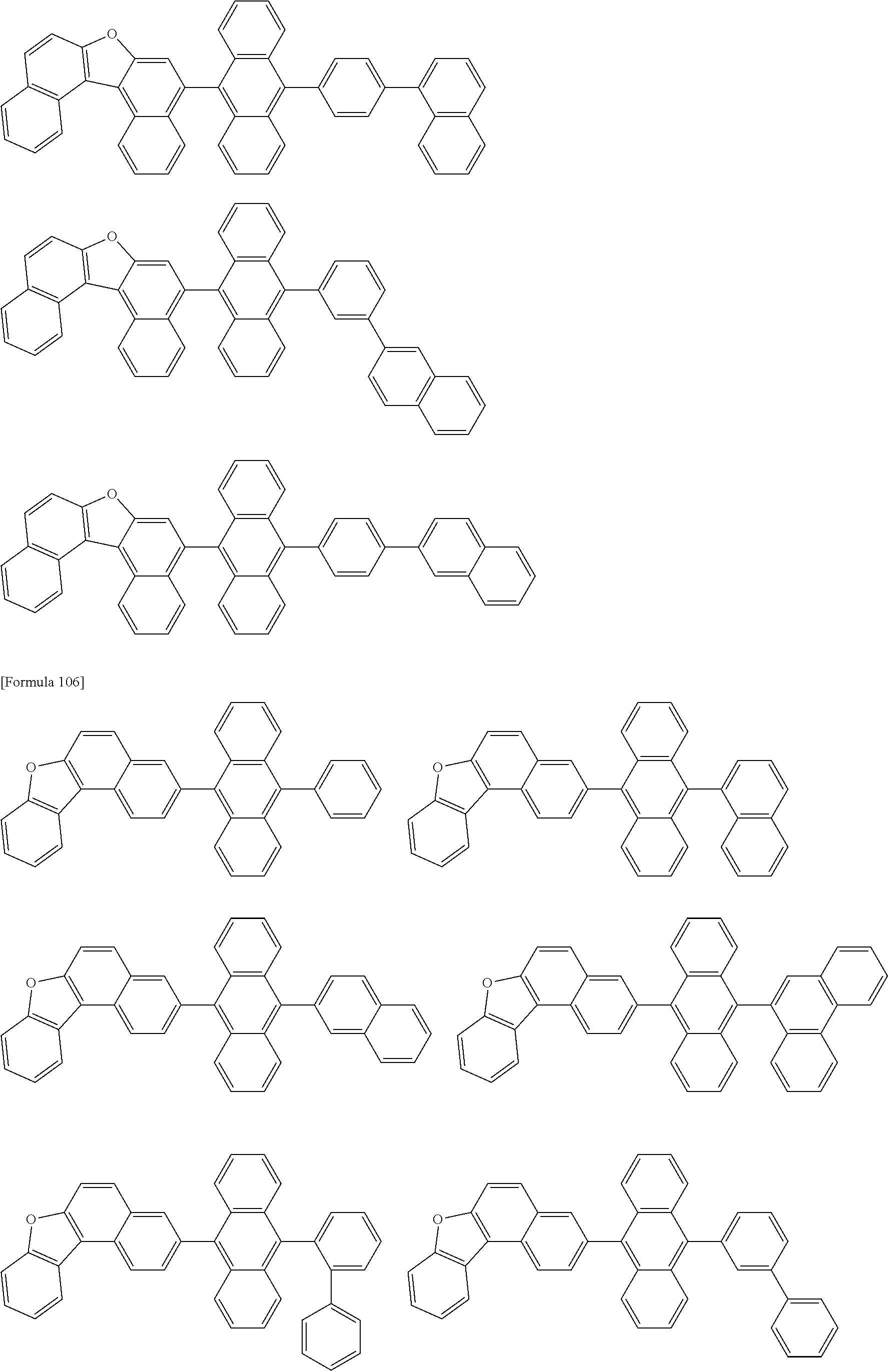

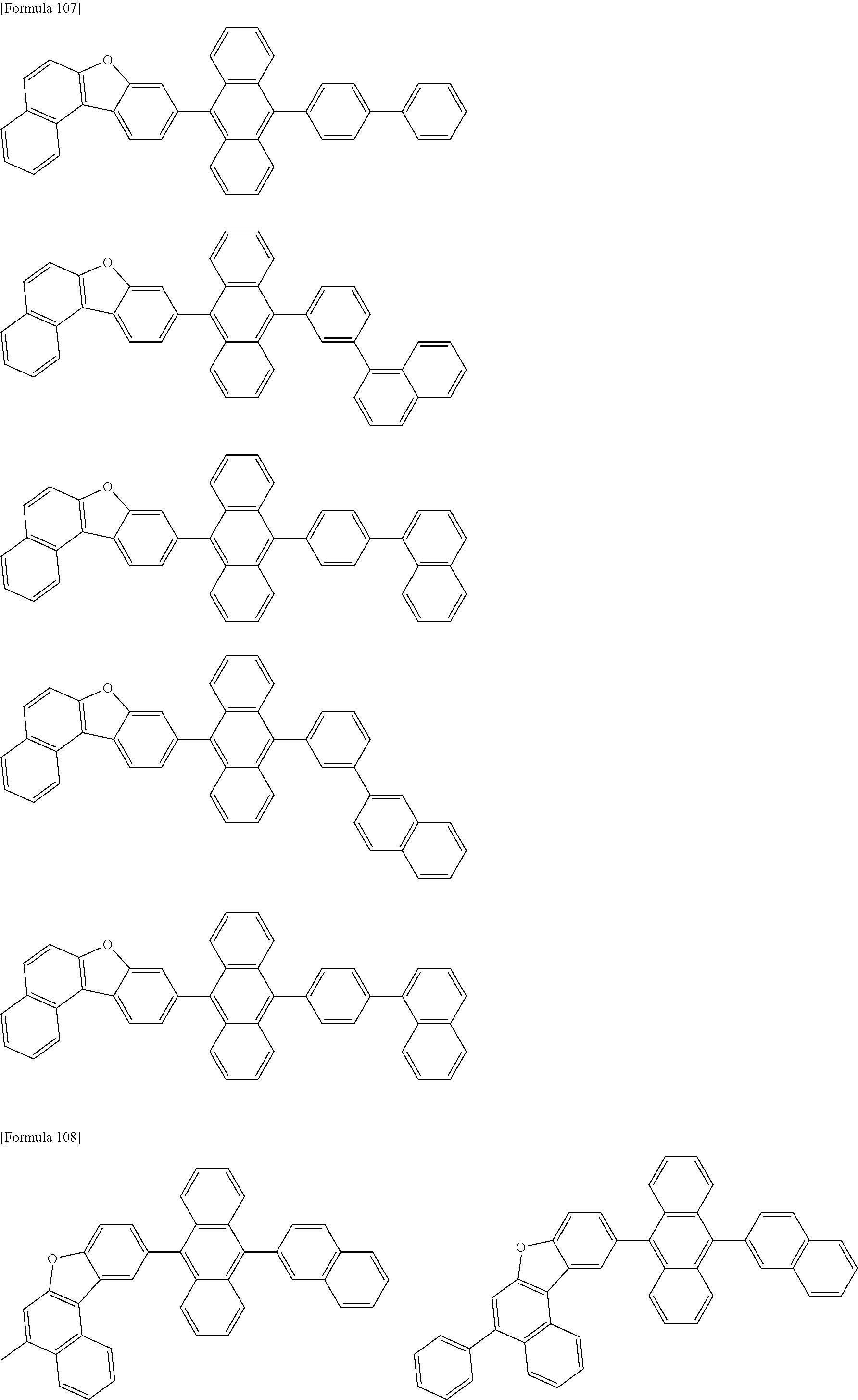

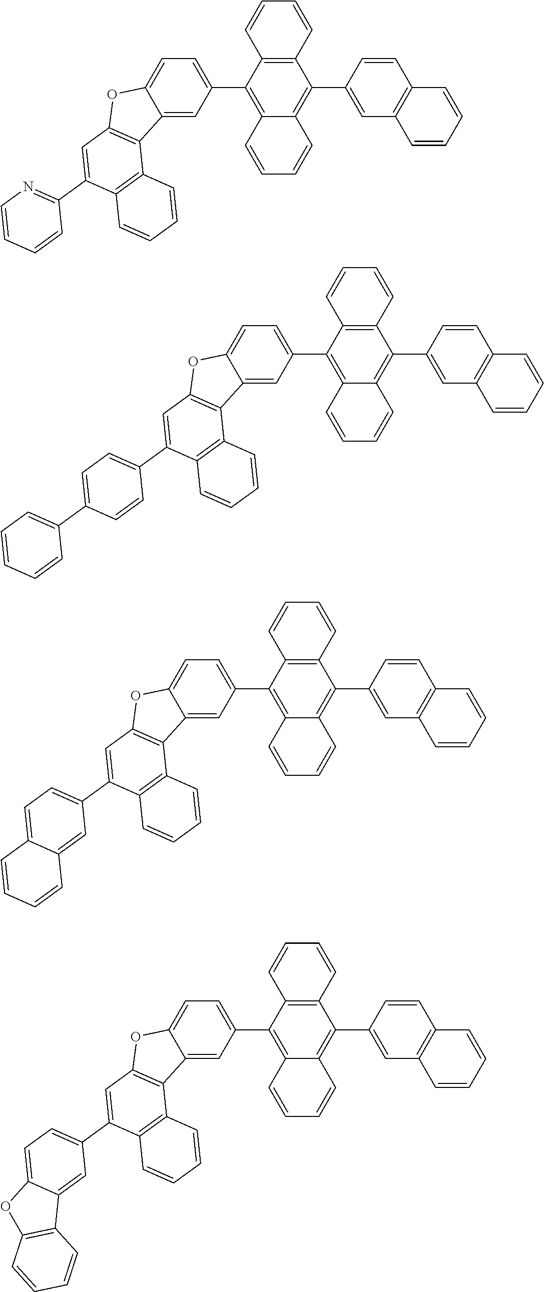 [SMILES: c1ccc(-c2cc3oc4ccc(-c5c6ccccc6c(-c6ccc7ccccc7c6)c6ccccc56)cc4c3c3ccccc23)nc1 ; c1ccc(-c2ccc(-c3cc4oc5ccc(-c6c7ccccc7c(-c7ccc8ccccc8c7)c7ccccc67)cc5c4c4ccccc34)cc2)cc1 ; c1ccc2cc(-c3c4ccccc4c(-c4ccc5oc6cc(-c7ccc8ccccc8c7)c7ccccc7c6c5c4)c4ccccc34)ccc2c1 ; c1ccc2cc(-c3c4ccccc4c(-c4ccc5oc6cc(-c7ccc8oc9ccccc9c8c7)c7ccccc7c6c5c4)c4ccccc34)ccc2c1]

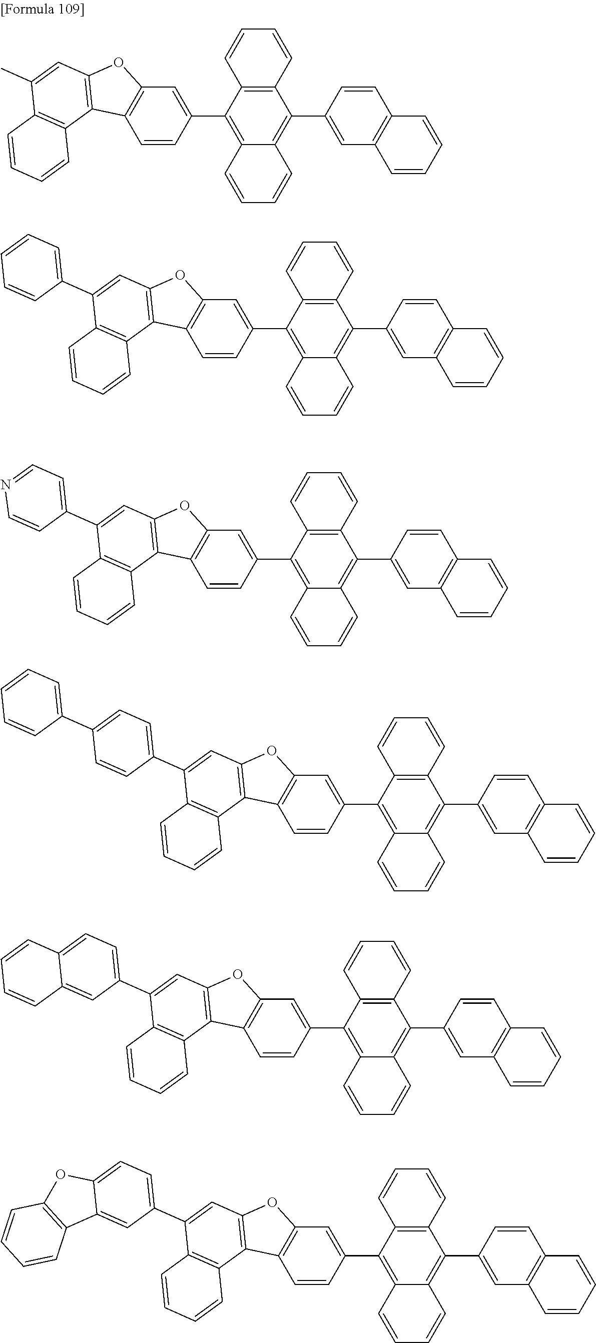 [SMILES: Cc1cc2oc3cc(-c4c5ccccc5c(-c5ccc6ccccc6c5)c5ccccc45)ccc3c2c2ccccc12 ; c1ccc(-c2cc3oc4cc(-c5c6ccccc6c(-c6ccc7ccccc7c6)c6ccccc56)ccc4c3c3ccccc23)cc1 ; c1ccc2cc(-c3c4ccccc4c(-c4ccc5c(c4)oc4cc(-c6ccncc6)c6ccccc6c45)c4ccccc34)ccc2c1 ; c1ccc(-c2ccc(-c3cc4oc5cc(-c6c7ccccc7c(-c7ccc8ccccc8c7)c7ccccc67)ccc5c4c4ccccc34)cc2)cc1 ; c1ccc2cc(-c3c4ccccc4c(-c4ccc5c(c4)oc4cc(-c6ccc7ccccc7c6)c6ccccc6c45)c4ccccc34)ccc2c1 ; c1ccc2cc(-c3c4ccccc4c(-c4ccc5c(c4)oc4cc(-c6ccc7oc8ccccc8c7c6)c6ccccc6c45)c4ccccc34)ccc2c1]

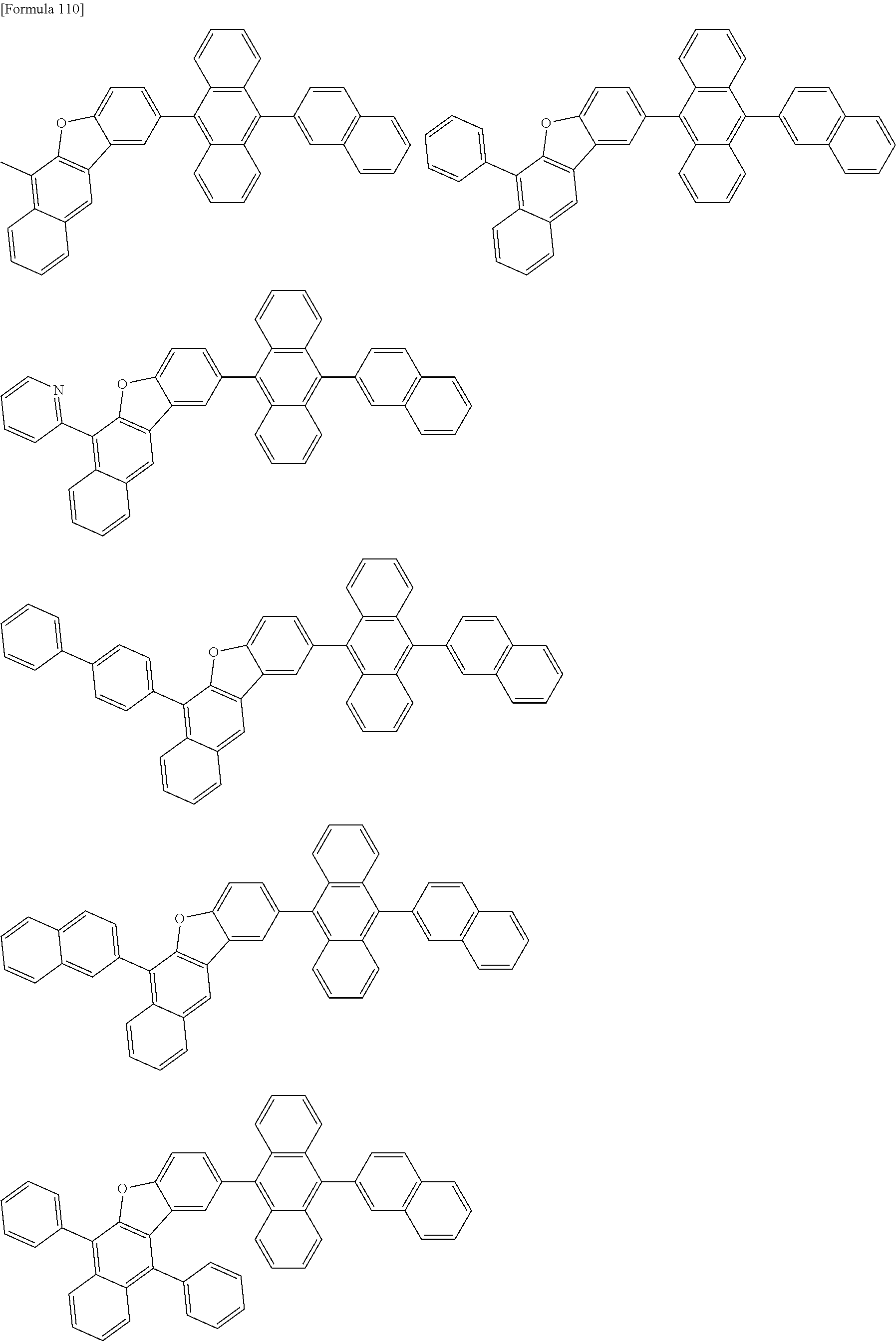

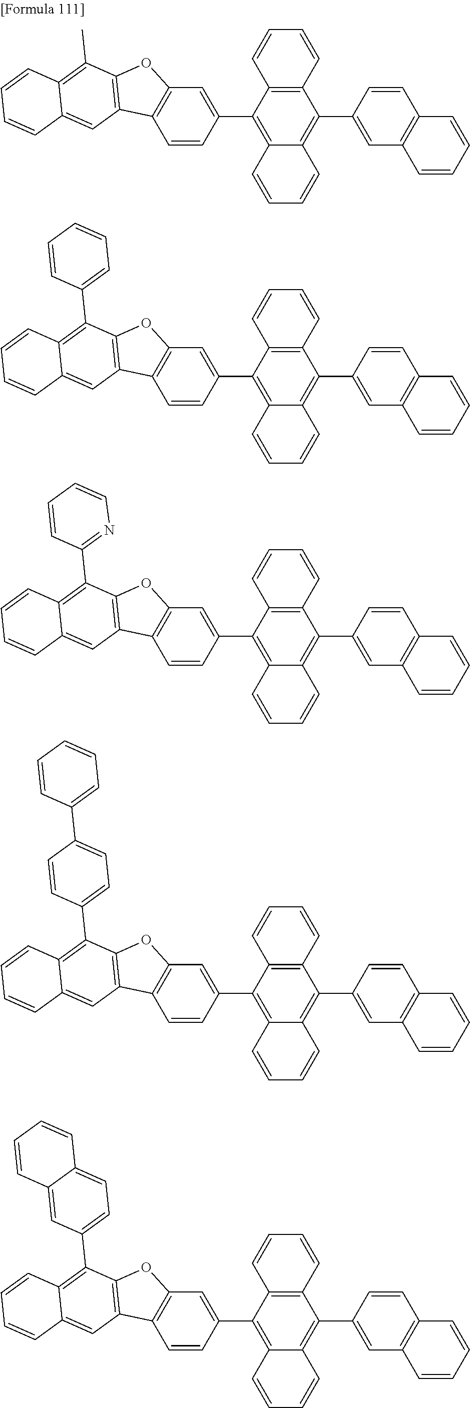 [SMILES: Cc1c2ccccc2cc2c1oc1cc(-c3c4ccccc4c(-c4ccc5ccccc5c4)c4ccccc34)ccc12 ; c1ccc(-c2c3ccccc3cc3c2oc2cc(-c4c5ccccc5c(-c5ccc6ccccc6c5)c5ccccc45)ccc23)cc1 ; c1ccc(-c2c3ccccc3cc3c2oc2cc(-c4c5ccccc5c(-c5ccc6ccccc6c5)c5ccccc45)ccc23)nc1 ; c1ccc(-c2ccc(-c3c4ccccc4cc4c3oc3cc(-c5c6ccccc6c(-c6ccc7ccccc7c6)c6ccccc56)ccc34)cc2)cc1 ; c1ccc2cc(-c3c4ccccc4c(-c4ccc5c(c4)oc4c(-c6ccc7ccccc7c6)c6ccccc6cc45)c4ccccc34)ccc2c1]

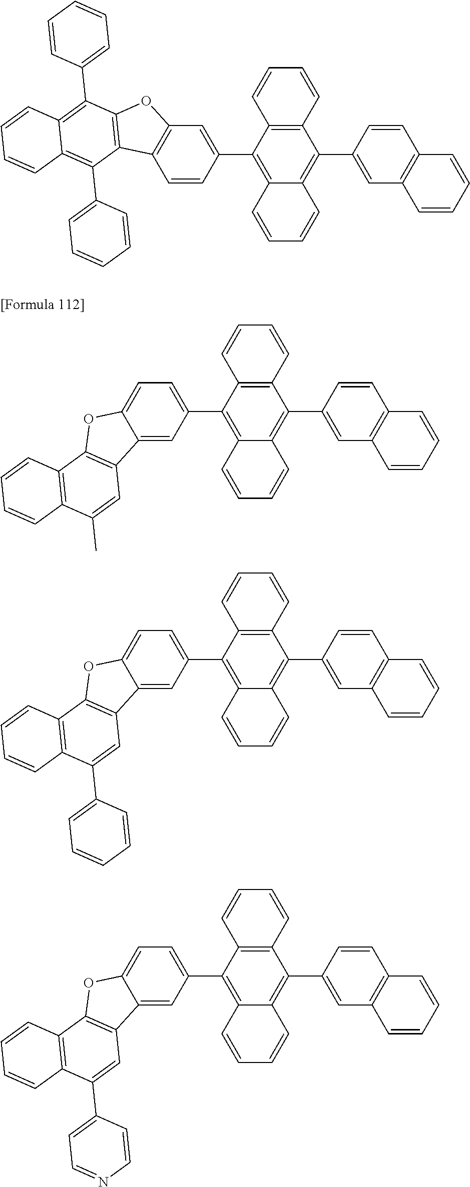 [SMILES: c1ccc(-c2c3ccccc3c(-c3ccccc3)c3c2oc2cc(-c4c5ccccc5c(-c5ccc6ccccc6c5)c5ccccc45)ccc23)cc1 ; Cc1cc2c3cc(-c4c5ccccc5c(-c5ccc6ccccc6c5)c5ccccc45)ccc3oc2c2ccccc12 ; c1ccc(-c2cc3c4cc(-c5c6ccccc6c(-c6ccc7ccccc7c6)c6ccccc56)ccc4oc3c3ccccc23)cc1 ; c1ccc2cc(-c3c4ccccc4c(-c4ccc5oc6c7ccccc7c(-c7ccncc7)cc6c5c4)c4ccccc34)ccc2c1]

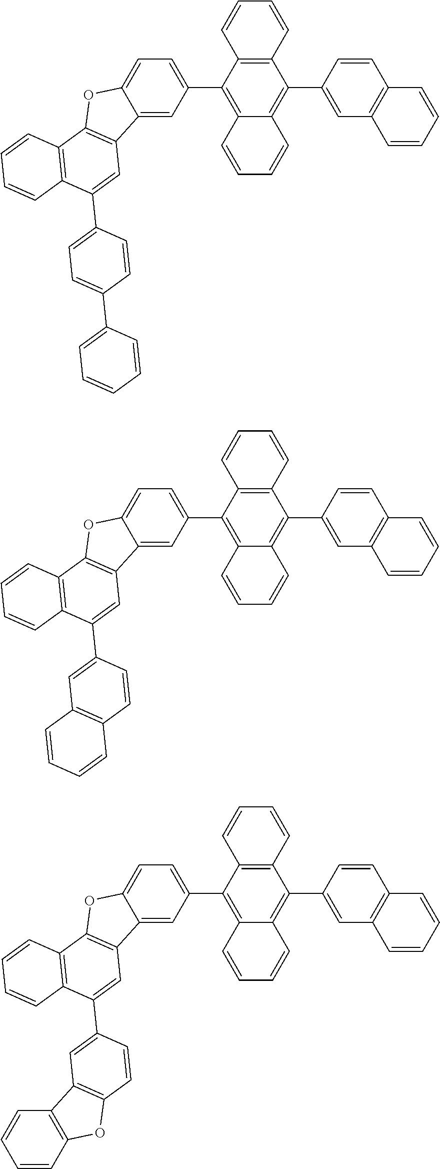 [SMILES: c1ccc(-c2ccc(-c3cc4c5cc(-c6c7ccccc7c(-c7ccc8ccccc8c7)c7ccccc67)ccc5oc4c4ccccc34)cc2)cc1 ; c1ccc2cc(-c3c4ccccc4c(-c4ccc5oc6c7ccccc7c(-c7ccc8ccccc8c7)cc6c5c4)c4ccccc34)ccc2c1 ; c1ccc2cc(-c3c4ccccc4c(-c4ccc5oc6c7ccccc7c(-c7ccc8oc9ccccc9c8c7)cc6c5c4)c4ccccc34)ccc2c1]

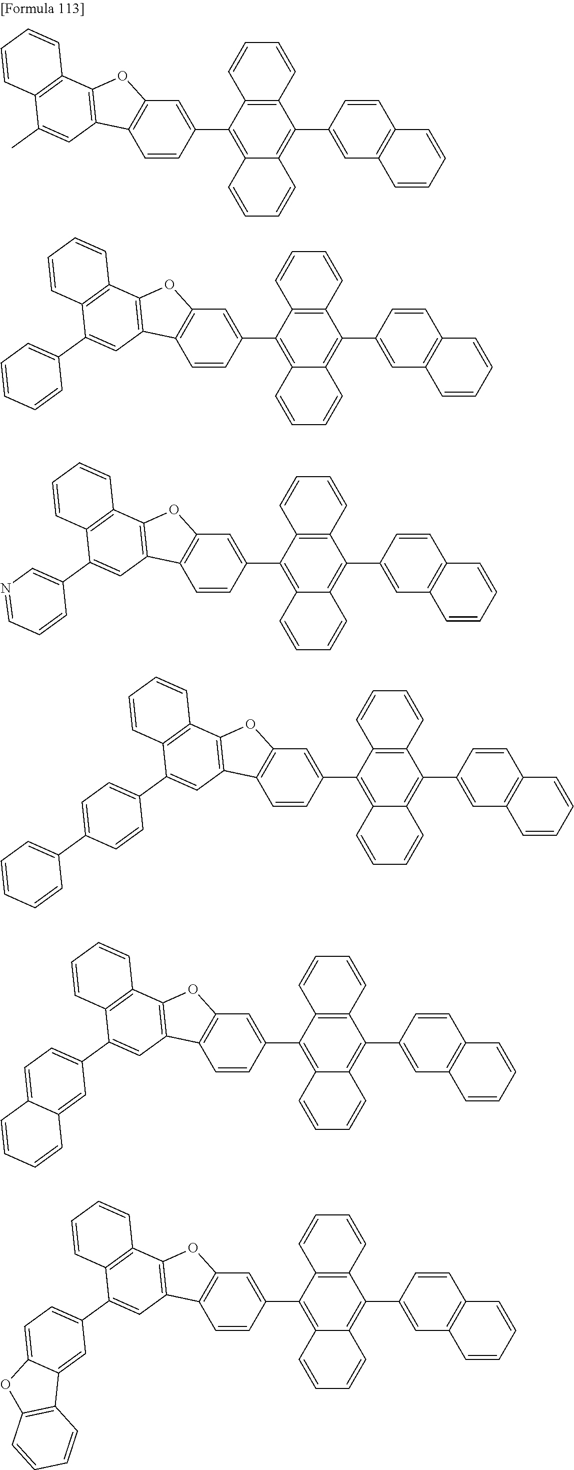

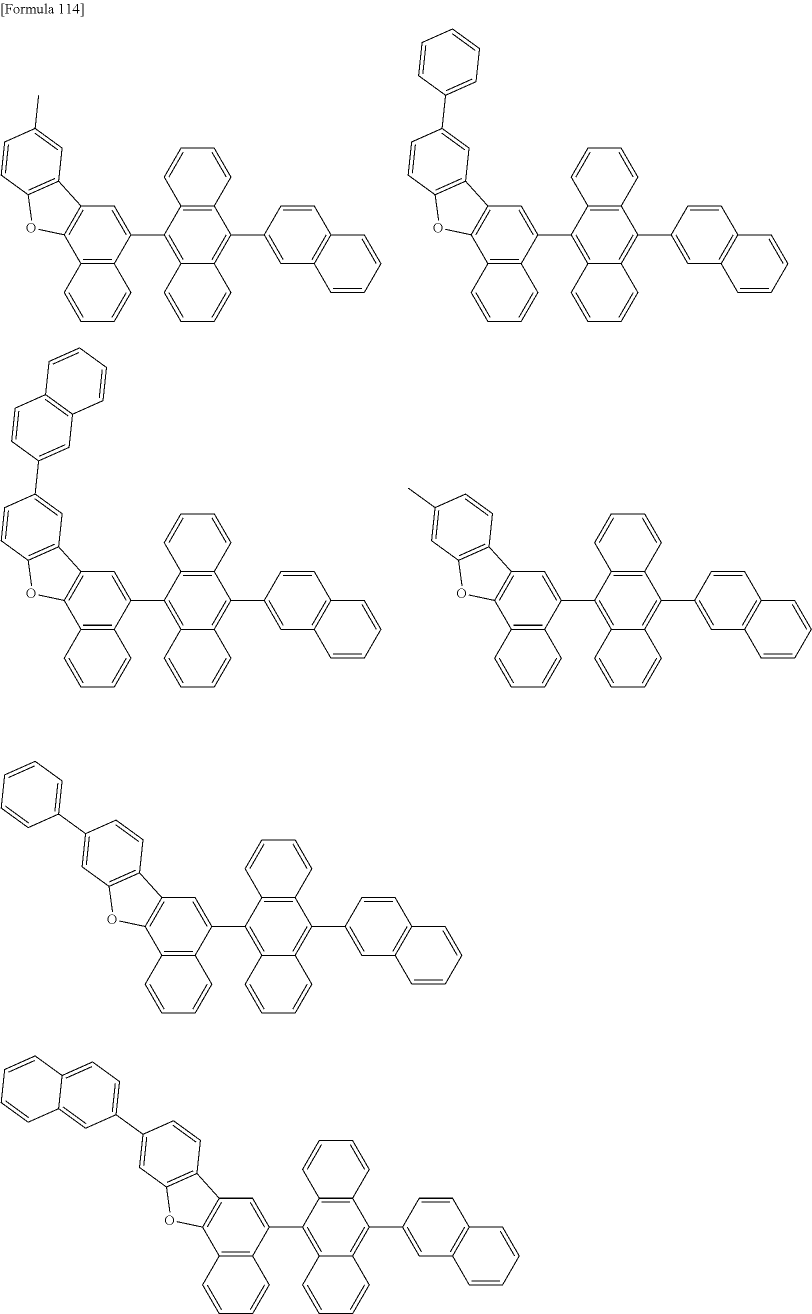

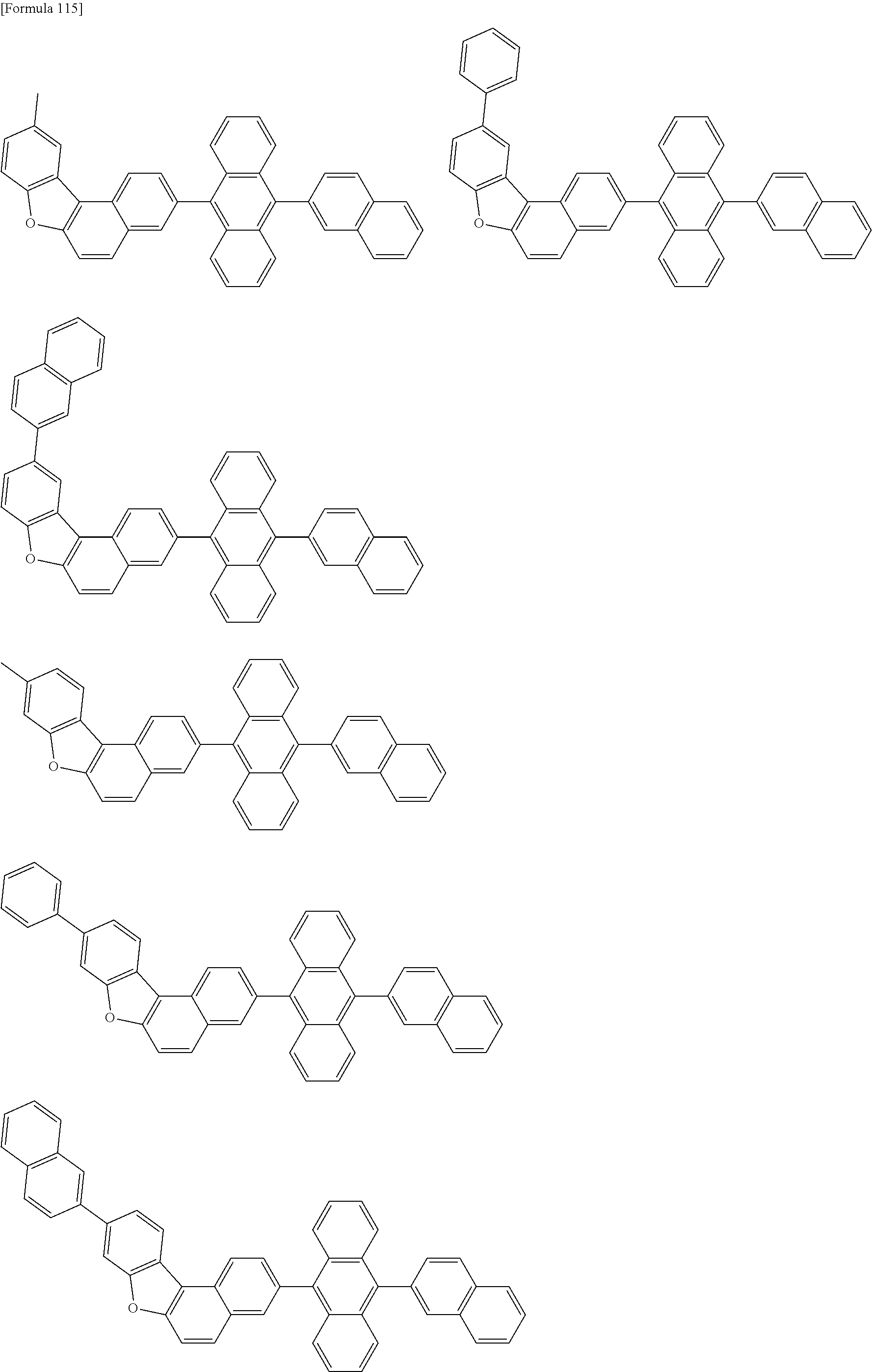

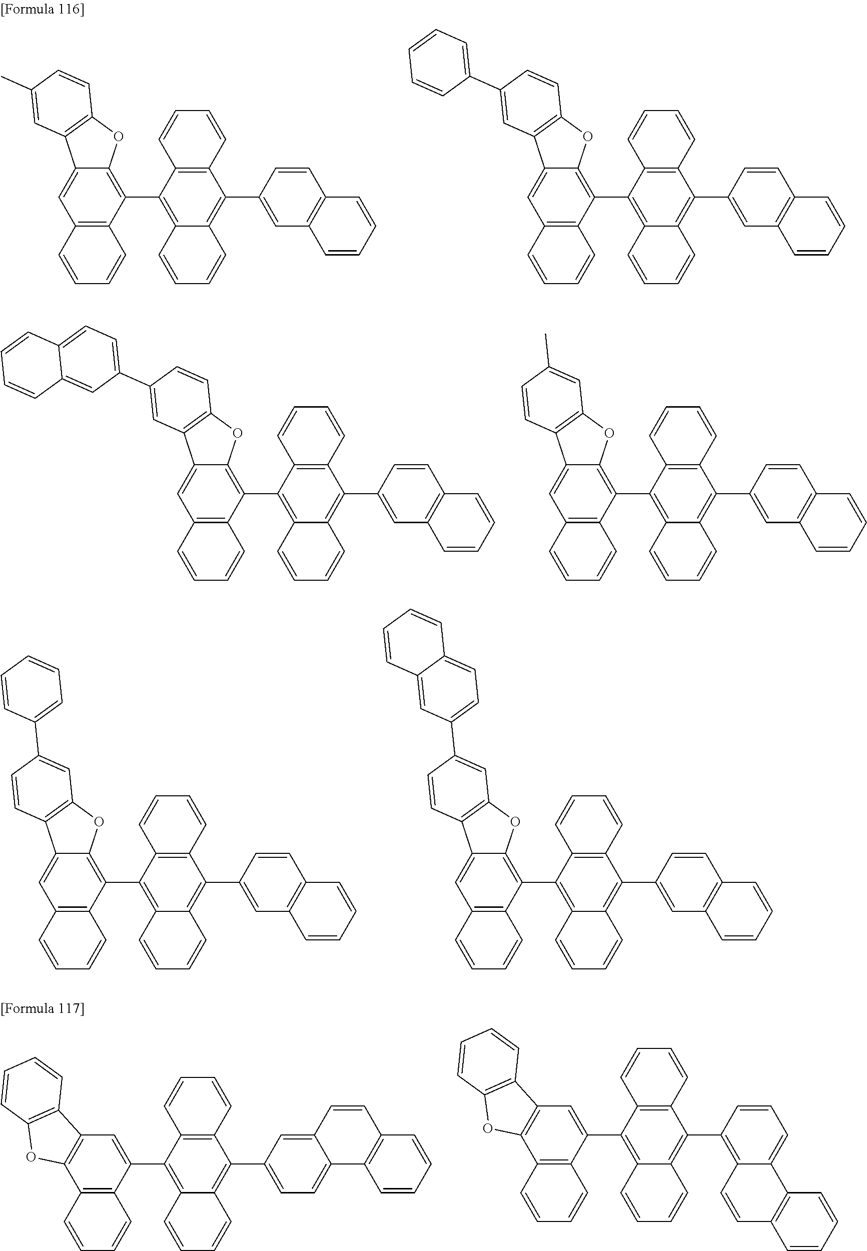

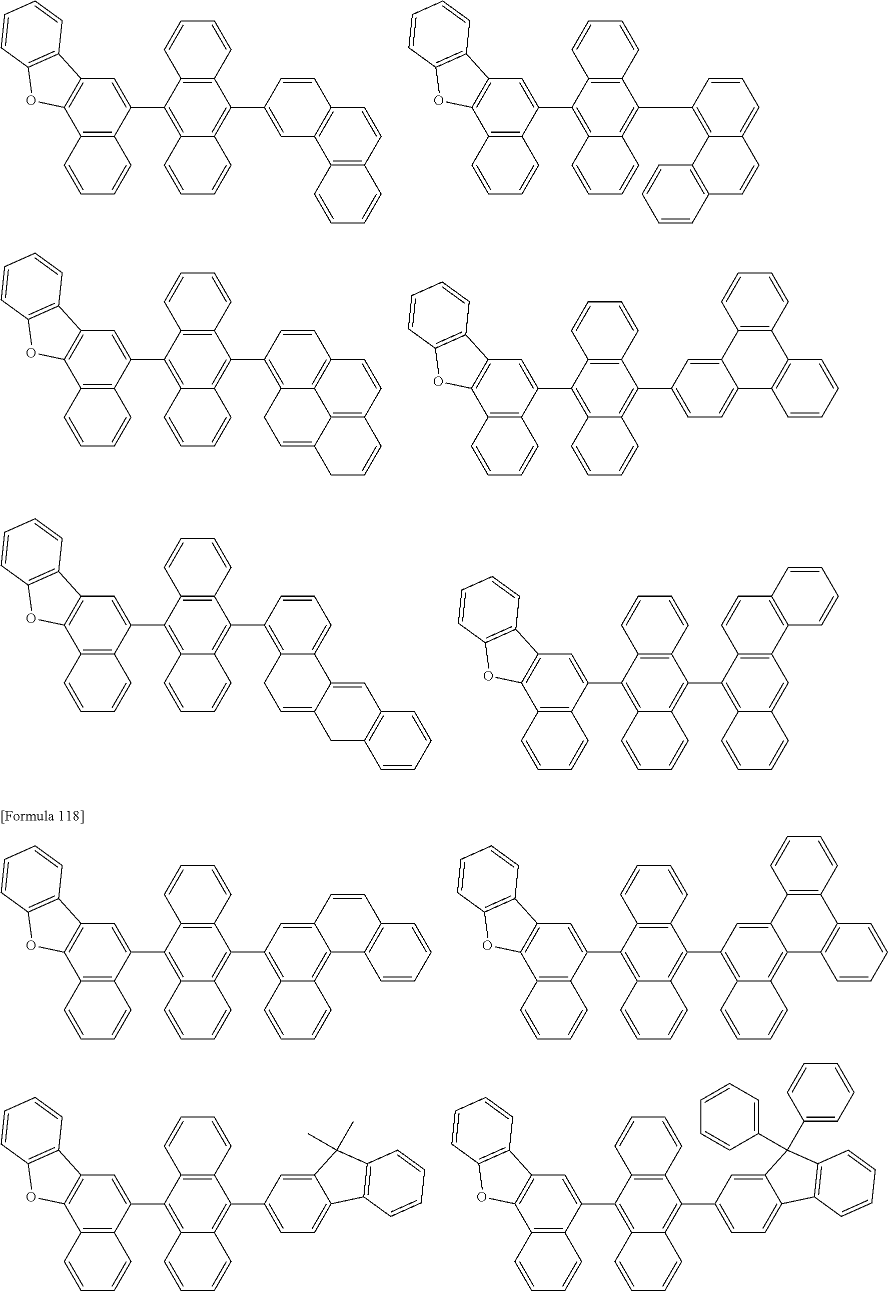

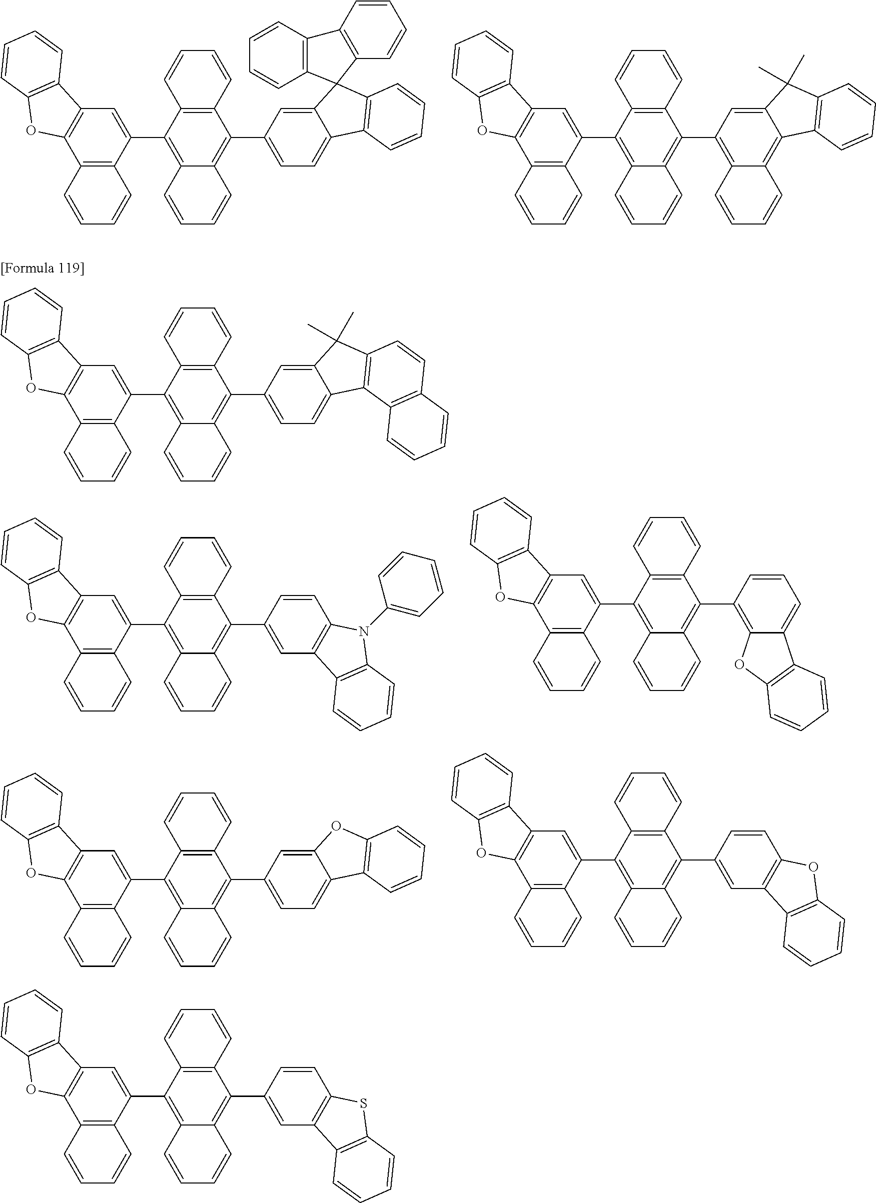

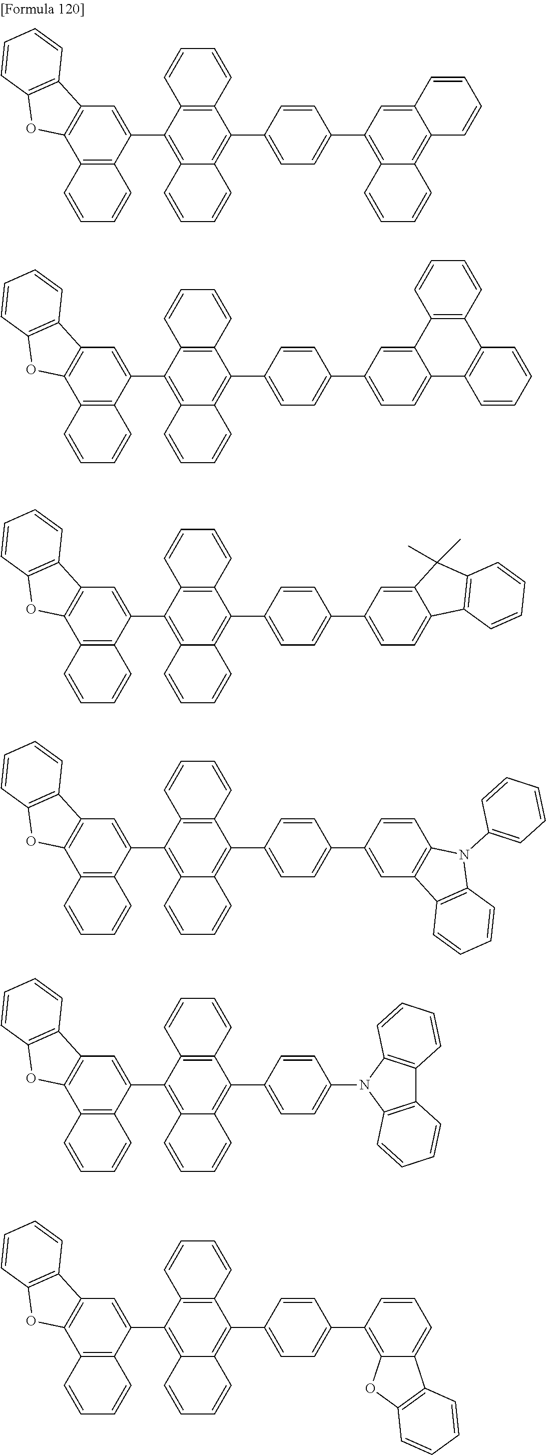

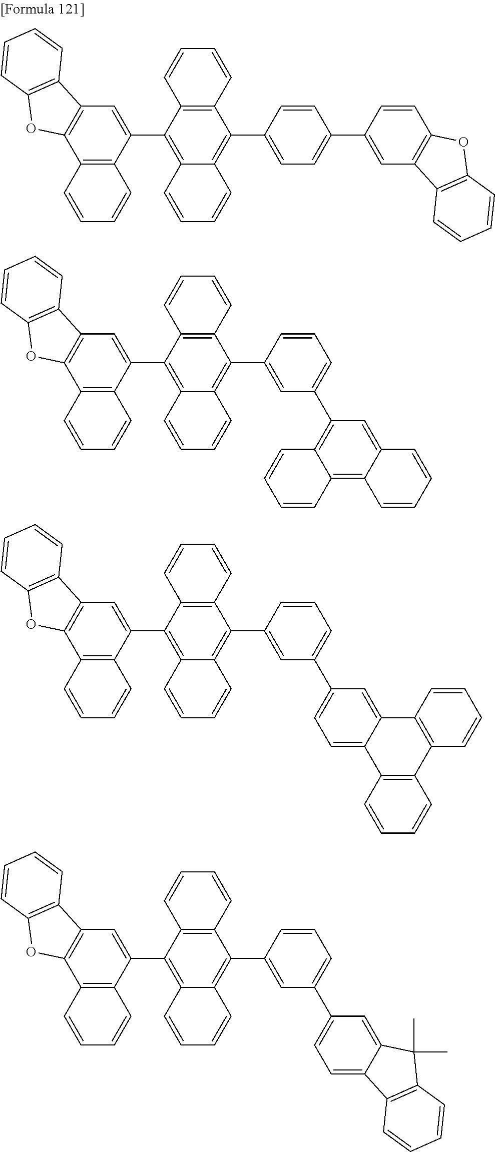 [SMILES: c1ccc2c(c1)oc1ccc(-c3ccc(-c4c5ccccc5c(-c5cc6c7ccccc7oc6c6ccccc56)c5ccccc45)cc3)cc12 ; c1cc(-c2c3ccccc3c(-c3cc4c5ccccc5oc4c4ccccc34)c3ccccc23)cc(-c2cc3ccccc3c3ccccc23)c1 ; c1cc(-c2ccc3c4ccccc4c4ccccc4c3c2)cc(-c2c3ccccc3c(-c3cc4c5ccccc5oc4c4ccccc34)c3ccccc23)c1 ; CC1(C)c2ccccc2-c2ccc(-c3cccc(-c4c5ccccc5c(-c5cc6c7ccccc7oc6c6ccccc56)c5ccccc45)c3)cc21]

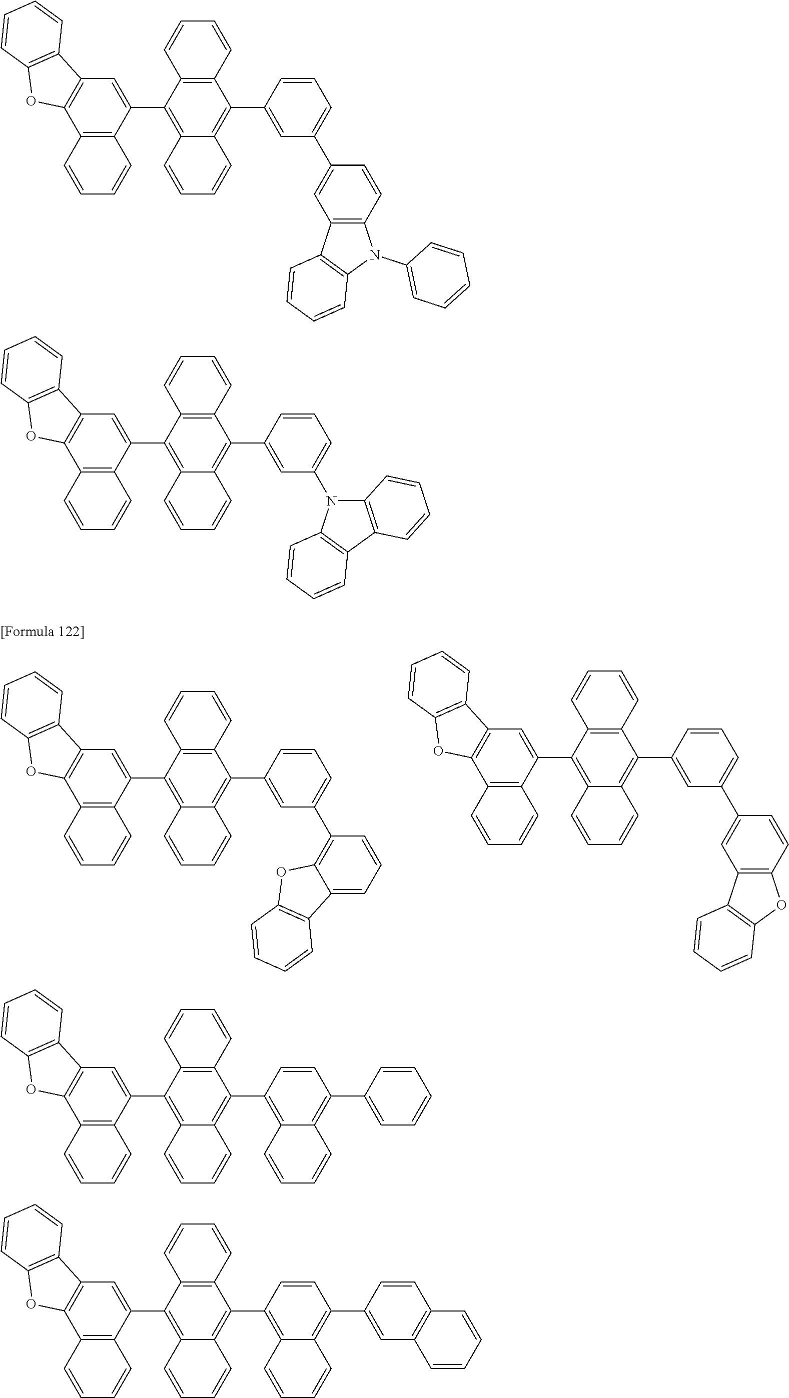

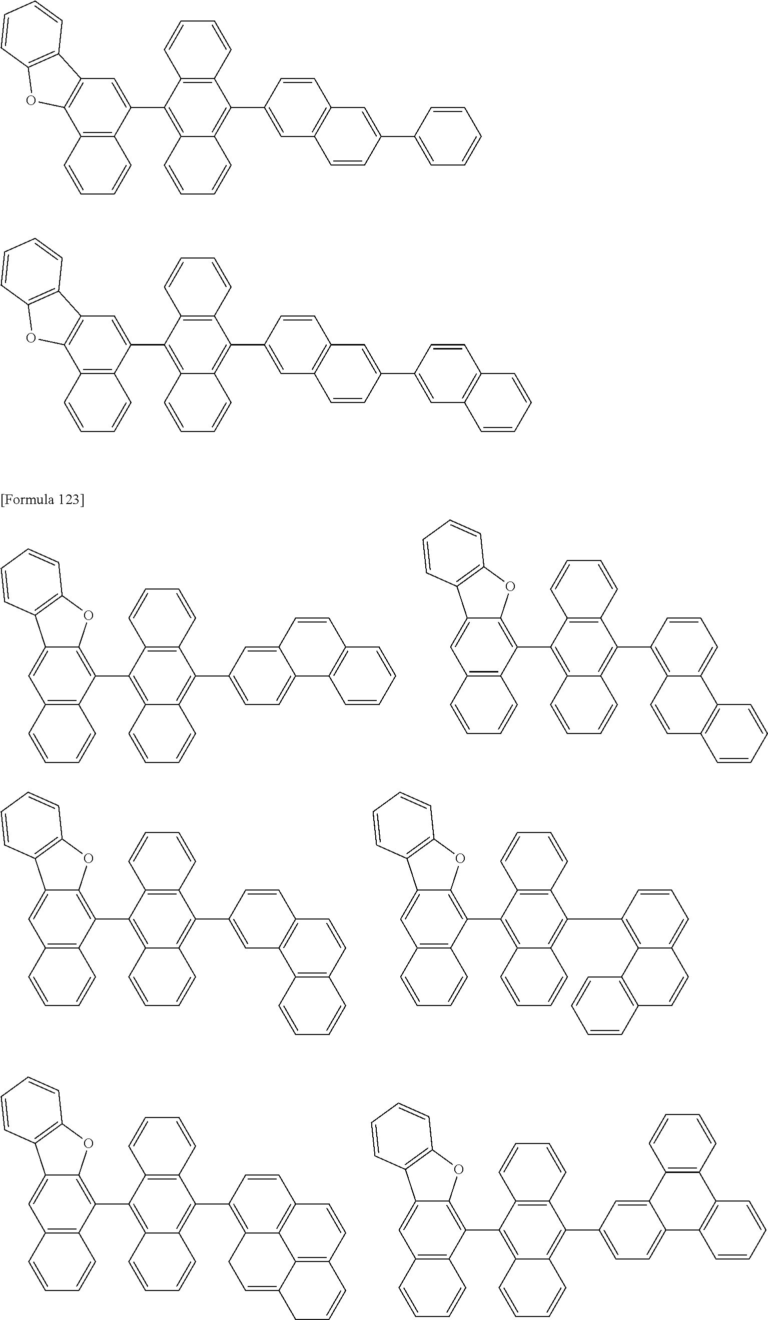

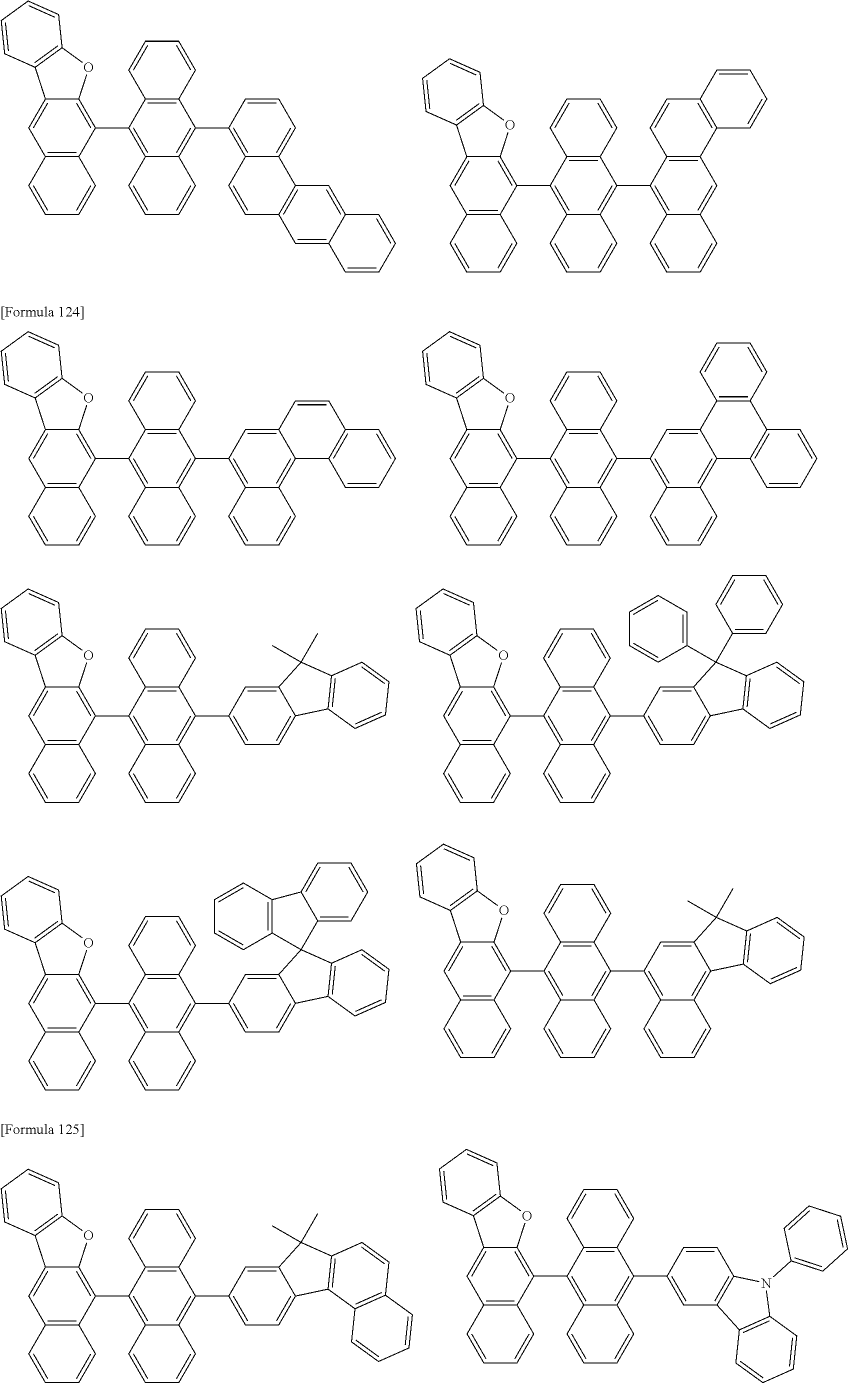

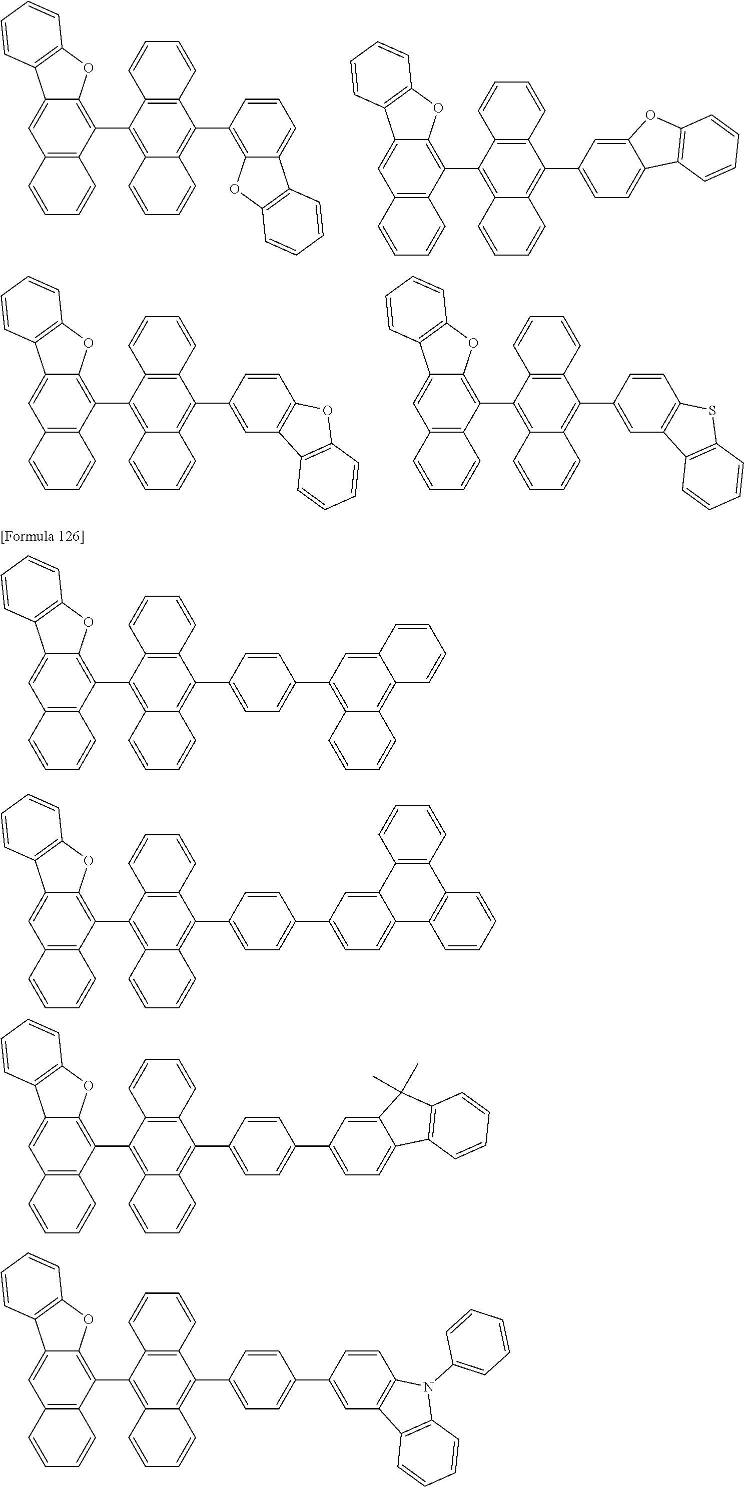

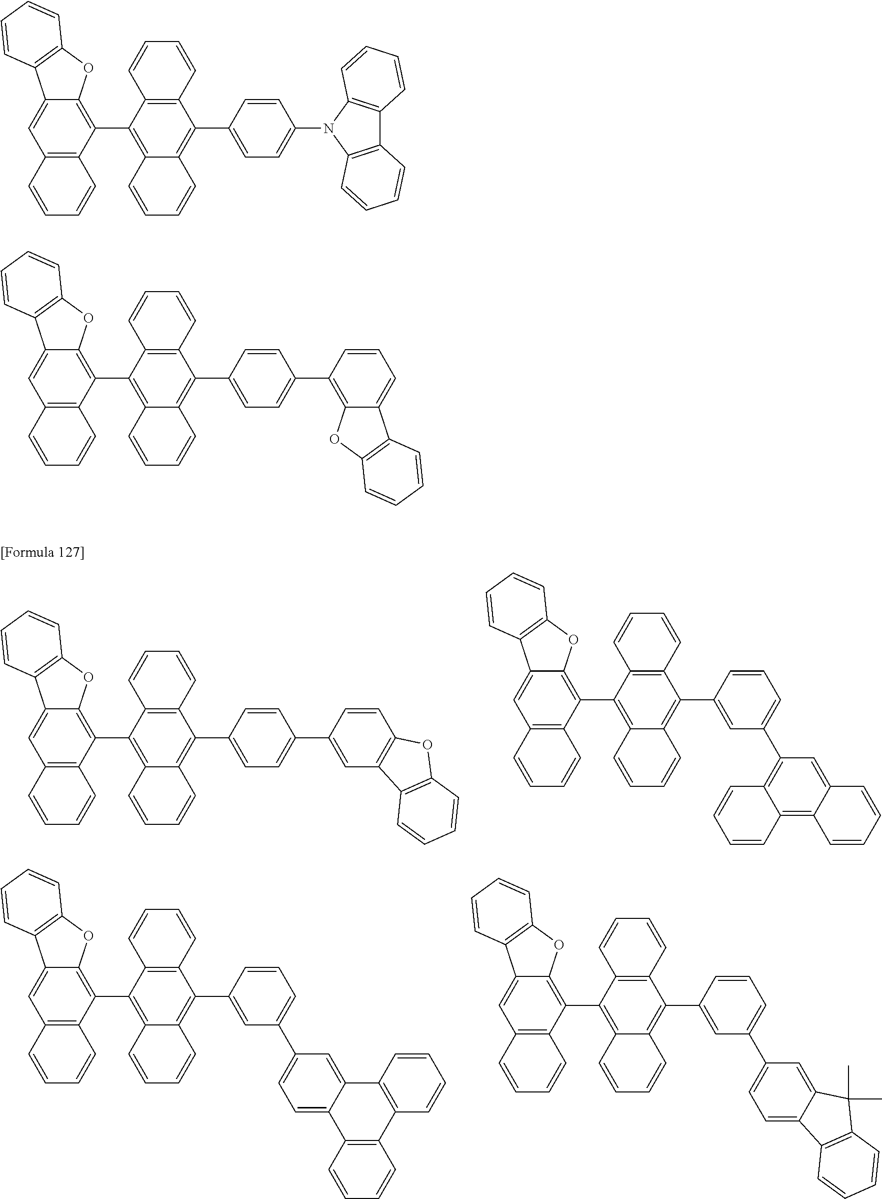

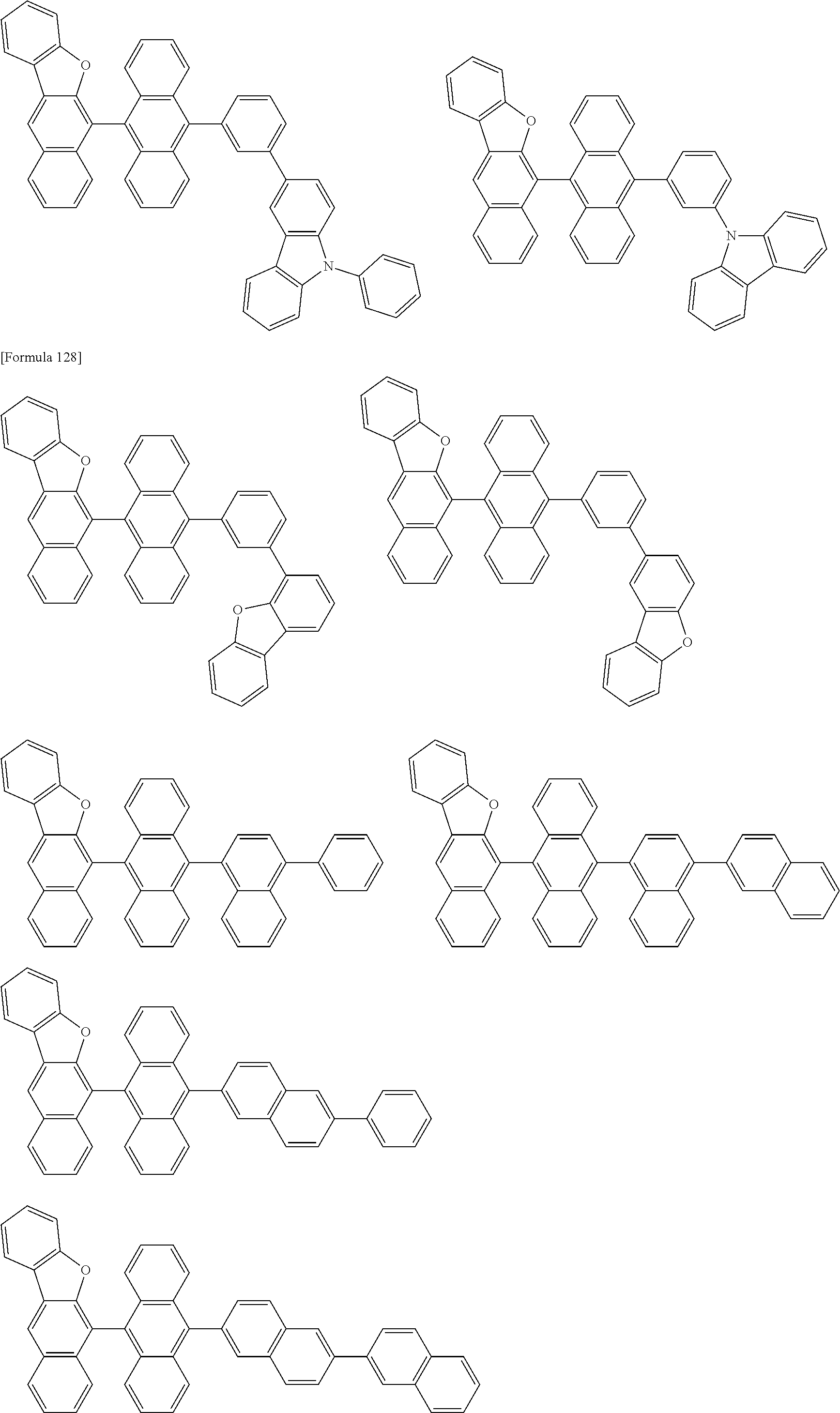

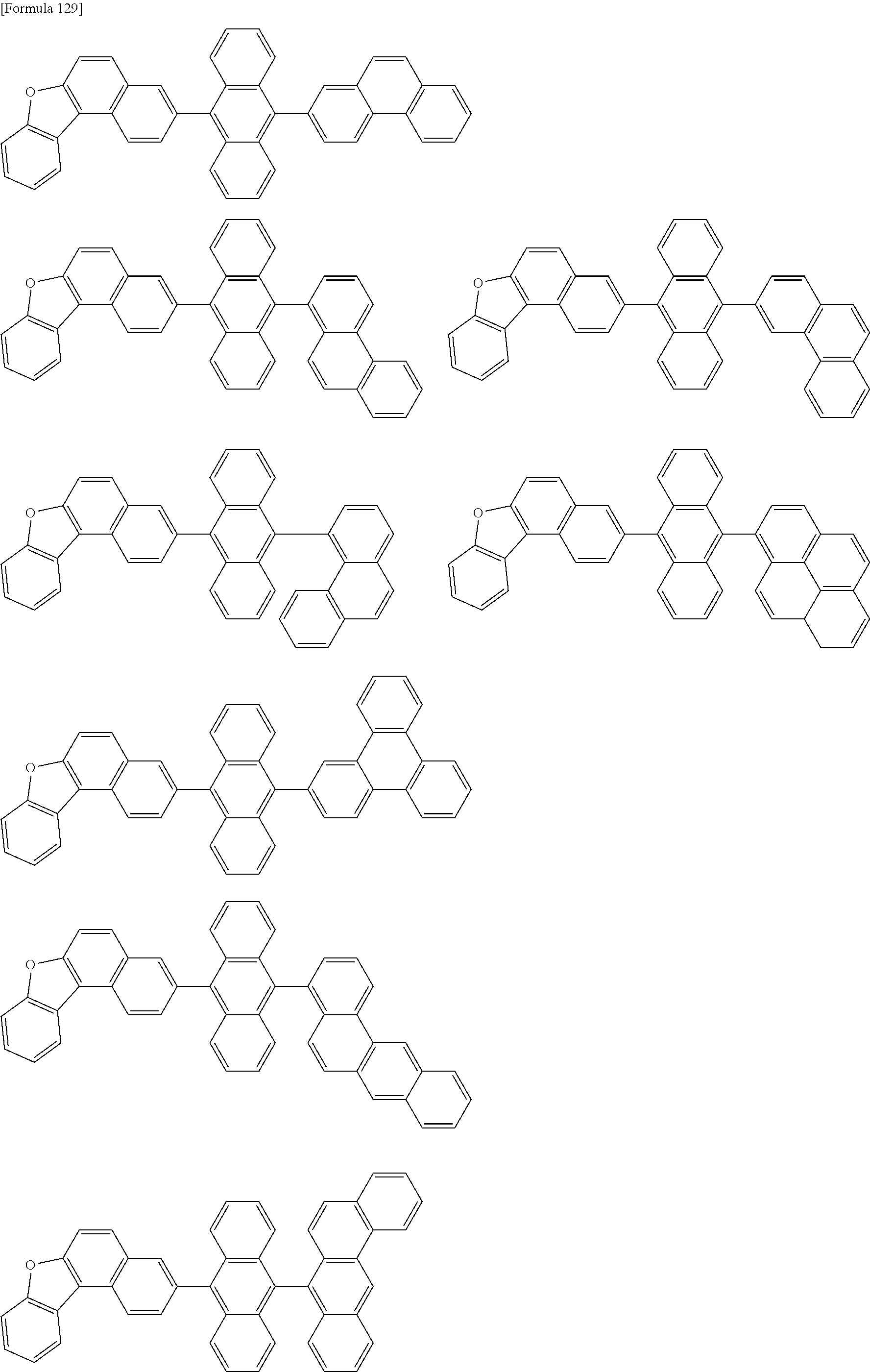

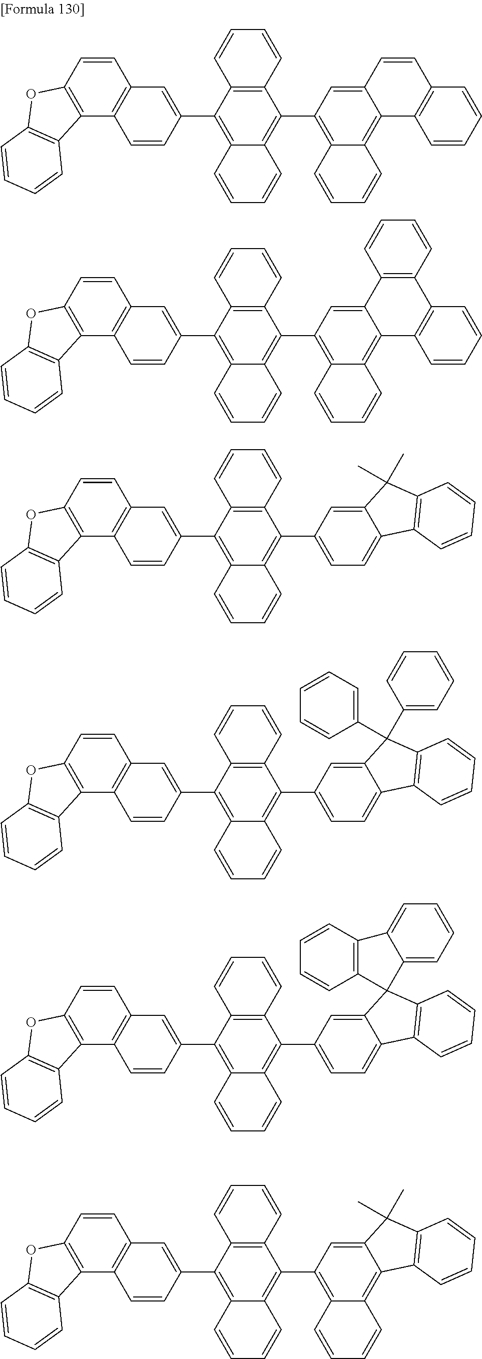

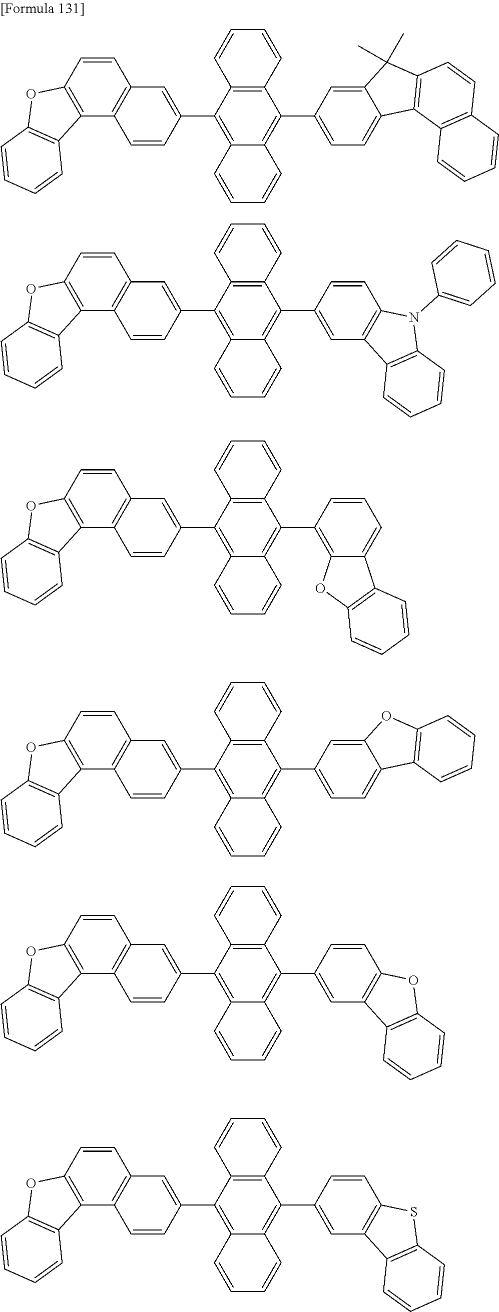

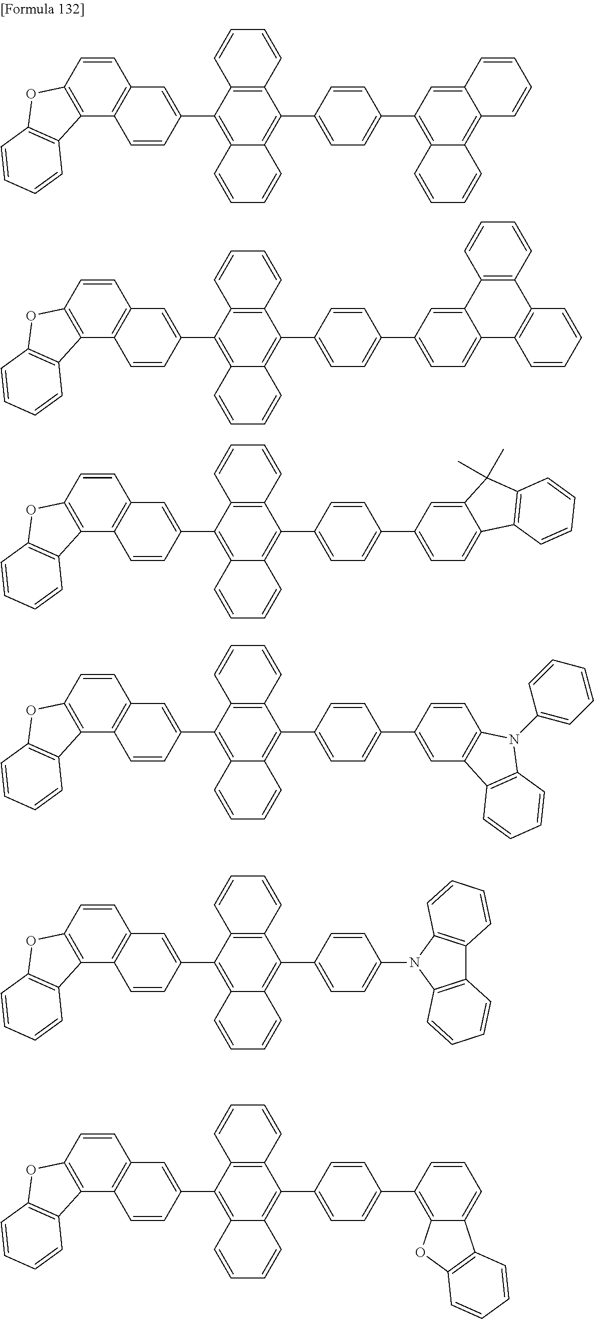

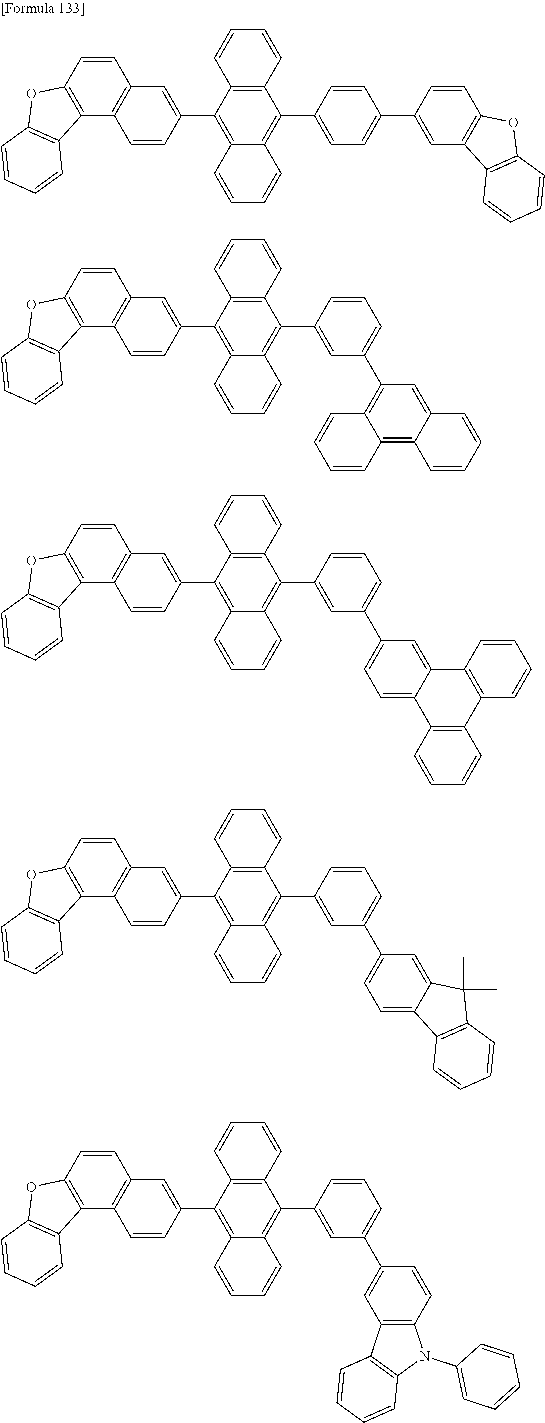

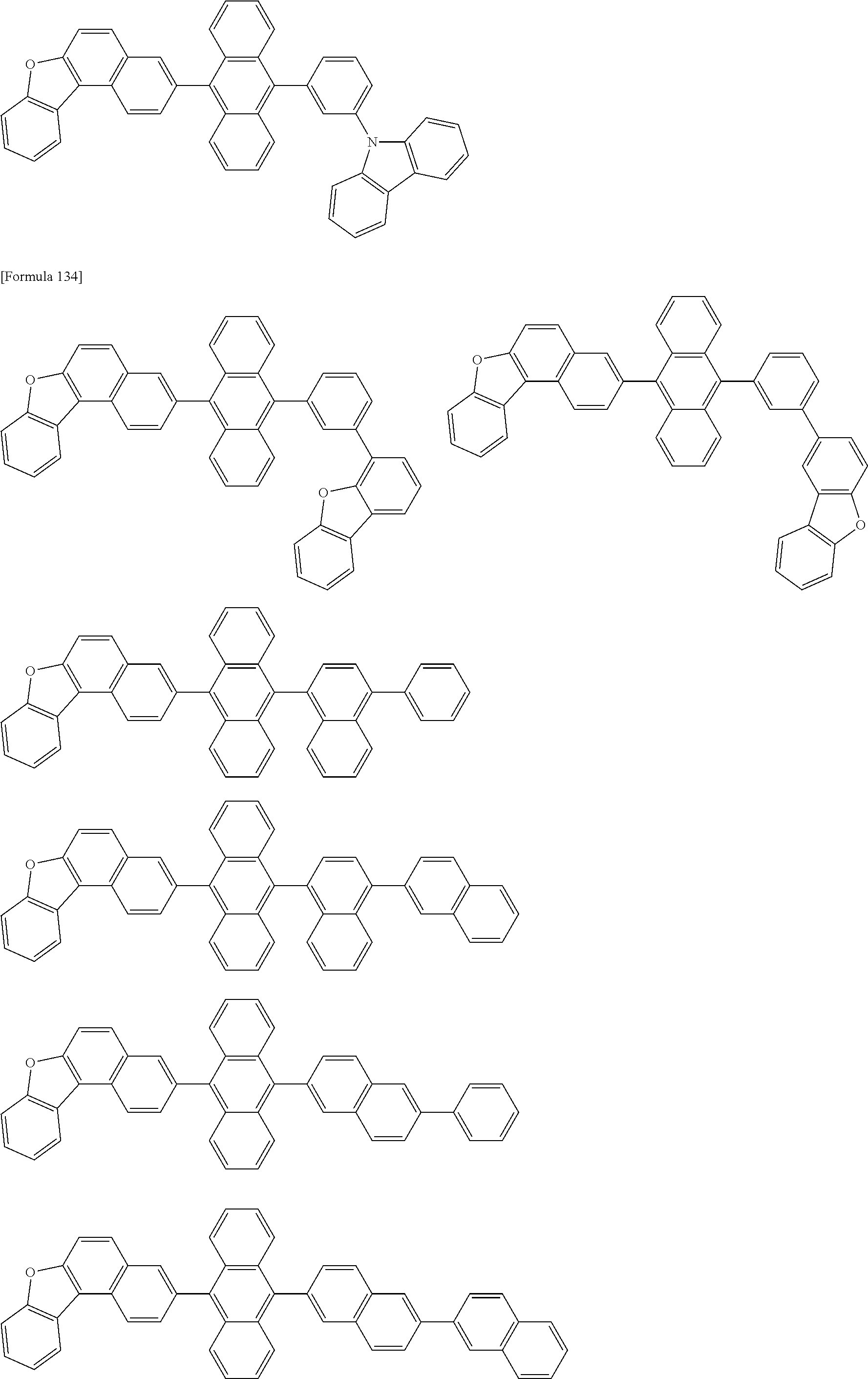

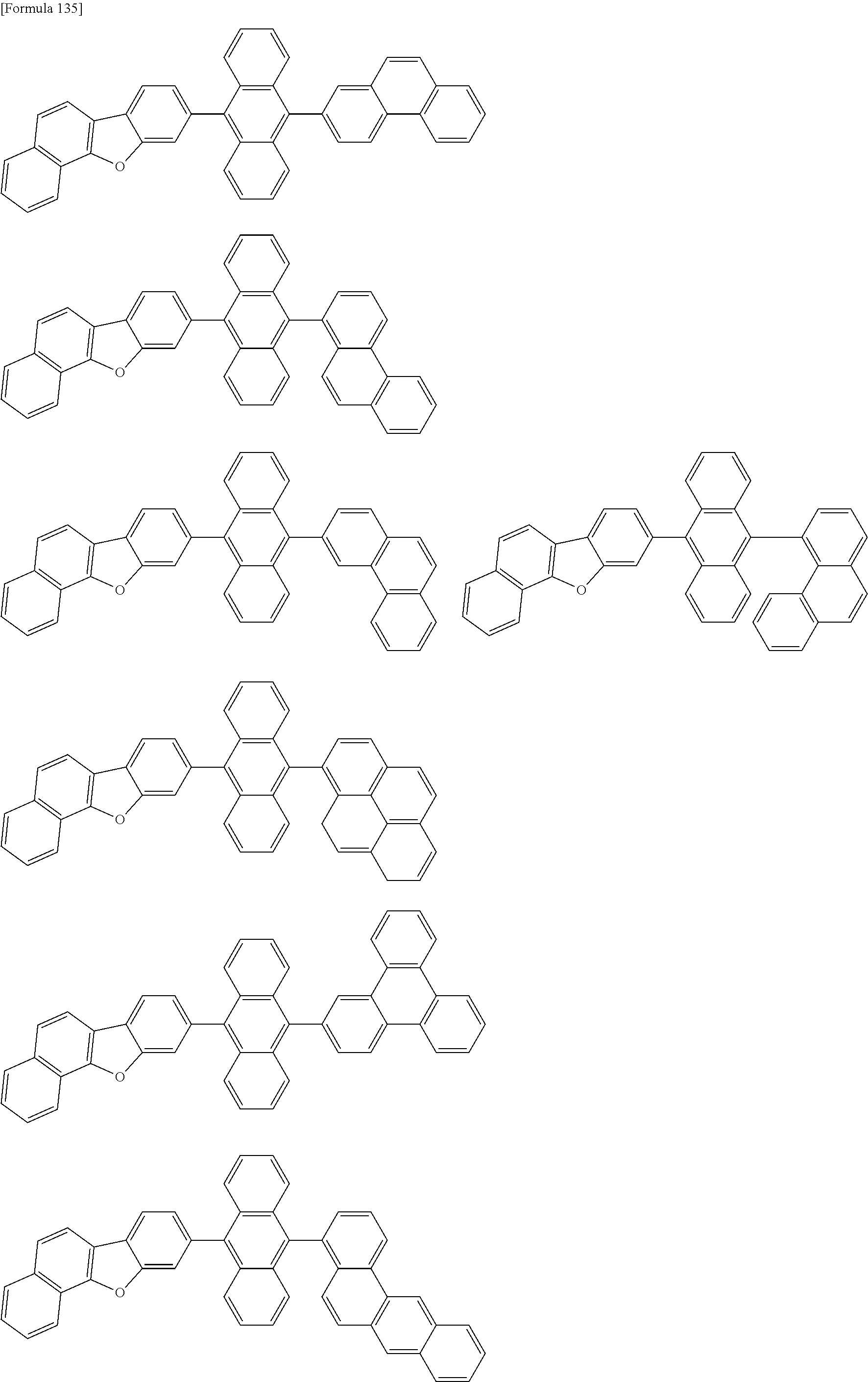

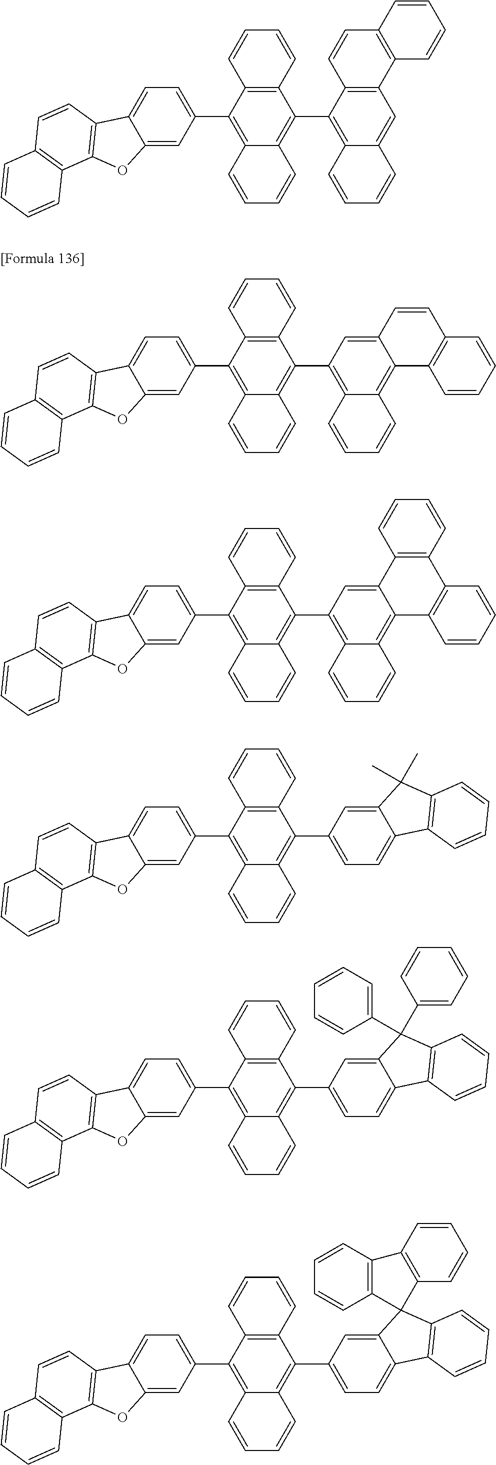

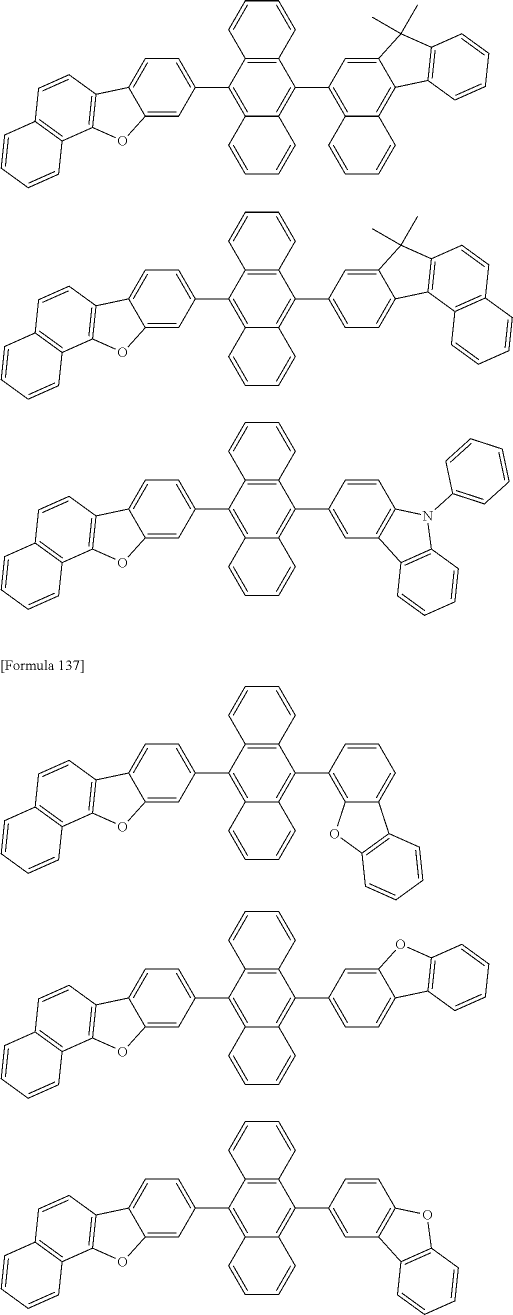

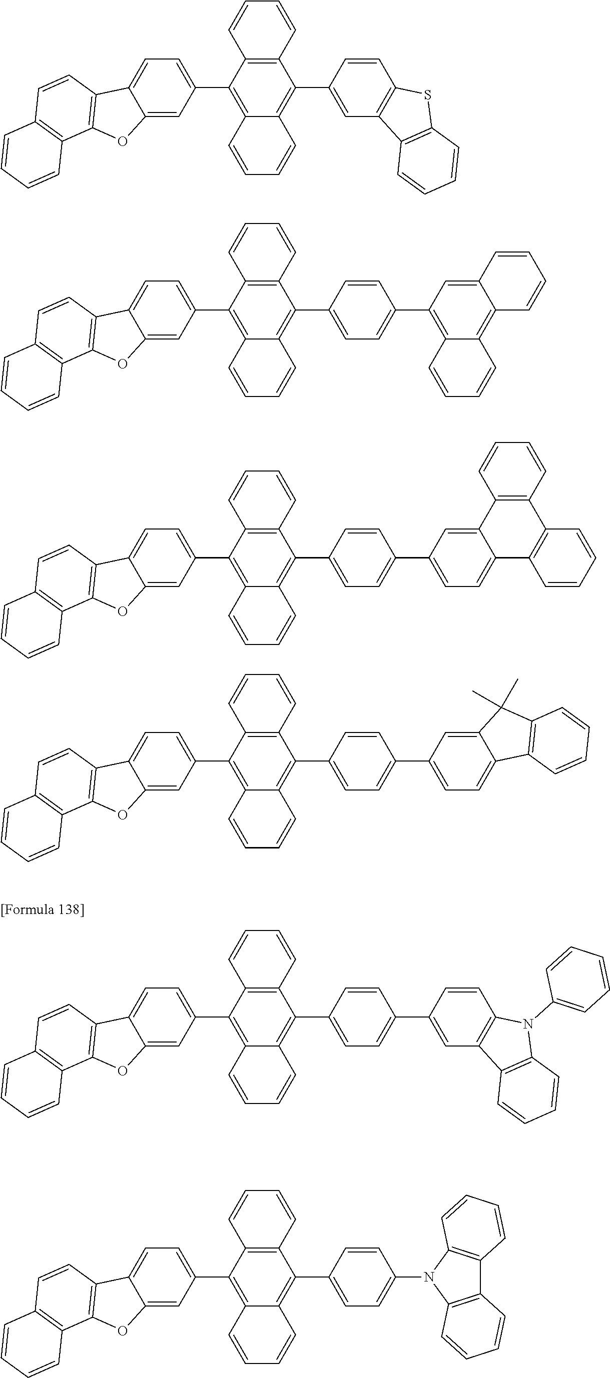

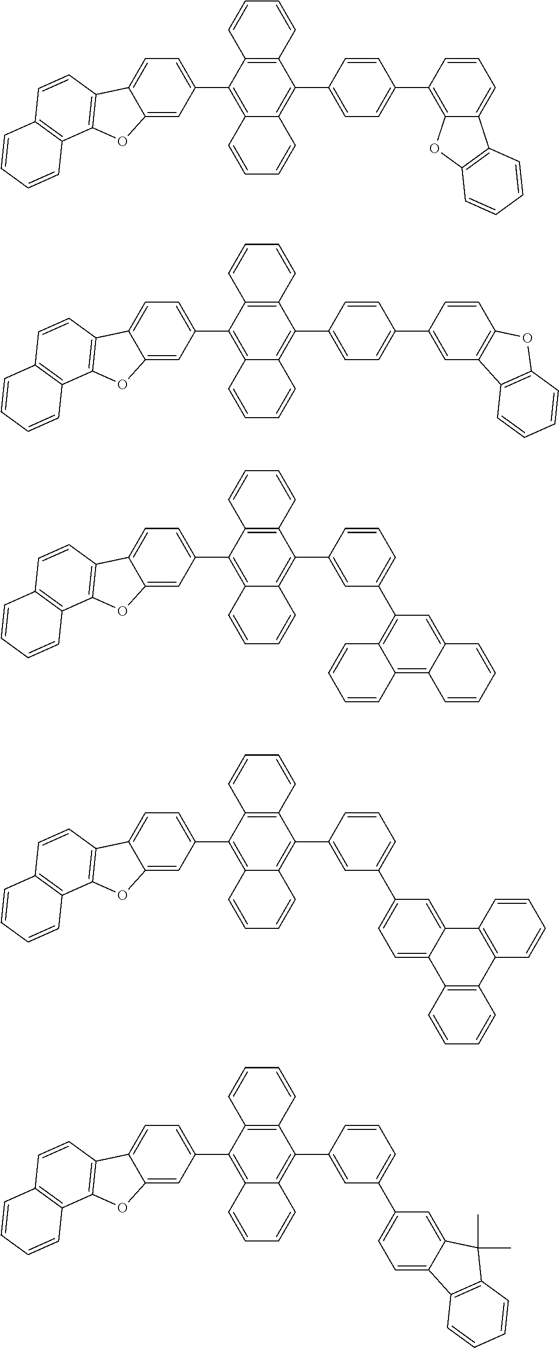 [SMILES: c1ccc2c(c1)ccc1c3ccc(-c4c5ccccc5c(-c5ccc(-c6cccc7c6oc6ccccc67)cc5)c5ccccc45)cc3oc21 ; c1ccc2c(c1)ccc1c3ccc(-c4c5ccccc5c(-c5ccc(-c6ccc7oc8ccccc8c7c6)cc5)c5ccccc45)cc3oc21 ; c1cc(-c2c3ccccc3c(-c3ccc4c(c3)oc3c5ccccc5ccc43)c3ccccc23)cc(-c2cc3ccccc3c3ccccc23)c1 ; c1cc(-c2ccc3c4ccccc4c4ccccc4c3c2)cc(-c2c3ccccc3c(-c3ccc4c(c3)oc3c5ccccc5ccc43)c3ccccc23)c1 ; CC1(C)c2ccccc2-c2ccc(-c3cccc(-c4c5ccccc5c(-c5ccc6c(c5)oc5c7ccccc7ccc65)c5ccccc45)c3)cc21]

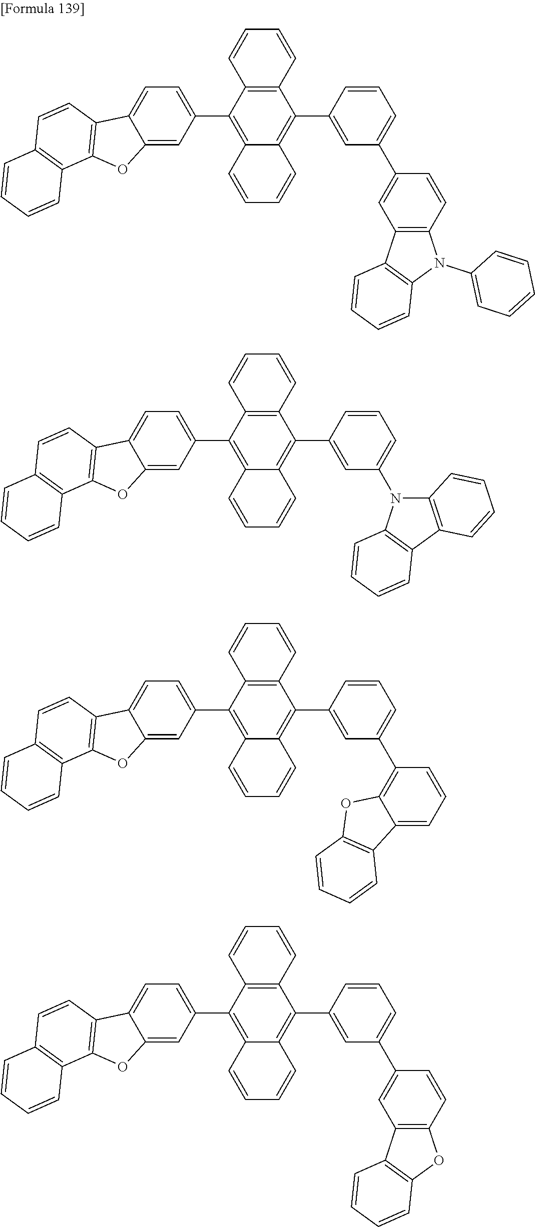 [SMILES: c1ccc(-n2c3ccccc3c3cc(-c4cccc(-c5c6ccccc6c(-c6ccc7c(c6)oc6c8ccccc8ccc76)c6ccccc56)c4)ccc32)cc1 ; c1cc(-c2c3ccccc3c(-c3ccc4c(c3)oc3c5ccccc5ccc43)c3ccccc23)cc(-n2c3ccccc3c3ccccc32)c1 ; c1cc(-c2c3ccccc3c(-c3ccc4c(c3)oc3c5ccccc5ccc43)c3ccccc23)cc(-c2cccc3c2oc2ccccc23)c1 ; c1cc(-c2ccc3oc4ccccc4c3c2)cc(-c2c3ccccc3c(-c3ccc4c(c3)oc3c5ccccc5ccc43)c3ccccc23)c1]

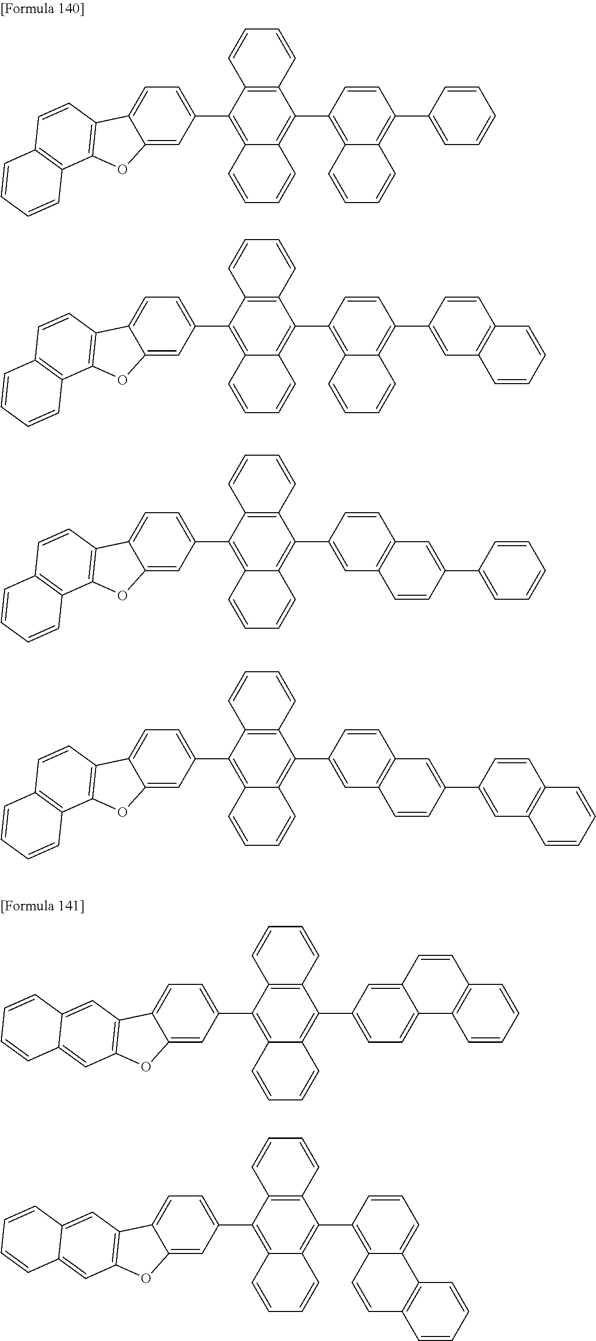

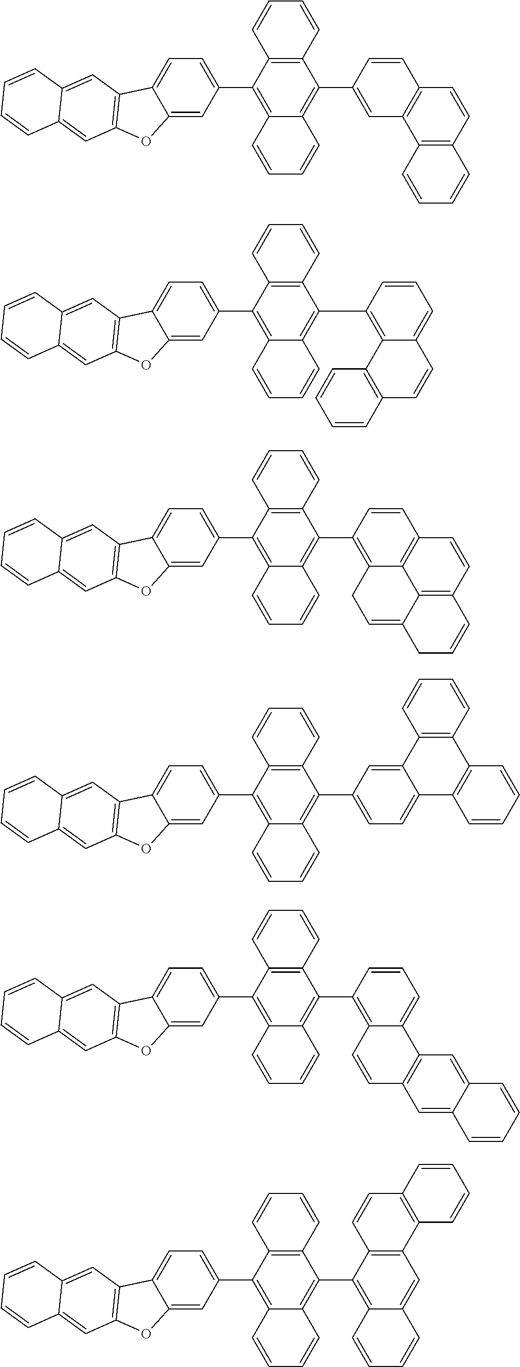

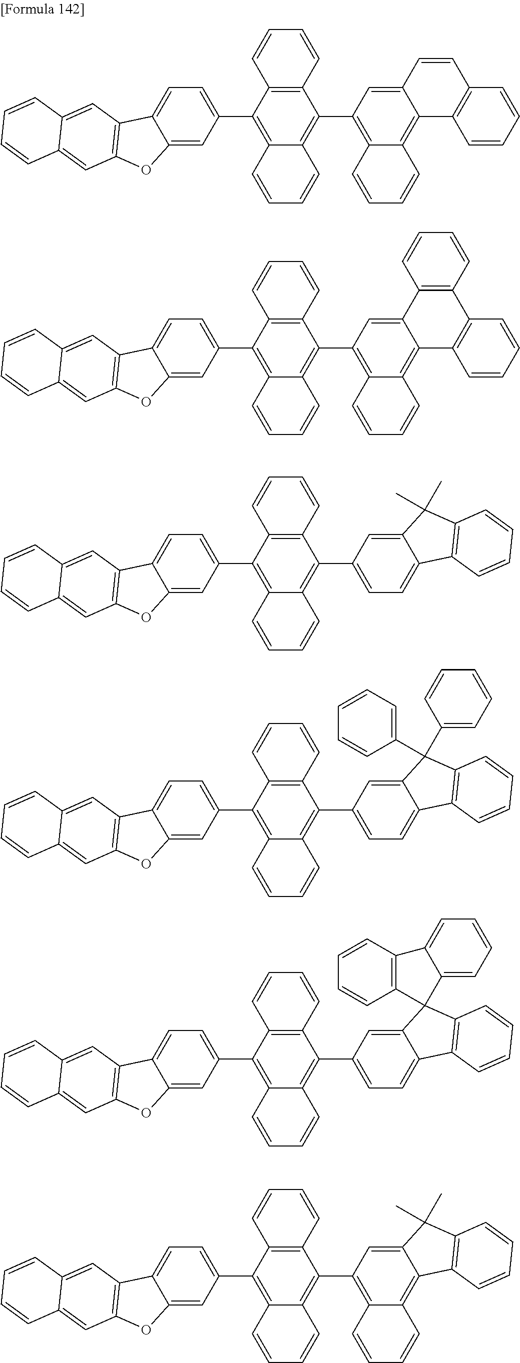

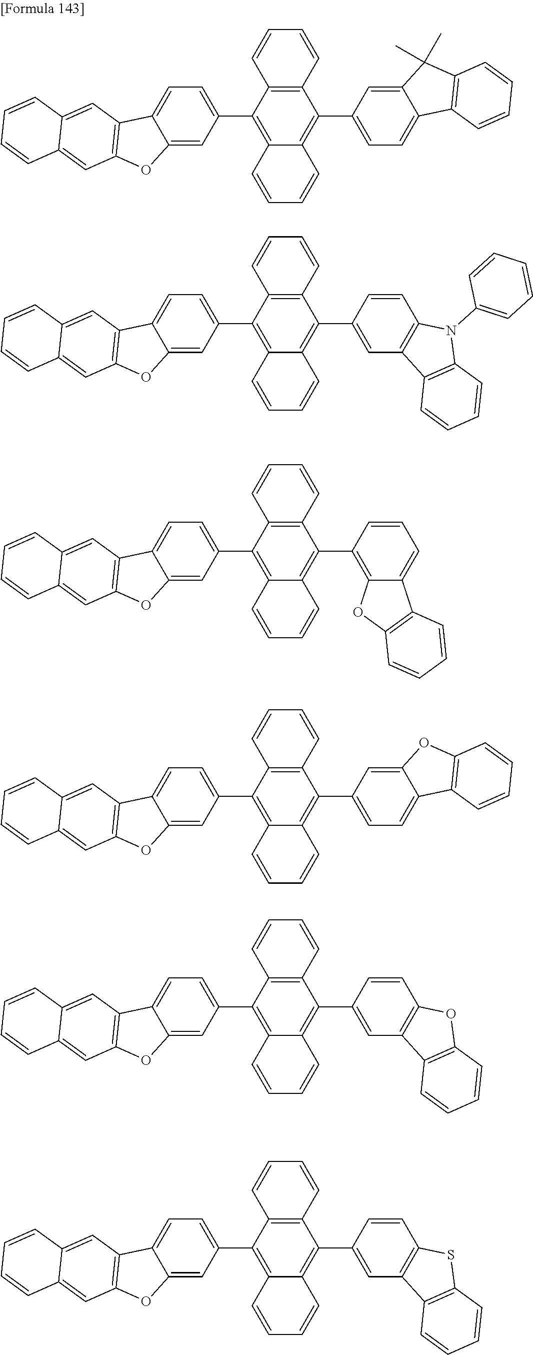

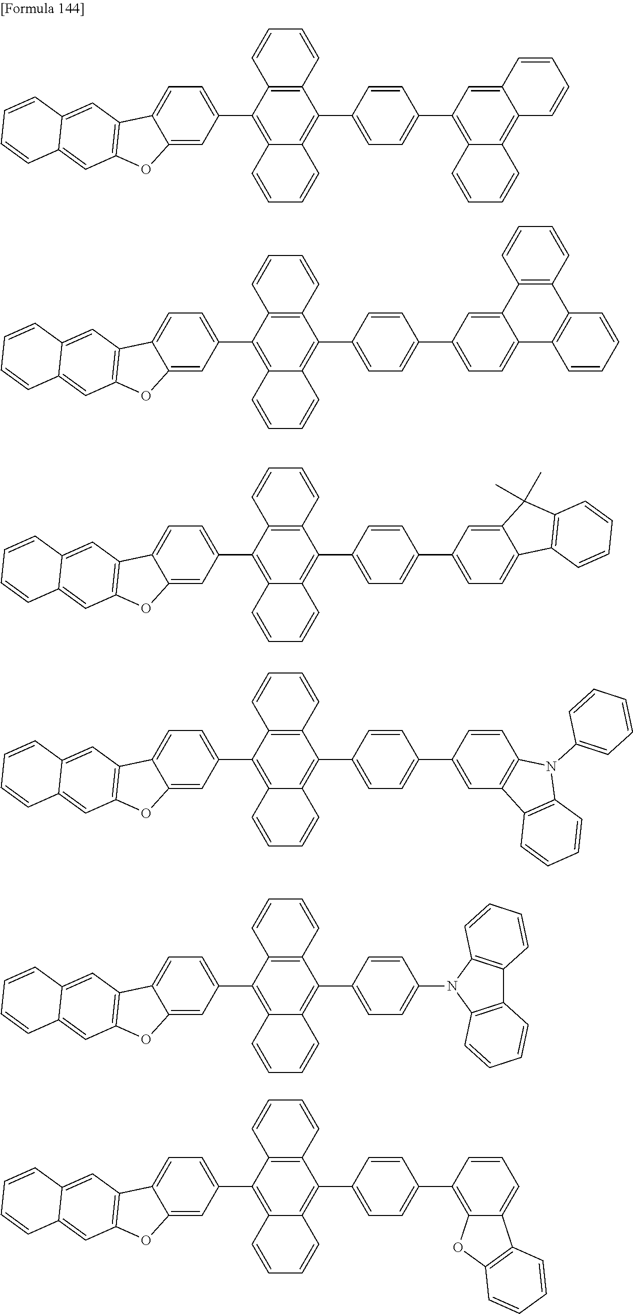

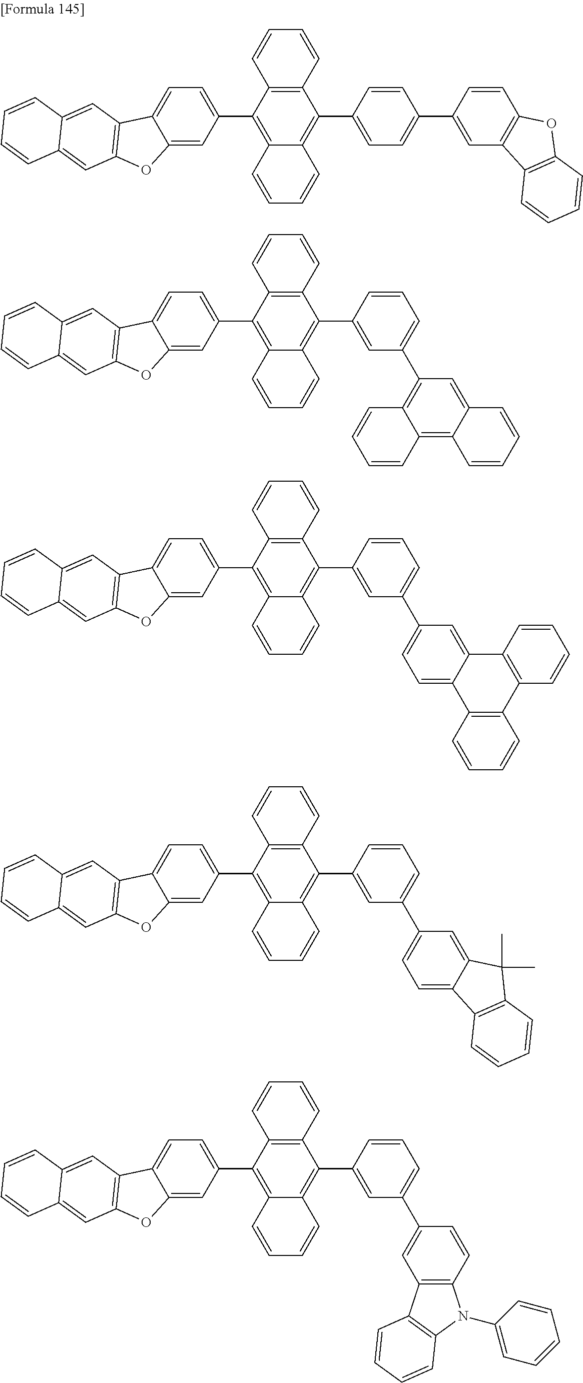

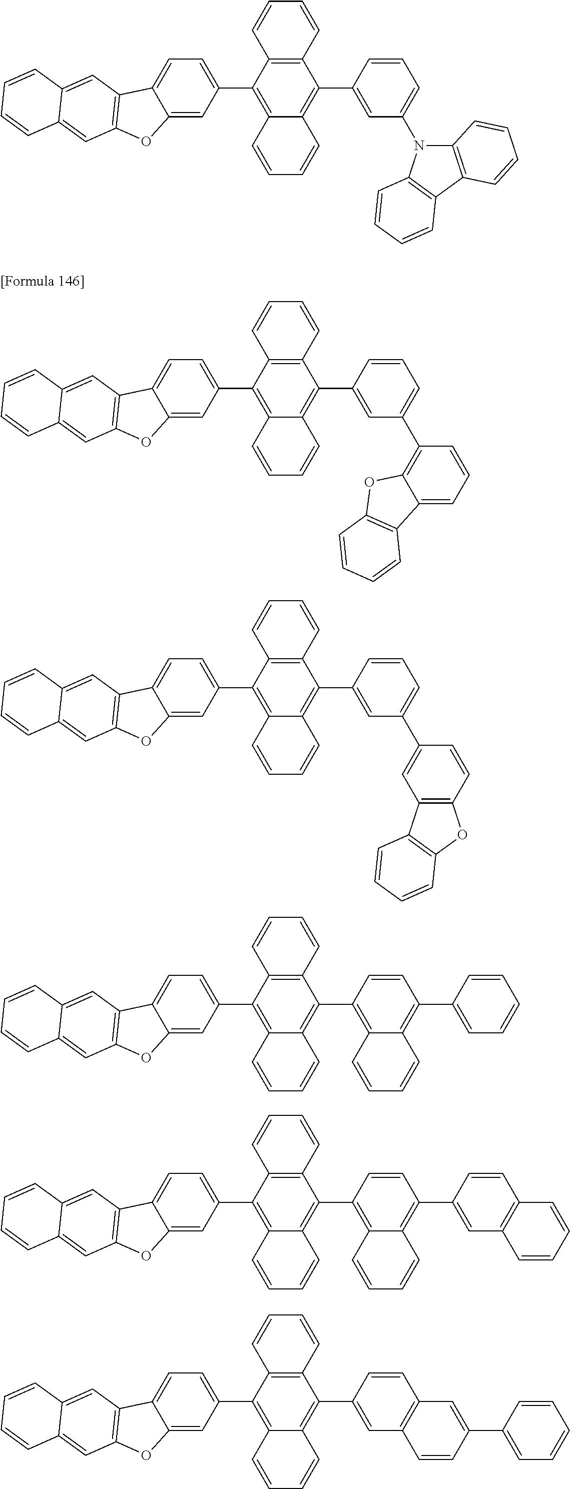

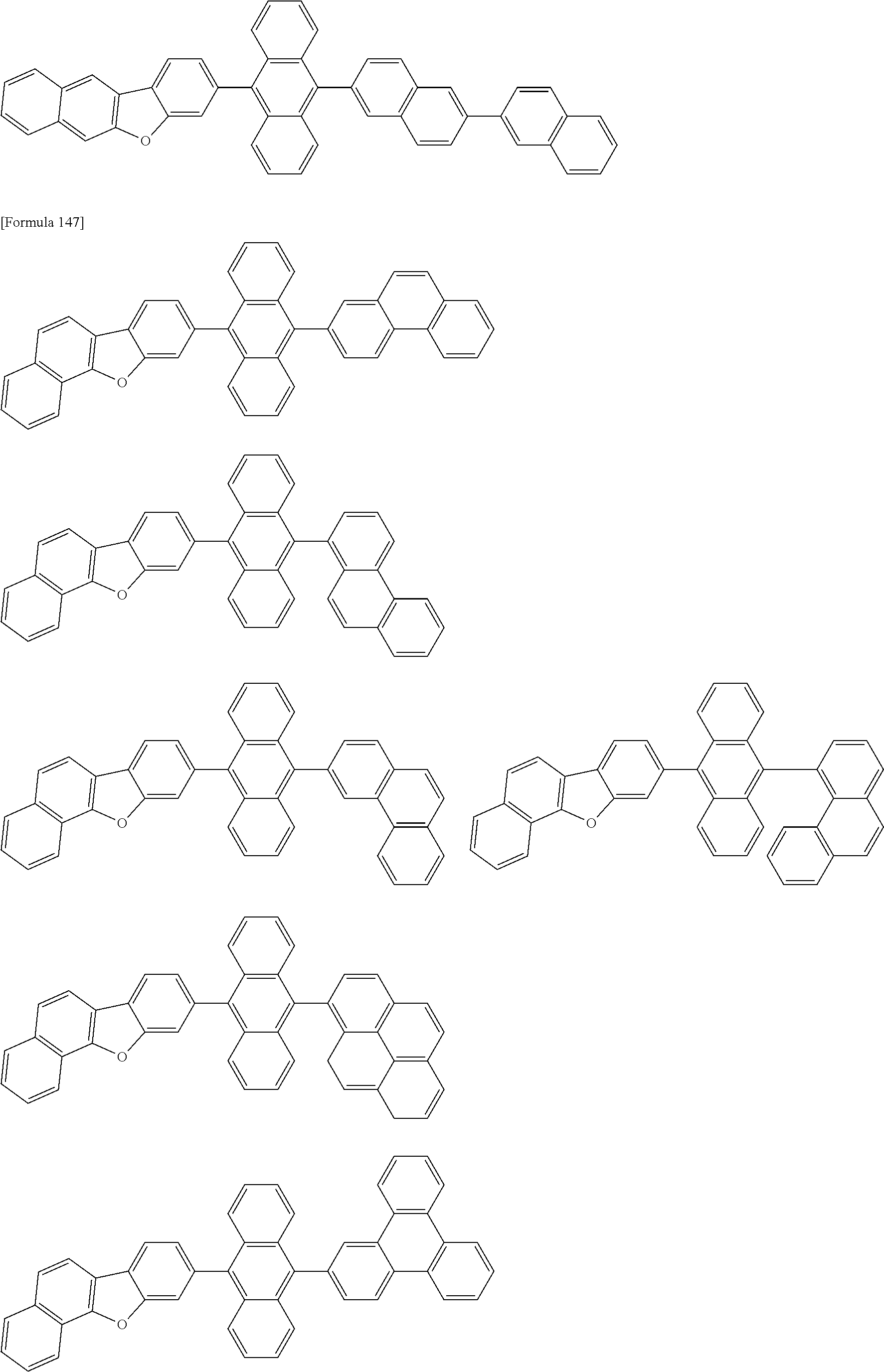

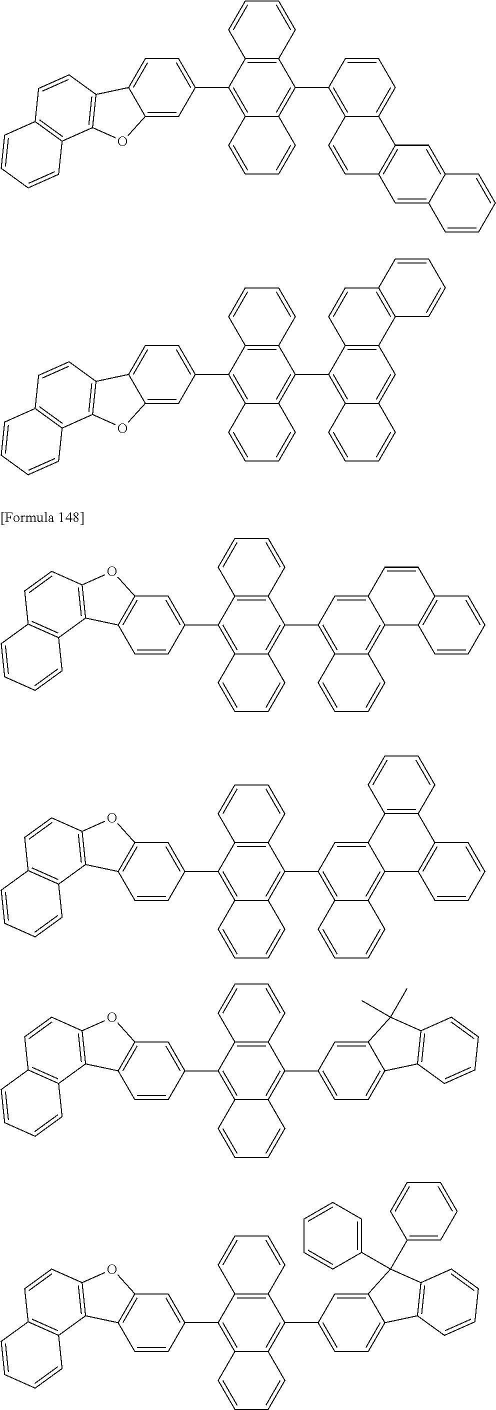

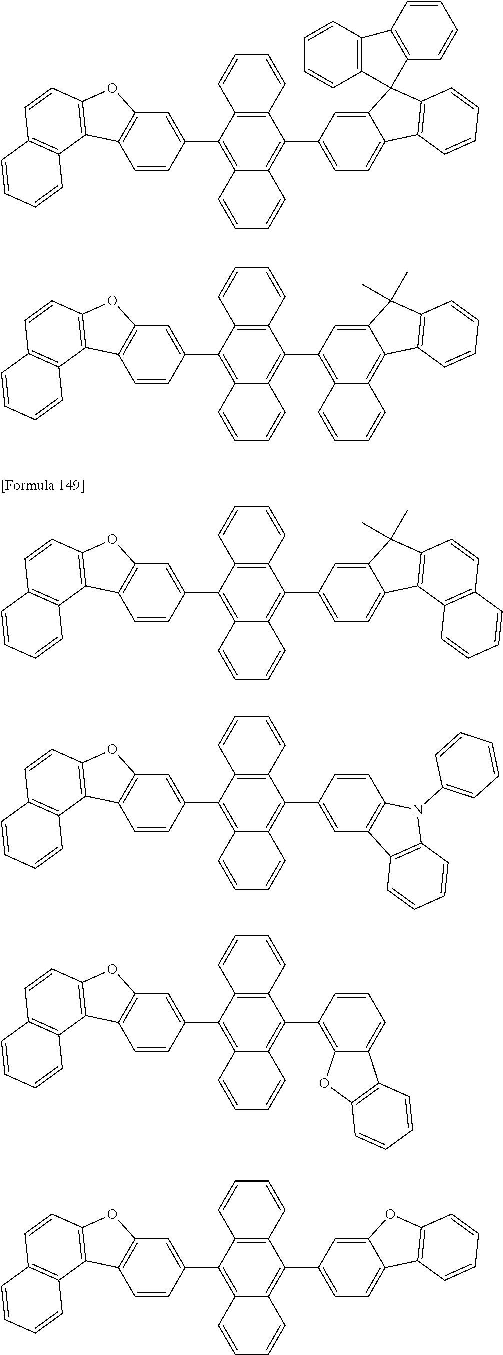

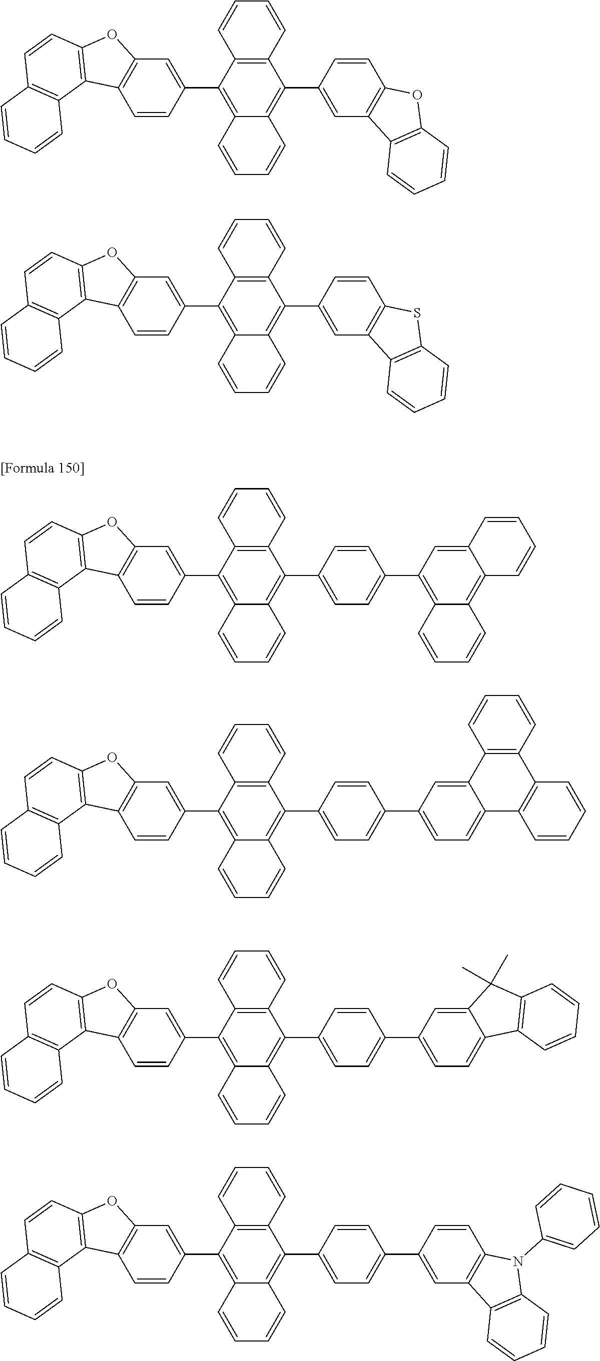

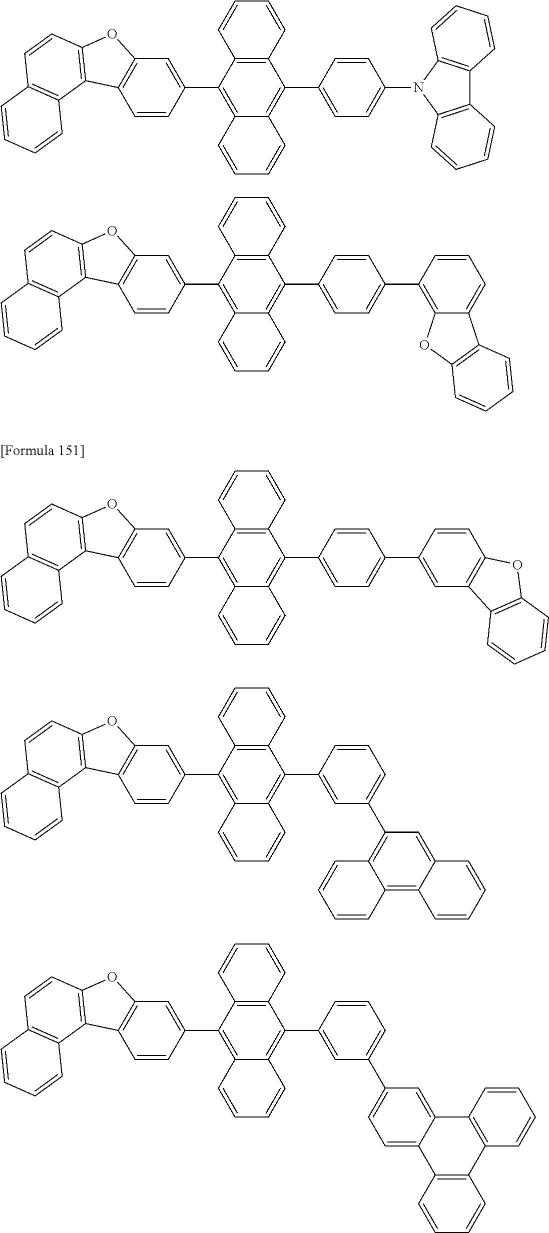

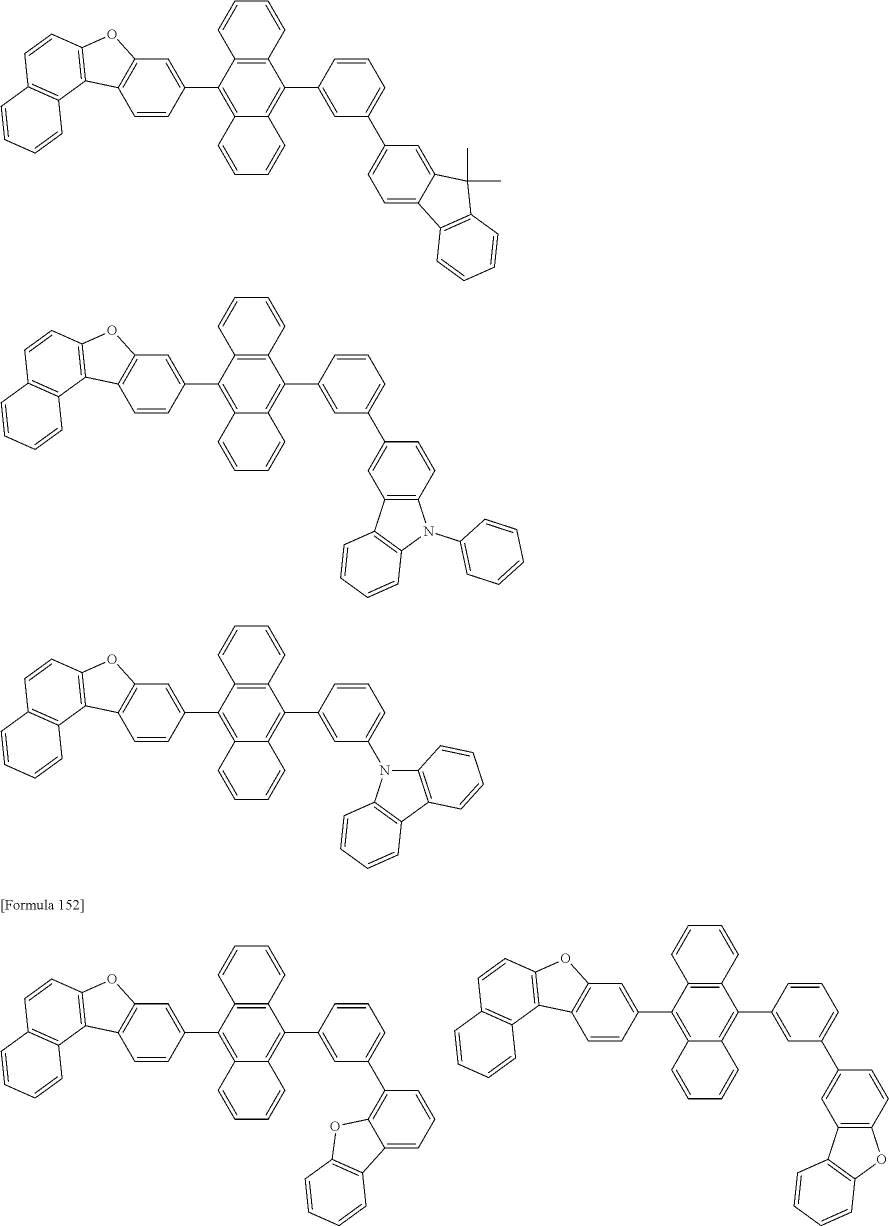 [SMILES: CC1(C)c2ccccc2-c2ccc(-c3cccc(-c4c5ccccc5c(-c5ccc6c(c5)oc5ccc7ccccc7c56)c5ccccc45)c3)cc21 ; c1ccc(-n2c3ccccc3c3cc(-c4cccc(-c5c6ccccc6c(-c6ccc7c(c6)oc6ccc8ccccc8c67)c6ccccc56)c4)ccc32)cc1 ; c1cc(-c2c3ccccc3c(-c3ccc4c(c3)oc3ccc5ccccc5c34)c3ccccc23)cc(-n2c3ccccc3c3ccccc32)c1 ; c1cc(-c2c3ccccc3c(-c3ccc4c(c3)oc3ccc5ccccc5c34)c3ccccc23)cc(-c2cccc3c2oc2ccccc23)c1 ; c1cc(-c2ccc3oc4ccccc4c3c2)cc(-c2c3ccccc3c(-c3ccc4c(c3)oc3ccc5ccccc5c34)c3ccccc23)c1]

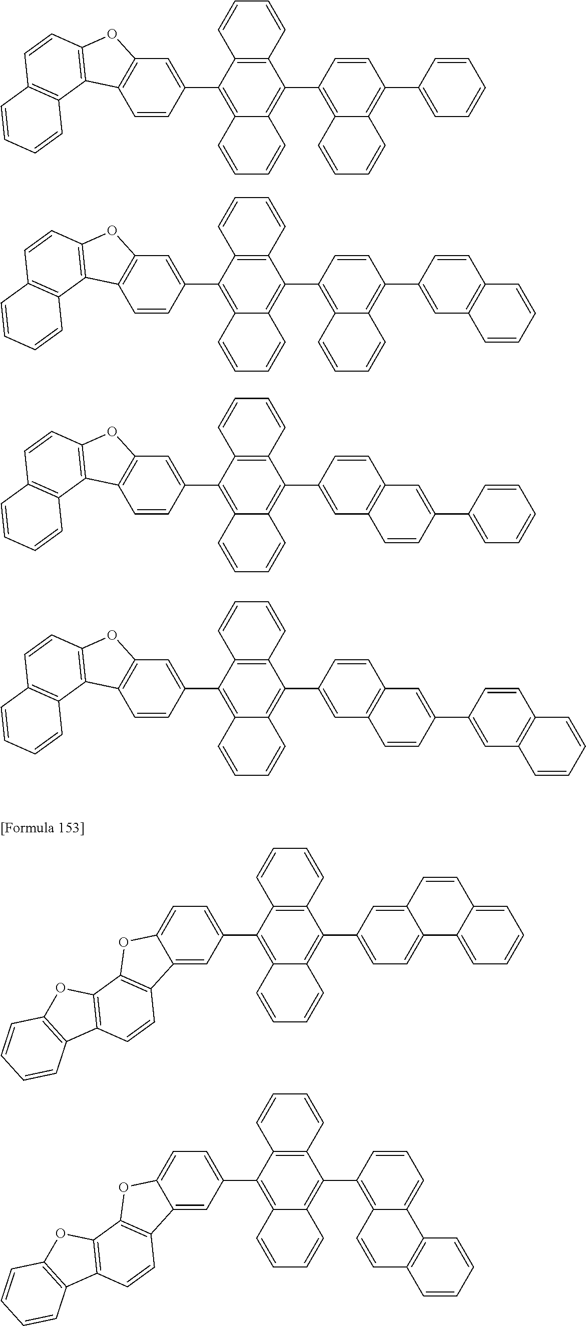

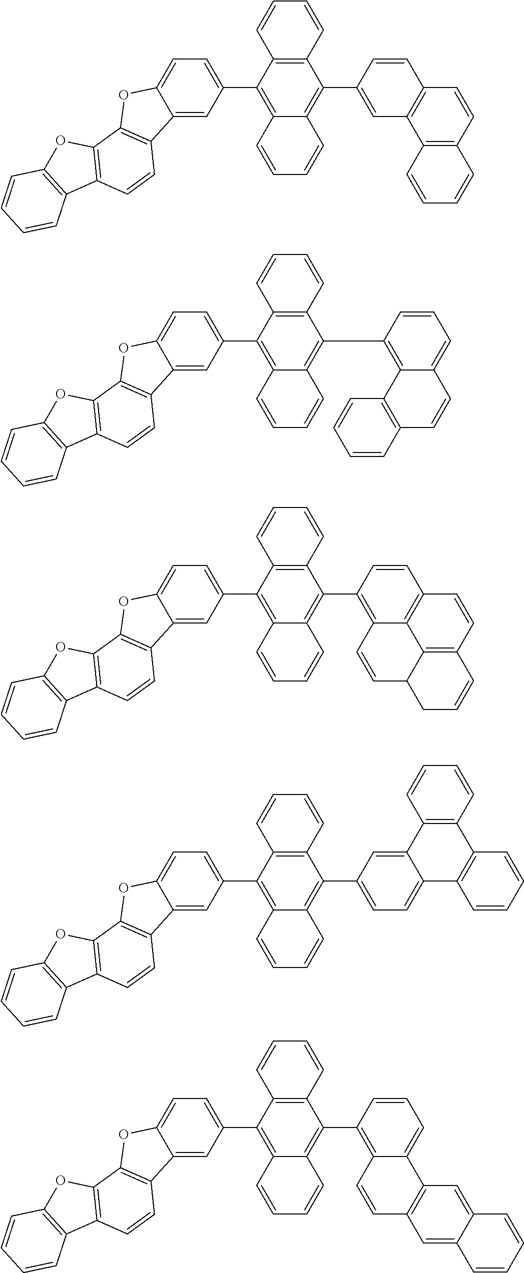 [SMILES: c1ccc2c(c1)ccc1ccc(-c3c4ccccc4c(-c4ccc5oc6c(ccc7c8ccccc8oc76)c5c4)c4ccccc34)cc12 ; c1ccc2c(c1)ccc1cccc(-c3c4ccccc4c(-c4ccc5oc6c(ccc7c8ccccc8oc76)c5c4)c4ccccc34)c12 ; C1=Cc2ccc3ccc(-c4c5ccccc5c(-c5ccc6oc7c(ccc8c9ccccc9oc87)c6c5)c5ccccc45)c4c3c2C(C=C4)C1 ; c1ccc2c(c1)oc1c2ccc2c3cc(-c4c5ccccc5c(-c5ccc6c7ccccc7c7ccccc7c6c5)c5ccccc45)ccc3oc21 ; c1ccc2cc3c(ccc4c(-c5c6ccccc6c(-c6ccc7oc8c(ccc9c%10ccccc%10oc98)c7c6)c6ccccc56)cccc43)cc2c1]

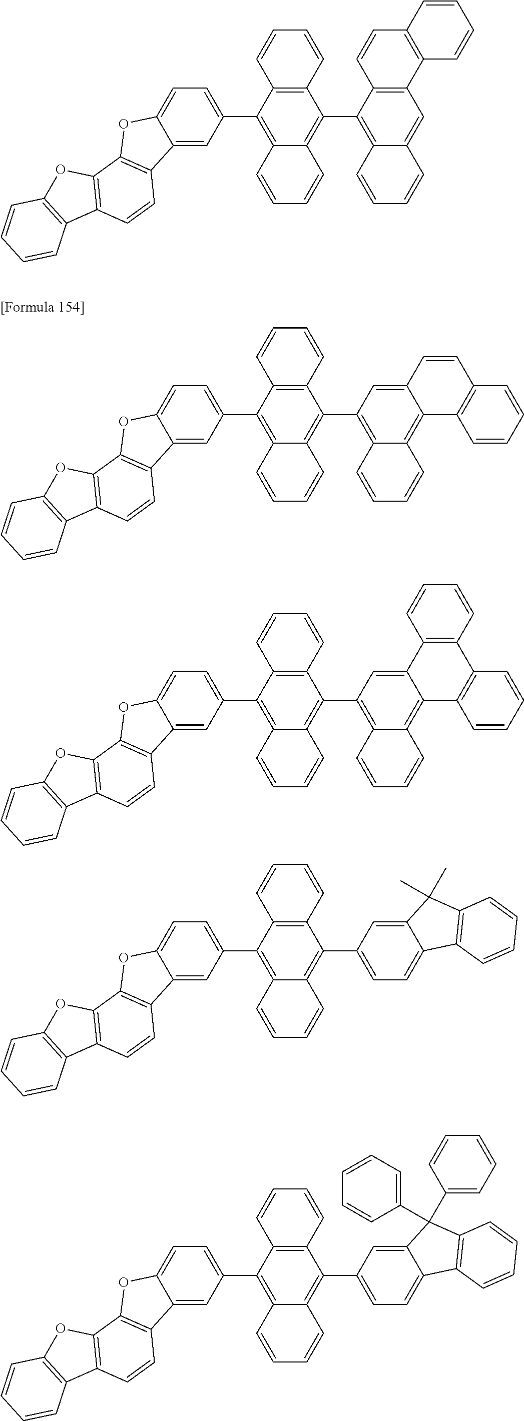 [SMILES: c1ccc2c(-c3c4ccccc4c(-c4ccc5oc6c(ccc7c8ccccc8oc76)c5c4)c4ccccc34)c3ccc4ccccc4c3cc2c1 ; c1ccc2c(c1)ccc1cc(-c3c4ccccc4c(-c4ccc5oc6c(ccc7c8ccccc8oc76)c5c4)c4ccccc34)c3ccccc3c12 ; c1ccc2c(c1)oc1c2ccc2c3cc(-c4c5ccccc5c(-c5cc6c7ccccc7c7ccccc7c6c6ccccc56)c5ccccc45)ccc3oc21 ; CC1(C)c2ccccc2-c2ccc(-c3c4ccccc4c(-c4ccc5oc6c(ccc7c8ccccc8oc76)c5c4)c4ccccc34)cc21 ; c1ccc(C2(c3ccccc3)c3ccccc3-c3ccc(-c4c5ccccc5c(-c5ccc6oc7c(ccc8c9ccccc9oc87)c6c5)c5ccccc45)cc32)cc1]

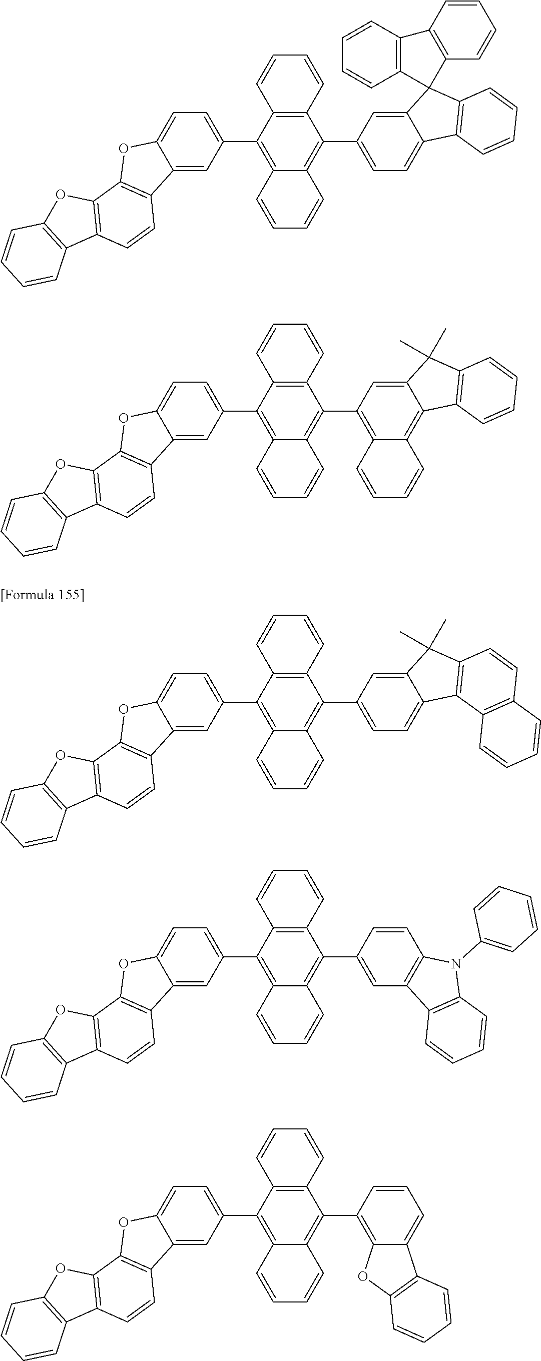 [SMILES: c1ccc2c(c1)-c1ccccc1C21c2ccccc2-c2ccc(-c3c4ccccc4c(-c4ccc5oc6c(ccc7c8ccccc8oc76)c5c4)c4ccccc34)cc21 ; CC1(C)c2ccccc2-c2c1cc(-c1c3ccccc3c(-c3ccc4oc5c(ccc6c7ccccc7oc65)c4c3)c3ccccc13)c1ccccc21 ; CC1(C)c2cc(-c3c4ccccc4c(-c4ccc5oc6c(ccc7c8ccccc8oc76)c5c4)c4ccccc34)ccc2-c2c1ccc1ccccc21 ; c1ccc(-n2c3ccccc3c3cc(-c4c5ccccc5c(-c5ccc6oc7c(ccc8c9ccccc9oc87)c6c5)c5ccccc45)ccc32)cc1 ; c1ccc2c(c1)oc1c(-c3c4ccccc4c(-c4ccc5oc6c(ccc7c8ccccc8oc76)c5c4)c4ccccc34)cccc12]

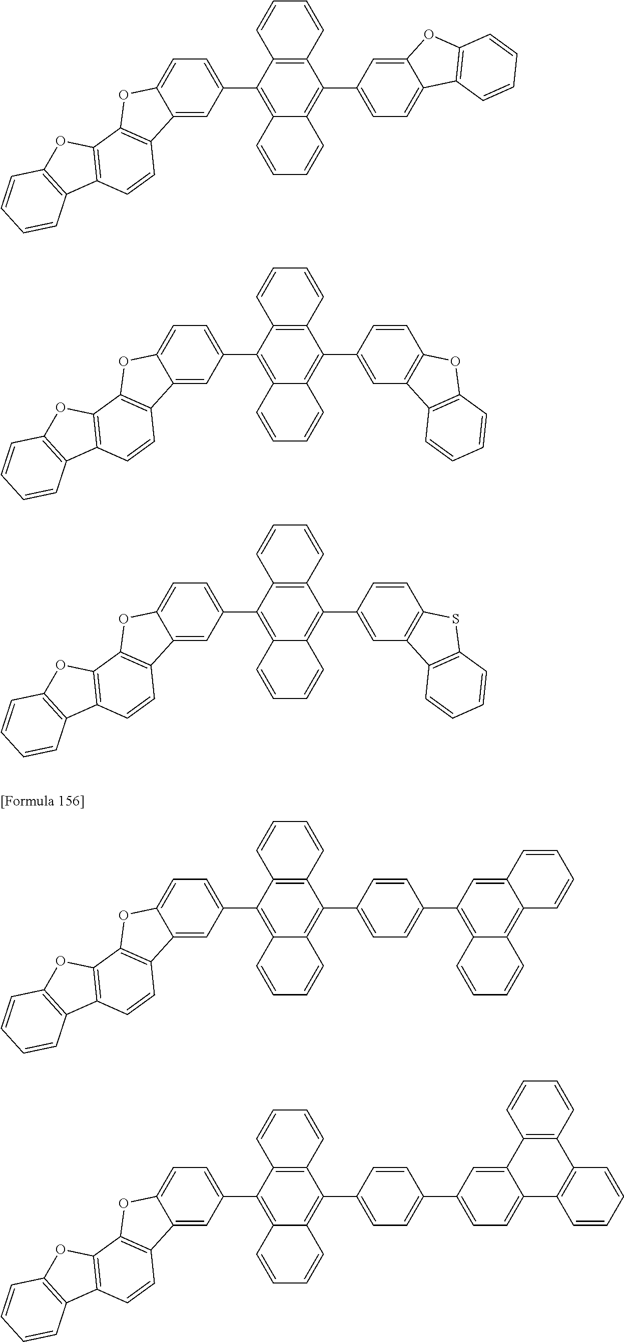 [SMILES: c1ccc2c(c1)oc1cc(-c3c4ccccc4c(-c4ccc5oc6c(ccc7c8ccccc8oc76)c5c4)c4ccccc34)ccc12 ; c1ccc2c(c1)oc1ccc(-c3c4ccccc4c(-c4ccc5oc6c(ccc7c8ccccc8oc76)c5c4)c4ccccc34)cc12 ; c1ccc2c(c1)oc1c2ccc2c3cc(-c4c5ccccc5c(-c5ccc6sc7ccccc7c6c5)c5ccccc45)ccc3oc21 ; c1ccc2c(c1)cc(-c1ccc(-c3c4ccccc4c(-c4ccc5oc6c(ccc7c8ccccc8oc76)c5c4)c4ccccc34)cc1)c1ccccc12 ; c1ccc2c(c1)oc1c2ccc2c3cc(-c4c5ccccc5c(-c5ccc(-c6ccc7c8ccccc8c8ccccc8c7c6)cc5)c5ccccc45)ccc3oc21]

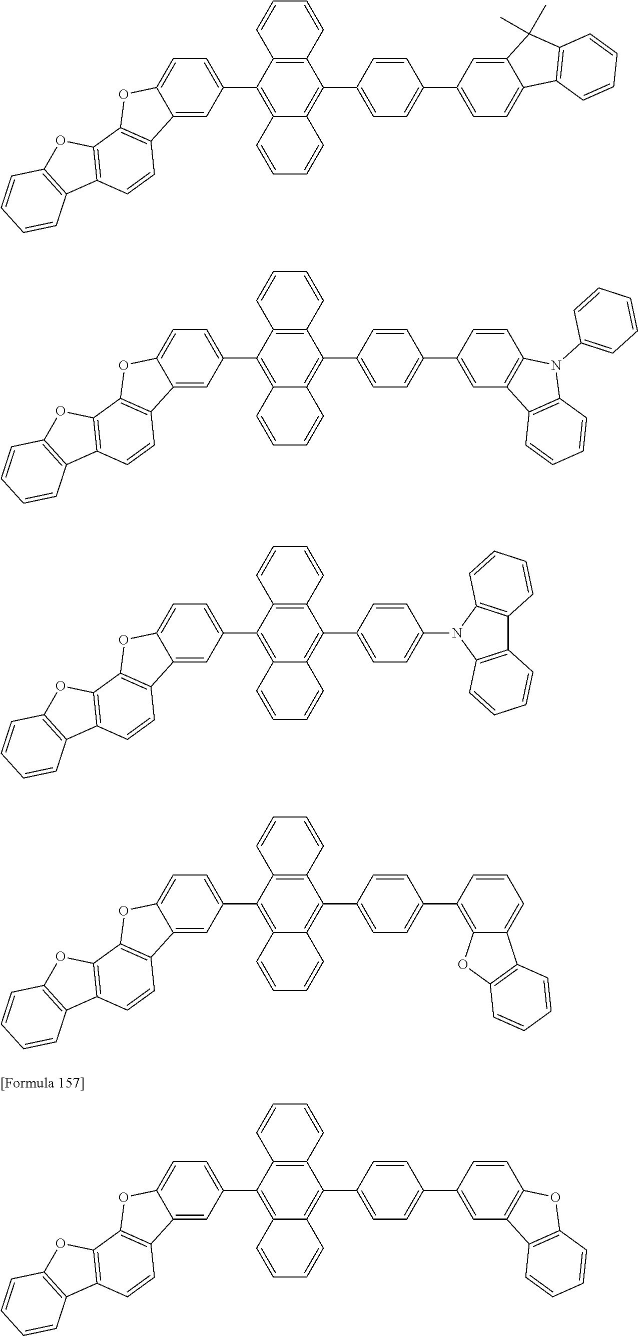 [SMILES: CC1(C)c2ccccc2-c2ccc(-c3ccc(-c4c5ccccc5c(-c5ccc6oc7c(ccc8c9ccccc9oc87)c6c5)c5ccccc45)cc3)cc21 ; c1ccc(-n2c3ccccc3c3cc(-c4ccc(-c5c6ccccc6c(-c6ccc7oc8c(ccc9c%10ccccc%10oc98)c7c6)c6ccccc56)cc4)ccc32)cc1 ; c1ccc2c(c1)oc1c2ccc2c3cc(-c4c5ccccc5c(-c5ccc(-n6c7ccccc7c7ccccc76)cc5)c5ccccc45)ccc3oc21 ; c1ccc2c(c1)oc1c(-c3ccc(-c4c5ccccc5c(-c5ccc6oc7c(ccc8c9ccccc9oc87)c6c5)c5ccccc45)cc3)cccc12 ; c1ccc2c(c1)oc1ccc(-c3ccc(-c4c5ccccc5c(-c5ccc6oc7c(ccc8c9ccccc9oc87)c6c5)c5ccccc45)cc3)cc12]

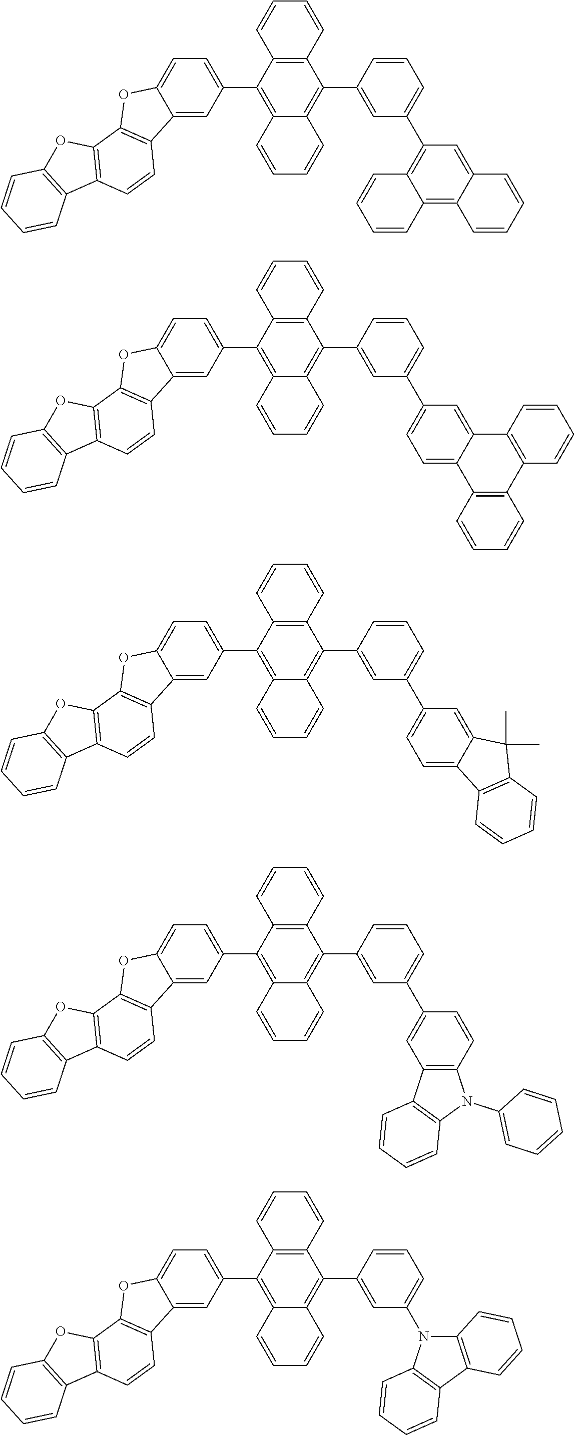 [SMILES: c1cc(-c2c3ccccc3c(-c3ccc4oc5c(ccc6c7ccccc7oc65)c4c3)c3ccccc23)cc(-c2cc3ccccc3c3ccccc23)c1 ; c1cc(-c2ccc3c4ccccc4c4ccccc4c3c2)cc(-c2c3ccccc3c(-c3ccc4oc5c(ccc6c7ccccc7oc65)c4c3)c3ccccc23)c1 ; CC1(C)c2ccccc2-c2ccc(-c3cccc(-c4c5ccccc5c(-c5ccc6oc7c(ccc8c9ccccc9oc87)c6c5)c5ccccc45)c3)cc21 ; c1ccc(-n2c3ccccc3c3cc(-c4cccc(-c5c6ccccc6c(-c6ccc7oc8c(ccc9c%10ccccc%10oc98)c7c6)c6ccccc56)c4)ccc32)cc1 ; c1cc(-c2c3ccccc3c(-c3ccc4oc5c(ccc6c7ccccc7oc65)c4c3)c3ccccc23)cc(-n2c3ccccc3c3ccccc32)c1]

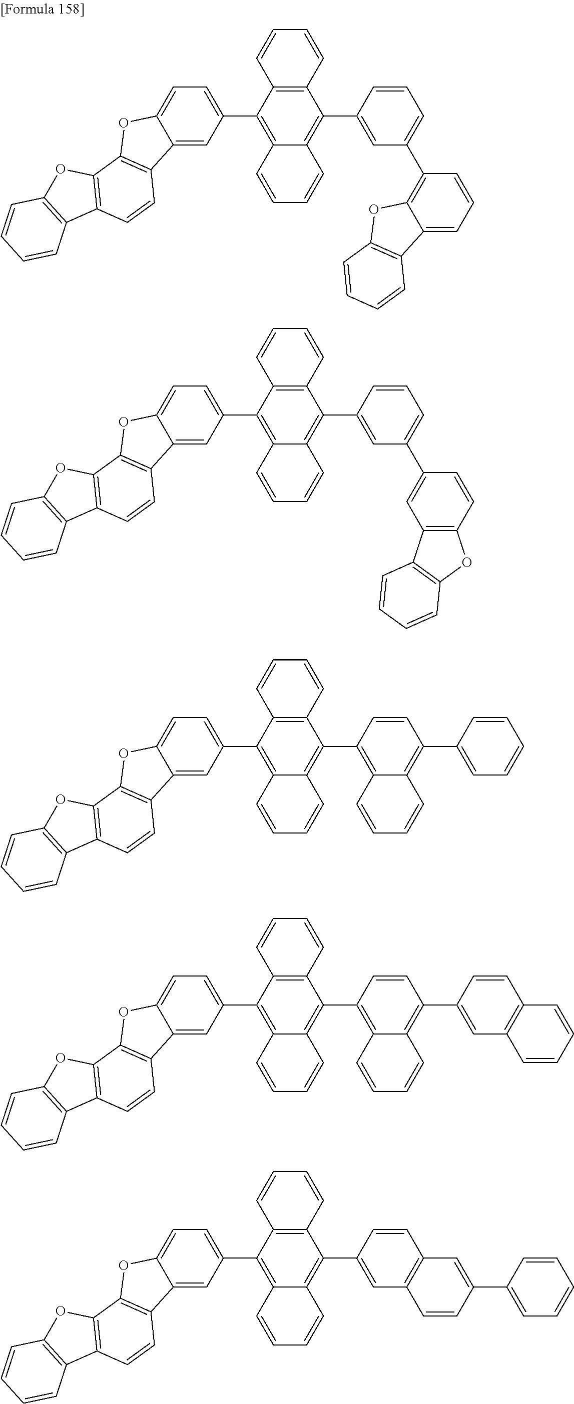 [SMILES: c1cc(-c2c3ccccc3c(-c3ccc4oc5c(ccc6c7ccccc7oc65)c4c3)c3ccccc23)cc(-c2cccc3c2oc2ccccc23)c1 ; c1cc(-c2ccc3oc4ccccc4c3c2)cc(-c2c3ccccc3c(-c3ccc4oc5c(ccc6c7ccccc7oc65)c4c3)c3ccccc23)c1 ; c1ccc(-c2ccc(-c3c4ccccc4c(-c4ccc5oc6c(ccc7c8ccccc8oc76)c5c4)c4ccccc34)c3ccccc23)cc1 ; c1ccc2cc(-c3ccc(-c4c5ccccc5c(-c5ccc6oc7c(ccc8c9ccccc9oc87)c6c5)c5ccccc45)c4ccccc34)ccc2c1 ; c1ccc(-c2ccc3cc(-c4c5ccccc5c(-c5ccc6oc7c(ccc8c9ccccc9oc87)c6c5)c5ccccc45)ccc3c2)cc1]

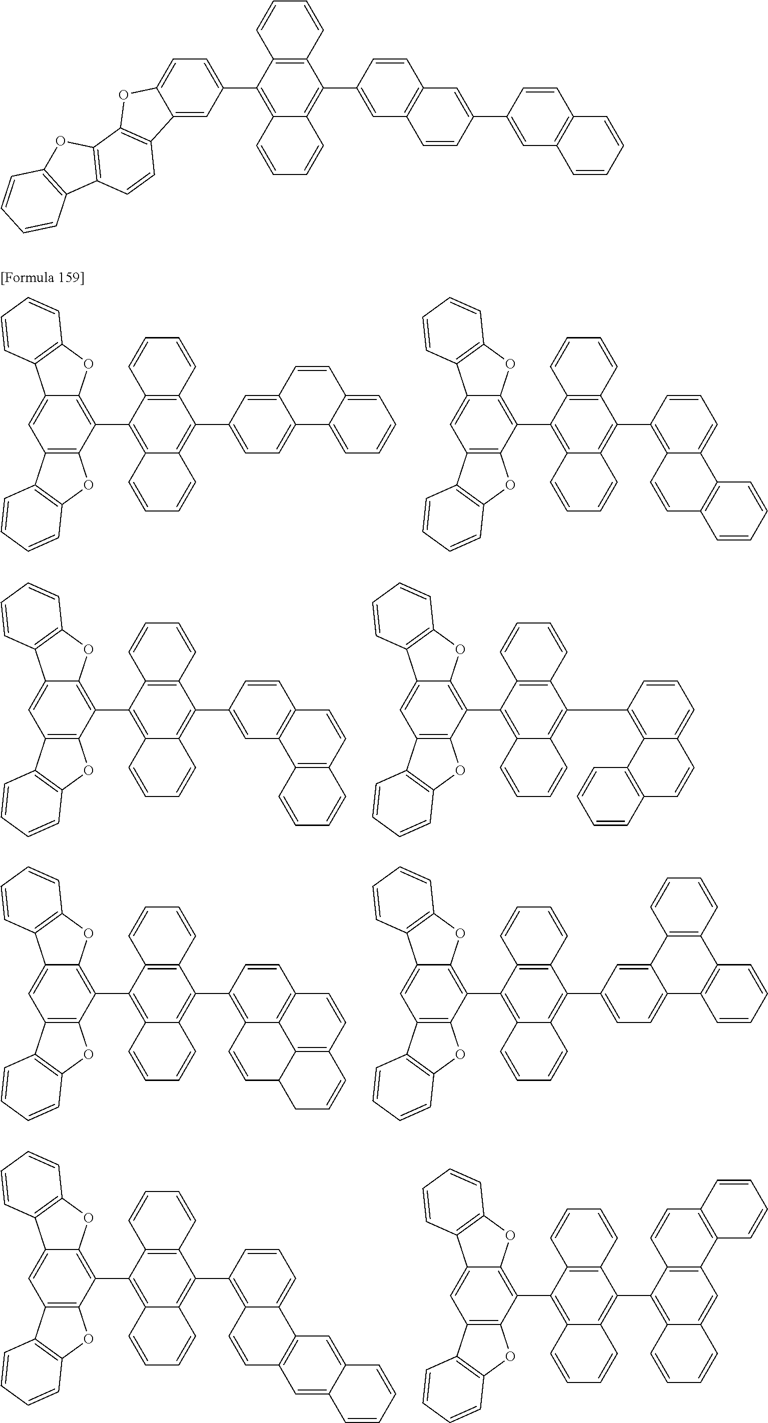

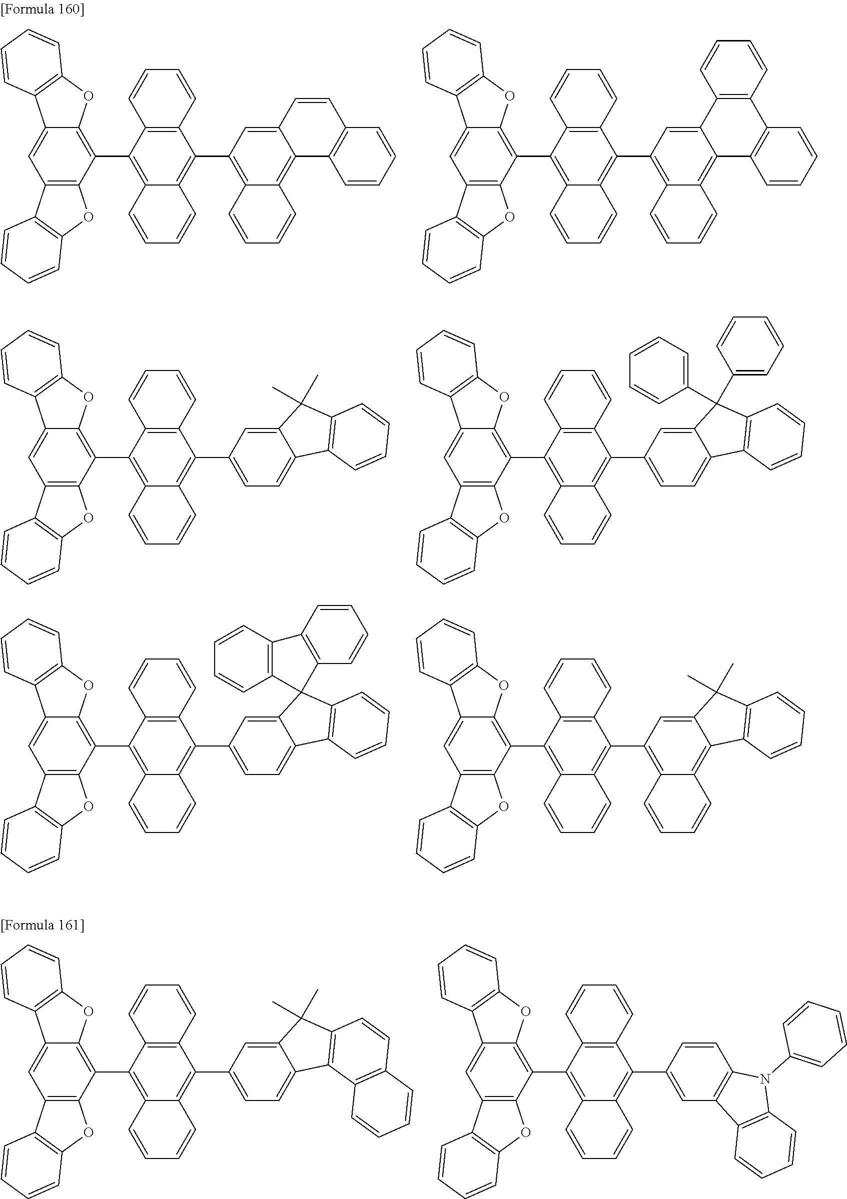

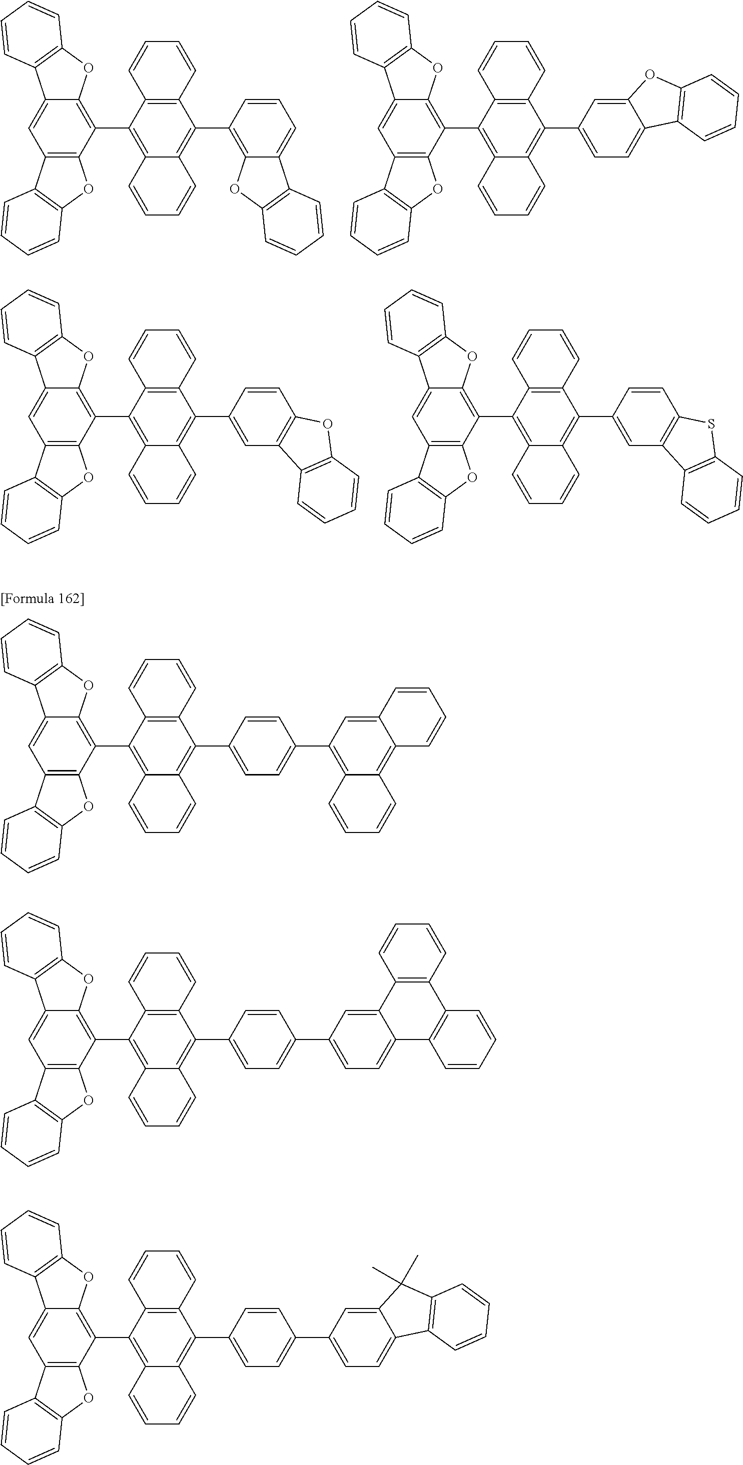

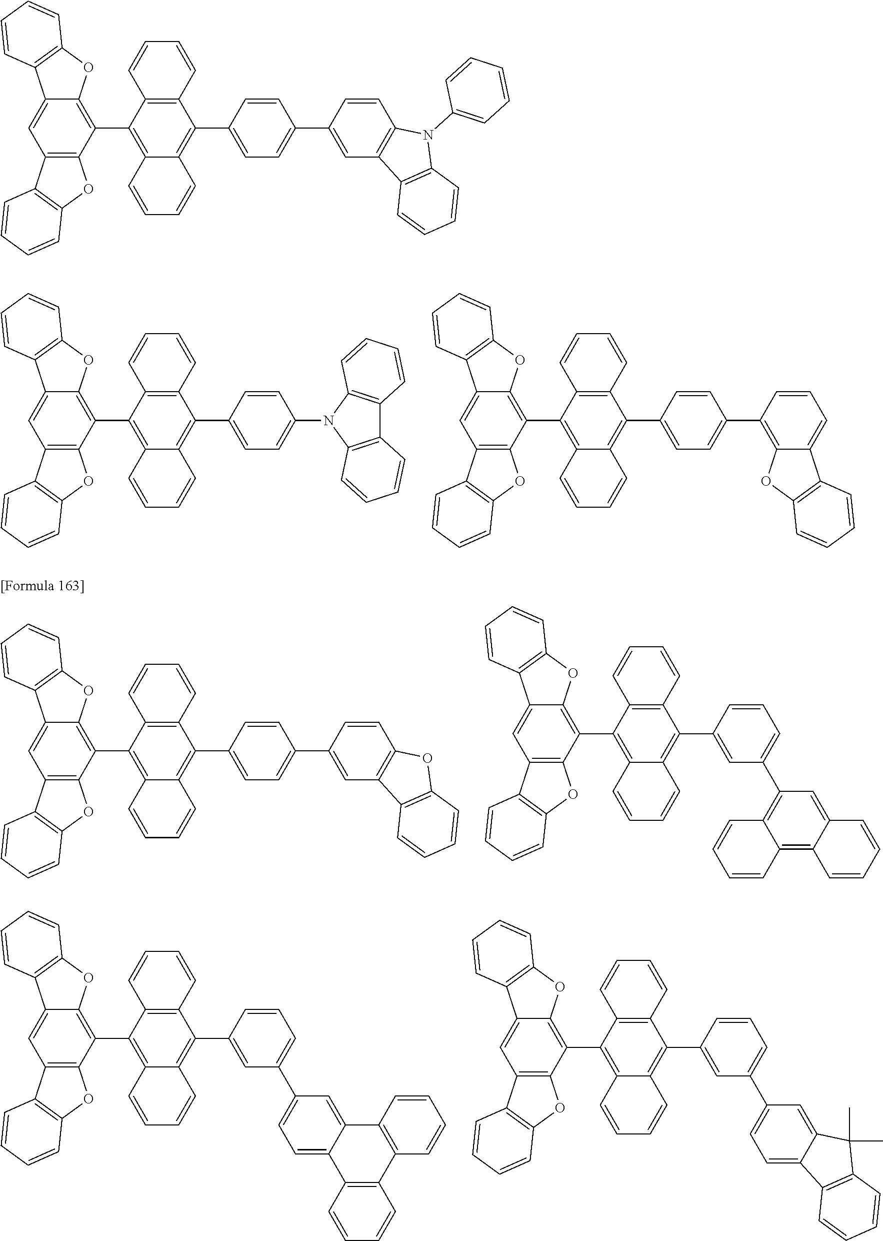

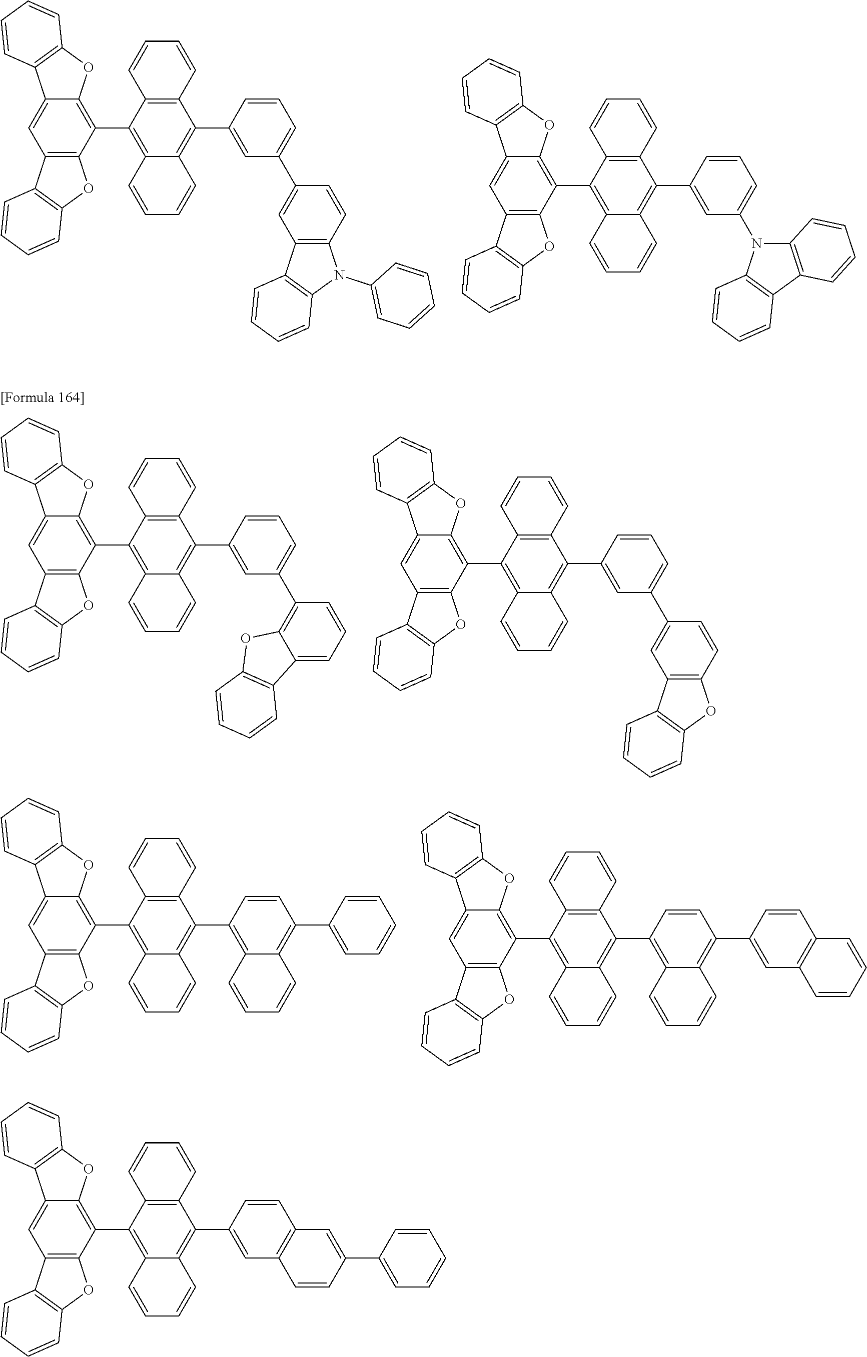

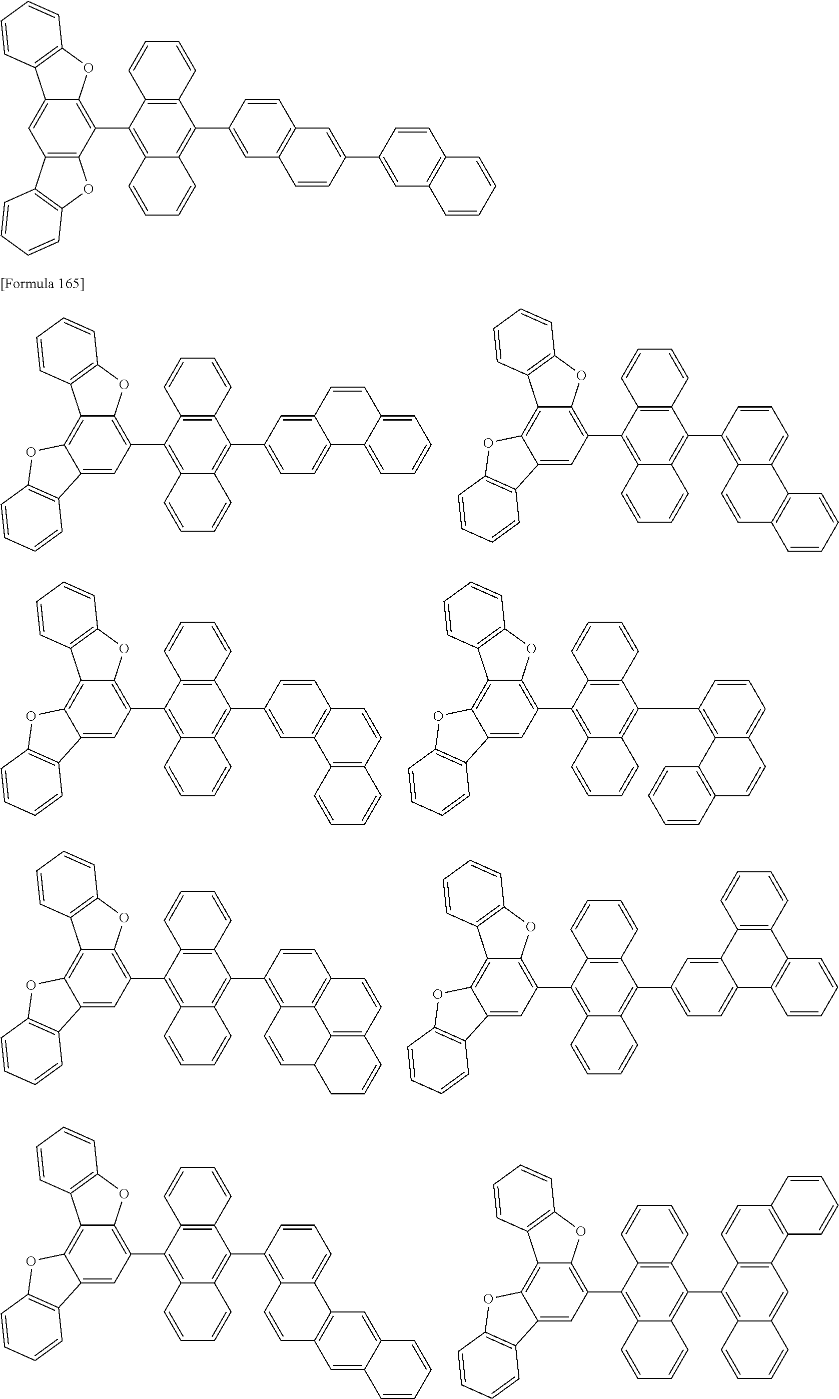

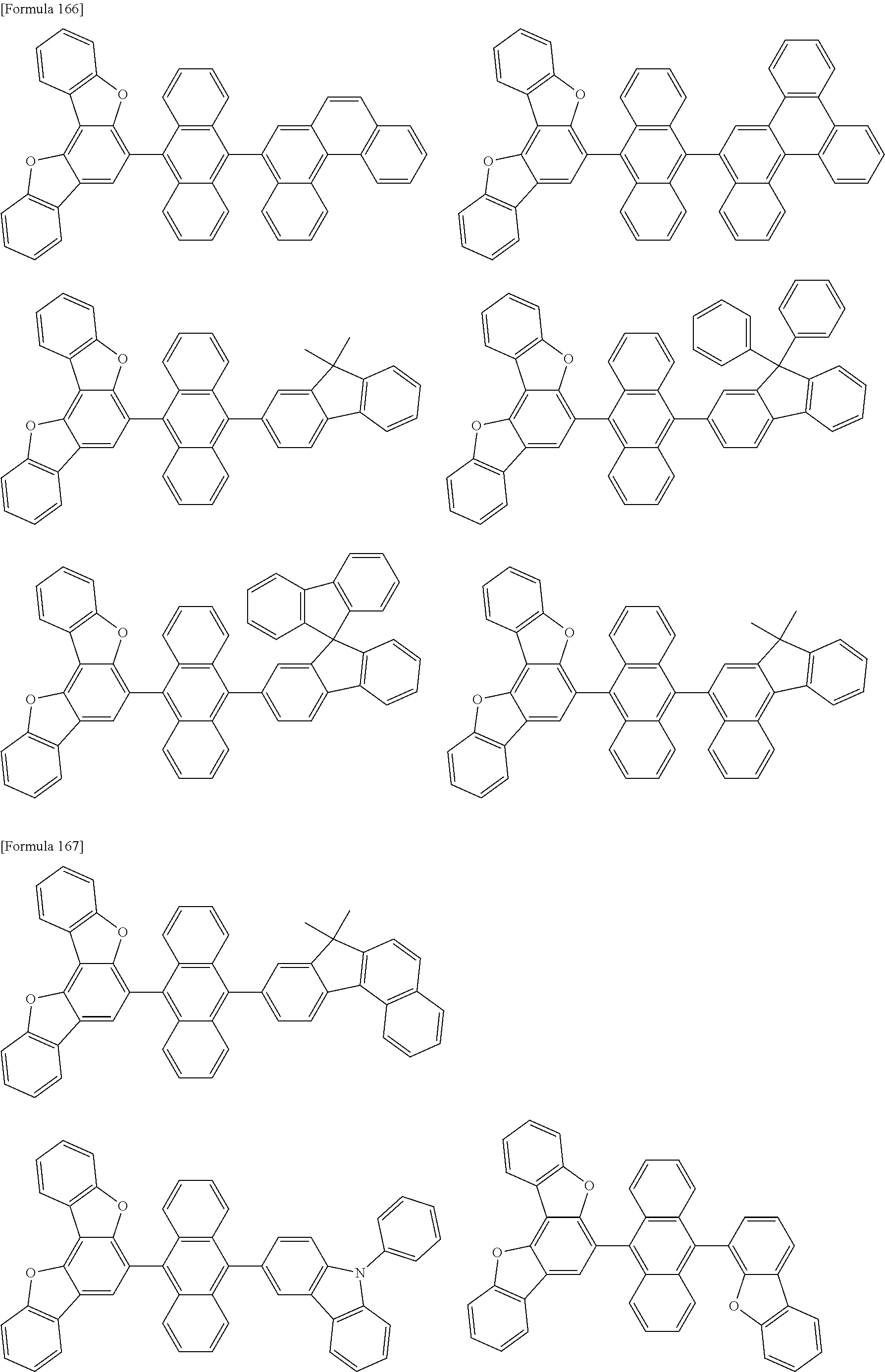

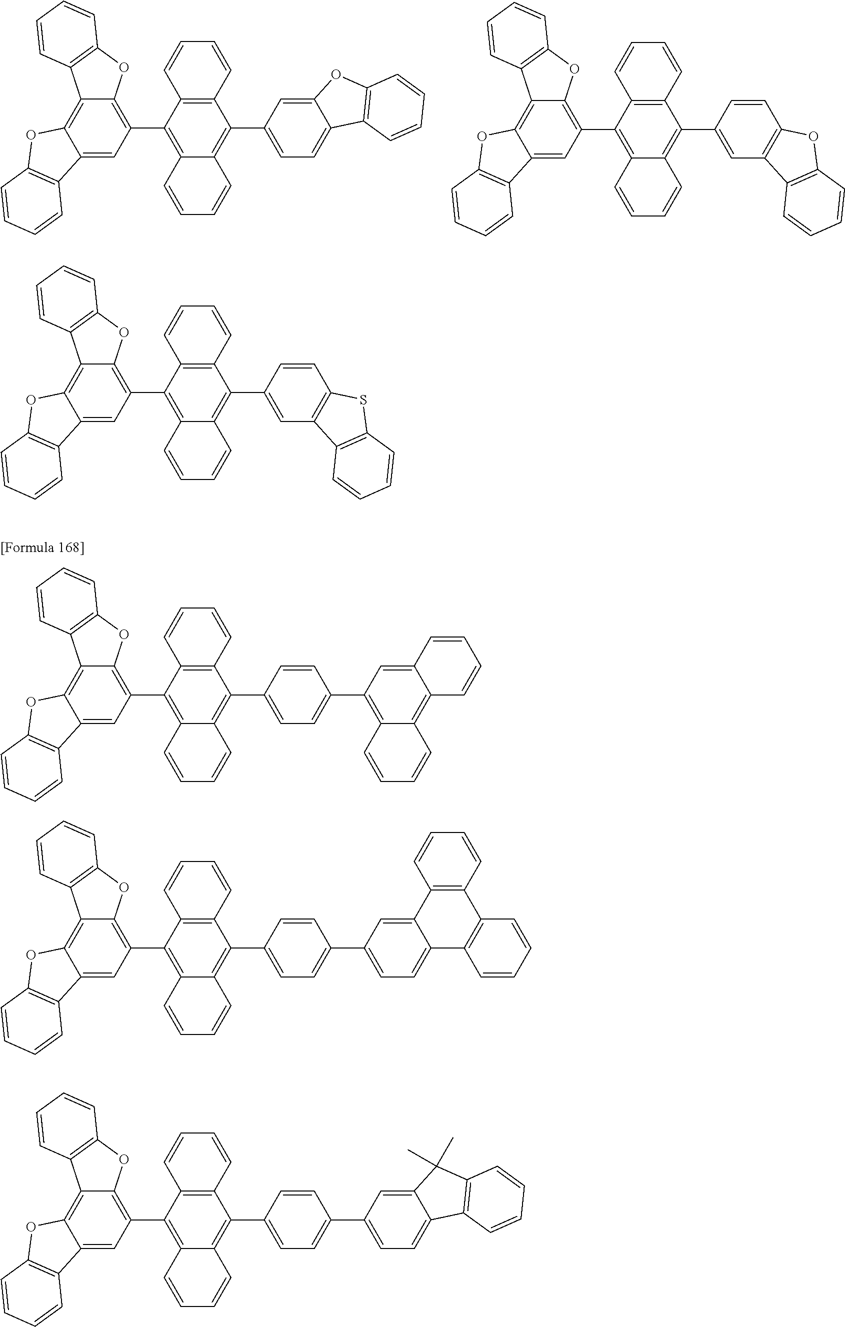

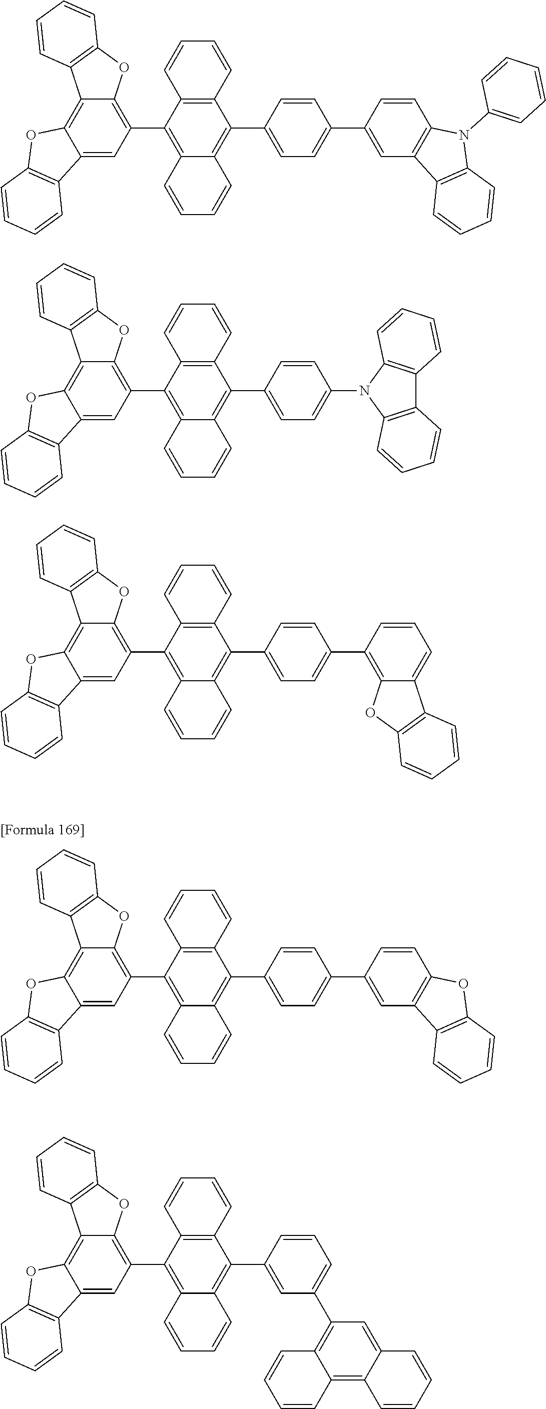

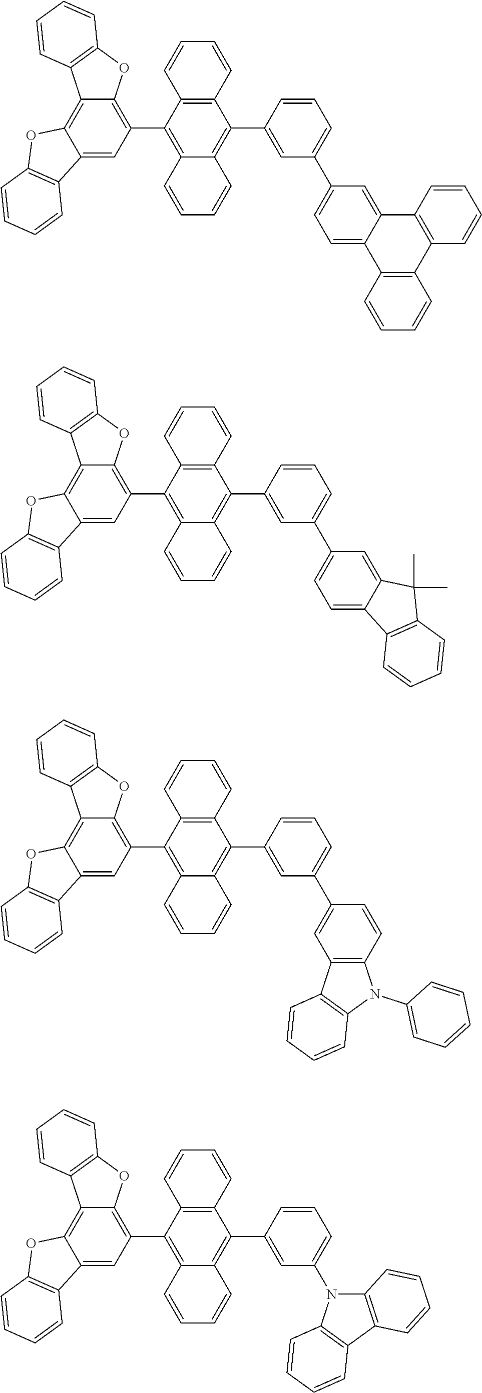 [SMILES: c1cc(-c2ccc3c4ccccc4c4ccccc4c3c2)cc(-c2c3ccccc3c(-c3cc4c5ccccc5oc4c4c3oc3ccccc34)c3ccccc23)c1 ; CC1(C)c2ccccc2-c2ccc(-c3cccc(-c4c5ccccc5c(-c5cc6c7ccccc7oc6c6c5oc5ccccc56)c5ccccc45)c3)cc21 ; c1ccc(-n2c3ccccc3c3cc(-c4cccc(-c5c6ccccc6c(-c6cc7c8ccccc8oc7c7c6oc6ccccc67)c6ccccc56)c4)ccc32)cc1 ; c1cc(-c2c3ccccc3c(-c3cc4c5ccccc5oc4c4c3oc3ccccc34)c3ccccc23)cc(-n2c3ccccc3c3ccccc32)c1]

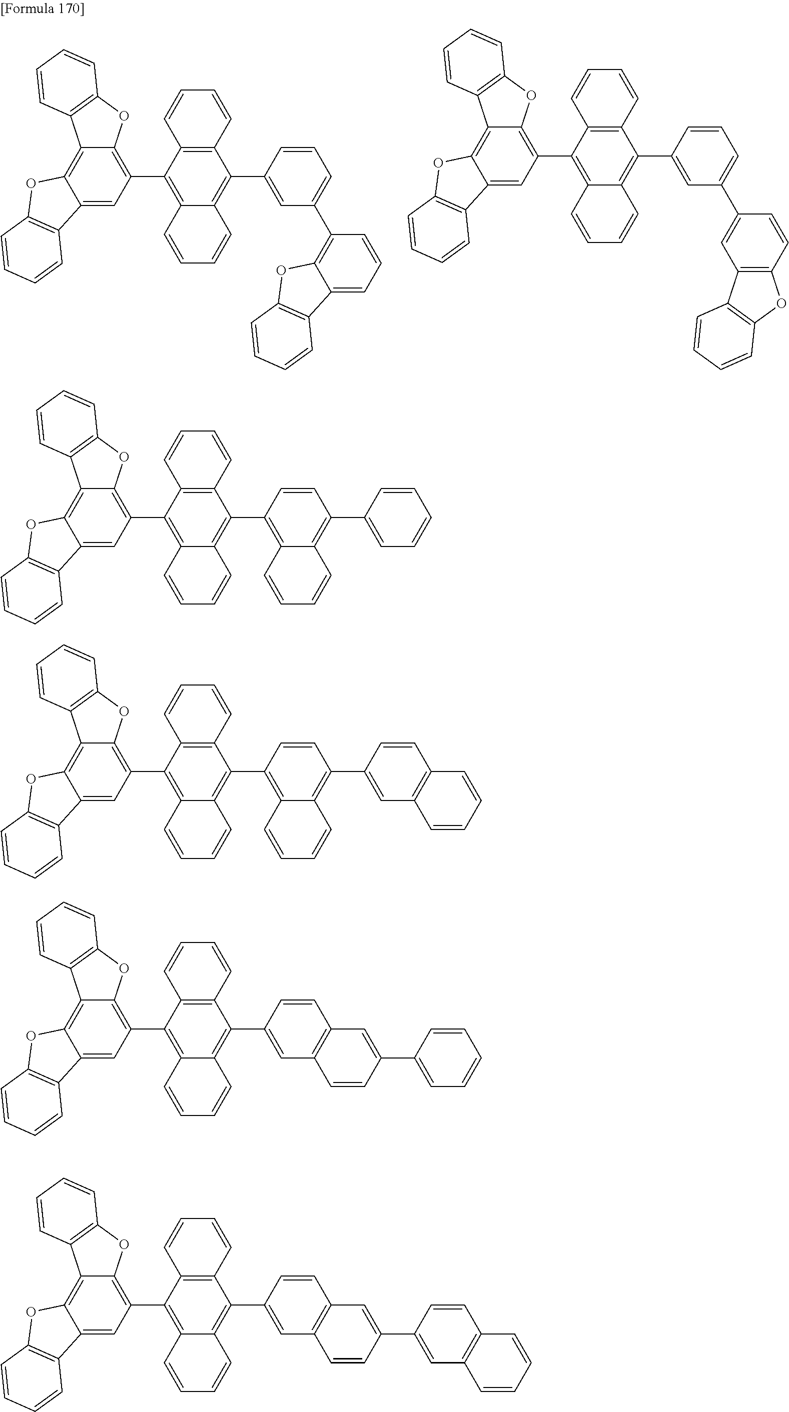

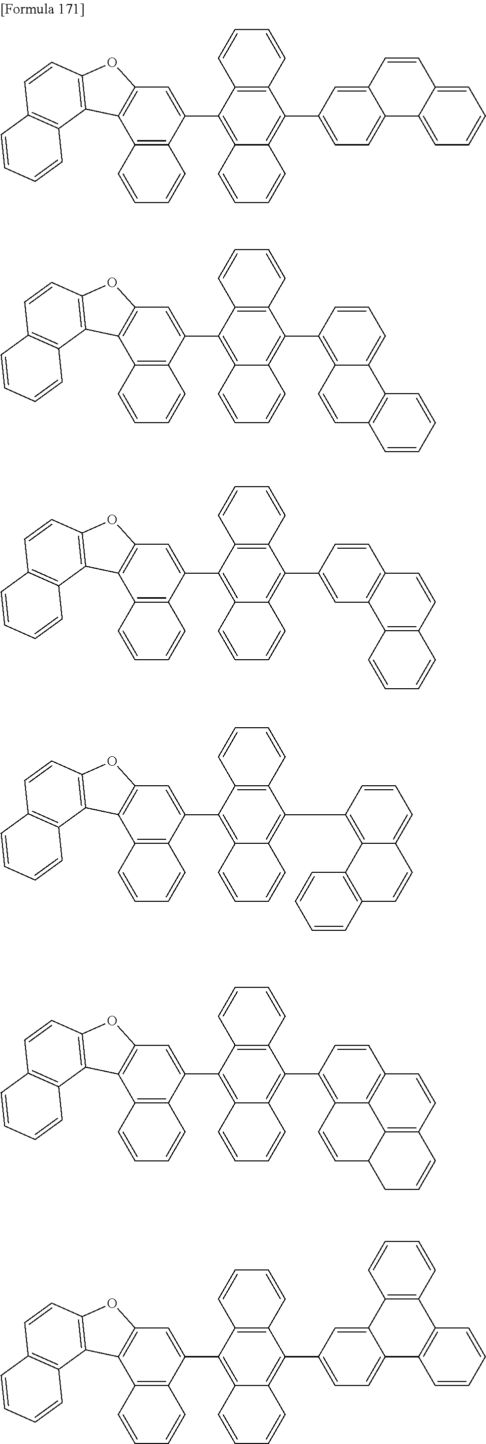

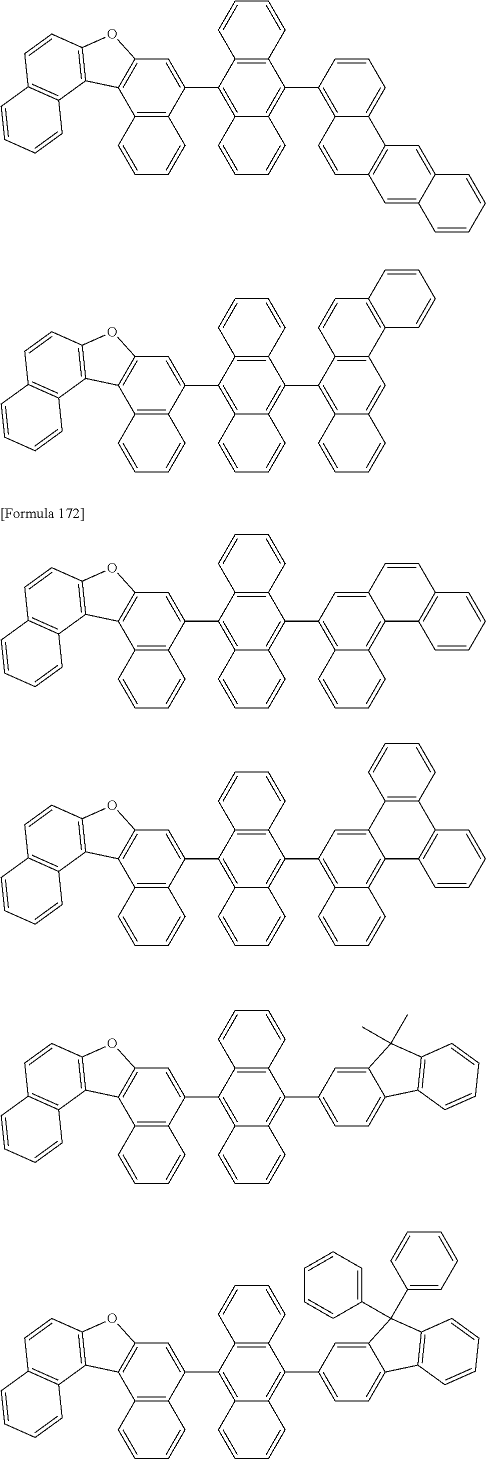

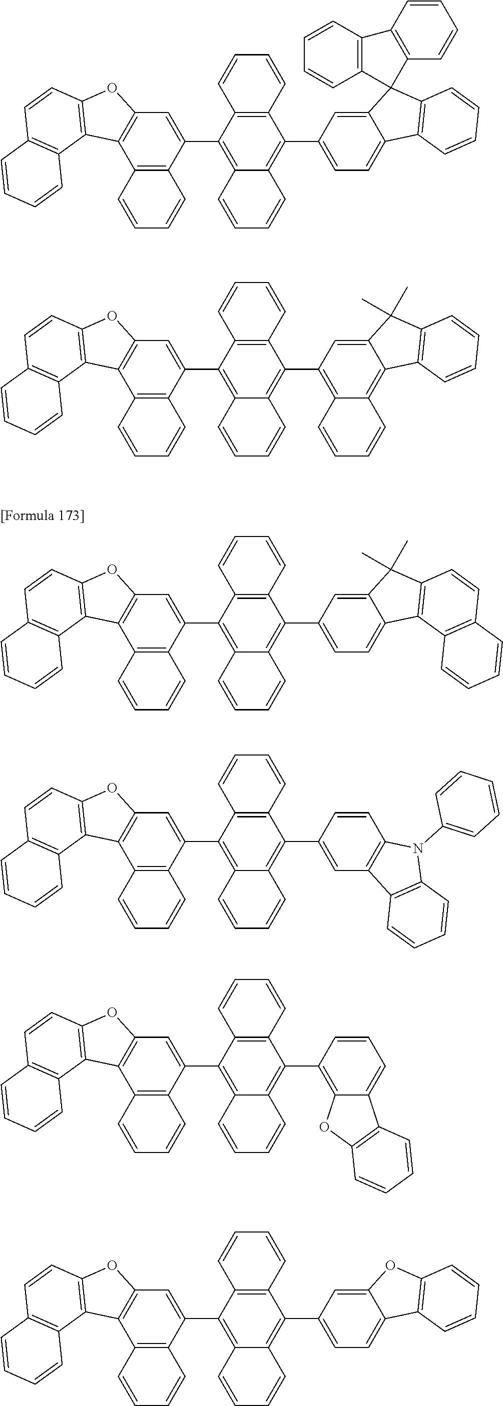

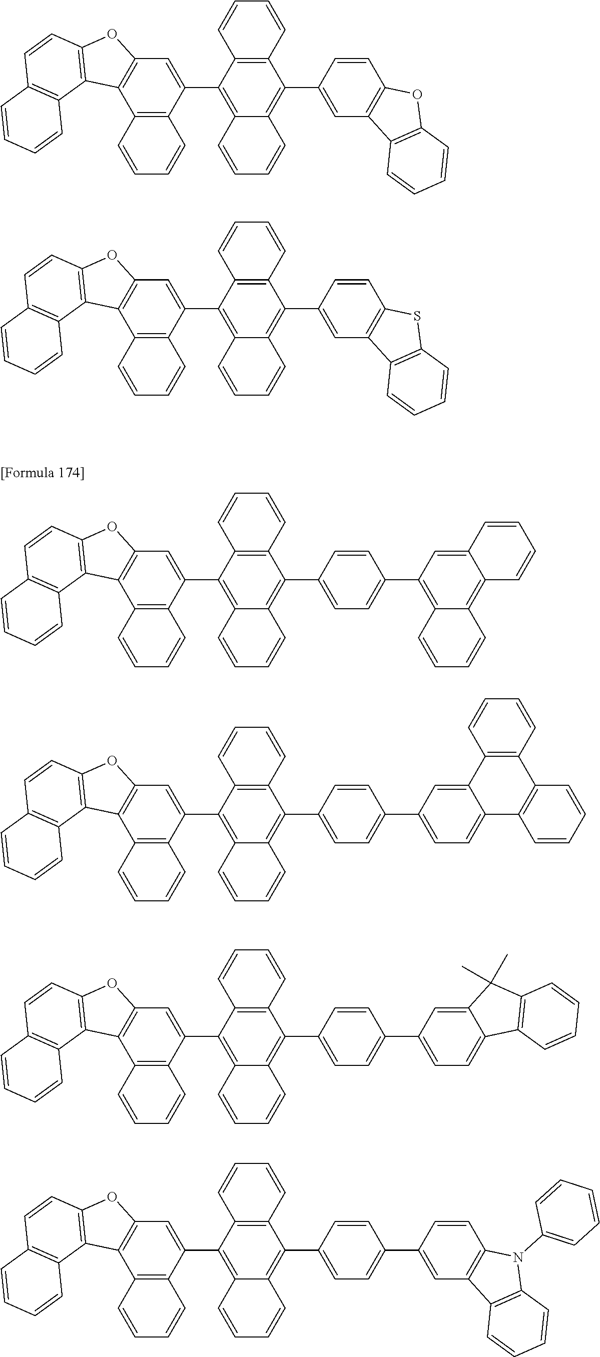

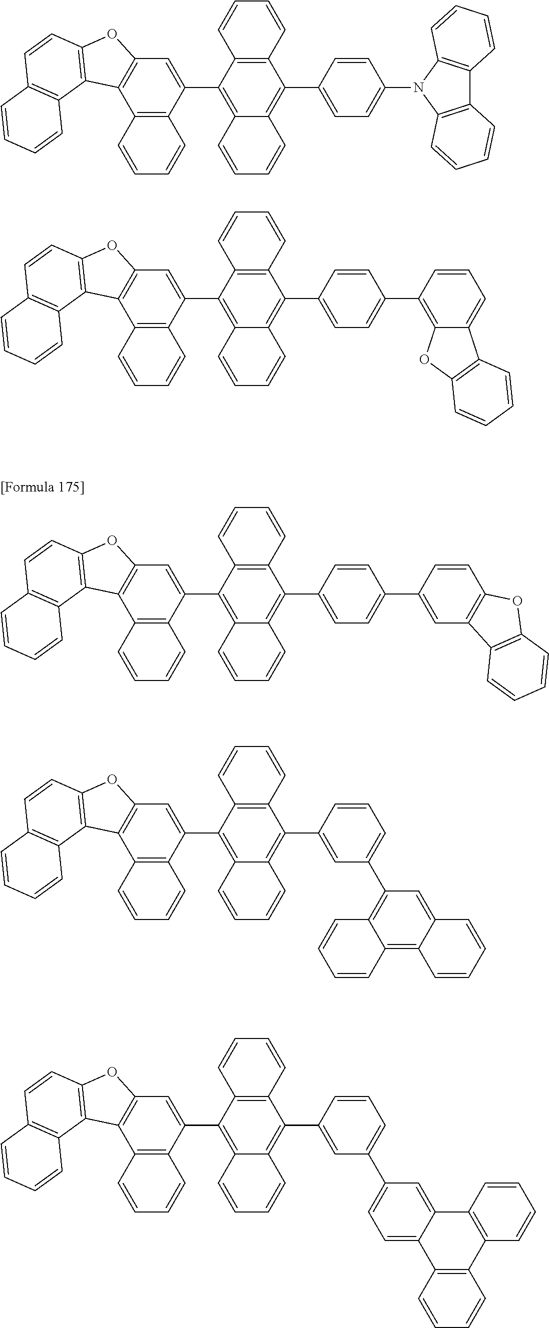

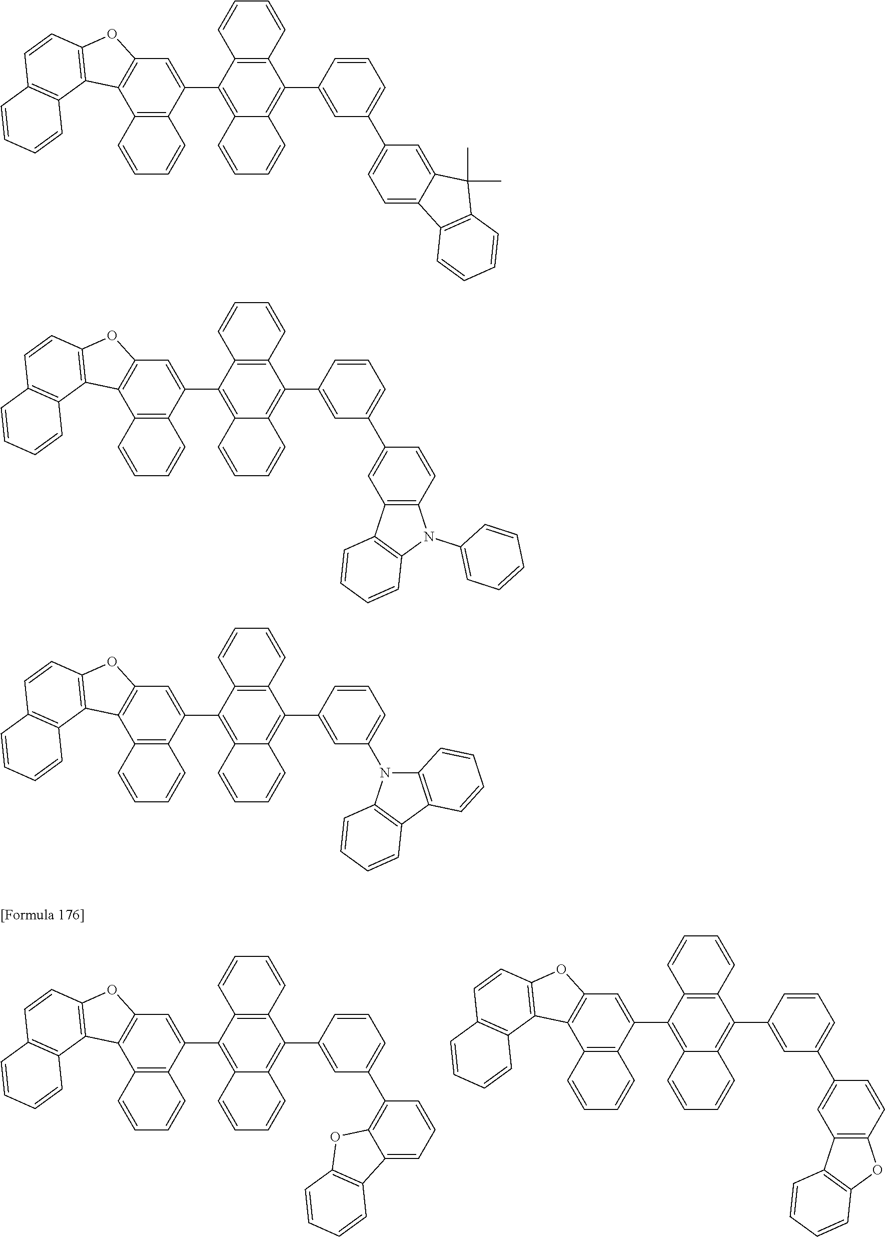

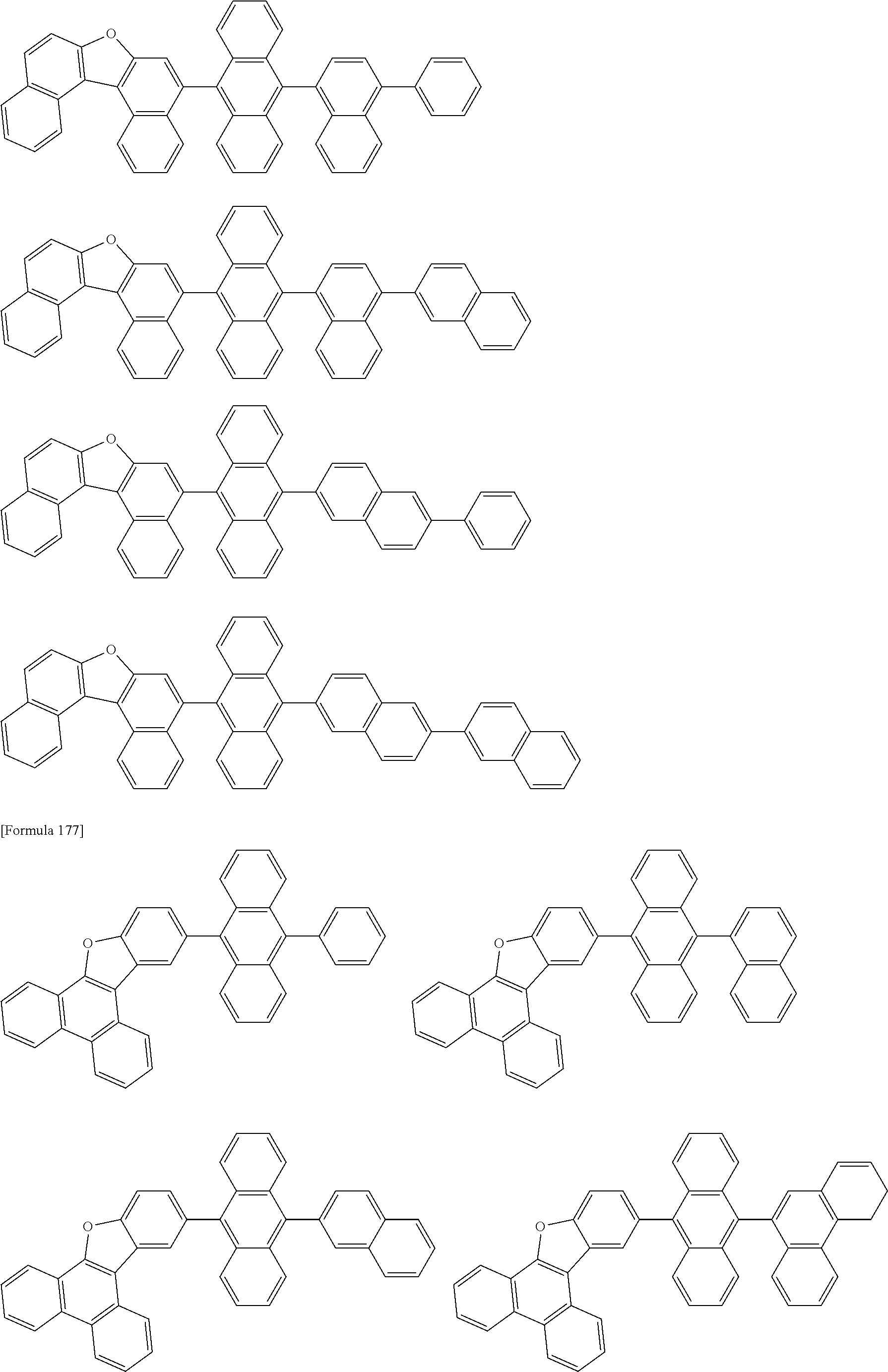

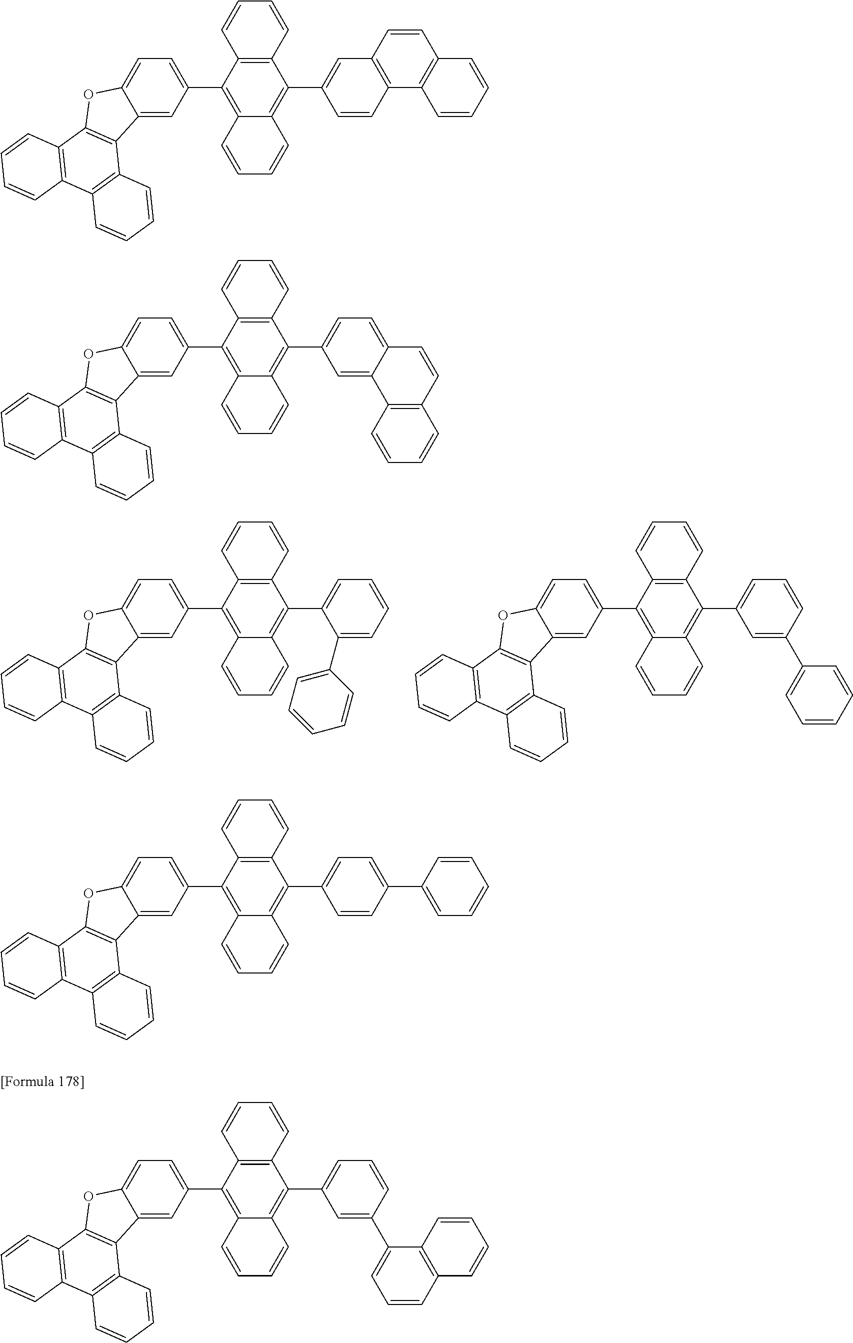 [SMILES: c1ccc2c(c1)ccc1cc(-c3c4ccccc4c(-c4ccc5oc6c7ccccc7c7ccccc7c6c5c4)c4ccccc34)ccc12 ; c1ccc2c(c1)ccc1ccc(-c3c4ccccc4c(-c4ccc5oc6c7ccccc7c7ccccc7c6c5c4)c4ccccc34)cc12 ; c1ccc(-c2ccccc2-c2c3ccccc3c(-c3ccc4oc5c6ccccc6c6ccccc6c5c4c3)c3ccccc23)cc1 ; c1ccc(-c2cccc(-c3c4ccccc4c(-c4ccc5oc6c7ccccc7c7ccccc7c6c5c4)c4ccccc34)c2)cc1 ; c1ccc(-c2ccc(-c3c4ccccc4c(-c4ccc5oc6c7ccccc7c7ccccc7c6c5c4)c4ccccc34)cc2)cc1 ; c1cc(-c2cccc3ccccc23)cc(-c2c3ccccc3c(-c3ccc4oc5c6ccccc6c6ccccc6c5c4c3)c3ccccc23)c1]

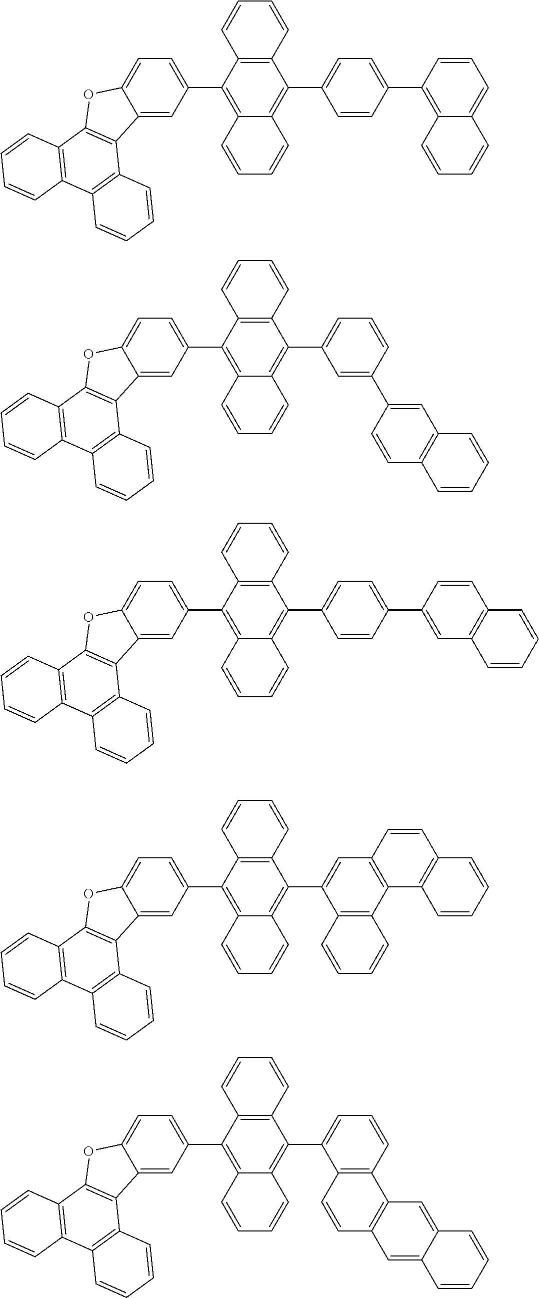 [SMILES: c1ccc2c(-c3ccc(-c4c5ccccc5c(-c5ccc6oc7c8ccccc8c8ccccc8c7c6c5)c5ccccc45)cc3)cccc2c1 ; c1cc(-c2ccc3ccccc3c2)cc(-c2c3ccccc3c(-c3ccc4oc5c6ccccc6c6ccccc6c5c4c3)c3ccccc23)c1 ; c1ccc2cc(-c3ccc(-c4c5ccccc5c(-c5ccc6oc7c8ccccc8c8ccccc8c7c6c5)c5ccccc45)cc3)ccc2c1 ; c1ccc2c(c1)ccc1cc(-c3c4ccccc4c(-c4ccc5oc6c7ccccc7c7ccccc7c6c5c4)c4ccccc34)c3ccccc3c12 ; c1ccc2cc3c(ccc4c(-c5c6ccccc6c(-c6ccc7oc8c9ccccc9c9ccccc9c8c7c6)c6ccccc56)cccc43)cc2c1]

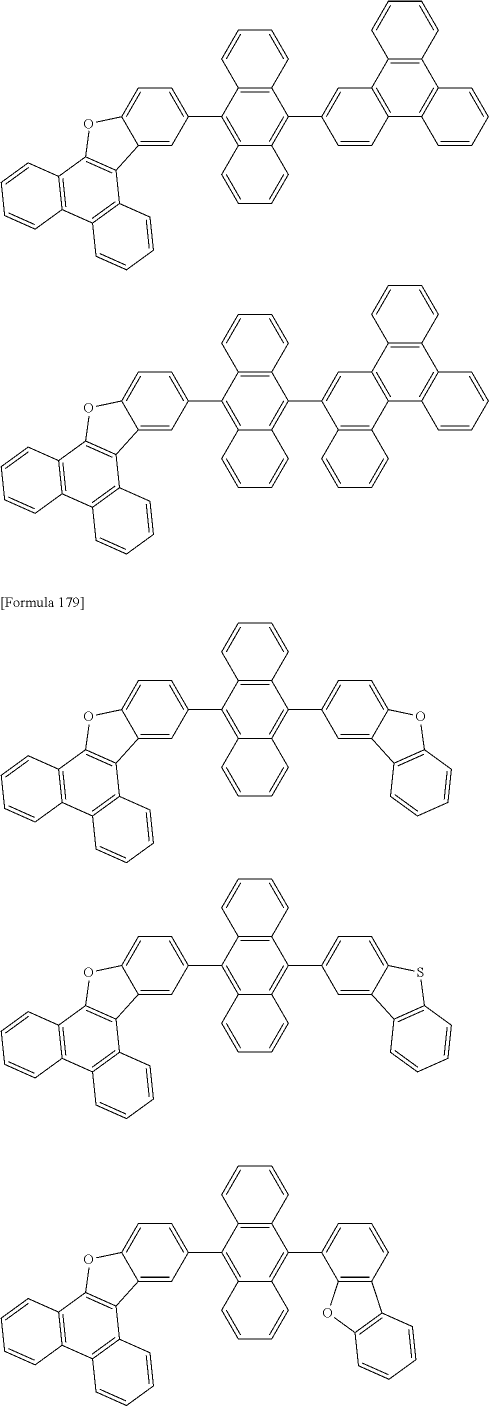 [SMILES: c1ccc2c(-c3ccc4oc5c6ccccc6c6ccccc6c5c4c3)c3ccccc3c(-c3ccc4c5ccccc5c5ccccc5c4c3)c2c1 ; c1ccc2c(-c3cc4c5ccccc5c5ccccc5c4c4ccccc34)c3ccccc3c(-c3ccc4oc5c6ccccc6c6ccccc6c5c4c3)c2c1 ; c1ccc2c(c1)oc1ccc(-c3c4ccccc4c(-c4ccc5oc6c7ccccc7c7ccccc7c6c5c4)c4ccccc34)cc12 ; c1ccc2c(c1)sc1ccc(-c3c4ccccc4c(-c4ccc5oc6c7ccccc7c7ccccc7c6c5c4)c4ccccc34)cc12 ; c1ccc2c(c1)oc1c(-c3c4ccccc4c(-c4ccc5oc6c7ccccc7c7ccccc7c6c5c4)c4ccccc34)cccc12]

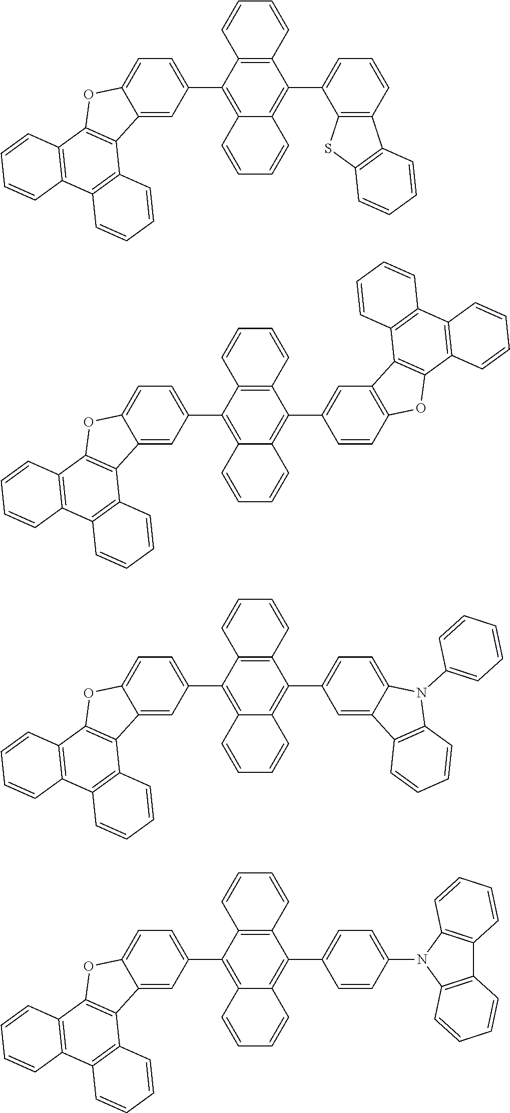 [SMILES: c1ccc2c(c1)sc1c(-c3c4ccccc4c(-c4ccc5oc6c7ccccc7c7ccccc7c6c5c4)c4ccccc34)cccc12 ; c1ccc2c(-c3ccc4oc5c6ccccc6c6ccccc6c5c4c3)c3ccccc3c(-c3ccc4oc5c6ccccc6c6ccccc6c5c4c3)c2c1 ; c1ccc(-n2c3ccccc3c3cc(-c4c5ccccc5c(-c5ccc6oc7c8ccccc8c8ccccc8c7c6c5)c5ccccc45)ccc32)cc1 ; c1ccc2c(-c3ccc4oc5c6ccccc6c6ccccc6c5c4c3)c3ccccc3c(-c3ccc(-n4c5ccccc5c5ccccc54)cc3)c2c1]

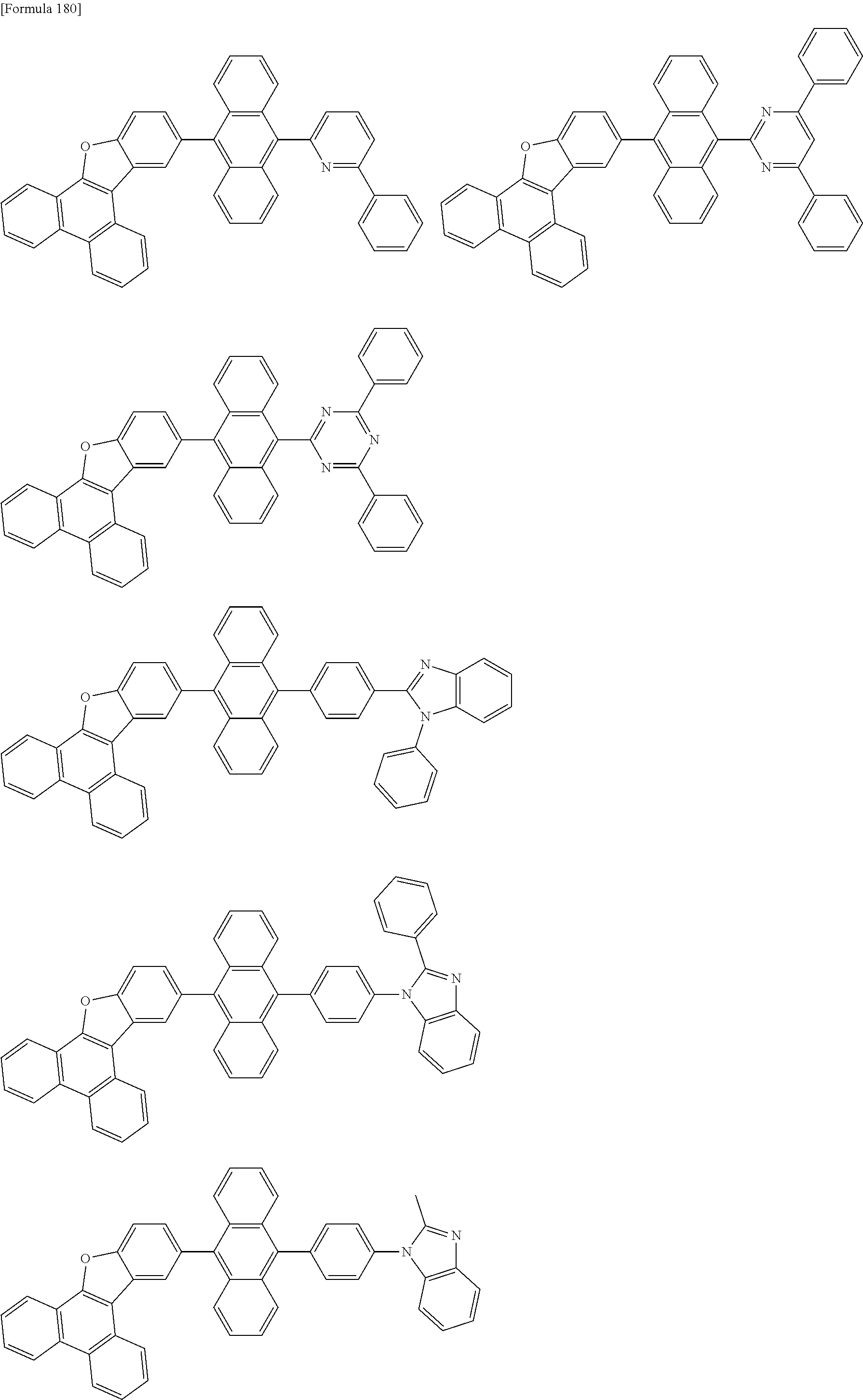

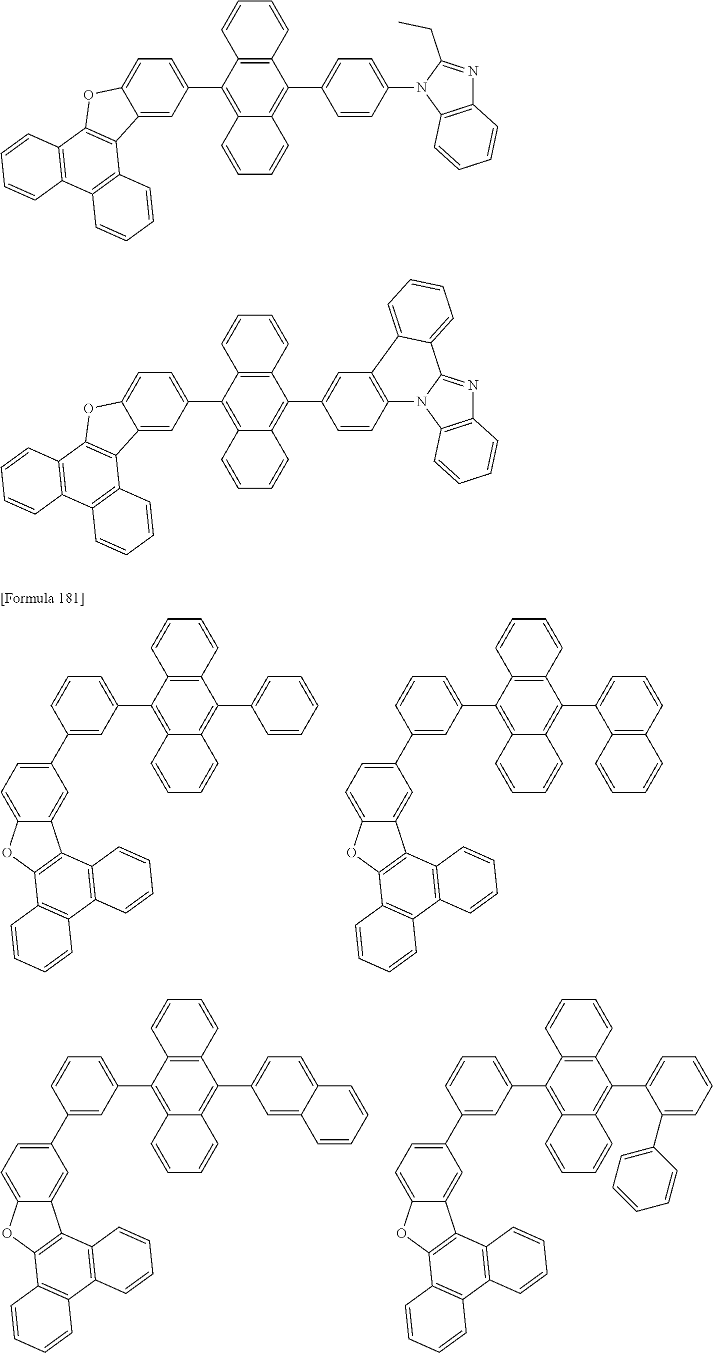

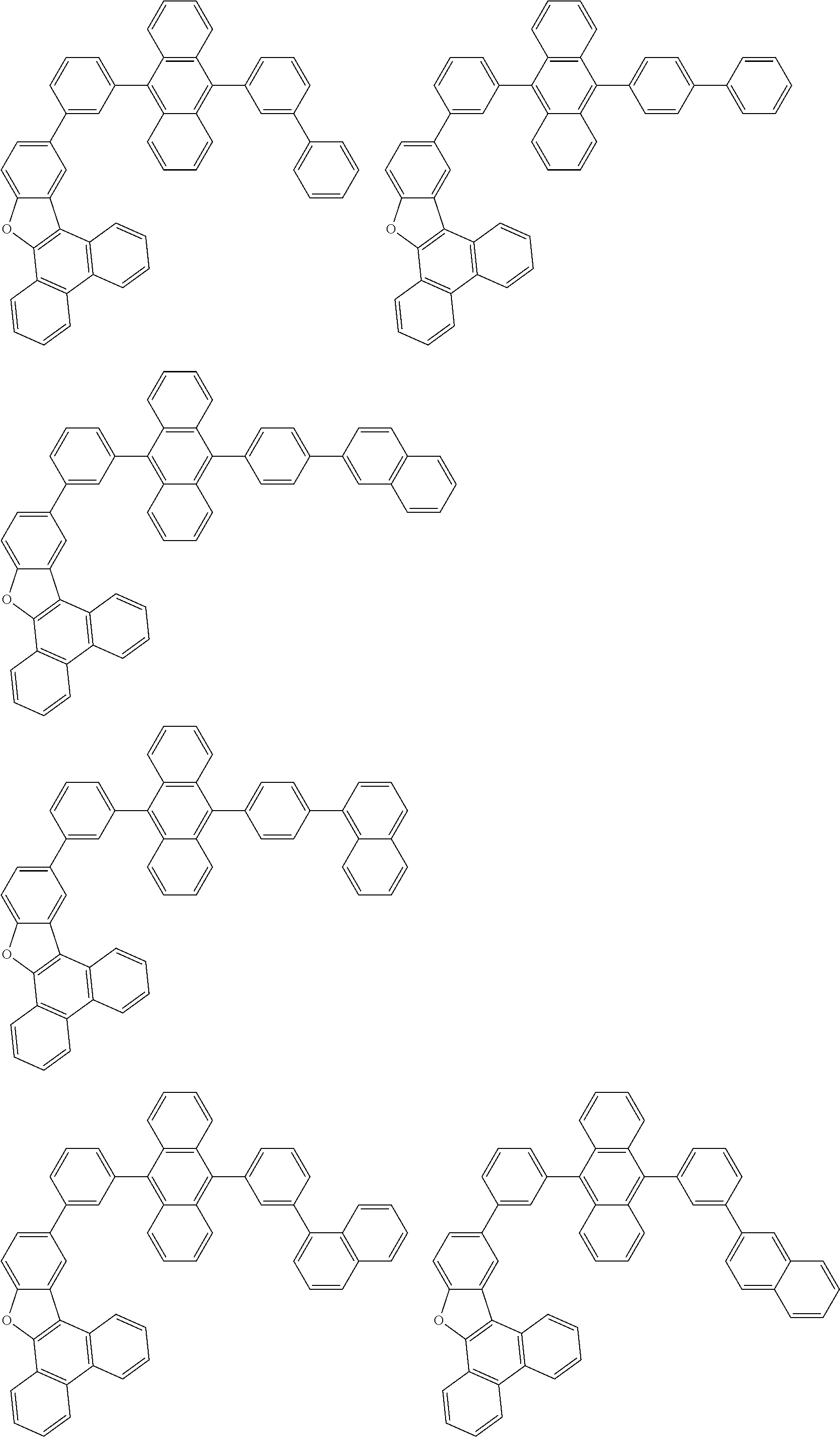

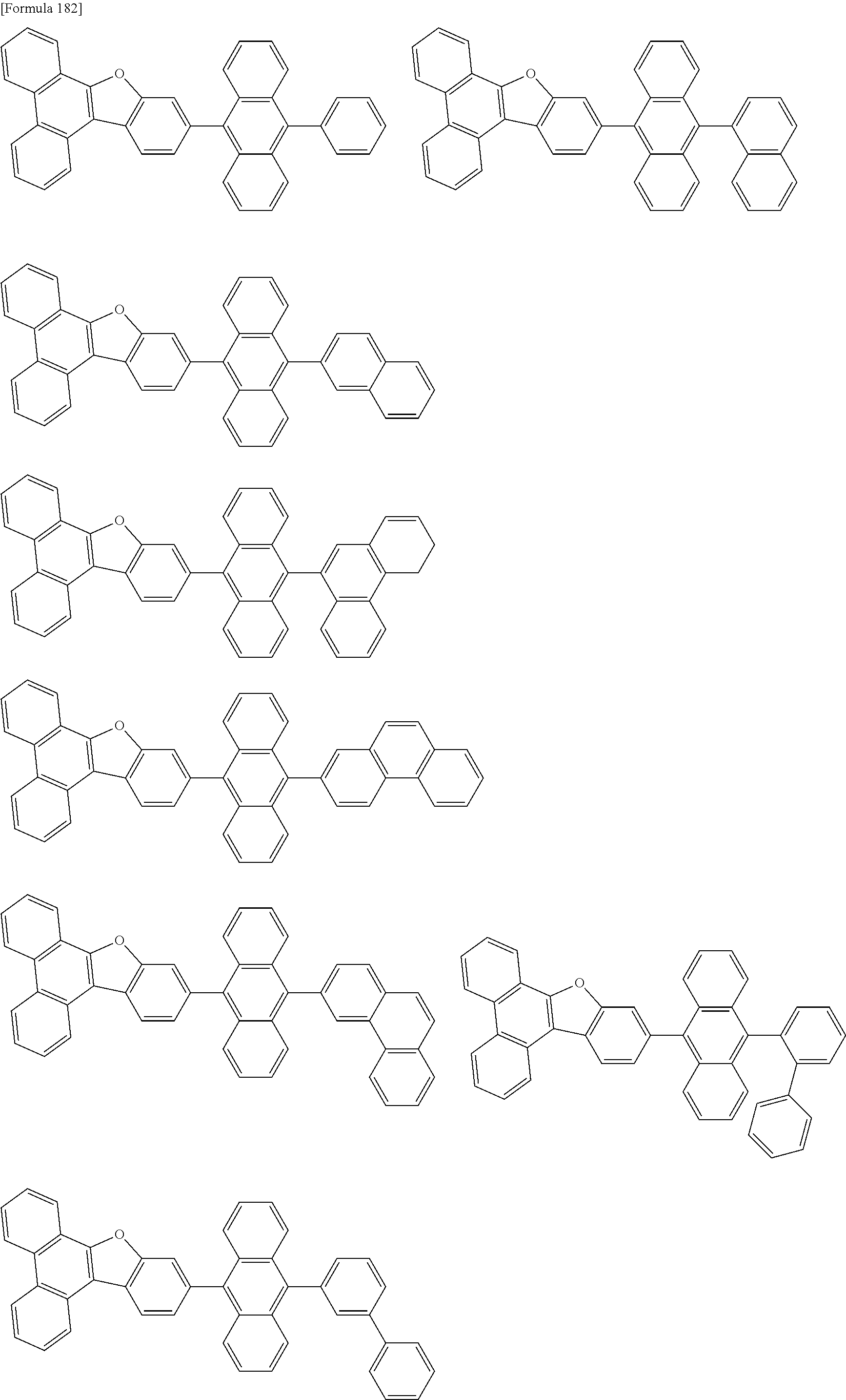

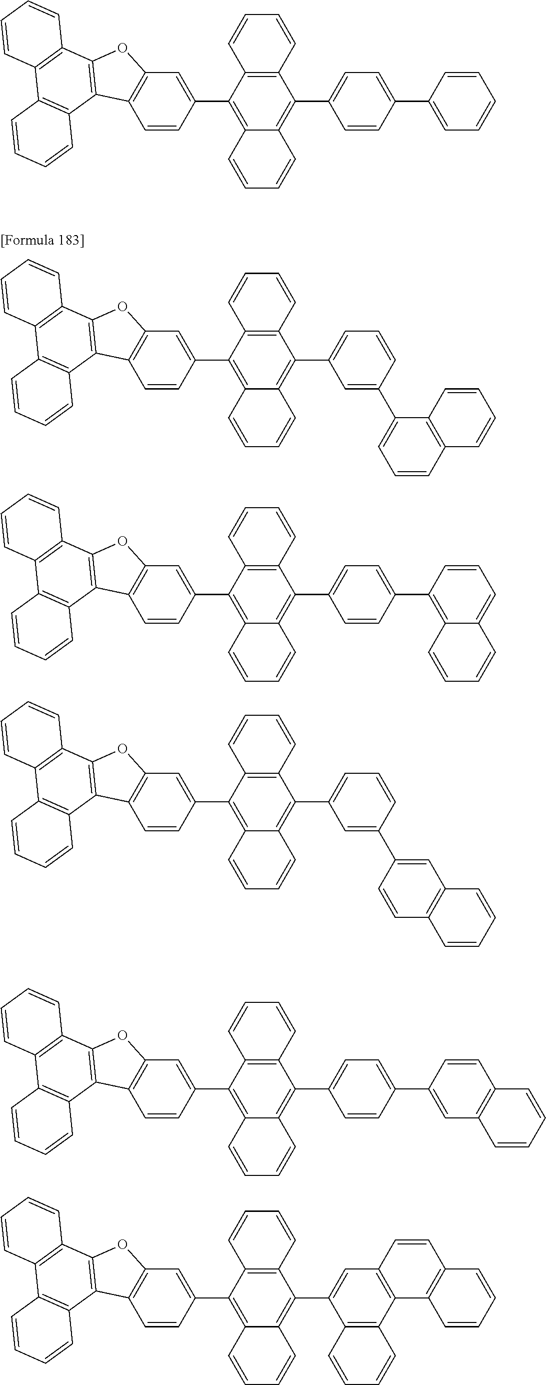

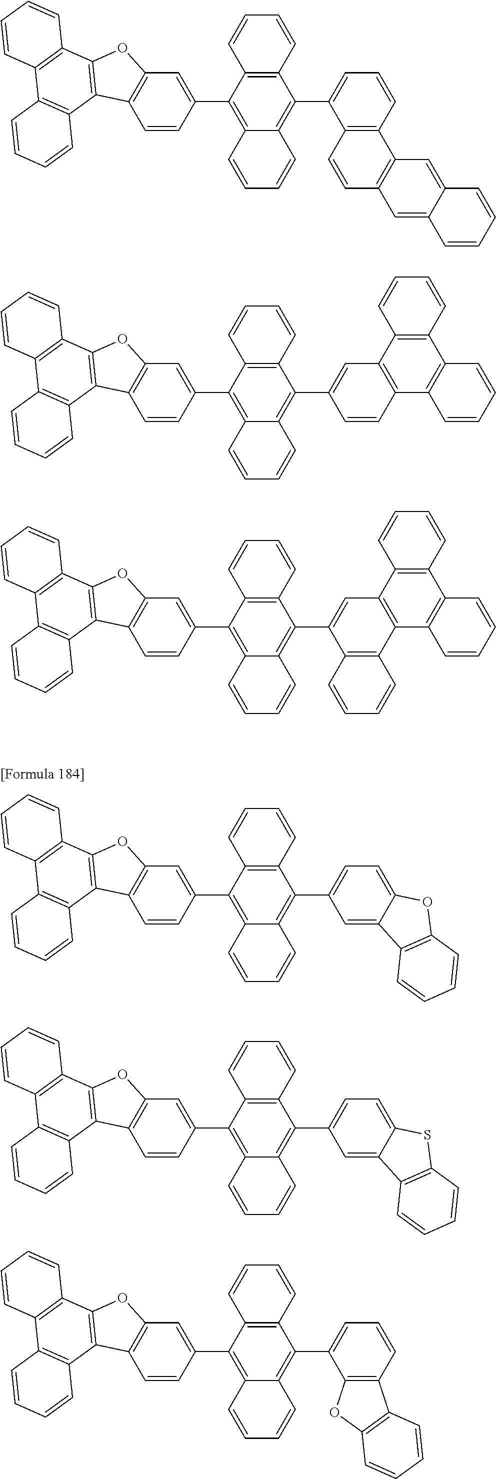

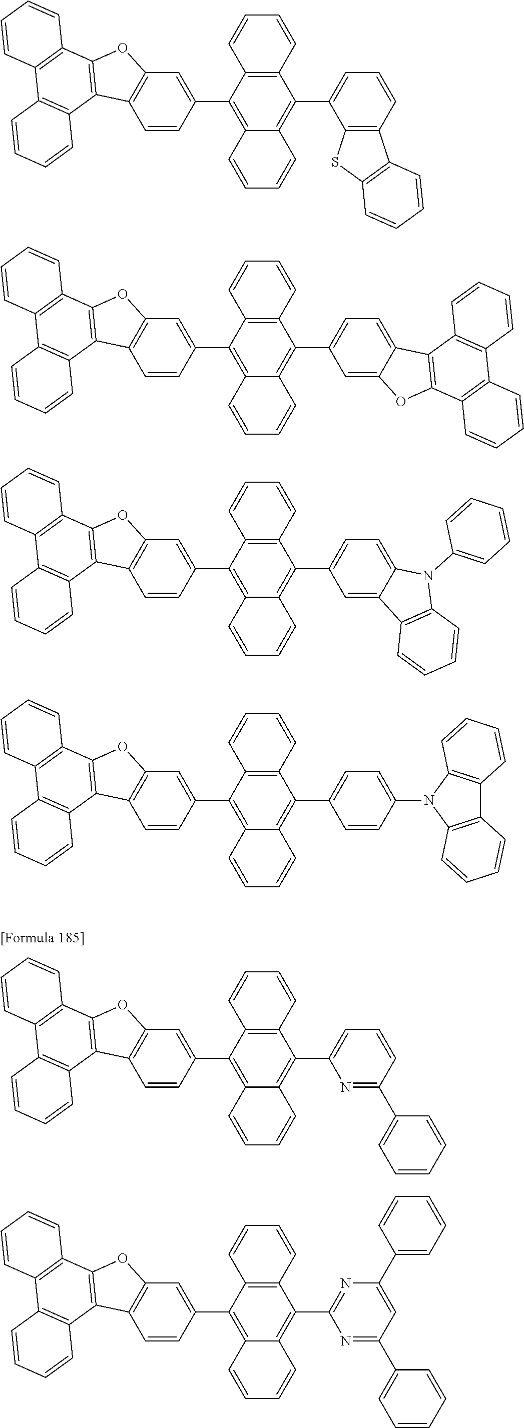

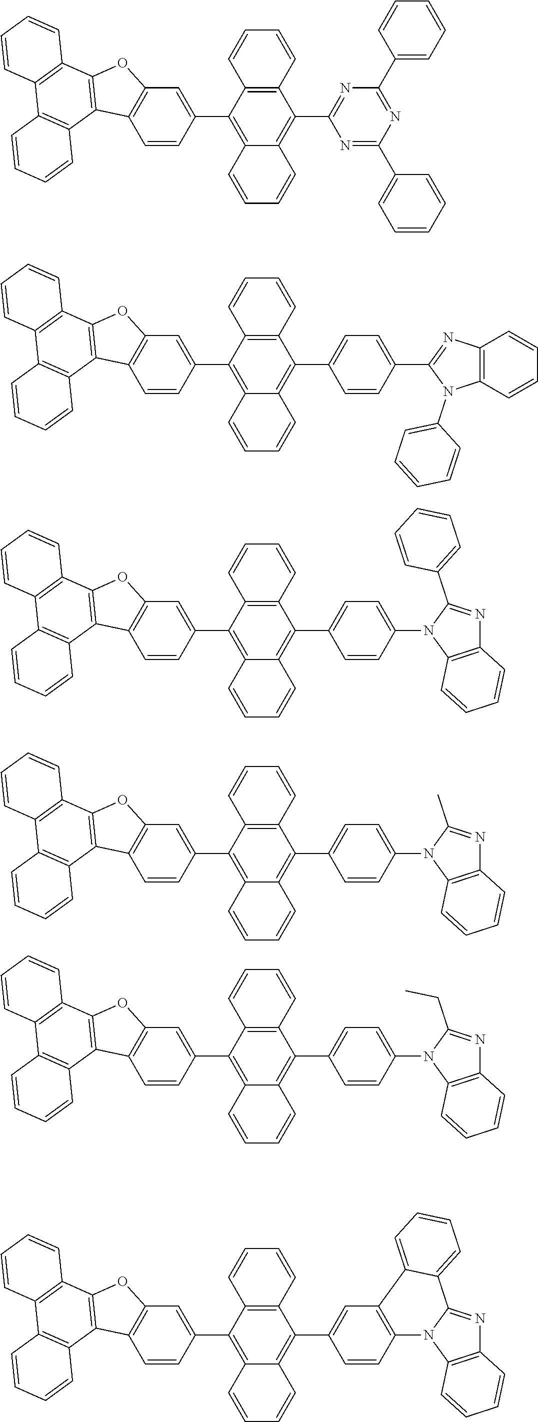

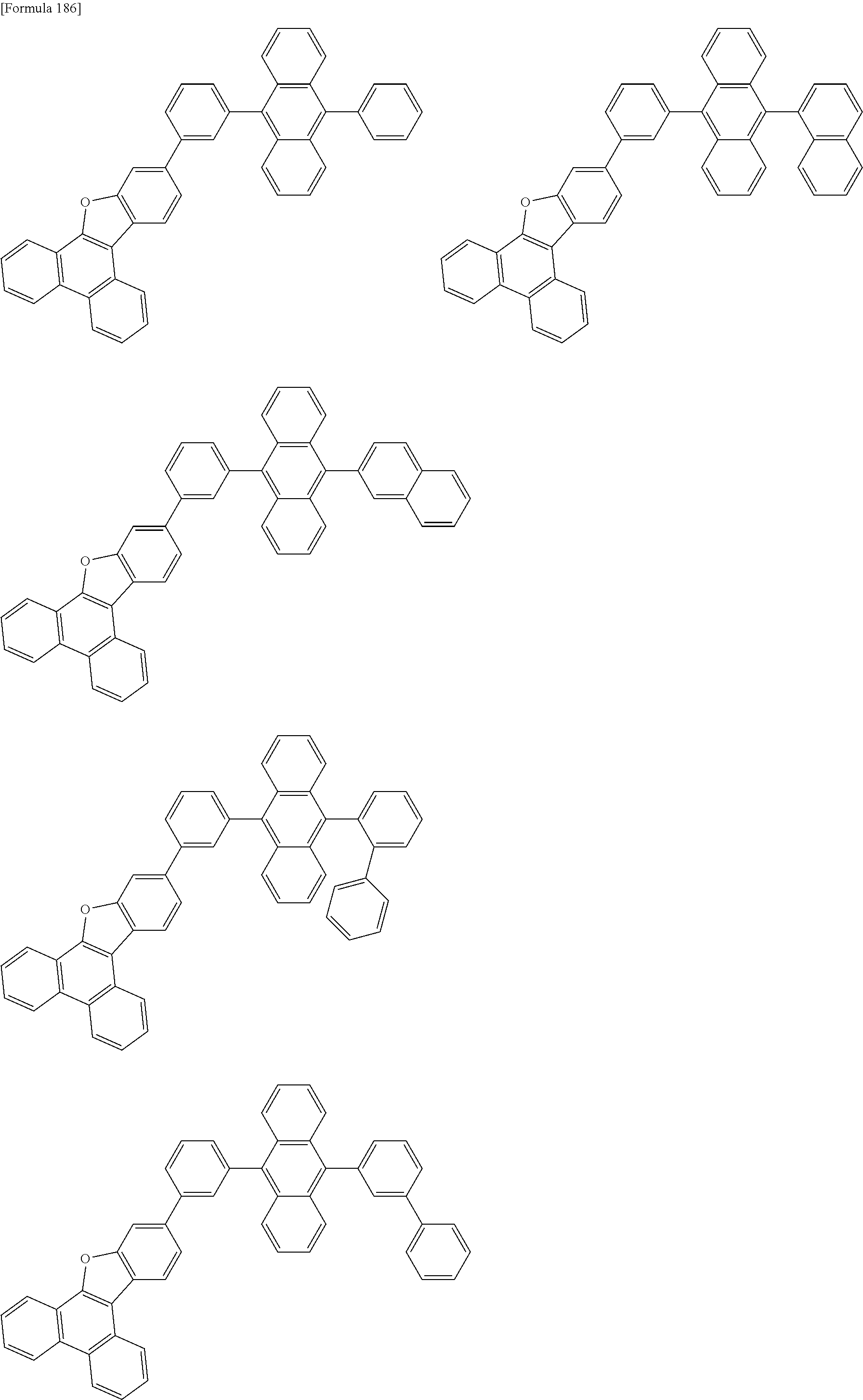 [SMILES: c1ccc(-c2c3ccccc3c(-c3cccc(-c4ccc5c(c4)oc4c6ccccc6c6ccccc6c54)c3)c3ccccc23)cc1 ; c1cc(-c2ccc3c(c2)oc2c4ccccc4c4ccccc4c32)cc(-c2c3ccccc3c(-c3cccc4ccccc34)c3ccccc23)c1 ; c1cc(-c2ccc3c(c2)oc2c4ccccc4c4ccccc4c32)cc(-c2c3ccccc3c(-c3ccc4ccccc4c3)c3ccccc23)c1 ; c1ccc(-c2ccccc2-c2c3ccccc3c(-c3cccc(-c4ccc5c(c4)oc4c6ccccc6c6ccccc6c54)c3)c3ccccc23)cc1 ; c1ccc(-c2cccc(-c3c4ccccc4c(-c4cccc(-c5ccc6c(c5)oc5c7ccccc7c7ccccc7c65)c4)c4ccccc34)c2)cc1]

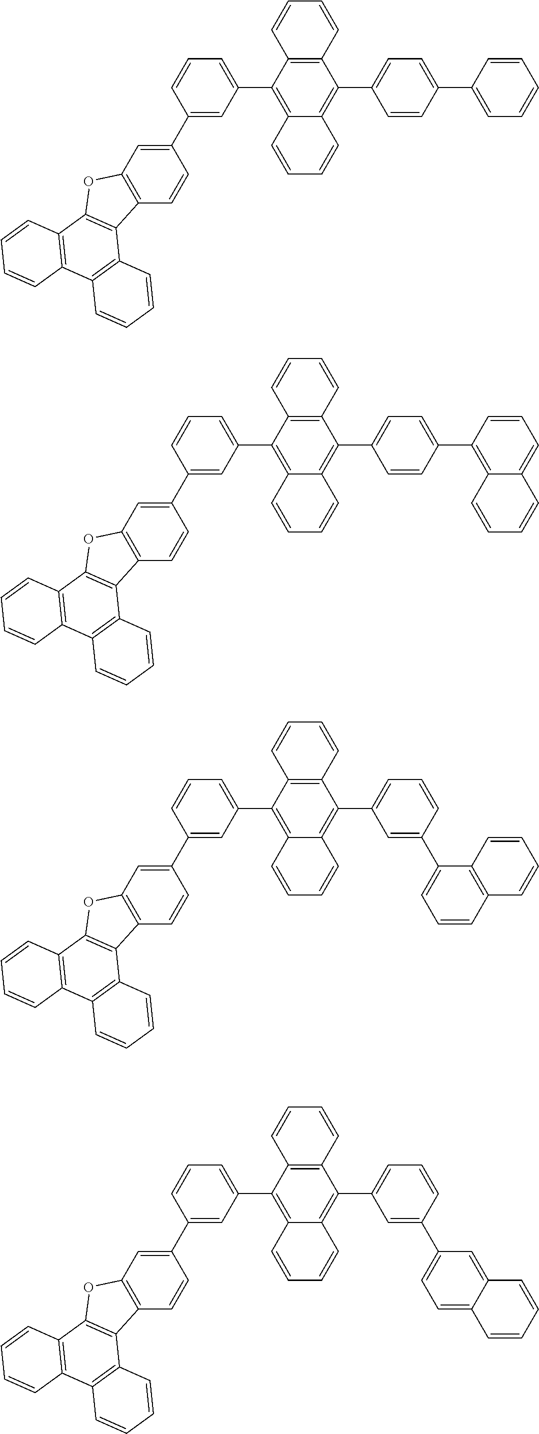 [SMILES: c1ccc(-c2ccc(-c3c4ccccc4c(-c4cccc(-c5ccc6c(c5)oc5c7ccccc7c7ccccc7c65)c4)c4ccccc34)cc2)cc1 ; c1cc(-c2ccc3c(c2)oc2c4ccccc4c4ccccc4c32)cc(-c2c3ccccc3c(-c3ccc(-c4cccc5ccccc45)cc3)c3ccccc23)c1 ; c1cc(-c2ccc3c(c2)oc2c4ccccc4c4ccccc4c32)cc(-c2c3ccccc3c(-c3cccc(-c4cccc5ccccc45)c3)c3ccccc23)c1 ; c1cc(-c2ccc3ccccc3c2)cc(-c2c3ccccc3c(-c3cccc(-c4ccc5c(c4)oc4c6ccccc6c6ccccc6c54)c3)c3ccccc23)c1]

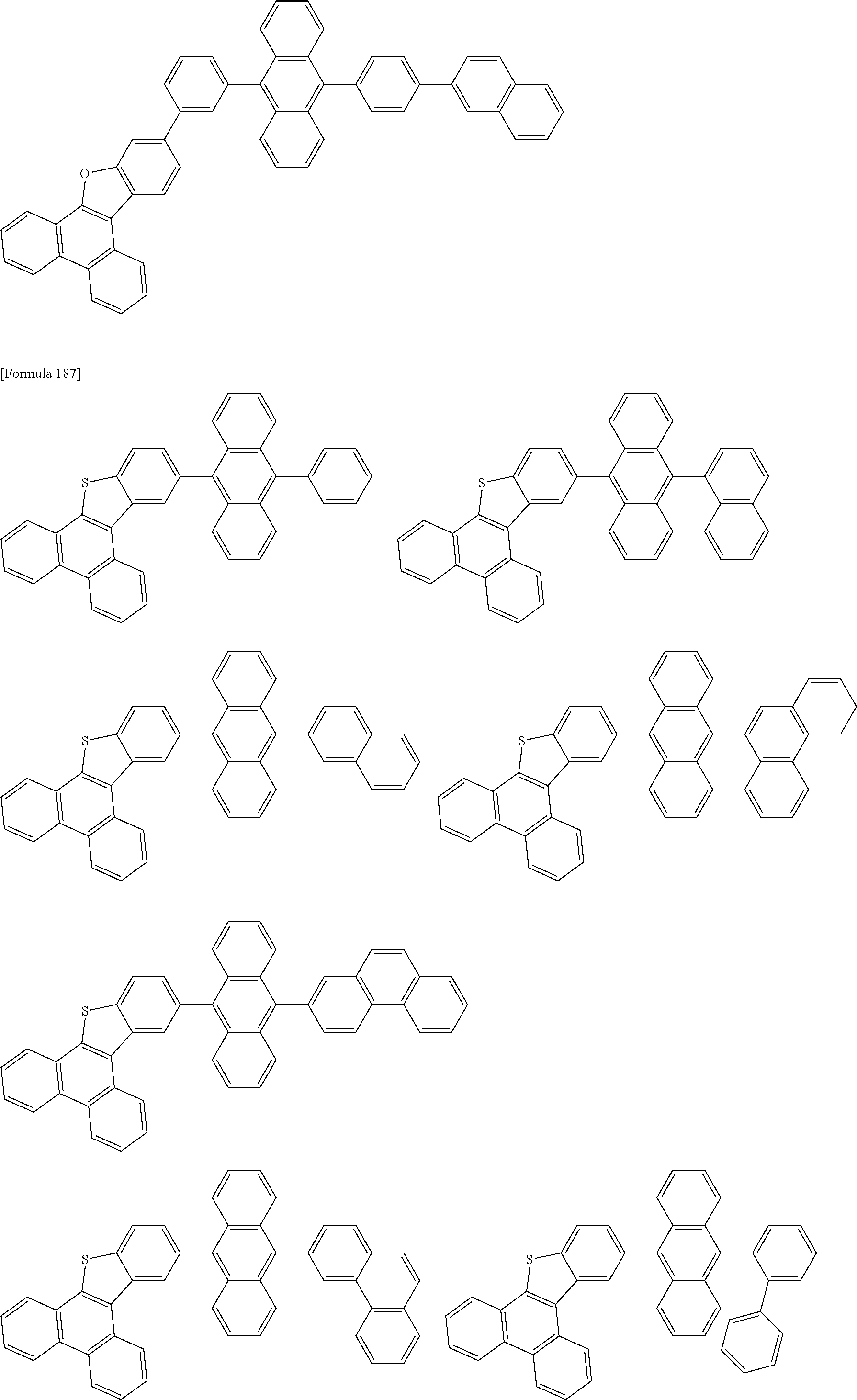

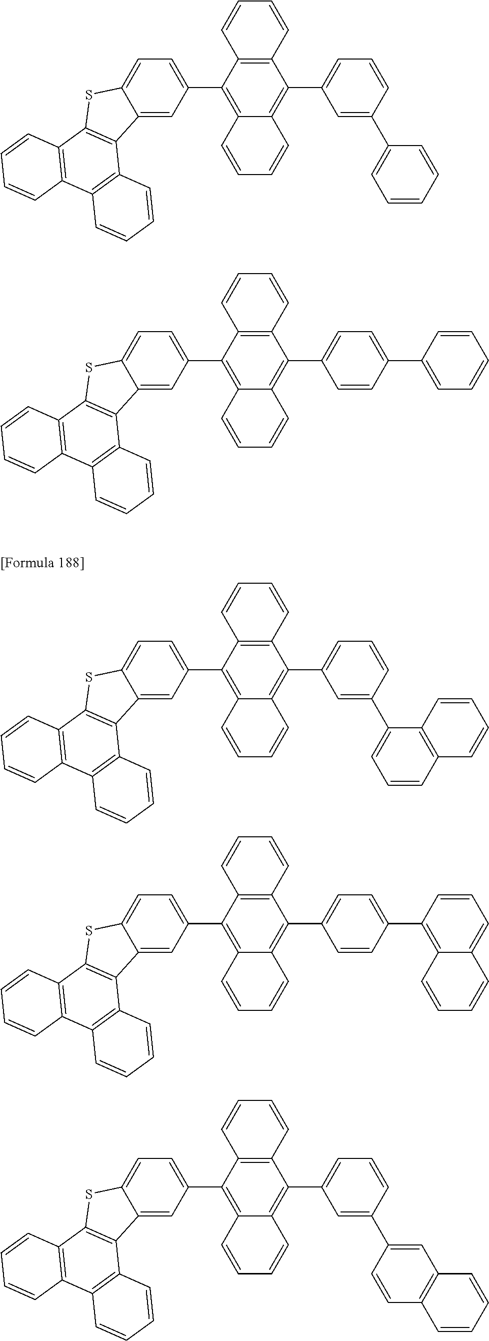 [SMILES: c1ccc(-c2cccc(-c3c4ccccc4c(-c4ccc5sc6c7ccccc7c7ccccc7c6c5c4)c4ccccc34)c2)cc1 ; c1ccc(-c2ccc(-c3c4ccccc4c(-c4ccc5sc6c7ccccc7c7ccccc7c6c5c4)c4ccccc34)cc2)cc1 ; c1cc(-c2cccc3ccccc23)cc(-c2c3ccccc3c(-c3ccc4sc5c6ccccc6c6ccccc6c5c4c3)c3ccccc23)c1 ; c1ccc2c(-c3ccc(-c4c5ccccc5c(-c5ccc6sc7c8ccccc8c8ccccc8c7c6c5)c5ccccc45)cc3)cccc2c1 ; c1cc(-c2ccc3ccccc3c2)cc(-c2c3ccccc3c(-c3ccc4sc5c6ccccc6c6ccccc6c5c4c3)c3ccccc23)c1]

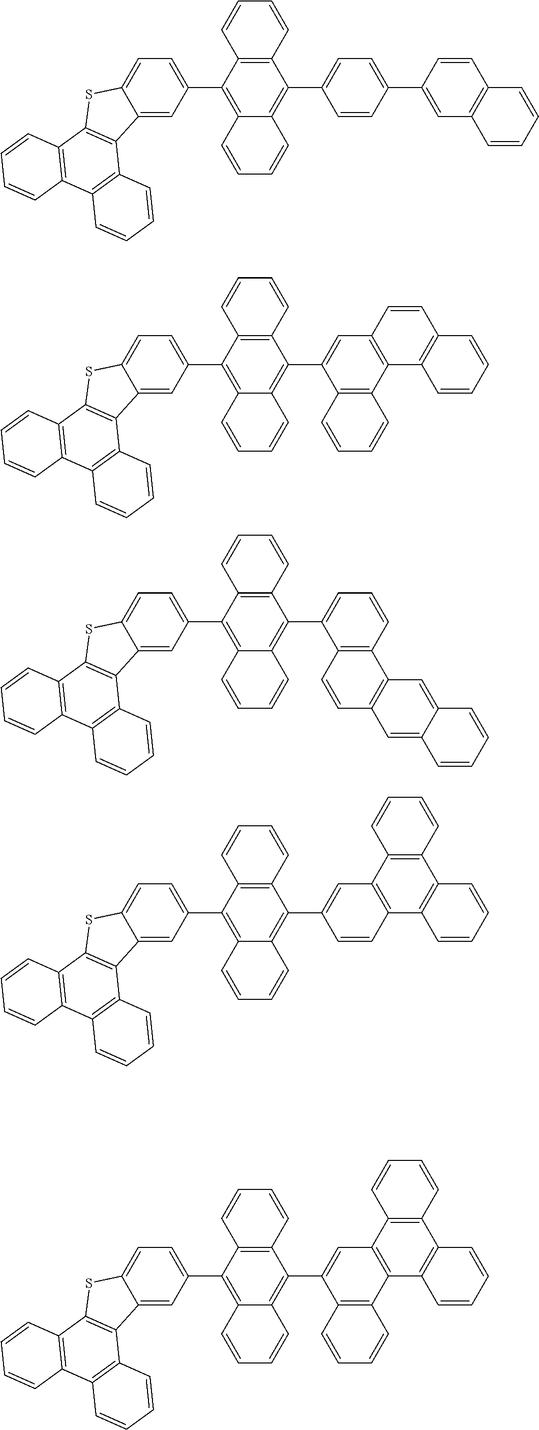

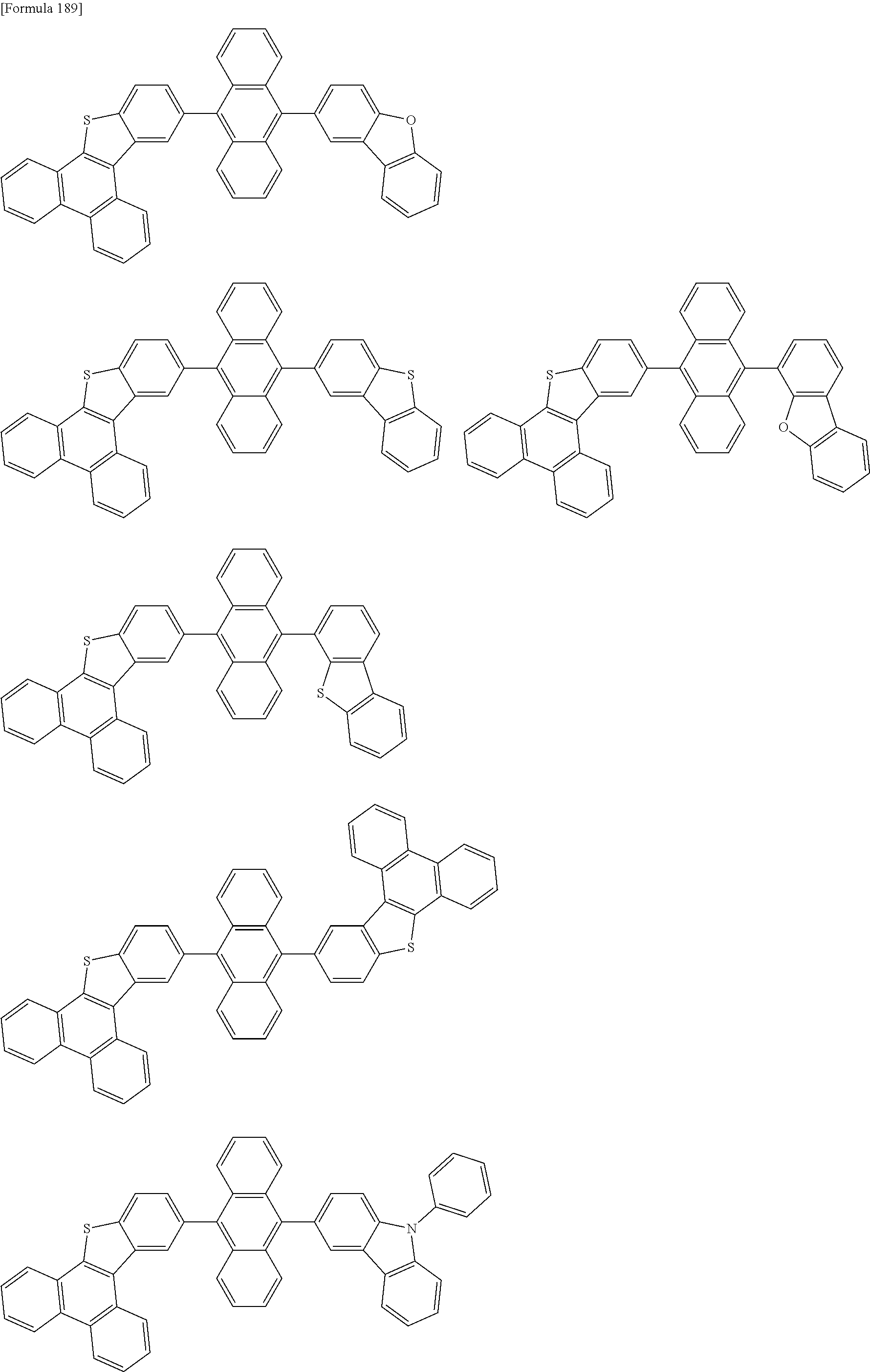

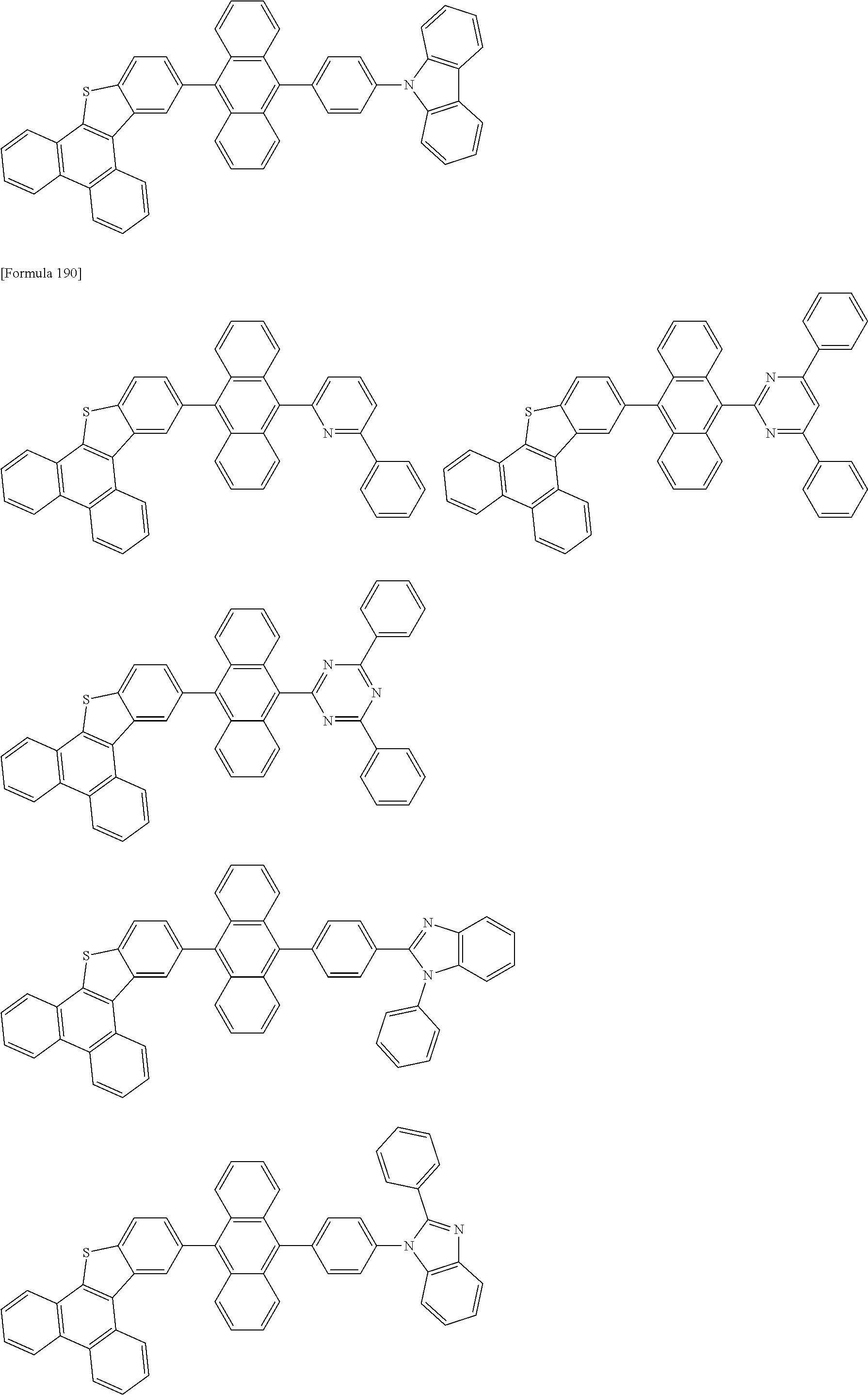

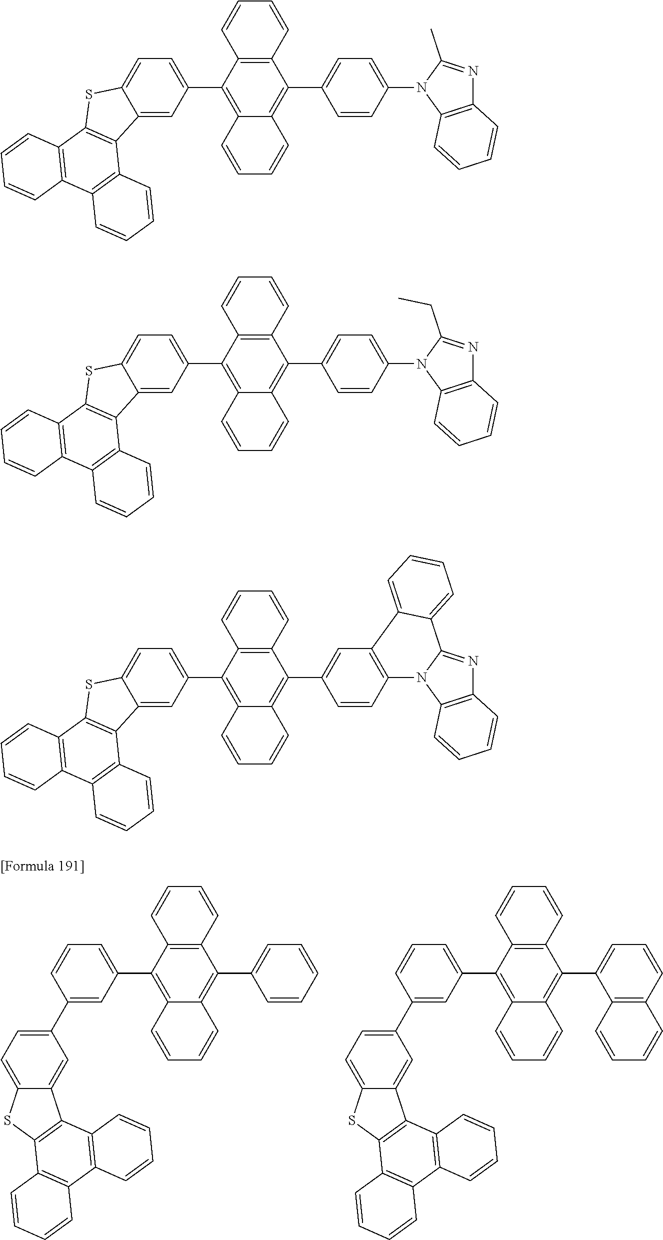 [SMILES: Cc1nc2ccccc2n1-c1ccc(-c2c3ccccc3c(-c3ccc4sc5c6ccccc6c6ccccc6c5c4c3)c3ccccc23)cc1 ; CCc1nc2ccccc2n1-c1ccc(-c2c3ccccc3c(-c3ccc4sc5c6ccccc6c6ccccc6c5c4c3)c3ccccc23)cc1 ; c1ccc2c(c1)nc1c3ccccc3c3cc(-c4c5ccccc5c(-c5ccc6sc7c8ccccc8c8ccccc8c7c6c5)c5ccccc45)ccc3n21 ; c1ccc(-c2c3ccccc3c(-c3cccc(-c4ccc5sc6c7ccccc7c7ccccc7c6c5c4)c3)c3ccccc23)cc1 ; c1cc(-c2ccc3sc4c5ccccc5c5ccccc5c4c3c2)cc(-c2c3ccccc3c(-c3cccc4ccccc34)c3ccccc23)c1]

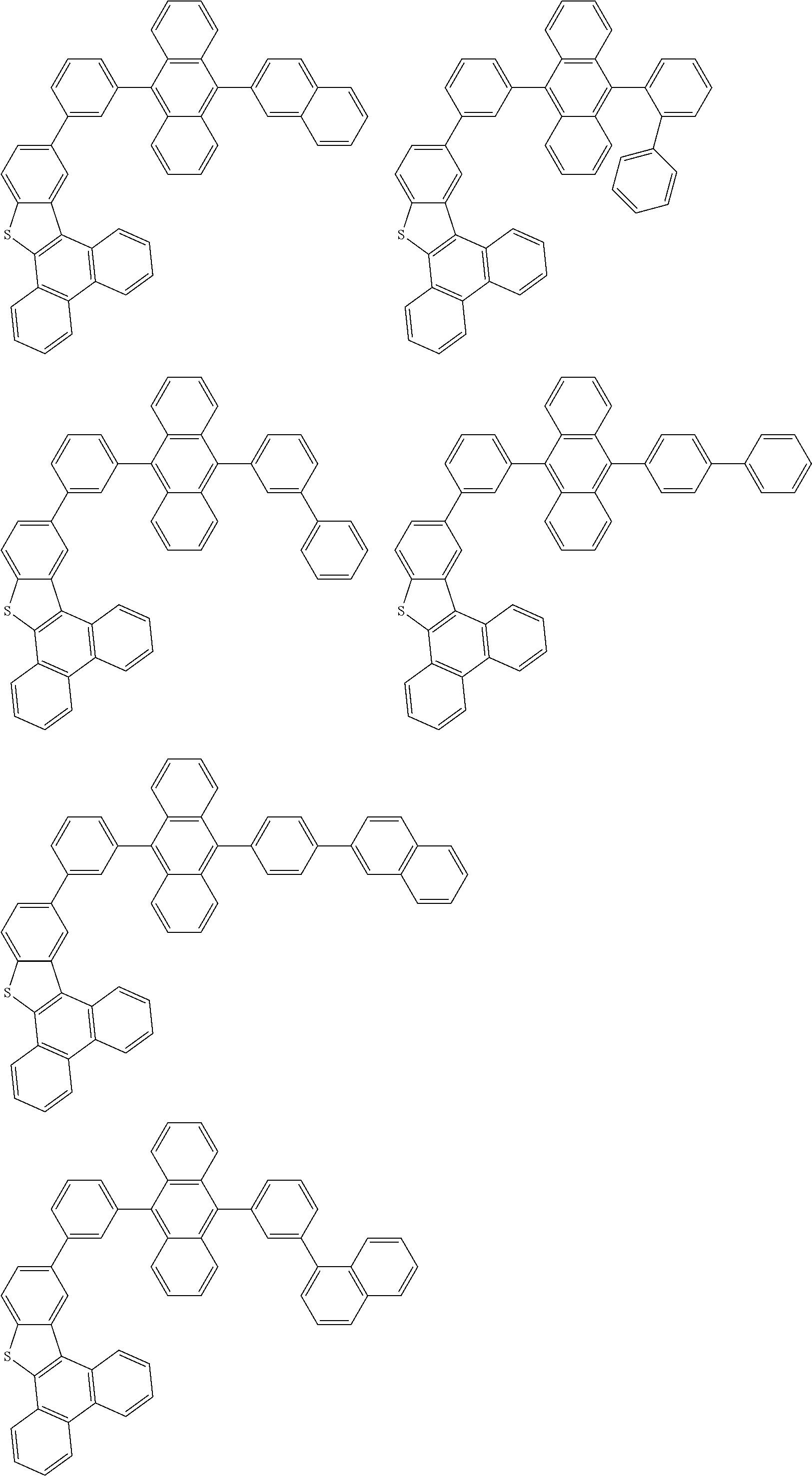

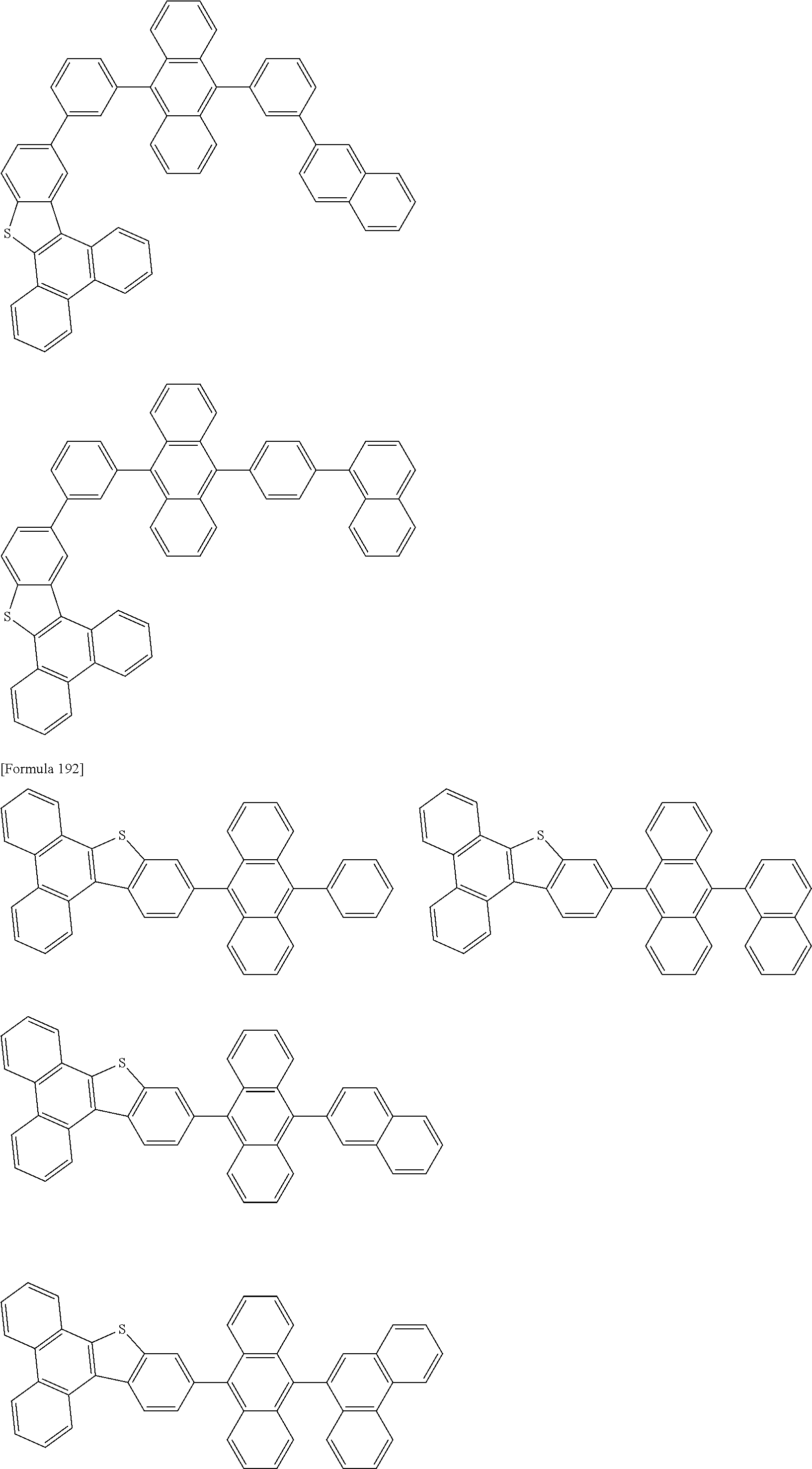

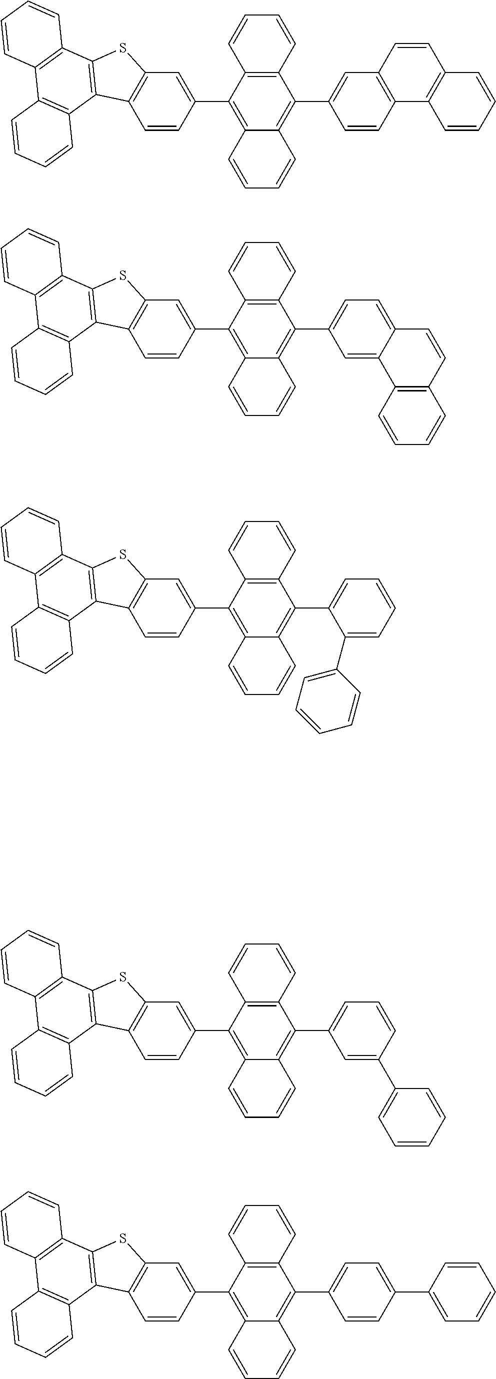 [SMILES: c1ccc2c(c1)ccc1cc(-c3c4ccccc4c(-c4ccc5c(c4)sc4c6ccccc6c6ccccc6c54)c4ccccc34)ccc12 ; c1ccc2c(c1)ccc1ccc(-c3c4ccccc4c(-c4ccc5c(c4)sc4c6ccccc6c6ccccc6c54)c4ccccc34)cc12 ; c1ccc(-c2ccccc2-c2c3ccccc3c(-c3ccc4c(c3)sc3c5ccccc5c5ccccc5c43)c3ccccc23)cc1 ; c1ccc(-c2cccc(-c3c4ccccc4c(-c4ccc5c(c4)sc4c6ccccc6c6ccccc6c54)c4ccccc34)c2)cc1 ; c1ccc(-c2ccc(-c3c4ccccc4c(-c4ccc5c(c4)sc4c6ccccc6c6ccccc6c54)c4ccccc34)cc2)cc1]

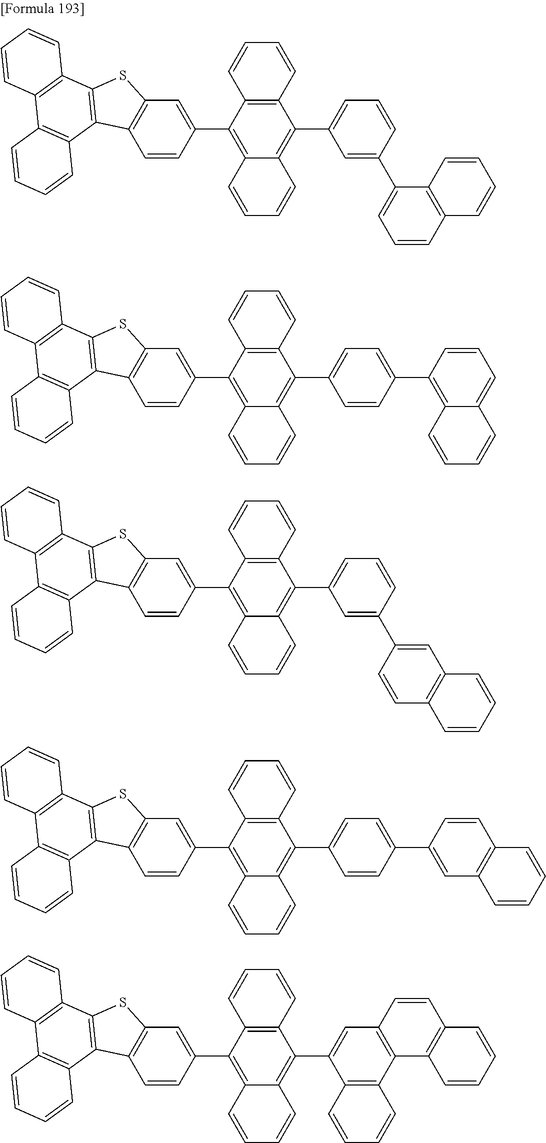

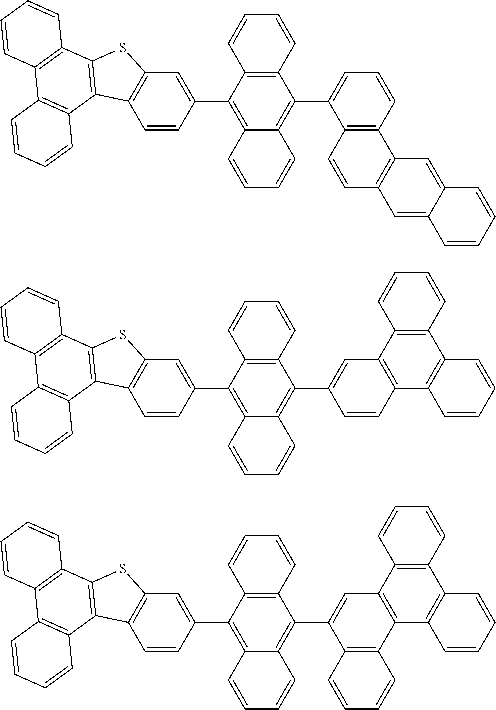

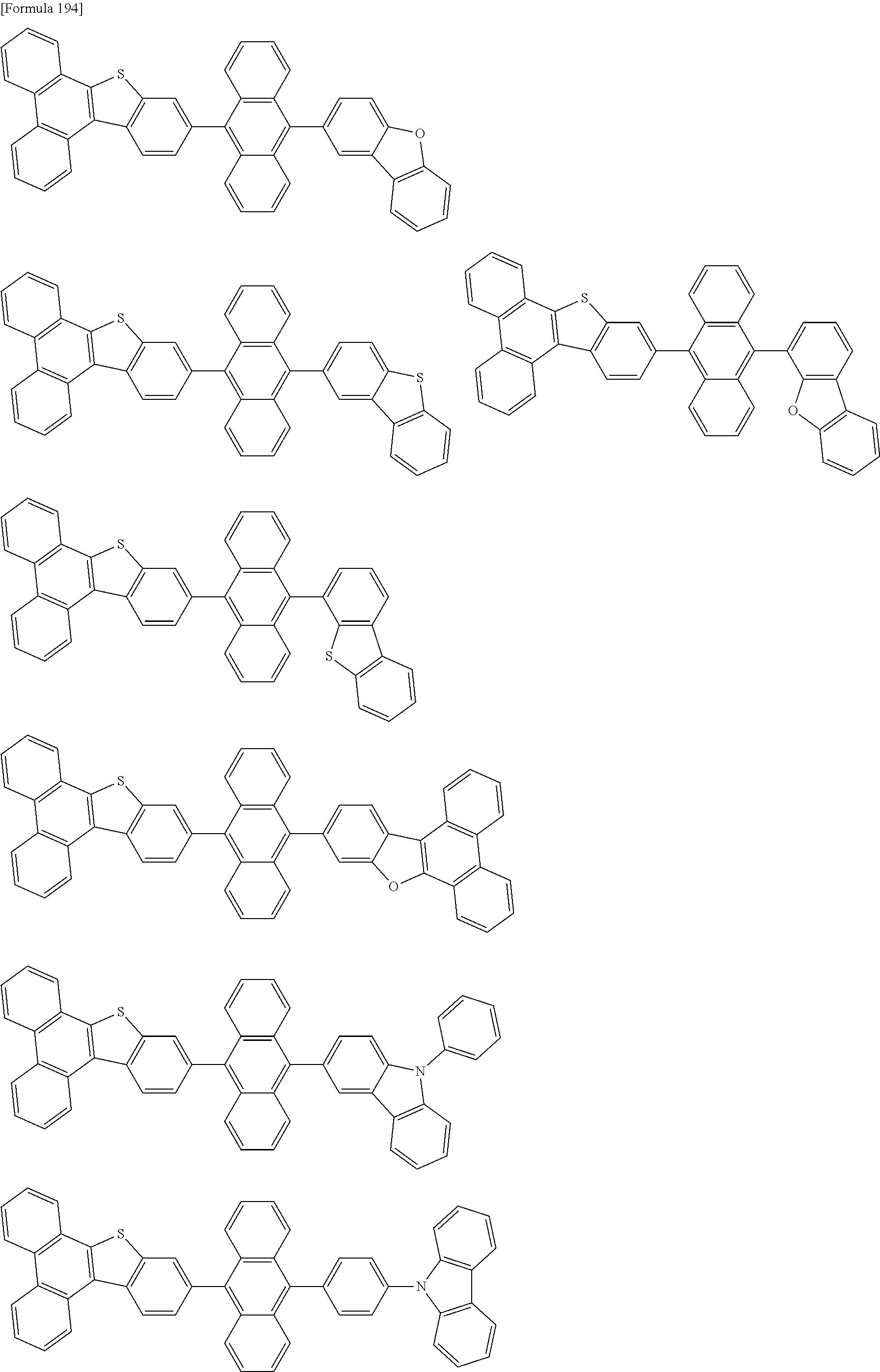

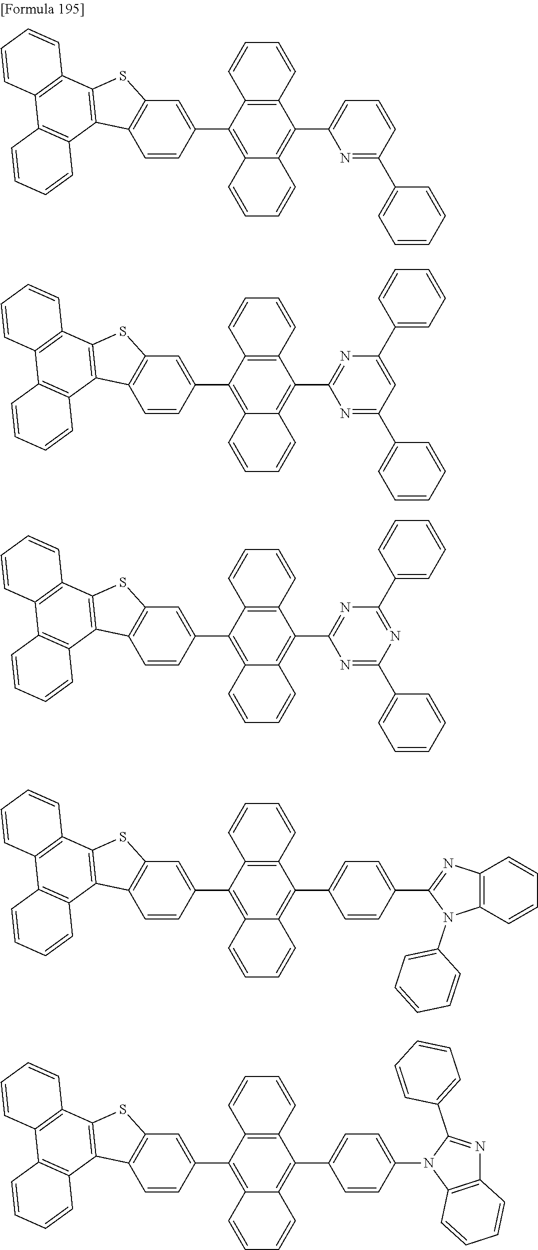

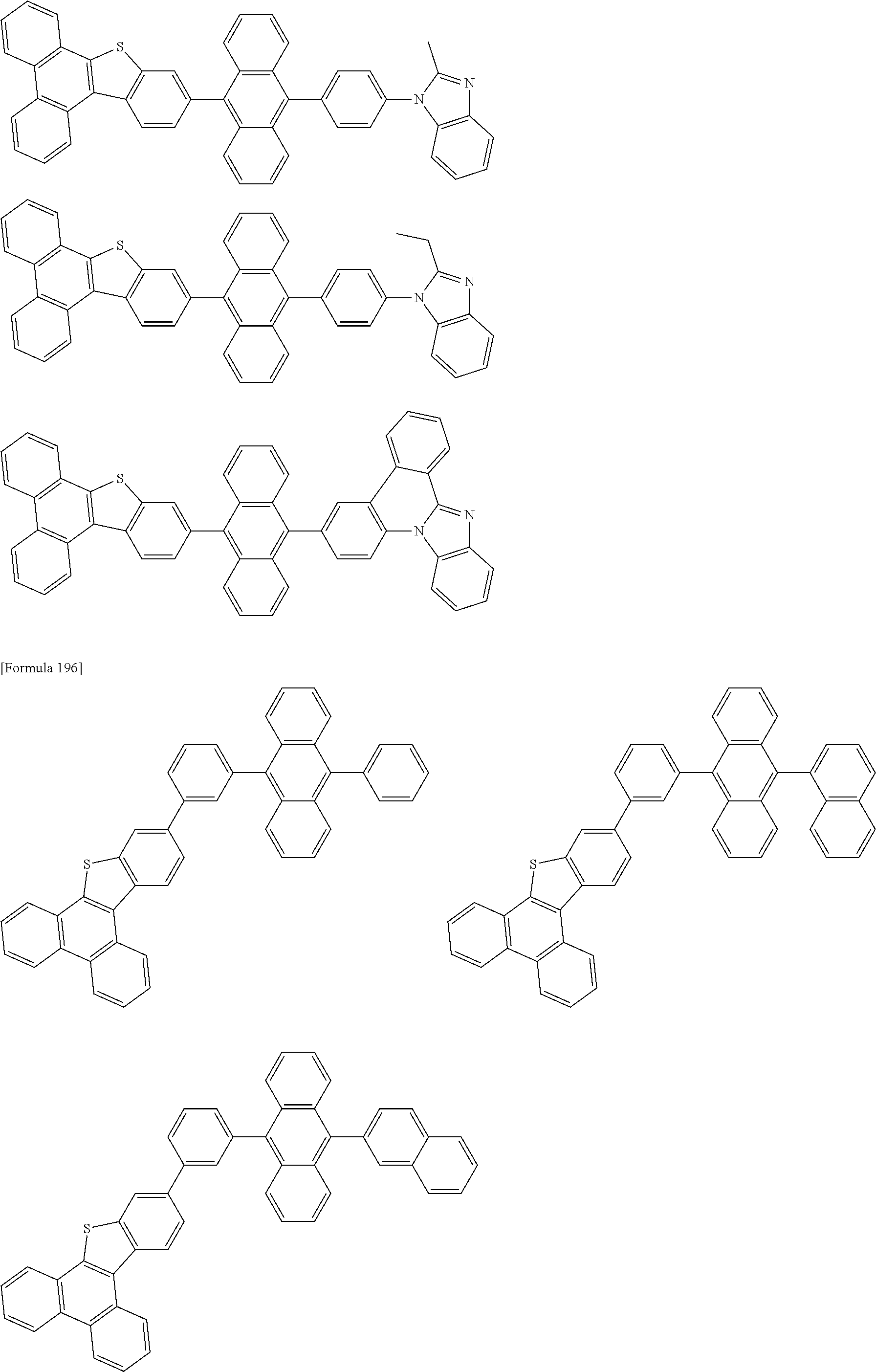

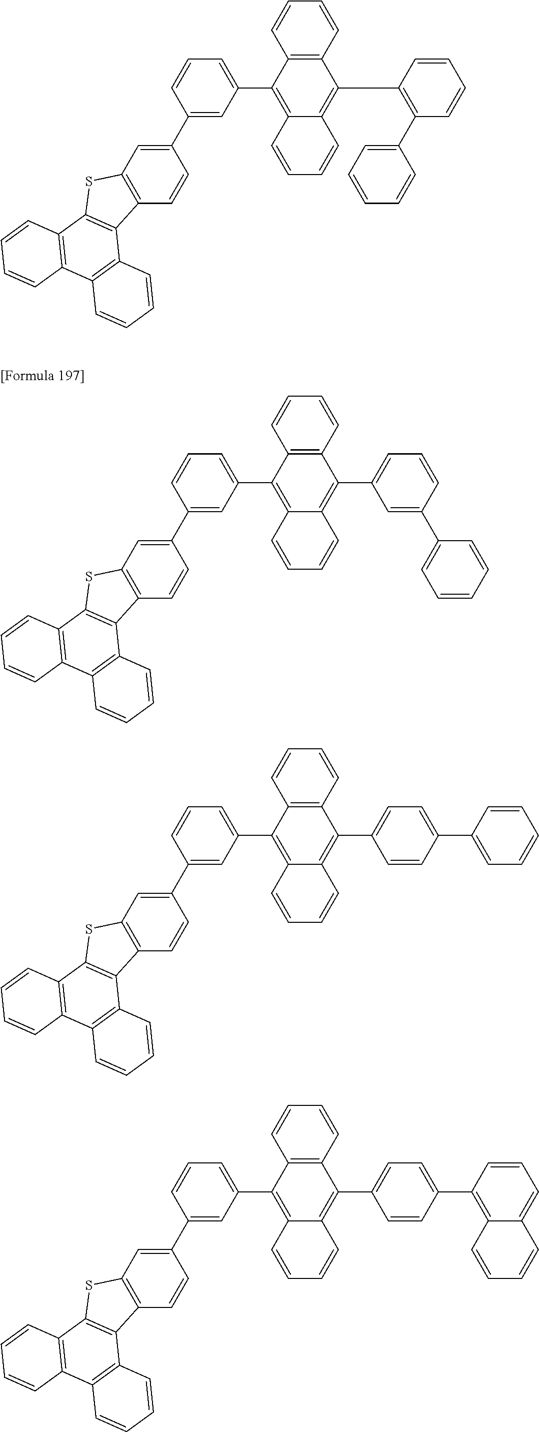 [SMILES: c1ccc(-c2ccccc2-c2c3ccccc3c(-c3cccc(-c4ccc5c(c4)sc4c6ccccc6c6ccccc6c54)c3)c3ccccc23)cc1 ; c1ccc(-c2cccc(-c3c4ccccc4c(-c4cccc(-c5ccc6c(c5)sc5c7ccccc7c7ccccc7c65)c4)c4ccccc34)c2)cc1 ; c1ccc(-c2ccc(-c3c4ccccc4c(-c4cccc(-c5ccc6c(c5)sc5c7ccccc7c7ccccc7c65)c4)c4ccccc34)cc2)cc1 ; c1cc(-c2ccc3c(c2)sc2c4ccccc4c4ccccc4c32)cc(-c2c3ccccc3c(-c3ccc(-c4cccc5ccccc45)cc3)c3ccccc23)c1]

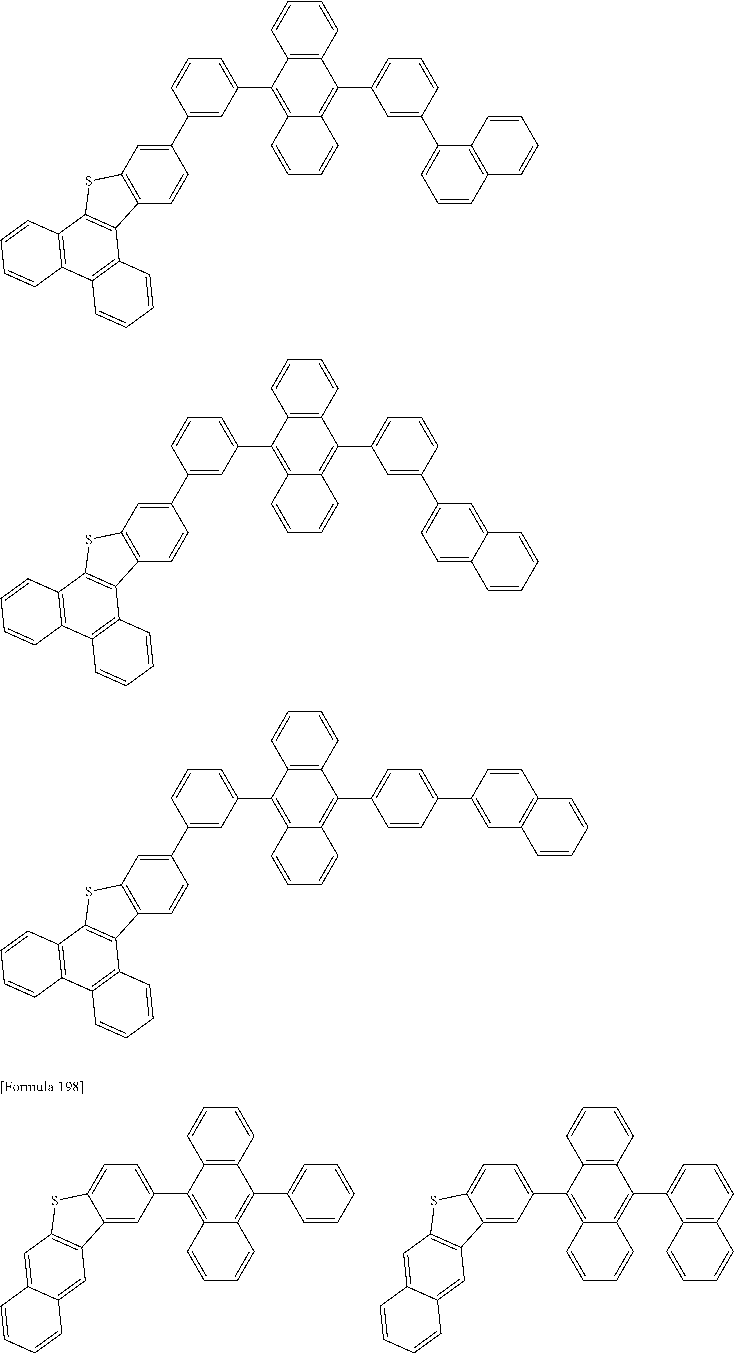 [SMILES: c1cc(-c2ccc3c(c2)sc2c4ccccc4c4ccccc4c32)cc(-c2c3ccccc3c(-c3cccc(-c4cccc5ccccc45)c3)c3ccccc23)c1 ; c1cc(-c2ccc3ccccc3c2)cc(-c2c3ccccc3c(-c3cccc(-c4ccc5c(c4)sc4c6ccccc6c6ccccc6c54)c3)c3ccccc23)c1 ; c1cc(-c2ccc3c(c2)sc2c4ccccc4c4ccccc4c32)cc(-c2c3ccccc3c(-c3ccc(-c4ccc5ccccc5c4)cc3)c3ccccc23)c1 ; c1ccc(-c2c3ccccc3c(-c3ccc4sc5cc6ccccc6cc5c4c3)c3ccccc23)cc1 ; c1ccc2cc3c(cc2c1)sc1ccc(-c2c4ccccc4c(-c4cccc5ccccc45)c4ccccc24)cc13]

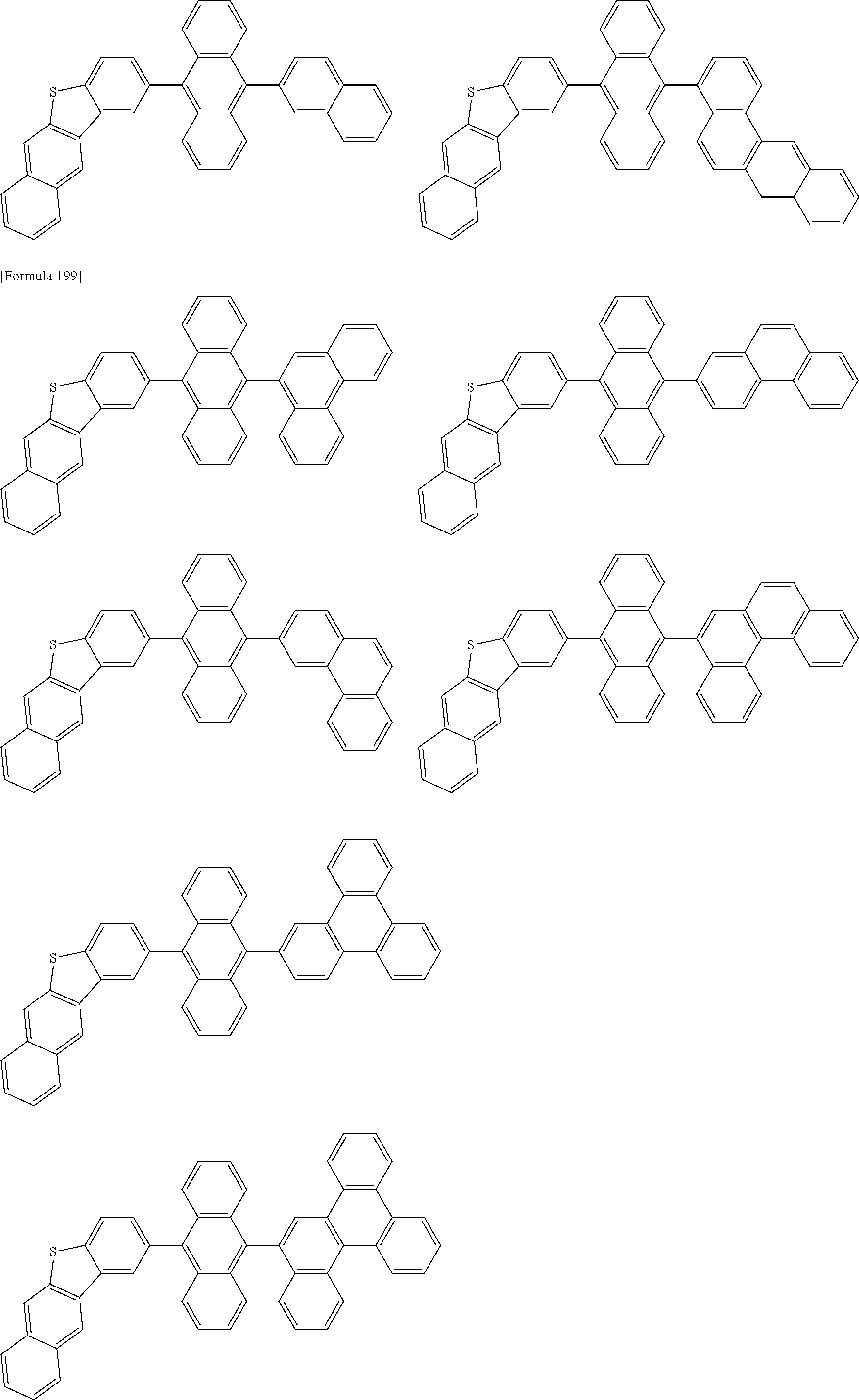

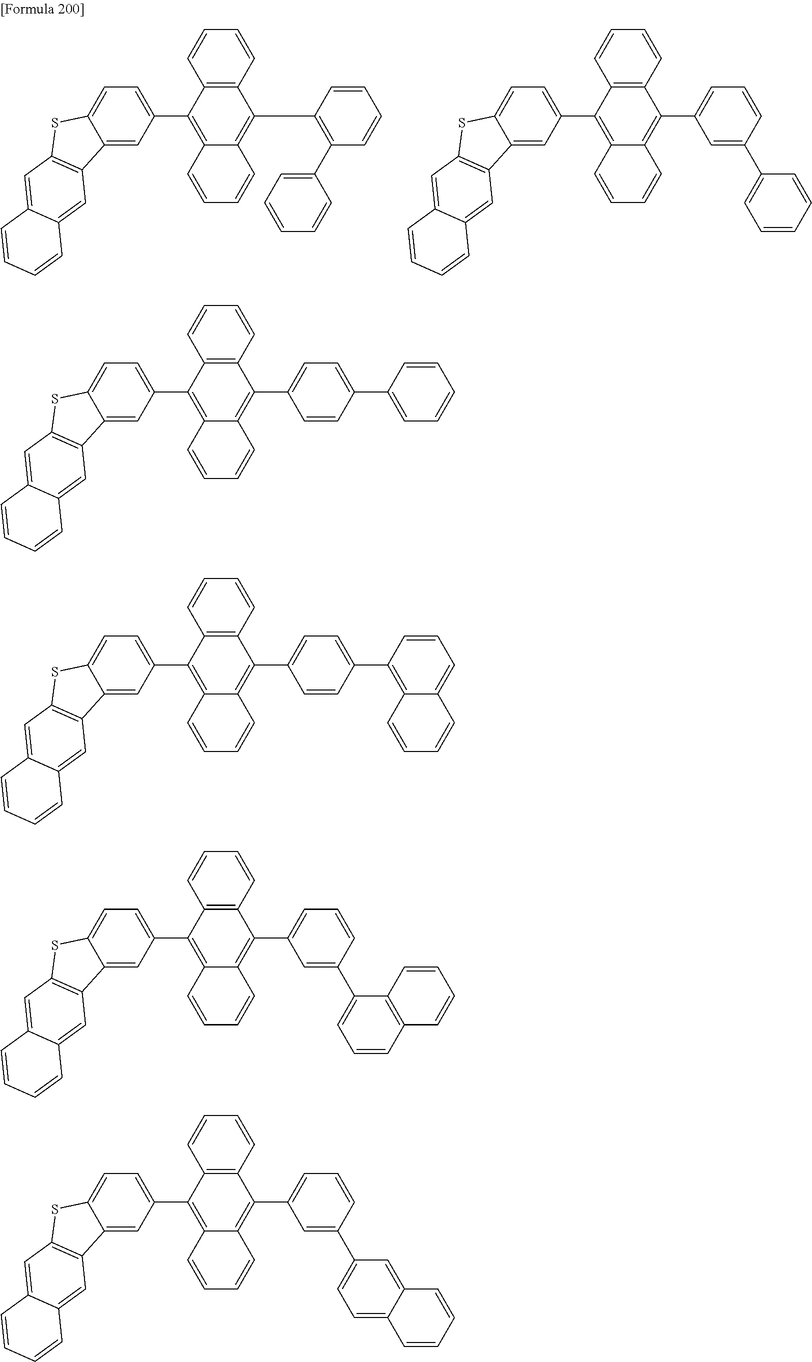 [SMILES: c1ccc(-c2ccccc2-c2c3ccccc3c(-c3ccc4sc5cc6ccccc6cc5c4c3)c3ccccc23)cc1 ; c1ccc(-c2cccc(-c3c4ccccc4c(-c4ccc5sc6cc7ccccc7cc6c5c4)c4ccccc34)c2)cc1 ; c1ccc(-c2ccc(-c3c4ccccc4c(-c4ccc5sc6cc7ccccc7cc6c5c4)c4ccccc34)cc2)cc1 ; c1ccc2cc3c(cc2c1)sc1ccc(-c2c4ccccc4c(-c4ccc(-c5cccc6ccccc56)cc4)c4ccccc24)cc13 ; c1cc(-c2cccc3ccccc23)cc(-c2c3ccccc3c(-c3ccc4sc5cc6ccccc6cc5c4c3)c3ccccc23)c1 ; c1cc(-c2ccc3ccccc3c2)cc(-c2c3ccccc3c(-c3ccc4sc5cc6ccccc6cc5c4c3)c3ccccc23)c1]

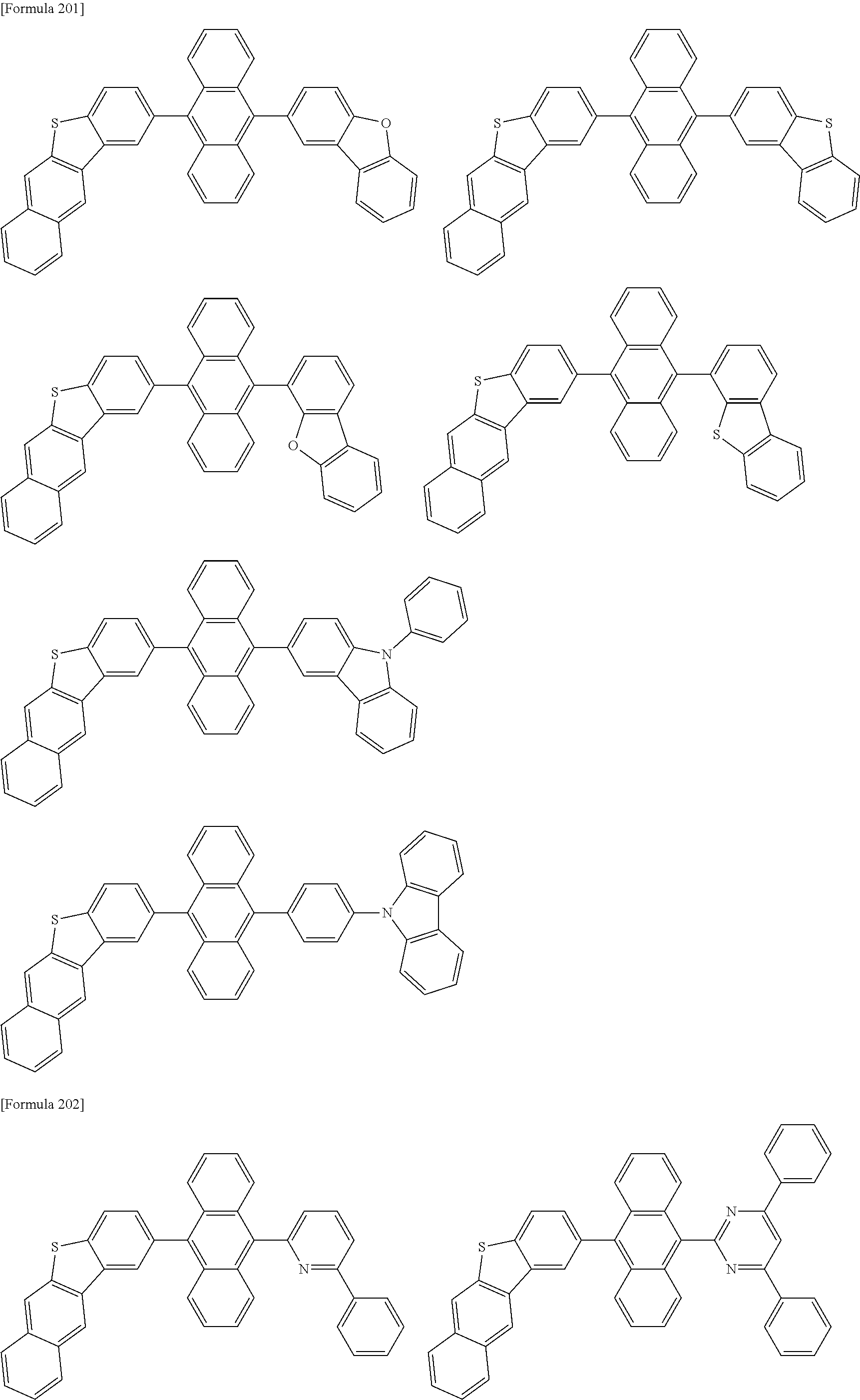

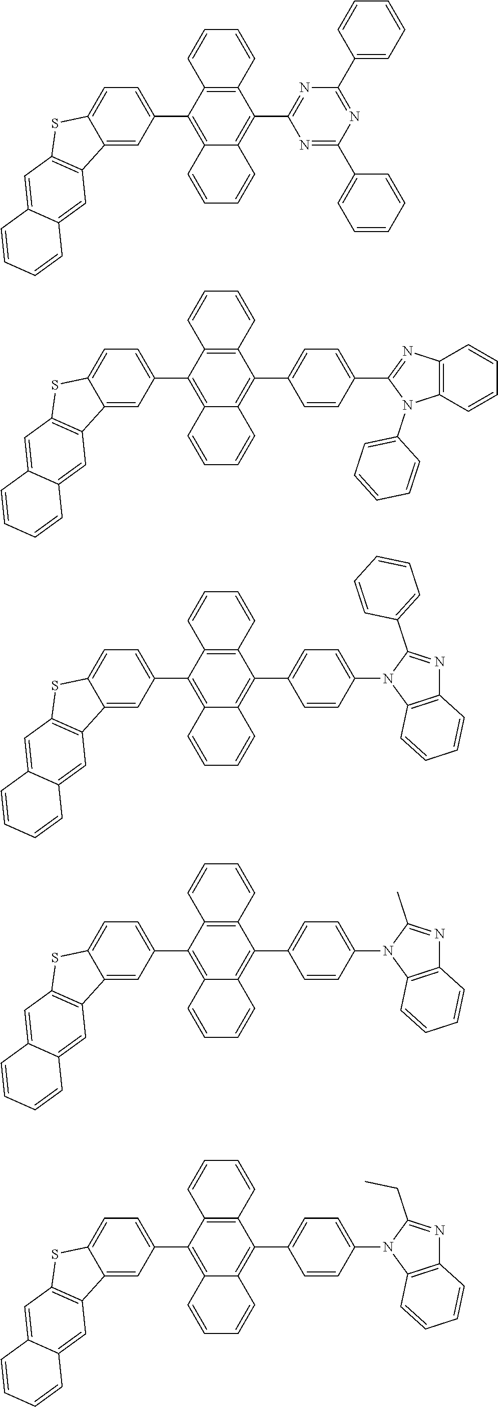 [SMILES: c1ccc(-c2nc(-c3ccccc3)nc(-c3c4ccccc4c(-c4ccc5sc6cc7ccccc7cc6c5c4)c4ccccc34)n2)cc1 ; c1ccc(-n2c(-c3ccc(-c4c5ccccc5c(-c5ccc6sc7cc8ccccc8cc7c6c5)c5ccccc45)cc3)nc3ccccc32)cc1 ; c1ccc(-c2nc3ccccc3n2-c2ccc(-c3c4ccccc4c(-c4ccc5sc6cc7ccccc7cc6c5c4)c4ccccc34)cc2)cc1 ; Cc1nc2ccccc2n1-c1ccc(-c2c3ccccc3c(-c3ccc4sc5cc6ccccc6cc5c4c3)c3ccccc23)cc1 ; CCc1nc2ccccc2n1-c1ccc(-c2c3ccccc3c(-c3ccc4sc5cc6ccccc6cc5c4c3)c3ccccc23)cc1]

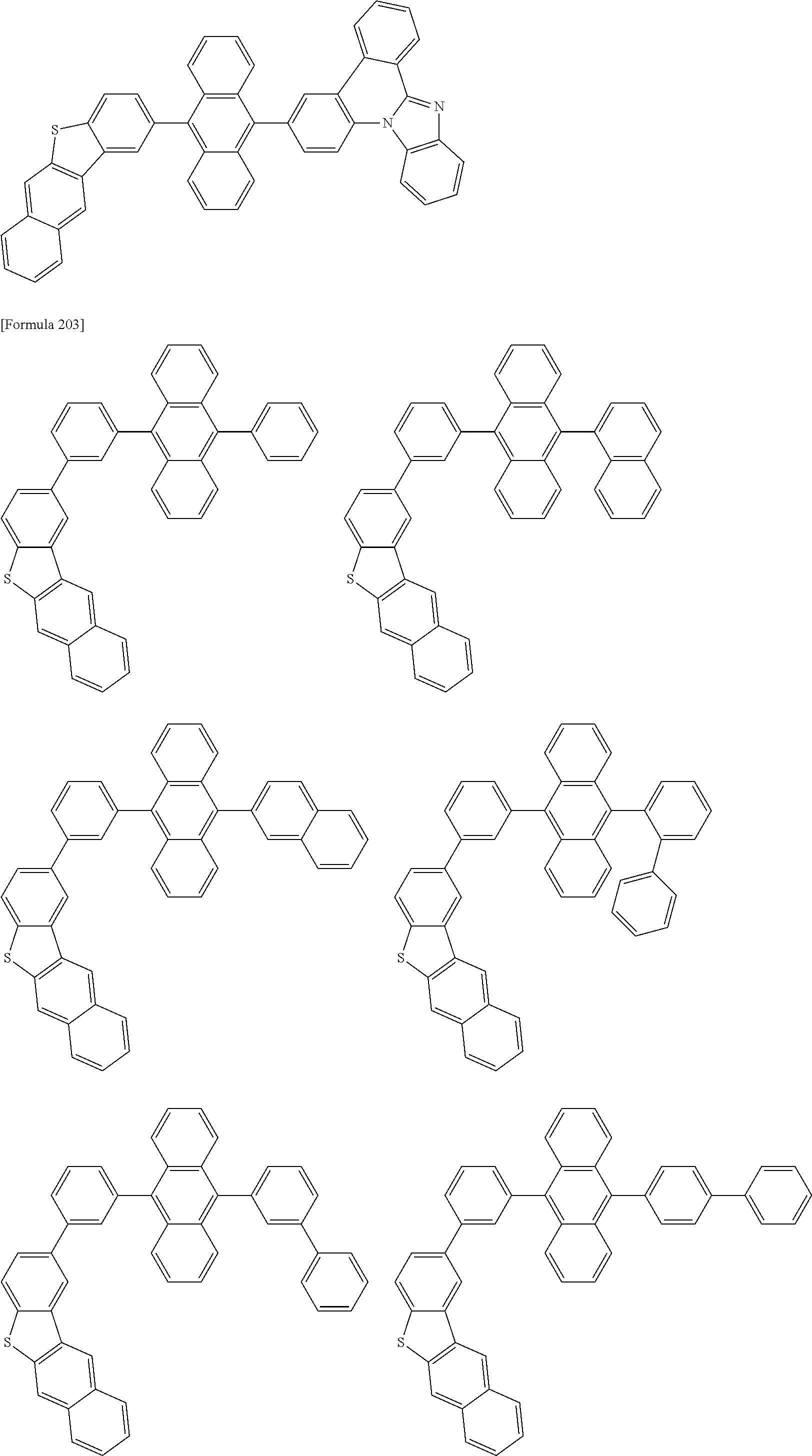

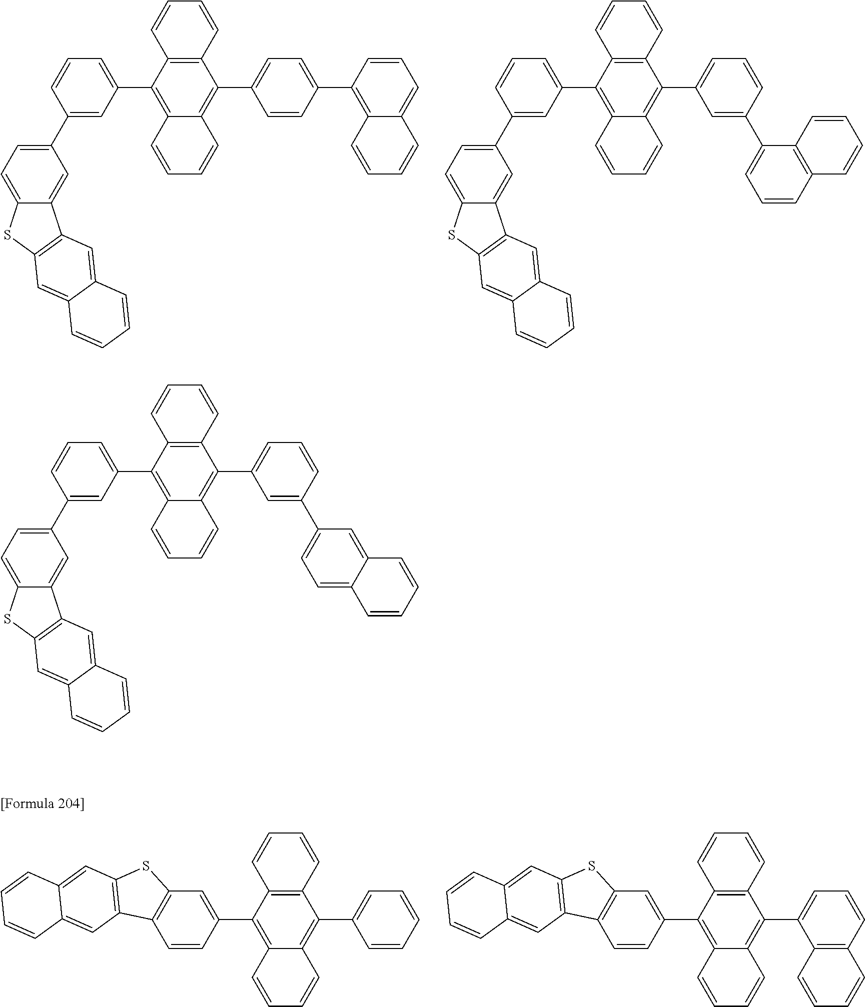

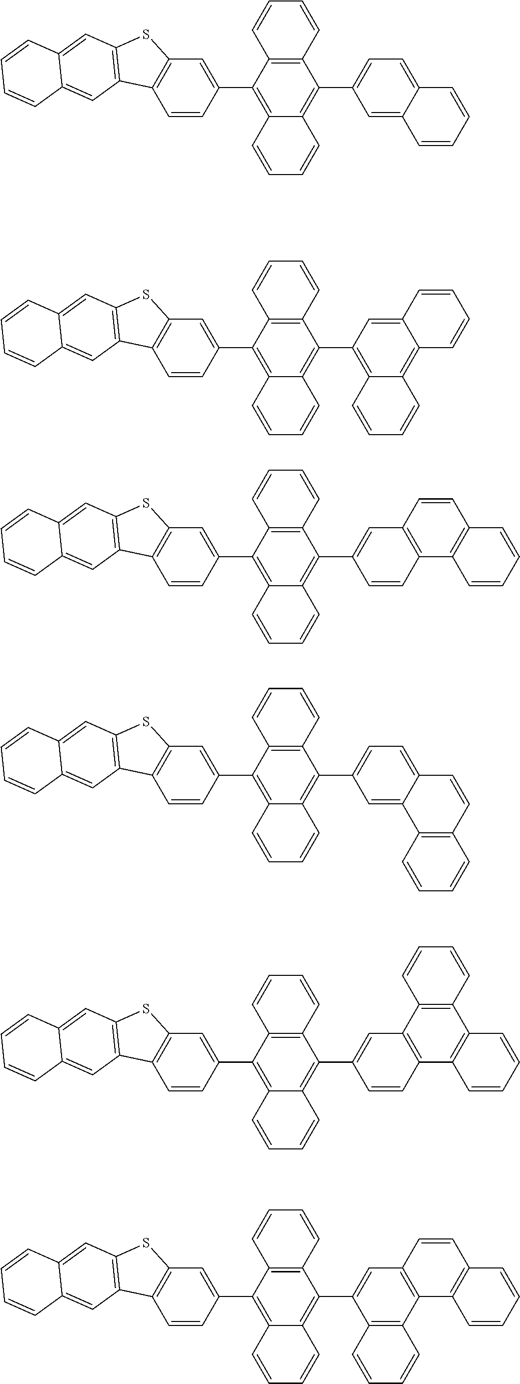

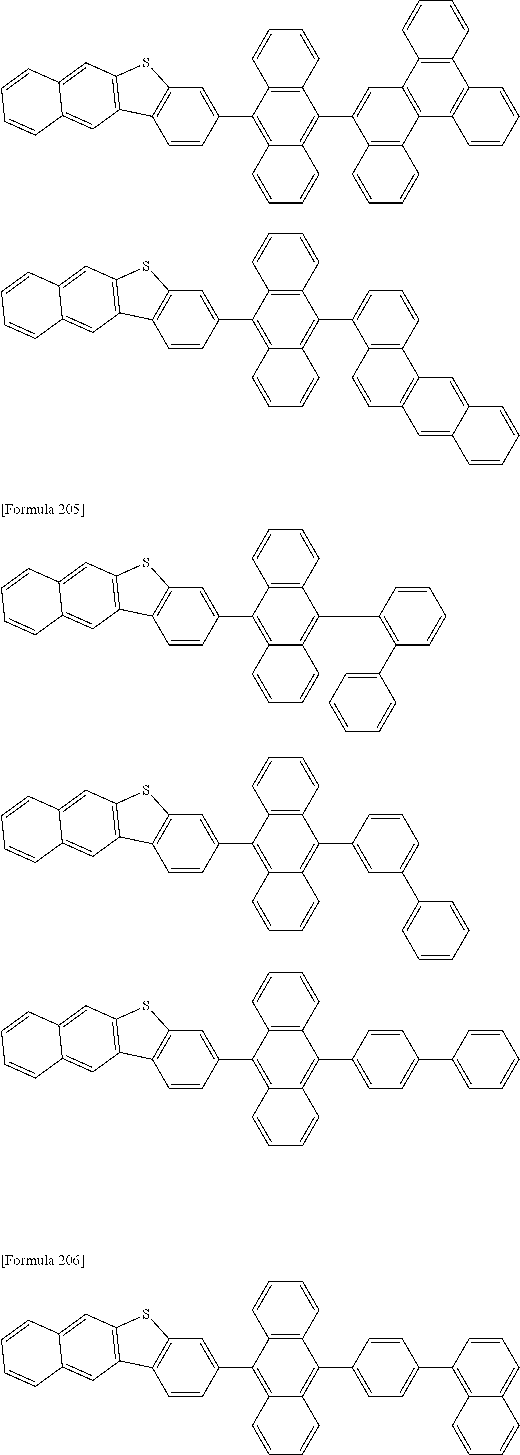

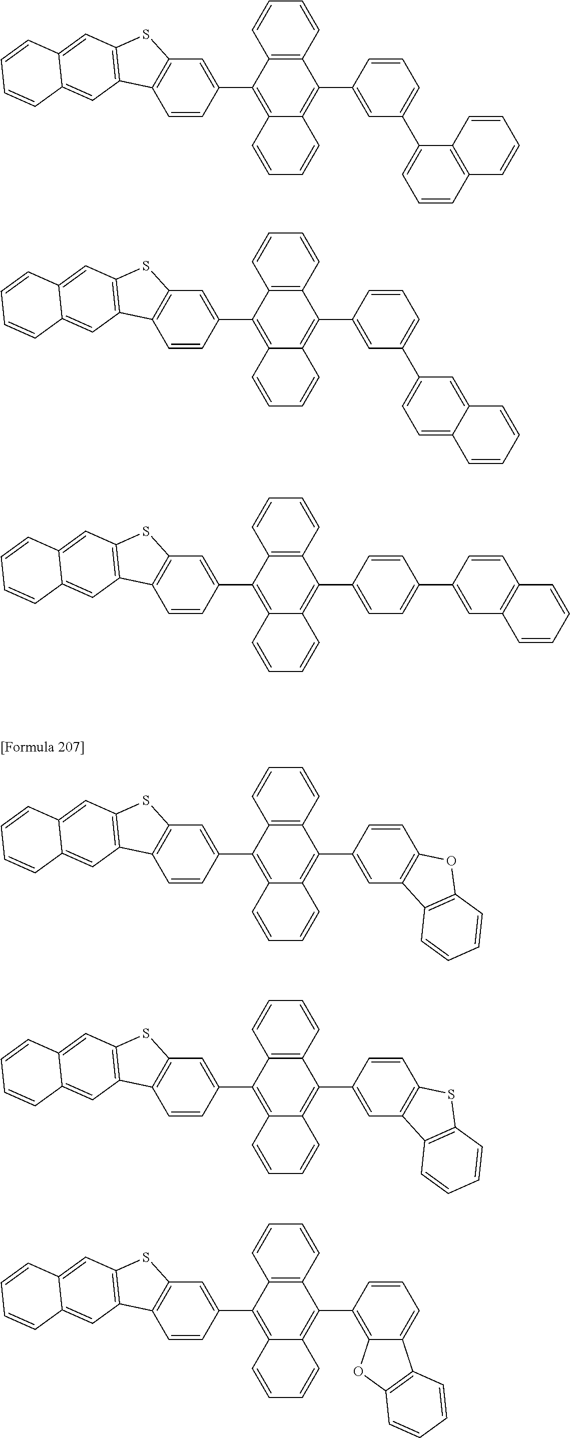 [SMILES: c1cc(-c2cccc3ccccc23)cc(-c2c3ccccc3c(-c3ccc4c(c3)sc3cc5ccccc5cc34)c3ccccc23)c1 ; c1cc(-c2ccc3ccccc3c2)cc(-c2c3ccccc3c(-c3ccc4c(c3)sc3cc5ccccc5cc34)c3ccccc23)c1 ; c1ccc2cc(-c3ccc(-c4c5ccccc5c(-c5ccc6c(c5)sc5cc7ccccc7cc56)c5ccccc45)cc3)ccc2c1 ; c1ccc2cc3c(cc2c1)sc1cc(-c2c4ccccc4c(-c4ccc5oc6ccccc6c5c4)c4ccccc24)ccc13 ; c1ccc2cc3c(cc2c1)sc1cc(-c2c4ccccc4c(-c4ccc5sc6ccccc6c5c4)c4ccccc24)ccc13 ; c1ccc2cc3c(cc2c1)sc1cc(-c2c4ccccc4c(-c4cccc5c4oc4ccccc45)c4ccccc24)ccc13]

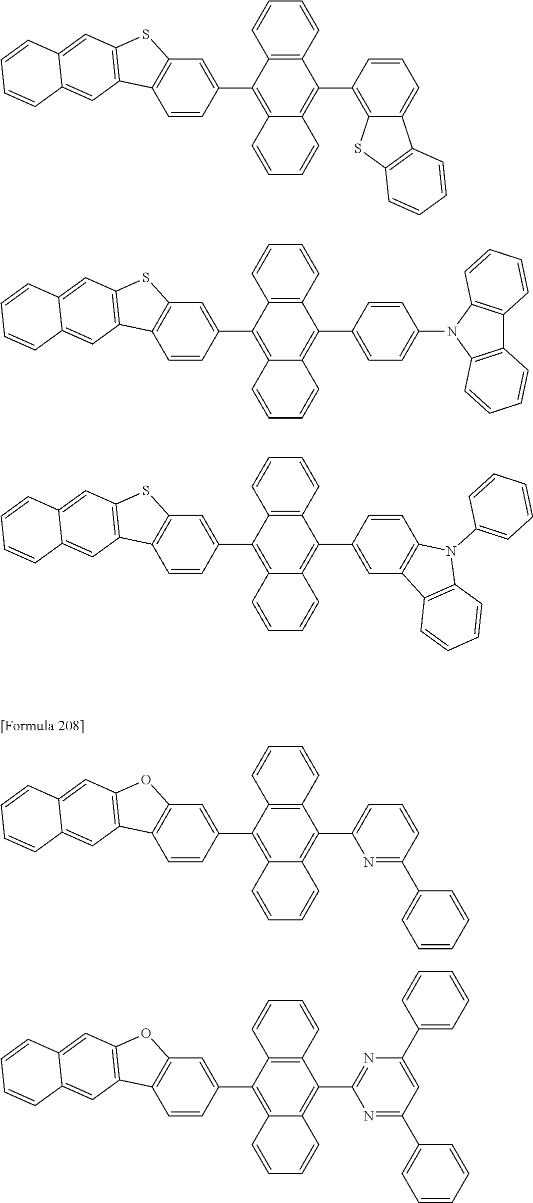

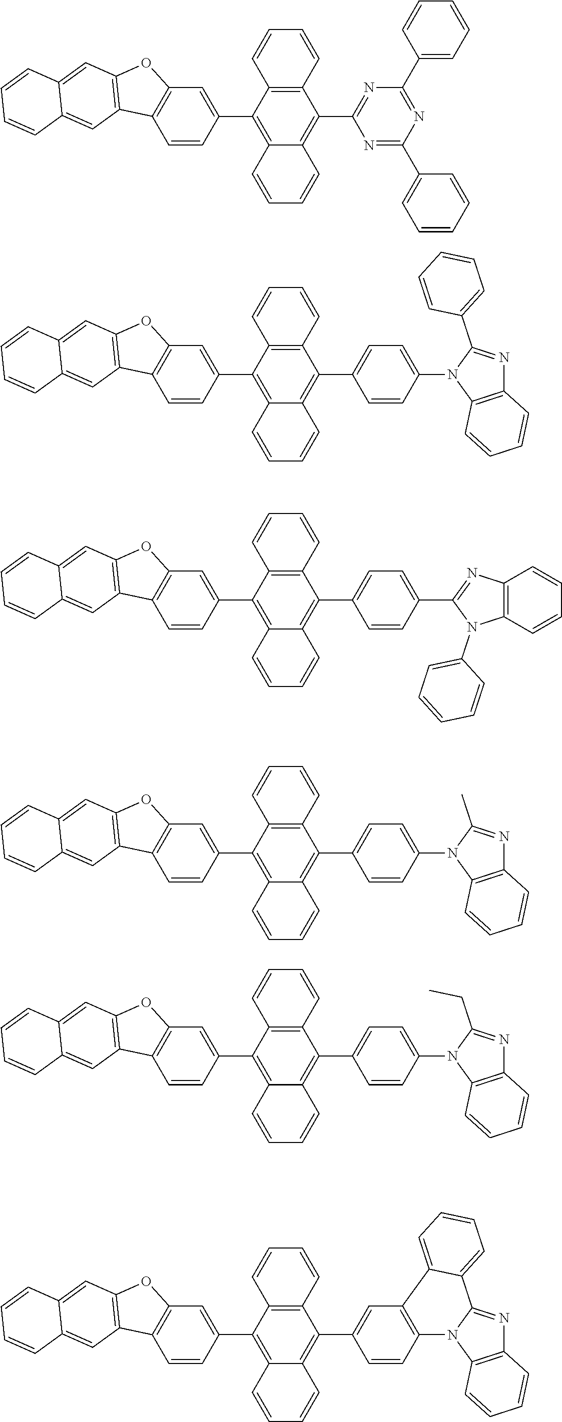

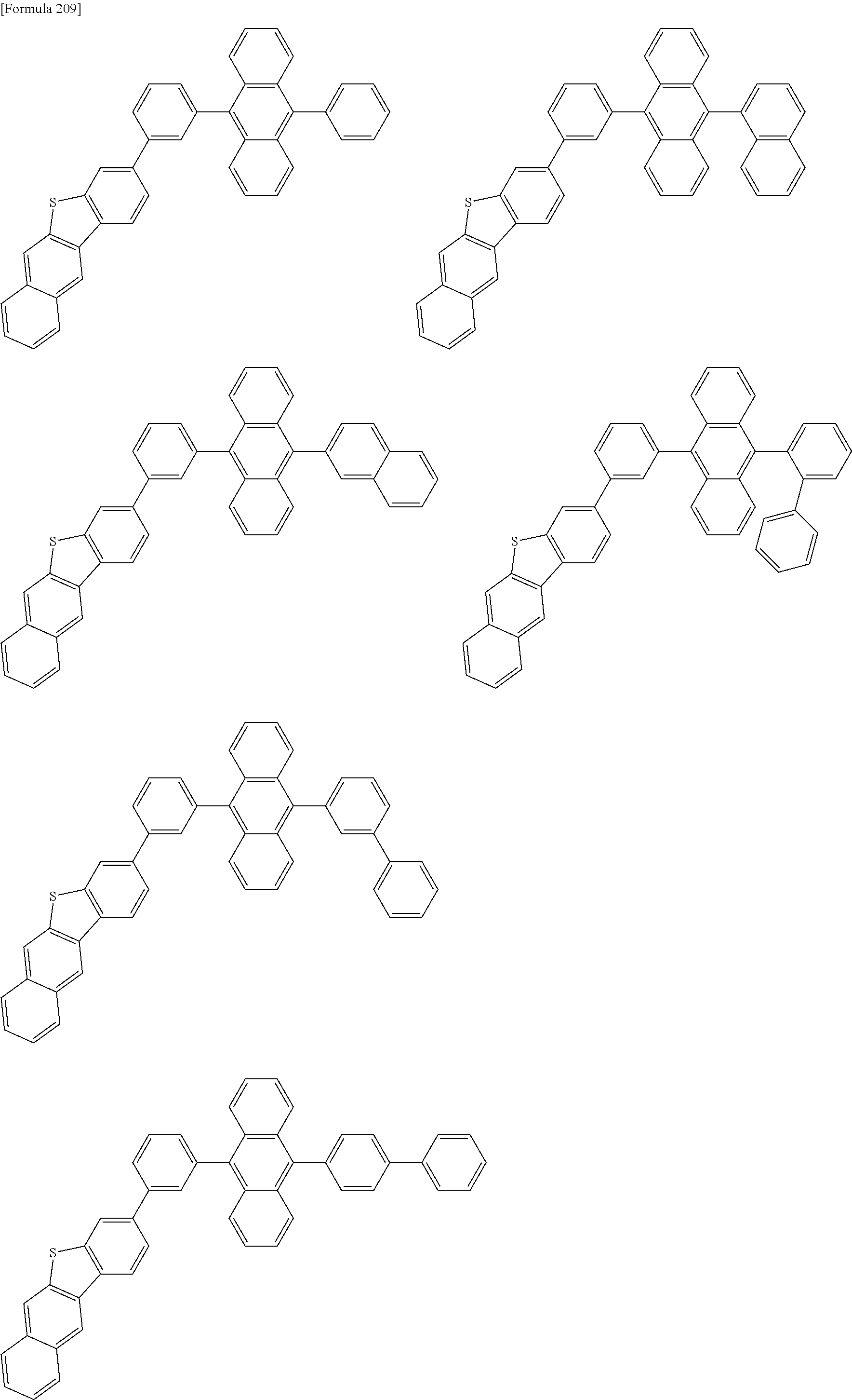 [SMILES: c1ccc(-c2c3ccccc3c(-c3cccc(-c4ccc5c(c4)sc4cc6ccccc6cc45)c3)c3ccccc23)cc1 ; c1cc(-c2ccc3c(c2)sc2cc4ccccc4cc23)cc(-c2c3ccccc3c(-c3cccc4ccccc34)c3ccccc23)c1 ; c1cc(-c2ccc3c(c2)sc2cc4ccccc4cc23)cc(-c2c3ccccc3c(-c3ccc4ccccc4c3)c3ccccc23)c1 ; c1ccc(-c2ccccc2-c2c3ccccc3c(-c3cccc(-c4ccc5c(c4)sc4cc6ccccc6cc45)c3)c3ccccc23)cc1 ; c1ccc(-c2cccc(-c3c4ccccc4c(-c4cccc(-c5ccc6c(c5)sc5cc7ccccc7cc56)c4)c4ccccc34)c2)cc1 ; c1ccc(-c2ccc(-c3c4ccccc4c(-c4cccc(-c5ccc6c(c5)sc5cc7ccccc7cc56)c4)c4ccccc34)cc2)cc1]

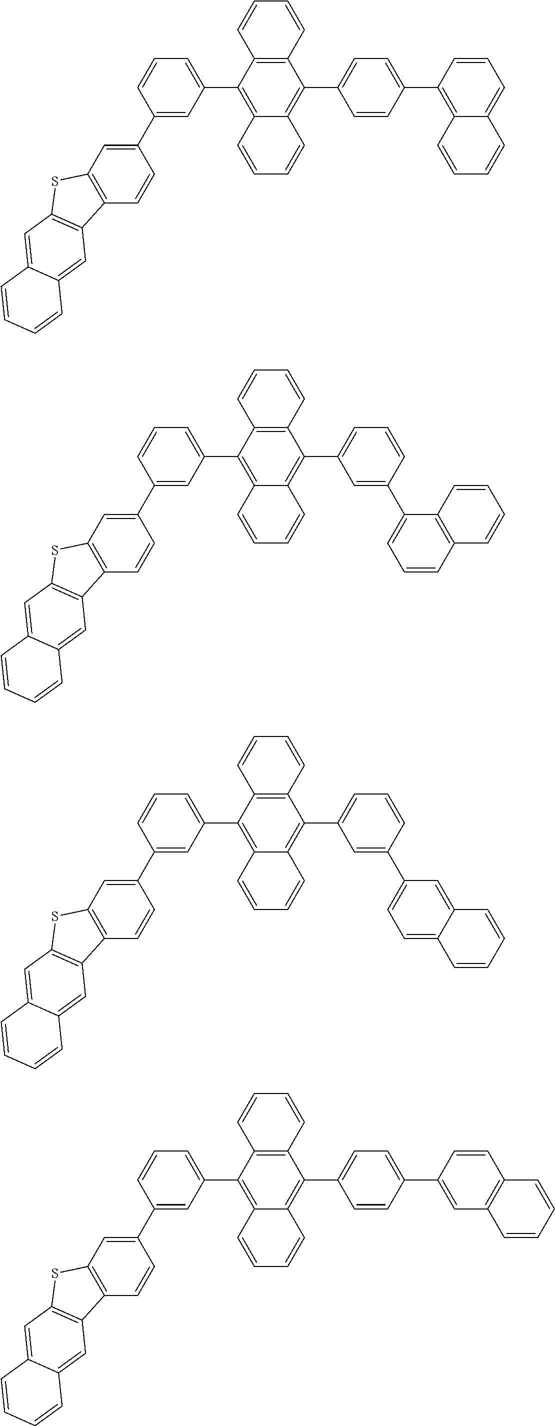 [SMILES: c1cc(-c2ccc3c(c2)sc2cc4ccccc4cc23)cc(-c2c3ccccc3c(-c3ccc(-c4cccc5ccccc45)cc3)c3ccccc23)c1 ; c1cc(-c2ccc3c(c2)sc2cc4ccccc4cc23)cc(-c2c3ccccc3c(-c3cccc(-c4cccc5ccccc45)c3)c3ccccc23)c1 ; c1cc(-c2ccc3ccccc3c2)cc(-c2c3ccccc3c(-c3cccc(-c4ccc5c(c4)sc4cc6ccccc6cc45)c3)c3ccccc23)c1 ; c1cc(-c2ccc3c(c2)sc2cc4ccccc4cc23)cc(-c2c3ccccc3c(-c3ccc(-c4ccc5ccccc5c4)cc3)c3ccccc23)c1]

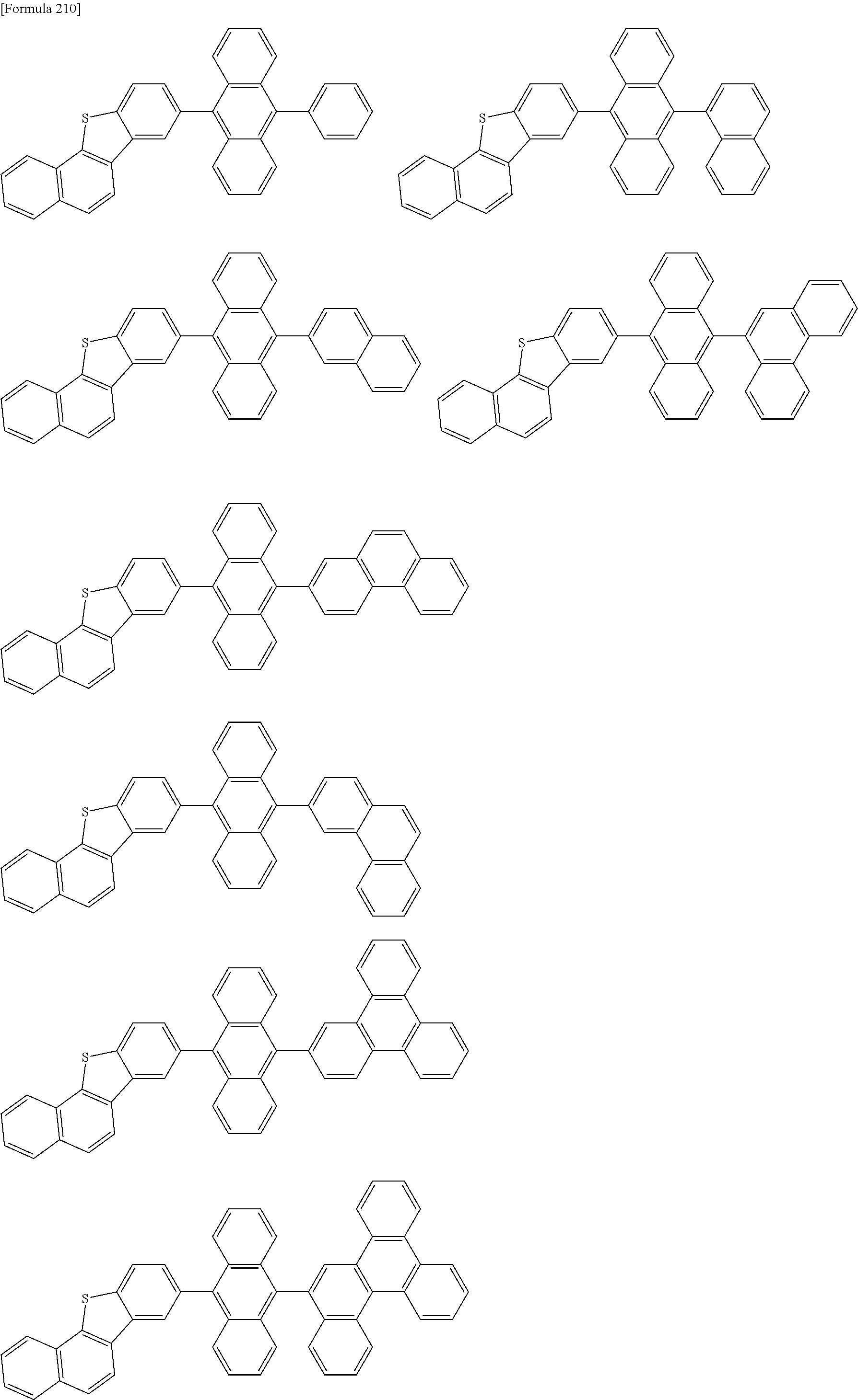

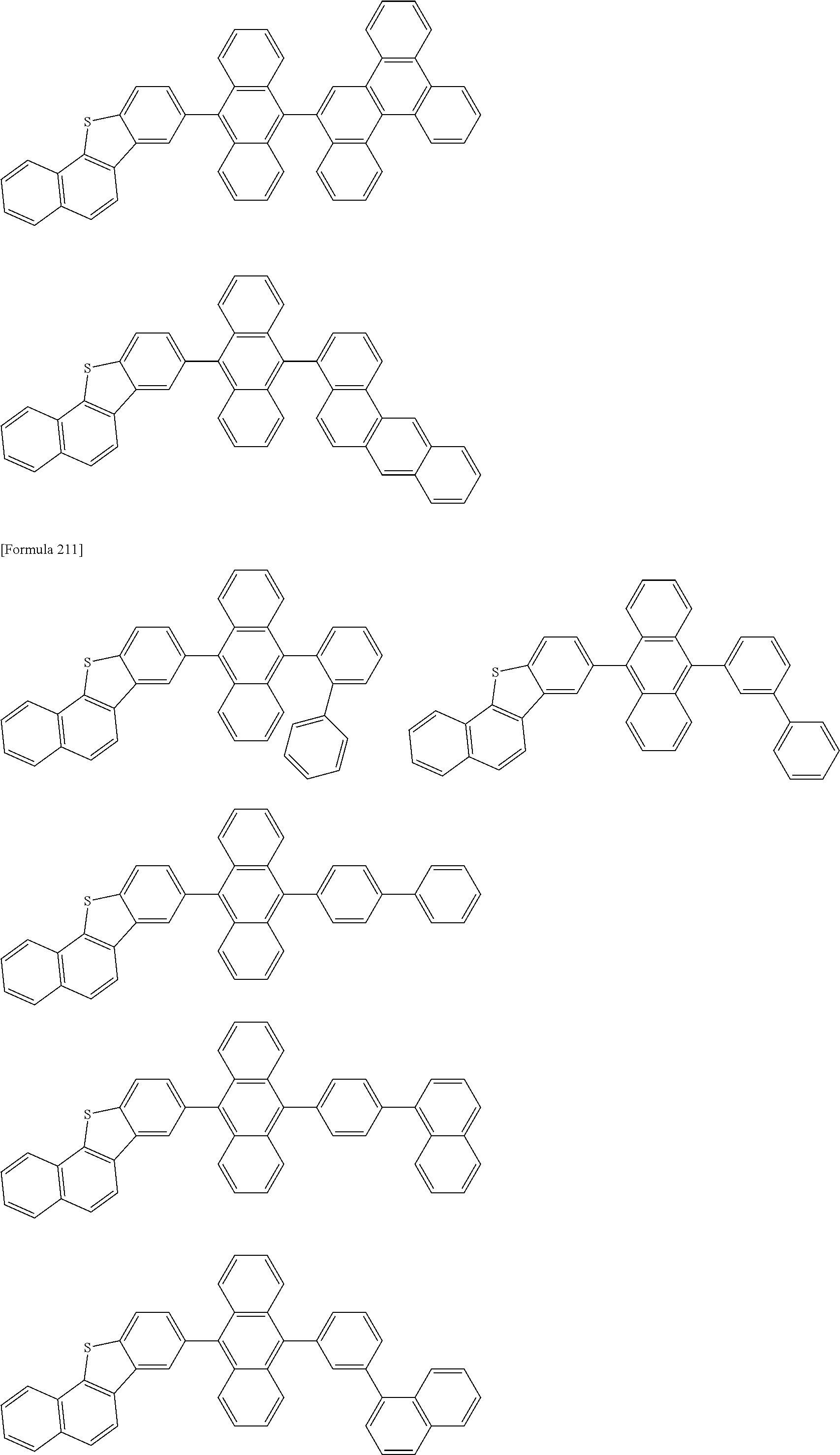

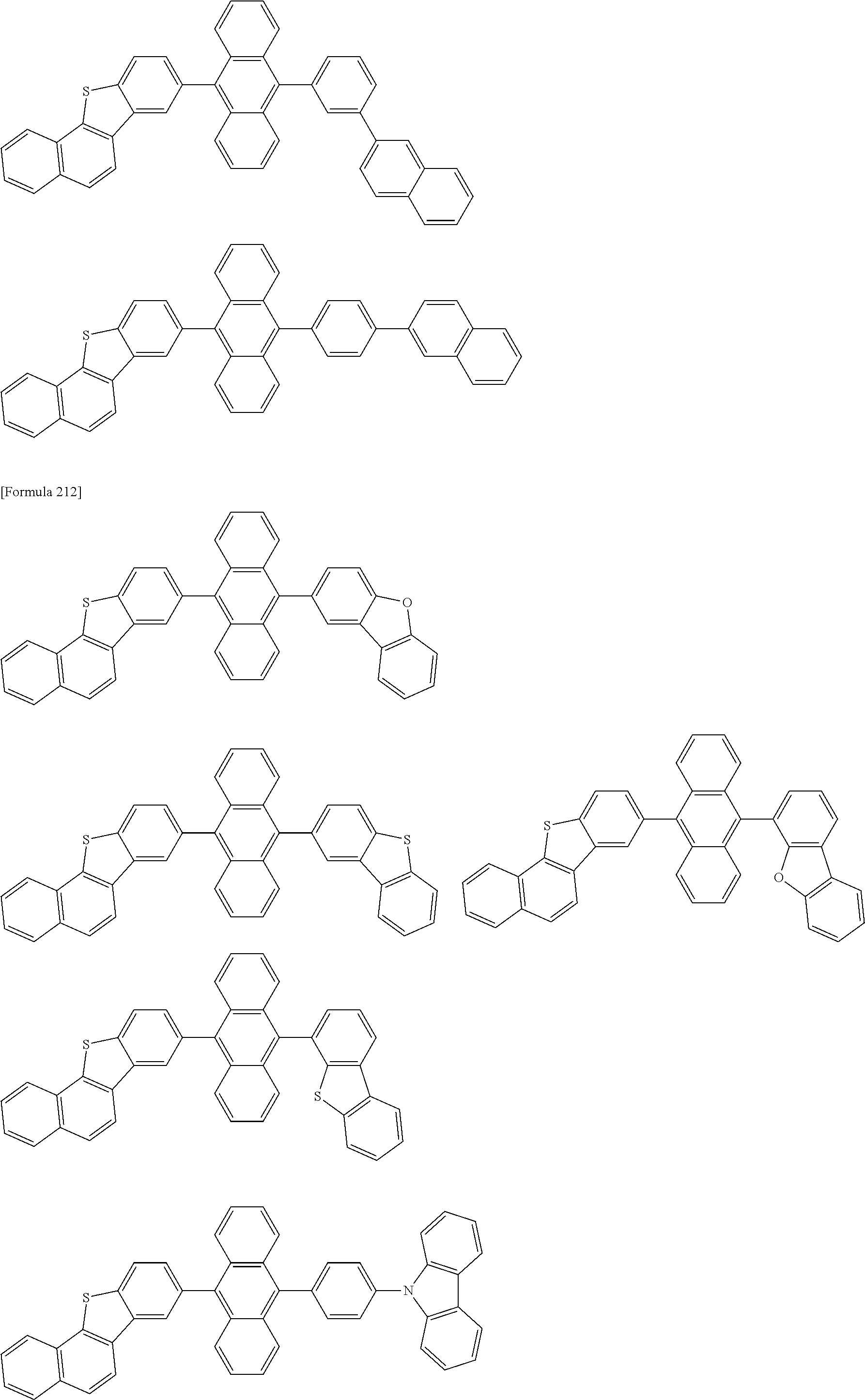

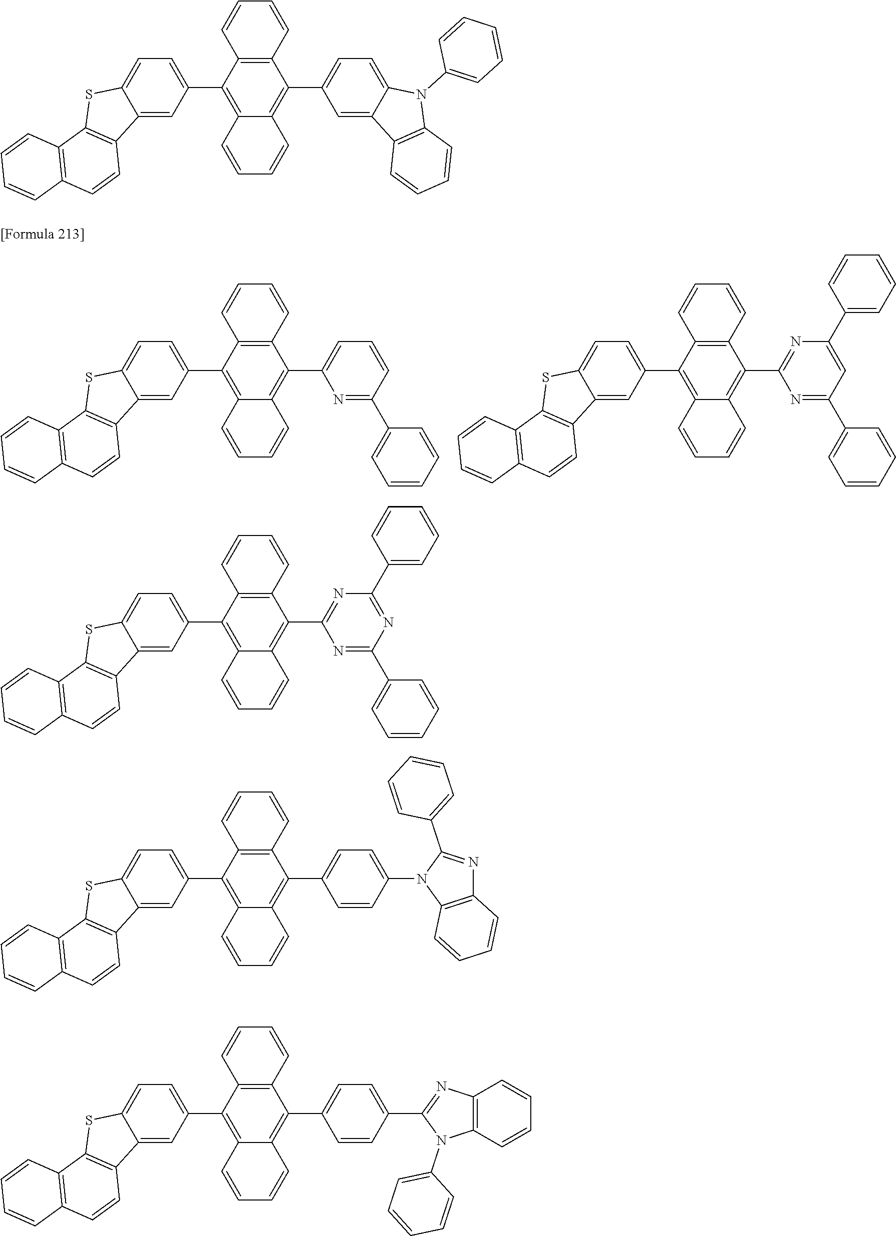

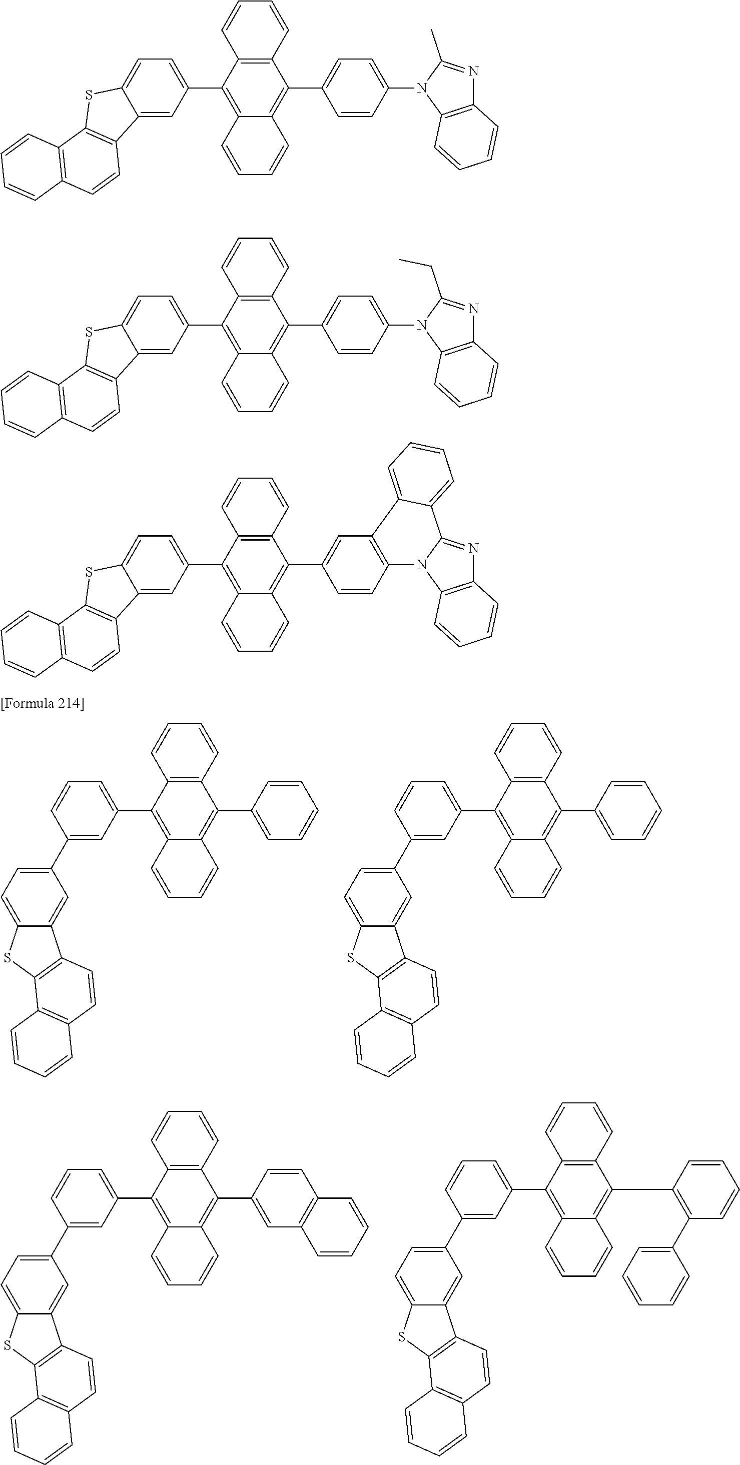

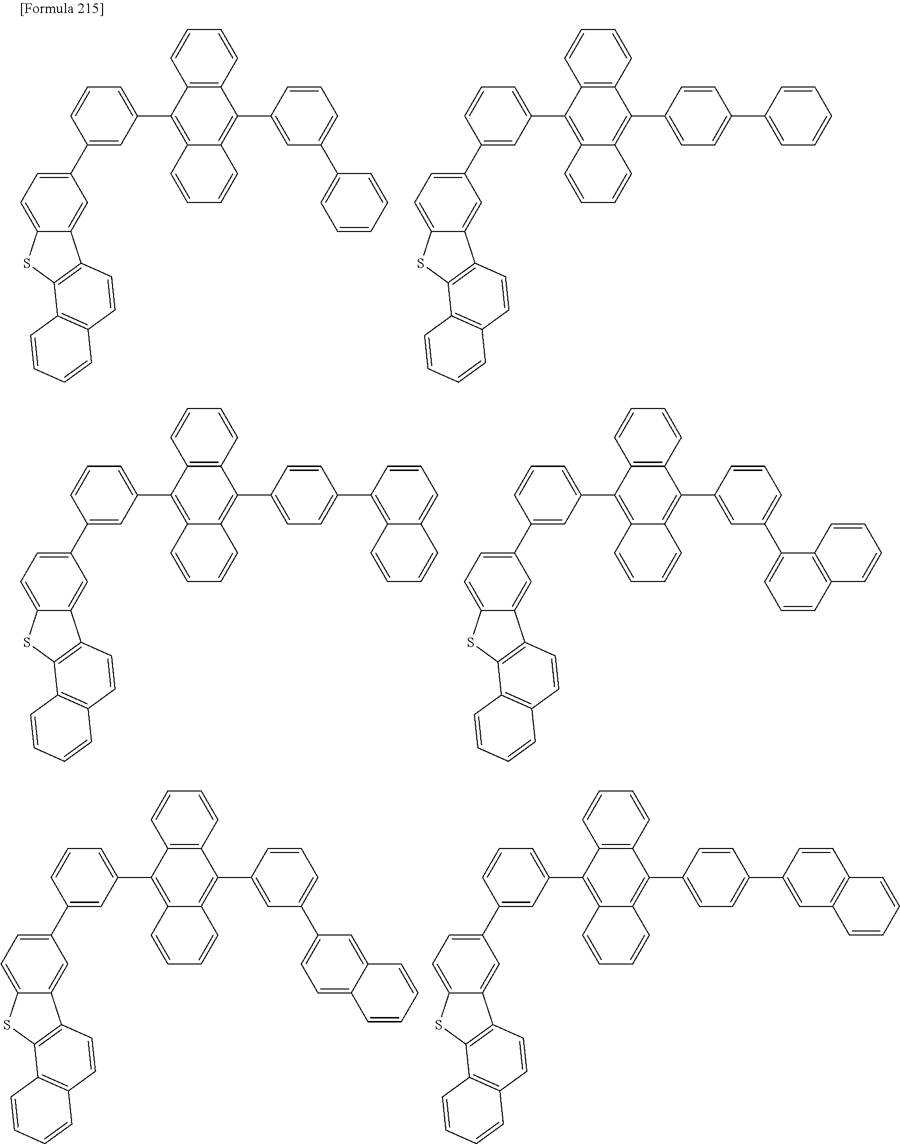

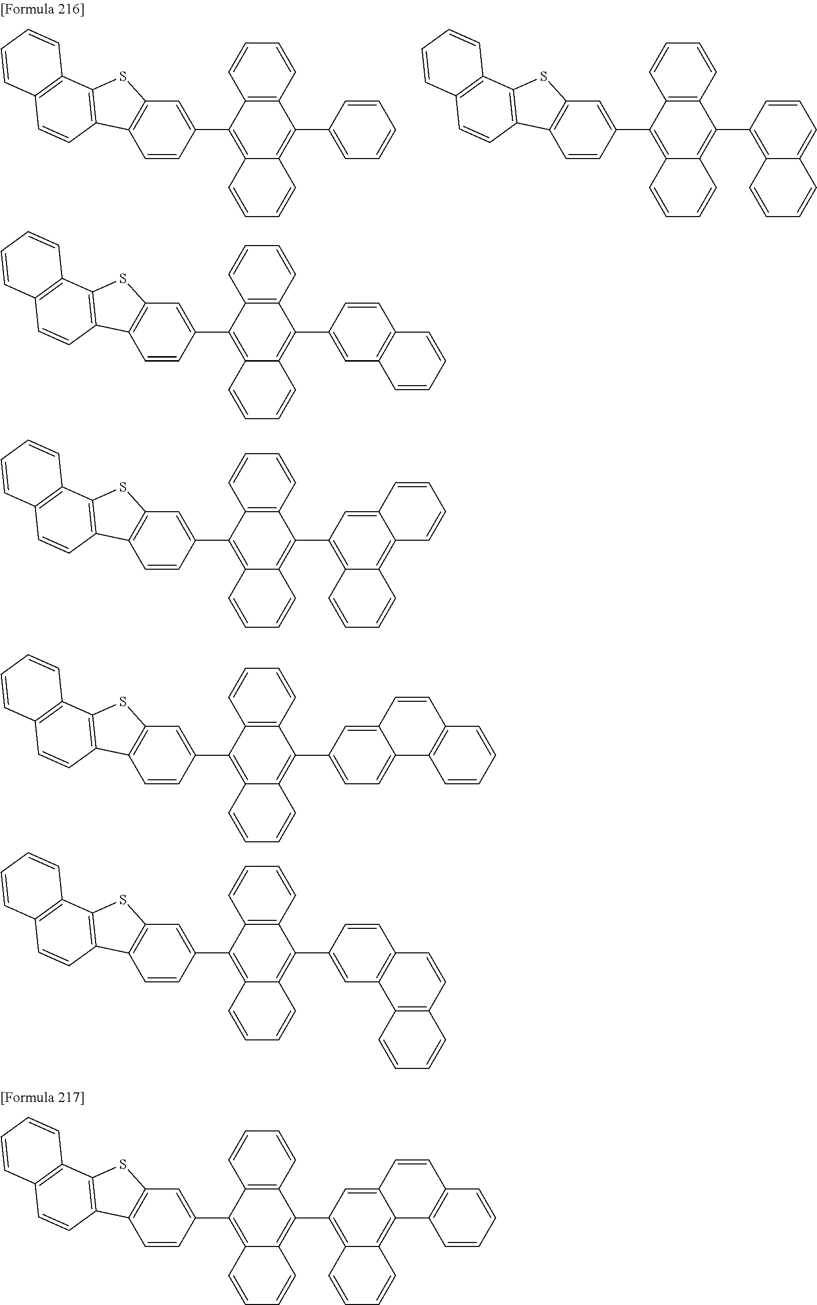

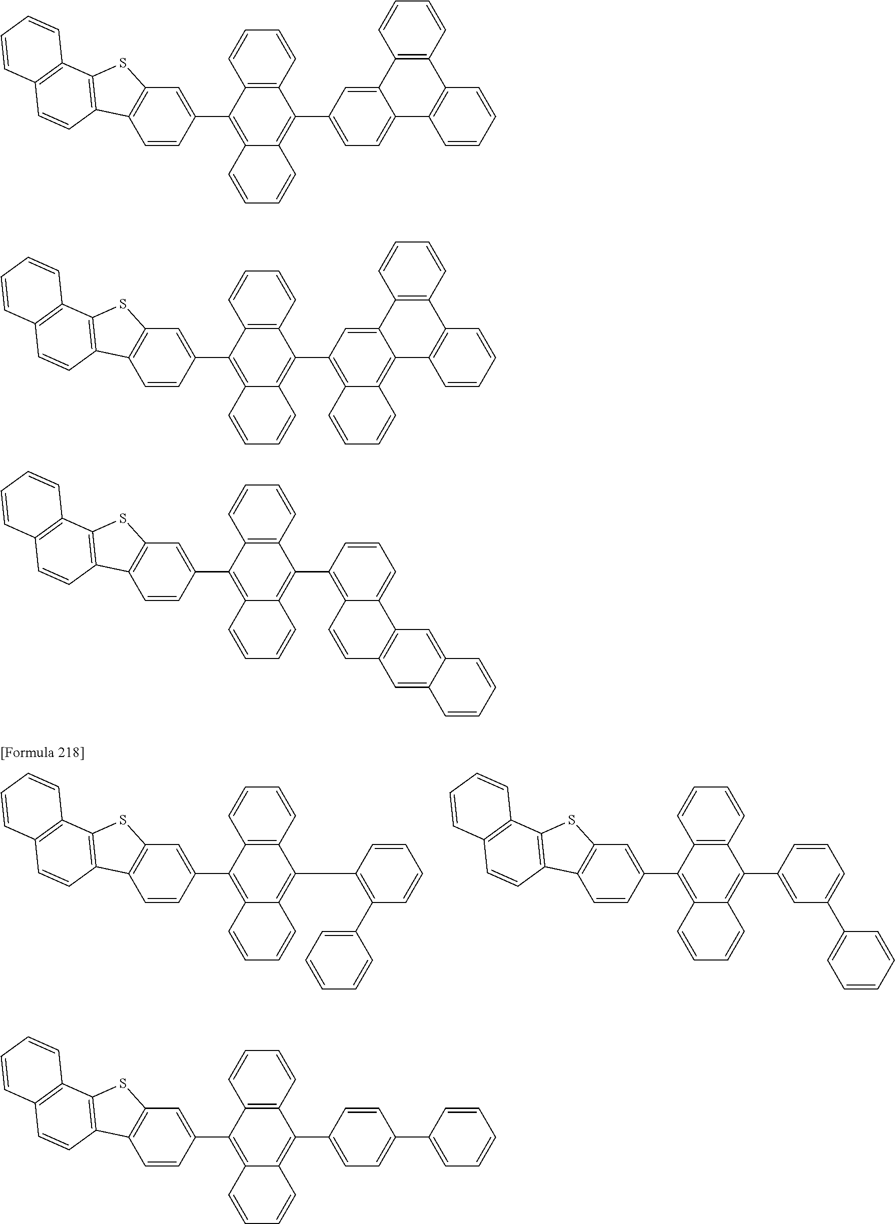

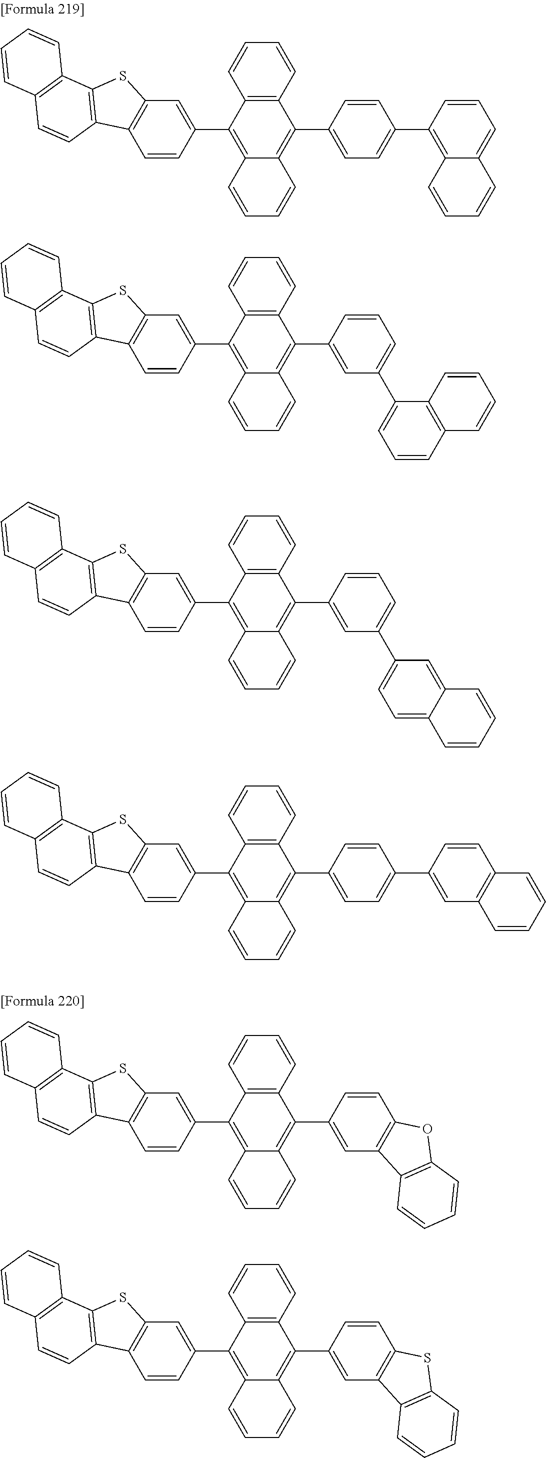

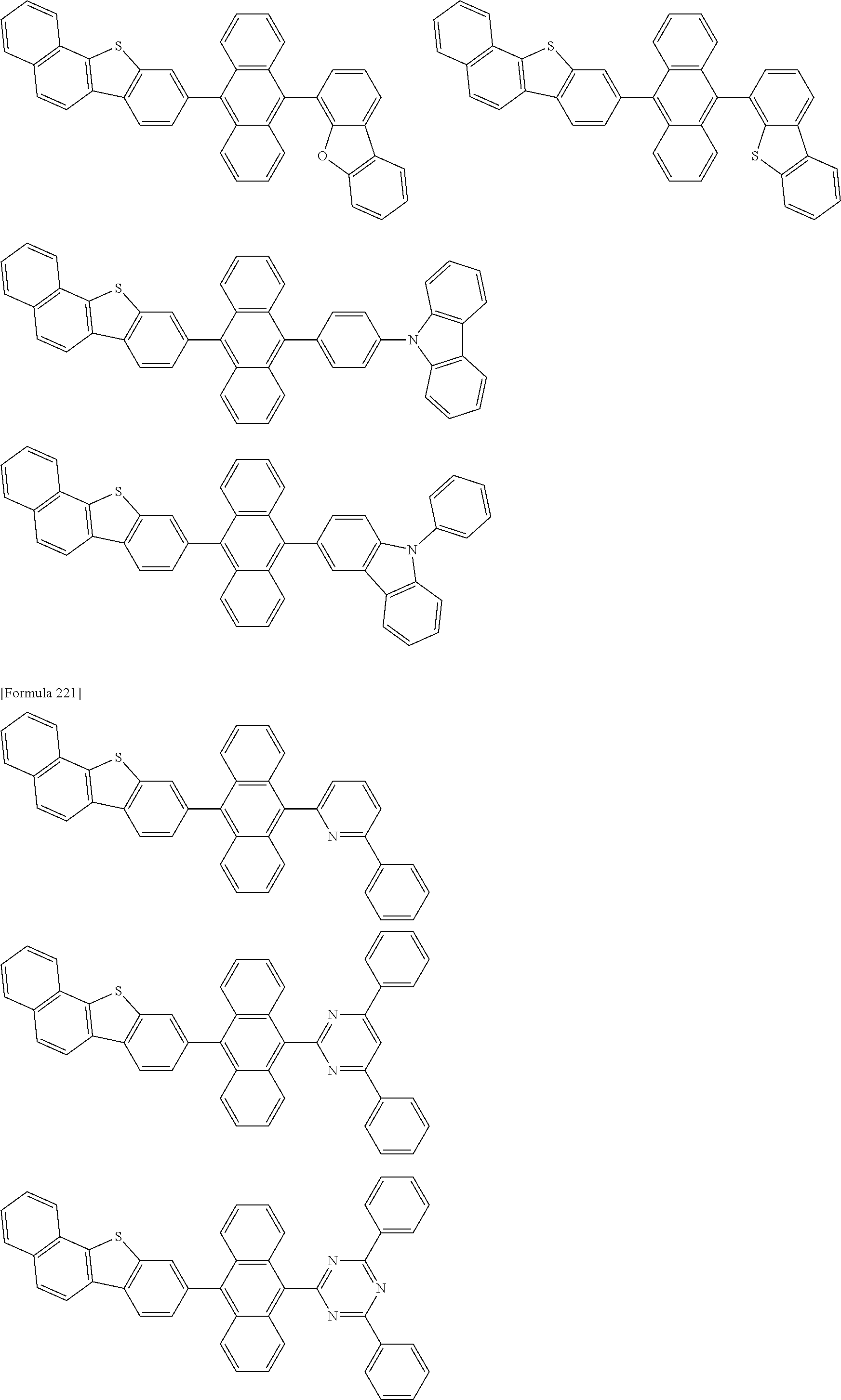

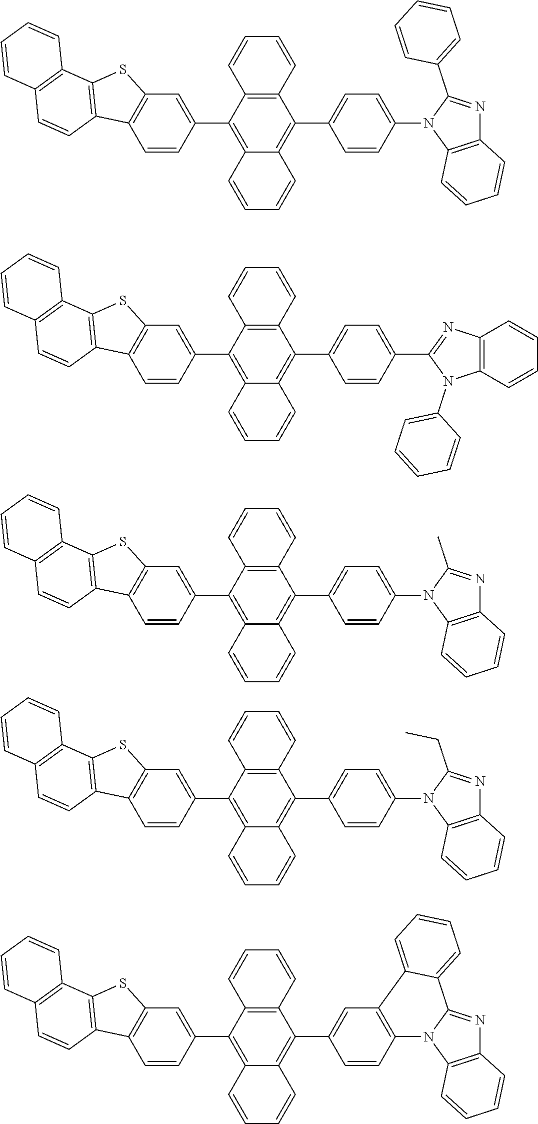

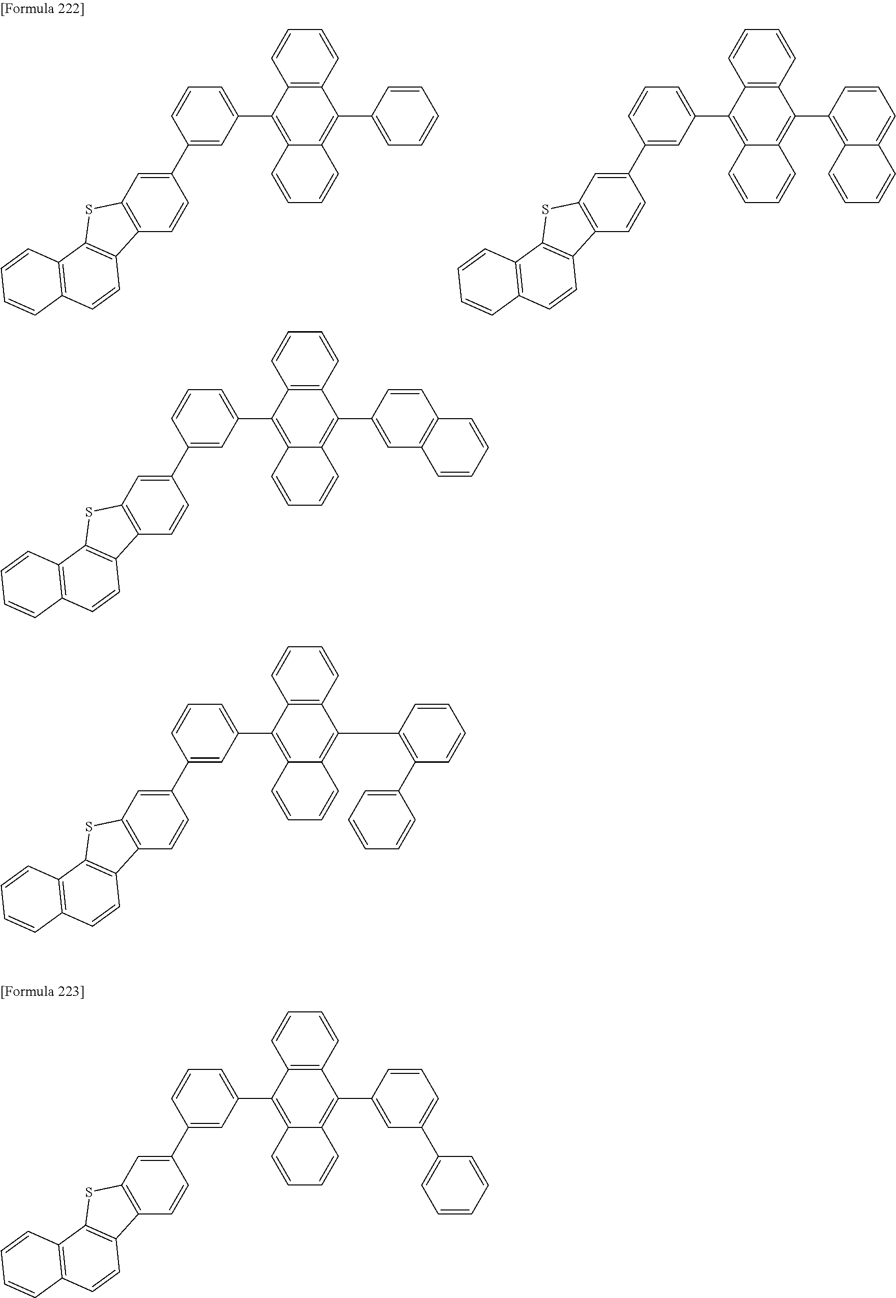 [SMILES: c1ccc(-c2c3ccccc3c(-c3cccc(-c4ccc5c(c4)sc4c6ccccc6ccc54)c3)c3ccccc23)cc1 ; c1cc(-c2ccc3c(c2)sc2c4ccccc4ccc32)cc(-c2c3ccccc3c(-c3cccc4ccccc34)c3ccccc23)c1 ; c1cc(-c2ccc3c(c2)sc2c4ccccc4ccc32)cc(-c2c3ccccc3c(-c3ccc4ccccc4c3)c3ccccc23)c1 ; c1ccc(-c2ccccc2-c2c3ccccc3c(-c3cccc(-c4ccc5c(c4)sc4c6ccccc6ccc54)c3)c3ccccc23)cc1 ; c1ccc(-c2cccc(-c3c4ccccc4c(-c4cccc(-c5ccc6c(c5)sc5c7ccccc7ccc65)c4)c4ccccc34)c2)cc1]

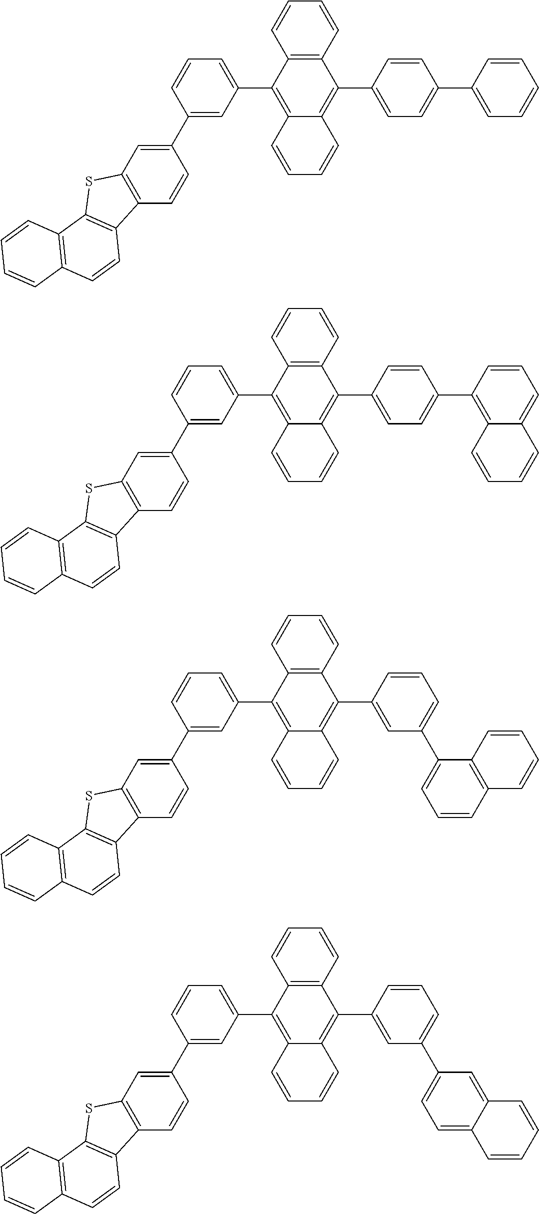 [SMILES: c1ccc(-c2ccc(-c3c4ccccc4c(-c4cccc(-c5ccc6c(c5)sc5c7ccccc7ccc65)c4)c4ccccc34)cc2)cc1 ; c1cc(-c2ccc3c(c2)sc2c4ccccc4ccc32)cc(-c2c3ccccc3c(-c3ccc(-c4cccc5ccccc45)cc3)c3ccccc23)c1 ; c1cc(-c2ccc3c(c2)sc2c4ccccc4ccc32)cc(-c2c3ccccc3c(-c3cccc(-c4cccc5ccccc45)c3)c3ccccc23)c1 ; c1cc(-c2ccc3ccccc3c2)cc(-c2c3ccccc3c(-c3cccc(-c4ccc5c(c4)sc4c6ccccc6ccc54)c3)c3ccccc23)c1]

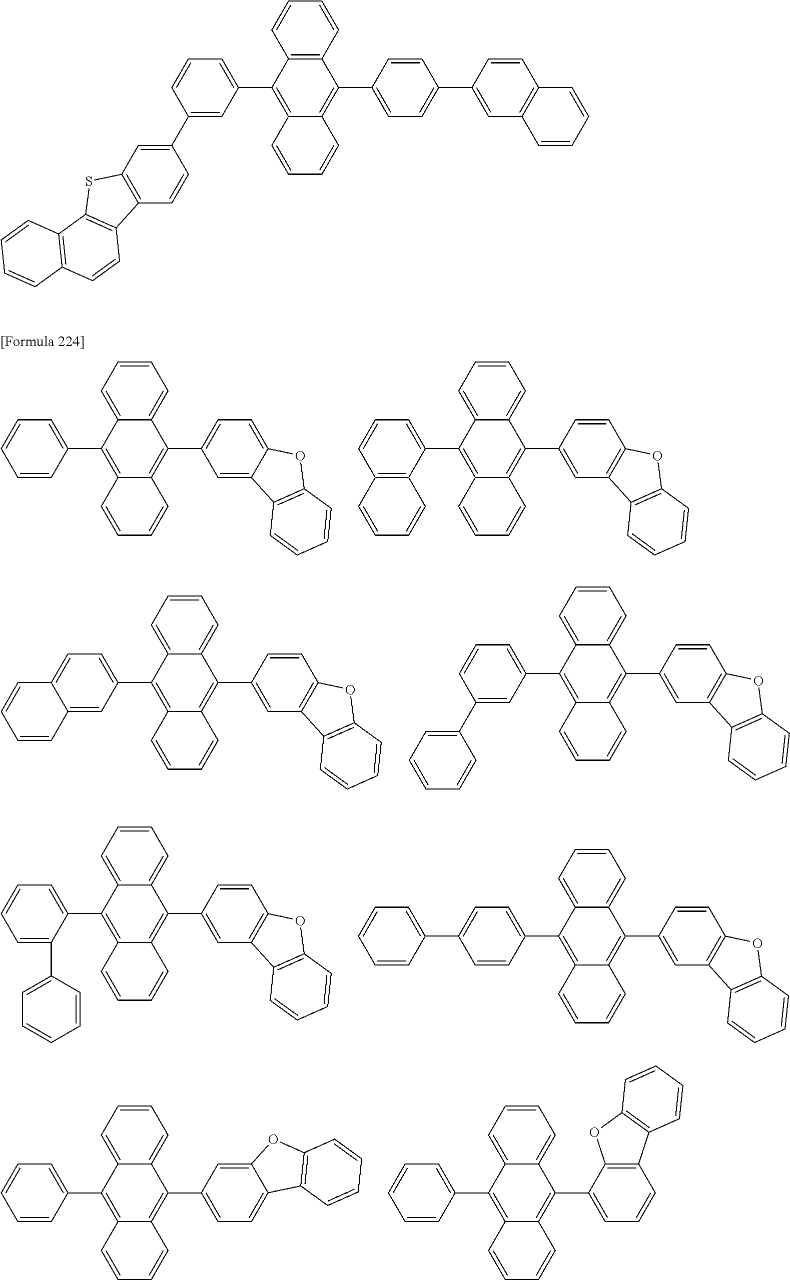

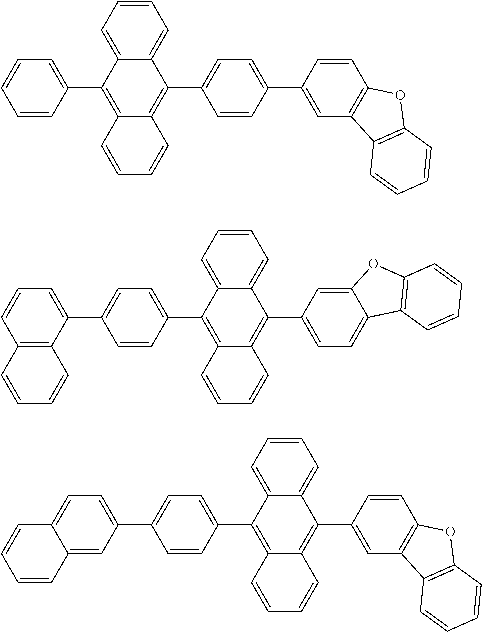

### Second Compound

The second compound is a fluorescent compound. A fluorescent compound is capable of emitting in a singlet state. The second compound may emit any color of fluorescence.

The second compound for the organic EL device of the first exemplary embodiment preferably emits blue fluorescence.

The second compound is preferably a compound represented by a formula (21) below.

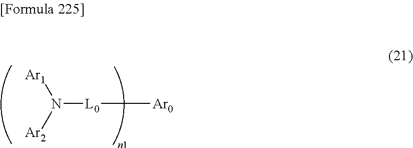

In the formula (21): n1 is an integer of 1 or more; Ar₀ is a group having a benzofluorene skeleton, fluoranthene skeleton, pyrene skeleton or chrysene skeleton; Ar₁ and Ar₂ are each independently a substituent selected from the group consisting of a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, and a substituted or unsubstituted aralkyl group having 7 to 30 carbon atoms; Ar₁ and Ar₂ may be bonded to form a saturated or unsaturated ring; when n1 is 2 or more, plural Ar₁ are mutually the same or different and plural Ar₂ are mutually the same or different;

L₀ is a single bond or a linking group; L₀ in a form of the linking group is selected from the group consisting of a substituted or unsubstituted aromatic hydrocarbon group having 6 to 30 ring carbon atoms and a substituted or unsubstituted heterocyclic group having 5 to 30 ring atoms; and when n1 is 2 or more, plural L₀ are mutually the same or different.

In the first exemplary embodiment, Ar₀ in the formula (21) is preferably a group having a pyrene skeleton or a chrysene skeleton.

Further, in the first exemplary embodiment, when n1 in the formula (21) is 2, L₀ are both preferably single bonds.

When Ar₀ is a pyrene skeleton, n1 is 2, and L₀ is a single bond, the nitrogen atom in the formula (21) is preferably bonded to position 3 or 8 of the pyrene skeleton. When Ar₀ is a chrysene skeleton, n1 is 2, and L₀ is a single bond, the nitrogen atom in the formula (21) is preferably bonded to position 6 or 12 of the chrysene skeleton.

In the first exemplary embodiment, the second compound is also preferably a compound represented by a formula (23) below.

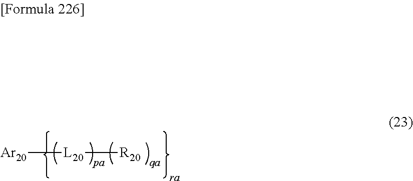

In the formula (23): pa is an integer of 0 to 5; qa and ra are each an integer of 1 to 5; Ar₂₀ is a group having a benzofluorene skeleton, fluoranthene skeleton, pyrene skeleton or chrysene skeleton; R₂₀ is a substituent selected from the group consisting of a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, and a substituted or unsubstituted aralkyl group having 7 to 30 carbon atoms; plural R₂₀ are mutually the same or different; R₂₀ may be bonded together to form a saturated or unsaturated ring; when pa is 0, Ar₂₀ and R₂₀ are bonded by a single bond; when pa is an integer of 1 to 5, L₂₀ is a linking group selected from the group consisting of a substituted or unsubstituted aromatic hydrocarbon group having 6 to 30 ring carbon atoms and a substituted or unsubstituted heterocyclic group having 5 to 30 ring atoms; plural L₂₀ are mutually the same or different; and L₂₀ may be bonded together to form a saturated or unsaturated ring.

Examples of the bonding pattern of the compound represented by the formula (23) include bonding patterns (23A) to (23K) below.

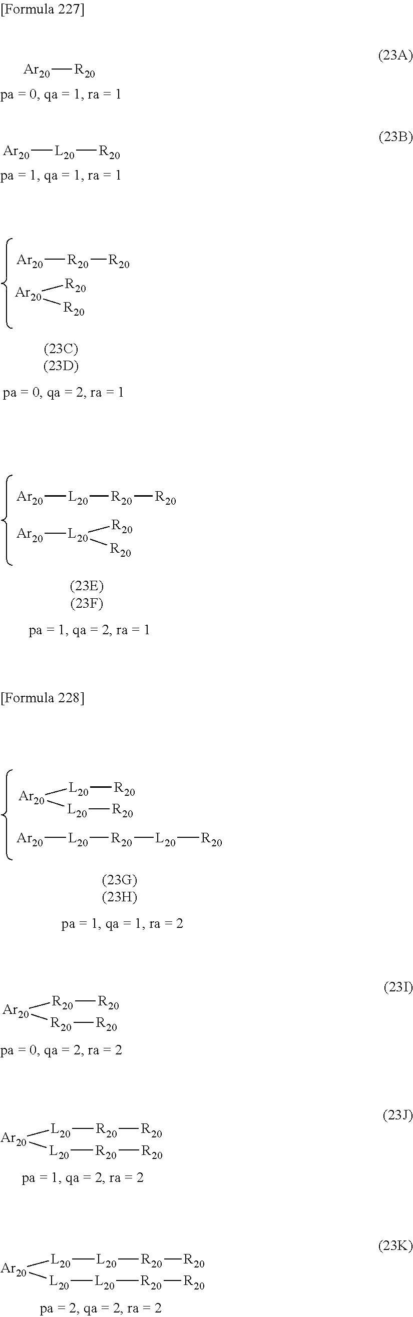

### Film Thickness of Emitting Layer

The emitting layer 7 in the organic EL device 1 of the first exemplary embodiment has a film thickness preferably ranging from 5 nm to 100 nm, more preferably from 7 nm to 100 nm, further preferably from 10 nm to 100 nm. The emitting layer 7 having a film thickness of 5 nm or more is easily formable and easily adjustable in chromaticity. The emitting layer 7 having a film thickness of 100 nm or less enables prevention of a rise in the drive voltage.

### Contents of Compounds in Emitting Layer

The emitting layer 7 in the organic EL device 1 of the first exemplary embodiment preferably contains the first compound at a rate ranging from 50 mass % to 99 mass %, more preferably from 85 mass % to 99 mass %. The content of the second compound preferably ranges from 1 mass % to 50 mass %, more preferably 1 mass % to 15 mass %. It should be noted that the emitting layer 7 of the first exemplary embodiment may contain a material other than the first compound and the second compound.

### Hole Transporting Layer

The hole transporting layer 6 in the organic EL device 1 contains a third compound represented by a formula (3) below.

### Third Compound

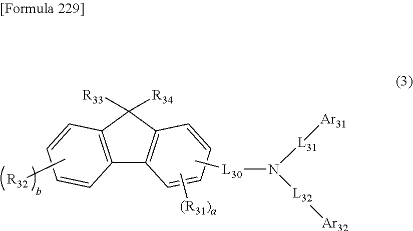

In the formula (3), R₃₁ to R₃₂ are each independently a hydrogen atom or a substituent. R₃₁ to R₃₂ in a form of the substituents are each independently selected from the group consisting of the substituents listed for R₁₁ to R₁₈ in a form of the substituents.

a is 3 and plural R₃₁ may be mutually the same or different. R₃₁ in a form of the substituents may be bonded together to form a cyclic structure.

b is 4 and plural R₃₂ may be mutually the same or different. R₃₂ in a form of the substituents may be bonded together to form a cyclic structure.

R₃₃ to R₃₄ are each independently a hydrogen atom or a substituent. R₃₃ to R₃₄ in a form of the substituents are each independently selected from the group consisting of a substituted or unsubstituted alkyl group having 1 to 20 carbon atoms, substituted or unsubstituted fluoroalkyl group having 1 to 20 carbon atoms, substituted or unsubstituted aromatic hydrocarbon group having 6 to 50 ring carbon atoms, substituted or unsubstituted heterocyclic group having 3 to 50 ring atoms, and cyano group. R₃₃ and R₃₄ in a form of the substituents may be bonded together to form a cyclic structure.

L₃₀, L₃₁ and L₃₂ are each independently a single bond or a linking group. L₃₀, L₃₁ and L₃₂ in a form of the linking groups are each independently a substituted or unsubstituted aromatic hydrocarbon group having 6 to 30 ring carbon atoms or a substituted or unsubstituted heterocyclic group having 5 to 30 ring atoms.

Ar₃₁ and Ar₃₂ are each independently a substituted or unsubstituted aromatic hydrocarbon group having 6 to 30 ring carbon atoms or a substituted or unsubstituted heterocyclic group having 5 to 30 ring atoms.

R₃₃ and R₃₄ are preferably each independently a substituted or unsubstituted aromatic hydrocarbon group having 6 to 30 ring carbon atoms, more preferably a substituted or unsubstituted aromatic hydrocarbon group having 6 to 20 ring carbon atoms, further preferably a substituted or unsubstituted aromatic hydrocarbon group having 6 to 14 ring carbon atoms, still further preferably a substituted or unsubstituted aromatic hydrocarbon group having 6 to 12 ring carbon atoms.

The third compound is also preferably represented by a formula (30) below.

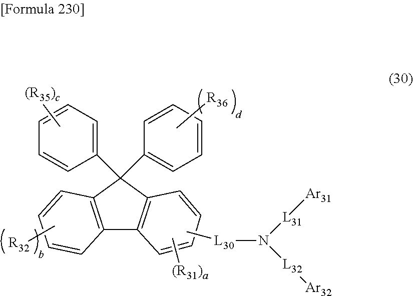

In the formula (30), R₃₁, R₃₂, a, b, Ar₃₁, Ar₃₂, L₃₀, L₃₁ and L₃₂ respectively represent the same as R₃₁, R₃₂, a, b, Ar₃₁, Ar₃₂, L₃₀, L₃₁ and L₃₂ in the formula (3). R₃₅ and R₃₆ are each independently a hydrogen atom or a substituent. R₃₅ and R₃₆ in a form of the substituents are each independently selected from the group consisting of the substituents listed for R₁ to R₈ in a form of the substituents. c is 5, plural R₃₅ may be mutually the same or different, and R₃₅ in a form of the substituents may be bonded together to form a cyclic structure. d is 5, plural R₃₆ may be mutually the same or different, and R₃₆ in a form of the substituents may be bonded together to form a cyclic structure.

The third compound may have a structure where the aromatic ring bonded to R₃₅ is single-bonded to the aromatic ring bonded to R₃₆ in the formula (30). For instance, the third compound preferably has a spirofluorene ring.

Moreover, the third compound preferably has no structure where the aromatic ring bonded to R₃₅ is bonded to the aromatic ring bonded to R₃₆ in the formula (30). For instance, the third compound preferably has a 9,9-diphenylfluorene ring.

The third compound is also preferably represented by a formula (31) below.

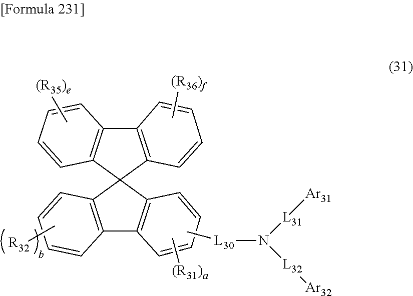

In the formula (31), R₃₁ to R₃₂, a, b, L₃₀ to L₃₂, Ar₃₁ and Ar₃₂ each independently represent the same as R₃₁ to R₃₂, a, b, L₃₀ to L₃₂, Ar₃₁ and Ar₃₂ in the formula (3). R₃₅ and R₃₆ are each independently a hydrogen atom or a substituent. R₃₅ and R₃₆ in a form of the substituents are each independently selected from the group consisting of the substituents listed for R₁ to R₈ in a form of the substituents. Each of e and f is 4. Plural R₃₅ may be mutually the same or different, and R₃₅ in a form of the substituents may be bonded together to form a cyclic structure. Plural R₃₆ may be mutually the same or different, and R₃₆ in a form of the substituents may be bonded together to form a cyclic structure.

The third compound is also preferably represented by a formula (32) below.

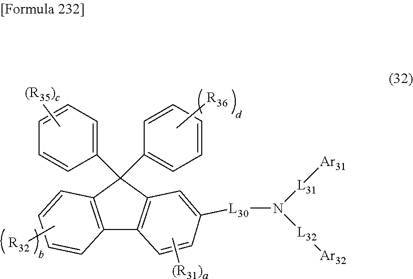

In the formula (32), R₃₁ to R₃₂, a, b, L₃₀ to L₃₂, Ar₃₁ and Ar₃₂ each independently represent the same as R₃₁ to R₃₂, a, b, L₃₀ to L₃₂, Ar₃₁ and Ar₃₂ in the formula (3). R₃₅ and R₃₆ are each independently a hydrogen atom or a substituent. R₃₅ and R₃₆ in a form of the substituents are each independently selected from the group consisting of the substituents listed for R₁ to R₈ in a form of the substituents. c is 5 and d is 5. Plural R₃₅ may be mutually the same or different, and R₃₅ in a form of the substituents may be bonded together to form a cyclic structure. Plural R₃₆ may be mutually the same or different, and R₃₆ in a form of the substituents may be bonded together to form a cyclic structure.

Ar₃₁ and Ar₃₂ are preferably each independently one selected from the group consisting of groups represented by formulae (31a) to (31k) and (31m) below.

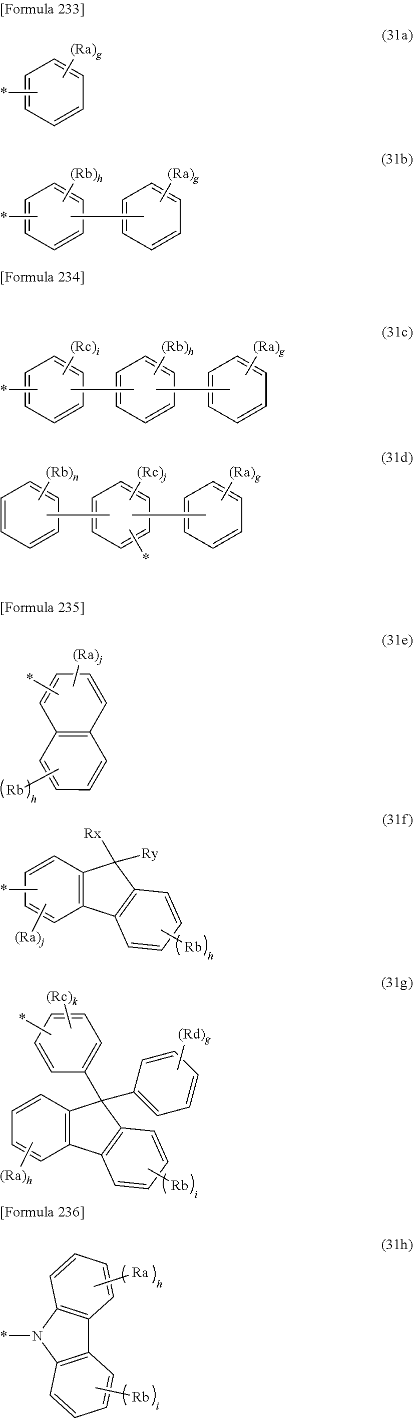

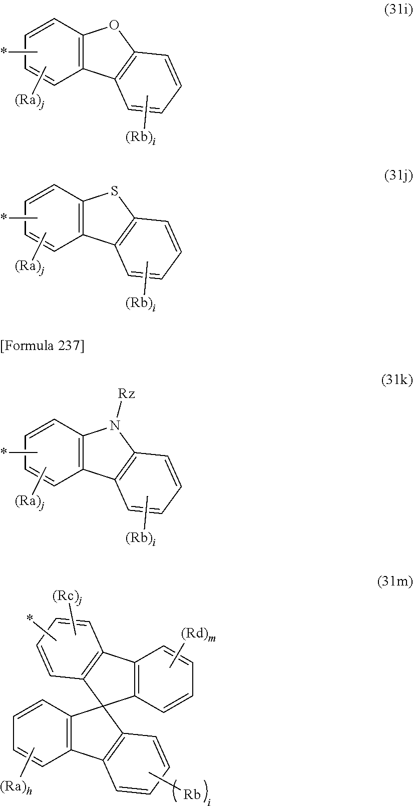

In the formulae (31a) to (31k) and (31m), Ra, Rb, Re and Rd are each independently a hydrogen atom or a substituent, and Ra, Rb, Re and Rd in a form of the substituents are each independently selected from the group consisting of the substituents listed for R₁ to R₈ in a form of the substituents.

Rx and Ry are each independently a hydrogen atom or a substituent. Rx and Ry in a form of the substituents are each selected from the group consisting of a substituted or unsubstituted alkyl group having 1 to 30 carbon atoms and a substituted or unsubstituted aromatic hydrocarbon group having 6 to 30 ring carbon atoms.

Rz is a hydrogen atom or a substituent. Rz in a form of the substituent is selected from the group consisting of a substituted or unsubstituted aromatic hydrocarbon group having 6 to 30 ring carbon atoms and a substituted or unsubstituted heterocyclic group having 5 to 30 ring atoms.

g is 5, h is 4, i is 4, j is 3, k is 4, m is 4, and n is 5.

Plural Ra may be mutually the same or different. Ra in a form of the substituents may be bonded together to form a cyclic structure.

Plural Rb may be mutually the same or different. Rb in a form of the substituents may be bonded together to form a cyclic structure.

Plural Rc may be mutually the same or different. Rc in a form of the substituents may be bonded together to form a cyclic structure.

Plural Rd may be mutually the same or different. Rd in a form of the substituents may be bonded together to form a cyclic structure.

* represents a position where L₃₁ or L₃₂ is bonded.

Ar₃₁ and Ar₃₂ are preferably each independently one selected from the group consisting of groups represented by formulae (31n) and (31p) to (31w) below.

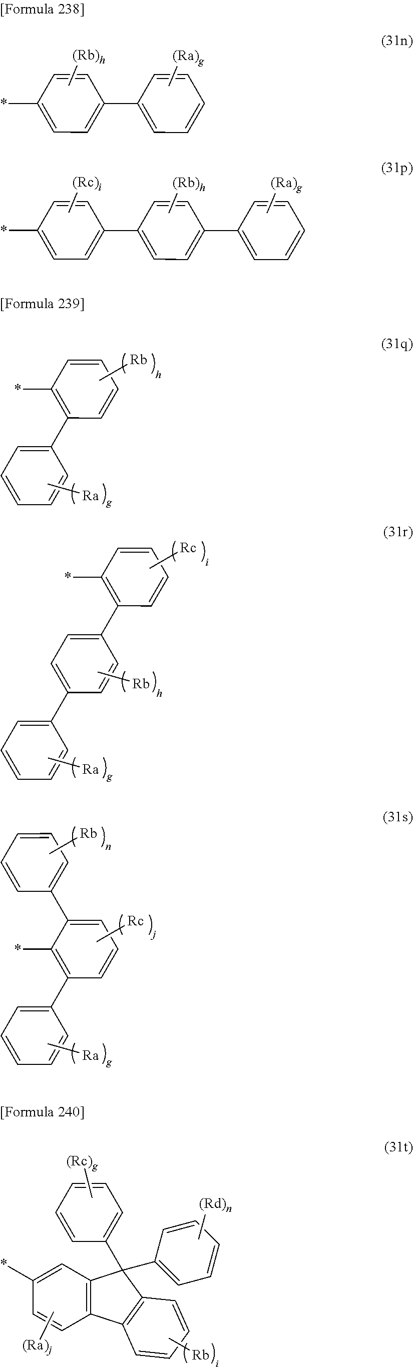

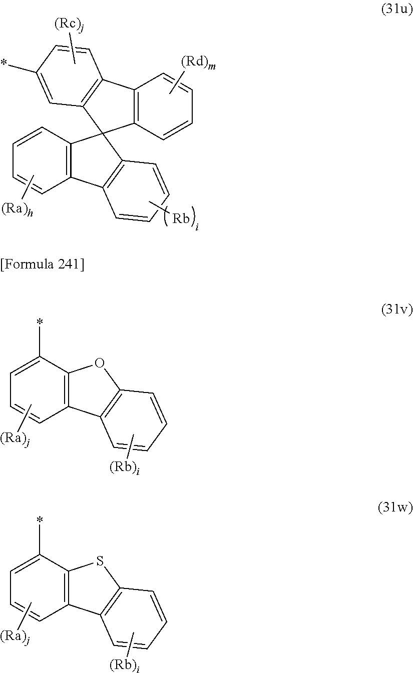

In the formulae (31n) and (31p) to (31w), Ra, Rb, Rc, Rd, g, h, i, j, m, n and * respectively represent the same as Ra, Rb, Rc, Rd, g, h, i, j, m, n and * in the formulae (31a) to (31k) and (31m).

Ra, Rb, Rc and Rd are each preferably a hydrogen atom.

Rx, Ry and Rz are each preferably a substituent

L₃₀, L₃₁ and L₃₂ are each independently a single bond or a linking group. L₃₀, L₃₁ and L₃₂ in a form of the linking groups are each independently a substituted or unsubstituted aromatic hydrocarbon group having 6 to 30 ring carbon atoms.

L₃₀, L₃₁ and L₃₂ in a form of the linking groups are preferably each independently a linking group selected from the group consisting of groups represented by formulae (3 ix) to (31 z) below.

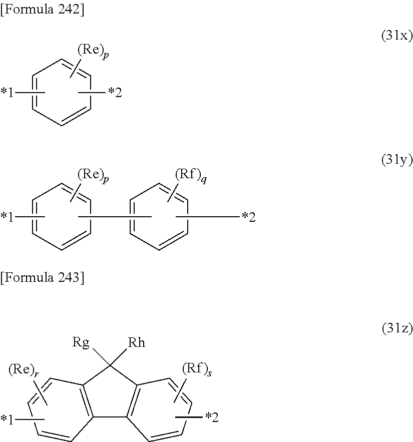

In the formulae (31x) to (31z), Re and Rf are each independently a hydrogen atom or a substituent. When in a form of the substituents, Re and Rf are each selected from the group consisting of the substituents listed for R₁ to R₈ in a form of the substituents.

Rg and Rh are each independently a hydrogen atom or a substituent. When in a form of the substituents, Rg and Rh are each selected from the group consisting of a substituted or unsubstituted alkyl group having 1 to 30 carbon atoms and a substituted or unsubstituted aromatic hydrocarbon group having 6 to 30 ring carbon atoms.

p is 4, q is 4, r is 3, and s is 3.

Plural Re may be mutually the same or different. Re in a form of the substituents may be bonded together to form a cyclic structure.

Plural Rf may be mutually the same or different. Rf in a form of the substituents may be bonded together to form a cyclic structure.

*1 and *2 each represent a position for bonding to a nitrogen atom, Ar₃₁, Ar₃₂ or the fluorene ring in the structure represented by the formula (3).

When L₃₀ is a single bond, the fluorene skeleton is directly bonded to a nitrogen atom to reduce the ionization potential of the third compound. The reduced ionization potential reduces an energy barrier against the anode 3 or the hole injecting layer 5, facilitating electron injection into the emitting layer 7 and, consequently, lowering the drive voltage of the organic EL device 1.

Each of groups represented by the formulae (31t) to (31u) is also preferably directly bonded to the nitrogen atom.

Specific examples of the third compound are given below. It should be noted that the compound of the invention is not limited to the examples.

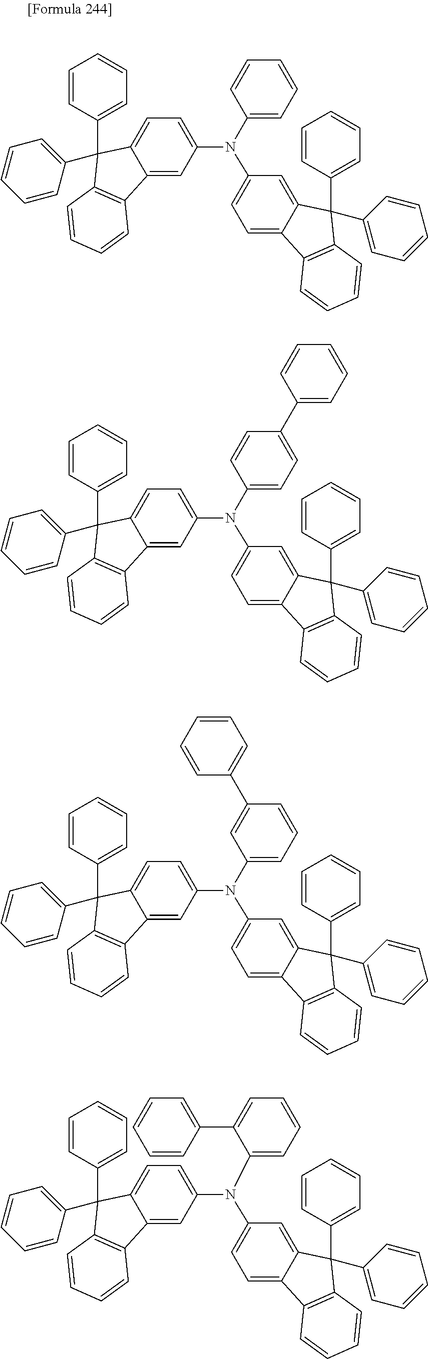

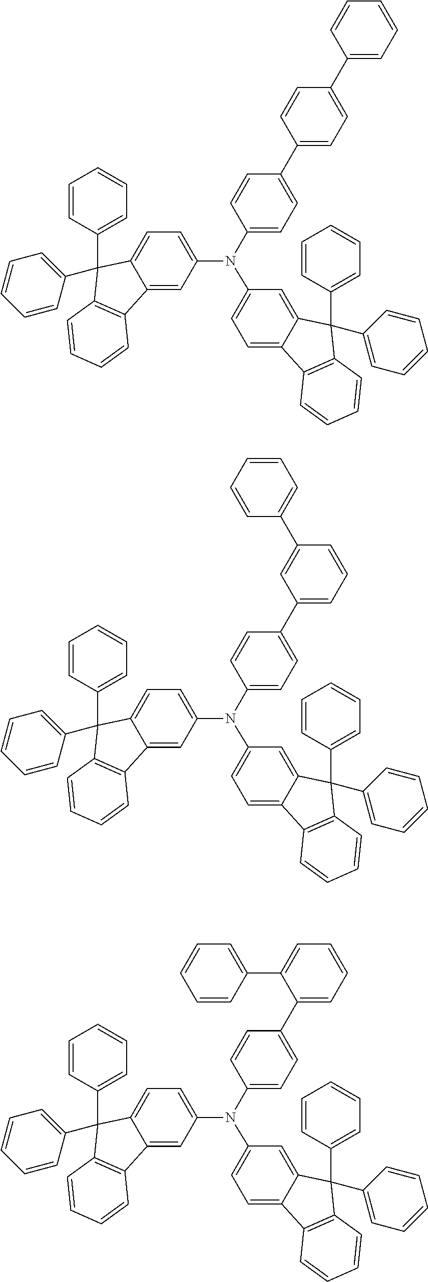 [SMILES: c1ccc(-c2ccc(-c3ccc(N(c4ccc5c(c4)-c4ccccc4C5(c4ccccc4)c4ccccc4)c4ccc5c(c4)C(c4ccccc4)(c4ccccc4)c4ccccc4-5)cc3)cc2)cc1 ; c1ccc(-c2cccc(-c3ccc(N(c4ccc5c(c4)-c4ccccc4C5(c4ccccc4)c4ccccc4)c4ccc5c(c4)C(c4ccccc4)(c4ccccc4)c4ccccc4-5)cc3)c2)cc1 ; c1ccc(-c2ccccc2-c2ccc(N(c3ccc4c(c3)-c3ccccc3C4(c3ccccc3)c3ccccc3)c3ccc4c(c3)C(c3ccccc3)(c3ccccc3)c3ccccc3-4)cc2)cc1]

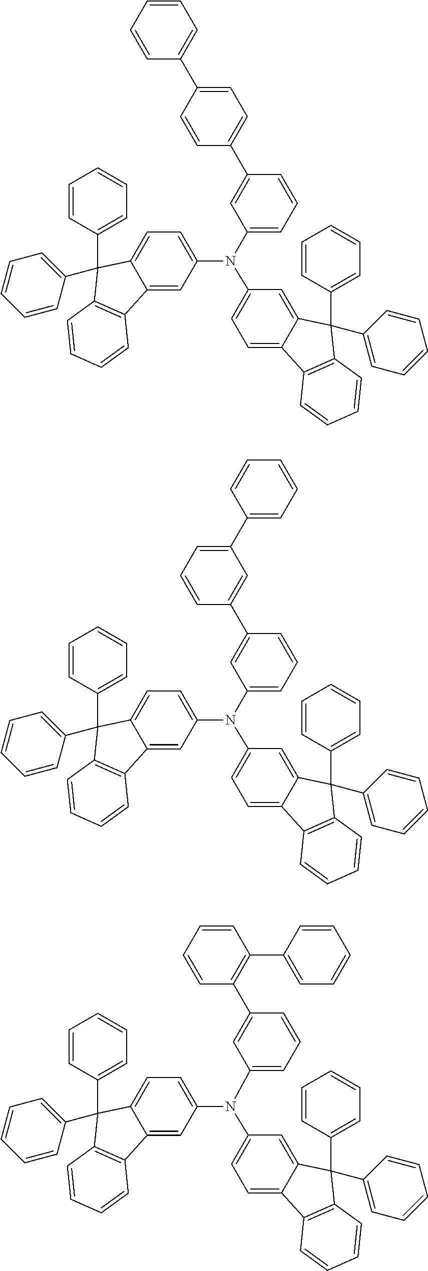

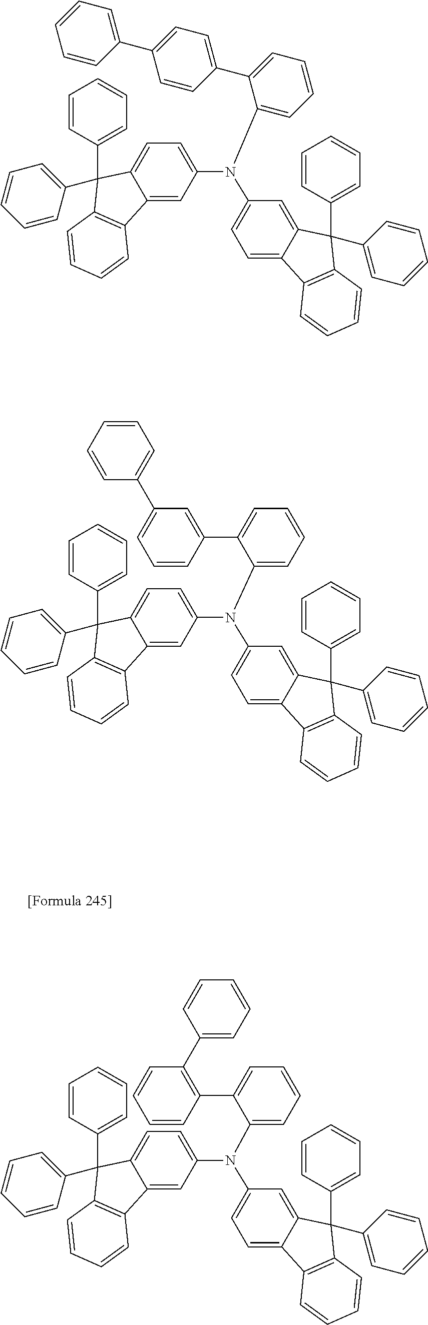 [SMILES: c1ccc(-c2ccc(-c3ccccc3N(c3ccc4c(c3)-c3ccccc3C4(c3ccccc3)c3ccccc3)c3ccc4c(c3)C(c3ccccc3)(c3ccccc3)c3ccccc3-4)cc2)cc1 ; c1ccc(-c2cccc(-c3ccccc3N(c3ccc4c(c3)-c3ccccc3C4(c3ccccc3)c3ccccc3)c3ccc4c(c3)C(c3ccccc3)(c3ccccc3)c3ccccc3-4)c2)cc1 ; c1ccc(-c2ccccc2-c2ccccc2N(c2ccc3c(c2)-c2ccccc2C3(c2ccccc2)c2ccccc2)c2ccc3c(c2)C(c2ccccc2)(c2ccccc2)c2ccccc2-3)cc1]

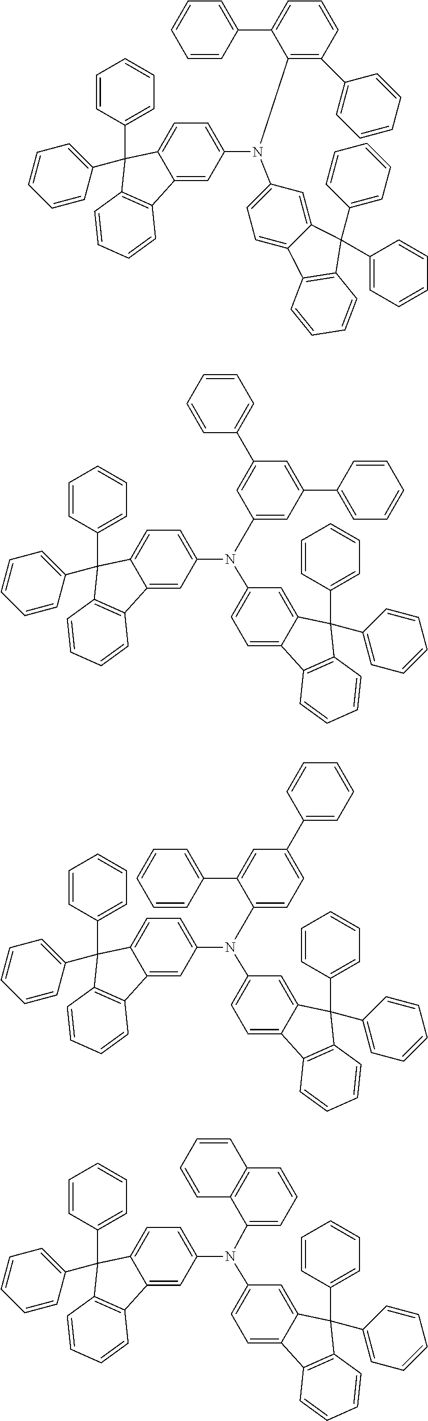

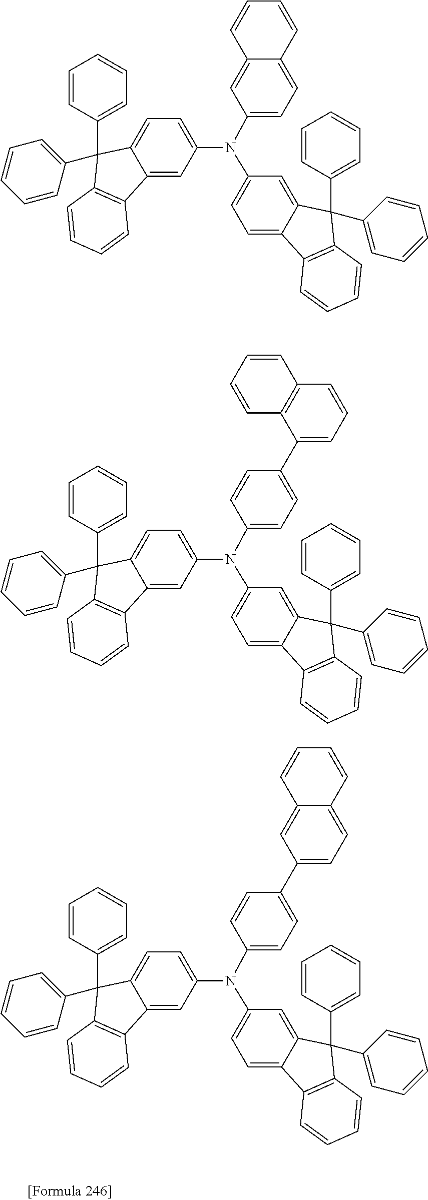

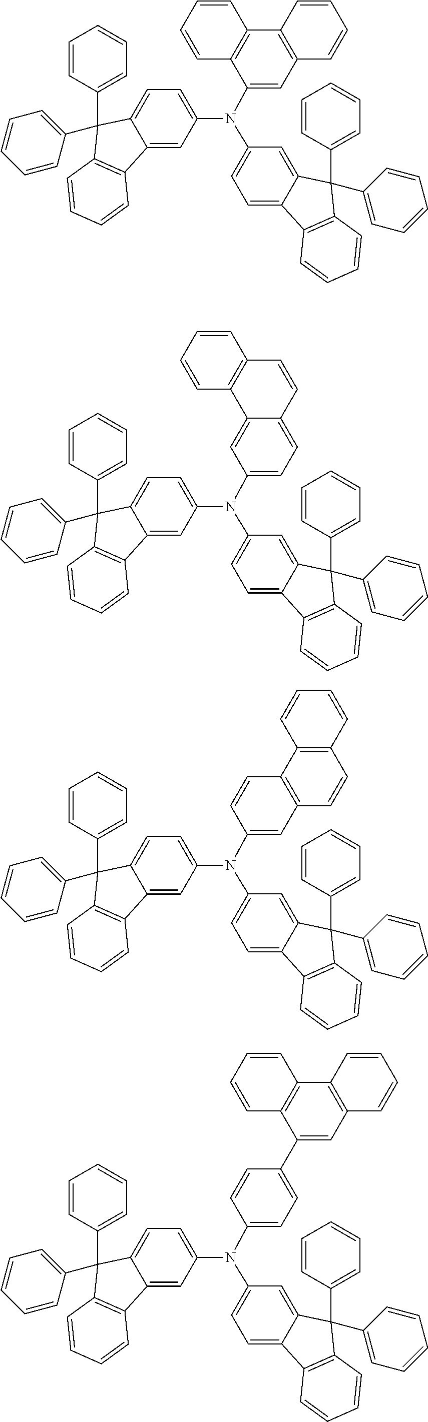

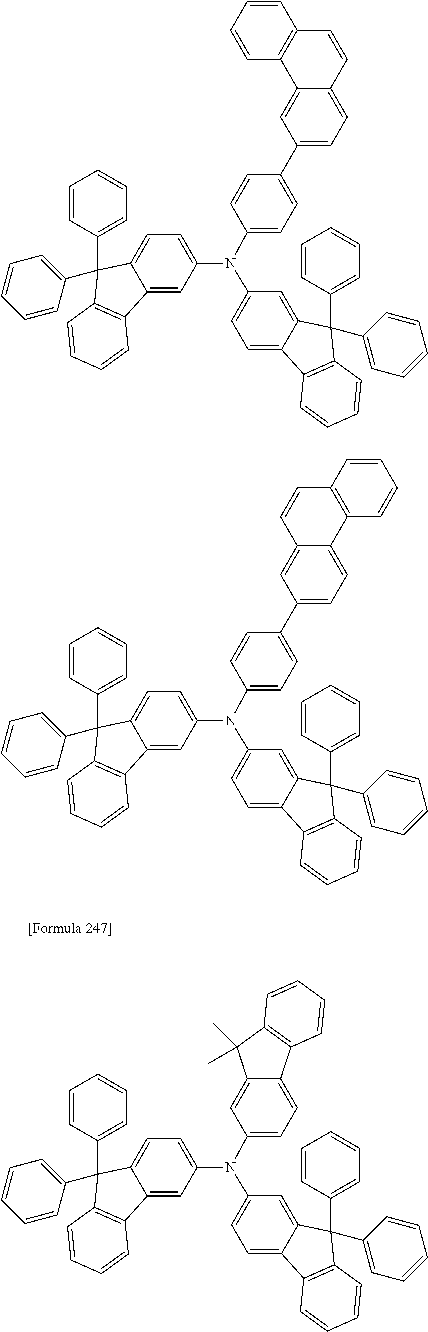

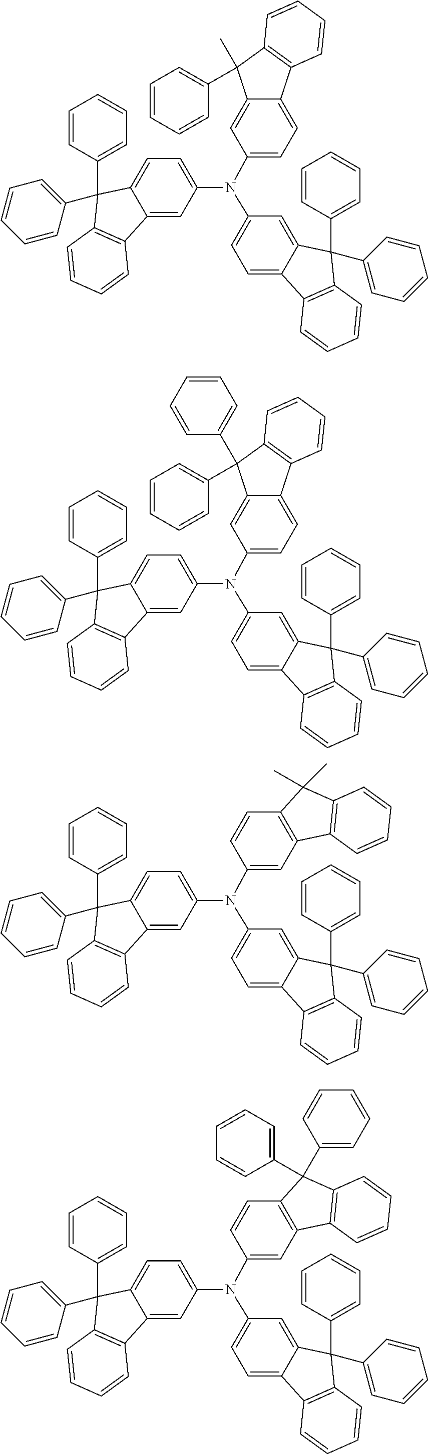

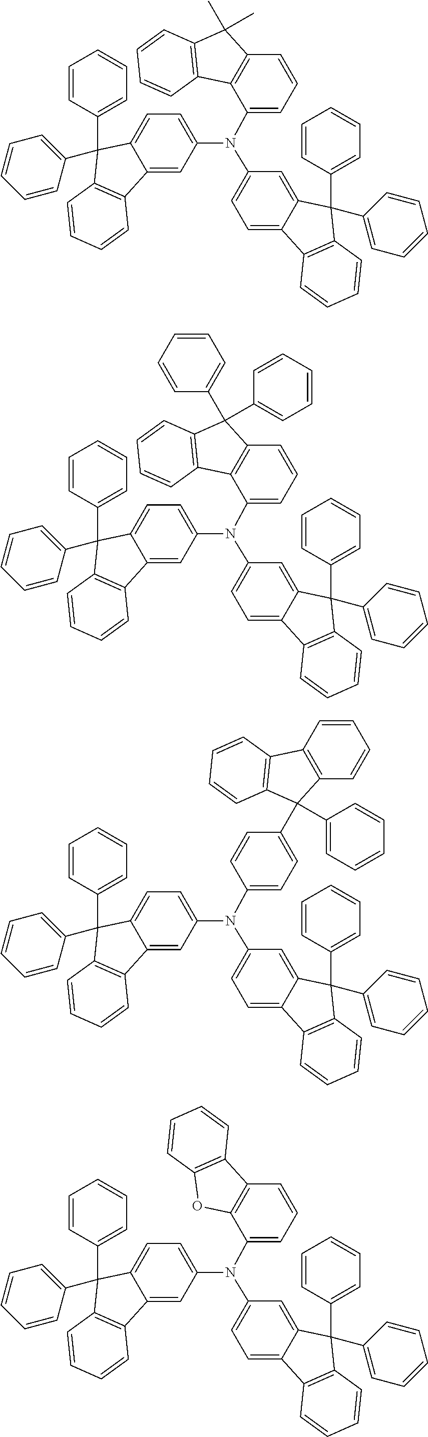

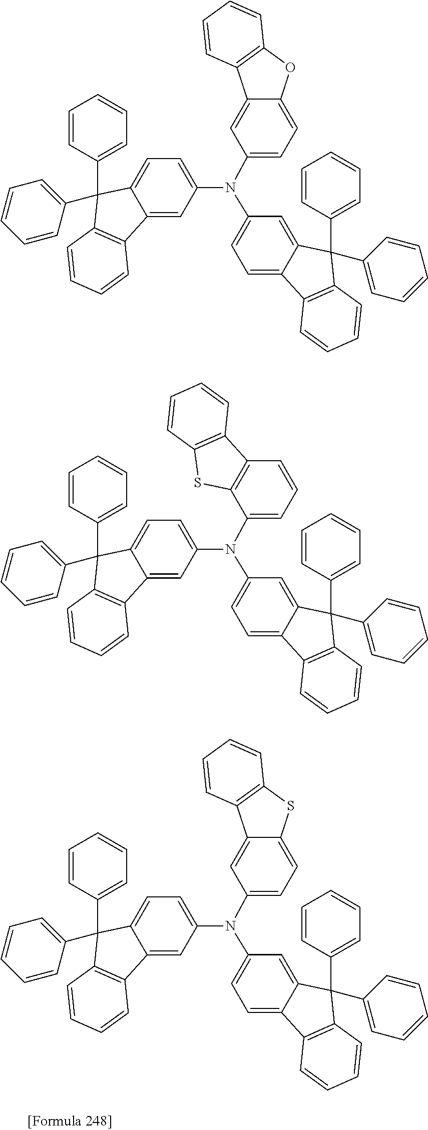 [SMILES: c1ccc(C2(c3ccccc3)c3ccccc3-c3cc(N(c4ccc5c(c4)C(c4ccccc4)(c4ccccc4)c4ccccc4-5)c4ccc5oc6ccccc6c5c4)ccc32)cc1 ; c1ccc(C2(c3ccccc3)c3ccccc3-c3cc(N(c4ccc5c(c4)C(c4ccccc4)(c4ccccc4)c4ccccc4-5)c4cccc5c4sc4ccccc45)ccc32)cc1 ; c1ccc(C2(c3ccccc3)c3ccccc3-c3cc(N(c4ccc5c(c4)C(c4ccccc4)(c4ccccc4)c4ccccc4-5)c4ccc5sc6ccccc6c5c4)ccc32)cc1]

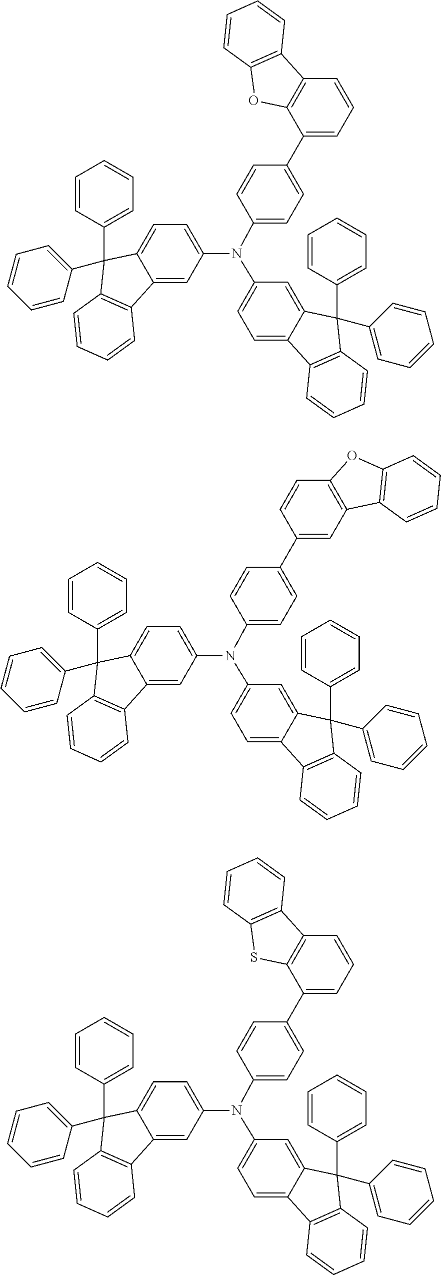

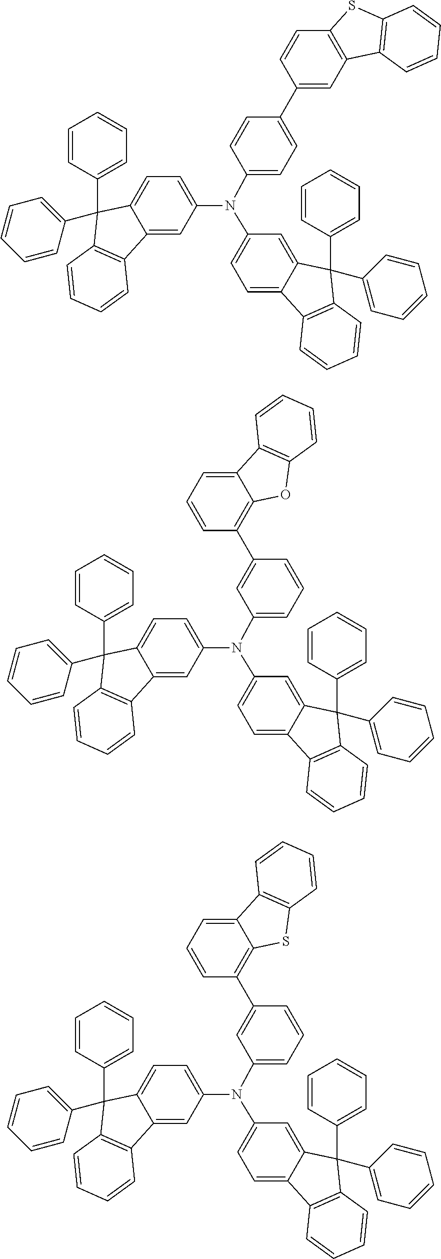

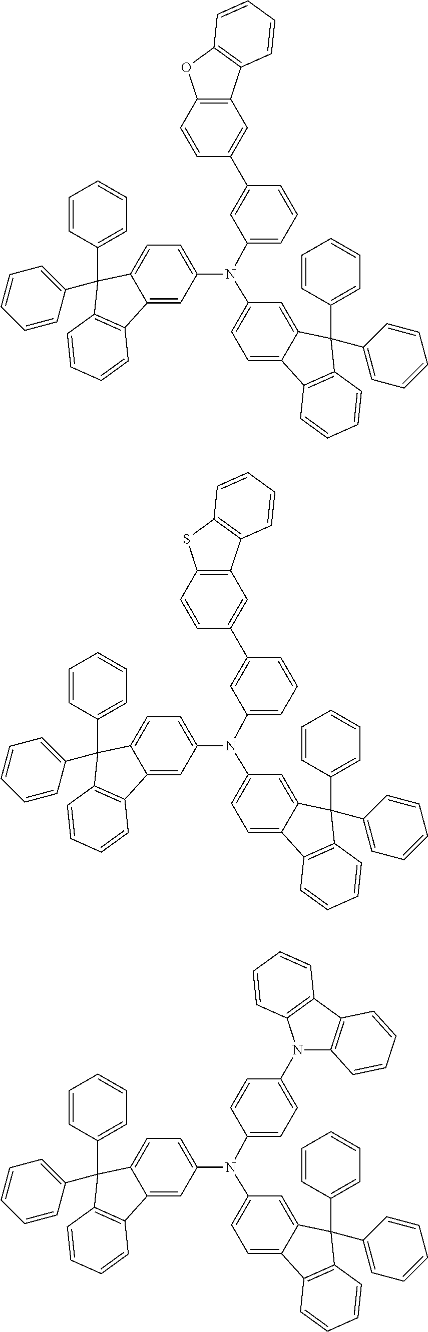 [SMILES: c1ccc(C2(c3ccccc3)c3ccccc3-c3cc(N(c4cccc(-c5ccc6oc7ccccc7c6c5)c4)c4ccc5c(c4)C(c4ccccc4)(c4ccccc4)c4ccccc4-5)ccc32)cc1 ; c1ccc(C2(c3ccccc3)c3ccccc3-c3cc(N(c4cccc(-c5ccc6sc7ccccc7c6c5)c4)c4ccc5c(c4)C(c4ccccc4)(c4ccccc4)c4ccccc4-5)ccc32)cc1 ; c1ccc(C2(c3ccccc3)c3ccccc3-c3cc(N(c4ccc(-n5c6ccccc6c6ccccc65)cc4)c4ccc5c(c4)C(c4ccccc4)(c4ccccc4)c4ccccc4-5)ccc32)cc1]

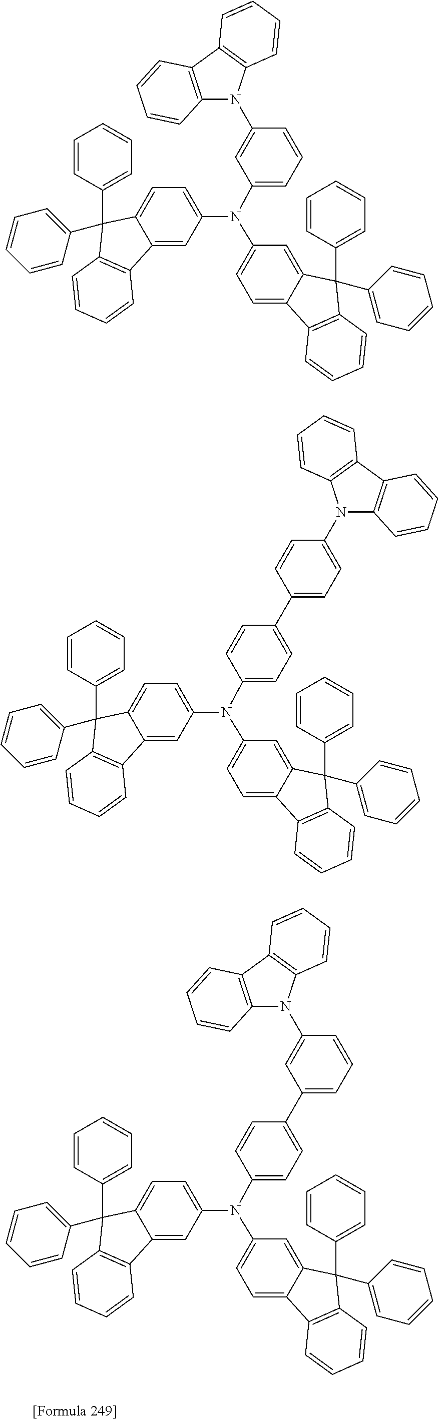

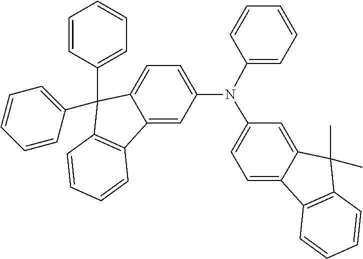 [SMILES: CC1(C)c2ccccc2-c2ccc(N(c3ccccc3)c3ccc4c(c3)-c3ccccc3C4(c3ccccc3)c3ccccc3)cc21]

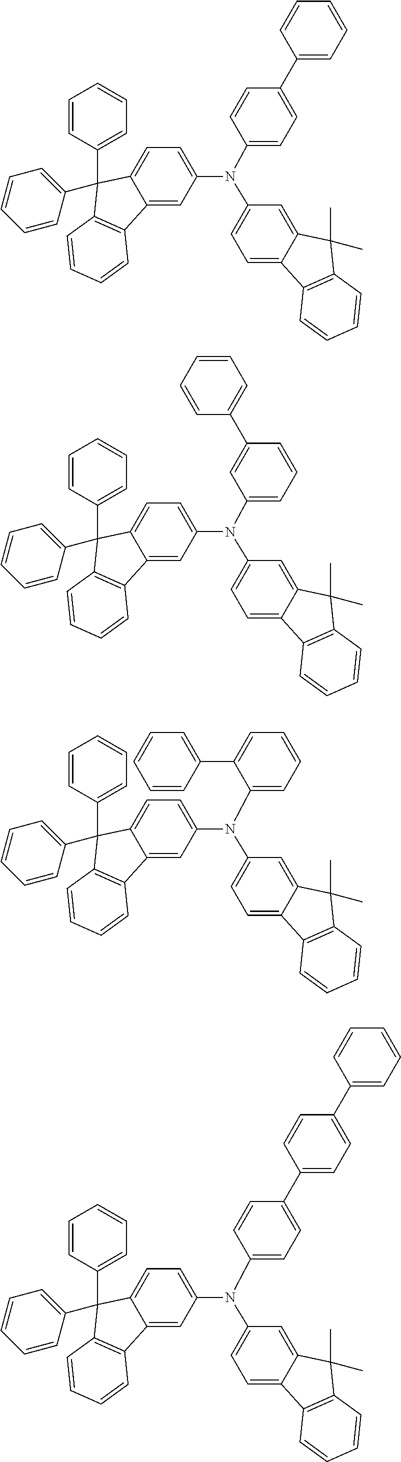

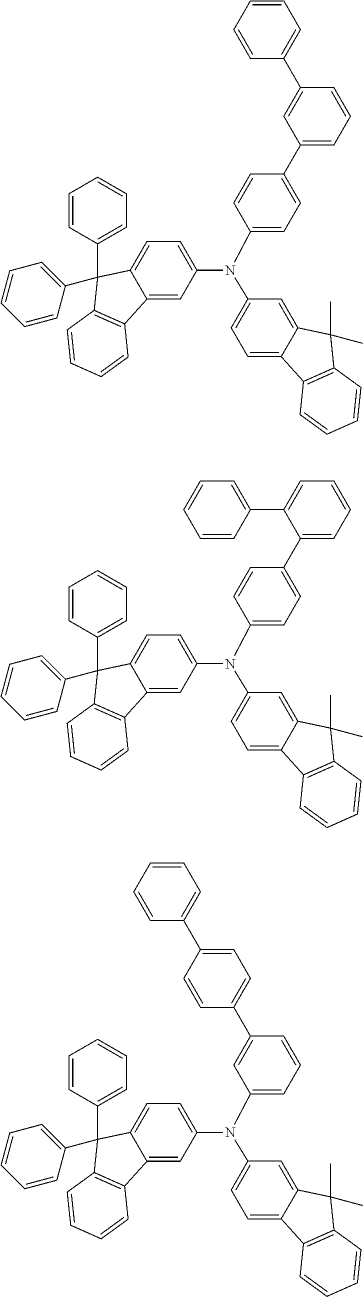 [SMILES: CC1(C)c2ccccc2-c2ccc(N(c3ccc(-c4cccc(-c5ccccc5)c4)cc3)c3ccc4c(c3)-c3ccccc3C4(c3ccccc3)c3ccccc3)cc21 ; CC1(C)c2ccccc2-c2ccc(N(c3ccc(-c4ccccc4-c4ccccc4)cc3)c3ccc4c(c3)-c3ccccc3C4(c3ccccc3)c3ccccc3)cc21 ; CC1(C)c2ccccc2-c2ccc(N(c3cccc(-c4ccc(-c5ccccc5)cc4)c3)c3ccc4c(c3)-c3ccccc3C4(c3ccccc3)c3ccccc3)cc21]

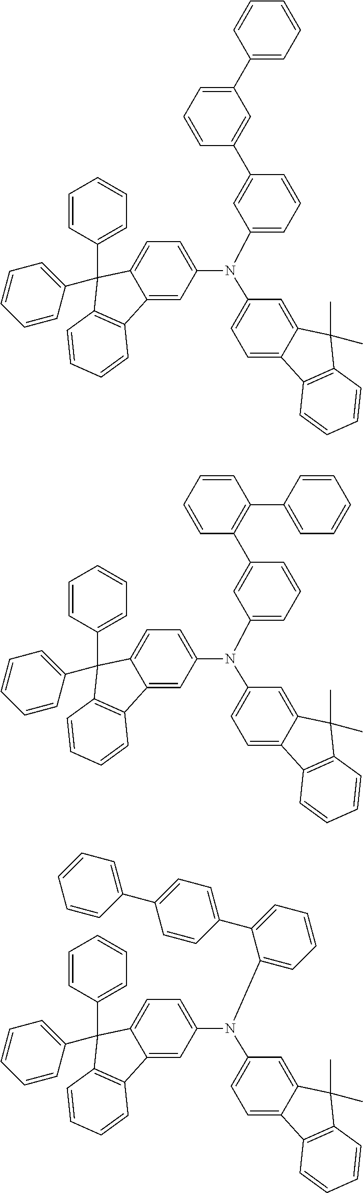

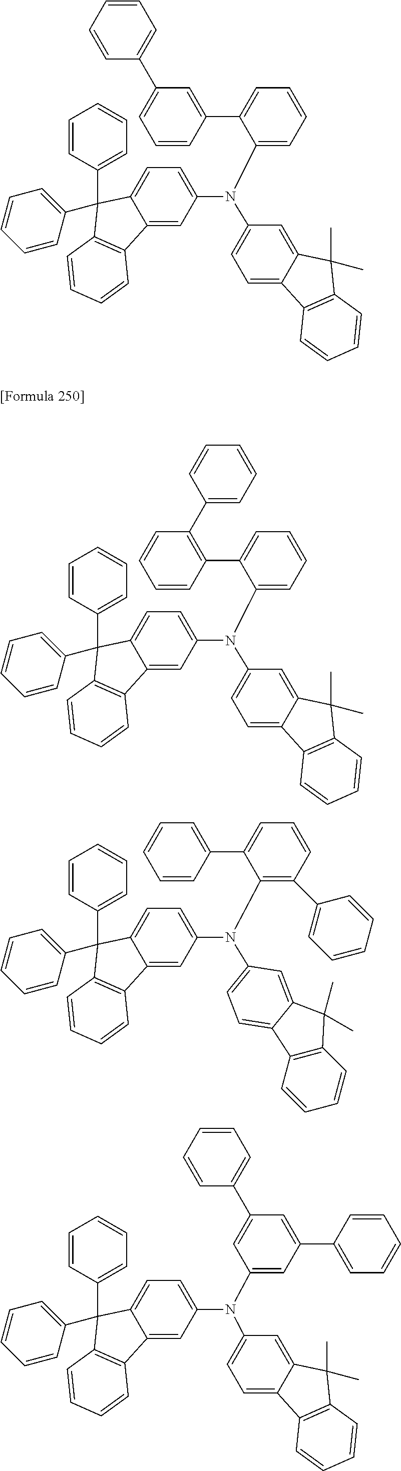

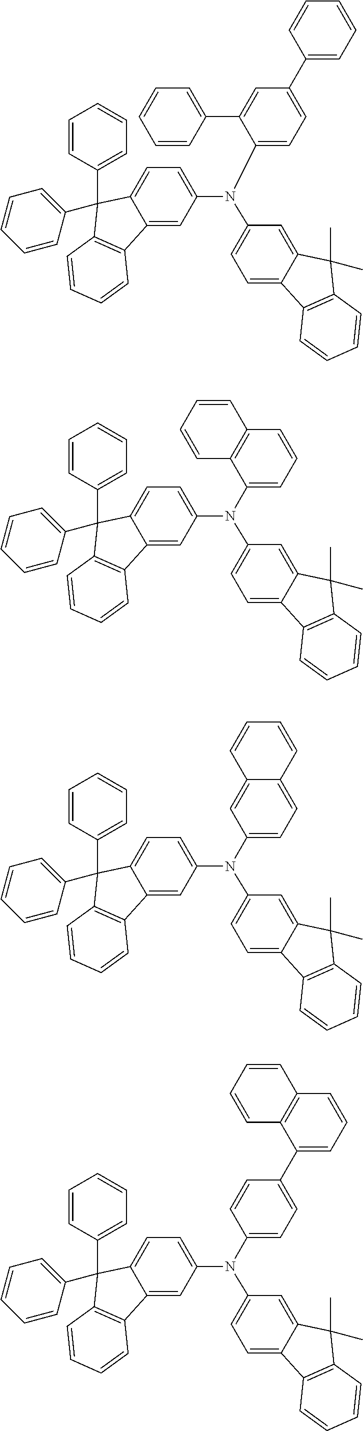 [SMILES: CC1(C)c2ccccc2-c2ccc(N(c3ccc4c(c3)-c3ccccc3C4(c3ccccc3)c3ccccc3)c3ccc(-c4ccccc4)cc3-c3ccccc3)cc21 ; CC1(C)c2ccccc2-c2ccc(N(c3ccc4c(c3)-c3ccccc3C4(c3ccccc3)c3ccccc3)c3cccc4ccccc34)cc21 ; CC1(C)c2ccccc2-c2ccc(N(c3ccc4c(c3)-c3ccccc3C4(c3ccccc3)c3ccccc3)c3ccc4ccccc4c3)cc21 ; CC1(C)c2ccccc2-c2ccc(N(c3ccc(-c4cccc5ccccc45)cc3)c3ccc4c(c3)-c3ccccc3C4(c3ccccc3)c3ccccc3)cc21]

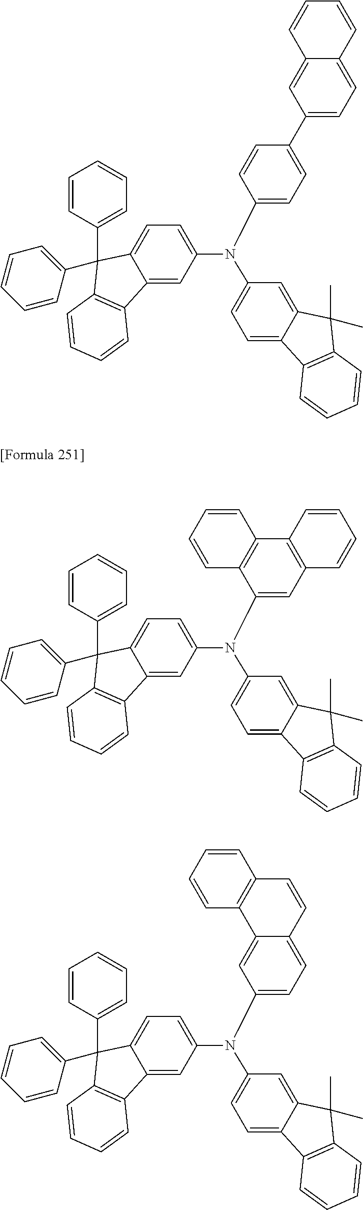 [SMILES: CC1(C)c2ccccc2-c2ccc(N(c3ccc(-c4ccc5ccccc5c4)cc3)c3ccc4c(c3)-c3ccccc3C4(c3ccccc3)c3ccccc3)cc21 ; CC1(C)c2ccccc2-c2ccc(N(c3ccc4c(c3)-c3ccccc3C4(c3ccccc3)c3ccccc3)c3cc4ccccc4c4ccccc34)cc21 ; CC1(C)c2ccccc2-c2ccc(N(c3ccc4c(c3)-c3ccccc3C4(c3ccccc3)c3ccccc3)c3ccc4ccc5ccccc5c4c3)cc21]

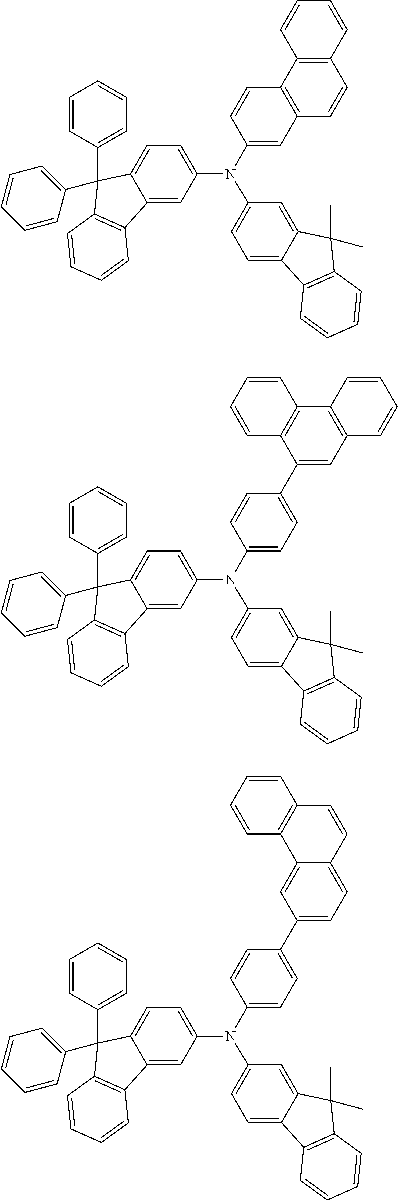

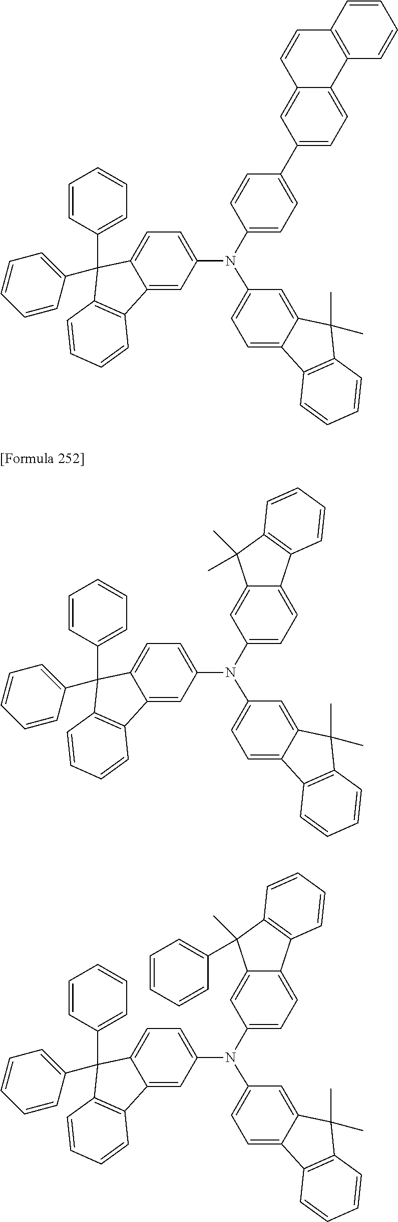 [SMILES: CC1(C)c2ccccc2-c2ccc(N(c3ccc(-c4ccc5c(ccc6ccccc65)c4)cc3)c3ccc4c(c3)-c3ccccc3C4(c3ccccc3)c3ccccc3)cc21 ; CC1(C)c2ccccc2-c2ccc(N(c3ccc4c(c3)-c3ccccc3C4(c3ccccc3)c3ccccc3)c3ccc4c(c3)C(C)(C)c3ccccc3-4)cc21 ; CC1(C)c2ccccc2-c2ccc(N(c3ccc4c(c3)-c3ccccc3C4(c3ccccc3)c3ccccc3)c3ccc4c(c3)C(C)(c3ccccc3)c3ccccc3-4)cc21]

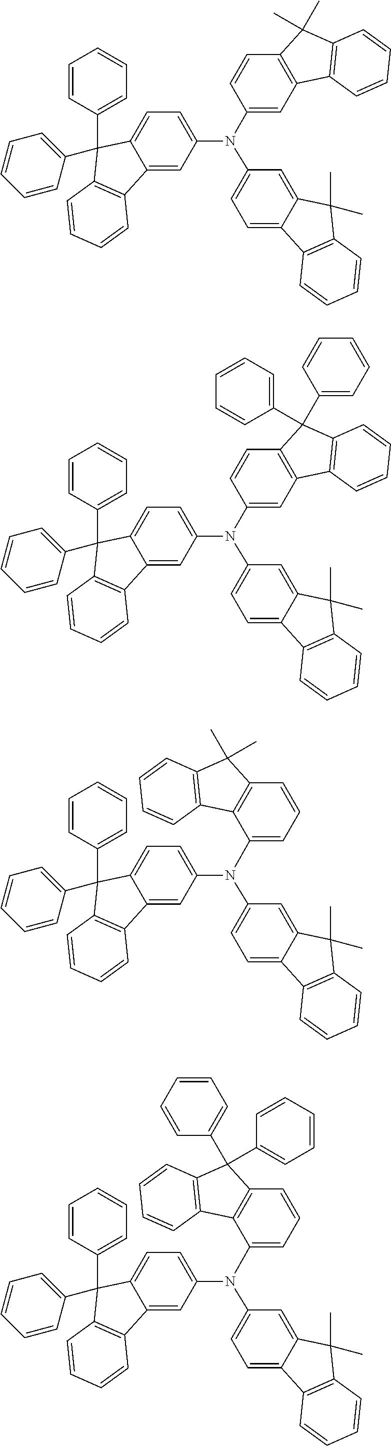

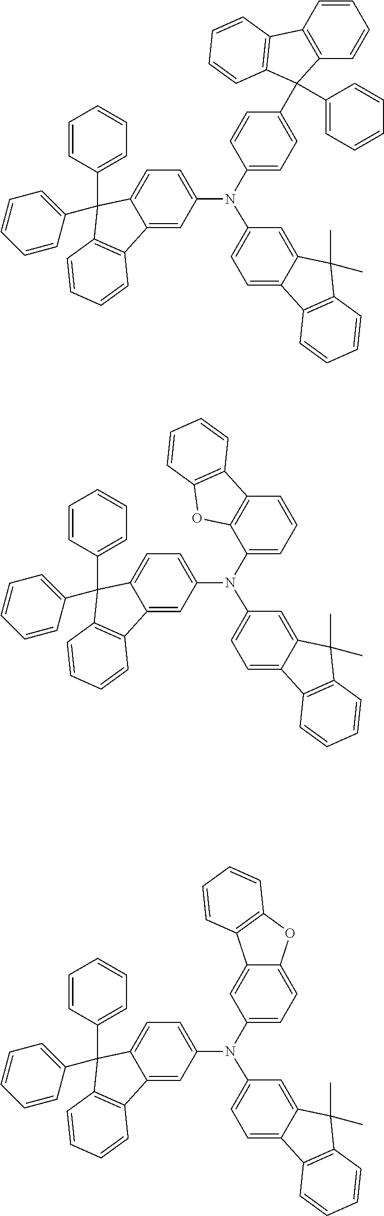 [SMILES: CC1(C)c2ccccc2-c2ccc(N(c3ccc(C4(c5ccccc5)c5ccccc5-c5ccccc54)cc3)c3ccc4c(c3)-c3ccccc3C4(c3ccccc3)c3ccccc3)cc21 ; CC1(C)c2ccccc2-c2ccc(N(c3ccc4c(c3)-c3ccccc3C4(c3ccccc3)c3ccccc3)c3cccc4c3oc3ccccc34)cc21 ; CC1(C)c2ccccc2-c2ccc(N(c3ccc4c(c3)-c3ccccc3C4(c3ccccc3)c3ccccc3)c3ccc4oc5ccccc5c4c3)cc21]

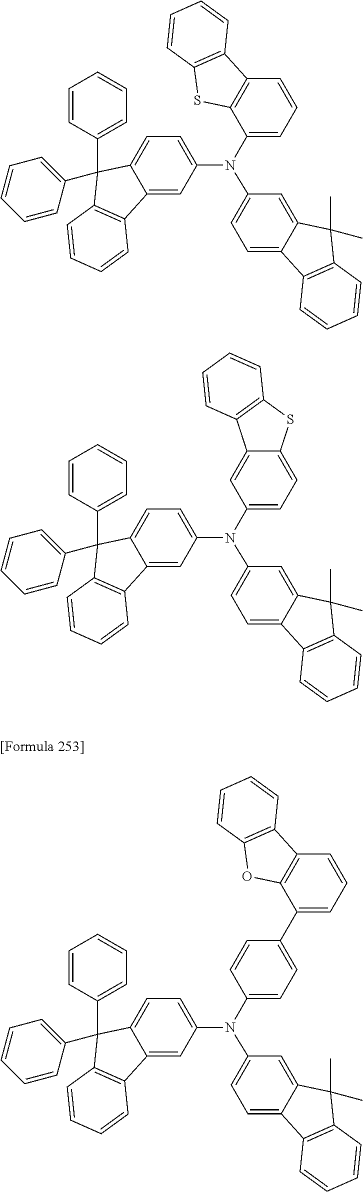 [SMILES: CC1(C)c2ccccc2-c2ccc(N(c3ccc4c(c3)-c3ccccc3C4(c3ccccc3)c3ccccc3)c3cccc4c3sc3ccccc34)cc21 ; CC1(C)c2ccccc2-c2ccc(N(c3ccc4c(c3)-c3ccccc3C4(c3ccccc3)c3ccccc3)c3ccc4sc5ccccc5c4c3)cc21 ; CC1(C)c2ccccc2-c2ccc(N(c3ccc(-c4cccc5c4oc4ccccc45)cc3)c3ccc4c(c3)-c3ccccc3C4(c3ccccc3)c3ccccc3)cc21]

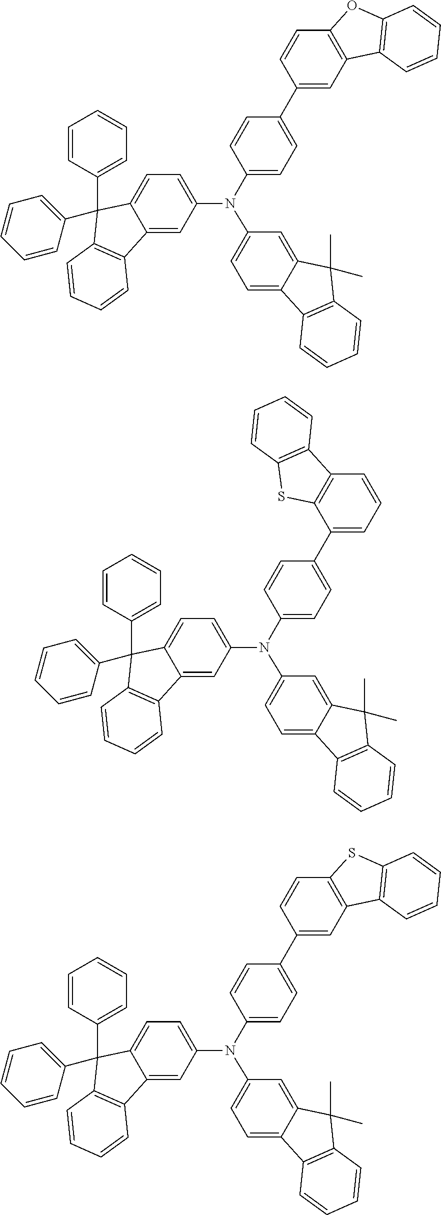

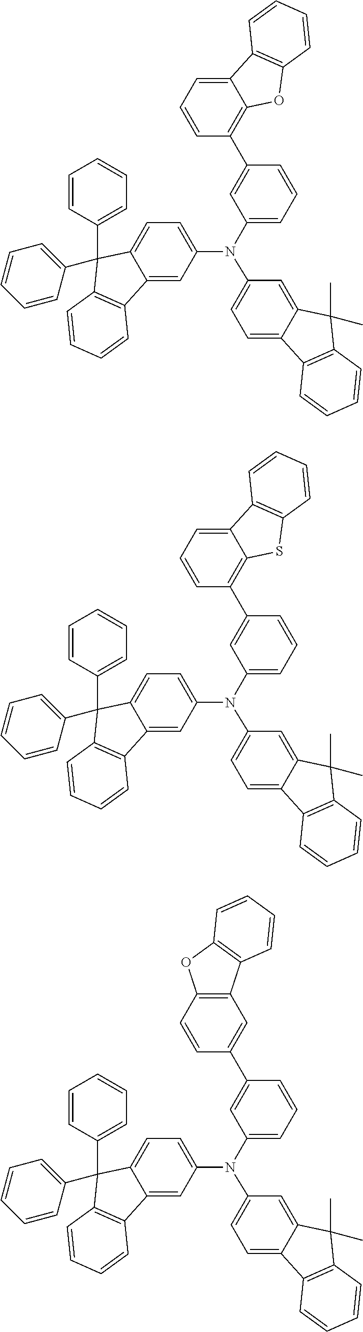 [SMILES: CC1(C)c2ccccc2-c2ccc(N(c3cccc(-c4cccc5c4oc4ccccc45)c3)c3ccc4c(c3)-c3ccccc3C4(c3ccccc3)c3ccccc3)cc21 ; CC1(C)c2ccccc2-c2ccc(N(c3cccc(-c4cccc5c4sc4ccccc45)c3)c3ccc4c(c3)-c3ccccc3C4(c3ccccc3)c3ccccc3)cc21 ; CC1(C)c2ccccc2-c2ccc(N(c3cccc(-c4ccc5oc6ccccc6c5c4)c3)c3ccc4c(c3)-c3ccccc3C4(c3ccccc3)c3ccccc3)cc21]

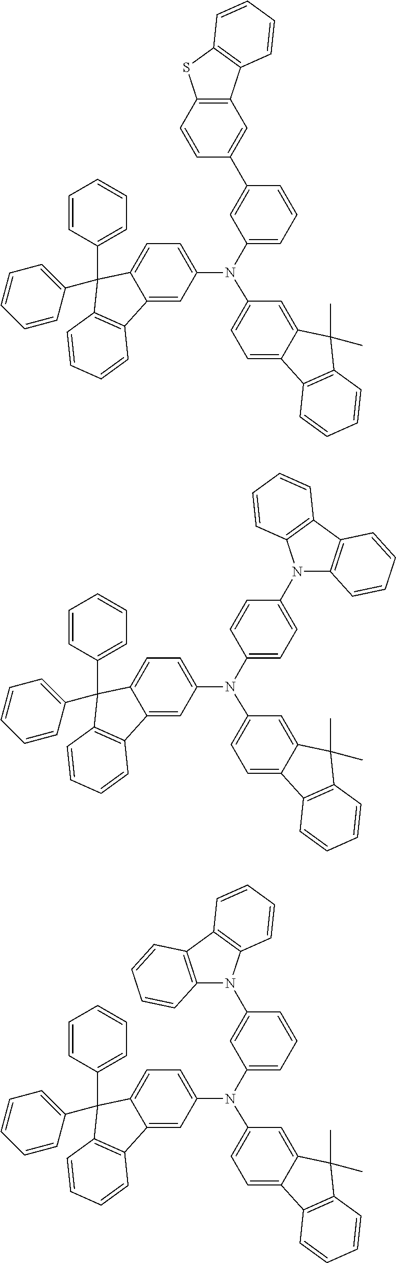 [SMILES: CC1(C)c2ccccc2-c2ccc(N(c3cccc(-c4ccc5sc6ccccc6c5c4)c3)c3ccc4c(c3)-c3ccccc3C4(c3ccccc3)c3ccccc3)cc21 ; CC1(C)c2ccccc2-c2ccc(N(c3ccc(-n4c5ccccc5c5ccccc54)cc3)c3ccc4c(c3)-c3ccccc3C4(c3ccccc3)c3ccccc3)cc21 ; CC1(C)c2ccccc2-c2ccc(N(c3cccc(-n4c5ccccc5c5ccccc54)c3)c3ccc4c(c3)-c3ccccc3C4(c3ccccc3)c3ccccc3)cc21]

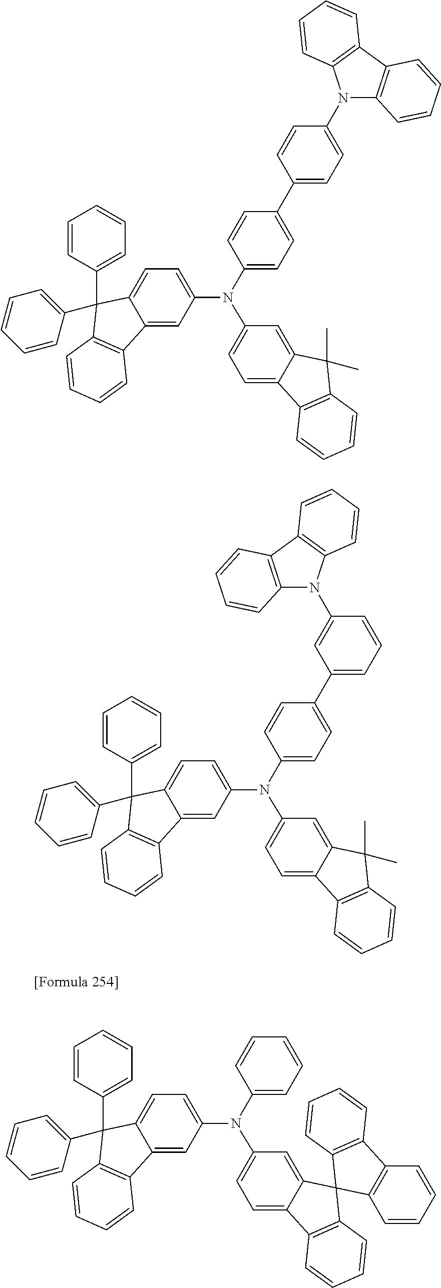

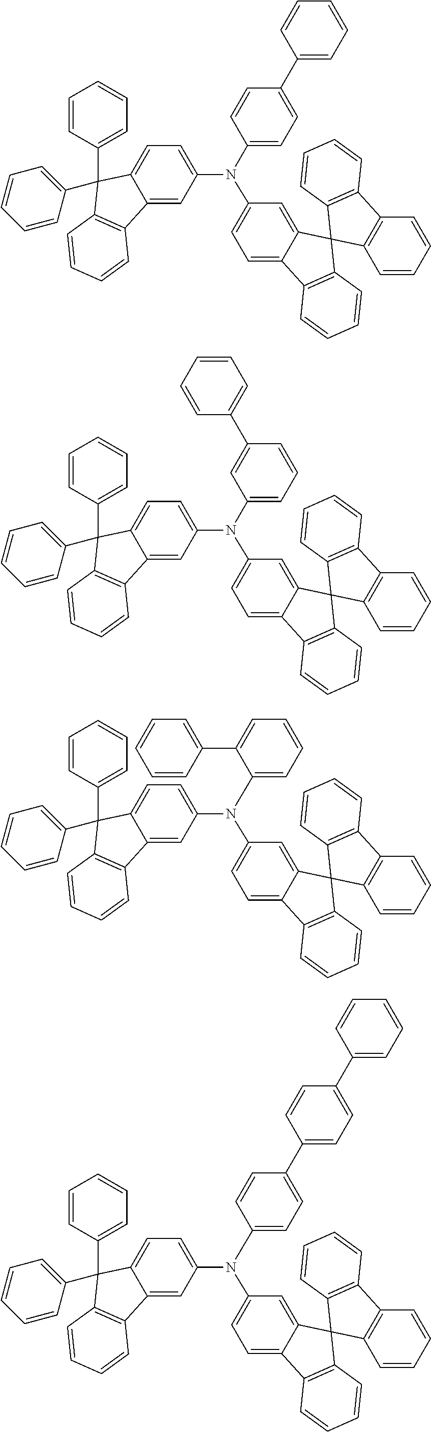

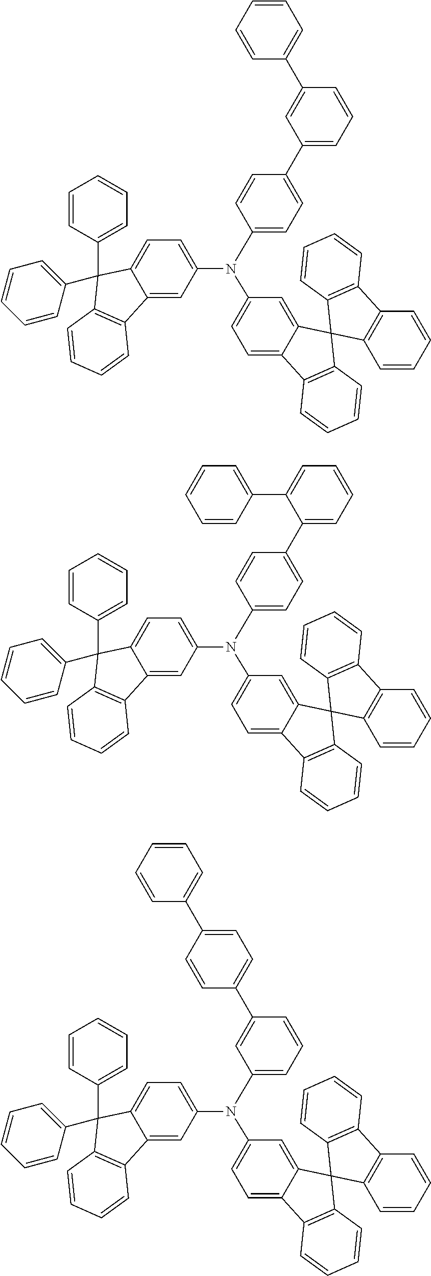

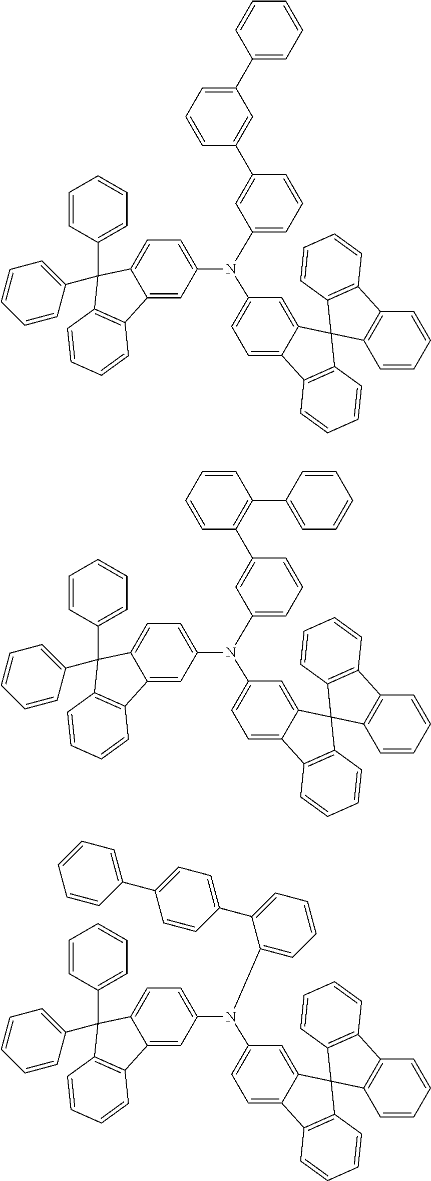

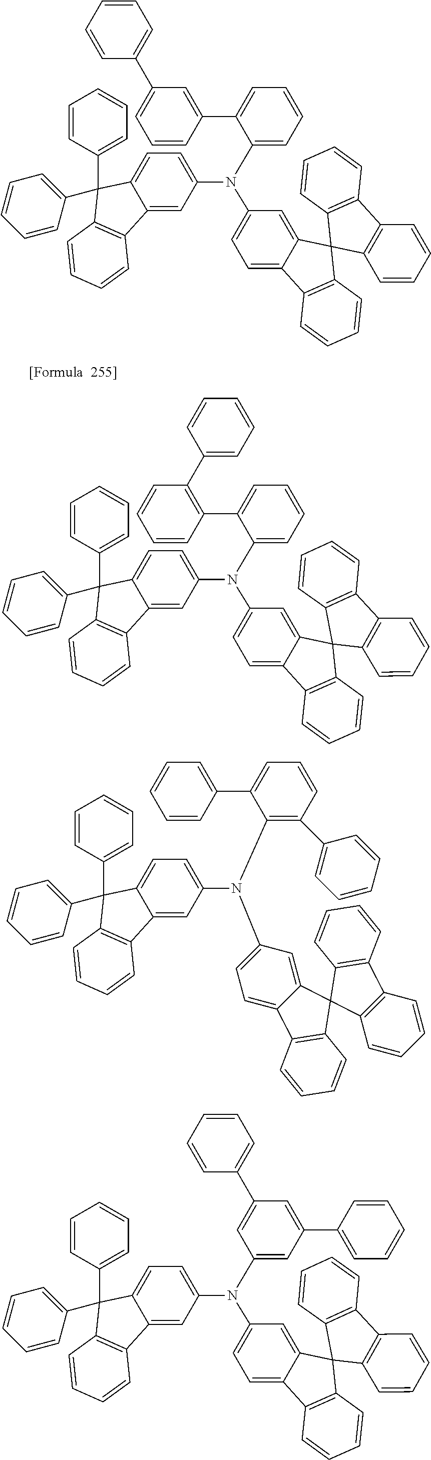

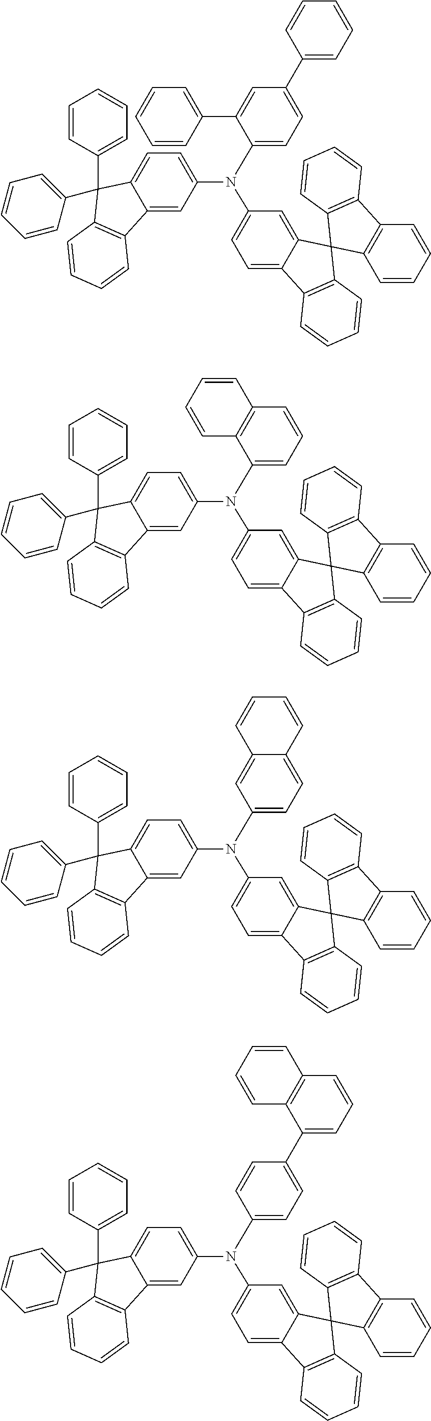 [SMILES: c1ccc(-c2ccc(N(c3ccc4c(c3)-c3ccccc3C4(c3ccccc3)c3ccccc3)c3ccc4c(c3)C3(c5ccccc5-c5ccccc53)c3ccccc3-4)c(-c3ccccc3)c2)cc1 ; c1ccc(C2(c3ccccc3)c3ccccc3-c3cc(N(c4ccc5c(c4)C4(c6ccccc6-c6ccccc64)c4ccccc4-5)c4cccc5ccccc45)ccc32)cc1 ; c1ccc(C2(c3ccccc3)c3ccccc3-c3cc(N(c4ccc5c(c4)C4(c6ccccc6-c6ccccc64)c4ccccc4-5)c4ccc5ccccc5c4)ccc32)cc1 ; c1ccc(C2(c3ccccc3)c3ccccc3-c3cc(N(c4ccc(-c5cccc6ccccc56)cc4)c4ccc5c(c4)C4(c6ccccc6-c6ccccc64)c4ccccc4-5)ccc32)cc1]

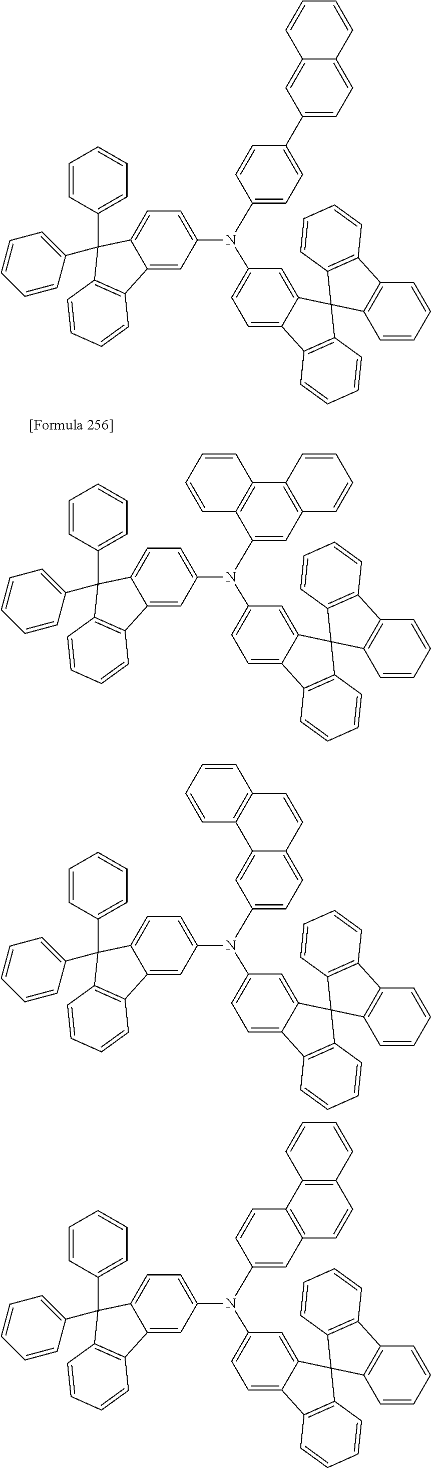

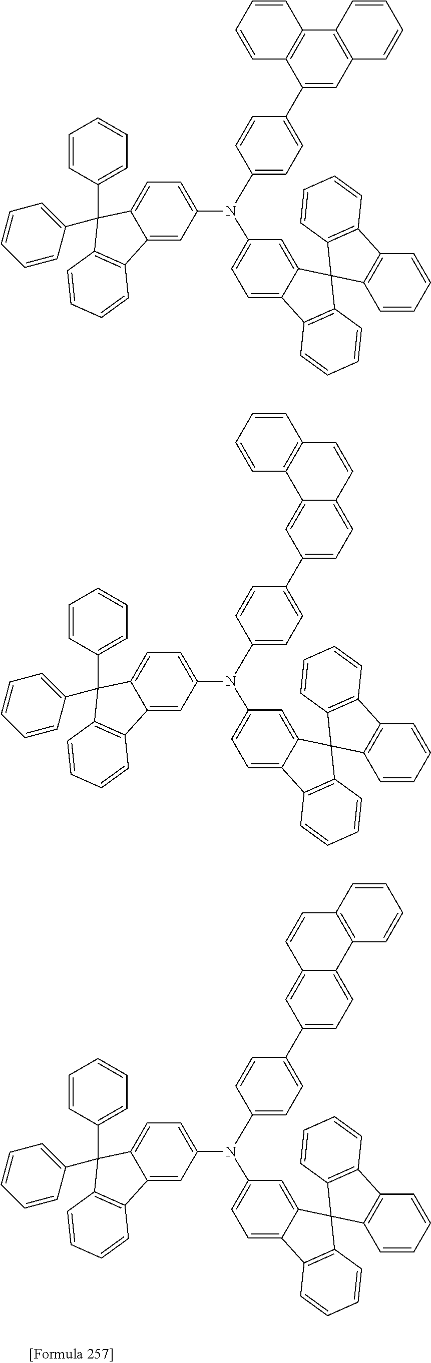

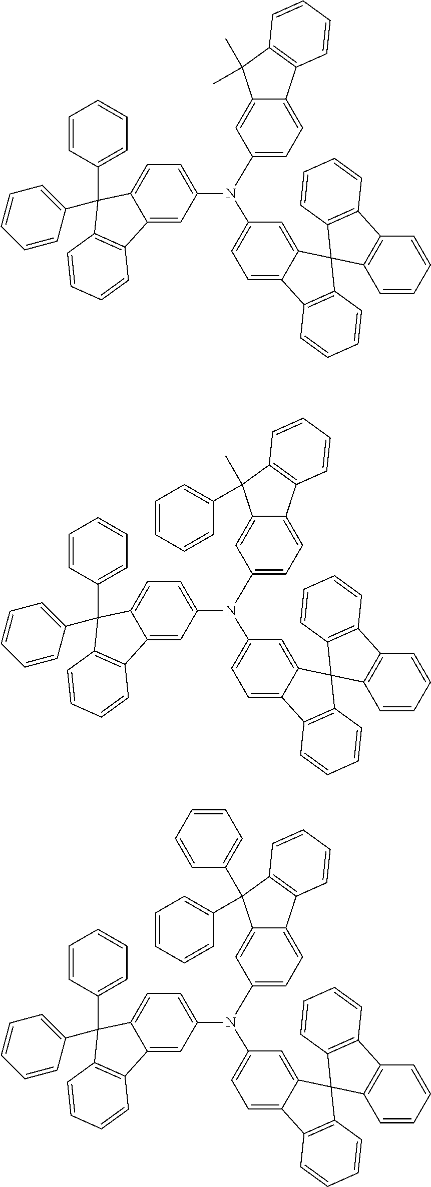 [SMILES: CC1(C)c2ccccc2-c2ccc(N(c3ccc4c(c3)-c3ccccc3C4(c3ccccc3)c3ccccc3)c3ccc4c(c3)C3(c5ccccc5-c5ccccc53)c3ccccc3-4)cc21 ; CC1(c2ccccc2)c2ccccc2-c2ccc(N(c3ccc4c(c3)-c3ccccc3C4(c3ccccc3)c3ccccc3)c3ccc4c(c3)C3(c5ccccc5-c5ccccc53)c3ccccc3-4)cc21 ; c1ccc(C2(c3ccccc3)c3ccccc3-c3cc(N(c4ccc5c(c4)C(c4ccccc4)(c4ccccc4)c4ccccc4-5)c4ccc5c(c4)C4(c6ccccc6-c6ccccc64)c4ccccc4-5)ccc32)cc1]

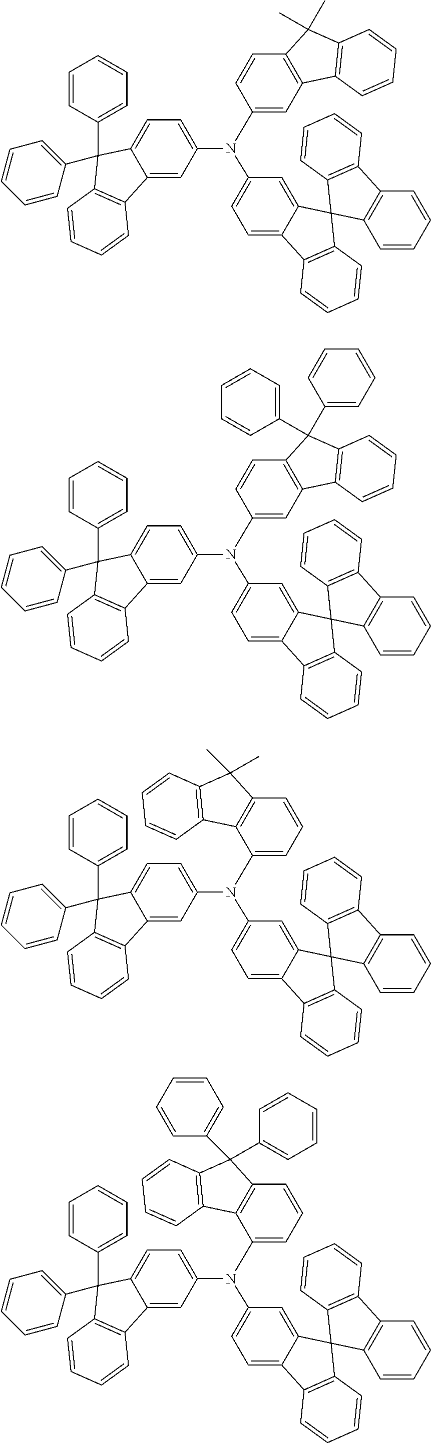

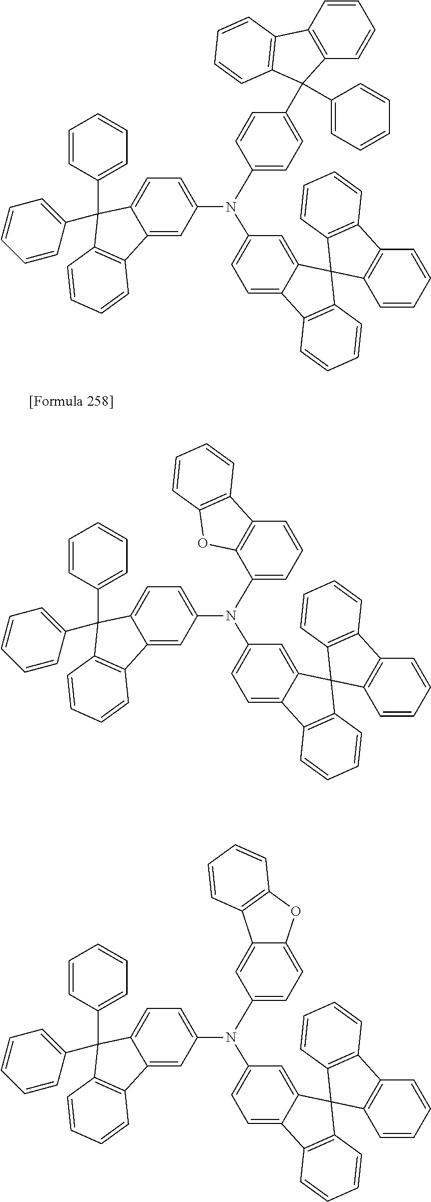 [SMILES: c1ccc(C2(c3ccc(N(c4ccc5c(c4)-c4ccccc4C5(c4ccccc4)c4ccccc4)c4ccc5c(c4)C4(c6ccccc6-c6ccccc64)c4ccccc4-5)cc3)c3ccccc3-c3ccccc32)cc1 ; c1ccc(C2(c3ccccc3)c3ccccc3-c3cc(N(c4ccc5c(c4)C4(c6ccccc6-c6ccccc64)c4ccccc4-5)c4cccc5c4oc4ccccc45)ccc32)cc1 ; c1ccc(C2(c3ccccc3)c3ccccc3-c3cc(N(c4ccc5c(c4)C4(c6ccccc6-c6ccccc64)c4ccccc4-5)c4ccc5oc6ccccc6c5c4)ccc32)cc1]

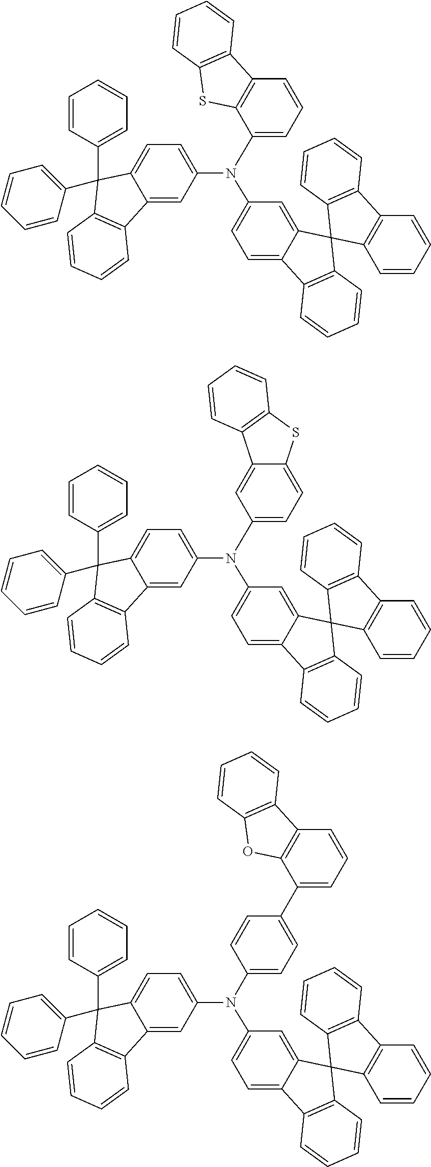 [SMILES: c1ccc(C2(c3ccccc3)c3ccccc3-c3cc(N(c4ccc5c(c4)C4(c6ccccc6-c6ccccc64)c4ccccc4-5)c4cccc5c4sc4ccccc45)ccc32)cc1 ; c1ccc(C2(c3ccccc3)c3ccccc3-c3cc(N(c4ccc5c(c4)C4(c6ccccc6-c6ccccc64)c4ccccc4-5)c4ccc5sc6ccccc6c5c4)ccc32)cc1 ; c1ccc(C2(c3ccccc3)c3ccccc3-c3cc(N(c4ccc(-c5cccc6c5oc5ccccc56)cc4)c4ccc5c(c4)C4(c6ccccc6-c6ccccc64)c4ccccc4-5)ccc32)cc1]

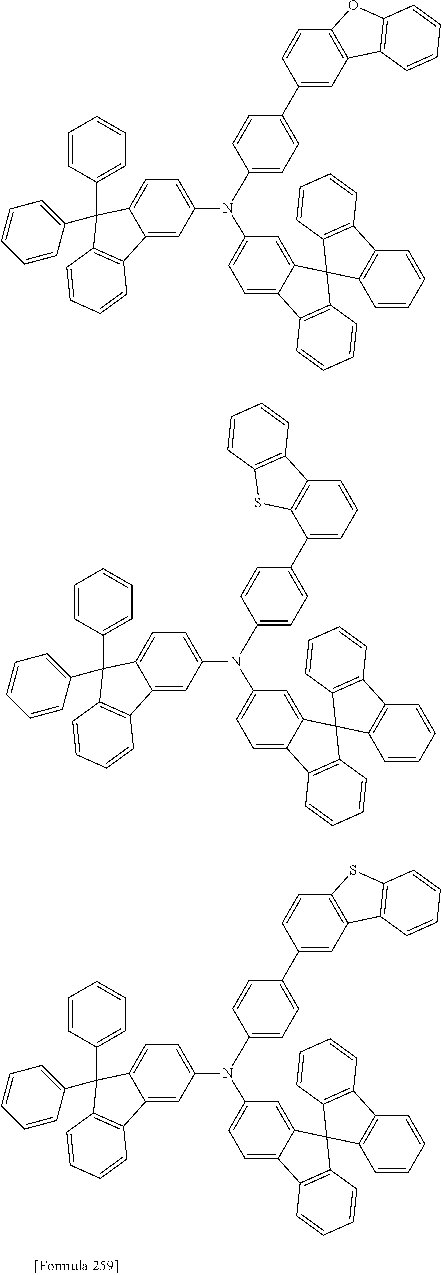 [SMILES: c1ccc(C2(c3ccccc3)c3ccccc3-c3cc(N(c4ccc(-c5ccc6oc7ccccc7c6c5)cc4)c4ccc5c(c4)C4(c6ccccc6-c6ccccc64)c4ccccc4-5)ccc32)cc1 ; c1ccc(C2(c3ccccc3)c3ccccc3-c3cc(N(c4ccc(-c5cccc6c5sc5ccccc56)cc4)c4ccc5c(c4)C4(c6ccccc6-c6ccccc64)c4ccccc4-5)ccc32)cc1 ; c1ccc(C2(c3ccccc3)c3ccccc3-c3cc(N(c4ccc(-c5ccc6sc7ccccc7c6c5)cc4)c4ccc5c(c4)C4(c6ccccc6-c6ccccc64)c4ccccc4-5)ccc32)cc1]

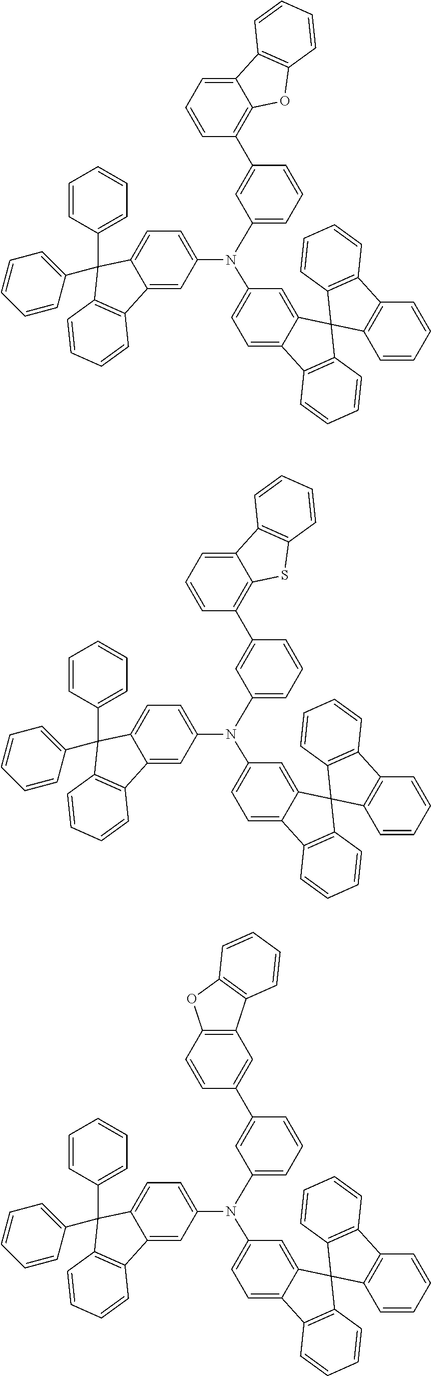 [SMILES: c1ccc(C2(c3ccccc3)c3ccccc3-c3cc(N(c4cccc(-c5cccc6c5oc5ccccc56)c4)c4ccc5c(c4)C4(c6ccccc6-c6ccccc64)c4ccccc4-5)ccc32)cc1 ; c1ccc(C2(c3ccccc3)c3ccccc3-c3cc(N(c4cccc(-c5cccc6c5sc5ccccc56)c4)c4ccc5c(c4)C4(c6ccccc6-c6ccccc64)c4ccccc4-5)ccc32)cc1 ; c1ccc(C2(c3ccccc3)c3ccccc3-c3cc(N(c4cccc(-c5ccc6oc7ccccc7c6c5)c4)c4ccc5c(c4)C4(c6ccccc6-c6ccccc64)c4ccccc4-5)ccc32)cc1]

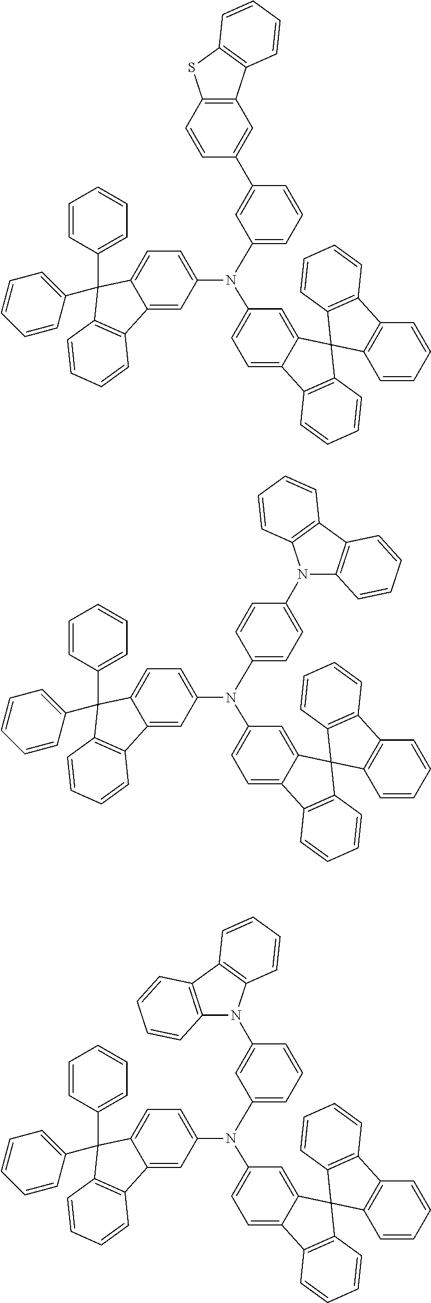 [SMILES: c1ccc(C2(c3ccccc3)c3ccccc3-c3cc(N(c4cccc(-c5ccc6sc7ccccc7c6c5)c4)c4ccc5c(c4)C4(c6ccccc6-c6ccccc64)c4ccccc4-5)ccc32)cc1 ; c1ccc(C2(c3ccccc3)c3ccccc3-c3cc(N(c4ccc(-n5c6ccccc6c6ccccc65)cc4)c4ccc5c(c4)C4(c6ccccc6-c6ccccc64)c4ccccc4-5)ccc32)cc1 ; c1ccc(C2(c3ccccc3)c3ccccc3-c3cc(N(c4cccc(-n5c6ccccc6c6ccccc65)c4)c4ccc5c(c4)C4(c6ccccc6-c6ccccc64)c4ccccc4-5)ccc32)cc1]

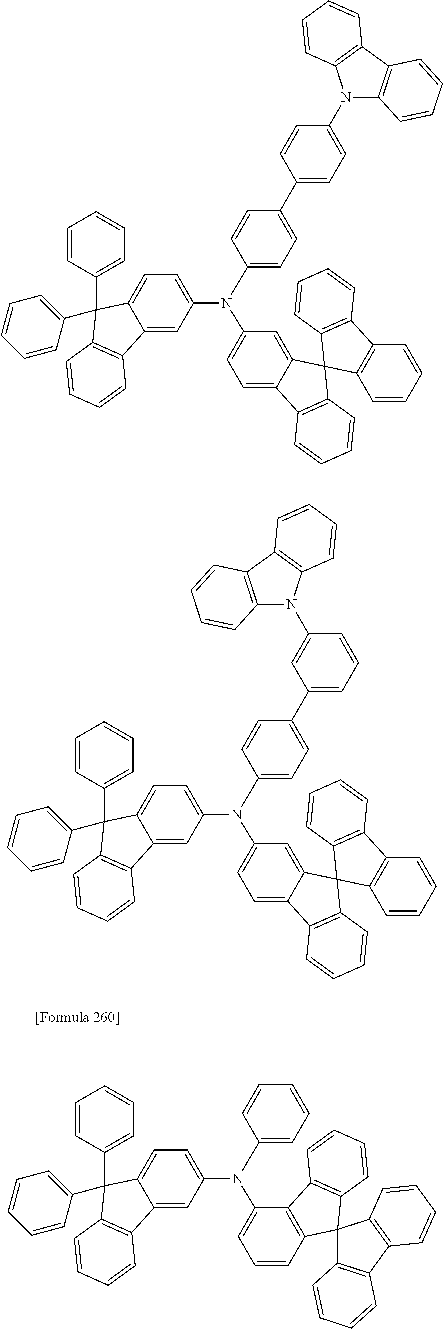

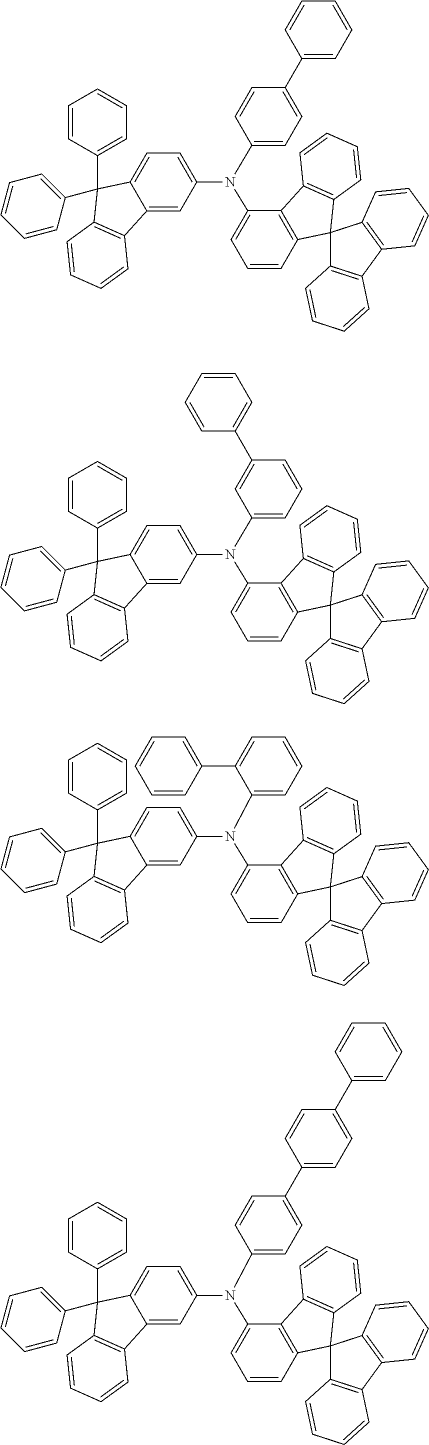

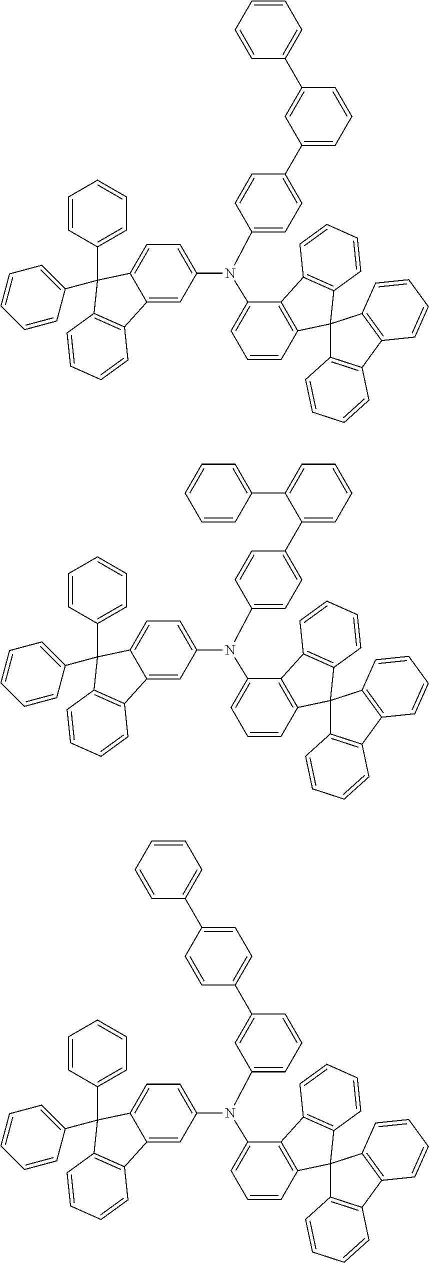

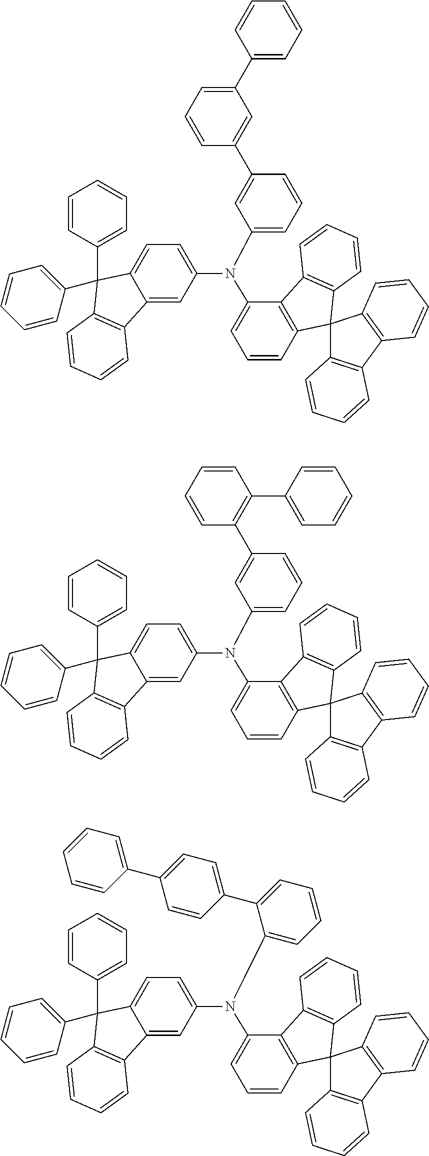

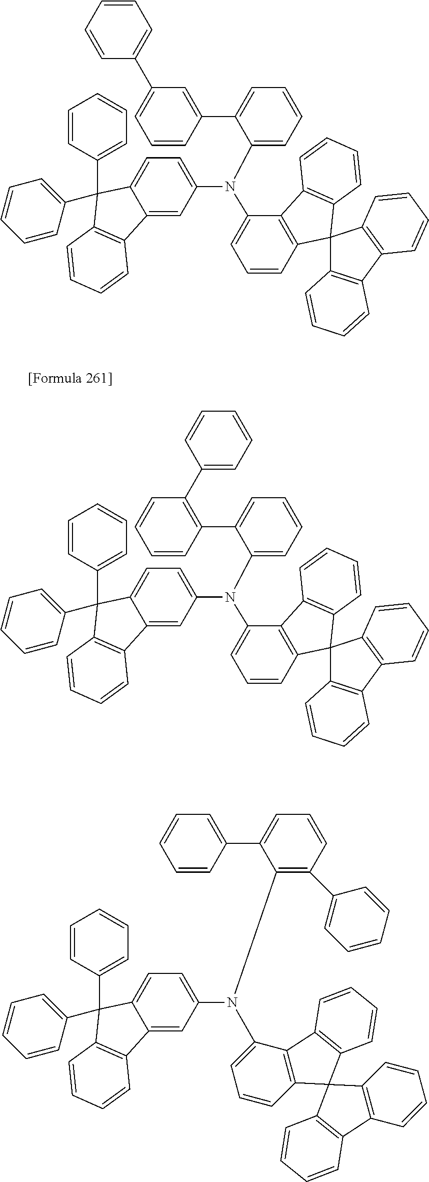 [SMILES: c1ccc(-c2cccc(-c3ccccc3N(c3ccc4c(c3)-c3ccccc3C4(c3ccccc3)c3ccccc3)c3cccc4c3-c3ccccc3C43c4ccccc4-c4ccccc43)c2)cc1 ; c1ccc(-c2ccccc2-c2ccccc2N(c2ccc3c(c2)-c2ccccc2C3(c2ccccc2)c2ccccc2)c2cccc3c2-c2ccccc2C32c3ccccc3-c3ccccc32)cc1 ; c1ccc(-c2cccc(-c3ccccc3)c2N(c2ccc3c(c2)-c2ccccc2C3(c2ccccc2)c2ccccc2)c2cccc3c2-c2ccccc2C32c3ccccc3-c3ccccc32)cc1]

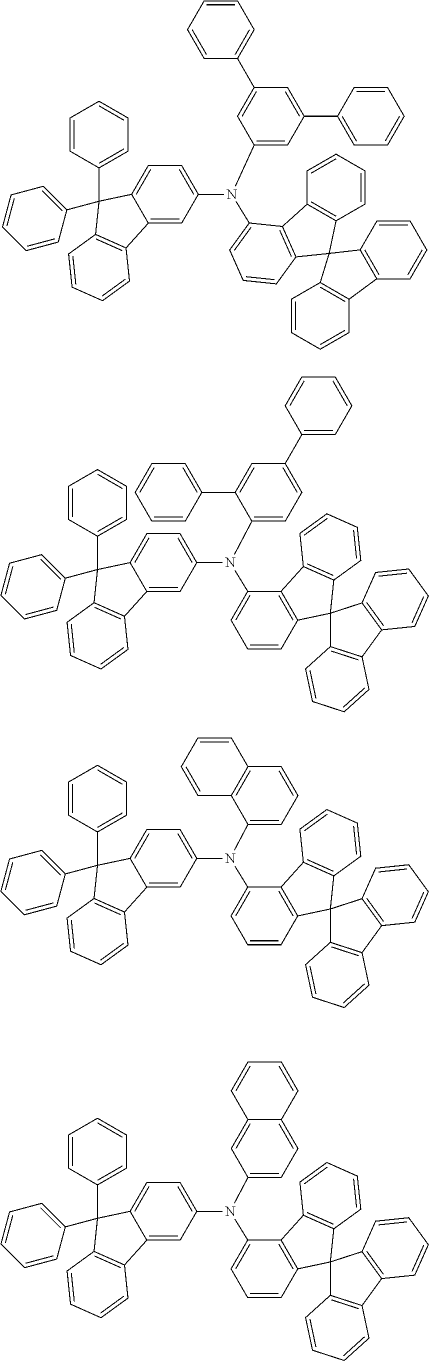

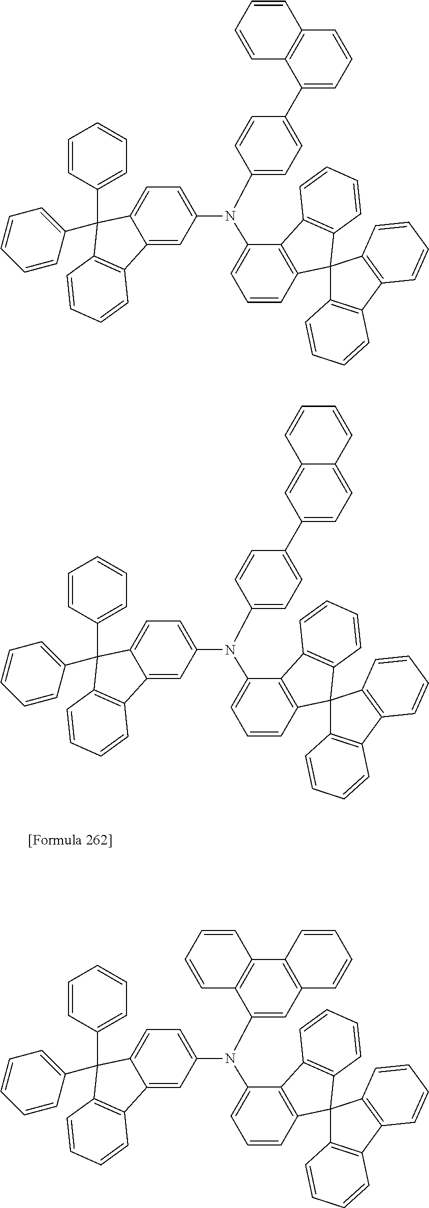

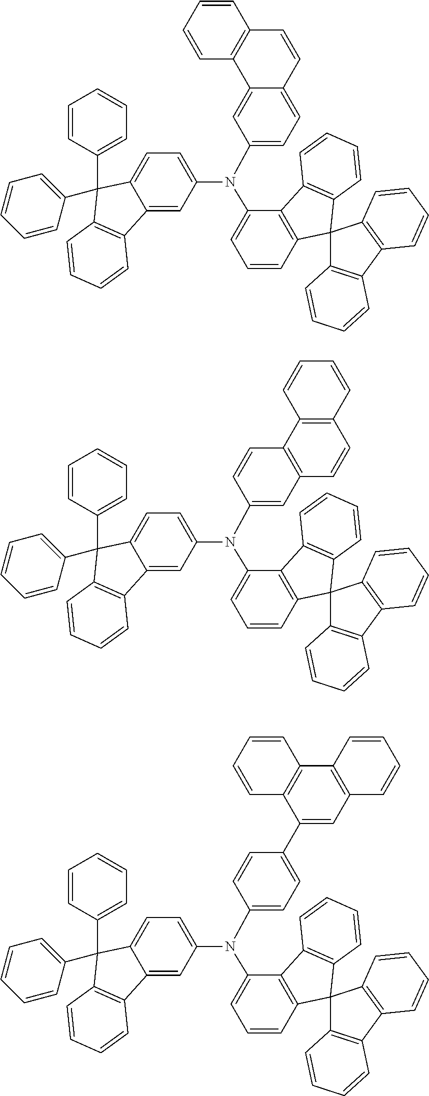

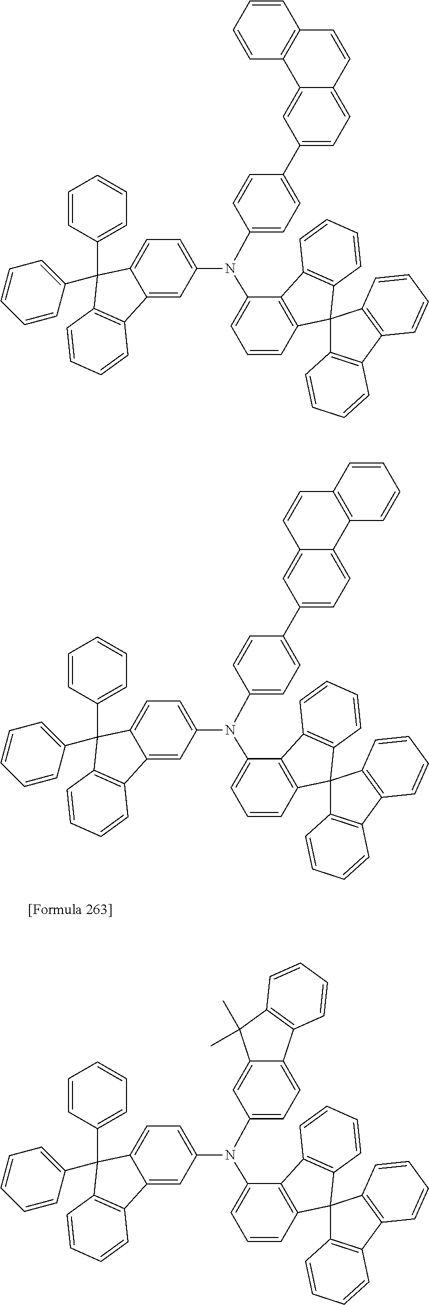

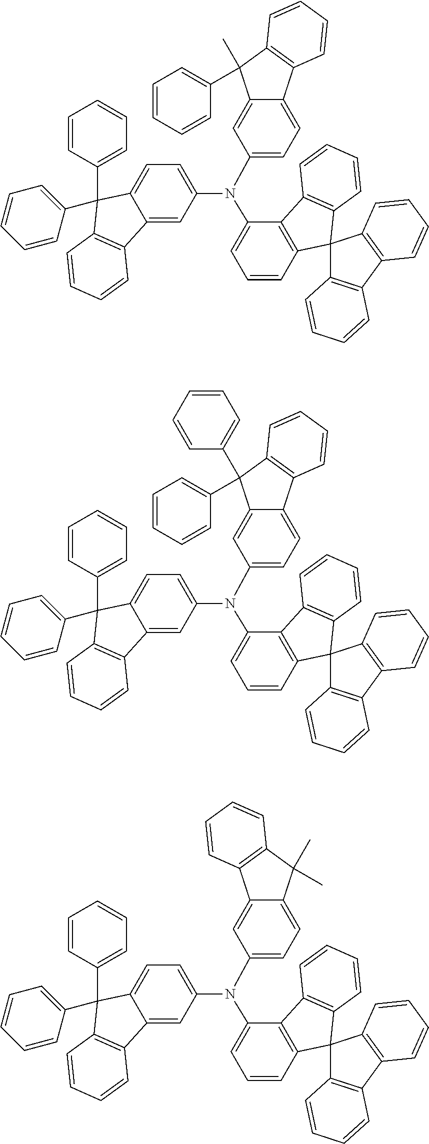 [SMILES: CC1(c2ccccc2)c2ccccc2-c2ccc(N(c3ccc4c(c3)-c3ccccc3C4(c3ccccc3)c3ccccc3)c3cccc4c3-c3ccccc3C43c4ccccc4-c4ccccc43)cc21 ; c1ccc(C2(c3ccccc3)c3ccccc3-c3cc(N(c4ccc5c(c4)C(c4ccccc4)(c4ccccc4)c4ccccc4-5)c4cccc5c4-c4ccccc4C54c5ccccc5-c5ccccc54)ccc32)cc1 ; CC1(C)c2ccccc2-c2cc(N(c3ccc4c(c3)-c3ccccc3C4(c3ccccc3)c3ccccc3)c3cccc4c3-c3ccccc3C43c4ccccc4-c4ccccc43)ccc21]

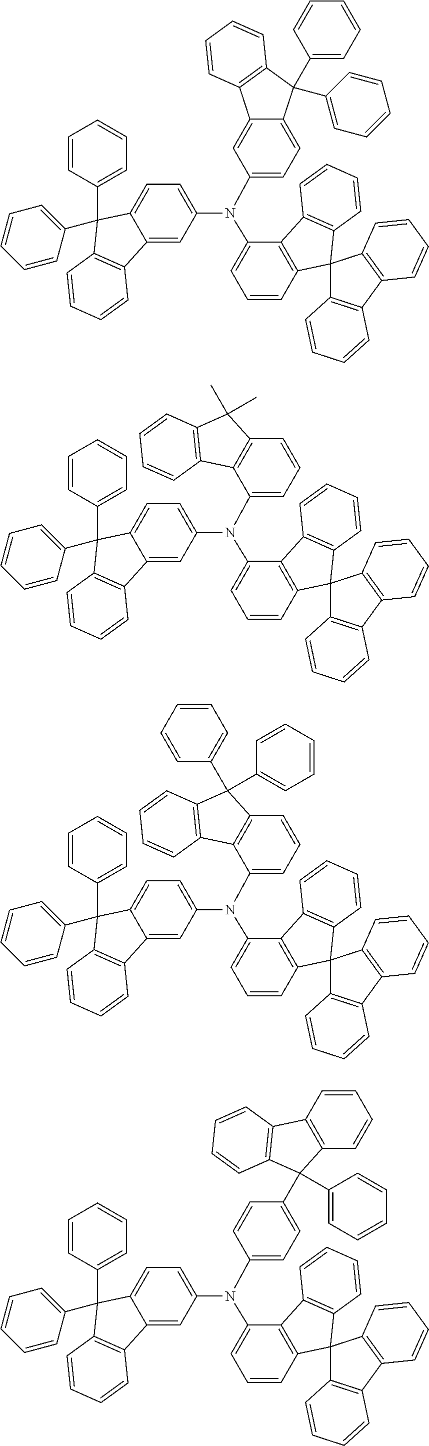

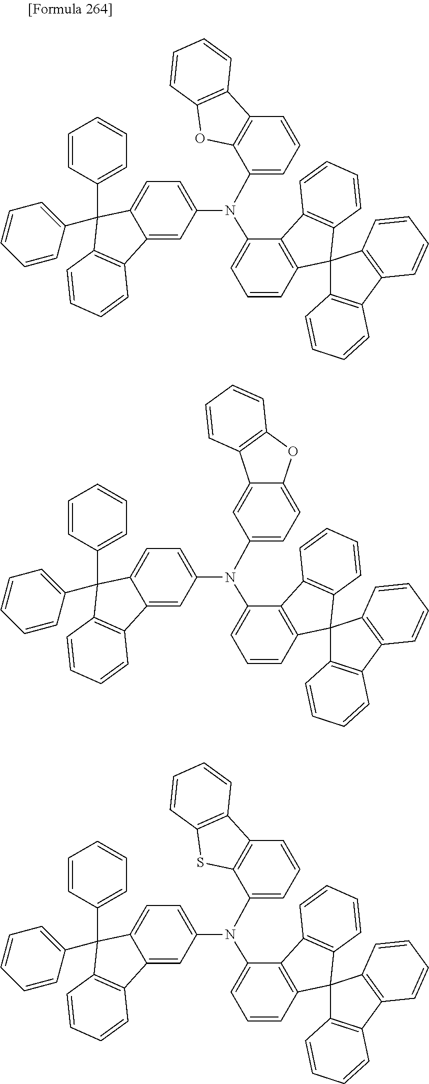 [SMILES: c1ccc(C2(c3ccccc3)c3ccccc3-c3cc(N(c4cccc5c4-c4ccccc4C54c5ccccc5-c5ccccc54)c4cccc5c4oc4ccccc45)ccc32)cc1 ; c1ccc(C2(c3ccccc3)c3ccccc3-c3cc(N(c4ccc5oc6ccccc6c5c4)c4cccc5c4-c4ccccc4C54c5ccccc5-c5ccccc54)ccc32)cc1 ; c1ccc(C2(c3ccccc3)c3ccccc3-c3cc(N(c4cccc5c4-c4ccccc4C54c5ccccc5-c5ccccc54)c4cccc5c4sc4ccccc45)ccc32)cc1]

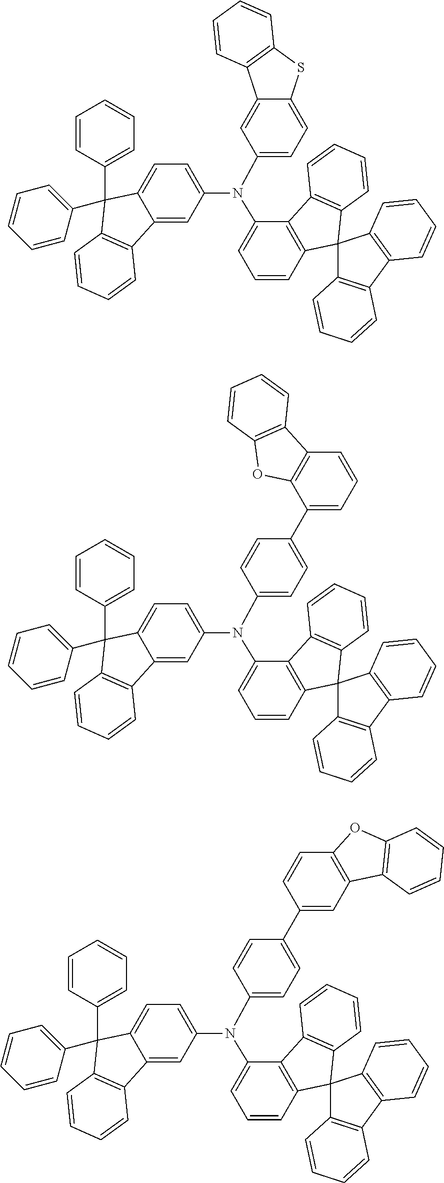

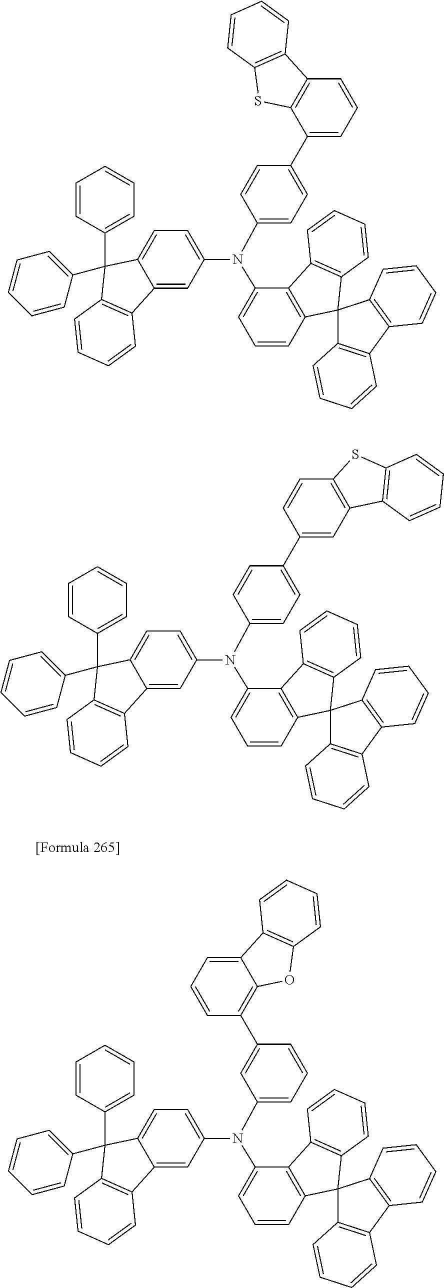

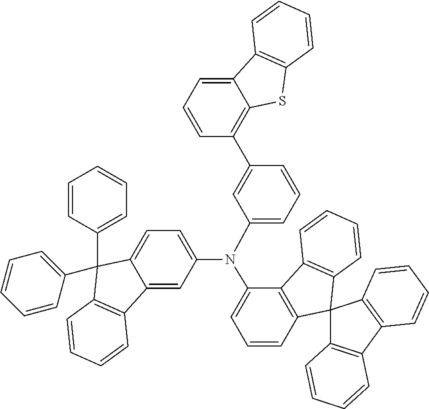 [SMILES: c1ccc(C2(c3ccccc3)c3ccccc3-c3cc(N(c4cccc(-c5cccc6c5sc5ccccc56)c4)c4cccc5c4-c4ccccc4C54c5ccccc5-c5ccccc54)ccc32)cc1]

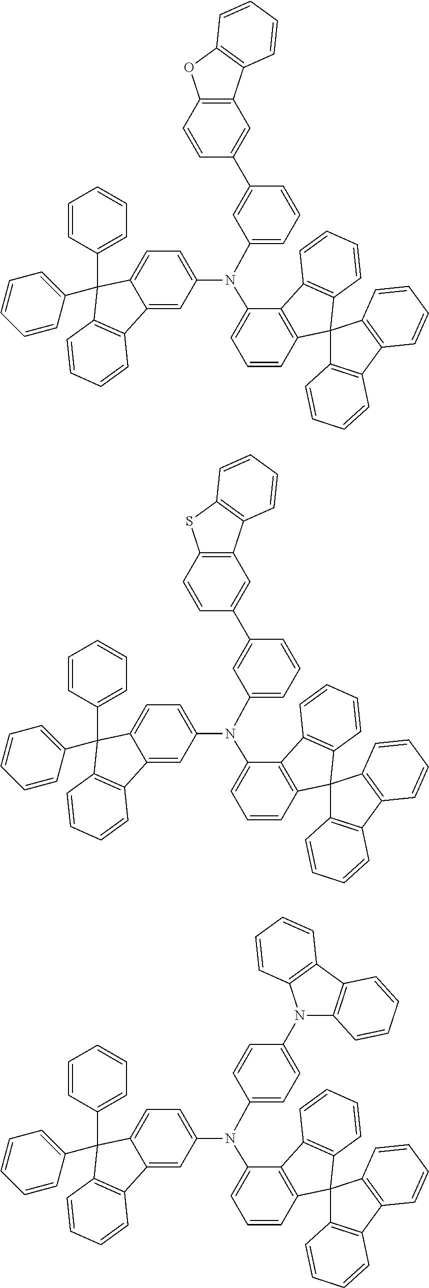 [SMILES: c1ccc(C2(c3ccccc3)c3ccccc3-c3cc(N(c4cccc(-c5ccc6oc7ccccc7c6c5)c4)c4cccc5c4-c4ccccc4C54c5ccccc5-c5ccccc54)ccc32)cc1 ; c1ccc(C2(c3ccccc3)c3ccccc3-c3cc(N(c4cccc(-c5ccc6sc7ccccc7c6c5)c4)c4cccc5c4-c4ccccc4C54c5ccccc5-c5ccccc54)ccc32)cc1 ; c1ccc(C2(c3ccccc3)c3ccccc3-c3cc(N(c4ccc(-n5c6ccccc6c6ccccc65)cc4)c4cccc5c4-c4ccccc4C54c5ccccc5-c5ccccc54)ccc32)cc1]

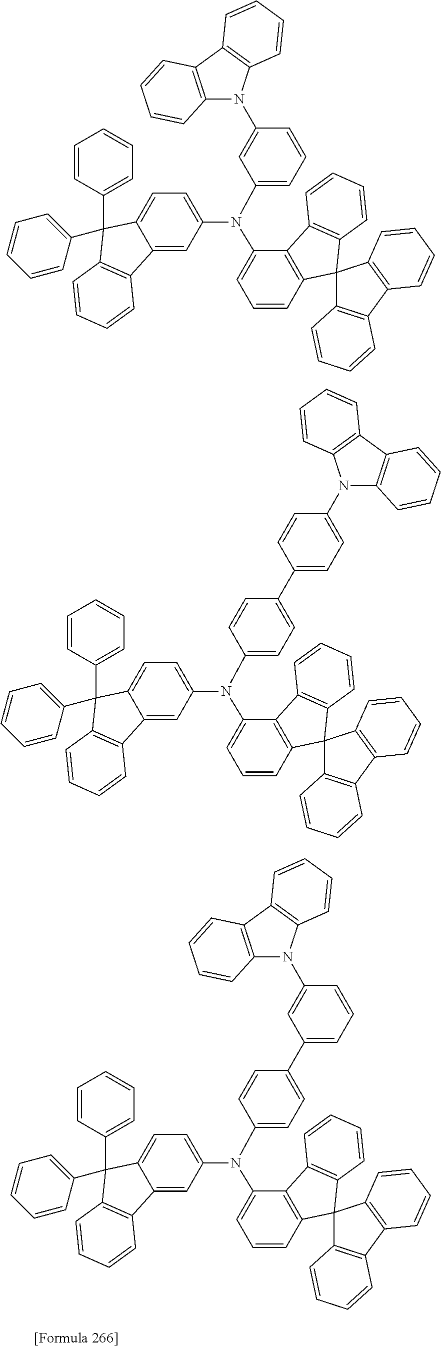

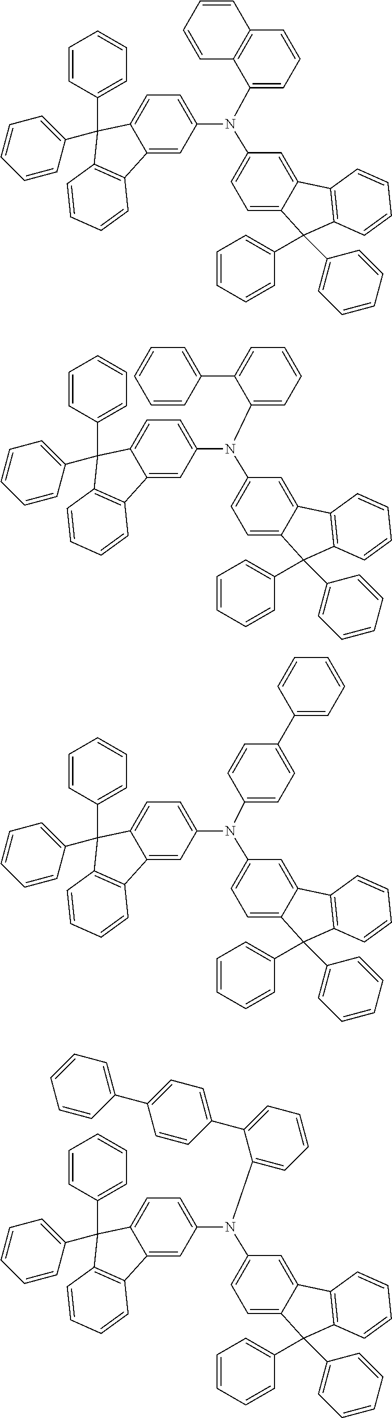

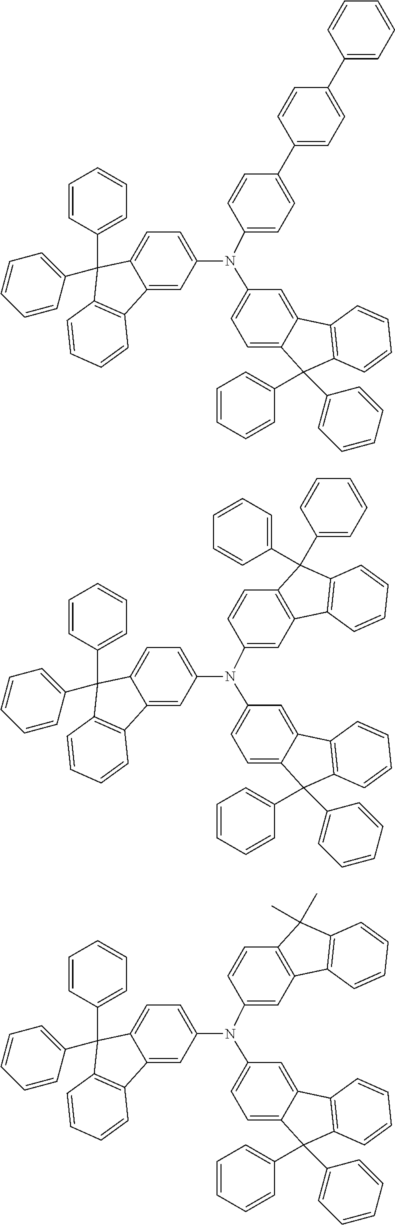

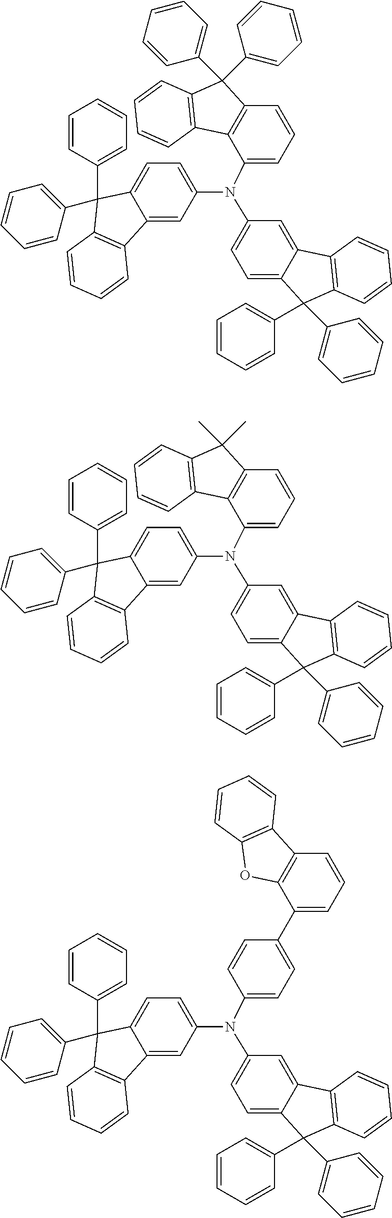

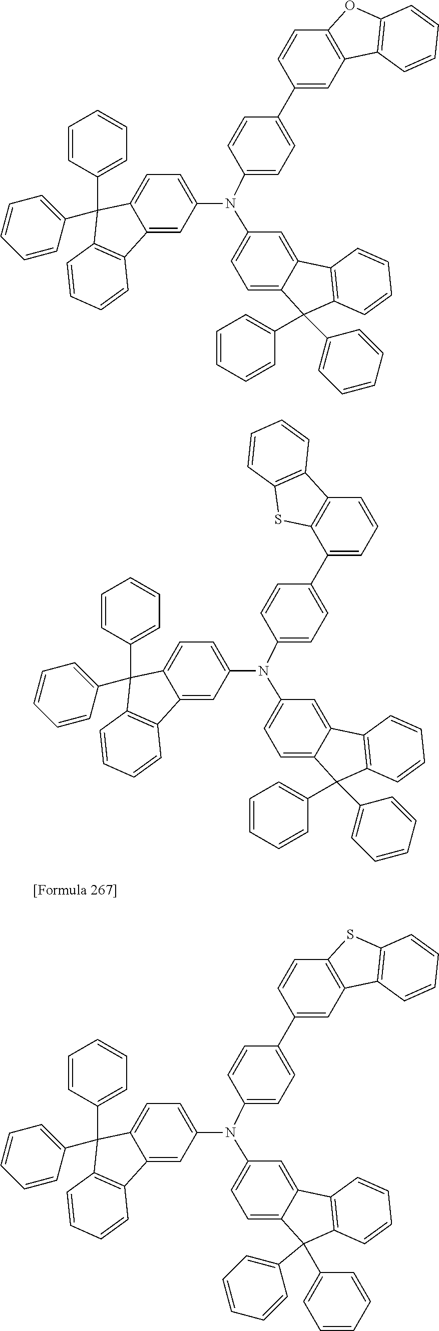 [SMILES: c1ccc(C2(c3ccccc3)c3ccccc3-c3cc(N(c4ccc(-c5ccc6oc7ccccc7c6c5)cc4)c4ccc5c(c4)-c4ccccc4C5(c4ccccc4)c4ccccc4)ccc32)cc1 ; c1ccc(C2(c3ccccc3)c3ccccc3-c3cc(N(c4ccc(-c5cccc6c5sc5ccccc56)cc4)c4ccc5c(c4)-c4ccccc4C5(c4ccccc4)c4ccccc4)ccc32)cc1 ; c1ccc(C2(c3ccccc3)c3ccccc3-c3cc(N(c4ccc(-c5ccc6sc7ccccc7c6c5)cc4)c4ccc5c(c4)-c4ccccc4C5(c4ccccc4)c4ccccc4)ccc32)cc1]

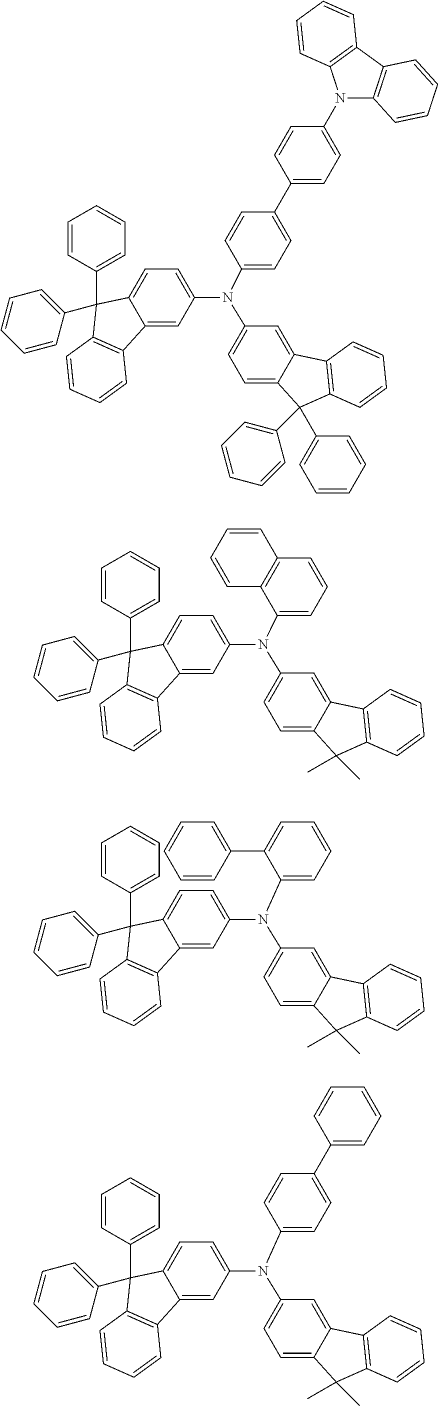

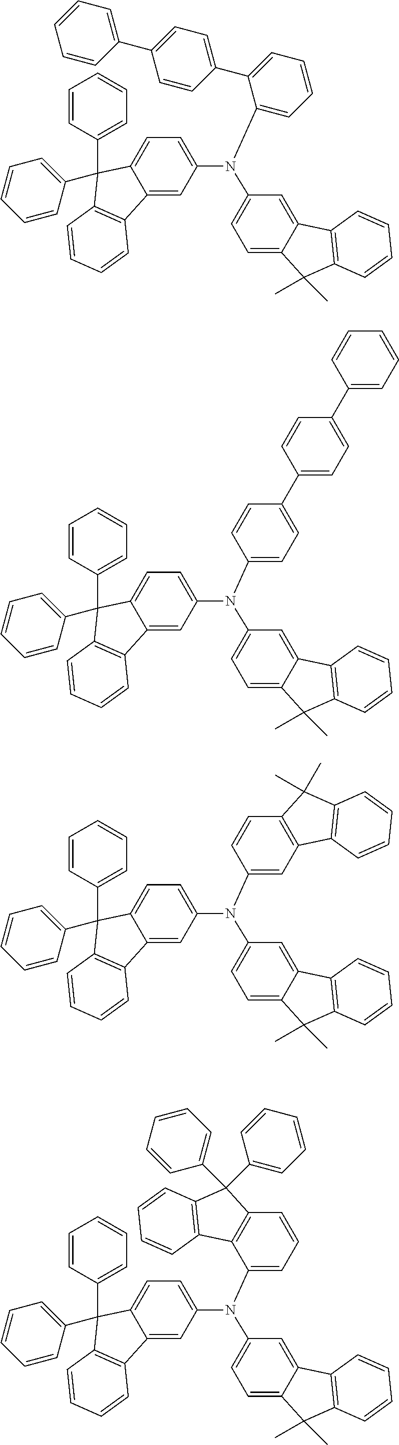

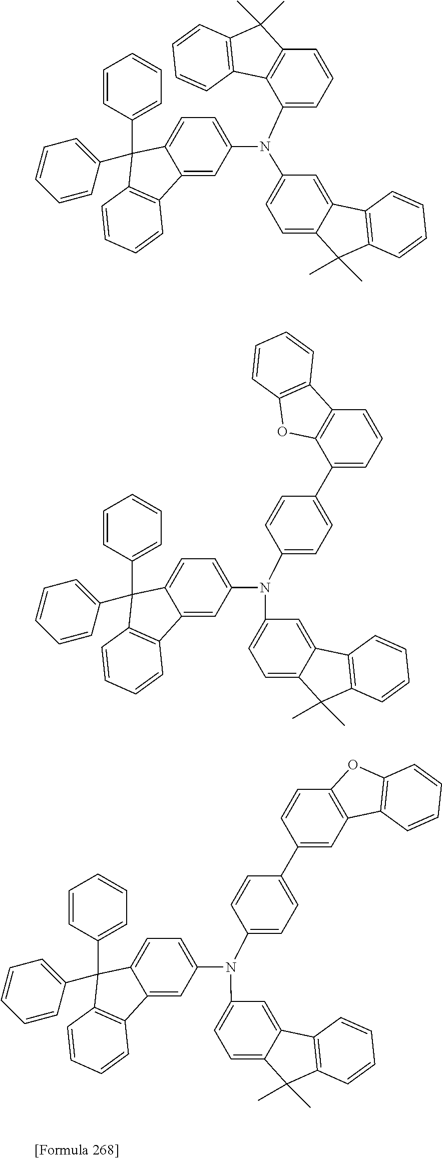

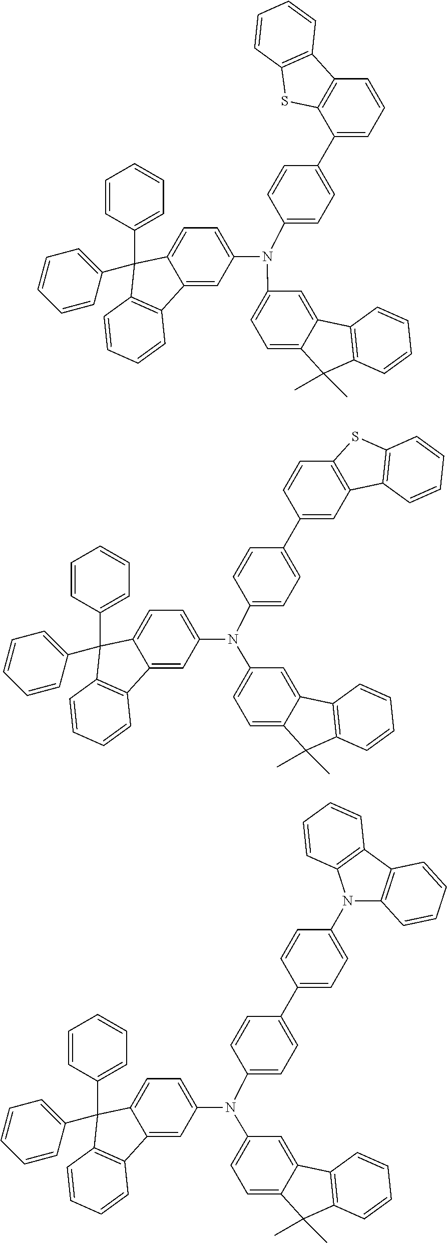

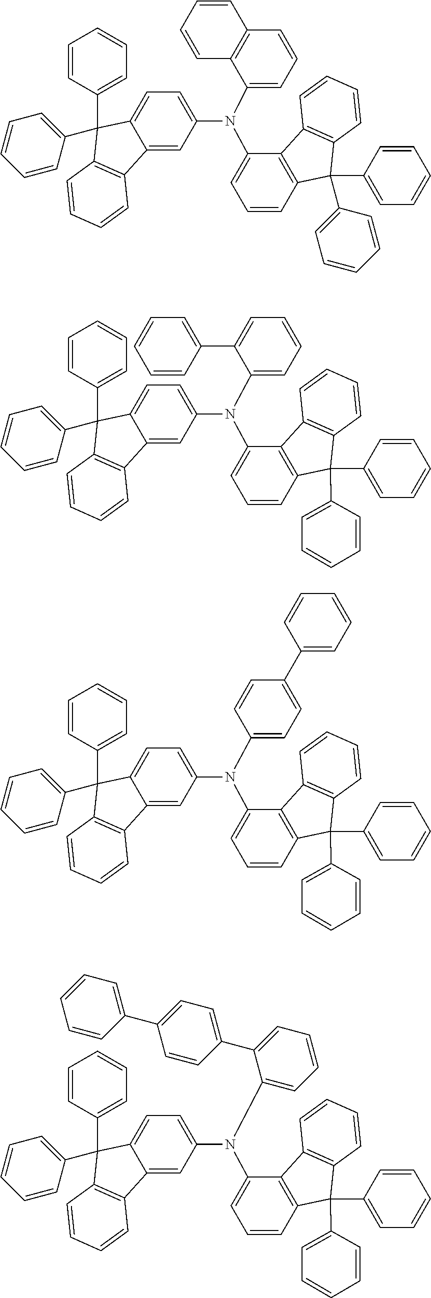

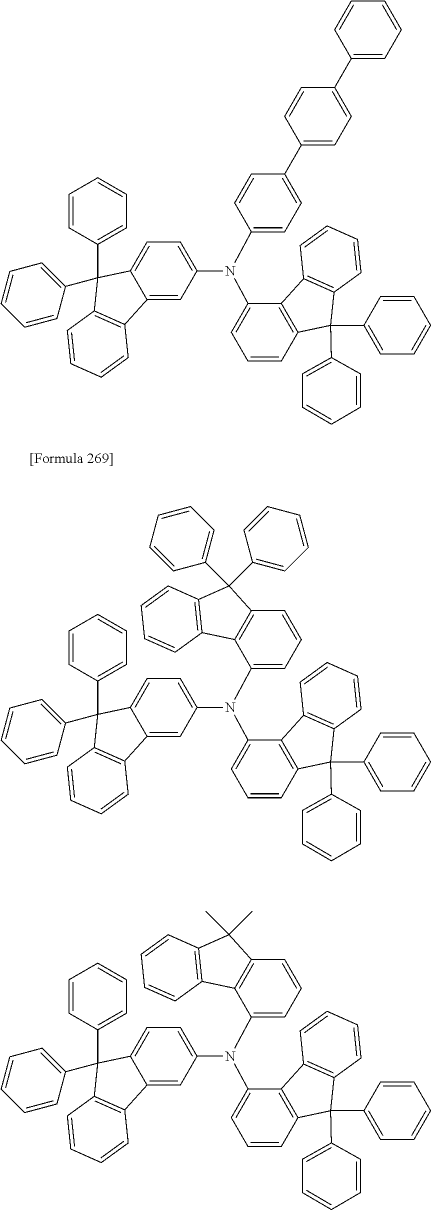 [SMILES: c1ccc(-c2ccc(-c3ccc(N(c4ccc5c(c4)-c4ccccc4C5(c4ccccc4)c4ccccc4)c4cccc5c4-c4ccccc4C5(c4ccccc4)c4ccccc4)cc3)cc2)cc1 ; c1ccc(C2(c3ccccc3)c3ccccc3-c3cc(N(c4cccc5c4-c4ccccc4C5(c4ccccc4)c4ccccc4)c4cccc5c4-c4ccccc4C5(c4ccccc4)c4ccccc4)ccc32)cc1 ; CC1(C)c2ccccc2-c2c(N(c3ccc4c(c3)-c3ccccc3C4(c3ccccc3)c3ccccc3)c3cccc4c3-c3ccccc3C4(c3ccccc3)c3ccccc3)cccc21]

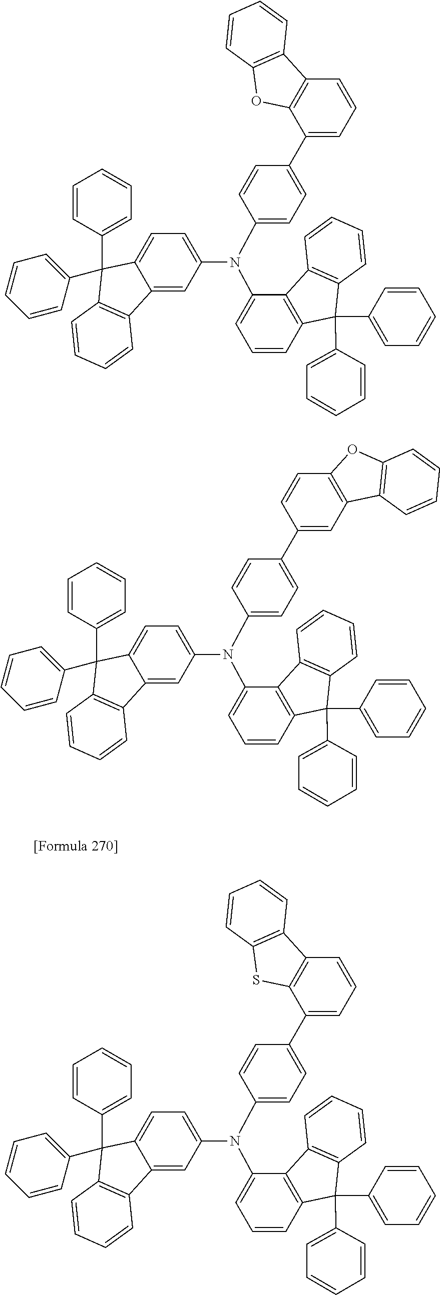

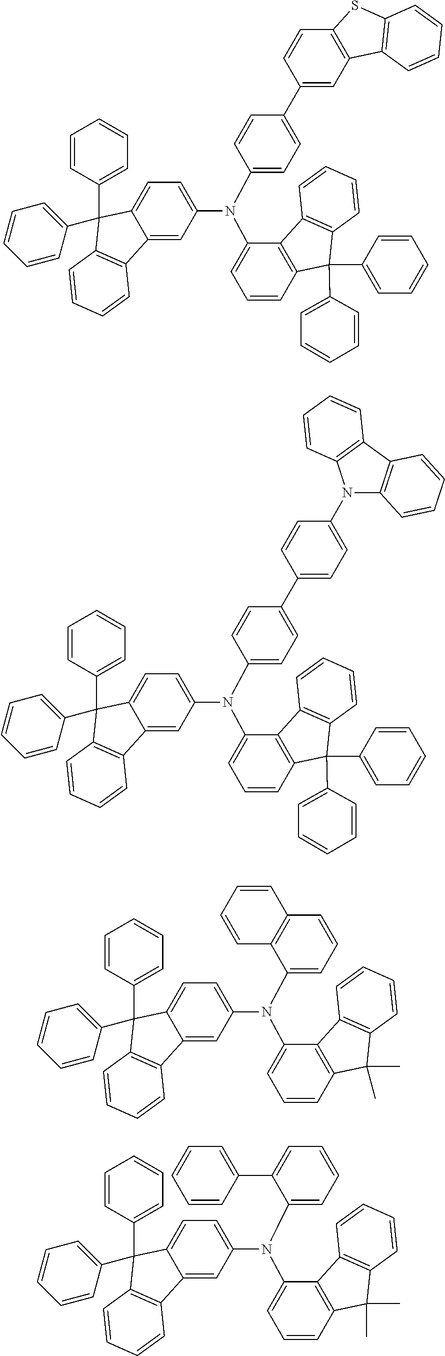

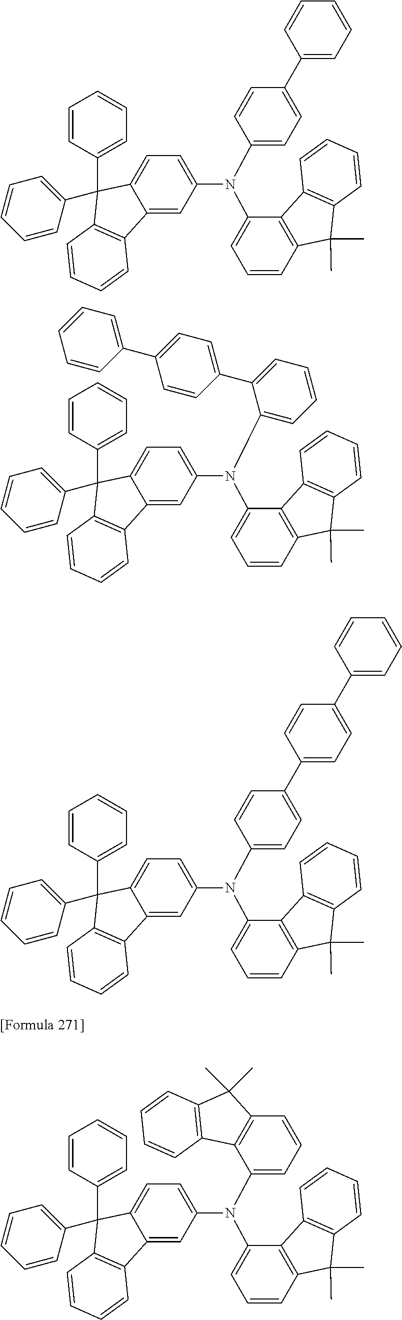

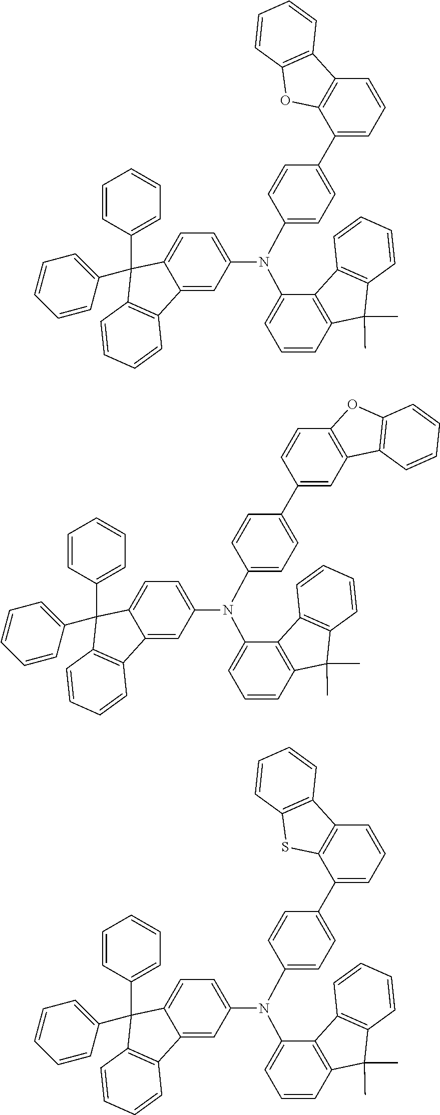 [SMILES: CC1(C)c2ccccc2-c2c(N(c3ccc(-c4cccc5c4oc4ccccc45)cc3)c3ccc4c(c3)-c3ccccc3C4(c3ccccc3)c3ccccc3)cccc21 ; CC1(C)c2ccccc2-c2c(N(c3ccc(-c4ccc5oc6ccccc6c5c4)cc3)c3ccc4c(c3)-c3ccccc3C4(c3ccccc3)c3ccccc3)cccc21 ; CC1(C)c2ccccc2-c2c(N(c3ccc(-c4cccc5c4sc4ccccc45)cc3)c3ccc4c(c3)-c3ccccc3C4(c3ccccc3)c3ccccc3)cccc21]

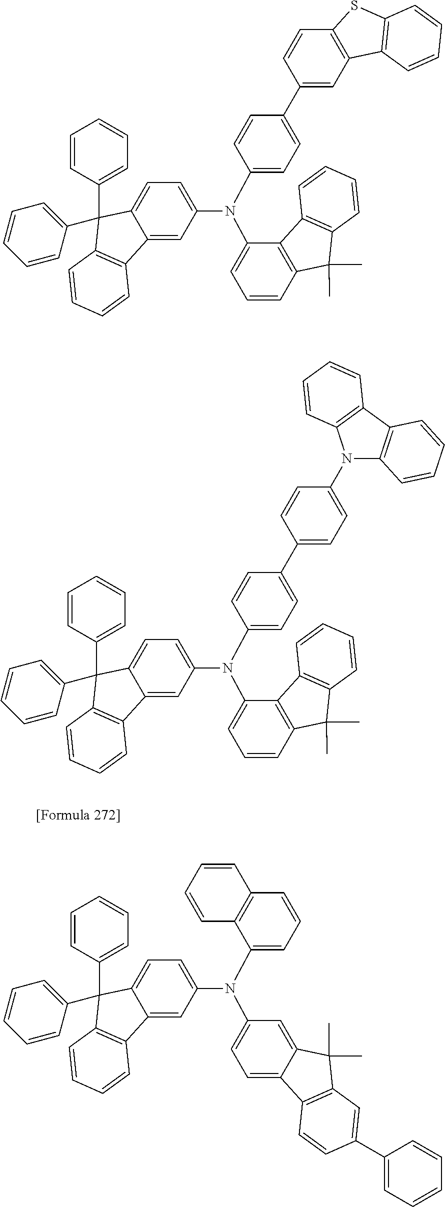 [SMILES: CC1(C)c2ccccc2-c2c(N(c3ccc(-c4ccc5sc6ccccc6c5c4)cc3)c3ccc4c(c3)-c3ccccc3C4(c3ccccc3)c3ccccc3)cccc21 ; CC1(C)c2ccccc2-c2c(N(c3ccc(-c4ccc(-n5c6ccccc6c6ccccc65)cc4)cc3)c3ccc4c(c3)-c3ccccc3C4(c3ccccc3)c3ccccc3)cccc21 ; CC1(C)c2cc(-c3ccccc3)ccc2-c2ccc(N(c3ccc4c(c3)-c3ccccc3C4(c3ccccc3)c3ccccc3)c3cccc4ccccc34)cc21]

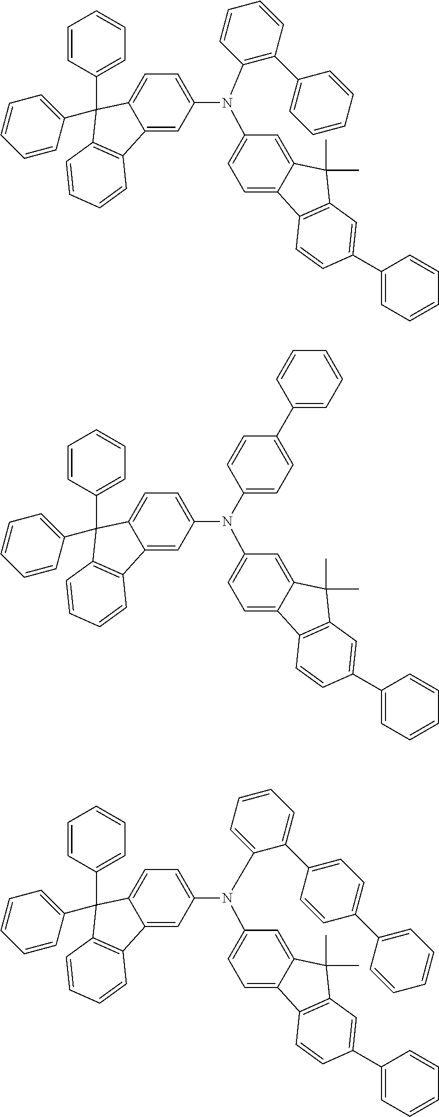 [SMILES: CC1(C)c2cc(-c3ccccc3)ccc2-c2ccc(N(c3ccc4c(c3)-c3ccccc3C4(c3ccccc3)c3ccccc3)c3ccccc3-c3ccccc3)cc21 ; CC1(C)c2cc(-c3ccccc3)ccc2-c2ccc(N(c3ccc(-c4ccccc4)cc3)c3ccc4c(c3)-c3ccccc3C4(c3ccccc3)c3ccccc3)cc21 ; CC1(C)c2cc(-c3ccccc3)ccc2-c2ccc(N(c3ccc4c(c3)-c3ccccc3C4(c3ccccc3)c3ccccc3)c3ccccc3-c3ccc(-c4ccccc4)cc3)cc21]

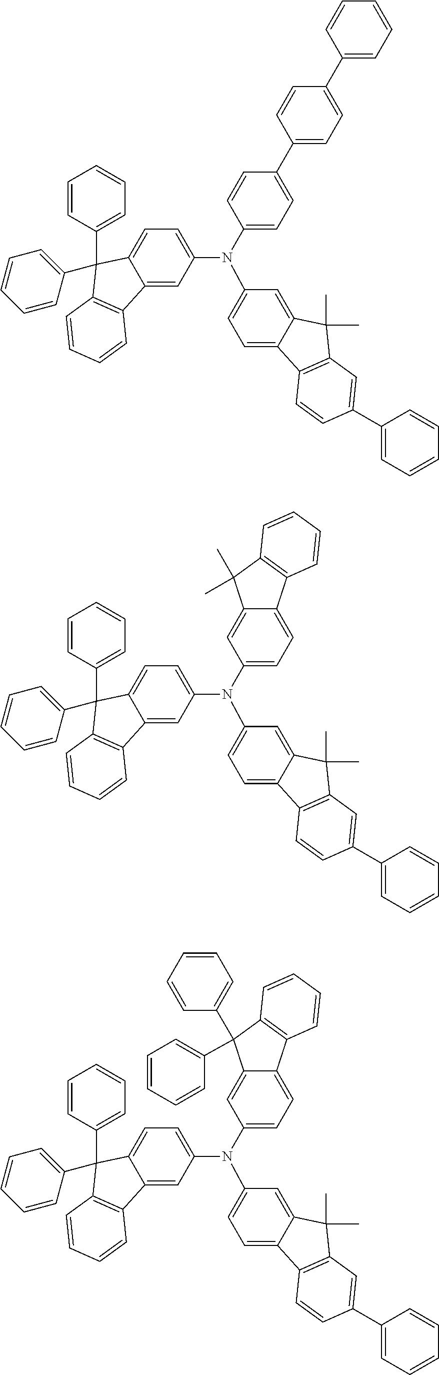 [SMILES: CC1(C)c2cc(-c3ccccc3)ccc2-c2ccc(N(c3ccc(-c4ccc(-c5ccccc5)cc4)cc3)c3ccc4c(c3)-c3ccccc3C4(c3ccccc3)c3ccccc3)cc21 ; CC1(C)c2ccccc2-c2ccc(N(c3ccc4c(c3)-c3ccccc3C4(c3ccccc3)c3ccccc3)c3ccc4c(c3)C(C)(C)c3cc(-c5ccccc5)ccc3-4)cc21 ; CC1(C)c2cc(-c3ccccc3)ccc2-c2ccc(N(c3ccc4c(c3)-c3ccccc3C4(c3ccccc3)c3ccccc3)c3ccc4c(c3)C(c3ccccc3)(c3ccccc3)c3ccccc3-4)cc21]

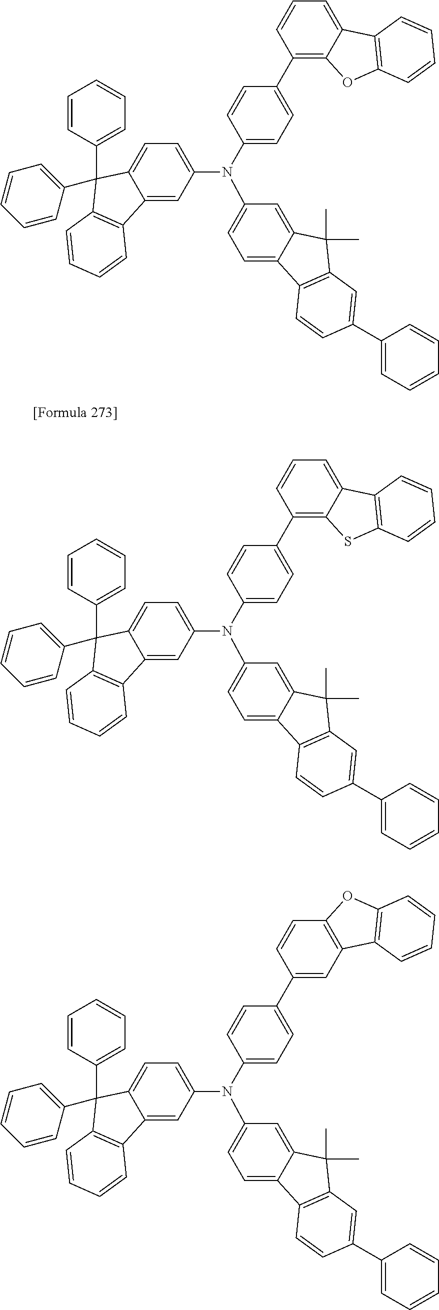 [SMILES: CC1(C)c2cc(-c3ccccc3)ccc2-c2ccc(N(c3ccc(-c4cccc5c4oc4ccccc45)cc3)c3ccc4c(c3)-c3ccccc3C4(c3ccccc3)c3ccccc3)cc21 ; CC1(C)c2cc(-c3ccccc3)ccc2-c2ccc(N(c3ccc(-c4cccc5c4sc4ccccc45)cc3)c3ccc4c(c3)-c3ccccc3C4(c3ccccc3)c3ccccc3)cc21 ; CC1(C)c2cc(-c3ccccc3)ccc2-c2ccc(N(c3ccc(-c4ccc5oc6ccccc6c5c4)cc3)c3ccc4c(c3)-c3ccccc3C4(c3ccccc3)c3ccccc3)cc21]

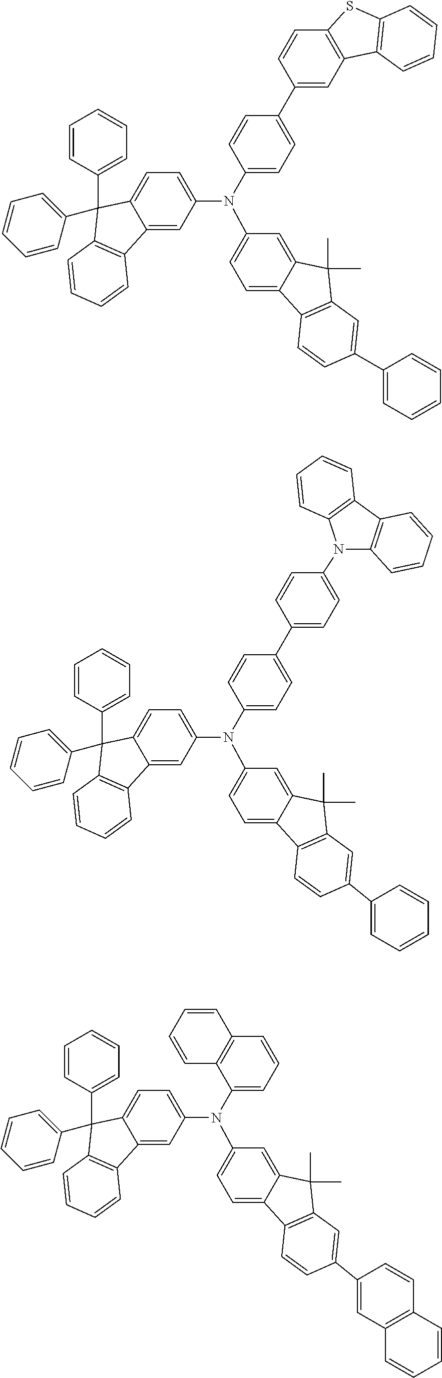 [SMILES: CC1(C)c2cc(-c3ccccc3)ccc2-c2ccc(N(c3ccc(-c4ccc5sc6ccccc6c5c4)cc3)c3ccc4c(c3)-c3ccccc3C4(c3ccccc3)c3ccccc3)cc21 ; CC1(C)c2cc(-c3ccccc3)ccc2-c2ccc(N(c3ccc(-c4ccc(-n5c6ccccc6c6ccccc65)cc4)cc3)c3ccc4c(c3)-c3ccccc3C4(c3ccccc3)c3ccccc3)cc21 ; CC1(C)c2cc(-c3ccc4ccccc4c3)ccc2-c2ccc(N(c3ccc4c(c3)-c3ccccc3C4(c3ccccc3)c3ccccc3)c3cccc4ccccc34)cc21]

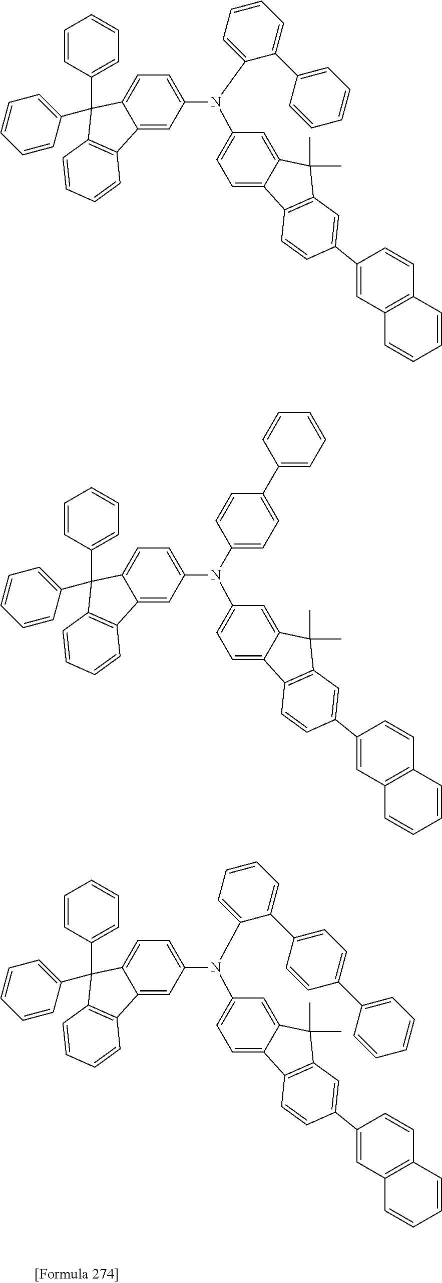 [SMILES: CC1(C)c2cc(-c3ccc4ccccc4c3)ccc2-c2ccc(N(c3ccc4c(c3)-c3ccccc3C4(c3ccccc3)c3ccccc3)c3ccccc3-c3ccccc3)cc21 ; CC1(C)c2cc(-c3ccc4ccccc4c3)ccc2-c2ccc(N(c3ccc(-c4ccccc4)cc3)c3ccc4c(c3)-c3ccccc3C4(c3ccccc3)c3ccccc3)cc21 ; CC1(C)c2cc(-c3ccc4ccccc4c3)ccc2-c2ccc(N(c3ccc4c(c3)-c3ccccc3C4(c3ccccc3)c3ccccc3)c3ccccc3-c3ccc(-c4ccccc4)cc3)cc21]

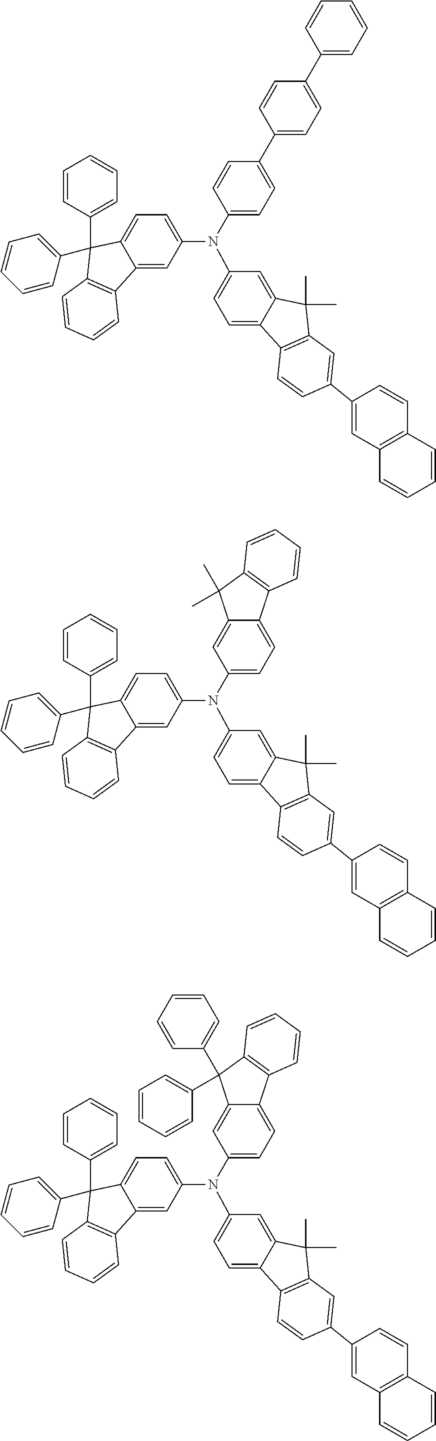 [SMILES: CC1(C)c2cc(-c3ccc4ccccc4c3)ccc2-c2ccc(N(c3ccc(-c4ccc(-c5ccccc5)cc4)cc3)c3ccc4c(c3)-c3ccccc3C4(c3ccccc3)c3ccccc3)cc21 ; CC1(C)c2ccccc2-c2ccc(N(c3ccc4c(c3)-c3ccccc3C4(c3ccccc3)c3ccccc3)c3ccc4c(c3)C(C)(C)c3cc(-c5ccc6ccccc6c5)ccc3-4)cc21 ; CC1(C)c2cc(-c3ccc4ccccc4c3)ccc2-c2ccc(N(c3ccc4c(c3)-c3ccccc3C4(c3ccccc3)c3ccccc3)c3ccc4c(c3)C(c3ccccc3)(c3ccccc3)c3ccccc3-4)cc21]

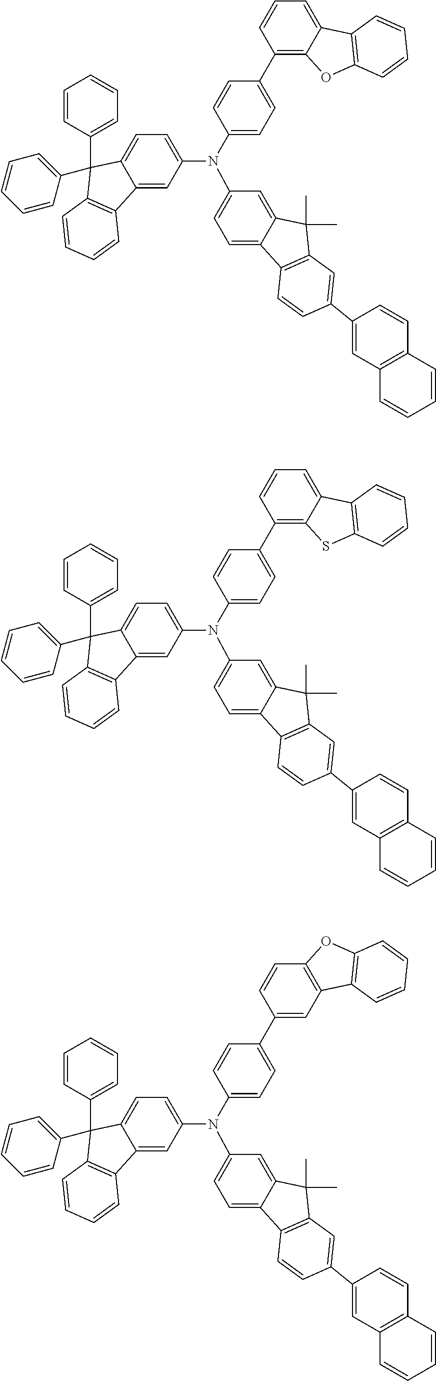 [SMILES: CC1(C)c2cc(-c3ccc4ccccc4c3)ccc2-c2ccc(N(c3ccc(-c4cccc5c4oc4ccccc45)cc3)c3ccc4c(c3)-c3ccccc3C4(c3ccccc3)c3ccccc3)cc21 ; CC1(C)c2cc(-c3ccc4ccccc4c3)ccc2-c2ccc(N(c3ccc(-c4cccc5c4sc4ccccc45)cc3)c3ccc4c(c3)-c3ccccc3C4(c3ccccc3)c3ccccc3)cc21 ; CC1(C)c2cc(-c3ccc4ccccc4c3)ccc2-c2ccc(N(c3ccc(-c4ccc5oc6ccccc6c5c4)cc3)c3ccc4c(c3)-c3ccccc3C4(c3ccccc3)c3ccccc3)cc21]

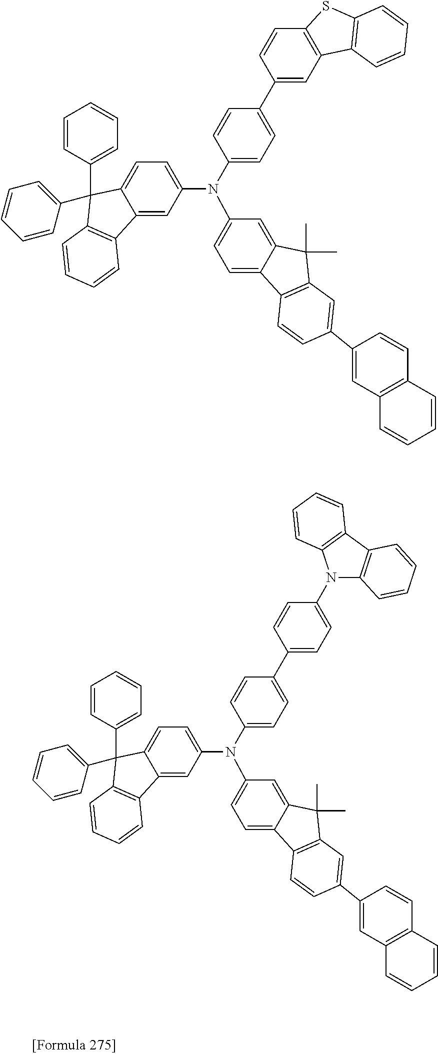 [SMILES: CC1(C)c2cc(-c3ccc4ccccc4c3)ccc2-c2ccc(N(c3ccc(-c4ccc5sc6ccccc6c5c4)cc3)c3ccc4c(c3)-c3ccccc3C4(c3ccccc3)c3ccccc3)cc21 ; CC1(C)c2cc(-c3ccc4ccccc4c3)ccc2-c2ccc(N(c3ccc(-c4ccc(-n5c6ccccc6c6ccccc65)cc4)cc3)c3ccc4c(c3)-c3ccccc3C4(c3ccccc3)c3ccccc3)cc21]

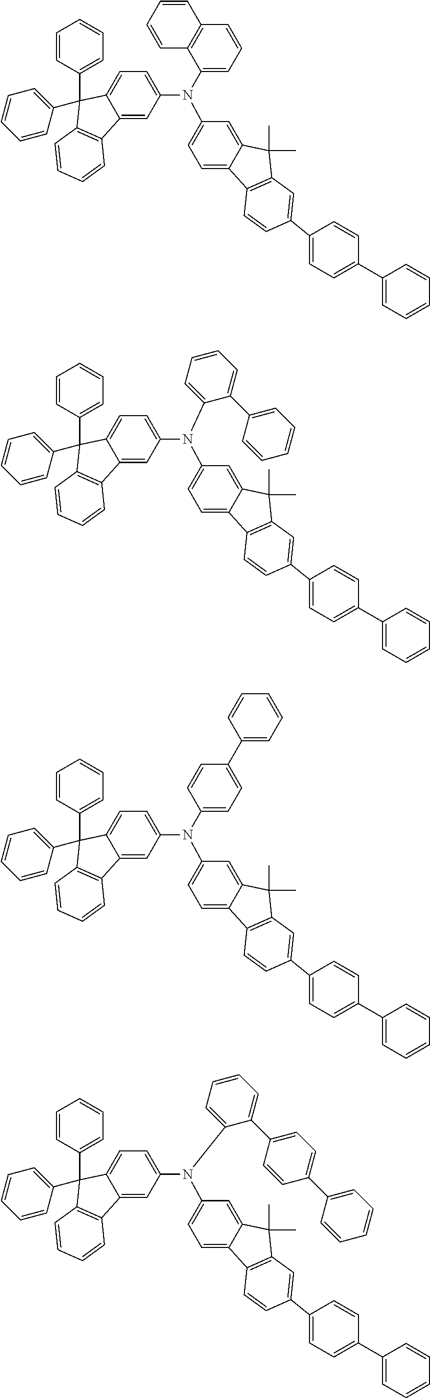 [SMILES: CC1(C)c2cc(-c3ccc(-c4ccccc4)cc3)ccc2-c2ccc(N(c3ccc4c(c3)-c3ccccc3C4(c3ccccc3)c3ccccc3)c3cccc4ccccc34)cc21 ; CC1(C)c2cc(-c3ccc(-c4ccccc4)cc3)ccc2-c2ccc(N(c3ccc4c(c3)-c3ccccc3C4(c3ccccc3)c3ccccc3)c3ccccc3-c3ccccc3)cc21 ; CC1(C)c2cc(-c3ccc(-c4ccccc4)cc3)ccc2-c2ccc(N(c3ccc(-c4ccccc4)cc3)c3ccc4c(c3)-c3ccccc3C4(c3ccccc3)c3ccccc3)cc21 ; CC1(C)c2cc(-c3ccc(-c4ccccc4)cc3)ccc2-c2ccc(N(c3ccc4c(c3)-c3ccccc3C4(c3ccccc3)c3ccccc3)c3ccccc3-c3ccc(-c4ccccc4)cc3)cc21]

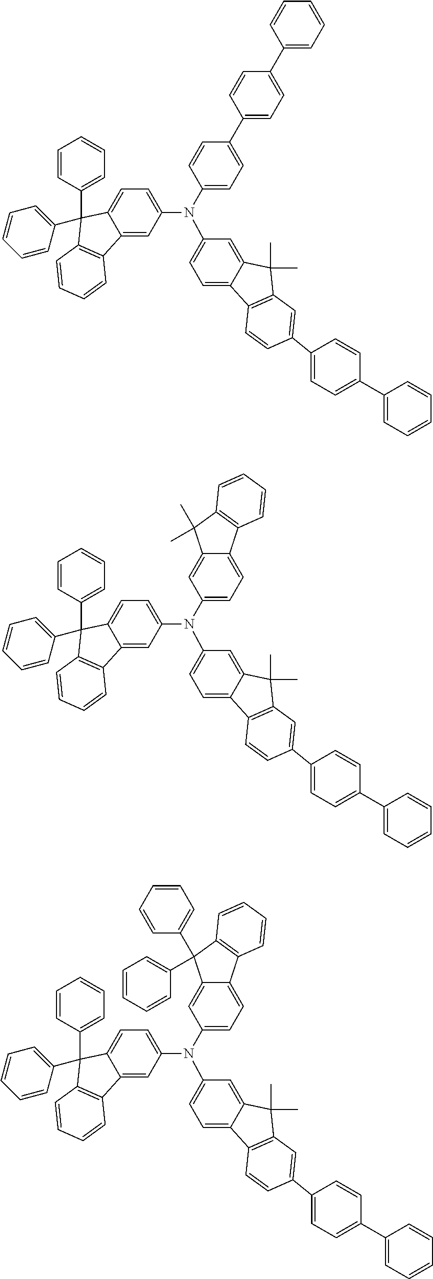 [SMILES: CC1(C)c2cc(-c3ccc(-c4ccccc4)cc3)ccc2-c2ccc(N(c3ccc(-c4ccc(-c5ccccc5)cc4)cc3)c3ccc4c(c3)-c3ccccc3C4(c3ccccc3)c3ccccc3)cc21 ; CC1(C)c2ccccc2-c2ccc(N(c3ccc4c(c3)-c3ccccc3C4(c3ccccc3)c3ccccc3)c3ccc4c(c3)C(C)(C)c3cc(-c5ccc(-c6ccccc6)cc5)ccc3-4)cc21 ; CC1(C)c2cc(-c3ccc(-c4ccccc4)cc3)ccc2-c2ccc(N(c3ccc4c(c3)-c3ccccc3C4(c3ccccc3)c3ccccc3)c3ccc4c(c3)C(c3ccccc3)(c3ccccc3)c3ccccc3-4)cc21]

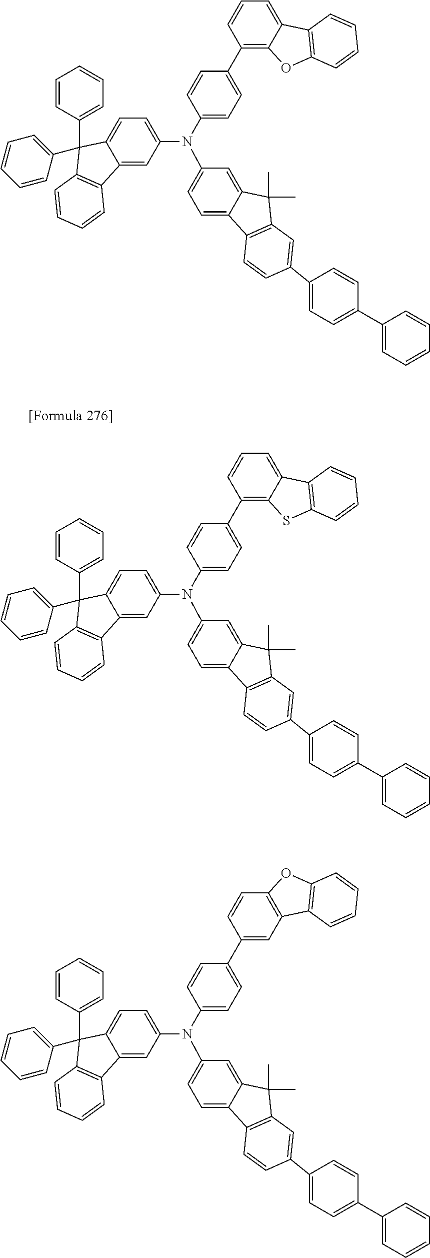 [SMILES: CC1(C)c2cc(-c3ccc(-c4ccccc4)cc3)ccc2-c2ccc(N(c3ccc(-c4cccc5c4oc4ccccc45)cc3)c3ccc4c(c3)-c3ccccc3C4(c3ccccc3)c3ccccc3)cc21 ; CC1(C)c2cc(-c3ccc(-c4ccccc4)cc3)ccc2-c2ccc(N(c3ccc(-c4cccc5c4sc4ccccc45)cc3)c3ccc4c(c3)-c3ccccc3C4(c3ccccc3)c3ccccc3)cc21 ; CC1(C)c2cc(-c3ccc(-c4ccccc4)cc3)ccc2-c2ccc(N(c3ccc(-c4ccc5oc6ccccc6c5c4)cc3)c3ccc4c(c3)-c3ccccc3C4(c3ccccc3)c3ccccc3)cc21]

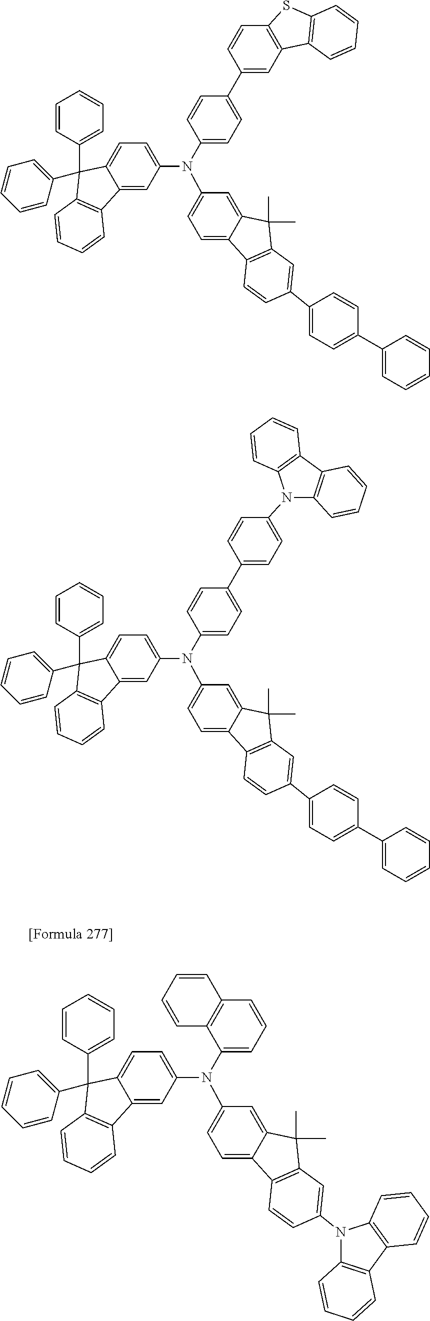 [SMILES: CC1(C)c2cc(-c3ccc(-c4ccccc4)cc3)ccc2-c2ccc(N(c3ccc(-c4ccc5sc6ccccc6c5c4)cc3)c3ccc4c(c3)-c3ccccc3C4(c3ccccc3)c3ccccc3)cc21 ; CC1(C)c2cc(-c3ccc(-c4ccccc4)cc3)ccc2-c2ccc(N(c3ccc(-c4ccc(-n5c6ccccc6c6ccccc65)cc4)cc3)c3ccc4c(c3)-c3ccccc3C4(c3ccccc3)c3ccccc3)cc21 ; CC1(C)c2cc(N(c3ccc4c(c3)-c3ccccc3C4(c3ccccc3)c3ccccc3)c3cccc4ccccc34)ccc2-c2ccc(-n3c4ccccc4c4ccccc43)cc21]

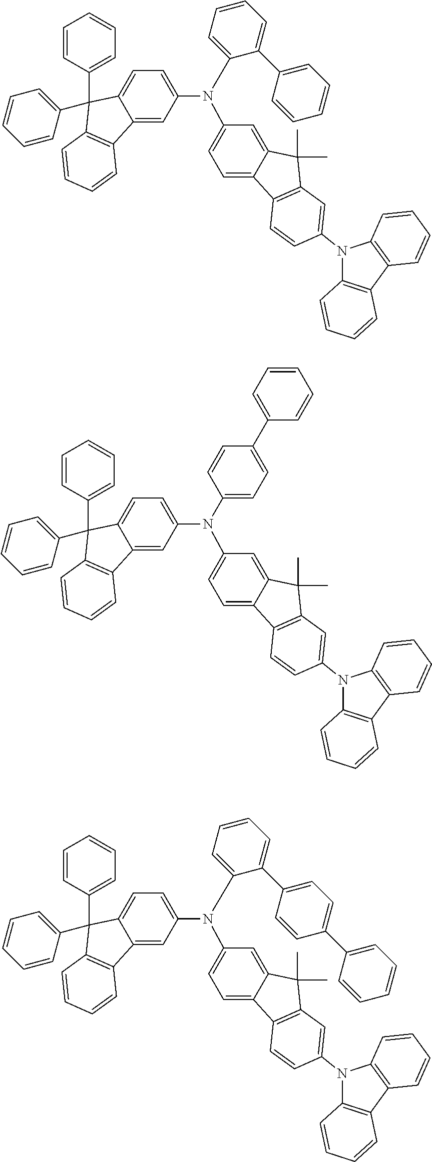 [SMILES: CC1(C)c2cc(N(c3ccc4c(c3)-c3ccccc3C4(c3ccccc3)c3ccccc3)c3ccccc3-c3ccccc3)ccc2-c2ccc(-n3c4ccccc4c4ccccc43)cc21 ; CC1(C)c2cc(N(c3ccc(-c4ccccc4)cc3)c3ccc4c(c3)-c3ccccc3C4(c3ccccc3)c3ccccc3)ccc2-c2ccc(-n3c4ccccc4c4ccccc43)cc21 ; CC1(C)c2cc(N(c3ccc4c(c3)-c3ccccc3C4(c3ccccc3)c3ccccc3)c3ccccc3-c3ccc(-c4ccccc4)cc3)ccc2-c2ccc(-n3c4ccccc4c4ccccc43)cc21]

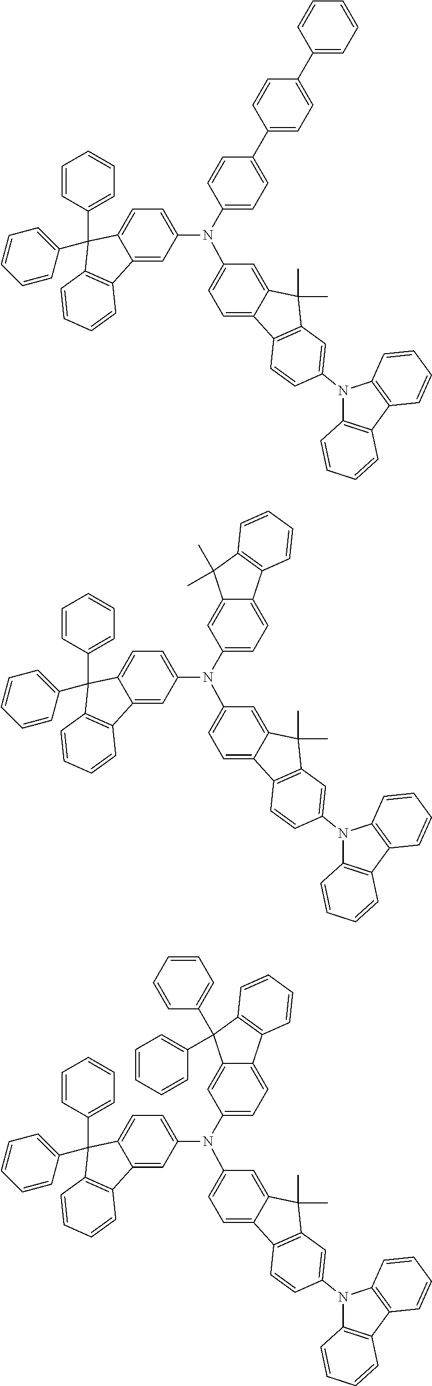 [SMILES: CC1(C)c2cc(N(c3ccc(-c4ccc(-c5ccccc5)cc4)cc3)c3ccc4c(c3)-c3ccccc3C4(c3ccccc3)c3ccccc3)ccc2-c2ccc(-n3c4ccccc4c4ccccc43)cc21 ; CC1(C)c2ccccc2-c2ccc(N(c3ccc4c(c3)-c3ccccc3C4(c3ccccc3)c3ccccc3)c3ccc4c(c3)C(C)(C)c3cc(-n5c6ccccc6c6ccccc65)ccc3-4)cc21 ; CC1(C)c2cc(N(c3ccc4c(c3)-c3ccccc3C4(c3ccccc3)c3ccccc3)c3ccc4c(c3)C(c3ccccc3)(c3ccccc3)c3ccccc3-4)ccc2-c2ccc(-n3c4ccccc4c4ccccc43)cc21]

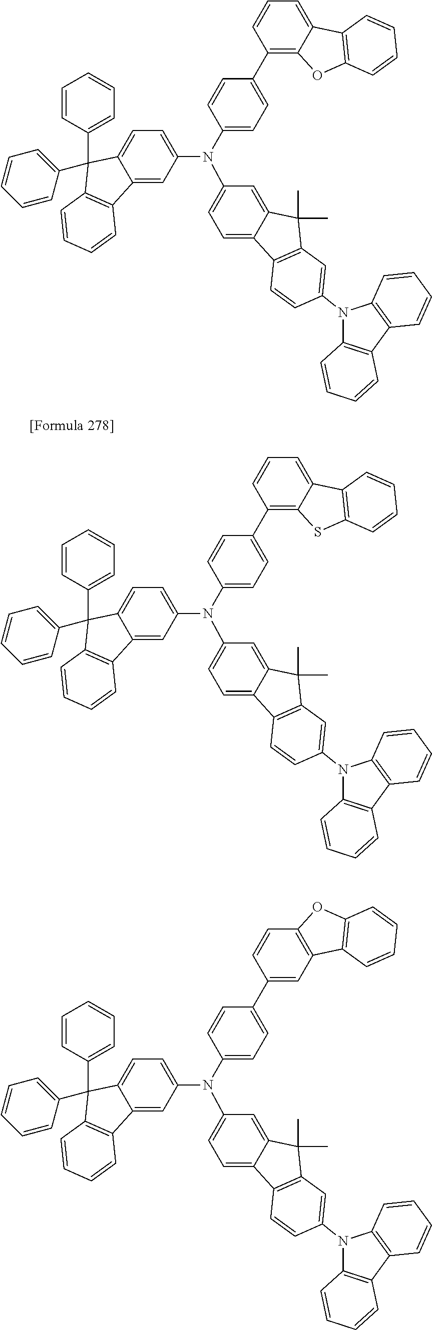 [SMILES: CC1(C)c2cc(N(c3ccc(-c4cccc5c4oc4ccccc45)cc3)c3ccc4c(c3)-c3ccccc3C4(c3ccccc3)c3ccccc3)ccc2-c2ccc(-n3c4ccccc4c4ccccc43)cc21 ; CC1(C)c2cc(N(c3ccc(-c4cccc5c4sc4ccccc45)cc3)c3ccc4c(c3)-c3ccccc3C4(c3ccccc3)c3ccccc3)ccc2-c2ccc(-n3c4ccccc4c4ccccc43)cc21 ; CC1(C)c2cc(N(c3ccc(-c4ccc5oc6ccccc6c5c4)cc3)c3ccc4c(c3)-c3ccccc3C4(c3ccccc3)c3ccccc3)ccc2-c2ccc(-n3c4ccccc4c4ccccc43)cc21]

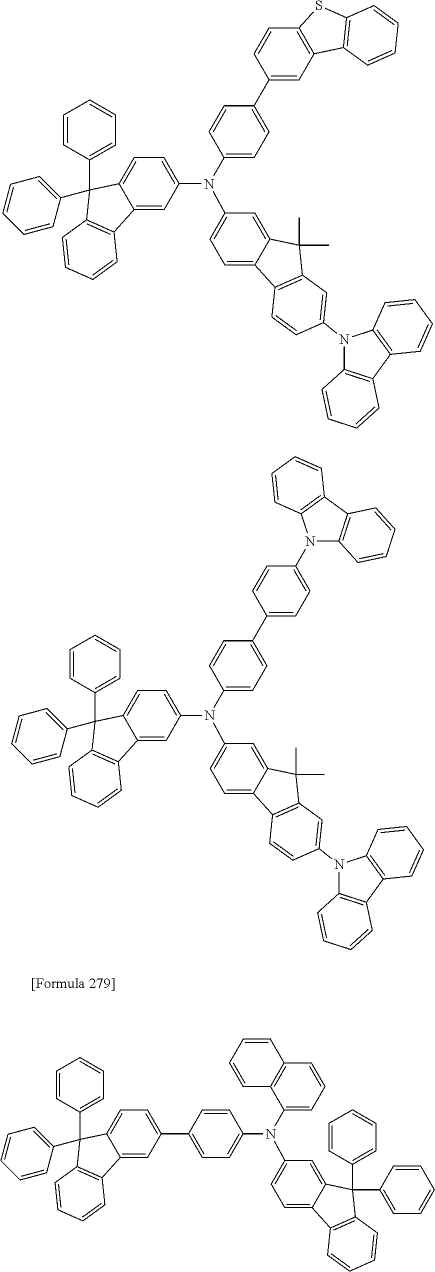 [SMILES: CC1(C)c2cc(N(c3ccc(-c4ccc5sc6ccccc6c5c4)cc3)c3ccc4c(c3)-c3ccccc3C4(c3ccccc3)c3ccccc3)ccc2-c2ccc(-n3c4ccccc4c4ccccc43)cc21 ; CC1(C)c2cc(N(c3ccc(-c4ccc(-n5c6ccccc6c6ccccc65)cc4)cc3)c3ccc4c(c3)-c3ccccc3C4(c3ccccc3)c3ccccc3)ccc2-c2ccc(-n3c4ccccc4c4ccccc43)cc21 ; c1ccc(C2(c3ccccc3)c3ccccc3-c3cc(-c4ccc(N(c5ccc6c(c5)C(c5ccccc5)(c5ccccc5)c5ccccc5-6)c5cccc6ccccc56)cc4)ccc32)cc1]

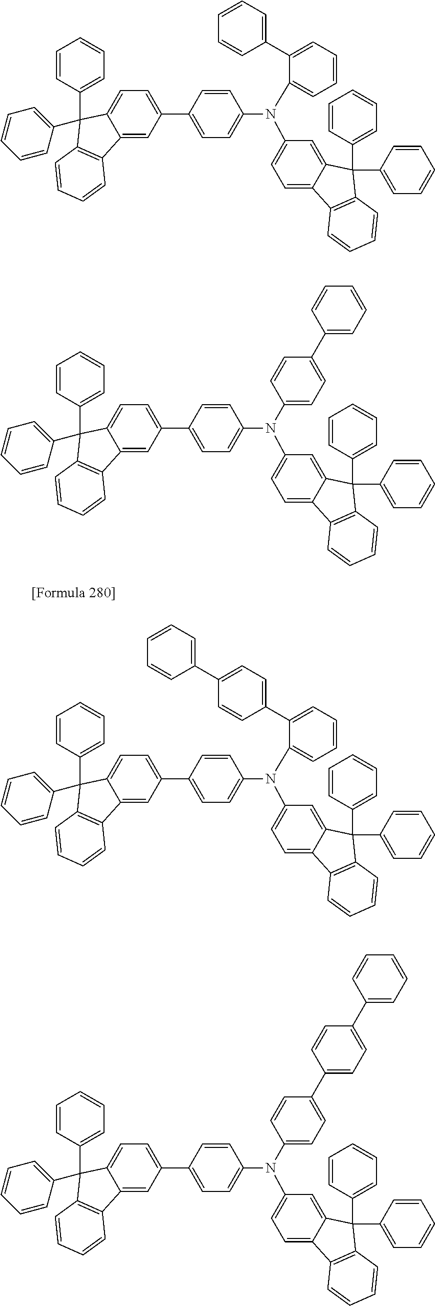

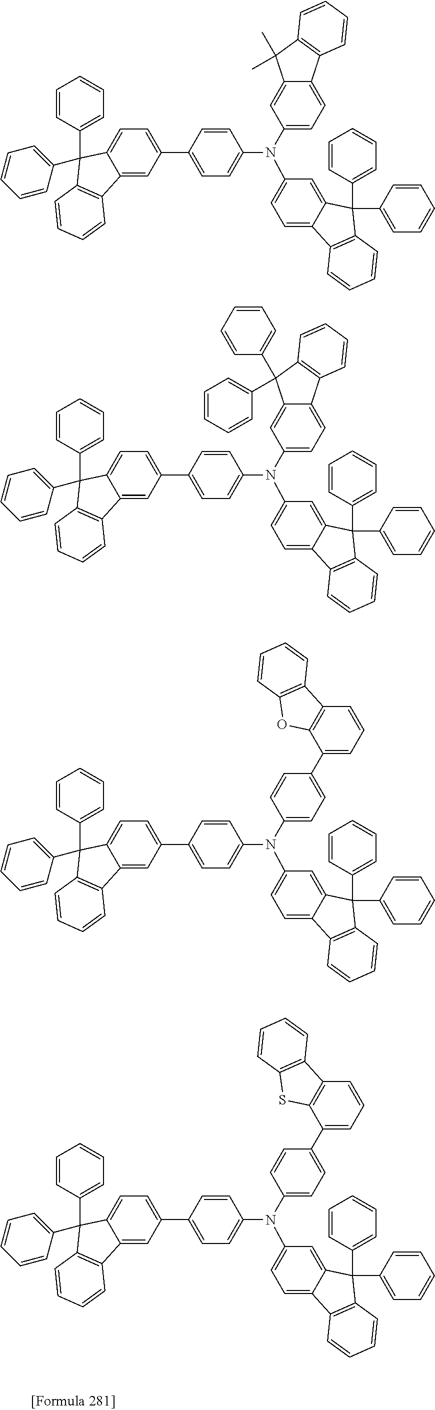

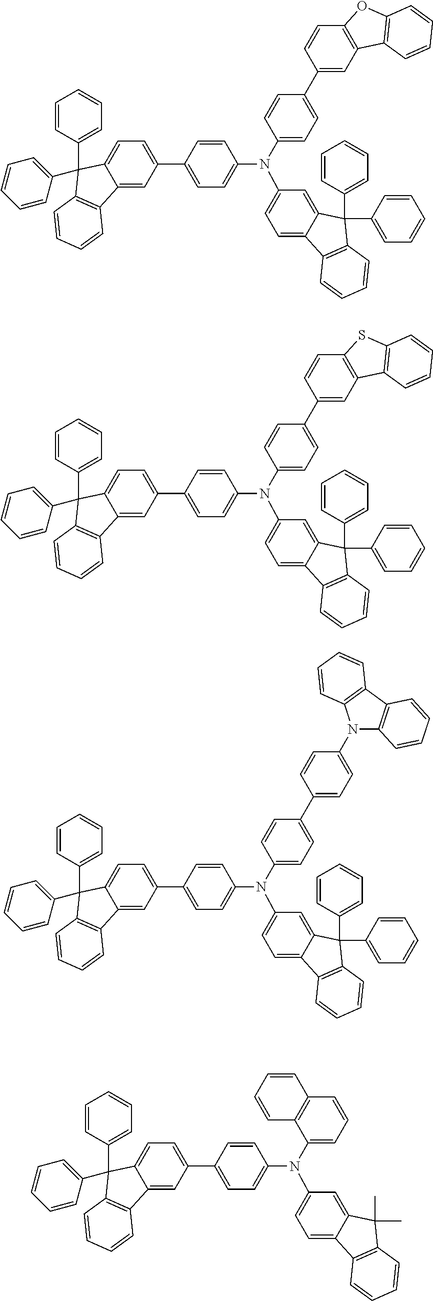

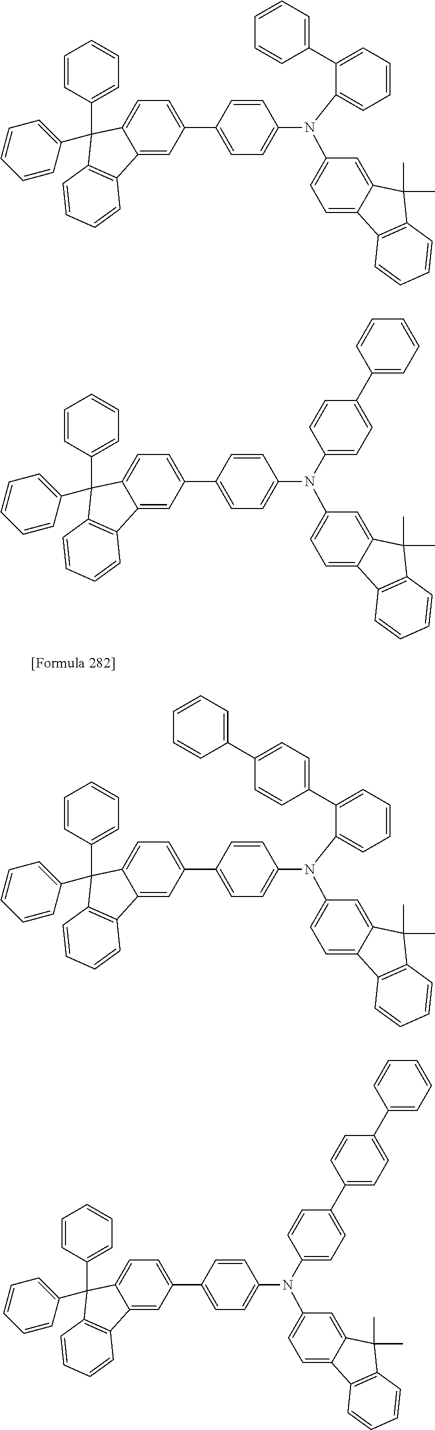

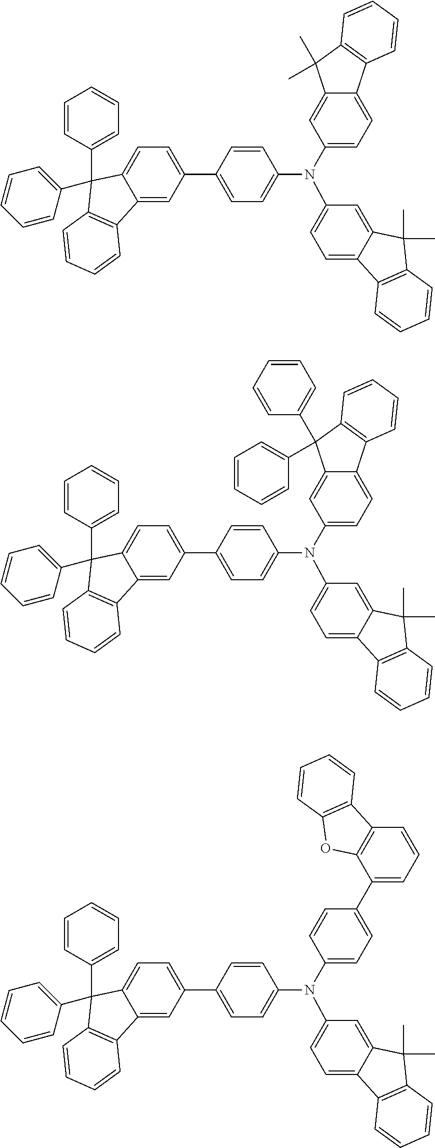 [SMILES: CC1(C)c2ccccc2-c2ccc(N(c3ccc(-c4ccc5c(c4)-c4ccccc4C5(c4ccccc4)c4ccccc4)cc3)c3ccc4c(c3)C(C)(C)c3ccccc3-4)cc21 ; CC1(C)c2ccccc2-c2ccc(N(c3ccc(-c4ccc5c(c4)-c4ccccc4C5(c4ccccc4)c4ccccc4)cc3)c3ccc4c(c3)C(c3ccccc3)(c3ccccc3)c3ccccc3-4)cc21 ; CC1(C)c2ccccc2-c2ccc(N(c3ccc(-c4ccc5c(c4)-c4ccccc4C5(c4ccccc4)c4ccccc4)cc3)c3ccc(-c4cccc5c4oc4ccccc45)cc3)cc21]

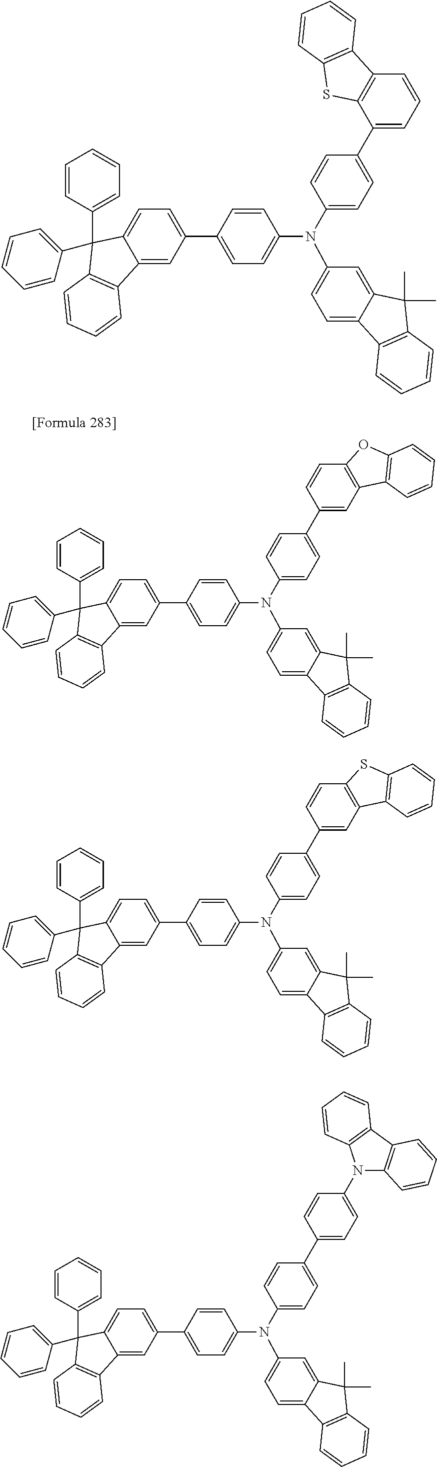

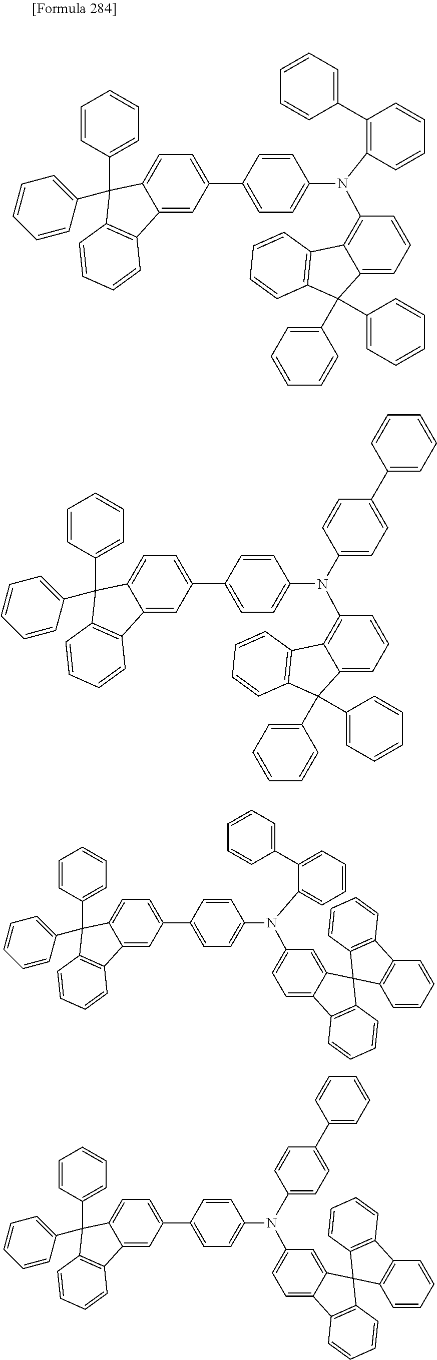

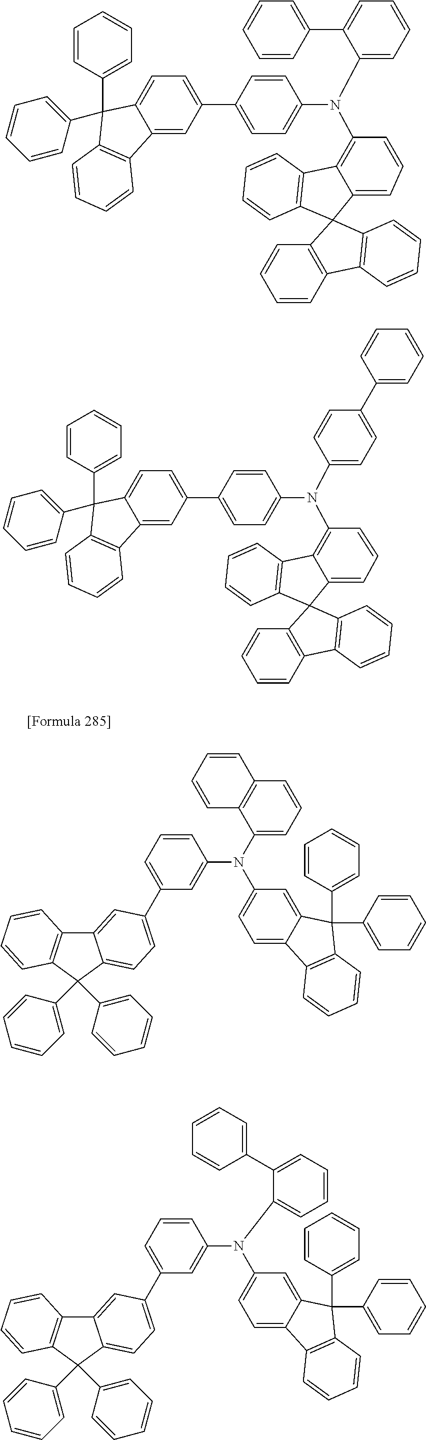 [SMILES: c1ccc(-c2ccccc2N(c2ccc(-c3ccc4c(c3)-c3ccccc3C4(c3ccccc3)c3ccccc3)cc2)c2cccc3c2-c2ccccc2C32c3ccccc3-c3ccccc32)cc1 ; c1ccc(-c2ccc(N(c3ccc(-c4ccc5c(c4)-c4ccccc4C5(c4ccccc4)c4ccccc4)cc3)c3cccc4c3-c3ccccc3C43c4ccccc4-c4ccccc43)cc2)cc1 ; c1ccc(C2(c3ccccc3)c3ccccc3-c3cc(-c4cccc(N(c5ccc6c(c5)C(c5ccccc5)(c5ccccc5)c5ccccc5-6)c5cccc6ccccc56)c4)ccc32)cc1 ; c1ccc(-c2ccccc2N(c2cccc(-c3ccc4c(c3)-c3ccccc3C4(c3ccccc3)c3ccccc3)c2)c2ccc3c(c2)C(c2ccccc2)(c2ccccc2)c2ccccc2-3)cc1]

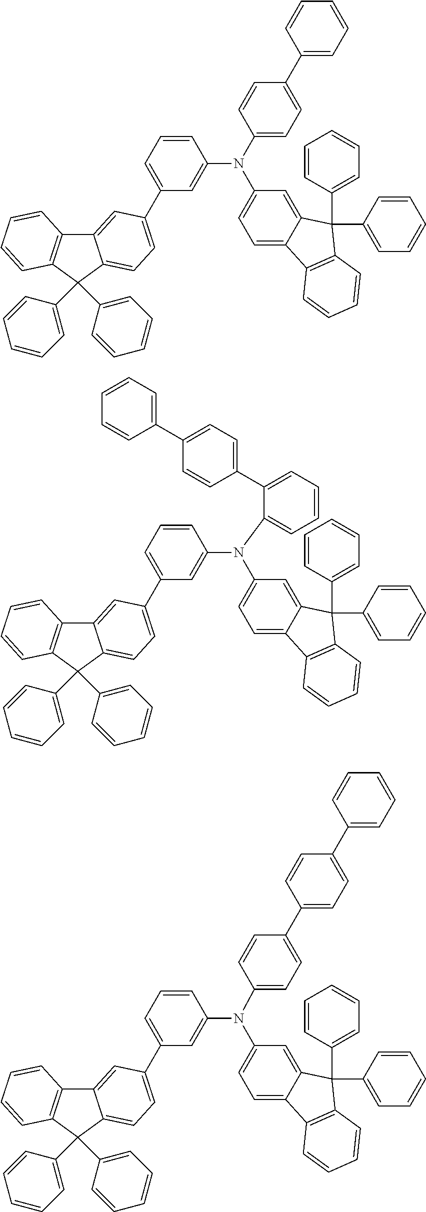

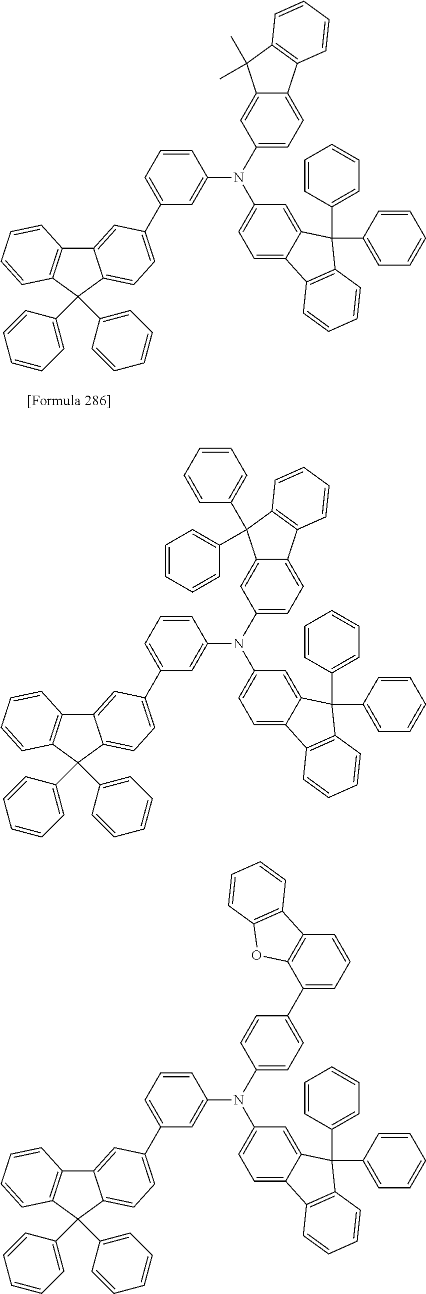 [SMILES: CC1(C)c2ccccc2-c2ccc(N(c3cccc(-c4ccc5c(c4)-c4ccccc4C5(c4ccccc4)c4ccccc4)c3)c3ccc4c(c3)C(c3ccccc3)(c3ccccc3)c3ccccc3-4)cc21 ; c1ccc(C2(c3ccccc3)c3ccccc3-c3cc(-c4cccc(N(c5ccc6c(c5)C(c5ccccc5)(c5ccccc5)c5ccccc5-6)c5ccc6c(c5)C(c5ccccc5)(c5ccccc5)c5ccccc5-6)c4)ccc32)cc1 ; c1ccc(C2(c3ccccc3)c3ccccc3-c3cc(-c4cccc(N(c5ccc(-c6cccc7c6oc6ccccc67)cc5)c5ccc6c(c5)C(c5ccccc5)(c5ccccc5)c5ccccc5-6)c4)ccc32)cc1]

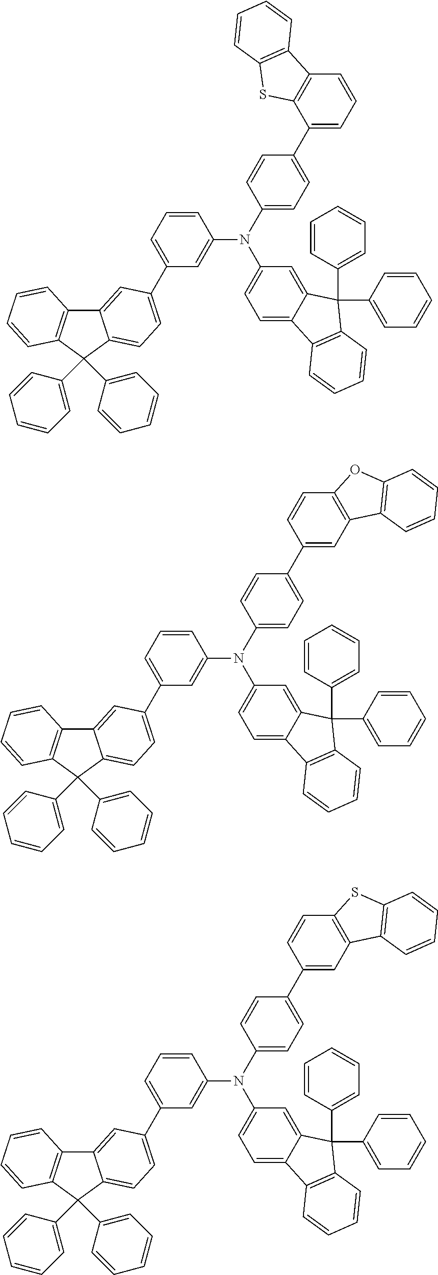 [SMILES: c1ccc(C2(c3ccccc3)c3ccccc3-c3cc(-c4cccc(N(c5ccc(-c6cccc7c6sc6ccccc67)cc5)c5ccc6c(c5)C(c5ccccc5)(c5ccccc5)c5ccccc5-6)c4)ccc32)cc1 ; c1ccc(C2(c3ccccc3)c3ccccc3-c3cc(-c4cccc(N(c5ccc(-c6ccc7oc8ccccc8c7c6)cc5)c5ccc6c(c5)C(c5ccccc5)(c5ccccc5)c5ccccc5-6)c4)ccc32)cc1 ; c1ccc(C2(c3ccccc3)c3ccccc3-c3cc(-c4cccc(N(c5ccc(-c6ccc7sc8ccccc8c7c6)cc5)c5ccc6c(c5)C(c5ccccc5)(c5ccccc5)c5ccccc5-6)c4)ccc32)cc1]

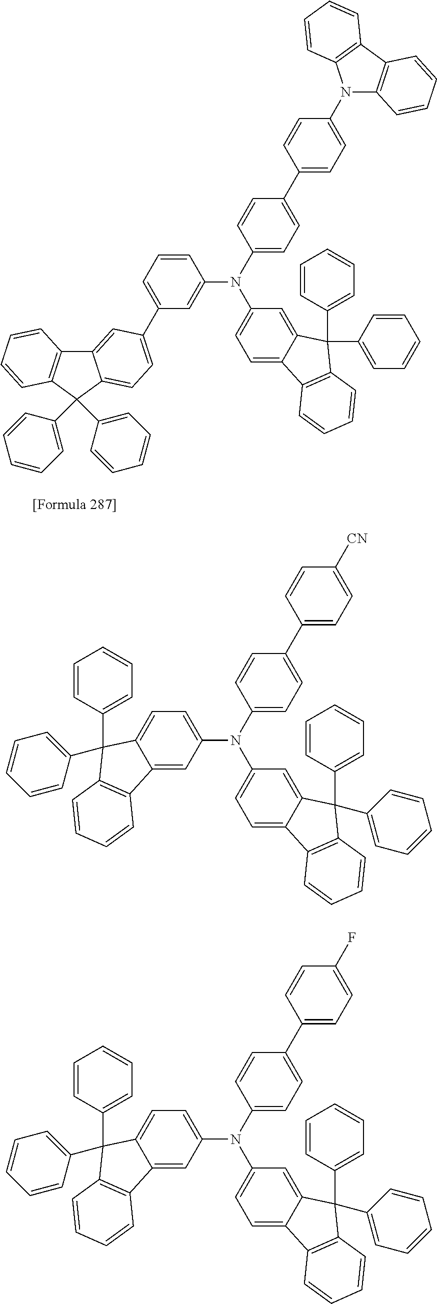

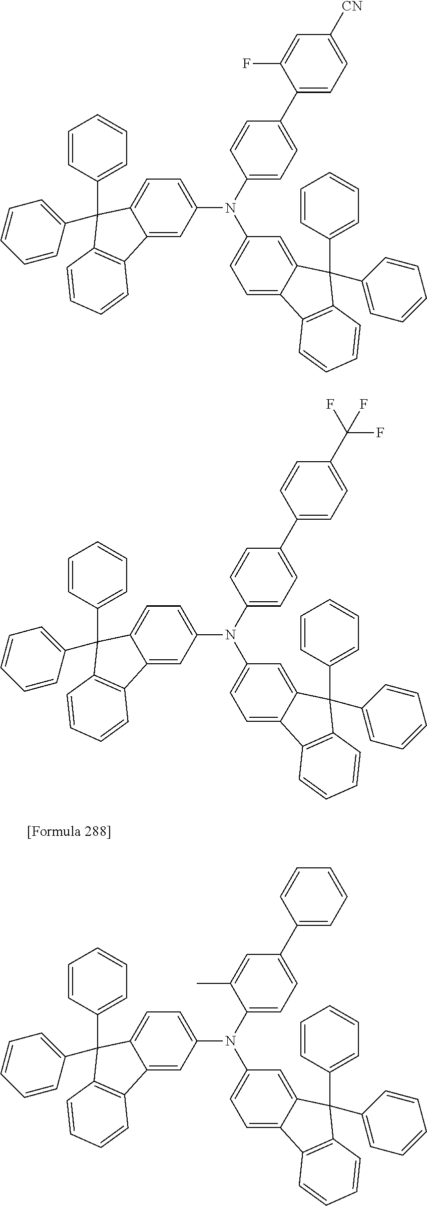

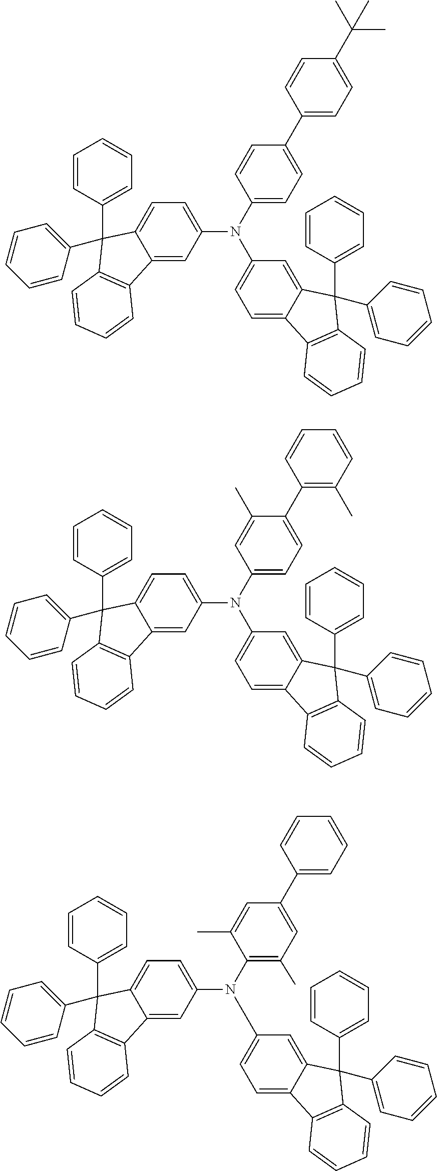

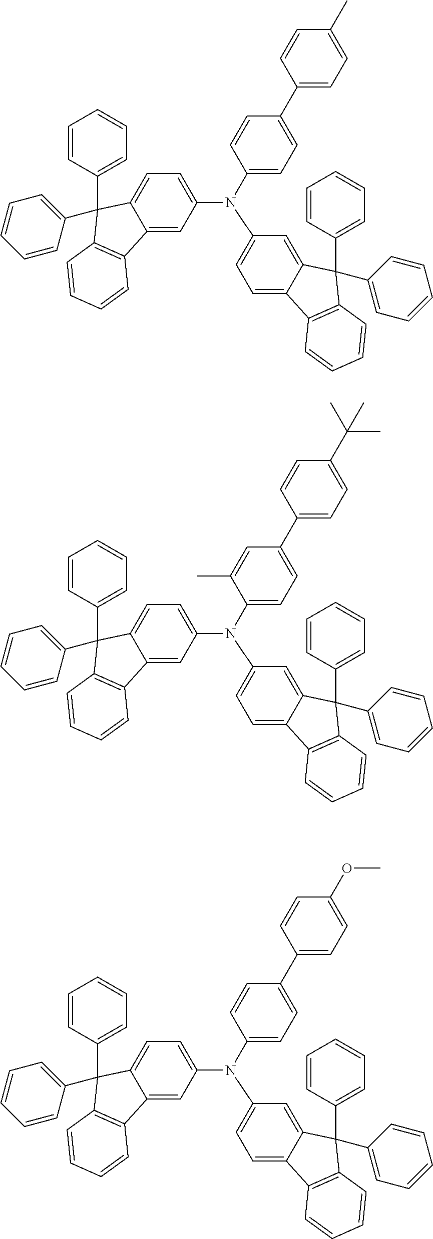

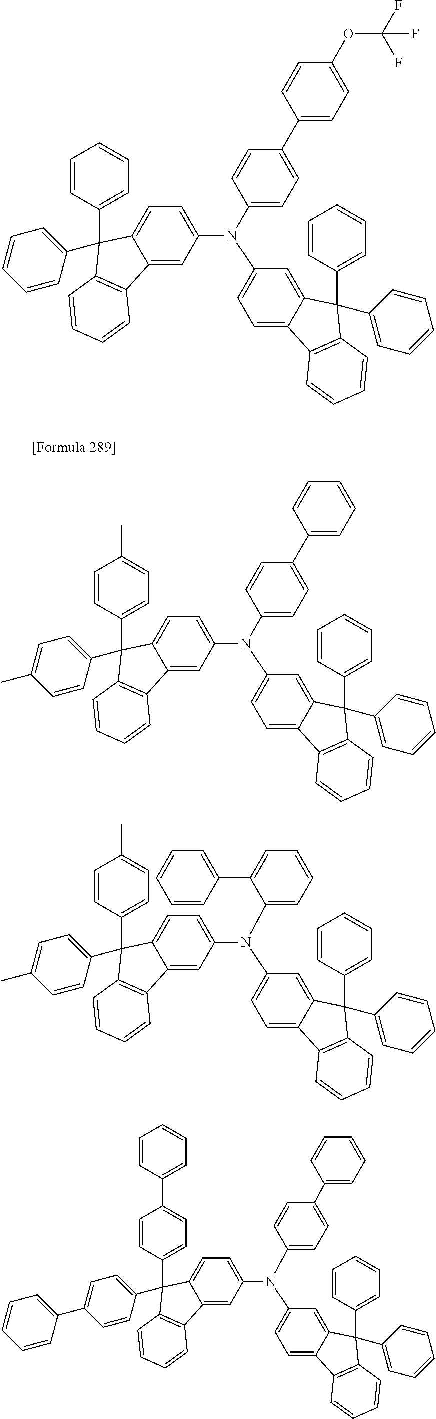

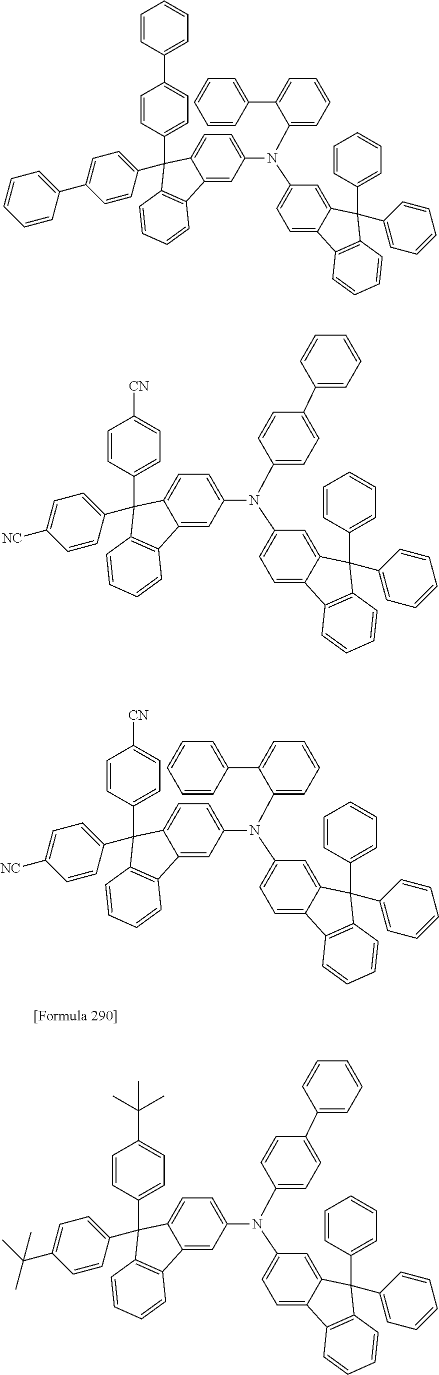

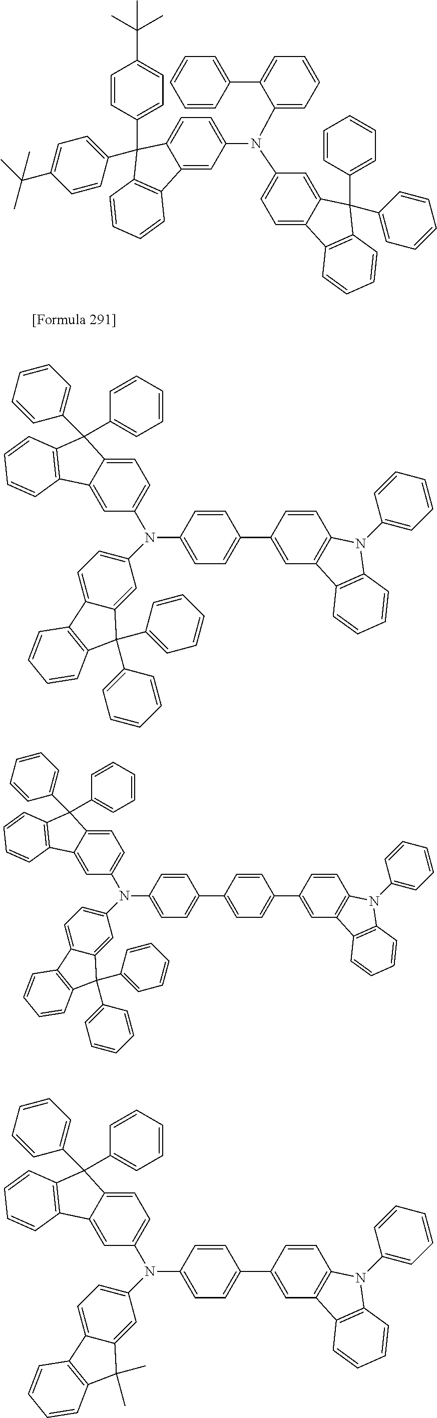

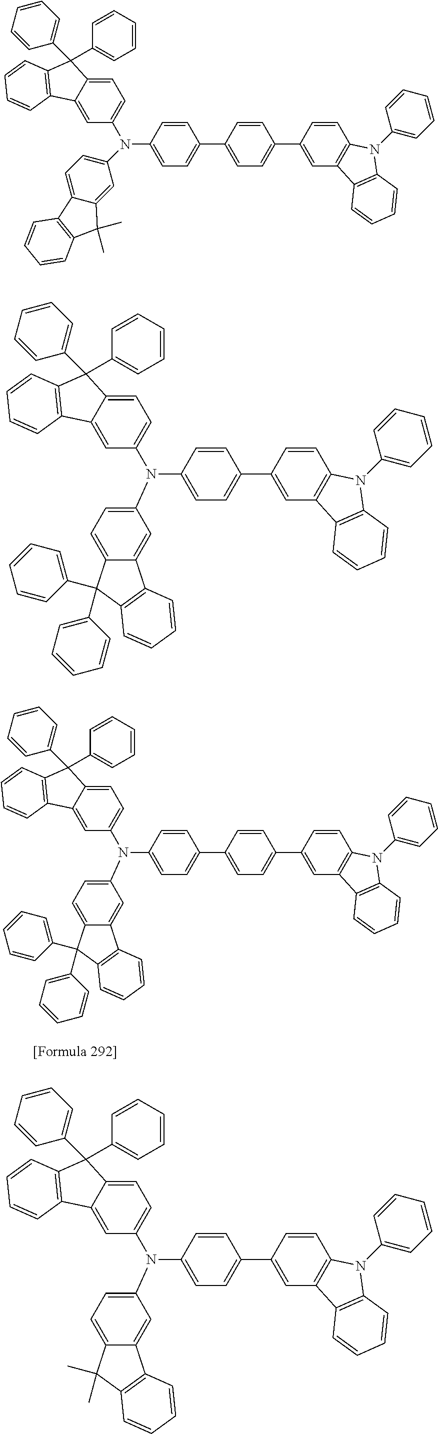

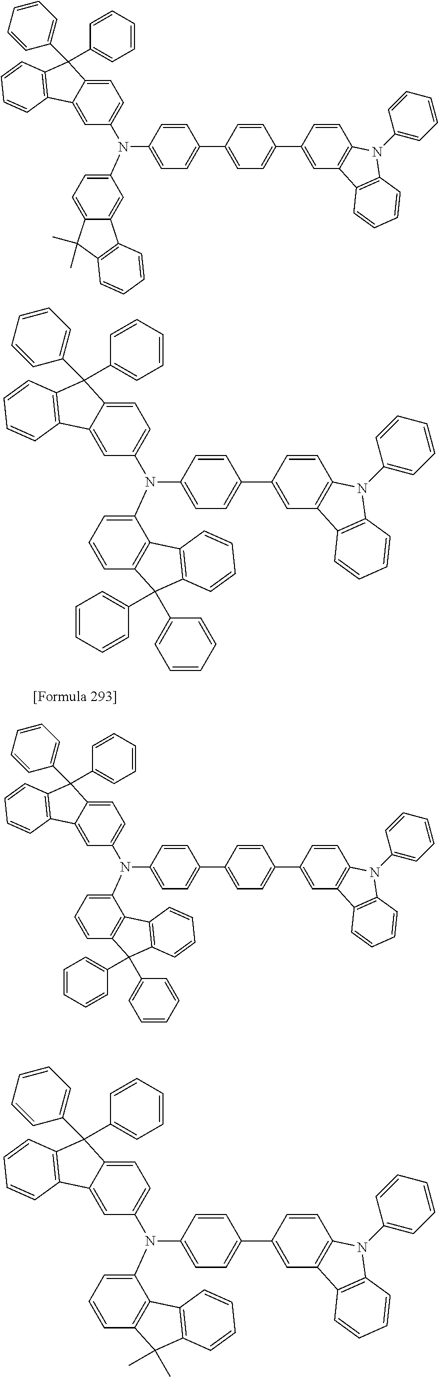

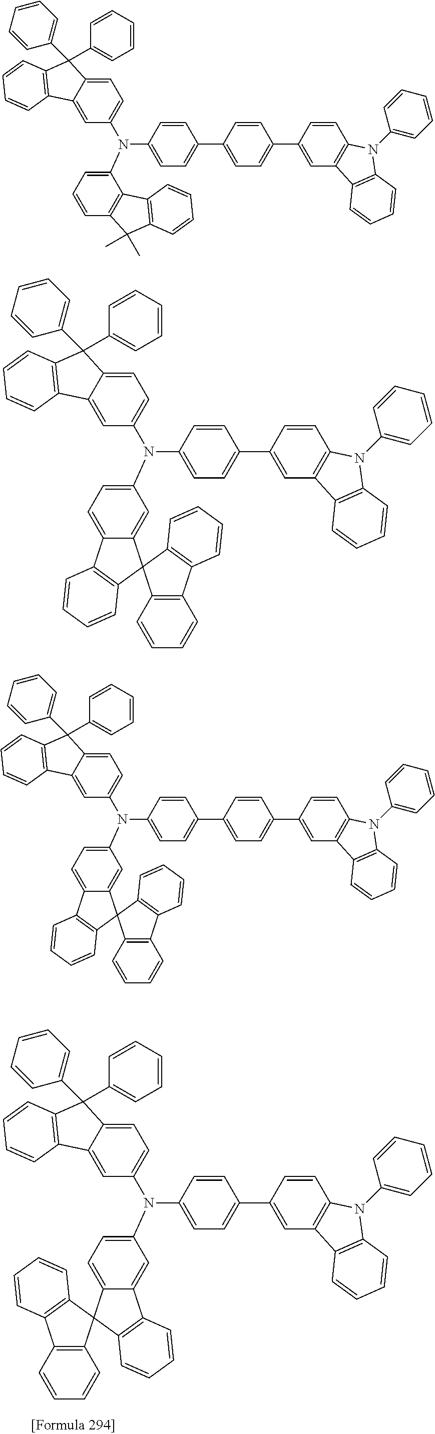

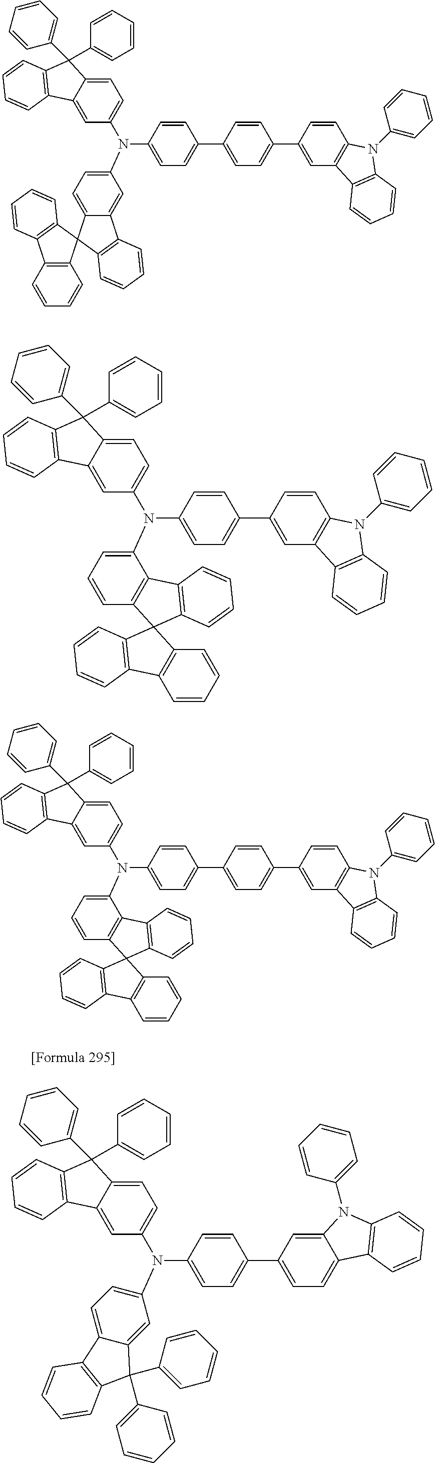

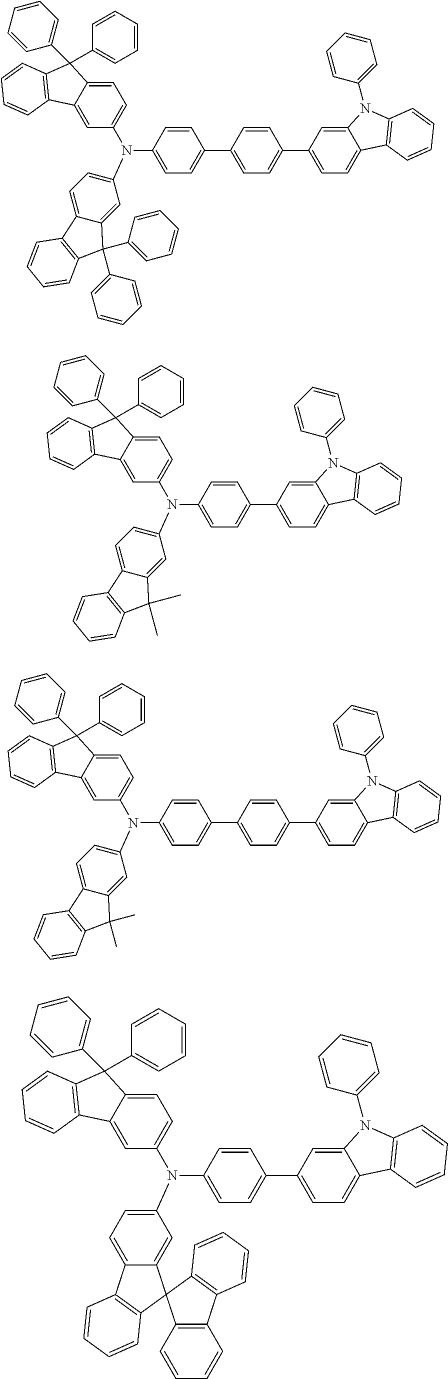

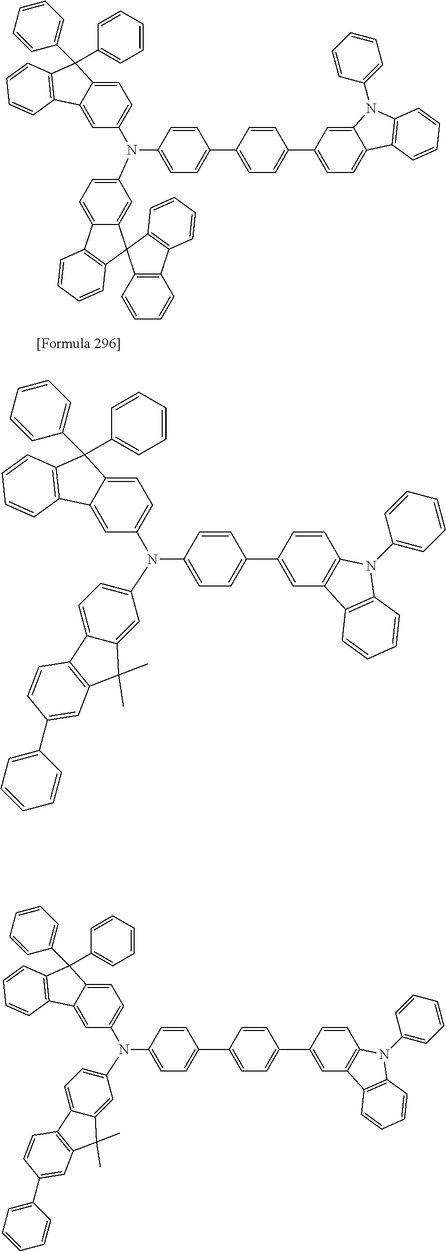 [SMILES: c1ccc(-n2c3ccccc3c3ccc(-c4ccc(-c5ccc(N(c6ccc7c(c6)-c6ccccc6C7(c6ccccc6)c6ccccc6)c6ccc7c(c6)C6(c8ccccc8-c8ccccc86)c6ccccc6-7)cc5)cc4)cc32)cc1 ; CC1(C)c2cc(-c3ccccc3)ccc2-c2ccc(N(c3ccc(-c4ccc5c(c4)c4ccccc4n5-c4ccccc4)cc3)c3ccc4c(c3)-c3ccccc3C4(c3ccccc3)c3ccccc3)cc21 ; CC1(C)c2cc(-c3ccccc3)ccc2-c2ccc(N(c3ccc(-c4ccc(-c5ccc6c(c5)c5ccccc5n6-c5ccccc5)cc4)cc3)c3ccc4c(c3)-c3ccccc3C4(c3ccccc3)c3ccccc3)cc21]

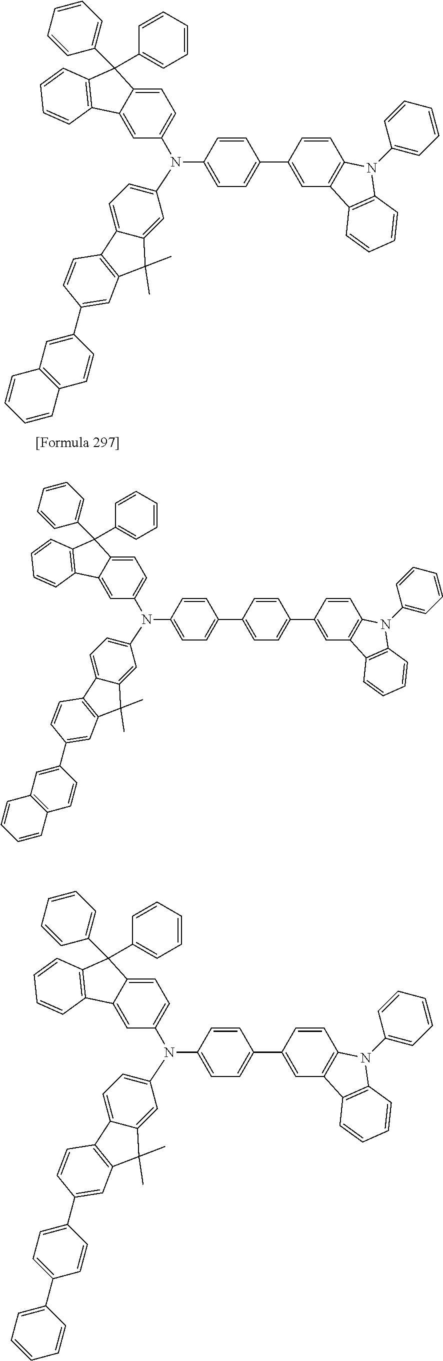 [SMILES: CC1(C)c2cc(-c3ccc4ccccc4c3)ccc2-c2ccc(N(c3ccc(-c4ccc5c(c4)c4ccccc4n5-c4ccccc4)cc3)c3ccc4c(c3)-c3ccccc3C4(c3ccccc3)c3ccccc3)cc21 ; CC1(C)c2cc(-c3ccc4ccccc4c3)ccc2-c2ccc(N(c3ccc(-c4ccc(-c5ccc6c(c5)c5ccccc5n6-c5ccccc5)cc4)cc3)c3ccc4c(c3)-c3ccccc3C4(c3ccccc3)c3ccccc3)cc21 ; CC1(C)c2cc(-c3ccc(-c4ccccc4)cc3)ccc2-c2ccc(N(c3ccc(-c4ccc5c(c4)c4ccccc4n5-c4ccccc4)cc3)c3ccc4c(c3)-c3ccccc3C4(c3ccccc3)c3ccccc3)cc21]

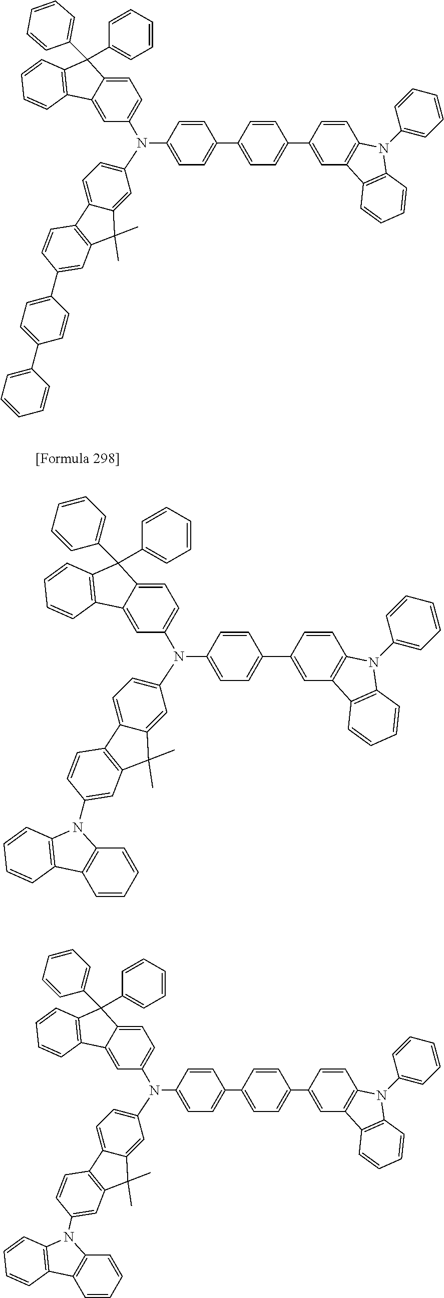 [SMILES: CC1(C)c2cc(-c3ccc(-c4ccccc4)cc3)ccc2-c2ccc(N(c3ccc(-c4ccc(-c5ccc6c(c5)c5ccccc5n6-c5ccccc5)cc4)cc3)c3ccc4c(c3)-c3ccccc3C4(c3ccccc3)c3ccccc3)cc21 ; CC1(C)c2cc(N(c3ccc(-c4ccc5c(c4)c4ccccc4n5-c4ccccc4)cc3)c3ccc4c(c3)-c3ccccc3C4(c3ccccc3)c3ccccc3)ccc2-c2ccc(-n3c4ccccc4c4ccccc43)cc21 ; CC1(C)c2cc(N(c3ccc(-c4ccc(-c5ccc6c(c5)c5ccccc5n6-c5ccccc5)cc4)cc3)c3ccc4c(c3)-c3ccccc3C4(c3ccccc3)c3ccccc3)ccc2-c2ccc(-n3c4ccccc4c4ccccc43)cc21]

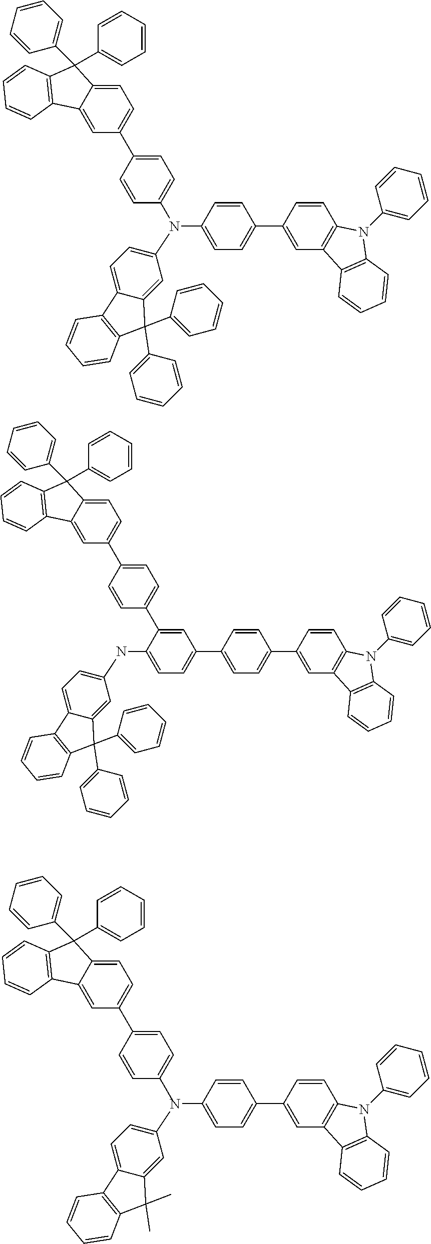

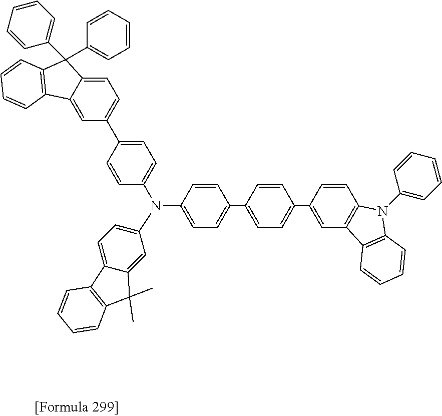 [SMILES: CC1(C)c2ccccc2-c2ccc(N(c3ccc(-c4ccc(-c5ccc6c(c5)c5ccccc5n6-c5ccccc5)cc4)cc3)c3ccc(-c4ccc5c(c4)-c4ccccc4C5(c4ccccc4)c4ccccc4)cc3)cc21]

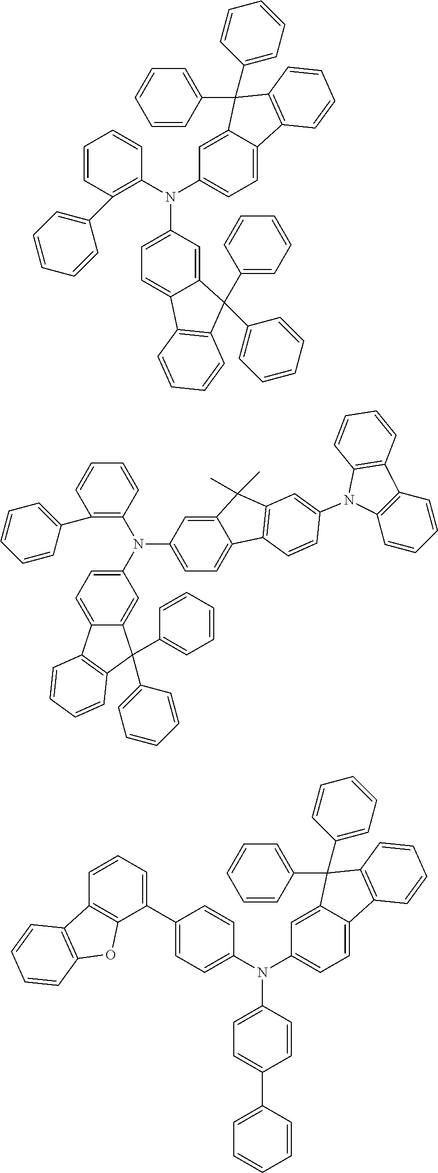 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; CC1(C)c2cc(N(c3ccc4c(c3)C(c3ccccc3)(c3ccccc3)c3ccccc3-4)c3ccccc3-c3ccccc3)ccc2-c2ccc(-n3c4ccccc4c4ccccc43)cc21 ; c1ccc(-c2ccc(N(c3ccc(-c4cccc5c4oc4ccccc45)cc3)c3ccc4c(c3)C(c3ccccc3)(c3ccccc3)c3ccccc3-4)cc2)cc1]

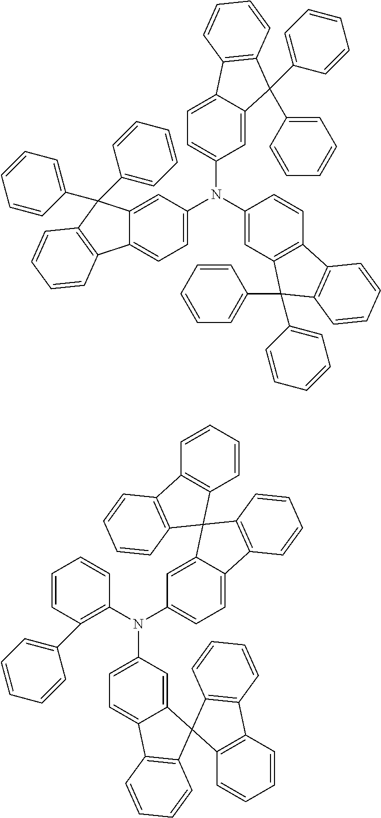 [SMILES: c1ccc(C2(c3ccccc3)c3ccccc3-c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc32)cc1 ; c1ccc(-c2ccccc2N(c2ccc3c(c2)C2(c4ccccc4-c4ccccc42)c2ccccc2-3)c2ccc3c(c2)C2(c4ccccc4-c4ccccc42)c2ccccc2-3)cc1]

The content of the third compound in the hole transporting layer 6 preferably ranges from 30 mol % to 100 mol % with respect to all components of the hole transporting layer 6, more preferably from 50 mol % to 100 mol %, further preferably from 80 mol % to 100 mol %. A possible compound contained in the hole transporting layer 6 along with the third compound is preferably a later-described hole-transporting compound.

The hole transporting layer 6 particularly preferably contains the third compound at substantially 100 mol %. The term “substantially” means that the hole transporting layer 6 consists of the third compound or additionally contains, for instance, a slight amount of inevitable impurities or the like coming from a material for forming the hole transporting layer 6.

### Substrate

The substrate 2 is used as a support for the organic EL device 1. For instance, glass, quartz, plastics and the like are usable as the substrate 2. A flexible substrate is also usable. The flexible substrate means a substrate that is bendable (flexible). The flexible substrate is exemplified by a plastic substrate made of, for instance, polycarbonate, polyarylate, polyethersulfone, polypropylene, polyester, polyvinyl fluoride, polyvinyl chloride, polyimide, or polyethylene naphthalate. Moreover, an inorganic vapor deposition film is also usable as the substrate 2.

### Anode

Metal having a large work function (specifically, 4.0 eV or more), alloy, an electrically conductive compound and a mixture thereof are preferably usable as the anode 3 formed on the substrate 2. Specific examples of the material for the anode include indium tin oxide (ITO), indium tin oxide containing silicon or silicon oxide, indium zinc oxide, tungsten oxide, indium oxide containing zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), nitrides of a metal material (e.g., titanium nitride) and the like are usable.

The above materials are typically formed into a film by sputtering. For instance, a target of the indium zinc oxide which is prepared by adding zinc oxide in a range from 1 mass % to 10 mass % relative to indium oxide is used for forming a film by sputtering. Moreover, for instance, as for the indium oxide containing tungsten oxide and zinc oxide, a target thereof prepared by adding tungsten oxide in a range from 0.5 mass % to 5 mass % and zinc oxide in a range from 0.1 mass % to 1 mass % relative to indium oxide is usable for forming a film by sputtering. In addition, vapor deposition, coating, ink jet printing, spin coating and the like may be used to form the anode 3.

Among the organic layers formed on the anode 3, since the hole injecting layer 5 formed adjacent to the anode 3 is formed of a composite material in which holes are easily injectable irrespective of the work function of the anode 3, other materials usable as an electrode material (e.g., a metal, alloy, electrically conductive compound, mixture thereof, and elements belonging to Group 1 or 2 in the periodic table of the elements) are also usable for the anode 3.

A material with a small work function such as elements belonging to Group 1 or 2 in the periodic table of the elements is also usable for the anode 3. For instance, alkali metals such as lithium (Li) and cesium (Cs), alkaline earth metals such as magnesium (Mg), calcium (Ca) and strontium (Sr), alloys (e.g., MgAg and AlLi) containing at least one of the alkali metal(s) and the alkaline earth metal(s), rare earth metals such as europium (Eu) and ytterbium (Yb), and alloys containing the rare earth metal(s) are also usable for the anode 3. When the anode 3 is formed of the alkali metal, alkaline earth metal and/or alloy thereof, vapor deposition and sputtering are usable. Further, when the anode 3 is formed of silver paste or the like, coating, ink jet printing and the like are usable.

### Hole Injecting Layer

The hole injecting layer 5 is a layer containing a highly hole-injectable substance. Examples of the highly hole-injectable substance include molybdenum oxide, titanium oxide, vanadium oxide, rhenium oxide, ruthenium oxide, chromium oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

Moreover, a high-molecule compound (e.g., an oligomer, dendrimer and polymer) is also usable as the highly hole-injectable substance. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl) methacrylamido] (abbreviation: PTPDMA), and poly[N,N′-bis(4-butylphenyl)-N,N′-bis(phenyl)benzidine] (abbreviation: Poly-TPD). Moreover, the examples of the high-molecule compound include a high-molecule compound added with an acid such as poly(3,4-ethylene dioxythiophene)/poly(styrene sulfonic acid) (PEDOT/PSS), and polyaniline/poly(styrene sulfonic acid) (PAni/PSS).

### Electron Transporting Layer

The electron transporting layer 8 is a layer containing a highly electron-transporting substance. For the electron transporting layer 8, 1) metal complexes such as an aluminum complex, beryllium complex and zinc complex, 2) heteroaromatic compounds such as an imidazole derivative, benzimidazole derivative, azine derivative, carbazole derivative, and phenanthroline derivative, and 3) high-molecule compounds are usable. Specifically, as a low-molecule organic compound, a metal complex such as Alq, tris(4-methyl-8-quinolinolato)aluminum (abbreviation: Almq₃), bis(10-hydroxybenzo[h]quinolinato)beryllium (abbreviation: BeBq₂), BAlq, Znq, ZnPBO and ZnBTZ are usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) are usable. In the first exemplary embodiment, a benzimidazole compound is suitably usable. The above-described substances mostly have an electron mobility of 10⁻⁶ cm²/(V·s) or more. However, any substance capable of an electron transporting performance higher than a hole transporting performance may be used for the electron transporting layer 8 instead of the above substances. The electron transporting layer 8 may be provided in the form of a single layer or a laminate of two or more layers made of the above substance(s).

Moreover, a high-molecule compound is also usable for the electron transporting layer 8. For instance, poly[(9,9-dihexylfluorene-2,7-diyl)-co-(pyridine-3,5-diyl)] (abbreviation: PF-Py) and poly[(9,9-dioctylfluorene-2,7-diyl)-co-(2,2′-bipyridine-6,6′-diyl)](abbreviation: PF-BPy) are usable.

### Electron Injecting Layer

The electron injecting layer 9 is a layer containing a highly electron-injectable substance. Examples of a material for the electron injecting layer 9 include alkali metal, alkaline earth metal and compound thereof, examples of which include lithium (Li), cesium (Cs), calcium (Ca), lithium fluoride (LiF), cesium fluoride (CsF), calcium fluoride (CaF₂), and lithium oxide (LiOx). In addition, a substance containing alkali metal, alkaline earth metal or compound thereof in the electron-transporting substance, i.e., a substance containing magnesium (Mg) in Alq, may be used. In this case, electrons can be more efficiently injected from the cathode 4. Alternatively, a composite material provided by mixing an organic compound with an electron donor may be used for the electron injecting layer 9. The composite material exhibits excellent electron injecting performance and electron transporting performance since the electron donor generates electron in the organic compound. In this arrangement, the organic compound is preferably a material exhibiting an excellent transforming performance of the generated electrons. Specifically, for instance, the above-described substance for the electron transporting layer 8 (e.g., the metal complex and heteroaromatic compound) is usable. The electron donor may be any substance exhibiting an electron donating performance to the organic compound. Specifically, an alkali metal, an alkaline earth metal or a rare earth metal is preferable, and examples of these metals include lithium, cesium, magnesium, calcium, erbium and ytterbium. Moreover, an alkali metal oxide and alkaline earth metal oxide are preferable, and examples of these oxides include lithium oxide, calcium oxide, and barium oxide. Further, Lewis base such as magnesium oxide is also usable. Furthermore, tetrathiafulvalene (abbreviation: TTF) is also usable.

### Cathode

Metal, alloy, an electrically conductive compound, a mixture thereof and the like, which have a small work function (specifically, 3.8 eV or less), are preferably usable as a material for the cathode 4. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

When the cathode 4 is formed of the alkali metal, alkaline earth metal and alloy thereof, vapor deposition and sputtering are usable. Further, when the cathode 4 is formed of silver paste or the like, coating, ink jet printing and the like are usable.

By providing the electron injecting layer 9, various conductive materials such as Al, Ag, ITO, graphene, and indium tin oxide containing silicon or silicon oxide are usable for forming the cathode 4 irrespective of the magnitude of the work function. The conductive materials can be formed into a film by sputtering, ink jet printing, spin coating and the like.

### Layer Formation Method(s)

A method for forming each layer of the organic EL device in the first exemplary embodiment is not limited except for the above particular description. Known methods of dry film-forming and wet film-forming are usable. Examples of the dry film-forming include vacuum deposition, sputtering, plasma deposition method and ion plating. Examples of the wet film-forming include spin coating, dipping, flow coating and ink-jet.

### Film Thickness

A film thickness of each of the organic layers in the organic EL device 1 according to the first exemplary embodiment is not limited except for the above particular description. The film thickness generally preferably ranges from several nanometers to 1 m, since too small thickness possibly causes defects such as a pin hole while too large thickness requires high voltage to be applied and lowers efficiency.

### Manufacturing Method of Compound of First Exemplary Embodiment

The compound according to the first exemplary embodiment can be manufactured by, for instance, a typically known method. The compound of the first exemplary embodiment can be synthesized by application of known substitution reactions and/or materials depending on a target compound according to the typically known method.

A hydrogen atom herein includes isotope having different numbers of neutrons, specifically, protium, deuterium and tritium.

The wording “carbon atoms forming a ring (ring carbon atoms)” herein means carbon atoms forming a saturated ring, unsaturated ring, or aromatic ring.

The number of carbon atoms forming a ring (also referred to as ring carbon atoms) herein means the number of carbon atoms included in atoms forming the ring itself of a compound in which the atoms are bonded to form the ring (e.g., a monocyclic compound, a fused ring compound, a cross-linked compound, a carbocyclic compound, and a heterocyclic compound). When the ring is substituted by a substituent, the “ring carbon atoms” do not include carbon(s) contained in the substituent. Unless specifically described, the same applies to the “ring carbon atoms” described later. For instance, a benzene ring has 6 ring carbon atoms, a naphthalene ring has 10 ring carbon atoms, a pyridinyl group has 5 ring carbon atoms, and a furanyl group has 4 ring carbon atoms. When the benzene ring and/or the naphthalene ring is substituted by, for instance, an alkyl group, the number of carbon atoms of the alkyl group is not included in the number of the ring carbon atoms. When a fluorene ring is substituted by, for instance, a fluorene ring (e.g., a spirofluorene ring), the number of carbon atoms of the fluorene ring as a substituent is not counted in the number of the ring carbon atoms for the fluorene ring.

“Atoms forming a ring (ring atoms)” herein means carbon atoms and hetero atoms forming a hetero ring including a saturated ring, unsaturated ring, or aromatic ring.

The number of atoms forming a ring (also referred to as ring atoms) herein means the number of atoms forming the ring itself of a compound in which the atoms are bonded to form the ring (e.g., a monocyclic compound, a fused ring compound, a cross-linked compound, a carbocyclic compound, and a heterocyclic compound). Atom(s) not forming the ring (e.g., a hydrogen atom for terminating the atoms forming the ring) and atoms included in a substituent substituting the ring are not counted in the number of the ring atoms. Unless specifically described, the same applies to the “ring atoms” described later. For instance, a pyridine ring has 6 ring atoms, a quinazoline ring has 10 ring atoms, and a furan ring has 5 ring atoms. Hydrogen atoms respectively bonded to the pyridine ring and the quinazoline ring and atoms forming the substituents are not counted in the number of the ring atoms. When a fluorene ring is substituted by, for instance, a fluorene ring (e.g., a spirofluorene ring), the number of atoms of the fluorene ring as a substituent is not included in the number of the ring atoms for the fluorene ring.

Next, each of substituents described in the above formulae will be described.

Examples of the aromatic hydrocarbon group (occasionally, referred to as aryl group) having 6 to 30 ring carbon atoms in the first exemplary embodiment include a phenyl group, biphenyl group, terphenyl group, naphthyl group, anthryl group, phenanthryl group, fluorenyl group, pyrenyl group, chrysenyl group, fluoranthenyl group, benz[a]anthryl group, benzo[c]phenanthryl group, triphenylenyl group, benzo[k]fluoranthenyl group, benzo[g]chrysenyl group, benzo[b]triphenylenyl group, picenyl group, and perylenyl group.

The aryl group in the first exemplary embodiment group preferably has 6 to 20 ring carbon atoms, more preferably 6 to 14 ring carbon atoms, further preferably 6 to 12 ring carbon atoms. Among such aryl groups, a phenyl group, biphenyl group, naphthyl group, phenanthryl group, terphenyl group, and fluorenyl group are particularly preferable. A carbon atom in a position 9 of each of 1-fluorenyl group, 2-fluorenyl group, 3-fluorenyl group and 4-fluorenyl group is preferably substituted by a later-described substituted or unsubstituted alkyl group having 1 to 30 carbon atoms or substituted or unsubstituted aryl group having 6 to 18 ring carbon atoms in the first exemplary embodiment.

The heterocyclic group (occasionally, referred to as heteroaryl group, heteroaromatic cyclic group, or aromatic heterocyclic group) having 5 to 30 ring atoms in the first exemplary embodiment preferably contains as a hetero atom at least one selected from the group consisting of nitrogen, sulfur, oxygen, silicon, selenium atom and germanium atom, more preferably at least one selected from the group consisting of nitrogen, sulfur and oxygen. Examples of the heteroaryl group having 5 to 30 ring atoms in the first exemplary embodiment include a pyridyl group, pyrimidinyl group, pyrazinyl group, pyridazynyl group, triazinyl group, quinolyl group, isoquinolinyl group, naphthyridinyl group, phthalazinyl group, quinoxalinyl group, quinazolinyl group, phenanthridinyl group, acridinyl group, phenanthrolinyl group, pyrrolyl group, imidazolyl group, pyrazolyl group, triazolyl group, tetrazolyl group, indolyl group, benzimidazolyl group, indazolyl group, imidazopyridinyl group, benzotriazolyl group, carbazolyl group, furyl group, thienyl group, oxazolyl group, thiazolyl group, isoxazolyl group, isothiazolyl group, oxadiazolyl group, thiadiazolyl group, benzofuranyl group, benzothiophenyl group, benzoxazolyl group, benzothiazolyl group, benzisoxazolyl group, benzisothiazolyl group, benzoxadiazolyl group, benzothiadiazolyl group, dibenzofuranyl group, dibenzothiophenyl group, piperidinyl group, pyrrolidinyl group, piperazinyl group, morpholyl group, phenazinyl group, phenothiazinyl group, and phenoxazinyl group.

The heteroaryl group in the first exemplary embodiment preferably has 5 to 20 ring atoms, more preferably 5 to 14 ring atoms. Among the above heterocyclic groups, a 1-dibenzofuranyl group, 2-dibenzofuranyl group, 3-dibenzofuranyl group, 4-dibenzofuranyl group, 1-dibenzothiophenyl group, 2-dibenzothiophenyl group, 3-dibenzothiophenyl group, 4-dibenzothiophenyl group, 1-carbazolyl group, 2-carbazolyl group, 3-carbazolyl group, 4-carbazolyl group and 9-carbazolyl group are particularly preferable. In 1-carbazolyl group, 2-carbazolyl group, 3-carbazolyl group and 4-carbazolyl group, the hydrogen atom bonded to the nitrogen atom in position 9 is preferably substituted by the substituted or unsubstituted aryl group having 6 to 30 ring carbon atoms or the substituted or unsubstituted heterocyclic group having 5 to 30 ring atoms according to the first exemplary embodiment.

In the first exemplary embodiment, the heteroaryl group may be a group derived from any one of moieties represented by formulae (XY-1) to (XY-18) below.

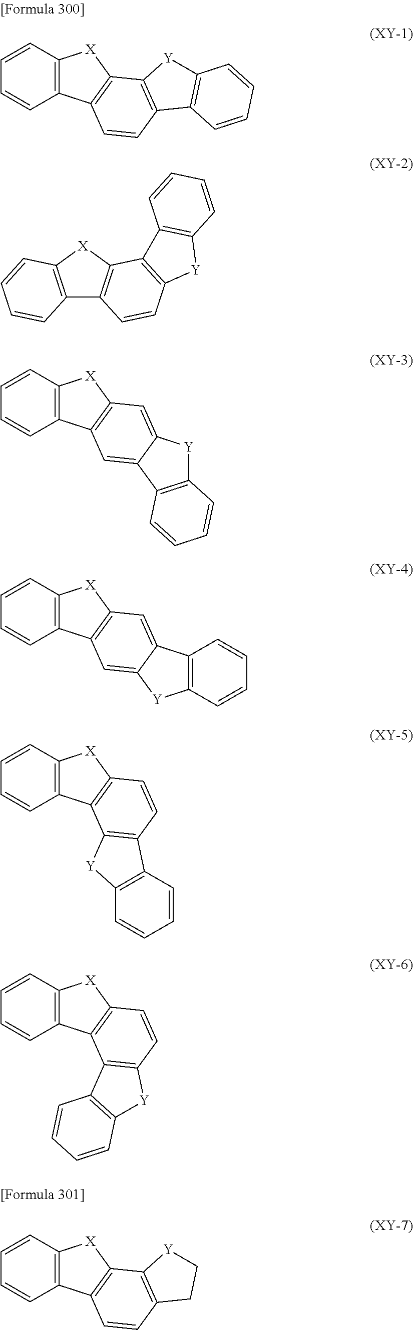

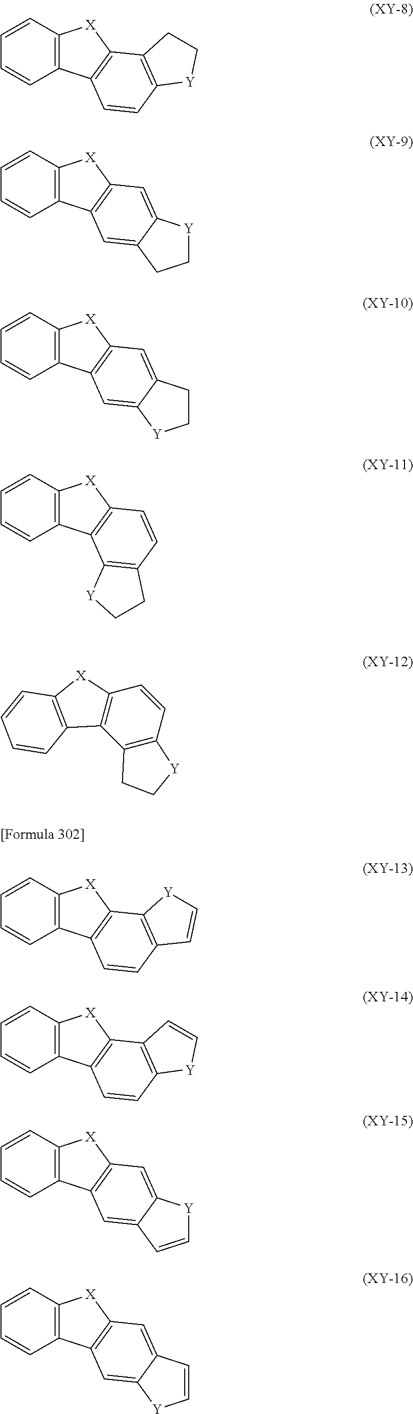

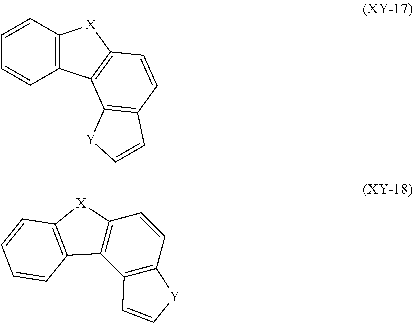

In the formulae (XY-1) to (XY-18), X and Y are each independently a hetero atom and preferably a nitrogen atom, oxygen atom, sulfur atom, selenium atom, silicon atom or germanium atom. The moieties represented by the formulae (XY-1) to (XY-18) have a bond in a predetermined position to be a heteroaryl group, which may be substituted or unsubstituted.

The substituted or unsubstituted carbazolyl group in the first exemplary embodiment may also be a group in which a ring is further fused to any one of carbazole rings represented by formulae below. Such a group may be substituted or unsubstituted. Further, the position of a bond may be changed as desired.

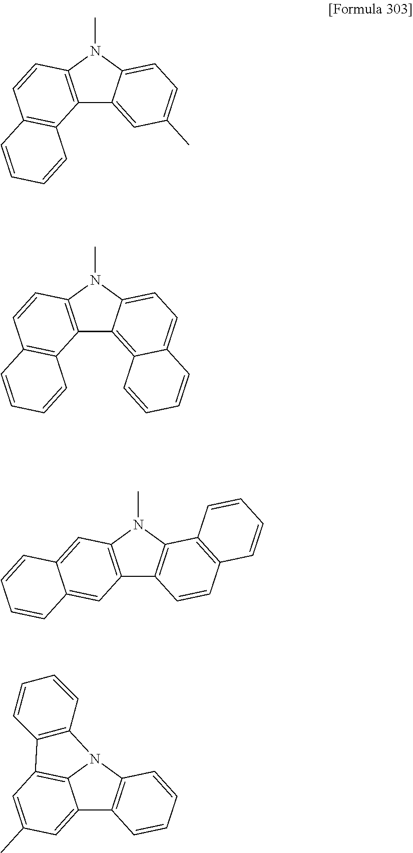

The alkyl group having 1 to 30 carbon atoms in the first exemplary embodiment may be linear, branched or cyclic. Examples of the linear or branched alkyl group include a methyl group, ethyl group, propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neo-pentyl group, amyl group, isoamyl group, 1-methylpentyl group, 2-methylpentyl group, 1-pentylhexyl group, 1-butylpentyl group, 1-heptyloctyl group and 3-methylpentyl group.

The linear or branched alkyl group in the exemplary embodiment preferably has 1 to 10 carbon atoms, more preferably 1 to 6 carbon atoms. Among the linear or branched alkyl group, a methyl group, ethyl group, propyl group, isopropyl group, n-butyl group, s-butyl group, isobutyl group, t-butyl group, n-pentyl group, n-hexyl group, amyl group, isoamyl group and neopentyl group are particularly preferable.

Examples of the cycloalkyl group having 3 to 30 carbon atoms in the exemplary embodiment are a cyclopropyl group, cyclobutyl group, cyclopentyl group, cyclohexyl group, 4-metylcyclohexyl group, adamantyl group and norbornyl group. The cycloalkyl group preferably has 3 to 10 ring carbon atoms, more preferably 5 to 8 ring carbon atoms. Among the cycloalkyl group, a cyclopentyl group and a cyclohexyl group are particularly preferable.

A halogenated alkyl group having 1 to 30 carbon atoms in the first exemplary embodiment may be a group in which hydrogen atom(s) of the above alkyl group having 1 to 30 carbon atoms is substituted by at least one halogen atom. Specific examples of the halogenated alkyl group include a fluoromethyl group, difluoromethyl group, trifluoromethyl group, fluoroethyl group, trifluoromethylmethyl group, trifluoroethyl group, and pentafluoroethyl group.

Examples of the halogen atom include a fluorine atom, chlorine atom, bromine atom and iodine atom, among which a fluorine atom is preferable.

Examples of a substituted amino group include an alkylamino group having 2 to 30 carbon atoms and an arylamino group having 6 to 60 ring carbon atoms.

The alkylamino group having 2 to 30 carbon atoms is represented by —NHR_{V} or —N(R_{V})₂. R_{V} is exemplified by the alkyl group having 1 to 30 carbon atoms.

The arylamino group having 6 to 60 ring carbon atoms is represented by —NHR_{W} or —N(R_{W})₂. R_{W} is exemplified by the above aryl group having 6 to 30 ring carbon atoms.

The alkoxy group having 1 to 30 carbon atoms is represented by —OZ₁. Z₁ is exemplified by the above alkyl group having 1 to 30 carbon atoms. Examples of the alkoxy group include a methoxy group, ethoxy group, propoxy group, butoxy group, pentyloxy group and hexyloxy group. The alkoxy group preferably has 1 to 20 carbon atoms.

A halogenated alkoxy group provided by substituting the alkoxy group with a halogen atom is exemplified by a halogenated alkoxy group provided by substituting the alkoxy group having 1 to 30 carbon atoms with one or more fluorine groups.

The aryloxy group having 6 to 30 ring carbon atoms is represented by —OZ₂. Z₂ is exemplified by the above aryl group having 6 to 30 ring carbon atoms. The aryloxy group preferably has 6 to 20 ring carbon atoms. The aryloxy group is exemplified by a phenoxy group.

The arylthio group having 6 to 30 ring carbon atoms is represented by —SR_{W}. R_{W} is exemplified by the above aryl group having 6 to 30 ring carbon atoms. The arylthio group preferably has 6 to 20 ring carbon atoms.

“Unsubstituted” in “substituted or unsubstituted” herein means that a group is not substituted by the above-described substituents but bonded to a hydrogen atom.

“XX to YY carbon atoms” in “substituted or unsubstituted ZZ group having XX to YY carbon atoms” herein means the number of the carbon atoms of an unsubstituted ZZ group and does not count the number of the carbon atoms of a substituent(s) of the substituted ZZ group. Herein, “YY” is larger than “XX.” “XX” and “YY” each mean an integer of 1 or more.

“XX to YY atoms” in “substituted or unsubstituted ZZ group having XX to YY atoms” herein means the number of atoms of an unsubstituted ZZ group and does not count the number of the atoms of a substituent(s) of the substituted ZZ group. Herein, “YY” is larger than “XX.” “XX” and “YY” each mean an integer of 1 or more.

Examples of the substituent meant by “substituted or unsubstituted” include an aromatic hydrocarbon group, heterocyclic group, alkyl group (linear or branched alkyl group, cycloalkyl group and halogenated alkyl group), cyano group, amino group, substituted amino group, halogen atom, alkoxy group, aryloxy group, arylthio group, aralkyl group, substituted phosphoryl group, substituted silyl group, nitro group, carboxy group, alkenyl group, alkynyl group, alkylthio group, alkylsilyl group, arylsilyl group, and hydroxyl group.

Among the above substituents meant by “substituted or unsubstituted”, an aromatic hydrocarbon group, heterocyclic group, alkyl group, halogen atom, alkylsilyl group, arylsilyl group and cyano group are preferable, and the above specific substituents of each of these substituents are more preferable.

These substituents meant by “substituted or unsubstituted” may be further substituted by one selected from the group consisting of an aromatic hydrocarbon group, heterocyclic group, alkyl group (linear or branched alkyl group, cycloalkyl group and halogenated alkyl group), substituted phosphoryl group, alkylsilyl group, arylsilyl group, alkoxy group, aryloxy group, alkylamino group, arylamino group, alkylthio group, arylthio group, alkenyl group, alkynyl group, aralkyl group, halogen atom, cyano group, hydroxyl group, nitro group, and carboxy group. In addition, plural ones of these substituents may be bonded together to form a ring.

The alkenyl group is preferably an alkenyl group having 2 to 30 carbon atoms that may be linear, branched or cyclic. Examples of such an alkenyl group include a vinyl group, propenyl group, butenyl group, oleyl group, eicosapentaenyl group, docosahexaenyl group, styryl group, 2,2-diphenylvinyl group, 1,2,2-triphenylvinyl group, 2-phenyl-2-propenyl group, cyclopentadienyl group, cyclopentenyl group, cyclohexenyl group and cyclohexadienyl group.

The alkynyl group is preferably an alkynyl group having 2 to 30 carbon atoms that may be linear, branched or cyclic. Examples of such an alkynyl group include an ethynyl group, a propynyl group and a 2-phenylethynyl group.

The alkylthio group having 1 to 30 carbon atoms is represented by —SR_{V}. R_{V} is exemplified by the alkyl group having 1 to 30 carbon atoms. The alkylthio group preferably has 1 to 20 carbon atoms.

The substituted or unsubstituted aralkyl group having 7 to 30 carbon atoms is represented by —Z₃—Z₄. Z₃ is exemplified by an alkylene group derived from the above alkyl group having 1 to 30 carbon atoms. Z₄ is exemplified by the examples of the aryl group having 6 to 30 ring carbon atoms. In the aralkyl group having 7 to 30 carbon atoms, the aryl moiety represented by Z₄ preferably has 6 to 20 ring carbon atoms, more preferably 6 to 12, and the alkyl moiety represented by Z₃ preferably has 1 to 20 carbon atoms, more preferably 1 to 10, further preferably 1 to 6. Examples of the aralkyl group include a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

The substituted phosphoryl group is represented by a formula (P) below.

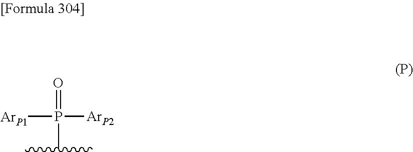

In the formula (P), Ar_{P1} and Ar_{P2} are each independently a substituent, preferably a substituent selected from the group consisting of an alkyl group having 1 to 30 carbon atoms and an aryl group having 6 to 30 ring carbon atoms, more preferably a substituent selected from the group consisting of an alkyl group having 1 to 10 carbon atoms and an aryl group having 6 to 20 ring carbon atoms, further preferably a substituent selected from the group consisting of an alkyl group having 1 to 6 carbon atoms and an aryl group having 6 to 14 ring carbon atoms.

Examples of the substituted silyl group include an alkylsilyl group having 3 to 30 carbon atoms and an arylsilyl group having 6 to 30 ring carbon atoms.

The alkylsilyl group having 3 to 30 carbon atoms in the first exemplary embodiment is exemplified by a trialkylsilyl group having the above examples of the alkyl group having 1 to 30 carbon atoms. Specific examples of the alkylsilyl group are a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyl dimethylsilyl group, propyldimethylsilyl group, and triisopropylsilyl group. The three alkyl groups in the trialkylsilyl group may be mutually the same or different.

Examples of the arylsilyl group having 6 to 30 ring carbon atoms in the first exemplary embodiment include a dialkylarylsilyl group, alkyldiarylsilyl group and triarylsilyl group.

The dialkylarylsilyl group is exemplified by a dialkylarylsilyl group having two of the examples of the alkyl group having 1 to 30 carbon atoms and one of the examples of the aryl group having 6 to 30 ring carbon atoms. The dialkylarylsilyl group preferably has 8 to 30 carbon atoms.

The alkyldiarylsilyl group is exemplified by an alkyldiarylsilyl group having one of the examples of the alkyl group having 1 to 30 carbon atoms and two of the examples of the aryl group having 6 to 30 ring carbon atoms. The dialkylarylsilyl group preferably has 13 to 30 carbon atoms.

The triarylsilyl group is exemplified by a triarylsilyl group having three of the examples of the aryl group having 6 to 30 ring carbon atoms. The triarylsilyl group preferably has 18 to 30 carbon atoms.

Examples of the aromatic hydrocarbon group and the heterocyclic group as the linking groups herein include divalent or multivalent groups obtained by removing one or more atoms from the above monovalent aromatic hydrocarbon group and heterocyclic group.

When the substituents are bonded to each other to form a cyclic structure, the cyclic structure herein is a saturated ring, an unsaturated ring, an aromatic hydrocarbon ring or a hetero ring. In addition, the cyclic structure formed by the bonded substituents may be substituted or unsubstituted.

Examples of the aromatic hydrocarbon ring and the hetero ring herein include a cyclic structure from which the above monovalent group is derived.

Electronic Device

The organic EL device 1 according to the first exemplary embodiment is usable in electronic devices such as a display unit and a light-emitting unit. Examples of the display unit include display components such as an organic EL panel module, TV, mobile phone, tablet, and personal computer. Examples of the light-emitting unit include an illuminator and a vehicle light.

In the first exemplary embodiment, the emitting layer 7 contains the first compound represented by the formula (1) and the fluorescent second compound, and the hole transporting layer 6 contains the third compound represented by the formula (3). Such an arrangement enables the organic EL device 1 to exhibit an improved external quantum efficiency (EQE) with a reduced drive voltage.

Typically, an anthracene derivative having a molecular structure consisting of a hydrocarbon skeleton (such an anthracene derivative being hereinafter occasionally referred to as a hydrocarbon anthracene derivative) has been used as a host material for a fluorescent emitting layer.

The first compound is supposed to have a large electron mobility as compared with the hydrocarbon anthracene derivative. Thus, when the emitting layer contains the first compound, the drive voltage is expected to be lowered. On the other hand, injection of an insufficient amount of holes from the hole transporting layer into the emitting layer may cause collision between generated excitons and electrons in the emitting layer near the hole transporting layer. Such a collision between the excitons and the electrons would deactivate the excitons, resulting in a lowered luminous efficiency.

The hole transporting layer made of a hole transporting material having an ionization potential more suitable for the ionization potential of the first compound is supposed to facilitate injection of holes into the emitting layer. Accordingly, the first exemplary embodiment employs the compound represented by the formula (3) as the third compound to form the hole transporting layer. When the third compound is used to form the hole transporting layer, injection of holes into the emitting layer is supposed to be facilitated to prevent the deactivation of excitons. Consequently, the luminous efficiency of the organic EL device according to the first exemplary embodiment would be improved while the drive voltage of the organic EL device is still reduced with the assistance of the first compound contained in the emitting layer.

### Second Exemplary Embodiment

An arrangement of an organic EL device according to a second exemplary embodiment will be described. In the description of the second exemplary embodiment, the same components as those in the first exemplary embodiment are denoted by the same reference signs and names to simplify or omit an explanation of the components. In the second exemplary embodiment, any materials and compounds that are not specified may be the same as those in the first exemplary embodiment.

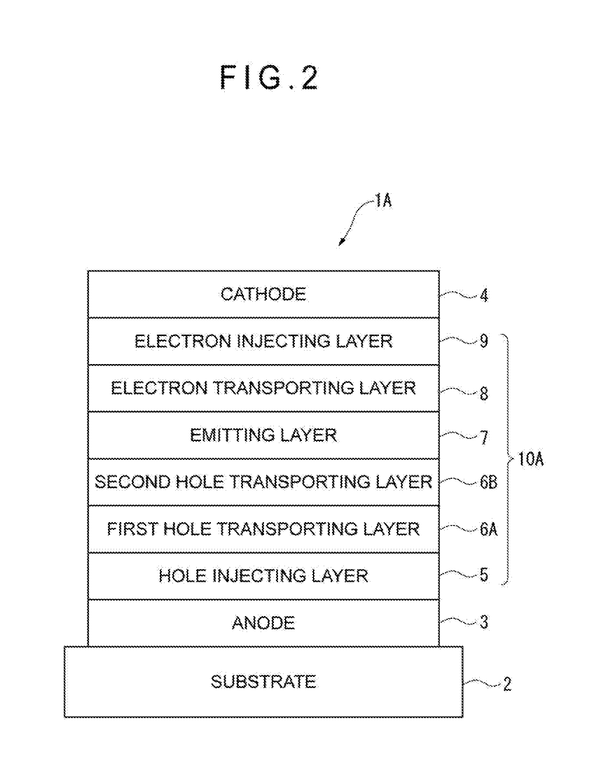

FIG. 2 schematically shows an arrangement of an exemplary organic EL device 1A according to the second exemplary embodiment.

The organic EL device 1A of the second exemplary embodiment is different from the organic EL device 1 of the first exemplary embodiment in terms of an arrangement of an organic layer. Specifically, an organic layer 10A of the organic EL device 1A includes laminated first hole transporting layer 6A and second hole transporting layer 6B, whereas the organic EL device 1 includes the hole transporting layer 6 in the form of a single layer.

The third compound is contained in at least either the first hole transporting layer 6A or the second hole transporting layer 6B. The third compound may be contained in either the first hole transporting layer 6A or the second hole transporting layer 6B or in both. When the third compound is contained in both the first hole transporting layer 6A and the second hole transporting layer 6B, the third compound in the first hole transporting layer 6A and the third compound in the second hole transporting layer 6B preferably have different structures in a range satisfying the structural conditions shown by the formula (3).

The hole transporting layer is a layer containing a highly hole-transporting substance. Either the first hole transporting layer 6A or the second hole transporting layer 6B also preferably contains a hole-transporting compound different from the third compound.

For instance, an aromatic amine compound, carbazole derivative and anthracene derivative are usable for the hole transporting layer. Specifically, for instance, an aromatic amine compound is usable for the hole transporting layer. Examples of the aromatic amine compound include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

A carbazole derivative and an anthracene derivative may be used for the hole transporting layer. Examples of the carbazole derivative include CBP, 9-[4-(N-carbazolyl)]phenyl-10-phenylanthracene (CzPA), and 9-phenyl-3-[4-(10-phenyl-9-anthryl)phenyl]-9H-carbazole (PCzPA). Examples of the anthracene derivative include t-BuDNA, DNA and DPAnth. High-molecular weight compounds such as poly(N-vinylcarbazole) (abbreviation: PVK) and poly(4-vinyltriphenylamine) (abbreviation: PVTPA) are also usable.

However, any substance having a hole transporting performance higher than an electron transporting performance may be used instead of the above substances.

When the hole transporting layer includes two or more layers, one of the layers with a larger energy gap is preferably provided closer to the emitting layer 7.

In the second exemplary embodiment, the emitting layer 7 contains the first compound represented by the formula (1) and the fluorescent second compound, and at least either the first hole transporting layer 6A or the second hole transporting layer 6B contains the third compound represented by the formula (3). Such an arrangement enables the organic EL device 1A to exhibit an improved external quantum efficiency (EQE) with a reduced drive voltage. The organic EL device 1A according to the second exemplary embodiment is usable in electronic devices such as a display unit and a light-emitting unit.

### Modification of Embodiments

It should be noted that the invention is not limited to the above exemplary embodiments but may include any modification and improvement compatible with the invention.

An arrangement of the organic EL device of the invention is not particularly limited to the arrangements described in the above exemplary embodiments.

For instance, a blocking layer may be provided adjacent to an anode-side or a cathode-side of the emitting layer. The blocking layer is preferably provided in contact with the emitting layer to at least block holes, electrons or excitons.

For instance, when the blocking layer is provided in contact with the cathode-side of the emitting layer, the blocking layer permits transport of electrons, but blocks holes from reaching a layer provided near the cathode (e.g., the electron transporting layer) beyond the blocking layer. When the organic EL device includes the electron transporting layer, the blocking layer is preferably interposed between the emitting layer and the electron transporting layer.

Further, the blocking layer may be provided in contact with the emitting layer to prevent an excitation energy from leaking from the emitting layer into neighboring layers. The blocking layer blocks excitons generated in the emitting layer from moving into a layer provided near the electrode (e.g., the electron transporting layer and the hole transporting layer) beyond the blocking layer.

The emitting layer is preferably in contact with the blocking layer. Further, specific arrangements and configurations for practicing the invention may be altered to other arrangements and configurations compatible with the invention.

### EXAMPLES

Examples of the invention will be described. However, the invention is not limited to Examples.

### Manufacture 1 of Organic EL Device

Compounds used for manufacturing organic EL devices are shown below.

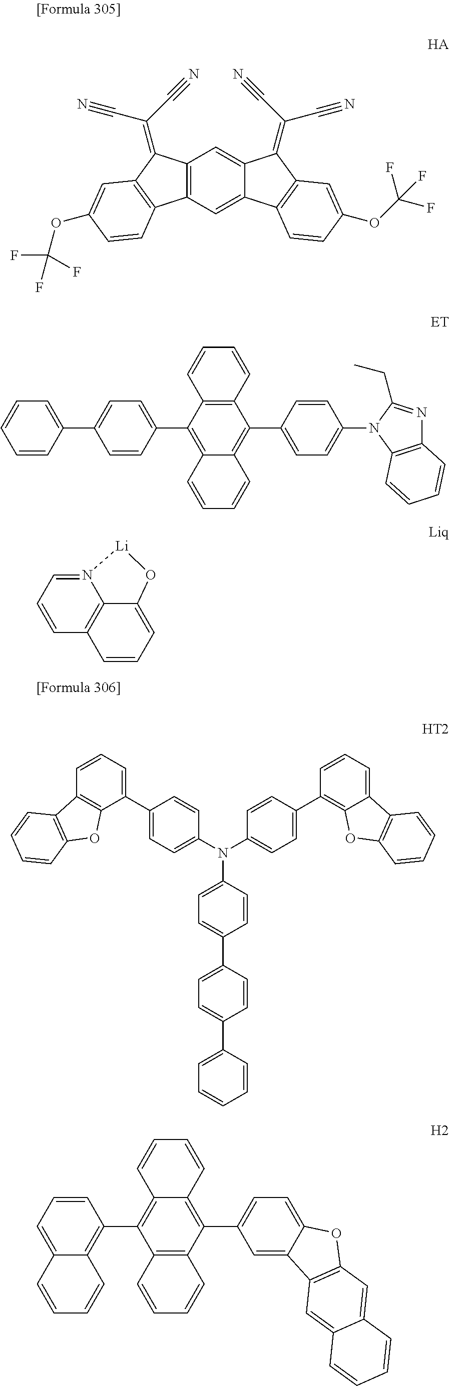

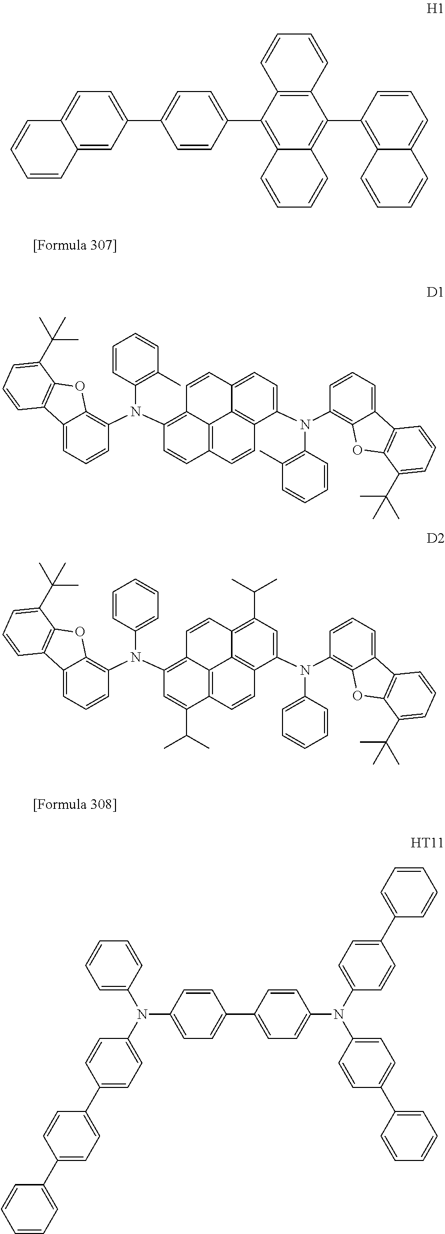

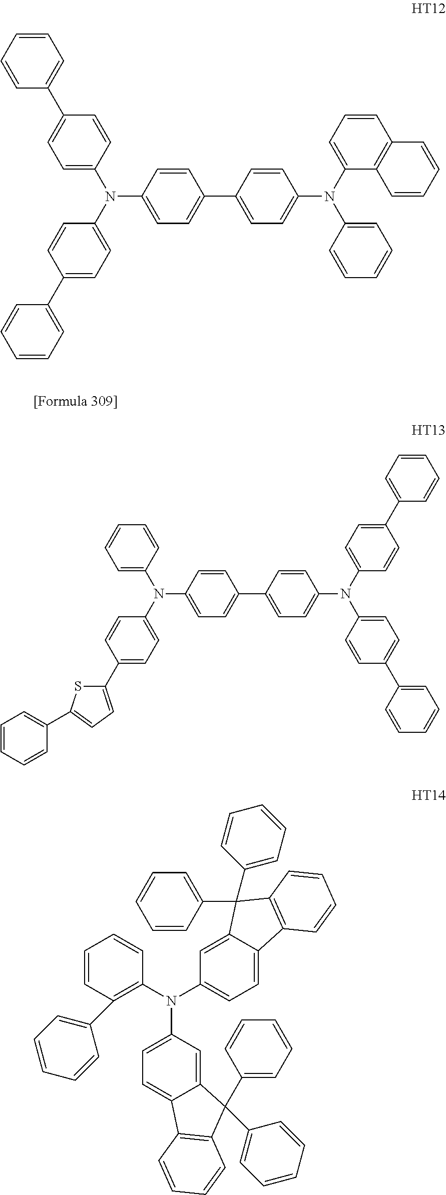 [SMILES: c1ccc(-c2ccc(N(c3ccc(-c4ccccc4)cc3)c3ccc(-c4ccc(N(c5ccccc5)c5cccc6ccccc56)cc4)cc3)cc2)cc1 ; c1ccc(-c2ccc(N(c3ccc(-c4ccccc4)cc3)c3ccc(-c4ccc(N(c5ccccc5)c5ccc(-c6ccc(-c7ccccc7)s6)cc5)cc4)cc3)cc2)cc1 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

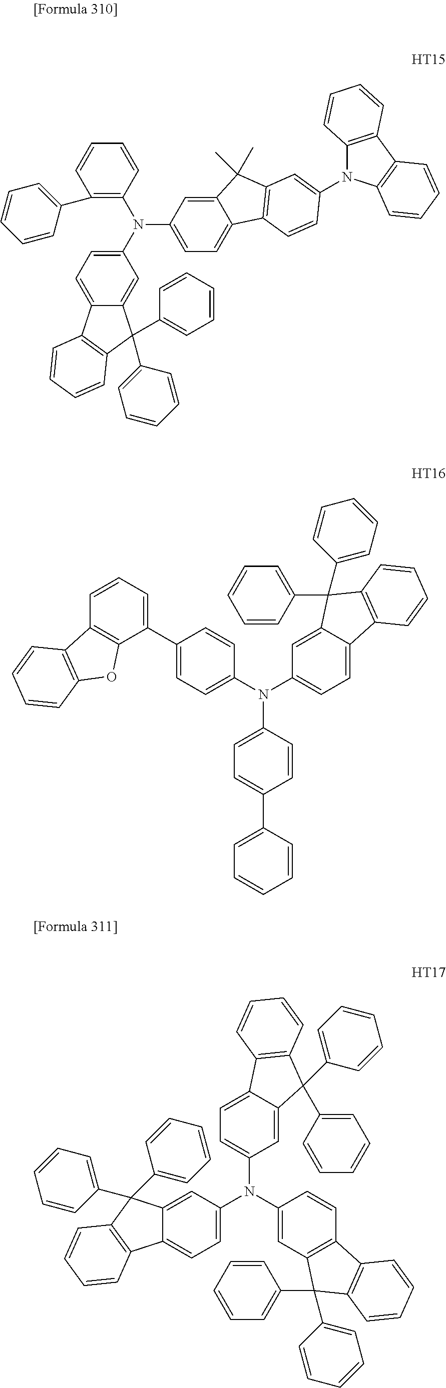 [SMILES: CC1(C)c2cc(N(c3ccc4c(c3)C(c3ccccc3)(c3ccccc3)c3ccccc3-4)c3ccccc3-c3ccccc3)ccc2-c2ccc(-n3c4ccccc4c4ccccc43)cc21 ; c1ccc(-c2ccc(N(c3ccc(-c4cccc5c4oc4ccccc45)cc3)c3ccc4c(c3)C(c3ccccc3)(c3ccccc3)c3ccccc3-4)cc2)cc1 ; c1ccc(C2(c3ccccc3)c3ccccc3-c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc32)cc1]

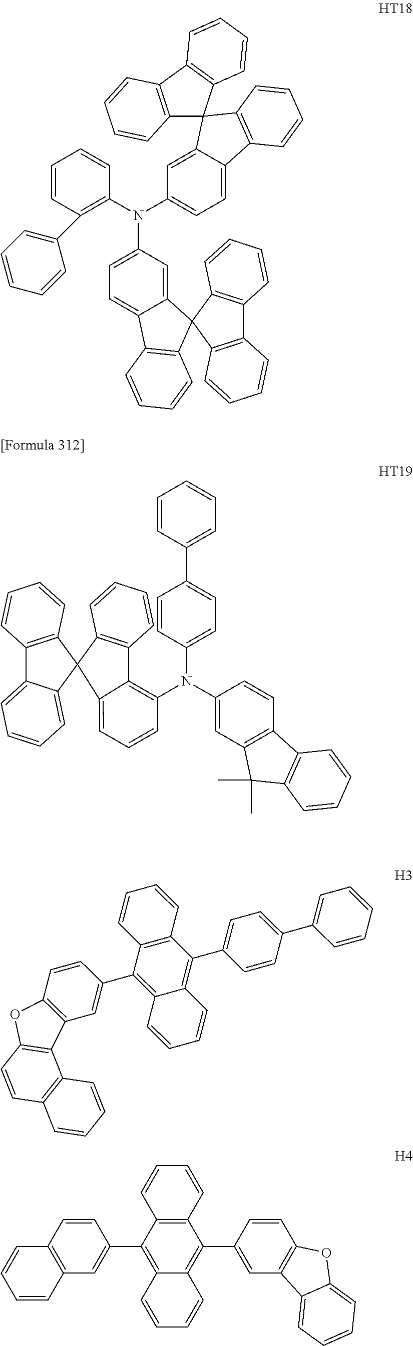

### Example 1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for 30 minutes. A film of ITO was set to be 130-nm thick.

After the glass substrate having the transparent electrode line was cleaned, the glass substrate was mounted on a substrate holder of a vacuum evaporation apparatus. Initially, the compound HA was deposited on a surface of the glass substrate where the transparent electrode line was provided in a manner to cover the transparent electrode, thereby forming a 5-nm thick HA film of the compound HA to form a hole injecting layer.

Next, on the hole injecting layer, the compound HT14 was deposited to form a 80-nm thick HT14 film, thereby providing a first hole transporting layer.

Next, on the first hole transporting layer, the compound HT2 was deposited to form a 10-nm thick HT2 film, thereby providing a second hole transporting layer.

Next, on the second hole transporting layer, the compound H2 and the blue-emitting fluorescent material D1 were co-deposited to form a 25-nm thick emitting layer. A concentration of the compound D1 in the emitting layer was 4 mass %.

Subsequent to the formation of the emitting layer, the compound ET and 8-quinolinolato lithium (Liq) were co-deposited to form a 25-nm thick electron transporting layer. A concentration of Liq in the electron transporting layer was 50 mass %.

Liq was deposited on the electron transporting layer to form a 1-nm thick electron injecting layer.

A metal Al was deposited on the electron injecting layer to form an 80-nm thick metal cathode.

Thus, an organic EL device of Example 1 was manufactured.

A device arrangement of the organic EL device of Example 1 is roughly shown as follows.

ITO(130)/HA(5)/HT14(80)/HT2(10)/H2:D1(25, 4%)/ET:Liq(25, 50%)/Liq(1)/Al(80)

Numerals in parentheses represent film thicknesses (unit: nm). The numerals represented by percentage in the same parentheses indicate a concentration (mass %) of the compound D1 in the emitting layer or a concentration (mass %) of Liq in the electron transporting layer.

### Example 2

An organic EL device of Example 2 was manufactured in the same manner as the organic EL device of Example 1 except that the compound HT15 was used in place of the compound HT14 in the first hole transporting layer of Example 1.

A device arrangement of the organic EL device of Example 2 is roughly shown as follows.

ITO(130)/HA(5)/HT15(80)/HT2(10)/H2:D1(25, 4%)/ET:Liq(25, 50%)/Liq(1)/Al(80)

### Comparative 1

An organic EL device of Comparative 1 was manufactured in the same manner as the organic EL device of Example 1 except that the compound HT11 was used in place of the compound HT14 in the first hole transporting layer of Example 1 and the compound H1 was used in place of the compound H2 in the emitting layer.

A device arrangement of the organic EL device of Comparative 1 is roughly shown as follows.

ITO(130)/HA(5)/HT11(80)/HT2(10)/H1:D1(25, 4%)/ET:Liq(25, 50%)/Liq(1)/Al(80)

### Comparative 2

An organic EL device of Comparative 2 was manufactured in the same manner as the organic EL device of Comparative 1 except that the compound H2 was used in place of the compound H1 in the emitting layer of Comparative 1.

A device arrangement of the organic EL device of Comparative 2 is roughly shown as follows.

ITO(130)/HA(5)/HT11(80)/HT2(10)/H2:D1(25, 4%)/ET:Liq(25, 50%)/Liq(1)/Al(80)

### Comparative 3

An organic EL device of Comparative 3 was manufactured in the same manner as the organic EL device of Comparative 2 except that the compound HT12 was used in place of the compound HT11 in the first hole transporting layer of Comparative 2.

A device arrangement of the organic EL device of Comparative 3 is roughly shown as follows.

ITO(130)/HA(5)/HT12(80)/HT2(10)/H2:D1(25, 4%)/ET:Liq(25, 50%)/Liq(1)/Al(80)

### Comparative 4

An organic EL device of Comparative 4 was manufactured in the same manner as the organic EL device of Comparative 2 except that the compound HT13 was used in place of the compound HT11 in the first hole transporting layer of Comparative 2.

A device arrangement of the organic EL device of Comparative 4 is roughly shown as follows.

ITO(130)/HA(5)/HT13(80)/HT2(10)/H2:D1(25, 4%)/ET:Liq(25, 50%)/Liq(1)/Al(80)

### Evaluation 1 of Organic EL Devices

The prepared organic EL devices of Examples 1 and 2 and Comparatives 1 to 4 were evaluated as below. The evaluation results are shown in Table 1.

### Drive Voltage

Voltage was applied between the ITO transparent electrode and the metal Al cathode such that a current density was 10 mA/cm², where the voltage (unit: V) was measured.

### External Quantum Efficiency (EQE)

Voltage was applied on each of the organic EL devices such that a current density was 10 mA/cm², where spectral radiance spectra were measured by a spectroradiometer (CS-1000 manufactured by Konica Minolta, Inc.). The external quantum efficiency (EQE) (unit: %) was calculated from each of the obtained spectral radiance spectra, assuming that the spectra were provided under a Lambertian radiation.

TABLE 1

Compound

| | First Hole | | | |
|---|---|---|---|---|
| | Transporting | Emitting | Voltage | EQE |
| | Layer | Layer | [V] | [%] |
| Ex. 1 | HT14 | H2 | 3.29 | 9.2 |
| Ex. 2 | HT15 | H2 | 3.20 | 10.0 |
| Comp. 1 | HT11 | H1 | 3.67 | 7.6 |
| Comp. 2 | HT11 | H2 | 3.37 | 7.2 |
| Comp. 3 | HT12 | H2 | 3.26 | 7.3 |
| Comp. 4 | HT13 | H2 | 3.25 | 6.9 |

In the organic EL device of Comparative 1, the compound H1 was used for the emitting layer. The compound H1, which is the hydrocarbon anthracene derivative, has no naphthobenzofuran skeleton in the molecule unlike the compound H2. This would be why the organic EL device of Comparative 1 required a higher drive voltage than those of Examples 1 and 2 and Comparatives 2 to 4.

The organic EL device of Comparative 2, which used the compound H2 in place of the compound H1, required a lower drive voltage than that of Comparative 1. However, the external quantum efficiency of Comparative 2 was also lower than that of Comparative 1.

Accordingly, the organic EL devices of Comparatives 3 and 4 respectively used the compound HT12 and the compound HT13 as the compound for the first hole transporting layer instead. However, the resulting external quantum efficiencies were lowered to a level comparable to or less than that of Comparative 2.

In contrast, the organic EL devices of Examples 1 and 2 each required a lower drive voltage and exhibited a higher external quantum efficiency (EQE) than the organic EL devices of Comparatives 1 to 4. The organic EL devices of Examples 1 and 2 each included the emitting layer containing the first compound represented by the formula (1) and the blue-emitting fluorescent second compound, and the first hole transporting layer containing the third compound represented by the formula (3). Such an arrangement is supposed to improve the performance of the organic EL devices.

### Manufacture 2 of Organic EL Device

### Example 3

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for 30 minutes. A film of ITO was set to be 130-nm thick.

After the glass substrate having the transparent electrode line was cleaned, the glass substrate was mounted on a substrate holder of a vacuum evaporation apparatus. Initially, the compound HA was deposited on a surface of the glass substrate where the transparent electrode line was provided in a manner to cover the transparent electrode, thereby forming a 5-nm thick HA film of the compound HA to form a hole injecting layer.

Next, on the hole injecting layer, the compound HT16 was deposited to form a 105-nm thick HT16 film, thereby providing a first hole transporting layer.

Next, on the first hole transporting layer, the compound HT2 was deposited to form a 15-nm thick HT2 film, thereby providing a second hole transporting layer.

Next, on the second hole transporting layer, the compound H2 and the blue-emitting fluorescent material D2 were co-deposited to form a 20-nm thick emitting layer. A concentration of the compound D2 in the emitting layer was 4 mass %.

Subsequent to the formation of the emitting layer, the compound ET and 8-quinolinolato lithium (Liq) were co-deposited to form a 30-nm thick electron transporting layer. A concentration of the compound Liq in the electron transporting layer was 33 mass %.

Liq was deposited on the electron transporting layer to form a 1-nm thick electron injecting layer.

A metal Al was deposited on the electron injecting layer to form an 80-nm thick metal cathode.

Thus, an organic EL device of Example 3 was manufactured.

A device arrangement of the organic EL device of Example 3 is roughly shown as follows.

ITO(130)/HA(5)/HT16(105)/HT2(15)/H2:D2(20,4%)/ET:Liq(30,33%)/Liq(1)/Al(80)

Numerals in parentheses represent film thicknesses (unit: nm). The numerals represented by percentage in the same parentheses indicate a concentration (mass %) of the compound D2 in the emitting layer or a concentration (mass %) of Liq in the electron transporting layer.

### Example 4

An organic EL device of Example 4 was manufactured in the same manner as the organic EL device of Example 3 except that the compound HT17 was used in place of the compound HT16 in the first hole transporting layer of Example 3.

A device arrangement of the organic EL device of Example 4 is roughly shown as follows.

ITO(130)/HA(5)/HT17(105)/HT2(15)/H2:D2(20,4%)/ET:Liq(30,33%)/Liq(1)/Al(80)

### Example 5

An organic EL device of Example 5 was manufactured in the same manner as the organic EL device of Example 3 except that the compound HT18 was used in place of the compound HT16 in the first hole transporting layer of Example 3.

A device arrangement of the organic EL device of Example 5 is roughly shown as follows.

ITO(130)/HA(5)/HT18(105)/HT2(15)/H2:D2(20,4%)/ET:Liq(30,33%)/Liq(1)/Al(80)

### Comparative 5

An organic EL device of Comparative 5 was manufactured in the same manner as the organic EL device of Example 3 except that the compound HT11 was used in place of the compound HT16 in the first hole transporting layer of Example 3.

A device arrangement of the organic EL device of Comparative 5 is roughly shown as follows.

ITO(130)/HA(5)/HT11 (105)/HT2(15)/H2:D2(20,4%)/ET:Liq(30,33%)/Liq(1)/Al(80)

### Evaluation 2 of Organic EL Devices

The prepared organic EL devices of Examples 3 to 5 and Comparative 5 were also evaluated in the same manner as described above. The evaluation results are shown in Table 2.

TABLE 2

Compound

| | First Hole | | | |
|---|---|---|---|---|
| | Transporting | Emitting | Voltage | EQE |
| | Layer | Layer | [V] | [%] |
| Ex. 3 | HT16 | H2 | 3.28 | 8.4 |
| Ex. 4 | HT17 | H2 | 3.29 | 8.8 |
| Ex. 5 | HT18 | H2 | 3.16 | 8.5 |
| Comp. 5 | HT11 | H2 | 3.35 | 6.8 |

Comparison between the organic EL devices of Examples 3 to 5 and the organic EL device of Comparative 5, each of which used the compound D2 as the blue-emitting fluorescent compound, shows that the organic EL devices of Examples 3 to 5 each required a lower drive voltage and exhibited a higher external quantum efficiency (EQE) than the organic EL device of Comparative 5. The organic EL devices of Examples 3 to 5 also each included the emitting layer containing the first compound represented by the formula (1) and the blue-emitting fluorescent second compound, and the first hole transporting layer containing the third compound represented by the formula (3). Such an arrangement is supposed to improve the performance of the organic EL device.

### Manufacture 3 of Organic EL Device

### Example 6

An organic EL device of Example 6 was manufactured in the same manner as the organic EL device of Example 1 except that the compound HT19 was used in place of the compound HT14 in the first hole transporting layer of Example 1 and the compound H3 was used in place of the compound H2 in the emitting layer.

A device arrangement of the organic EL device of Example 6 is roughly shown as follows.

ITO(130)/HA(5)/HT19(80)/HT2(10)/H3:D1(25, 4%)/ET:Liq(25, 50%)/Liq(1)/Al(80)

### Example 7

An organic EL device of Comparative 7 was manufactured in the same manner as the organic EL device of Example 1 except that the compound H3 was used in place of the compound H2 in the emitting layer of Example 1.

A device arrangement of the organic EL device of Example 7 is roughly shown as follows.

ITO(130)/HA(5)/HT14(80)/HT2(10)/H3:D1(25, 4%)/ET:Liq(25, 50%)/Liq(1)/Al(80)

### Example 8

An organic EL device of Example 8 was manufactured in the same manner as the organic EL device of Example 1 except that the compound HT19 was used in place of the compound HT14 in the first hole transporting layer of Example 1 and the compound H4 was used in place of the compound H2 in the emitting layer.

A device arrangement of the organic EL device of Example 8 is roughly shown as follows.

ITO(130)/HA(5)/HT19(80)/HT2(10)/H4:D1(25, 4%)/ET:Liq(25, 50%)/Liq(1)/Al(80)

### Example 9

An organic EL device of Comparative 9 was manufactured in the same manner as the organic EL device of Example 1 except that the compound H4 was used in place of the compound H2 in the emitting layer of Example 1.

A device arrangement of the organic EL device of Example 9 is roughly shown as follows.

ITO(130)/HA(5)/HT14(80)/HT2(10)/H4:D1(25, 4%)/ET:Liq(25, 50%)/Liq(1)/Al(80)

### Evaluation 3 of Organic EL Devices

The prepared organic EL devices of Examples 6 to 9 were also evaluated in the same manner as described above. The evaluation results are shown in Table 3.

TABLE 3

Compound

| | First Hole | | | |
|---|---|---|---|---|
| | Transporting | Emitting | Voltage | EQE |
| | Layer | Layer | [V] | [%] |
| EX. 6 | HT19 | H3 | 3.33 | 9.3 |
| EX. 7 | HT14 | H3 | 3.20 | 9.0 |
| EX. 8 | HT19 | H4 | 3.41 | 9.4 |
| EX. 9 | HT14 | H4 | 3.24 | 9.4 |

The organic EL devices of Examples 6 to 9 also each included the emitting layer containing the first compound represented by the formula (1) and the blue-emitting fluorescent second compound, and the first hole transporting layer containing the third compound represented by the formula (3). The organic EL devices of Examples 6 to 9 thus achieved a drive voltage and an external quantum efficiency (EQE) substantially comparable to those of the organic EL device of Example 1.

As shown in Examples as described above, it has been demonstrated that a combination use of the first compound in the emitting layer and the third compound in the hole transporting layer according to the invention can provide an organic EL device exhibiting an improved external quantum efficiency (EQE) with a reduced drive voltage.

1. An organic electroluminescence device comprising:
an anode;
a cathode;
an emitting layer disposed between the anode and the cathode; and
a hole transporting layer disposed between the anode and the emitting layer, wherein
the emitting layer comprises a first compound represented by a formula (1) below and a fluorescent second compound, and
the hole transporting layer comprises a third compound represented by a formula (3) below, 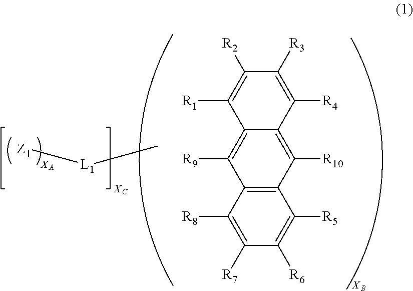
where: one of R₁ to R₁₀ is a single bond to L₁, and the rest of R₁ to R₁₀ not bonded to L₁ are each independently a hydrogen atom or a substituent; ones of R₁ to R₁₀ each in a form of the substituent are each independently selected from the group consisting of a halogen atom, a hydroxyl group, a cyano group, a substituted or unsubstituted amino group, a substituted or unsubstituted alkyl group having 1 to 30 carbon atoms, a substituted or unsubstituted alkoxy group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted aromatic hydrocarbon group having 6 to 30 ring carbon atoms, and a substituted or unsubstituted heterocyclic group having 5 to 30 ring atoms; L₁ is a single bond or a linking group; L₁ in a form of the linking group is a substituted or unsubstituted aromatic hydrocarbon group having 6 to 30 ring carbon atoms, or a substituted or unsubstituted heterocyclic group having 5 to 30 ring atoms; Z₁ is represented by a formula (1a) below; x_{A}, x_{B} and x_{C} are each independently an integer of 1 to 4; plural Z₁ are optionally the same or different; plural structures represented by [(Z₁)x_{A}-L₁-] are optionally the same or different; and plural cyclic structures in parentheses with a subscript x_{B} are optionally mutually the same or different, 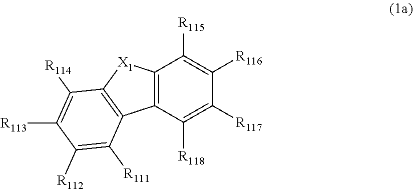
where: X₁ is an oxygen atom or a sulfur atom; R₁₁₁ to R₁₁₈ are each independently a hydrogen atom, a substituent or a single bond to L₁; R₁₁₁ to R₁₁₈ each in a form of the substituent are each independently selected from the group consisting of the substituents listed for R₁ to R₁₀ each in a form of the substituent; and when at least one of a pair of R₁₁₁ and R₁₁₂, a pair of R₁₁₂ and R₁₁₃, a pair of R₁₁₃ and R₁₁₄, a pair of R₁₁₅ and R₁₁₆, a pair of R₁₁₆ and R₁₁₇ or a pair of R₁₁₇ and R₁₁₈ are both substituents, the substituents are optionally bonded to each other to form a ring represented by a formula (1b) or (1c) below, 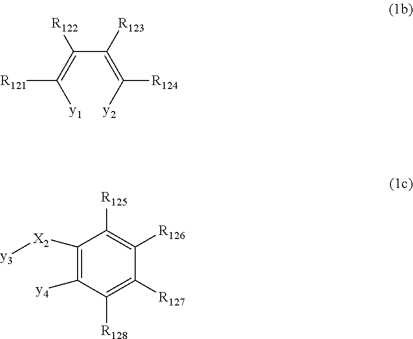
where: y₁ and y₂ in the formula (1b) each represent a position where a cyclic structure of Z₁ represented by the formula (1a) is bonded; y₃ and y₄ in the formula (1c) each represent a position where the cyclic structure of Z₁ represented by the formula (1a) is bonded; X₂ in the formula (1c) is an oxygen atom, a sulfur atom, NR_{D} or CR_{E}R_{F}; R₁₂₁ to R₁₂₄, R₁₂₅ to R₁₂₈, R_{D}, R_{E} and R_{F} in the formulae (1b) and (1c) are each independently a hydrogen atom, a substituent or a single bond to L₁; R₁₂₁ to R₁₂₄, R₁₂₅ to R₁₂₈, R_{D}, R_{E} and R_{F} each in a form of the substituent are each independently selected from the group consisting of the substituents listed for R₁ to R₁₀ each in a form of the substituent; and when a ring represented by the formula (1b) is formed, one of R₁₁₁ to R₁₁₈ and R₁₂₁ and R₁₂₄ not forming the ring is a single bond to L₁, and when a ring represented by the formula (1c) is formed, one of R₁₁₁ to R₁₁₈ and R₁₂₅ and R₁₂₈ not forming the ring is a single bond to L₁, 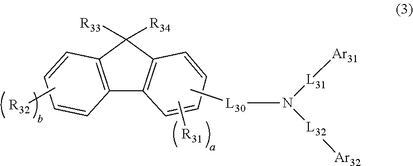
where: R₃₁ to R₃₂ are each independently a hydrogen atom or a substituent; R₃₁ to R₃₂ each in a form of the substituent are each independently selected from the group consisting of the substituents listed for R₁ to R₈ each in a form of the substituent; a is 3; plural R₃₁ are optionally mutually the same or different; R₃₁ each in a form of the substituent are optionally bonded together to form a cyclic structure; b is 4; plural R₃₂ are optionally mutually the same or different; R₃₂ each in a form of the substituent are optionally bonded together to form a cyclic structure; R₃₃ to R₃₄ are each independently a hydrogen atom or a substituent; R₃₃ to R₃₄ each in a form of the substituent are each independently selected from the group consisting of a substituted or unsubstituted alkyl group having 1 to 20 carbon atoms, a substituted or unsubstituted fluoroalkyl group having 1 to 20 carbon atoms, a substituted or unsubstituted aromatic hydrocarbon group having 6 to 50 ring carbon atoms, a substituted or unsubstituted heterocyclic group having 3 to 50 ring atoms, and a cyano group; R₃₃ and R₃₄ each in a form of the substituent are optionally bonded together to form a cyclic structure; L₃₀, L₃₁ and L₃₂ are each independently a single bond or a linking group; L₃₀, L₃₁ and L₃₂ each in a form of the linking group are each independently a substituted or unsubstituted aromatic hydrocarbon group having 6 to 30 ring carbon atoms or a substituted or unsubstituted heterocyclic group having 5 to 30 ring atoms; and Ar₃₁ and Ar₃₂ are each independently a substituted or unsubstituted aromatic hydrocarbon group having 6 to 30 ring carbon atoms or a substituted or unsubstituted heterocyclic group having 5 to 30 ring atoms.

2. The organic electroluminescence device according to claim 1, wherein X₁ is an oxygen atom.

3. The organic electroluminescence device according to claim 1, wherein each of X₁ and X₂ is an oxygen atom.

4. The organic electroluminescence device according to claim 1, wherein
at least one of the pair of R₁₁₁ and R₁₁₂, the pair of R₁₁₂ and R₁₁₃, the pair of R₁₁₃ and R₁₁₄, the pair of R₁₁₅ and R₁₁₆, the pair of R₁₁₆ and R₁₁₇ or the pair of R₁₁₇ and R₁₁₈ are both substituents, and
at least one of the pair of R₁₁₁ and R₁₁₂ both being the substituents, the pair of R₁₁₂ and R₁₁₃ both being the substituents, the pair of R₁₁₃ and R₁₁₄ both being the substituents, the pair of R₁₁₅ and R₁₁₆ both being the substituents, the pair of R₁₁₆ and R₁₁₇ both being the substituents or the pair of R₁₁₇ and R₁₁₈ both being the substituents are bonded to each other to form a ring represented by the formula (1b) or (1c).

5. The organic electroluminescence device according to claim 1, wherein Z₁ in the first compound is one selected from the group consisting of groups represented by formulae (11) to (13) below, 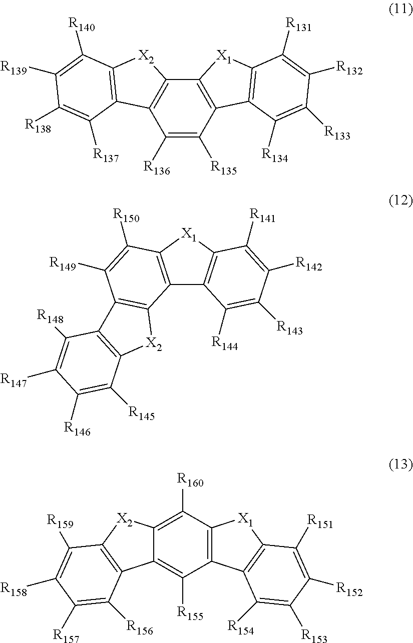
where: R₁₃₁ to R₁₄₀, R₁₄₁ to R₁₅₀ and R₁₅₁ to R₁₆₀ represent the same as the rest of R₁ to R₁₀ not bonded to L₁ in the formula (1); at least one of R₁₃₁ to R₁₄₀, at least one of R₁₄₁ to R₁₅₀ or at least one of R₁₅₁ to R₁₆₀ is a single bond to L₁; X₁ represents the same as X₁ in the formula (1a); X₂ represents the same as X₂ in the formula (1c); and X₁ and X₂ are mutually the same or different.

6. The organic electroluminescence device according to claim 1, wherein Z₁ in the first compound is one selected from the group consisting of groups represented by formulae (14) to (16) below, 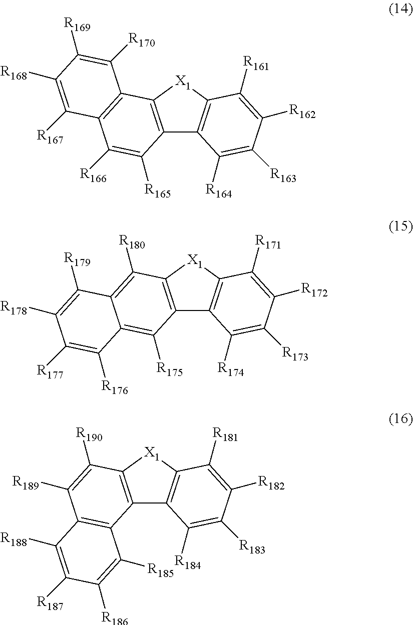
where: R₁₆₁ to R₁₇₀, R₁₇₁ to R₁₈₀ and R₁₈₁ to R₁₉₀ each independently represent the same as the rest of R₁ to R₁₀ not bonded to L₁ in the formula (1); and one of R₁₆₁ to R₁₇₀, one of R₁₇₁ to R₁₈₀ or one of R₁₈₁ to R₁₉₀ is a single bond to L₁; and X₁ represents the same as X₁ in the formula (1a).

7. The organic electroluminescence device according to claim 1, wherein x_{B} in the first compound is 1.

8. The organic electroluminescence device according to claim 1, wherein x_{A} in the first compound is 1 or 2.

9. The organic electroluminescence device according to claim 1, wherein L₁ in the first compound is bonded to R₉.

10. The organic electroluminescence device according to claim 1, wherein L₁ in the first compound is bonded to one of R₁ to R₄.

11. The organic electroluminescence device according to claim 10, wherein L₁ in the first compound is bonded to R₂.

12. The organic electroluminescence device according to claim 1, wherein the first compound is represented by a formula (10) below, 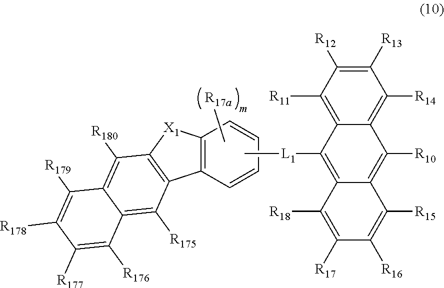
where: R₁₁ to R₁₈ are each independently a hydrogen atom or a substituent; R₁₁ to R₁₈ each in a form of the substituent are each independently selected from the group consisting of a halogen atom, a hydroxyl group, a cyano group, a substituted or unsubstituted amino group, a substituted or unsubstituted alkyl group having 1 to 30 carbon atoms, a substituted or unsubstituted alkoxy group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted aromatic hydrocarbon group having 6 to 30 ring carbon atoms, and a substituted or unsubstituted heterocyclic group having 5 to 30 ring atoms; L₁ is a single bond or a linking group; L₁ in a form of the linking group is a substituted or unsubstituted aromatic hydrocarbon group having 6 to 30 ring carbon atoms or a substituted or unsubstituted heterocyclic group having 5 to 30 ring atoms; R₁₀ is a substituted or unsubstituted aromatic hydrocarbon group having 6 to 30 ring carbon atoms or a substituted or unsubstituted heterocyclic group having 5 to 30 ring atoms; R₁₇ₐ is a hydrogen atom, a substituent, or a single bond to L₁; R₁₇ₐ in a form of the substituent is selected from the group consisting of the substituents listed for R₁₁ to R₁₈ each in a form of the substituent; m is 3; plural R₁₇ₐ are optionally mutually the same or different; X₁ represents the same as X₁ in the formula (1a); R₁₇₅ to R₁₈₀ are each independently a hydrogen atom or a substituent; and R₁₇₅ to R₁₈₀ each in a form of the substituent are each independently selected from the group consisting of the substituents listed for R₁₁ to R₁₈ each in a form of the substituent.

13. The organic electroluminescence device according to claim 12, wherein the first compound is represented by a formula (101) or a formula (102) below, 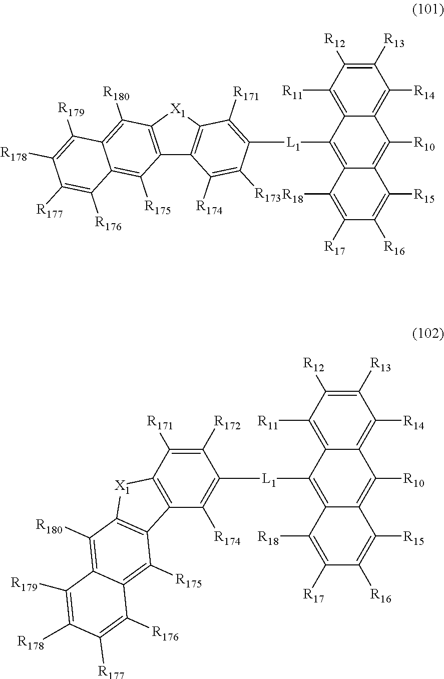
where: R₁₀, R₁₁ to R₁₈, L₁, X₁ and R₁₇₅ to R₁₈₀ respectively represent the same as R₁₀, R₁₀, R₁₁ to R₁₈, L₁, X₁ and R₁₇₅ to R₁₈₀ in the formula (10); R₁₇₁ to R₁₇₄ are each independently a hydrogen atom or a substituent; and R₁₇₁ to R₁₇₄ each in a form of the substituent are selected from the group consisting of the substituents listed for R₁₁ to R₁₈ each in a form of the substituent.

14. The organic electroluminescence device according to claim 13, wherein the first compound is represented by a formula (103) or a formula (104) below, 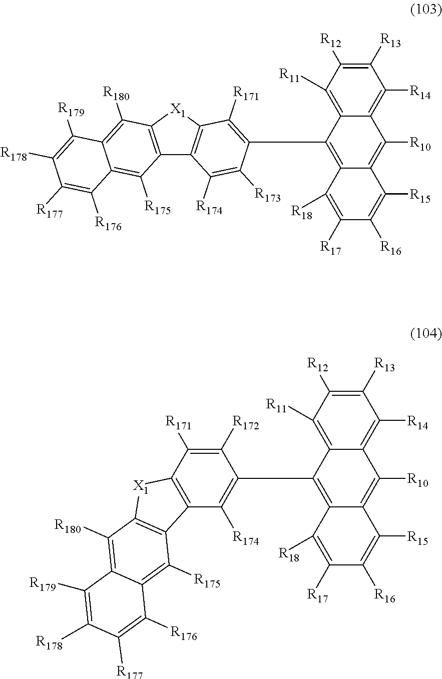
where: R₁₀, R₁₁ to R₁₈, X₁ and R₁₇₅ to R₁₈₀ respectively represent the same as R₁₀, R₁₁ to R₁₈, X₁ and R₁₇₅ to R₁₈₀ in the formula (10); R₁₇₁ to R₁₇₄ are each independently a hydrogen atom or a substituent; and R₁₇₁ to R₁₇₄ each in a form of the substituent are selected from the group consisting of the substituents listed for R₁₁ to R₁₈ each in a form of the substituent.

15. The organic electroluminescence device according to claim 1, wherein R₁₀ in the first compound is one selected from the group consisting of a substituted or unsubstituted aromatic hydrocarbon group having 6 to 30 ring carbon atoms and a substituted or unsubstituted heterocyclic group having 5 to 30 ring atoms.

16. The organic electroluminescence device according to claim 1, wherein R₁₀ in the first compound is a group represented by a formula (1d) below, 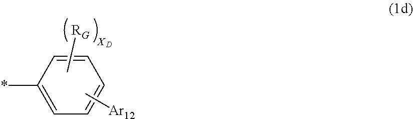
where: Ar₁₂ is one selected from the group consisting of a substituted or unsubstituted aromatic hydrocarbon group having 6 to 30 ring carbon atoms and a substituted or unsubstituted heterocyclic group having 5 to 30 ring atoms; R_{G} is a hydrogen atom or a substituent; R_{G} in a form of the substituent is selected from the group consisting of the substituents listed for R₁ to R₁₀ each in a form of the substituent; x_{D} is 4; and plural R_{G} are optionally mutually the same or different.

17. The organic electroluminescence device according to claim 15, wherein R₁₀ in the first compound is a substituted or unsubstituted fused aromatic hydrocarbon group having 10 to 30 ring carbon atoms.

18. The organic electroluminescence device according to claim 17, wherein R₁₀ in the first compound is a substituent selected from the group consisting of a substituted or unsubstituted naphthyl group, a substituted or unsubstituted phenanthryl group, a substituted or unsubstituted benzanthryl group, and a substituted or unsubstituted 9,9-dimethylfluorenyl group.

19. The organic electroluminescence device according to claim 1, wherein the third compound is represented by a formula (30) below, 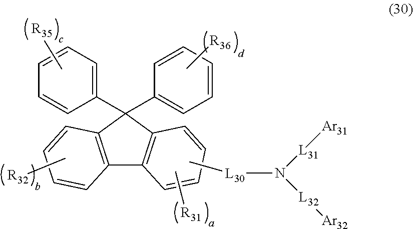
where: R₃₁, R₃₂, a, b, Ar₃₁, Ar₃₂, L₃₀, L₃₁ and L₃₂ respectively represent the same as R₃₁, R₃₂, a, b, Ar₃₁, Ar₃₂, L₃₀, L₃₁ and L₃₂ in the formula (3); R₃₅ and R₃₆ are each independently a hydrogen atom or a substituent; R₃₅ and R₃₆ each in a form of the substituent are each independently selected from the group consisting of the substituents listed for R₁ to R₈ each in a form of the substituent; c is 5; plural R₃₅ are optionally mutually the same or different; R₃₅ each in a form of the substituent are optionally bonded together to form a cyclic structure; d is 5; plural R₃₆ are optionally mutually the same or different; R₃₆ each in a form of the substituent are optionally bonded together to form a cyclic structure; and an aromatic ring bonded to R₃₅ is optionally single-bonded to an aromatic ring bonded to R₃₆.

20. The organic electroluminescence device according to claim 1, wherein the third compound is represented by a formula (31) below, 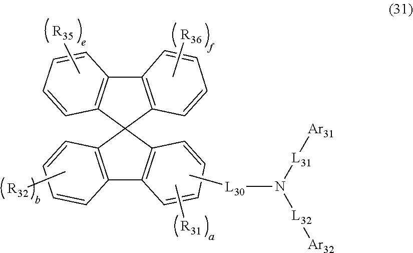
where: R₃₁ to R₃₂, a, b, L₃₀ to L₃₂, Ar₃₁ and Ar₃₂ each independently represent the same as R₃₁ to R₃₂, a, b, L₃₀ to L₃₂, Ar₃₁ and Ar₃₂ in the formula (3); R₃₅ and R₃₆ are each independently a hydrogen atom or a substituent; R₃₅ and R₃₆ each in a form of the substituent are each independently selected from the group consisting of the substituents listed for R₁ to R₈ each in a form of the substituent; each of e and f is 4; plural R₃₅ are optionally mutually the same or different; R₃₅ each in a form of the substituent are optionally bonded together to form a cyclic structure; plural R₃₆ are optionally mutually the same or different; and R₃₆ each in a form of the substituent are optionally bonded together to form a cyclic structure.

21. The organic electroluminescence device according to claim 19, wherein an aromatic ring bonded to R₃₅ is not bonded to an aromatic ring bonded to R₃₆ in the formula (30).

22. The organic electroluminescence device according to claim 1, wherein Ar₃₁ and Ar₃₂ are each independently one selected from the group consisting of groups represented by formulae (31a) to (31k) and (31m) below, 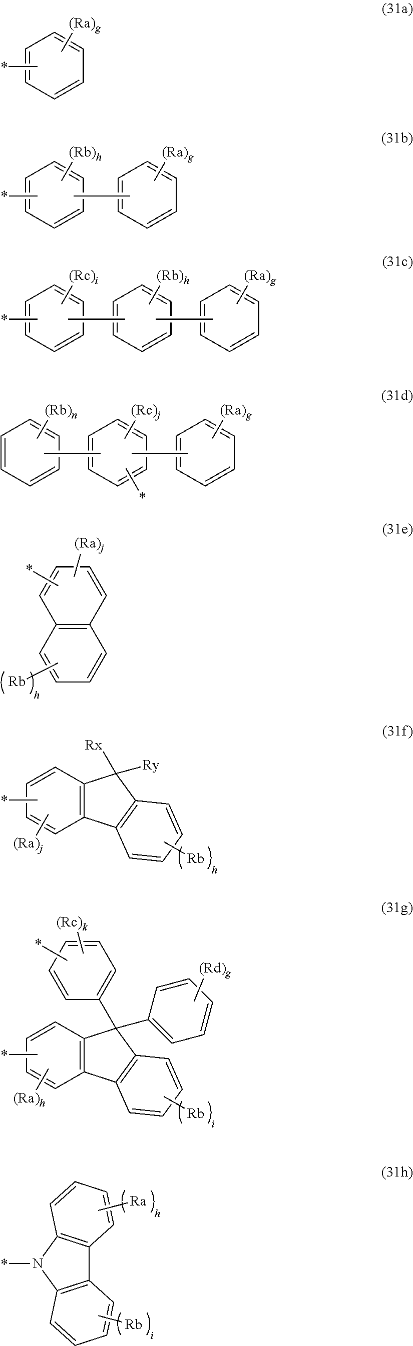 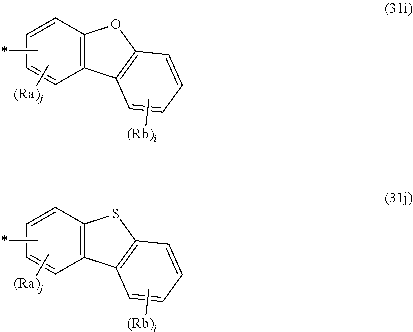
where: Ra, Rb, Re and Rd are each independently a hydrogen atom or a substituent; Ra, Rb, Re and Rd each in a form of the substituent are selected from the group consisting of the substituents listed for R₁ to R₈ each in a form of the substituent; Rx and Ry are each independently a hydrogen atom or a substituent; Rx and Ry each in a form of the substituent are each selected from the group consisting of a substituted or unsubstituted alkyl group having 1 to 30 carbon atoms and a substituted or unsubstituted aromatic hydrocarbon group having 6 to 30 ring carbon atoms; Rz is a hydrogen atom or a substituent; Rz in a form of the substituent is selected from the group consisting of a substituted or unsubstituted aromatic hydrocarbon group having 6 to 30 ring carbon atoms and a substituted or unsubstituted heterocyclic group having 5 to 30 ring atoms; g is 5, h is 4, i is 4, j is 3, k is 4, m is 4, and n is 5; plural Ra are optionally mutually the same or different; Ra each in a form of the substituent are optionally bonded together to form a cyclic structure; plural Rb are optionally mutually the same or different; Rb each in a form of the substituent are optionally bonded together to form a cyclic structure; plural Rc are optionally mutually the same or different; Rc each in a form of the substituent are optionally bonded together to form a cyclic structure; plural Rd are optionally mutually the same or different; Rd each in a form of the substituent are optionally bonded together to form a cyclic structure; and * represents a position where L₃₁ or L₃₂ is bonded.

23. The organic electroluminescence device according to claim 22, wherein Ar₃₁ and Ar₃₂ are each independently one selected from the group consisting of groups represented by formulae (31n) and (31p) to (31w) below, 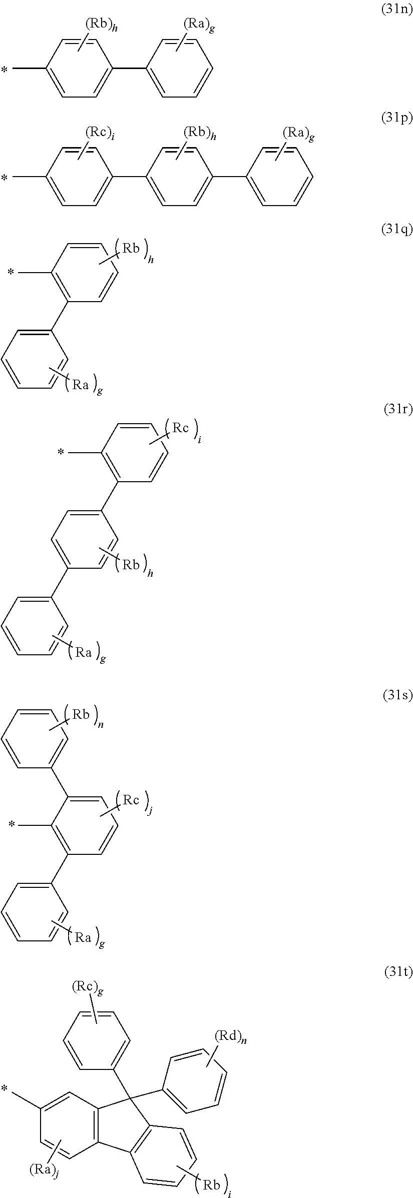 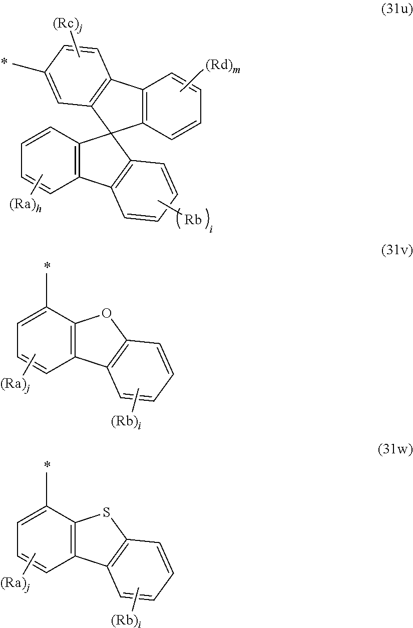
where: Ra, Rb, Rc, Rd, g, h, i, j, m, n and * respectively represent the same as Ra, Rb, Rc, Rd, g, h, i, j, m, n and * in the formulae (31a) to (31k) and (31m).

24. The organic electroluminescence device according to claim 1, wherein L₃₀, L₃₁ and L₃₂ are each independently a single bond or a linking group; and L₃₀, L₃₁ and L₃₂ each in a form of the linking group are each independently a substituted or unsubstituted aromatic hydrocarbon group having 6 to 30 ring carbon atoms.

25. The organic electroluminescence device according to claim 1, wherein L₃₀, L₃₁ and L₃₂ are each independently a linking group selected from the group consisting of linking groups represented by formulae (31x) to (31z) below, 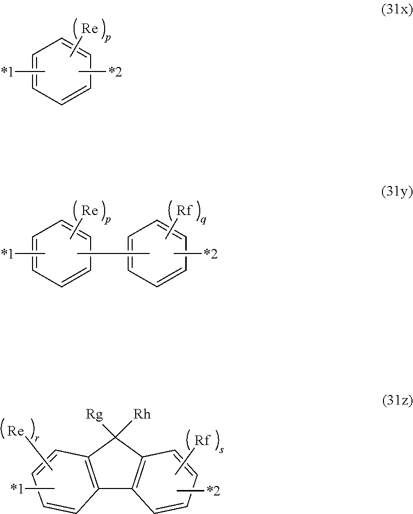
where: Re and Rf are each independently a hydrogen atom or a substituent; Re and Rf each in a form of the substituent are selected from the group consisting of the substituents listed for R₁ to R₈ each in a form of the substituent; Rg and Rh are each independently a hydrogen atom or a substituent; Rg and Rh each in a form of the substituent are each selected from the group consisting of a substituted or unsubstituted alkyl group having 1 to 30 carbon atoms and a substituted or unsubstituted aromatic hydrocarbon group having 6 to 30 ring carbon atoms; p is 4, q is 4, r is 3, and s is 3; plural Re are optionally mutually the same or different; Re each in a form of the substituent are optionally bonded together to form a cyclic structure; plural Rf are optionally mutually the same or different; Rf each in a form of the substituent are optionally bonded together to form a cyclic structure; and
*1 and *2 each represent a position for bonding to the nitrogen atom, Ar₃₁, Ar₃₂ or the fluorene ring in a structure represented by the formula (3).

26. The organic electroluminescence device according to claim 1, further comprising an electron transporting layer disposed between the cathode and the anode.

27. The organic electroluminescence device according to claim 1, further comprising a second hole transporting layer disposed between the hole transporting layer and the emitting layer.

28. An electronic device comprising the organic electroluminescence device according to claim 1.

29. The organic electroluminescence device according to claim 1, wherein
in the formula (1): R₁ to R₈ each represent a hydrogen atom; one of R₉ and R₁₀ represents a single bond for bonding to L₁; the other one of R₉ and R₁₀ represents a hydrogen atom or a substituent; R₉ and R₁₀ each in a form of the substituent are each independently selected from the group consisting of a substituted or unsubstituted aromatic hydrocarbon group having 6 to 30 ring carbon atoms, and a substituted or unsubstituted heterocyclic group having 5 to 30 ring atoms; L₁ is a single bond; x_{A} and x_{C} are each an integer of 1; and x_{B} is an integer of 1 to 4,
in the formula (1a): X₁ represents an oxygen atom; R₁₁₁ to R₁₁₈ each independently represent a hydrogen atom, a substituent or a single bond bonded to L₁; R₁₁₁ to R₁₁₈ each in a form of the substituent are each independently selected from the group consisting of a halogen atom, a hydroxyl group, a cyano group, a substituted or unsubstituted amino group, a substituted or unsubstituted alkyl group having 1 to 30 carbon atoms, a substituted or unsubstituted alkoxy group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted aromatic hydrocarbon group having 6 to 30 ring carbon atoms, and a substituted or unsubstituted heterocyclic group having 5 to 30 ring atoms; and when at least one of a pair of R₁₁₁ and R₁₁₂, a pair of R₁₁₂ and R₁₁₃, a pair of R₁₁₃ and R₁₁₄, a pair of R₁₁₅ and R₁₁₆, a pair of R₁₁₆ and R₁₁₇ or a pair of R₁₁₇ and R₁₁₈ are both substituents, the substituents are optionally bonded to each other to form a ring represented by the formula (1b),
in the formula (1b): R₁₂₁ to R₁₂₄ each independently represent a hydrogen atom, a substituent or a single bond bonded to L₁; and R₁₂₁ to R₁₂₄ each in a form of the substituent are each independently selected from the group consisting of a halogen atom, a hydroxyl group, a cyano group, a substituted or unsubstituted amino group, a substituted or unsubstituted alkyl group having 1 to 30 carbon atoms, a substituted or unsubstituted alkoxy group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted aromatic hydrocarbon group having 6 to 30 ring carbon atoms, and a substituted or unsubstituted heterocyclic group having 5 to 30 ring atoms, and
in the formula (3): R₃₁ to R₃₂ each represent a hydrogen atom; a is 3 and b is 4; R₃₃ to R₃₄ are each independently selected from the group consisting of a substituted or unsubstituted alkyl group having 1 to 20 carbon atoms, and a substituted or unsubstituted aromatic hydrocarbon group having 6 to 50 ring carbon atoms; R₃₃ and R₃₄ each in a form of the substituent are optionally bonded together to form a cyclic structure; L₃₀ in a form of the linking group is a substituted or unsubstituted aromatic hydrocarbon group having 6 to 30 ring carbon atoms or a substituted or unsubstituted heterocyclic group having 5 to 30 ring atoms; L₃₁ to L₃₂ each independently represent a single bond or a linking group selected from the group consisting of groups represented by formulae (31x) to (31z) below; and Ar₃₁ and Ar₃₂ are each independently one selected from the group consisting of group represented by formulae (31a) to (31k) and (31m) below, 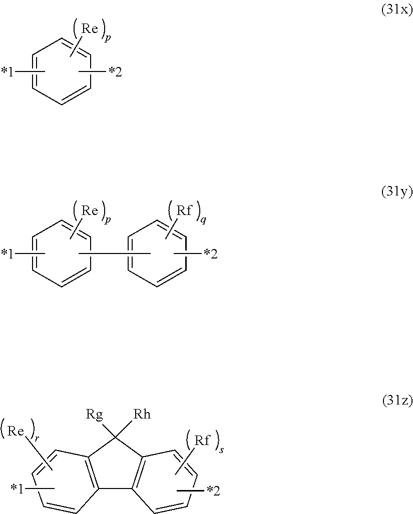
where: Re and Rf each independently represent a hydrogen atom or a substituent; Re and Rf each in a form of the substituent are each independently selected from the group consisting of a halogen atom, a hydroxyl group, a cyano group, a substituted or unsubstituted amino group, a substituted or unsubstituted alkyl group having 1 to 30 carbon atoms, a substituted or unsubstituted alkoxy group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted aromatic hydrocarbon group having 6 to 30 ring carbon atoms, and a substituted or unsubstituted heterocyclic group having 5 to 30 ring atoms; Rg and Rh are each independently a hydrogen atom or a substituent; Rg and Rh each in a form of the substituent are each selected from the group consisting of a substituted or unsubstituted alkyl group having 1 to 30 carbon atoms and a substituted or unsubstituted aromatic hydrocarbon group having 6 to 30 ring carbon atoms; p is 4, q is 4, r is 3, and s is 3; plural Re are optionally mutually the same or different; Re each in a form of the substituent are optionally bonded together to form a cyclic structure; plural Rf are optionally mutually the same or different; Rf each in a form of the substituent are optionally bonded together to form a cyclic structure; and *1 and *2 each represent a position for bonding to the nitrogen atom, Ar₃₁ or Ar₃₂ in the structure represented by the formula (3), 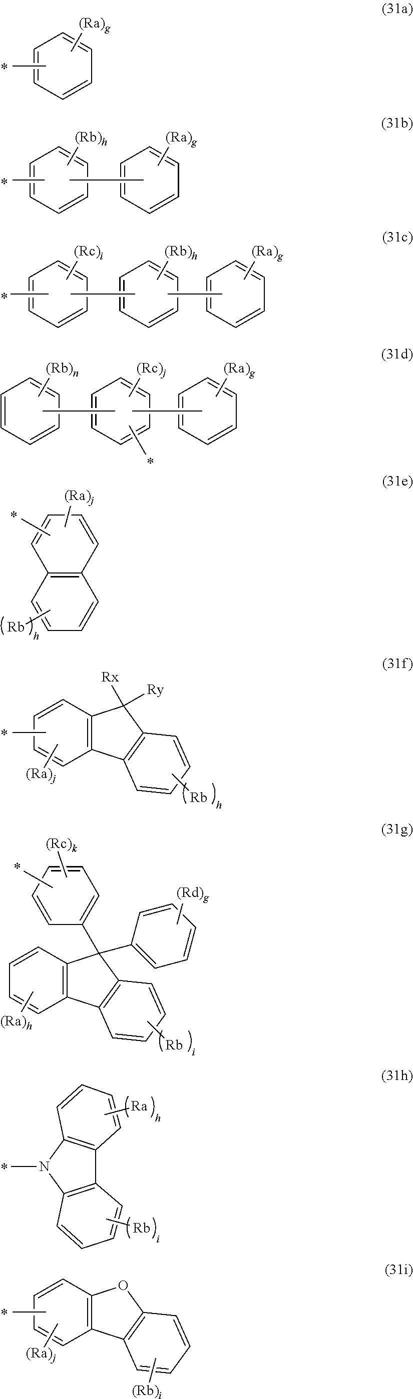 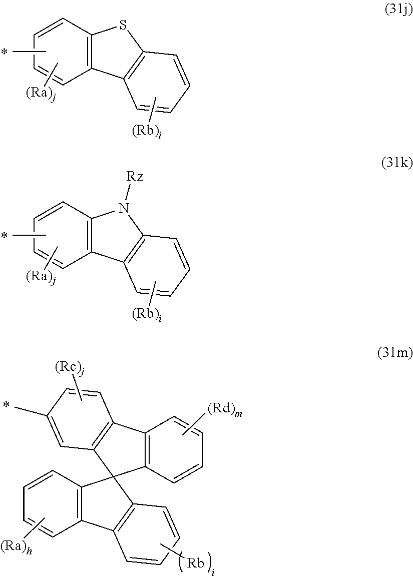
where: Ra, Rb, Rc and Rd each independently represent a hydrogen atom or a substituent; Ra, Rb, Rc and Rd each in a form of the substituent are each independently selected from the group consisting of a halogen atom, a hydroxyl group, a cyano group, a substituted or unsubstituted amino group, a substituted or unsubstituted alkyl group having 1 to 30 carbon atoms, a substituted or unsubstituted alkoxy group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted aromatic hydrocarbon group having 6 to 30 ring carbon atoms, and a substituted or unsubstituted heterocyclic group having 5 to 30 ring atoms; Rx and Ry each independently represent a hydrogen atom or a substituent; Rx and Ry each in a form of the substituent are each selected from the group consisting of a substituted or unsubstituted alkyl group having 1 to 30 carbon atoms and a substituted or unsubstituted aromatic hydrocarbon group having 6 to 30 ring carbon atoms; Rz represents a hydrogen atom or a substituent; Rz in a form of the substituent is selected from the group consisting of a substituted or unsubstituted aromatic hydrocarbon group having 6 to 30 ring carbon atoms and a substituted or unsubstituted heterocyclic group having 5 to 30 ring atoms; g is 5, h is 4, i is 4, j is 3, k is 4, m is 4, and n is 5; plural Ra are optionally mutually the same or different; Ra each in a form of the substituent are optionally bonded together to form a cyclic structure; plural Rb are optionally mutually the same or different; Rb each in a form of the substituent are optionally bonded together to form a cyclic structure; plural Rc are optionally mutually the same or different; Rc each in a form of the substituent are optionally bonded together to form a cyclic structure; plural Rd are optionally mutually the same or different; Rd each in a form of the substituent are optionally bonded together to form a cyclic structure; and * represents a position where L₃₁ or L₃₂ is bonded.
